(19) United States
(12) Patent Application Publication
(10) Pub. No.: US 2023/0203043 A1
(43) Pub. Date: Jun. 29, 2023

(54) NOVEL IMIDAZOPYRAZINE DERIVATIVES

(71) Applicant: Hoffmann-La Roche Inc., Little Falls, NJ (US)

(72) Inventors: Jean-Baptiste BLANC, Uffheim (FR); Christian LERNER, Bottmingen (CH); Matthias NETTEKOVEN, Grenzach-Wyhlen (DE); Philippe PFLIEGER, Schwoben (FR); Bernd PUELLMANN, Aesch (CH); Sébastien SCHMITT, Hagenthal-le-Bas (FR); Theodor STOLL, Binningen (CH); Song YANG, Shanghai (CN); Chengang ZHOU, Shanghai (CN)

(73) Assignee: Hoffmann-La Roche Inc., Little Falls, NJ (US)

(21) Appl. No.: 17/976,651

(22) Filed: Oct. 28, 2022

Related U.S. Application Data
(63) Continuation of application No. PCT/EP2021/060884, filed on Apr. 27, 2021.

(30) Foreign Application Priority Data
Apr. 29, 2020 (EP) 20171965.5
Mar. 19, 2021 (WO) PCT/CN2021/081790

(51) Int. Cl. C07D 487/04; C07D 519/00

(52) U.S. Cl. CPC C07D 487/04; C07D 519/00

(57) ABSTRACT

The invention provides novel imidazopyrazine derivatives having the general formula (I), or pharmaceutically acceptable salts thereof, wherein X and R³ to R⁹ are as described herein:

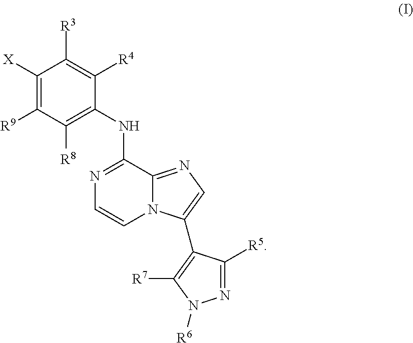

Further provided are pharmaceutical compositions including the compounds, processes of manufacturing the compounds and methods of using the compounds as medicaments, in particular methods of using the compounds as antibiotics for the treatment or prevention of bacterial infections and resulting diseases.

### CROSS-REFERENCE TO RELATED APPLICATIONS

This application is a Continuation of International Patent Application No. PCT/EP2021/060884, filed Apr. 27, 2021, which claims benefit of priority to International Patent Application No. PCT/CN2021/081790, filed Mar. 19, 2021 and European Application No. 20171965.5, filed Apr. 29, 2020, each of which is incorporated herein by reference in its entirety.

### BACKGROUND

The present invention relates to novel imidazopyrazine derivatives which exhibit antibacterial properties. The invention also relates to methods of using the compounds for the treatment or prevention of bacterial infections and resulting diseases, in particular for the treatment or prevention of infections with Acinetobacter baumannii and resulting diseases.

Acinetobacter baumannii is a Gram-negative, aerobic, nonfermenting bacterium recognized over the last decades as an emergining pathogen with very limited treatment options. A. baumannii is considered to be a serious threat by the US Centers for Disease Control and Prevention and belongs to the so called ‘ESKAPE’ pathogens (Enterococcus faecium, Staphylococcus aureus, Klebsiella pneumoniae, Acinetobacter baumannii, Pseudomonas aeruginosa and Enterobacter species & E. coli) that currently cause the majority of nosocomial infections and effectively “escape” the activity of antimicrobial agents. A. baumannii is most often encountered in intensive care units and surgical wards, where extensive antibiotic use has enabled selection for resistance against all known antimicrobials and where it causes infections that include bacteremia, pneumonia, meningitis, urinary tract infection, and wound infection.

A. baumannii has an exceptional ability to upregulate and acquire resistance determinants and shows an environmental persistance that allows its survival and spread in the nosocomial setting, making this organism a frequent cause of outbreaks of infection and an endemic, health care-associated pathogen.

Due to increasing antibiotic resistance to most if not all available therapeutic options, Muti-Drug Resistant (MDR) A. baumannii infections, especially those caused by Carbapenem resistant A. baumannii, are extremely difficult or even impossible to treat with high mortality rate as well as increased morbidity and length of stay in intensive care unit.

Acinetobacter baumannii has been defined and still remains “a prime example of a mismatch between unmet medical needs and the current antimicrobial research and development pipeline” according to the Antimicrobial Availability Task Force (AATF) of the Infectious Diseases Society of America (IDSA). Thus, there is a high demand and need to identify compounds suitable for the treatment of diseases and infections caused by Acinetobacter baumannii.

The present invention provides novel compounds which exhibit activity against drug-susceptible as well as drug-resistant strains of Acinetobacter baumannii.

### SUMMARY OF THE DISCLOSURE

In a first aspect, the present invention provides compounds of formula (I)

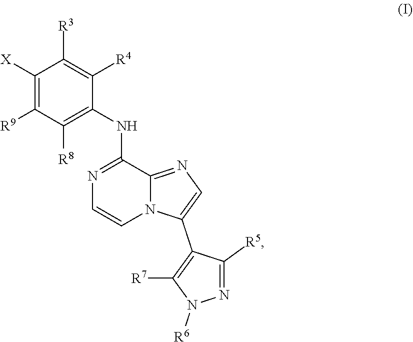

or pharmaceutically acceptable salts thereof, wherein X and R³ to R⁹ are as defined herein.

In one aspect, the present invention provides a process of manufacturing the compounds of formula (I) described herein, comprising:
(i) reacting a carboxylic acid IXa, wherein R³ to R⁹ are as defined herein,

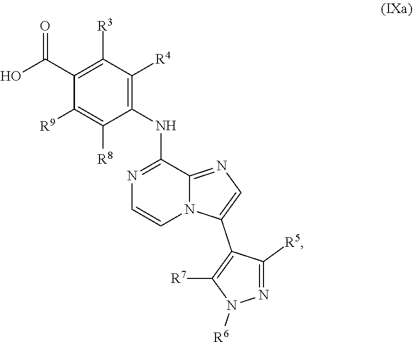

with an amine V, wherein R¹ and R² are as defined herein,

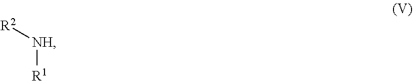

in the presence of a coupling reagent (such as HATU, TBTU, and the like) and a base (such as DIPEA, NEt₃, and the like), optionally in a solvent (such as DMF, dioxane, THF, and the like) to form said compound of formula (I); or (ii) reacting a compound VIa, wherein R¹ to R⁴, R⁸, and R⁹ are as defined herein and Z is a halogen or a triflate,

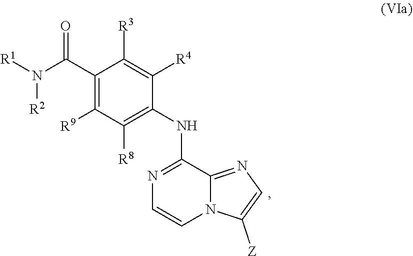

with a compound VIIa, wherein R⁵ to R⁷ are as defined herein and Y is a boronic acid or a boronic acid ester,

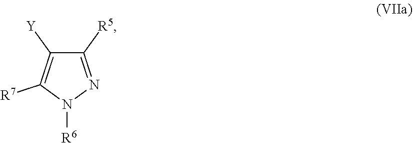

in the presence of a transition metal catalyst (such as PdCl₂(dppf)-CH₂Cl₂ adduct, Pd(PPh₃)₄, and the like) and a base (such as NEt₃, DIPEA, carbonates, K₃PO₄, NaOtBu, and the like) in a solvent (such as dioxane, DMF, THF and the like) to form said compound of formula (I).

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a further aspect, the present invention provides a compound of formula (I) as described herein, or a pharmaceutically acceptable salt thereof, for use in the treatment or prevention of infections and resulting diseases caused by Gram-negative bacteria.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

### DETAILED DESCRIPTION OF THE DISCLOSURE

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The following definitions are provided to facilitate understanding of certain terms used frequently herein and are not meant to limit the scope of the present disclosure. All references referred to herein are incorporated by reference in their entirety.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. A particularly preferred, yet non-limiting example of alkyl is methyl.

The term “alkenyl” denotes a monovalent linear or branched hydrocarbon group of 2 to 6 carbon atoms with at least one double bond (“C₂-C₆-alkenyl”). In particular embodiments, alkenyl has 2 to 4 carbon atoms with at least one double bond. Examples of alkenyl include ethenyl, propenyl, prop-2-enyl, isopropenyl, n-butenyl and iso-butenyl. Particular alkenyl group is ethenyl.

The term “alkynyl” denotes a monovalent linear or branched hydrocarbon group of 2 to 6 carbon atoms with at least one triple bond (“C₂-C₆-alkynyl”). In particular embodiments, alkynyl has 2 to 4 carbon atoms with at least one triple bond. Examples of alkynyl include ethynyl, propynyl, n-butynyl or isobutynyl. Preferred alkenyl is propynyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred, yet non-limiting examples of “halogen” or “halo” are fluoro (F) and chloro (Cl).

The term “cycloalkyl” as used herein refers to a saturated or partly unsaturated monocyclic or bicyclic hydrocarbon group of 3 to 12 ring carbon atoms (“C₃-C₁₂-cycloalkyl”). In some preferred embodiments, the cycloalkyl group is a saturated monocyclic hydrocarbon group of 3 to 10 ring carbon atoms, in particular 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl and cycloheptyl.

The term “aminoalkoxy” refers to an alkoxy group, wherein at least one of the hydrogen atoms of the alkoxy group has been replaced by an amino group. Preferably, “aminoalkoxy” refers to an alkoxy group wherein 1, 2 or 3 hydrogen atoms of the alkoxy group have been replaced by an amino group. A preferred, yet non-limiting example of aminoalkoxy is aminomethoxy.

The term “aminoalkyl” refers to an alkyl group, wherein at least one of the hydrogen atoms of the alkyl group has been replaced by an amino group. Preferably, “aminoalkyl” refers to an alkyl group wherein 1, 2 or 3 hydrogen atoms of the alkyl group have been replaced by an amino group. A preferred, yet non-limiting example of aminoalkyl is aminomethyl.

The terms “heterocycloalkyl” and “heterocyclyl” are used interchangeably and refer to a saturated or partly unsaturated mono- or bicyclic, preferably monocyclic ring system of 3 to 20 ring atoms, preferably 3 to 15 ring atoms, more preferably 3 to 10 ring atoms, most preferably 3 to 6 ring atoms, wherein 1, 2, or 3 of said ring atoms are heteroatoms selected from N, O and S, the remaining ring atoms being carbon (“C₁-C₁₉-heterocyclyl”). Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of monocyclic heterocyclyl groups include azetidin-3-yl, azetidin-2-yl, oxetan-3-yl, oxetan-2-yl, 2-oxopyrrolidin-1-yl, 2-oxopyrrolidin-3-yl, 5-oxopyrrolidin-2-yl, 5-oxopyrrolidin-3-yl, 2-oxo-1-piperidyl, 2-oxo-3-piperidyl, 2-oxo-4-piperidyl, 6-oxo-2-piperidyl, 6-oxo-3-piperidyl, 1-piperidinyl, 2-piperidinyl, 3-piperidinyl, 4-piperidinyl, morpholino, morpholin-2-yl and morpholin-3-yl.

The term “aryl” refers to a monocyclic, bicyclic, or tricyclic carbocyclic ring system having a total of 6 to 14 ring members (“C₆-C₁₄-aryl”), preferably, 6 to 12 ring members, and more preferably 6 to 10 ring members, and wherein at least one ring in the system is aromatic. Some non-limiting examples of aryl include phenyl and 9H-fluorenyl (e.g. 9H-fluoren-9-yl). A particularly preferred, yet non-limiting example of aryl is phenyl.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, imidazol-1-yl, 1H-imidazol-2-yl, 1H-imidazol-4-yl, 1H-imidazol-5-yl, oxazol-2-yl, oxazol-4-yl, oxazol-5-yl, thiazolyl, pyrazolyl, pyridazinyl, pyrimidinyl, isoxazolyl, and oxadiazolyl.

The term “hydroxy” refers to an —OH group.

The term “amino” refers to an —NH₂ group.

The term “cyano” refers to a —CN (nitrile) group.

The term “carboxy” refers to a —COOH group.

The term “guanidine” refers to a

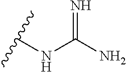 [SMILES: *NC(=N)N]

group.

The term “carbamoyl” refers to a —C(O)NH₂ group.

The term “carbonyl” refers to a —C(O)— group.

The term “alkoxycarbonyl” refers to a —C(O)—O-alkyl group (i.e., an alkyl ester).

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Particularly preferred, yet non-limiting examples of haloalkyl are trifluoromethyl and trifluoroethyl.

The term “haloalkenyl” refers to an alkenyl group, wherein at least one of the hydrogen atoms of the alkenyl group has been replaced by a halogen atom, preferably fluoro. Preferably, “haloalkenyl” refers to an alkenyl group wherein 1, 2 or 3 hydrogen atoms of the alkenyl group have been replaced by a halogen atom, most preferably fluoro. Particularly preferred, yet non-limiting examples of haloalkenyl are 2-chloroallyl and 2-chloro-1-methyl-allyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. A particularly preferred, yet non-limiting example of haloalkoxy is trifluoromethoxy (—OCF₃).

The term “cyanoalkyl” refers to an alkyl group, wherein at least one of the hydrogen atoms of the alkyl group has been replaced by a cyano group. Preferably, “cyanoalkyl” refers to an alkyl group wherein 1, 2 or 3 hydrogen atoms of the alkyl group have been replaced by a cyano group.

A particularly preferred, yet non-limiting example of cyanoalkyl is cyanomethyl. The term “cycloalkylalkyl” refers to an alkyl group, wherein at least one of the hydrogen atoms of the alkyl group has been replaced by a cycloalkyl group. Preferably, “cycloalkylalkyl” refers to an alkyl group wherein 1, 2 or 3 hydrogen atoms, most preferably 1 hydrogen atom of the alkyl group have been replaced by a cycloalkyl group. Particularly preferred, yet non-limiting examples of cycloalkylalkyl are cyclopropylmethyl and cyclobutylmethyl.

The term “alkyldiyl” as used herein refers to a saturated linear or branched-chain divalent hydrocarbon radical of about one to six carbon atoms (“C₁-C₆”). Examples of alkyldiyl groups include, but are not limited to, methylene (—CH₂—), ethylene (—CH₂CH₂—), propylene (—CH₂CH₂CH₂—), and the like. An alkyldiyl group may also be referred to as an “alkylene” group.

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl and hydroxyethyl (e.g. 2-hydroxyethyl). A particularly preferred, yet non-limiting example of hydroxyalkyl is hydroxymethyl.

The term “hydroxyheterocyclyl” refers to a heterocyclyl group, wherein at least one of the hydrogen atoms of the heterocyclyl group has been replaced by a hydroxy group. Preferably, “hydroxyheterocyclyl” refers to a heterocyclyl group wherein 1, 2 or 3 hydrogen atoms, most preferably 1 hydrogen atom of the heterocyclyl group have been replaced by a hydroxy group. A particularly preferred, yet non-limiting example of hydroxyheterocyclyl is 4-hydroxypyrrolidin-2-yl.

The term “arylalkyl” refers to an alkyl group, wherein at least one of the hydrogen atoms of the alkyl group has been replaced by an aryl group. Preferably, “arylalkyl” refers to an alkyl group wherein 1, 2 or 3 hydrogen atoms, most preferably 1 hydrogen atom of the alkyl group have been replaced by an aryl group. Particularly preferred, yet non-limiting examples of arylalkyl are benzyl, phenylethyl (in particular 2-phenylethyl), and phenylpropyl (in particular 3-phenylpropyl).

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as formic acid, acetic acid, trifluoroacetic acid, propionic acid, glycolic acid, pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, formates, fumarates, lactates (in particular derived from L-(+)-lactic acid), tartrates (in particular derived from L-(+)-tartaric acid) and trifluoroacetates.

The term “protective group” (PG) denotes the group which selectively blocks a reactive site in a multifunctional compound such that a chemical reaction can be carried out selectively at another unprotected reactive site in the meaning conventionally associated with it in synthetic chemistry. Protective groups can be removed at the appropriate point. Exemplary protective groups are amino-protective groups, carboxy-protective groups or hydroxy-protective groups. Particular protective groups are the tert-butoxycarbonyl (Boc), benzyloxycarbonyl (Cbz), fluorenylmethoxycarbonyl (Fmoc) and benzyl (Bn). Further particular protective groups are the tert-butoxycarbonyl (Boc) and the fluorenylmethoxycarbonyl (Fmoc). More particular protective group is the tert-butoxycarbonyl (Boc). Exemplary protective groups and their application in organic synthesis are described, for example, in “Protective Groups in Organic Chemistry” by T. W. Greene and P. G. M. Wutts, 5th Ed., 2014, John Wiley & Sons, N.Y.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prevention” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “mammal” as used herein includes both humans and non-humans and includes but is not limited to humans, non-human primates, canines, felines, murines, bovines, equines, and porcines. In a particularly preferred embodiment, the term “mammal” refers to humans.

The term “nosocomial infection” refers to a hospital-acquired infection (HAI), which is an infection that is acquired in a hospital or other health care facility. To emphasize both hospital and nonhospital settings, it is sometimes instead called a health care-associated infection (HAI or HCAI). Such an infection can be acquired in hospitals, nursing homes, rehabilitation facilities, outpatient clinics, or other clinical settings.

Compounds

In a first aspect, the present invention provides compounds of formula (I)

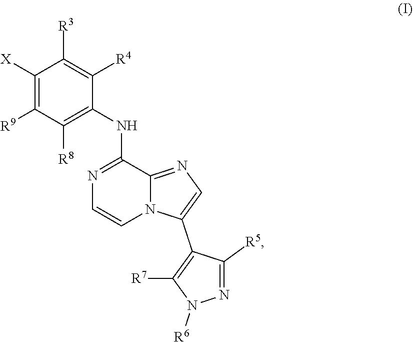

or pharmaceutically acceptable salts thereof, wherein:
(a) X is

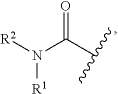

wherein:
   (i) R¹ and R², taken together with the N-atom to which they are attached, form an optionally substituted C₁-C₁₉-heterocycle; or
   (ii) R¹ is selected from the group consisting of:
      (i) hydrogen;
      (ii) optionally substituted C₁-C₆-alkyl;
      (iii) optionally substituted C₂-C₆-alkenyl;
      (iv) optionally substituted C₂-C₆-alkynyl;
      (v) optionally substituted C₁-C₆-alkoxy;
      (vi) optionally substituted C₁-C₁₉-heterocyclyl; and
      (vii) optionally substituted C₃-C₁₂-cycloalkyl; and
      R² is hydrogen or C₁-C₆-alkyl; or
(b) X is

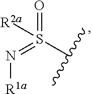

wherein:
   R^{1a} is selected from the group consisting of:
   (i) optionally substituted C₁-C₆-alkyl;
   (ii) optionally substituted C₂-C₆-alkenyl;
   (iii) optionally substituted C₂-C₆-alkynyl;
   (iv) optionally substituted C₁-C₁₉-heterocyclyl; and
   (v) optionally substituted C₃-C₁₂-cycloalkyl; and
   R² is C₁-C₆-alkyl;
R³, R⁴, R⁸, and R⁹ are independently selected from the group consisting of hydrogen, halogen, and C₁-C₆-alkyl;
R⁵ is selected from the group consisting of halogen, cyano, C₂-C₆-alkenyl, C₂-C₆-alkynyl, halo-C₂-C₆-alkenyl, optionally substituted C₁-C₆-alkyl, C₁-C₆-alkyl-S—, and a group

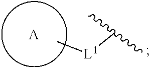

R⁶ and R⁷ are independently selected from the group consisting of hydrogen, halogen, cyano, C₂-C₆-alkenyl, C₂-C₆-alkynyl, halo-C₂-C₆-alkenyl, optionally substituted C₁-C₆-alkyl, C₁-C₆-alkyl-S—, and a group

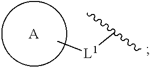

L¹ is selected from the group consisting of carbonyl, a covalent bond and C₁-C₆-alkyldiyl; and
A is selected from the group consisting of optionally substituted C₃-C₁₂-cycloalkyl, optionally substituted C₁-C₁₉-heterocyclyl, optionally substituted C₆-C₁₄-aryl, and optionally substituted C₁-C₁₃-heteroaryl.

In one embodiment, the present invention provides a compound of formula (I)

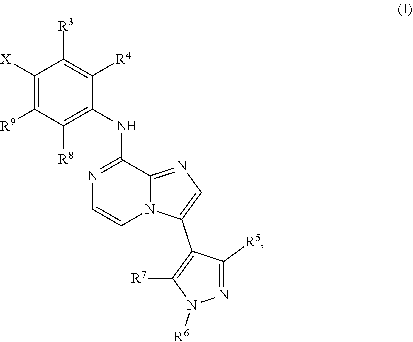

or pharmaceutically acceptable salts thereof, wherein:
(c) X is

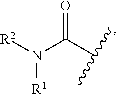

wherein:
   (iii) R¹ and R², taken together with the N-atom to which they are attached, form an optionally substituted C₁-C₁₉-heterocycle; or
   (iv) R¹ is selected from the group consisting of:
      (viii) hydrogen;
      (ix) optionally substituted C₁-C₆-alkyl;
      (x) optionally substituted C₂-C₆-alkenyl;
      (xi) optionally substituted C₂-C₆-alkynyl;
      (xii) optionally substituted C₁-C₆-alkoxy;
      (xiii) optionally substituted C₁-C₁₉-heterocyclyl; and
      (xiv) optionally substituted C₃-C₁₂-cycloalkyl; and
      R² is hydrogen or C₁-C₆-alkyl; or
(d) X is

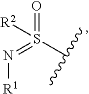

wherein:
   R¹ is selected from the group consisting of:
   (vi) optionally substituted C₁-C₆-alkyl;
   (vii) optionally substituted C₂-C₆-alkenyl;
   (viii) optionally substituted C₂-C₆-alkynyl;
   (ix) optionally substituted C₁-C₁₉-heterocyclyl; and
   (x) optionally substituted C₃-C₁₂-cycloalkyl; and
   R² is C₁-C₆-alkyl;
R³, R⁴, R⁸, and R⁹ are independently selected from the group consisting of hydrogen, halogen, and C₁-C₆-alkyl;
R⁵, R⁶, and R⁷ are independently selected from the group consisting of hydrogen, C₂-C₆-alkenyl, C₂-C₆-alkynyl, halo-C₂-C₆-alkenyl, optionally substituted C₁-C₆-alkyl, and a group

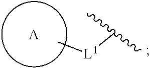

L¹ is selected from the group consisting of carbonyl, a covalent bond and C₁-C₆-alkyldiyl; and
A is selected from the group consisting of optionally substituted C₃-C₁₂-cycloalkyl, optionally substituted C₁-C₁₉-heterocyclyl, optionally substituted C₆-C₁₄-aryl, and optionally substituted C₁-C₁₃-heteroaryl.

In one embodiment, the present invention provides a compound of formula (I) as described herein, or a pharmaceutically acceptable salt thereof, wherein:
(a) X is

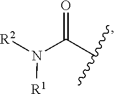

wherein:
   (i) R¹ and R², taken together with the N-atom to which they are attached, form a C₁-C₁₉-heterocycle, wherein the C₁-C₁₉-heterocycle is optionally substituted with one or more, preferably 1, 2, 3 or 4 substituents selected from the group consisting of R¹⁷—C₁-C₆-alkyl-L⁴-, amino, hydroxy, and a group

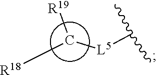

(ii) R¹ is selected from the group consisting of:
   (i) hydrogen;
   (ii) C₁-C₆-alkyl optionally substituted with one or more, preferably 1, 2, 3 or 4 substituents selected from the group consisting of C₂-C₆-alkenyl-NH—, C₁-C₆-alkoxy, C₁-C₆-alkoxycarbonyl-NH—, C₂-C₆-alkynyl-NH—, amino, halogen, cyano, hydroxy, and a group

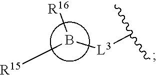

(iii) R¹⁰—C₁-C₆-alkyl-L²-C₁-C₆-alkyl-;
(iv) C₂-C₆-alkynyl;
(v) amino-C₁-C₆-alkoxy; and
(vi) a group

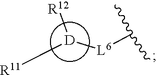

and
R² is hydrogen or C₁-C₆-alkyl; or
(b) X is

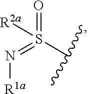

wherein:
   R^{1a} is selected from the group consisting of:
   (i) C₁-C₆-alkyl optionally substituted with one or more, preferably 1, 2, 3 or 4 substituents selected from the group consisting of C₂-C₆-alkenyl-NH—, C₁-C₆-alkoxy, C₁-C₆-alkoxycarbonyl-NH—, C₂-C₆-alkynyl-NH—, amino, halogen, cyano, hydroxy, and a group

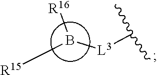

(ii) R¹⁰—C₁-C₆-alkyl-L²-C₁-C₆-alkyl-;
(iii) C₂-C₆-alkynyl; and
(iv) a group

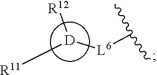

and
R² is C₁-C₆-alkyl;
R³, R⁴, R⁸, and R⁹ are independently selected from the group consisting of hydrogen, halogen, and C₁-C₆-alkyl;
R⁵ is selected from the group consisting of halogen, cyano, C₁-C₆-alkyl, C₂-C₆-alkenyl, C₂-C₆-alkynyl, halo-C₂-C₆-alkenyl, CF₃, C₁-C₆-alkyl-S—, and a group

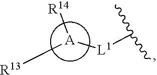

wherein said C₁-C₆-alkyl is optionally substituted with one or more, preferably 1, 2, 3 or 4 substituents independently selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R⁶ and R⁷ are independently selected from the group consisting of hydrogen, halogen, cyano, C₁-C₆-alkyl, C₂-C₆-alkenyl, C₂-C₆-alkynyl, halo-C₂-C₆-alkenyl, CF₃, C₁-C₆-alkyl-S—, and a group

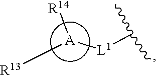

wherein said C₁-C₆-alkyl is optionally substituted with one or more, preferably 1, 2, 3 or 4 substituents independently selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R¹⁰ is selected from the group consisting of amino, C₁-C₆-alkyl-NH—, (C₁-C₆-alkyl)₂N—, C₂-C₆-alkynyl, amino-C₁-C₆-alkyl-NH—, carbamoyl, guanidine, and C₁-C₁₉-heterocyclyl, wherein the C₁-C₁₉-heterocyclyl is optionally substituted with one or two substituents selected from hydroxy and cyano-C₁-C₆-alkyl;
R¹¹ and R¹² are independently selected from the group consisting of amino, hydroxy, hydroxy-C₁-C₁₉-heterocyclyl-C(O)—, and hydroxy-C₁-C₁₉-heterocyclyl-C(O)—NH—;
R¹³ and R¹⁴ are independently selected from the group consisting of hydrogen, halogen, cyano, C₁-C₆-alkyl, amino, hydroxy, amino-C₁-C₆-alkyl, cyano-C₁-C₆-alkyl, hydroxy-C₁-C₆-alkyl, halo-C₁-C₆-alkyl, C₁-C₆-alkoxy, C₆-C₁₄-aryl-C₁-C₆-alkyl-, and C₃-C₁₂-cycloalkyl-C₁-C₆-alkyl-;
R¹⁵ and R¹⁶ are independently selected from the group consisting of hydrogen, amino, hydroxy, C₁-C₆-alkyl, and amino-C₁-C₆-alkyl;
R¹⁷ is selected from the group consisting of hydrogen, halogen, amino, C₁-C₆-alkyl-NH—, and (C₁-C₆-alkyl)₂N—;
R¹⁸ and R¹⁹ are independently selected from the group consisting of hydrogen, amino, hydroxy, and C₁-C₆-alkyl;
L¹ is selected from the group consisting of carbonyl, a covalent bond and C₁-C₆-alkyldiyl;
L² is selected from the group consisting of carbonyl, —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is selected from the group consisting of a covalent bond, carbonyl, —NH—C(O)—, and —C(O)—NH—; and

A, B, C, and D are independently selected from the group consisting of C₃-C₁₂-cycloalkyl, C₁-C₁₉-heterocyclyl, C₆-C₁₄-aryl, and C₁-C₁₃-heteroaryl.

In one embodiment, the present invention provides a compound of formula (I) as described herein, or a pharmaceutically acceptable salt thereof, wherein:
(a) X is

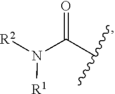

wherein:
   (i) R¹ and R², taken together with the N-atom to which they are attached, form a C₁-C₁₉-heterocycle, wherein the C₁-C₁₉-heterocycle is optionally substituted with one or more, preferably 1, 2, 3 or 4 substituents selected from the group consisting of R¹⁷—C₁-C₆-alkyl-L⁴-, amino, hydroxy, and a group

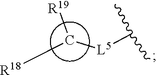

or
(ii) R¹ is selected from the group consisting of:
   (i) hydrogen;
   (ii) C₁-C₆-alkyl optionally substituted with one or more, preferably 1, 2, 3 or 4 substituents selected from the group consisting of C₂-C₆-alkenyl-NH—, C₁-C₆-alkoxy, C₁-C₆-alkoxycarbonyl-NH—, C₂-C₆-alkynyl-NH—, amino, halogen, cyano, hydroxy, and a group

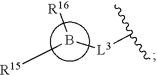

(iii) R¹⁰—C₁-C₆-alkyl-L²-C₁-C₆-alkyl-;
(iv) C₂-C₆-alkynyl;
(v) amino-C₁-C₆-alkoxy; and
(vi) a group

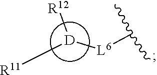

and
R² is hydrogen or C₁-C₆-alkyl; or
(b) X is

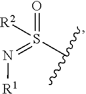

wherein:
   R¹ is selected from the group consisting of:
   (i) C₁-C₆-alkyl optionally substituted with one or more, preferably 1, 2, 3 or 4 substituents selected from the group consisting of C₂-C₆-alkenyl-NH—, C₁-C₆-alkoxy, C₁-C₆-alkoxycarbonyl-NH—, C₂-C₆-alkynyl-NH—, amino, halogen, cyano, hydroxy, and a group

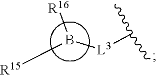

(ii) R¹⁰—C₁-C₆-alkyl-L²-C₁-C₆-alkyl-;
(iii) C₂-C₆-alkynyl; and
(iv) a group

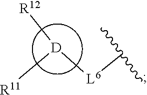

and
R² is C₁-C₆-alkyl;
R³, R⁴, R⁸, and R⁹ are independently selected from the group consisting of hydrogen, halogen, and C₁-C₆-alkyl;
R⁵, R⁶, and R⁷ are independently selected from the group consisting of hydrogen, C₁-C₆-alkyl, C₂-C₆-alkenyl, C₂-C₆-alkynyl, halo-C₂-C₆-alkenyl, CF₃, and a group

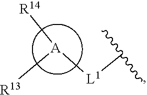

wherein said C₁-C₆-alkyl is optionally substituted with one or more, preferably 1, 2, 3 or 4 substituents independently selected from the group consisting of hydrogen, C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R¹⁰ is selected from the group consisting of amino, C₁-C₆-alkyl-NH—, (C₁-C₆-alkyl)₂N—, C₂-C₆-alkynyl, amino-C₁-C₆-alkyl-NH—, carbamoyl, guanidine, and C₁-C₁₉-heterocyclyl, wherein the C₁-C₁₉-heterocyclyl is optionally substituted with one or two substituents selected from hydroxy and cyano-C₁-C₆-alkyl;
R¹¹ and R¹² are independently selected from the group consisting of amino, hydroxy, hydroxy-C₁-C₁₉-heterocyclyl-C(O)—, and hydroxy-C₁-C₁₉-heterocyclyl-C(O)—NH—;
R¹³ and R¹⁴ are independently selected from the group consisting of hydrogen, halogen, cyano, C₁-C₆-alkyl, amino, hydroxy, amino-C₁-C₆-alkyl, cyano-C₁-C₆-alkyl, hydroxy-C₁-C₆-alkyl, halo-C₁-C₆-alkyl, C₁-C₆-alkoxy, C₆-C₁₄-aryl-C₁-C₆-alkyl-, and C₃-C₁₂-cycloalkyl-C₁-C₆-alkyl-;
R¹⁵ and R¹⁶ are independently selected from the group consisting of hydrogen, amino, hydroxy, C₁-C₆-alkyl, and amino-C₁-C₆-alkyl;
R¹⁷ is selected from the group consisting of hydrogen, halogen, amino, C₁-C₆-alkyl-NH—, and (C₁-C₆-alkyl)₂N—;
R¹⁸ and R¹⁹ are independently selected from the group consisting of hydrogen, amino, hydroxy, and C₁-C₆-alkyl;
L¹ is selected from the group consisting of carbonyl, a covalent bond and C₁-C₆-alkyldiyl;
L² is selected from the group consisting of carbonyl, —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is selected from the group consisting of a covalent bond, carbonyl, —NH—C(O)—, and —C(O)—NH—; and
A, B, C, and D are independently selected from the group consisting of C₃-C₁₂-cycloalkyl, C₁-C₁₉-heterocyclyl, C₆-C₁₄-aryl, and C₁-C₁₃-heteroaryl.

In a preferred embodiment, the present invention provides a compound of formula (I) as described herein, or a pharmaceutically acceptable salt thereof, wherein the compound of formula (I) is a compound of formula (II):

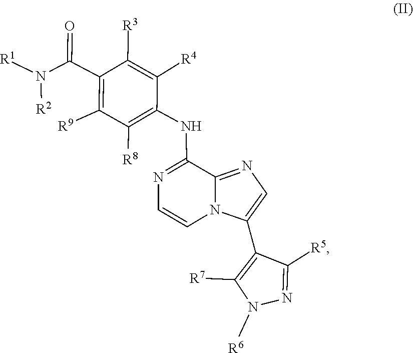

wherein R¹ to R⁹ are as defined herein.

In one embodiment, the present invention provides a compound of formula (I) as described herein, or a pharmaceutically acceptable salt thereof, wherein the compound of formula (I) is a compound of formula (III):

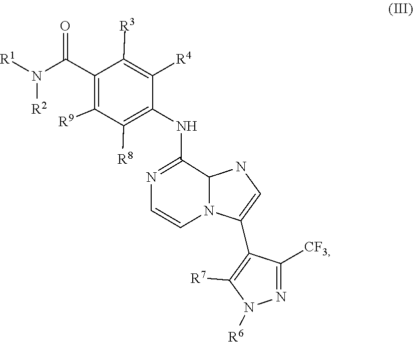

wherein R¹ to R⁴ and R⁶ to R⁹ are as defined herein.

In one embodiment, the present invention provides a compound of formula (I) as described herein, or a pharmaceutically acceptable salt thereof, wherein the compound of formula (I) is a compound of formula (IV):

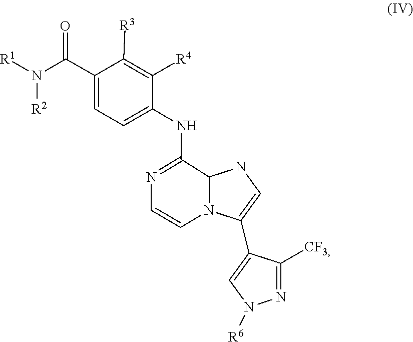

wherein R¹ to R³ and R⁶ are as defined herein.

In one embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, hydroxy, and a group

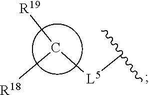

or
(ii) R¹ is selected from the group consisting of:
   (i) hydrogen;
   (ii) C₁-C₆-alkyl optionally substituted with one to two substituents selected from the group consisting of C₂-C₆-alkenyl-NH—, C₁-C₆-alkoxy, C₁-C₆-alkoxycarbonyl-NH—, C₂-C₆-alkynyl-NH—, amino, cyano, hydroxy, and a group

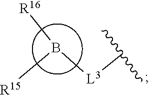

(iii) R¹⁰—C₁-C₆-alkyl-L²-C₁-C₆-alkyl-;
(iv) C₂-C₆-alkynyl;
(v) amino-C₁-C₆-alkoxy; and
(vi) a group

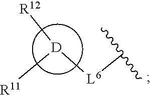

and
R² is hydrogen or C₁-C₆-alkyl.

In a preferred embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, and a group

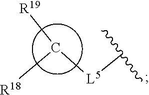

or
(ii) R¹ is selected from the group consisting of:
   (i) C₁-C₆-alkyl substituted with one substituent selected from a group

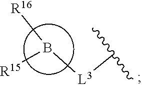

and
(ii) R¹⁰—C₁-C₆-alkyl-L²-C₁-C₆-alkyl-; and
R² is hydrogen.

In a particularly preferred embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring selected from the group consisting of 1-piperidyl, 2,6-diazaspiro[3.3]heptan-2-yl, azetidin-1-yl, and piperazin-1-yl, wherein the C₁-C₁₉-heterocyclic ring is optionally substituted with one to two substituents selected from the group consisting of R¹⁷—CH₂-L⁴-, R¹⁷—CH(CH₃)-L⁴-, R¹⁷—(CH₂)₃-L⁴-, amino, and a group

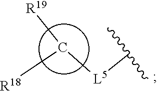

or
(ii) R¹ is selected from the group consisting of:
   (i) ethylydiyl (—(CH₂)₂—) substituted with one substituent selected from a group

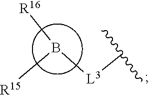

(ii) R¹⁰—(CH₂)₂-L²-(CH₂)₂—;
(iii) R¹⁰—C(CH₃)₂—CH₂-L²-(CH₂)₂—;
(iv) R¹⁰—(CH₂)₂-L²-CH₂—; and
(v) R¹⁰—CH(CH₃)—CH₂-L²-(CH₂)₂—; and
R² is hydrogen.

In a preferred embodiment, the present invention provides a compound of formula (I) as described herein, or a pharmaceutically acceptable salt thereof, wherein R³ is C₁-C₆-alkyl or halogen.

In a particularly preferred embodiment, the present invention provides a compound of formula (I) as described herein, or a pharmaceutically acceptable salt thereof, wherein R³ is selected from the group consisting of methyl, ethyl, and chloro.

In a preferred embodiment, the present invention provides a compound of formula (I) as described herein, or a pharmaceutically acceptable salt thereof, wherein R⁴ is hydrogen.

In one embodiment, the present invention provides a compound of formula (I) as described herein, or a pharmaceutically acceptable salt thereof, wherein R⁵ is halogen, cyano, halo-C₁-C₆-alkyl, C₁-C₆-alkyl-S—, or a group

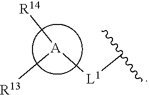

In one embodiment, the present invention provides a compound of formula (I) as described herein, or a pharmaceutically acceptable salt thereof, wherein R⁵ is halo-C₁-C₆-alkyl or a group

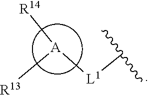

In a preferred embodiment, the present invention provides a compound of formula (I) as described herein, or a pharmaceutically acceptable salt thereof, wherein R⁵ is halo-C₁-C₆-alkyl.

In a particularly preferred embodiment, the present invention provides a compound of formula (I) as described herein, or a pharmaceutically acceptable salt thereof, wherein R⁵ is CF₃ or CHF₂.

In one embodiment, the present invention provides a compound of formula (I) as described herein, or a pharmaceutically acceptable salt thereof, wherein R⁶ is selected from the group consisting of hydrogen, C₁-C₆-alkyl, C₂-C₆-alkenyl, C₂-C₆-alkynyl, halo-C₂-C₆-alkenyl, and a group

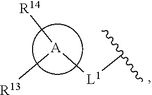

wherein the C₁-C₆-alkyl is optionally substituted with one to two substituents selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃.

In a preferred embodiment, the present invention provides a compound of formula (I) as described herein, or a pharmaceutically acceptable salt thereof, wherein R⁶ is selected from the group consisting of hydrogen, C₁-C₆-alkyl, C₂-C₆-alkenyl, C₂-C₆-alkynyl, halo-C₂-C₆-alkenyl, and a group

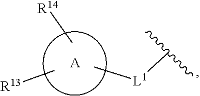

wherein the C₁-C₆-alkyl is optionally substituted with one to two substituents selected from the group consisting of C₁-C₆-alkoxy, halogen, cyano, and CF₃.

In a particularly preferred embodiment, the present invention provides a compound of formula (I) as described herein, or a pharmaceutically acceptable salt thereof, wherein R⁶ is selected from the group consisting of hydrogen, methyl, ethyl, propyl, vinyl, prop-2-ynyl, halo-C₂-C₆-alkenyl, and a group

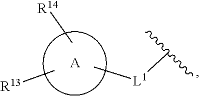

wherein said methyl, ethyl, and propyl are optionally substituted with one to two substituents selected from the group consisting of methoxy, fluoro, cyano, and CF₃.

In one embodiment, the present invention provides a compound of formula (I) as described herein, or a pharmaceutically acceptable salt thereof, wherein R⁷ is hydrogen or C₁-C₆-alkyl.

In a preferred embodiment, the present invention provides a compound of formula (I) as described herein, or a pharmaceutically acceptable salt thereof, wherein R⁷ is hydrogen.

In a preferred embodiment, the present invention provides a compound of formula (I) as described herein, or a pharmaceutically acceptable salt thereof, wherein R⁸ is hydrogen.

In a preferred embodiment, the present invention provides a compound of formula (I) as described herein, or a pharmaceutically acceptable salt thereof, wherein R⁹ is hydrogen or halogen.

In a particularly preferred embodiment, the present invention provides a compound of formula (I) as described herein, or a pharmaceutically acceptable salt thereof, wherein R⁹ is hydrogen or fluoro.

In one embodiment, the present invention provides a compound of formula (I) as described herein, or a pharmaceutically acceptable salt thereof, wherein R¹⁰ is selected from the group consisting of amino, (C₁-C₆-alkyl)₂N—, C₂-C₆-alkynyl; amino-C₁-C₆-alkyl-NH—, carbamoyl, and guanidino.

In a preferred embodiment, the present invention provides a compound of formula (I) as described herein, or a pharmaceutically acceptable salt thereof, wherein R¹⁰ is amino.

In a preferred embodiment, the present invention provides a compound of formula (I) as described herein, or a pharmaceutically acceptable salt thereof, wherein R¹¹ is hydroxy-C₁-C₁₉-heterocyclyl-C(O)—.

In a preferred embodiment, the present invention provides a compound of formula (I) as described herein, or a pharmaceutically acceptable salt thereof, wherein R¹² is selected from the group consisting of amino, and hydroxy-C₁-C₁₉-heterocyclyl-C(O)—NH—.

In one embodiment, the present invention provides a compound of formula (I) as described herein, or a pharmaceutically acceptable salt thereof, wherein R³ is selected from the group consisting of hydrogen, halogen, amino, C₁-C₆-alkyl, C₁-C₆-alkoxy, and C₃-C₁₂-cycloalkyl-C₁-C₆-alkyl-.

In a preferred embodiment, the present invention provides a compound of formula (I) as described herein, or a pharmaceutically acceptable salt thereof, wherein R¹³ is hydrogen.

In one embodiment, the present invention provides a compound of formula (I) as described herein, or a pharmaceutically acceptable salt thereof, wherein R¹⁴ is selected from the group consisting of hydrogen, halogen, and C₁-C₆-alkyl.

In a preferred embodiment, the present invention provides a compound of formula (I) as described herein, or a pharmaceutically acceptable salt thereof, wherein R¹⁴ is hydrogen.

In one embodiment, the present invention provides a compound of formula (I) as described herein, or a pharmaceutically acceptable salt thereof, wherein R¹⁵ is selected from the group consisting of hydrogen, amino, and hydroxy.

In a preferred embodiment, the present invention provides a compound of formula (I) as described herein, or a pharmaceutically acceptable salt thereof, wherein R¹⁵ is hydrogen or amino.

In one embodiment, the present invention provides a compound of formula (I) as described herein, or a pharmaceutically acceptable salt thereof, wherein R¹⁶ is hydrogen or C₁-C₆-alkyl.

In a preferred embodiment, the present invention provides a compound of formula (I) as described herein, or a pharmaceutically acceptable salt thereof, wherein R¹⁶ is hydrogen.

In one embodiment, the present invention provides a compound of formula (I) as described herein, or a pharmaceutically acceptable salt thereof, wherein R¹⁷ is selected from the group consisting of hydrogen, amino, and C₁-C₆-alkyl-NH—.

In a preferred embodiment, the present invention provides a compound of formula (I) as described herein, or a pharmaceutically acceptable salt thereof, wherein R¹⁷ is amino or C₁-C₆-alkyl-NH—.

In a particularly preferred embodiment, the present invention provides a compound of formula (I) as described herein, or a pharmaceutically acceptable salt thereof, wherein R¹⁷ is amino or CH₃NH—.

In a preferred embodiment, the present invention provides a compound of formula (I) as described herein, or a pharmaceutically acceptable salt thereof, wherein R¹⁸ is selected from the group consisting of hydrogen, amino, and hydroxy.

In one embodiment, the present invention provides a compound of formula (I) as described herein, or a pharmaceutically acceptable salt thereof, wherein R¹⁹ is hydrogen or C₁-C₆-alkyl.

In a preferred embodiment, the present invention provides a compound of formula (I) as described herein, or a pharmaceutically acceptable salt thereof, wherein R¹⁹ is hydrogen.

In a preferred embodiment, the present invention provides a compound of formula (I) as described herein, or a pharmaceutically acceptable salt thereof, wherein Lis selected from the group consisting of a covalent bond and C₁-C₆-alkyldiyl.

In a particularly preferred embodiment, the present invention provides a compound of formula (I) as described herein, or a pharmaceutically acceptable salt thereof, wherein L¹ is a covalent bond or —CH₂—.

In a preferred embodiment, the present invention provides a compound of formula (I) as described herein, or a pharmaceutically acceptable salt thereof, wherein L² is —NH—C(O)— or —O—.

In a preferred embodiment, the present invention provides a compound of formula (I) as described herein, or a pharmaceutically acceptable salt thereof, wherein L³ is carbonyl or —C(O)—NH—.

In a preferred embodiment, the present invention provides a compound of formula (I) as described herein, or a pharmaceutically acceptable salt thereof, wherein L⁴ is selected from the group consisting of a covalent bond, carbonyl, and —NH—C(O)—.

In a preferred embodiment, the present invention provides a compound of formula (I) as described herein, or a pharmaceutically acceptable salt thereof, wherein L⁵ is carbonyl or —NH—C(O)—.

In a preferred embodiment, the present invention provides a compound of formula (I) as described herein, or a pharmaceutically acceptable salt thereof, wherein L⁶ is a covalent bond.

In one embodiment, the present invention provides a compound of formula (I) as described herein, or a pharmaceutically acceptable salt thereof, wherein A is selected from the group consisting of C₃-C₁₂-cycloalkyl, C₁-C₁₉-heterocyclyl, and C₁-C₁₃-heteroaryl.

In a preferred embodiment, the present invention provides a compound of formula (I) as described herein, or a pharmaceutically acceptable salt thereof, wherein A is C₃-C₁₂-cycloalkyl.

In a particularly preferred embodiment, the present invention provides a compound of formula (I) as described herein, or a pharmaceutically acceptable salt thereof, wherein A is cyclopropyl or cyclobutyl.

In a preferred embodiment, the present invention provides a compound of formula (I) as described herein, or a pharmaceutically acceptable salt thereof, wherein B is C₁-C₁₉-heterocyclyl or C₃-C₁₂-cycloalkyl.

In a particularly preferred embodiment, the present invention provides a compound of formula (I) as described herein, or a pharmaceutically acceptable salt thereof, wherein B is selected from the group consisting of pyrrolidin-1-yl, pyrrolidin-2-yl, cyclopentyl, cyclobutyl, piperazin-1-yl, and 1-piperidyl.

In a preferred embodiment, the present invention provides a compound of formula (I) as described herein, or a pharmaceutically acceptable salt thereof, wherein C is C₁-C₁₉-heterocyclyl or C₃-C₁₂-cycloalkyl.

In a particularly preferred embodiment, the present invention provides a compound of formula (I) as described herein, or a pharmaceutically acceptable salt thereof, wherein C is selected from the group consisting of pyrrolidin-1-yl, pyrrolidin-2-yl, pyrrolidin-3-yl, 1-piperidyl, 3-piperidyl, cyclobutyl, and cyclopentyl.

In a preferred embodiment, the present invention provides a compound of formula (I) as described herein, or a pharmaceutically acceptable salt thereof, wherein D is C₁-C₁₉-heterocyclyl or C₃-C₁₂-cycloalkyl.

In one embodiment, there is provided a compound of formula (I) as described herein, or a pharmaceutically acceptable salt thereof, wherein R^{1a} is amino-C₁-C₆-alkyl.

In one embodiment, the present invention provides a compound of formula (I) as described herein, or a pharmaceutically acceptable salt thereof, wherein:
(a) X is

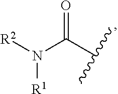

wherein:
   (i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, hydroxy, and a group

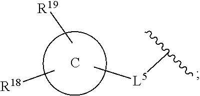

or
(ii) R¹ is selected from the group consisting of:
   (i) hydrogen;
   (ii) C₁-C₆-alkyl optionally substituted with one to two substituents selected from the group consisting of C₂-C₆-alkenyl-NH—, C₁-C₆-alkoxy, C₁-C₆-alkoxycarbonyl-NH—, C₂-C₆-alkynyl-NH—, amino, cyano, hydroxy, and a group

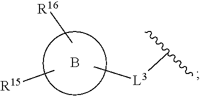

(iii) R¹⁰—C₁-C₆-alkyl-L²-C₁-C₆-alkyl-;
(iv) C₂-C₆-alkynyl;
(v) amino-C₁-C₆-alkoxy; and
(vi) a group

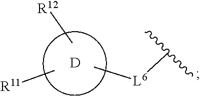

and
R² is hydrogen or C₁-C₆-alkyl; or
(b) X is

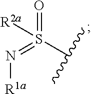

wherein R^{1a} is amino-C₁-C₆-alkyl and R^{2a} is C₁-C₆-alkyl;
R³ is selected from the group consisting of hydrogen, halogen, and C₁-C₆-alkyl;
R⁴ is hydrogen;
R⁵ is halogen, cyano, halo-C₁-C₆-alkyl, C₁-C₆-alkyl-S—, or a group

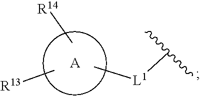

R⁶ is selected from the group consisting of hydrogen, C₁-C₆-alkyl, C₂-C₆-alkenyl, C₂-C₆-alkynyl, halo-C₂-C₆-alkenyl, and a group

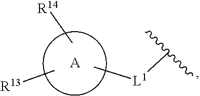

wherein the C₁-C₆-alkyl is optionally substituted with one to two substituents selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R⁷ is hydrogen or C₁-C₆-alkyl;
R⁸ is hydrogen;
R⁹ is selected from the group consisting of hydrogen, halogen, and C₁-C₆-alkyl;
R¹⁰ is selected from the group consisting of amino, (C₁-C₆-alkyl)₂N—, C₂-C₆-alkynyl; amino-C₁-C₆-alkyl-NH—, carbamoyl, and guanidino;
R¹¹ is hydroxy-C₁-C₁₉-heterocyclyl-C(O)—;
R¹² is selected from the group consisting of amino, and hydroxy-C₁-C₁₉-heterocyclyl-C(O)—NH—;
R¹³ is selected from the group consisting of hydrogen, halogen, amino, C₁-C₆-alkyl, C₁-C₆-alkoxy, and C₃-C₁₂-cycloalkyl-C₁-C₆-alkyl-;
R¹⁴ is selected from the group consisting of hydrogen, halogen, and C₁-C₆-alkyl;
R¹⁵ is selected from the group consisting of hydrogen, amino, and hydroxy;
R¹⁶ is hydrogen or C₁-C₆-alkyl;
R¹⁷ is selected from the group consisting of hydrogen, amino, and C₁-C₆-alkyl-NH—;
R¹⁸ is selected from the group consisting of hydrogen, amino, and hydroxy;
R¹⁹ is hydrogen or C₁-C₆-alkyl;
L¹ is selected from the group consisting of carbonyl, a covalent bond and C₁-C₆-alkyldiyl;
L² is selected from the group consisting of —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is a covalent bond;
A is selected from the group consisting of C₃-C₁₂-cycloalkyl, C₁-C₁₉-heterocyclyl, and C₁-C₁₃-heteroaryl;
B is C₁-C₁₉-heterocyclyl or C₃-C₁₂-cycloalkyl;
C is C₁-C₁₉-heterocyclyl or C₃-C₁₂-cycloalkyl; and
D is C₁-C₁₉-heterocyclyl or C₃-C₁₂-cycloalkyl.

In one embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, hydroxy, and a group

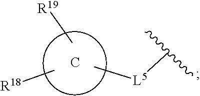

or
(ii) R¹ is selected from the group consisting of:
   (i) hydrogen;
   (ii) C₁-C₆-alkyl optionally substituted with one to two substituents selected from the group consisting of C₂-C₆-alkenyl-NH—, C₁-C₆-alkoxy, C₁-C₆-alkoxycarbonyl-NH—, C₂-C₆-alkynyl-NH—, amino, cyano, hydroxy, and a group

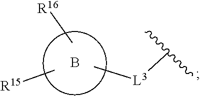

(iii) R¹⁰—C₁-C₆-alkyl-L²-C₁-C₆-alkyl-;
(iv) C₂-C₆-alkynyl;
(v) amino-C₁-C₆-alkoxy; and
(vi) a group

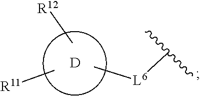

and
R² is hydrogen or C₁-C₆-alkyl;
R³ is selected from the group consisting of hydrogen, halogen, and C₁-C₆-alkyl;
R⁴ is hydrogen;
R⁵ is halo-C₁-C₆-alkyl or a group

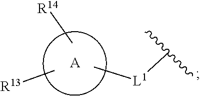

R⁶ is selected from the group consisting of hydrogen, C₁-C₆-alkyl, C₂-C₆-alkenyl, C₂-C₆-alkynyl, halo-C₂-C₆-alkenyl, and a group

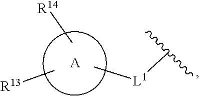

wherein the C₁-C₆-alkyl is optionally substituted with one to two substituents selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R⁷ is hydrogen or C₁-C₆-alkyl;
R⁸ is hydrogen;
R⁹ is selected from the group consisting of hydrogen, halogen, and C₁-C₆-alkyl;
R¹⁰ is selected from the group consisting of amino, (C₁-C₆-alkyl)₂N—, C₂-C₆-alkynyl; amino-C₁-C₆-alkyl-NH—, carbamoyl, and guanidino;
R¹¹ is hydroxy-C₁-C₁₉-heterocyclyl-C(O)—;
R¹² is selected from the group consisting of amino, and hydroxy-C₁-C₁₉-heterocyclyl-C(O)—NH—;
R¹³ is selected from the group consisting of hydrogen, halogen, amino, C₁-C₆-alkyl, C₁-C₆-alkoxy, and C₃-C₁₂-cycloalkyl-C₁-C₆-alkyl-;
R¹⁴ is selected from the group consisting of hydrogen, halogen, and C₁-C₆-alkyl;
R¹⁵ is selected from the group consisting of hydrogen, amino, and hydroxy;
R¹⁶ is hydrogen or C₁-C₆-alkyl;
R¹⁷ is selected from the group consisting of hydrogen, amino, and C₁-C₆-alkyl-NH—;
R¹⁸ is selected from the group consisting of hydrogen, amino, and hydroxy;
R¹⁹ is hydrogen or C₁-C₆-alkyl;
L¹ is selected from the group consisting of carbonyl, a covalent bond and C₁-C₆-alkyldiyl;
L² is selected from the group consisting of —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is a covalent bond;
A is selected from the group consisting of C₃-C₁₂-cycloalkyl, C₁-C₁₉-heterocyclyl, and C₁-C₁₃-heteroaryl;
B is C₁-C₁₉-heterocyclyl or C₃-C₁₂-cycloalkyl;
C is C₁-C₁₉-heterocyclyl or C₃-C₁₂-cycloalkyl; and
D is C₁-C₁₉-heterocyclyl or C₃-C₁₂-cycloalkyl.

In a preferred embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, and a group

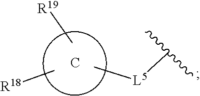

or
(ii) R¹ is selected from the group consisting of:
   (i) C₁-C₆-alkyl substituted with one substituent selected from a group

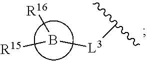

and
(ii) R¹⁰—C₁-C₆-alkyl-L²-C₁-C₆-alkyl-; and
R² is hydrogen;
R³ is C₁-C₆-alkyl or halogen;
R⁴ is hydrogen;
R⁵ is halo-C₁-C₆-alkyl;
R⁶ is selected from the group consisting of hydrogen, C₁-C₆-alkyl, C₂-C₆-alkenyl, C₂-C₆-alkynyl, halo-C₂-C₆-alkenyl, and a group

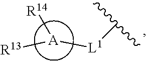

wherein the C₁-C₆-alkyl is optionally substituted with one to two substituents selected from the group consisting of C₁-C₆-alkoxy, halogen, cyano, and CF₃;
R⁷ is hydrogen;
R⁸ is hydrogen;
R⁹ is hydrogen or halogen;
R¹⁰ is amino;
R¹³ is hydrogen;
R¹⁴ is hydrogen;
R¹⁵ is hydrogen or amino;
R¹⁶ is hydrogen;
R¹⁷ is amino or C₁-C₆-alkyl-NH—;
R¹⁸ is selected from the group consisting of hydrogen, amino, and hydroxy;
R¹⁹ is hydrogen;
L¹ is a covalent bond or —CH₂—;
L² is —NH—C(O)— or —O—;
L³ is carbonyl or —C(O)—NH—;
L⁴ is selected from the group consisting of a covalent bond, carbonyl, and —NH—C(O)—;
L⁵ is carbonyl or —NH—C(O)—;
A is C₃-C₁₂-cycloalkyl;
B is C₁-C₁₉-heterocyclyl or C₃-C₁₂-cycloalkyl; and
C is C₁-C₁₉-heterocyclyl or C₃-C₁₂-cycloalkyl.

In a particularly preferred embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring selected from the group consisting of 1-piperidyl, 2,6-diazaspiro[3.3]heptan-2-yl, azetidin-1-yl, and piperazin-1-yl, wherein the C₁-C₁₉-heterocyclic ring is optionally substituted with one to two substituents selected from the group consisting of R¹⁷—CH₂-L⁴-, R¹⁷—CH(CH₃)-L⁴-, R¹⁷—(CH₂)₃-L⁴-, amino, and a group

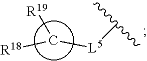

or
(ii) R¹ is selected from the group consisting of:
   (i) ethylydiyl (—(CH₂)₂—) substituted with one substituent selected from a group

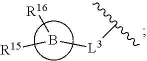

(ii) R¹⁰—(CH₂)₂-L¹-(CH₂)₂—;
(iii) R¹⁰—C(CH₃)₂—CH₂-L¹-(CH₂)₂—;
(iv) R¹⁰—(CH₂)₂-L¹-CH₂—; and
(v) R¹⁰—CH(CH₃)—CH₂-L¹-(CH₂)₂—; and R² is hydrogen;
R³ is selected from the group consisting of methyl, ethyl, and chloro;
R⁴ is hydrogen;
R⁵ is CF₃ or CHF₂;
R⁶ is selected from the group consisting of hydrogen, methyl, ethyl, propyl, vinyl, prop-2-ynyl, halo-C₂-C₆-alkenyl, and a group

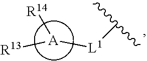

wherein said methyl, ethyl, and propyl are optionally substituted with one to two substituents selected from the group consisting of methoxy, fluoro, cyano, and CF₃;
R⁷ is hydrogen;
R⁸ is hydrogen;
R⁹ is hydrogen or fluoro;
R¹⁰ is amino;
R¹³ is hydrogen;
R¹⁴ is hydrogen;
R¹⁵ is hydrogen or amino;
R¹⁶ is hydrogen;
R¹⁷ is amino or CH₃NH—;
R¹⁸ is selected from the group consisting of hydrogen, amino, and hydroxy;
R¹⁹ is hydrogen;
L¹ is a covalent bond or —CH₂—;
L² is —NH—C(O)— or —O—;
L³ is carbonyl or —C(O)—NH—;
L⁴ is selected from the group consisting of a covalent bond, carbonyl, and —NH—C(O)—;
L⁵ is carbonyl or —NH—C(O)—;
A is cyclopropyl or cyclobutyl;
B is selected from the group consisting of pyrrolidin-1-yl, pyrrolidin-2-yl, cyclopentyl, cyclobutyl, piperazin-1-yl, and 1-piperidyl; and
C is selected from the group consisting of pyrrolidin-1-yl, pyrrolidin-2-yl, pyrrolidin-3-yl, 1-piperidyl, 3-piperidyl, cyclobutyl, and cyclopentyl.

In a particularly preferred embodiment, the group

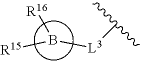

is selected from

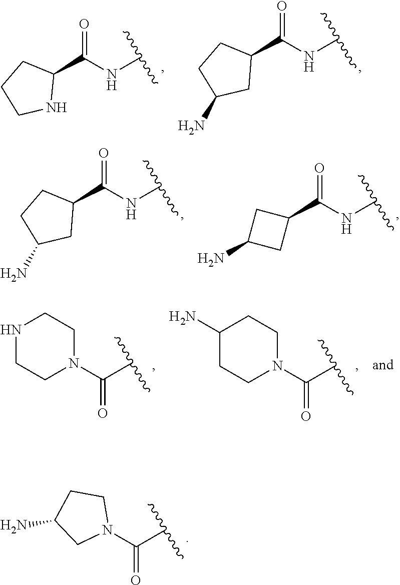

In one embodiment, the present invention provides a compound of formula (I) as described herein, or a pharmaceutically acceptable salt thereof, wherein the compound of formula (I) is selected from:
N-[2-(2-aminoethoxy)ethyl]-2-ethyl-4-[[3-[1-methyl-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(pyrimidin-4-ylmethyl)-3-(trifluoromethyl)pyrazol-4-yl]imidazo[1,2-a]pyrazin-8-yl]amino]benzamide;
(2S)—N-[2-[[4-[[3-[1-(cyanomethyl)-3-(trifluoromethyl)pyrazol-4-yl]imidazo[1,2-a]pyrazin-8-yl]amino]-2-ethylbenzoyl]amino]ethyl]pyrrolidine-2-carboxamide;
4-[[3-[1-(cyanomethyl)-3-(trifluoromethyl)pyrazol-4-yl]imidazo[1,2-a]pyrazin-8-yl]amino]-2-fluoro-6-methyl-N-prop-2-ynylbenzamide;
[2-ethyl-4-[[3-[1-prop-2-ynyl-3-(trifluoromethyl)pyrazol-4-yl]imidazo[1,2-a]pyrazin-8-yl]amino]phenyl]-[4-(piperidine-4-carbonyl)piperazin-1-yl]methanone;
2-amino-1-[4-[2-chloro-4-[[3-[1-(2,2-difluoroethyl)-3-(trifluoromethyl)pyrazol-4-yl]imidazo[1,2-a]pyrazin-8-yl]amino]benzoyl]piperazin-1-yl]ethanone;
N-[3-[4-(3-aminopropylamino)butylamino]propyl]-2-ethyl-4-[[3-[1-(pyridin-3-ylmethyl)-3-(trifluoromethyl)pyrazol-4-yl]imidazo[1,2-a]pyrazin-8-yl]amino]benzamide;
N-[2-(2-aminoethylamino)-2-oxoethyl]-2-chloro-4-[[3-[1-(2-fluoroethyl)-3-(trifluoromethyl)pyrazol-4-yl]imidazo[1,2-a]pyrazin-8-yl]amino]benzamide;
[2-chloro-4-[difluoromethyl-[3-[3-(trifluoromethyl)-1H-pyrazol-4-yl]imidazo[1,2-a]pyrazin-8-yl]amino]phenyl]-piperazin-1-ylmethanone;
2-chloro-4-[[3-[1-(difluoromethyl)-3-(trifluoromethyl)pyrazol-4-yl]imidazo[1,2-a]pyrazin-8-yl]amino]-N-methylbenzamide;
1-[1-[2-chloro-4-[[3-[1-(cyanomethyl)-3-(trifluoromethyl)pyrazol-4-yl]imidazo[1,2-a]pyrazin-8-yl]amino]benzoyl]piperidin-4-yl]-3-[(3S)-pyrrolidin-3-yl]urea;
2-[4-[8-[4-[4-(3-aminocyclobutanecarbonyl)piperazine-1-carbonyl]-3-chloroanilino]imidazo[1,2-a]pyrazin-3-yl]-3-(trifluoromethyl)pyrazol-1-yl]acetonitrile;
N-[3-[[(2S)-2-amino-3-carbamimidamidopropanoyl]amino]propyl]-2-ethyl-4-[[3-[3-(trifluoromethyl)-1H-pyrazol-4-yl]imidazo[1,2-a]pyrazin-8-yl]amino]benzamide;
2-[4-[8-[3-chloro-4-[4-hydroxy-4-(methylaminomethyl)piperidine-1-carbonyl]anilino]imidazo[1,2-a]pyrazin-3-yl]-3-(trifluoromethyl)pyrazol-1-yl]acetonitrile;
2-ethyl-N-methyl-4-[[3-[1-(1H-pyrazol-3-ylmethyl)-3-(trifluoromethyl)pyrazol-4-yl]imidazo[1,2-a]pyrazin-8-yl]amino]benzamide;
N-[2-(2-aminoethoxy)ethyl]-2-methyl-4-[[3-[1-prop-2-ynyl-3-(trifluoromethyl)pyrazol-4-yl]imidazo[1,2-a]pyrazin-8-yl]amino]benzamide;
[4-(3-aminocyclobutanecarbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone;
N-[2-(2-aminoethoxy)ethyl]-4-[[3-[1-(cyanomethyl)-3-(difluoromethyl)pyrazol-4-yl]imidazo[1,2-a]pyrazin-8-yl]amino]-2-ethylbenzamide;
N-[2-(2-aminoethoxy)ethyl]-4-[[3-[3-(difluoromethyl)-1-(methoxymethyl)pyrazol-4-yl]imidazo[1,2-a]pyrazin-8-yl]amino]-2-ethylbenzamide;
N-[2-(2-aminoethoxy)ethyl]-4-[[3-[1-(2,2-difluoroethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(methoxymethyl)-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[3-(trifluoromethyl)-1H-pyrazol-4-yl]imidazo[1,2-a]pyrazin-8-yl]amino]benzamide;
N-[2-(2-aminoethoxy)ethyl]-2-ethyl-4-[[3-[5-methyl-3-(trifluoromethyl)-1H-pyrazol-4-yl]imidazo[1,2-a]pyrazin-8-yl]amino]benzamide;
N-[2-(2-aminoethoxy)ethyl]-2-ethyl-4-[[3-[1-propyl-3-(trifluoromethyl)pyrazol-4-yl]imidazo[1,2-a]pyrazin-8-yl]amino]benzamide;
N-[2-(2-aminoethoxy)ethyl]-4-[[3-[1-cyclopentyl-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-propan-2-yl-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(3-methylbutyl)-3-(trifluoromethyl)pyrazol-4-yl]imidazo[1,2-a]pyrazin-8-yl]amino]benzamide;
N-[2-(2-aminoethoxy)ethyl]-4-[[3-[1-(cyanomethyl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(cyclopropylmethyl)-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-ethyl-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[3-(trifluoromethyl)-1-(3,3,3-trifluoropropyl)pyrazol-4-yl]imidazo[1,2-a]pyrazin-8-yl]amino]benzamide;
N-[2-(2-aminoethoxy)ethyl]-4-[[3-[1-(2-cyanoethyl)-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(2-methoxyethyl)-3-(trifluoromethyl)pyrazol-4-yl]imidazo[1,2-a]pyrazin-8-yl]amino]benzamide;
(2S)—N-[2-[[4-[[3-[1-(2,2-difluoroethyl)-3-(trifluoromethyl)pyrazol-4-yl]imidazo[1,2-a]pyrazin-8-yl]amino]-2-ethylbenzoyl]amino]ethyl]pyrrolidine-2-carboxamide;
(2S)—N-[2-[[4-[[3-[1-(difluoromethyl)-3-(trifluoromethyl)pyrazol-4-yl]imidazo[1,2-a]pyrazin-8-yl]amino]-2-ethylbenzoyl]amino]ethyl]pyrrolidine-2-carboxamide;
4-[[3-[1-(2,2-difluoroethyl)-3-(trifluoromethyl)pyrazol-4-yl]imidazo[1,2-a]pyrazin-8-yl]amino]-2-fluoro-6-methyl-N-prop-2-ynylbenzamide;
4-[[3-[1-[[3-(cyclopropylmethyl)-1,2,4-oxadiazol-5-yl]methyl]-3-(trifluoromethyl)pyrazol-4-yl]imidazo[1,2-a]pyrazin-8-yl]amino]-2-fluoro-6-methyl-N-prop-2-ynylbenzamide;
(2S)—N-[2-[[2-ethyl-4-[[3-[3-(trifluoromethyl)-1H-pyrazol-4-yl]imidazo[1,2-a]pyrazin-8-yl]amino]benzoyl]amino]ethyl]pyrrolidine-2-carboxamide;
2-fluoro-6-methyl-N-prop-2-ynyl-4-[[3-[1-prop-2-ynyl-3-(trifluoromethyl)pyrazol-4-yl]imidazo[1,2-a]pyrazin-8-yl]amino]benzamide;
2-fluoro-6-methyl-4-[[3-[1-prop-2-enyl-3-(trifluoromethyl)pyrazol-4-yl]imidazo[1,2-a]pyrazin-8-yl]amino]-N-prop-2-ynylbenzamide;
2-fluoro-4-[[3-[1-(2-fluoroethyl)-3-(trifluoromethyl)pyrazol-4-yl]imidazo[1,2-a]pyrazin-8-yl]amino]-6-methyl-N-prop-2-ynylbenzamide;
2-fluoro-6-methyl-N-prop-2-ynyl-4-[[3-[3-(trifluoromethyl)-1H-pyrazol-4-yl]imidazo[1,2-a]pyrazin-8-yl]amino]benzamide;
N-[2-(2-amino-2-methylpropoxy)ethyl]-2-ethyl-4-[[3-[1-prop-2-ynyl-3-(trifluoromethyl)pyrazol-4-yl]imidazo[1,2-a]pyrazin-8-yl]amino]benzamide;
N-[2-(2-amino-2-methylpropoxy)ethyl]-2-ethyl-4-[[3-[1-prop-2-enyl-3-(trifluoromethyl)pyrazol-4-yl]imidazo[1,2-a]pyrazin-8-yl]amino]benzamide;
[4-(aminomethyl)piperidin-1-yl]-[2-ethyl-4-[[3-[1-prop-2-ynyl-3-(trifluoromethyl)pyrazol-4-yl]imidazo[1,2-a]pyrazin-8-yl]amino]phenyl]methanone;
N-(3-aminopropyl)-2-ethyl-4-[[3-[1-prop-2-ynyl-3-(trifluoromethyl)pyrazol-4-yl]imidazo[1,2-a]pyrazin-8-yl]amino]benzamide;
N-(3-aminopropyl)-2-ethyl-4-[[3-[1-prop-2-enyl-3-(trifluoromethyl)pyrazol-4-yl]imidazo[1,2-a]pyrazin-8-yl]amino]benzamide;
N-[2-(2-aminoethoxy)ethyl]-4-[[3-[1-(2,2-difluoroethyl)-3-(trifluoromethyl)pyrazol-4-yl]imidazo[1,2-a]pyrazin-8-yl]amino]-2-fluoro-6-methylbenzamide;
N-[2-(2-amino-2-methylpropoxy)ethyl]-4-[[3-[1-(2,2-difluoroethyl)-3-(trifluoromethyl)pyrazol-4-yl]imidazo[1,2-a]pyrazin-8-yl]amino]-2-ethylbenzamide;
N-[2-(2-aminoethoxy)ethyl]-2-fluoro-6-methyl-4-[[3-[1-prop-2-enyl-3-(trifluoromethyl)pyrazol-4-yl]imidazo[1,2-a]pyrazin-8-yl]amino]benzamide;
N-(3-aminopropyl)-4-[[3-[1-(2,2-difluoroethyl)-3-(trifluoromethyl)pyrazol-4-yl]imidazo[1,2-a]pyrazin-8-yl]amino]-2-ethylbenzamide;
[4-(aminomethyl)piperidin-1-yl]-[2-ethyl-4-[[3-[1-prop-2-enyl-3-(trifluoromethyl)pyrazol-4-yl]imidazo[1,2-a]pyrazin-8-yl]amino]phenyl]methanone;
N-[2-(2-amino-2-methylpropoxy)ethyl]-2-ethyl-4-[[3-[1-(2-fluoroethyl)-3-(trifluoromethyl)pyrazol-4-yl]imidazo[1,2-a]pyrazin-8-yl]amino]benzamide;
N-[(2S)-1-aminopropan-2-yl]-2-ethyl-4-[[3-[1-prop-2-ynyl-3-(trifluoromethyl)pyrazol-4-yl]imidazo[1,2-a]pyrazin-8-yl]amino]benzamide;
N-(3-aminopropyl)-2-ethyl-4-[[3-[1-(2-fluoroethyl)-3-(trifluoromethyl)pyrazol-4-yl]imidazo[1,2-a]pyrazin-8-yl]amino]benzamide;
N-[(2S)-1-aminopropan-2-yl]-4-[[3-[1-(2,2-difluoroethyl)-3-(trifluoromethyl)pyrazol-4-yl]imidazo[1,2-a]pyrazin-8-yl]amino]-2-ethylbenzamide;
[4-(aminomethyl)piperidin-1-yl]-[4-[[3-[1-(2,2-difluoroethyl)-3-(trifluoromethyl)pyrazol-4-yl]imidazo[1,2-a]pyrazin-8-yl]amino]-2-ethylphenyl]methanone;
N-[2-(2-aminoethoxy)ethyl]-2-fluoro-6-methyl-4-[[3-[1-prop-2-ynyl-3-(trifluoromethyl)pyrazol-4-yl]imidazo[1,2-a]pyrazin-8-yl]amino]benzamide;
N-[2-(2-amino-2-methylpropoxy)ethyl]-4-[[3-[1-(cyanomethyl)-3-(trifluoromethyl)pyrazol-4-yl]imidazo[1,2-a]pyrazin-8-yl]amino]-2-ethylbenzamide;
N-[2-(2-aminoethoxy)ethyl]-2-fluoro-4-[[3-[1-(2-fluoroethyl)-3-(trifluoromethyl)pyrazol-4-yl]imidazo[1,2-a]pyrazin-8-yl]amino]-6-methylbenzamide;
[4-(aminomethyl)piperidin-1-yl]-[2-ethyl-4-[[3-[1-(2-fluoroethyl)-3-(trifluoromethyl)pyrazol-4-yl]imidazo[1,2-a]pyrazin-8-yl]amino]phenyl]methanone;
N-[(2S)-1-aminopropan-2-yl]-2-ethyl-4-[[3-[1-prop-2-enyl-3-(trifluoromethyl)pyrazol-4-yl]imidazo[1,2-a]pyrazin-8-yl]amino]benzamide;
[2-ethyl-4-[[3-[1-prop-2-enyl-3-(trifluoromethyl)pyrazol-4-yl]imidazo[1,2-a]pyrazin-8-yl]amino]phenyl]-[4-(piperidine-4-carbonyl)piperazin-1-yl]methanone;
N-[(2S)-1-aminopropan-2-yl]-2-ethyl-4-[[3-[1-(2-fluoroethyl)-3-(trifluoromethyl)pyrazol-4-yl]imidazo[1,2-a]pyrazin-8-yl]amino]benzamide;
2-fluoro-6-methyl-4-[[3-[1-prop-2-enyl-3-(trifluoromethyl)pyrazol-4-yl]imidazo[1,2-a]pyrazin-8-yl]amino]-N-[3-(prop-2-ynylamino)propyl]benzamide;
[2-fluoro-6-methyl-4-[[3-[1-prop-2-ynyl-3-(trifluoromethyl)pyrazol-4-yl]imidazo[1,2-a]pyrazin-8-yl]amino]phenyl]-piperazin-1-ylmethanone;
4-[[3-[1-(2,2-difluoroethyl)-3-(trifluoromethyl)pyrazol-4-yl]imidazo[1,2-a]pyrazin-8-yl]amino]-2-fluoro-6-methyl-N-[3-(prop-2-ynylamino)propyl]benzamide;
[2-fluoro-6-methyl-4-[[3-[1-prop-2-enyl-3-(trifluoromethyl)pyrazol-4-yl]imidazo[1,2-a]pyrazin-8-yl]amino]phenyl]-piperazin-1-ylmethanone;
[4-[[3-[1-(2,2-difluoroethyl)-3-(trifluoromethyl)pyrazol-4-yl]imidazo[1,2-a]pyrazin-8-yl]amino]-2-ethylphenyl]-[4-(piperidine-4-carbonyl)piperazin-1-yl]methanone;
[2-ethyl-4-[[3-[1-(2-fluoroethyl)-3-(trifluoromethyl)pyrazol-4-yl]imidazo[1,2-a]pyrazin-8-yl]amino]phenyl]-[4-(piperidine-4-carbonyl)piperazin-1-yl]methanone;
2-fluoro-6-methyl-N-[3-(prop-2-ynylamino)propyl]-4-[[3-[1-prop-2-ynyl-3-(trifluoromethyl)pyrazol-4-yl]imidazo[1,2-a]pyrazin-8-yl]amino]benzamide;
[2-fluoro-4-[[3-[1-(2-fluoroethyl)-3-(trifluoromethyl)pyrazol-4-yl]imidazo[1,2-a]pyrazin-8-yl]amino]-6-methylphenyl]-piperazin-1-ylmethanone;
2-fluoro-4-[[3-[1-(2-fluoroethyl)-3-(trifluoromethyl)pyrazol-4-yl]imidazo[1,2-a]pyrazin-8-yl]amino]-6-methyl-N-[3-(prop-2-ynylamino)propyl]benzamide;
N-[2-(2-aminoethoxy)ethyl]-4-[[3-[1-(cyanomethyl)-3-(trifluoromethyl)pyrazol-4-yl]imidazo[1,2-a]pyrazin-8-yl]amino]-2-fluoro-6-methylbenzamide;
4-[[3-[1-(cyanomethyl)-3-(trifluoromethyl)pyrazol-4-yl]imidazo[1,2-a]pyrazin-8-yl]amino]-2-fluoro-6-methyl-N-[3-(prop-2-ynylamino)propyl]benzamide;
2-[4-[8-[3-fluoro-5-methyl-4-(piperazine-1-carbonyl)anilino]imidazo[1,2-a]pyrazin-3-yl]-3-(trifluoromethyl)pyrazol-1-yl]acetonitrile;
2-amino-1-[4-[2-chloro-4-[[3-[1-(difluoromethyl)-3-(trifluoromethyl)pyrazol-4-yl]imidazo[1,2-a]pyrazin-8-yl]amino]benzoyl]piperazin-1-yl]ethanone;
[2-chloro-4-[[3-[1-(difluoromethyl)-3-(trifluoromethyl)pyrazol-4-yl]imidazo[1,2-a]pyrazin-8-yl]amino]phenyl]-(2,6-diazaspiro[3.3]heptan-2-yl)methanone;
N-[2-(2-aminoethoxy)ethyl]-2-fluoro-6-methyl-4-[[3-[3-(trifluoromethyl)-1H-pyrazol-4-yl]imidazo[1,2-a]pyrazin-8-yl]amino]benzamide;
N-(5-aminopentyl)-2-fluoro-6-methyl-4-[[3-[3-(trifluoromethyl)-1H-pyrazol-4-yl]imidazo[1,2-a]pyrazin-8-yl]amino]benzamide;
N-[3-[4-(3-aminopropylamino)butylamino]propyl]-2-ethyl-4-[[3-[1-[(2-methyl-1,3-thiazol-4-yl)methyl]-3-(trifluoromethyl)pyrazol-4-yl]imidazo[1,2-a]pyrazin-8-yl]amino]benzamide;
N-[(2R)-1-aminopropan-2-yl]-2-fluoro-6-methyl-4-[[3-[3-(trifluoromethyl)-1H-pyrazol-4-yl]imidazo[1,2-a]pyrazin-8-yl]amino]benzamide;
N-[3-[4-(3-aminopropylamino)butylamino]propyl]-2-ethyl-4-[[3-[1-[(1-methyl)imidazol-2-yl)methyl]-3-(trifluoromethyl)pyrazol-4-yl]imidazo[1,2-a]pyrazin-8-yl]amino]benzamide;
2-fluoro-6-methyl-N-[3-(prop-2-ynylamino)propyl]-4-[[3-[3-(trifluoromethyl)-1H-pyrazol-4-yl]imidazo[1,2-a]pyrazin-8-yl]amino]benzamide;
[2-fluoro-6-methyl-4-[[3-[3-(trifluoromethyl)-1H-pyrazol-4-yl]imidazo[1,2-a]pyrazin-8-yl]amino]phenyl]-piperazin-1-ylmethanone;
N-(3-amino-1-bicyclo[1.1.1]pentanyl)-2-chloro-4-[[3-[1-(2,2-difluoroethyl)-3-(trifluoromethyl)pyrazol-4-yl]imidazo[1,2-a]pyrazin-8-yl]amino]benzamide;
N-(3-amino-1-bicyclo[1.1.1]pentanyl)-2-chloro-4-[[3-[1-(cyanomethyl)-3-(trifluoromethyl)pyrazol-4-yl]imidazo[1,2-a]pyrazin-8-yl]amino]benzamide;
N-[2-(2-aminoethylamino)-2-oxoethyl]-4-[[3-[1-but-2-ynyl-3-(trifluoromethyl)pyrazol-4-yl]imidazo[1,2-a]pyrazin-8-yl]amino]-2-chlorobenzamide;
N-[(2R)-1-(4-aminopiperidin-1-yl)-1-oxopropan-2-yl]-2-chloro-4-[[3-[3-(trifluoromethyl)-1H-pyrazol-4-yl]imidazo[1,2-a]pyrazin-8-yl]amino]benzamide;
2-chloro-N-[(2R)-1-oxo-1-piperazin-1-ylpropan-2-yl]-4-[[3-[3-(trifluoromethyl)-1H-pyrazol-4-yl]imidazo[1,2-a]pyrazin-8-yl]amino]benzamide;
N-[(2R)-1-[(3-aminocyclobutyl)amino]-1-oxopropan-2-yl]-2-chloro-4-[[3-[3-(trifluoromethyl)-1H-pyrazol-4-yl]imidazo[1,2-a]pyrazin-8-yl]amino]benzamide;
2-chloro-N-[(2R)-1-oxo-1-[[(3R)-pyrrolidin-3-yl]amino]propan-2-yl]-4-[[3-[3-(trifluoromethyl)-1H-pyrazol-4-yl]imidazo[1,2-a]pyrazin-8-yl]amino]benzamide;
N-[(2R)-1-[(3S)-3-aminopyrrolidin-1-yl]-1-oxopropan-2-yl]-2-chloro-4-[[3-[3-(trifluoromethyl)-1H-pyrazol-4-yl]imidazo[1,2-a]pyrazin-8-yl]amino]benzamide;
N-[(2R)-1-(2-aminoethylamino)-1-oxopropan-2-yl]-2-chloro-4-[[3-[3-(trifluoromethyl)-1H-pyrazol-4-yl]imidazo[1,2-a]pyrazin-8-yl]amino]benzamide;
2-chloro-N-[(2R)-1-oxo-1-[[(3S)-pyrrolidin-3-yl]amino]propan-2-yl]-4-[[3-[3-(trifluoromethyl)-1H-pyrazol-4-yl]imidazo[1,2-a]pyrazin-8-yl]amino]benzamide;
N-[(2R)-1-[(3R)-3-aminopyrrolidin-1-yl]-1-oxopropan-2-yl]-2-chloro-4-[[3-[3-(trifluoromethyl)-1H-pyrazol-4-yl]imidazo[1,2-a]pyrazin-8-yl]amino]benzamide;
2-chloro-N-[(2R)-1-[2-(dimethylamino)ethylamino]-1-oxopropan-2-yl]-4-[[3-[3-(trifluoromethyl)-1H-pyrazol-4-yl]imidazo[1,2-a]pyrazin-8-yl]amino]benzamide;
N-[(2R)-1-(3-aminoazetidin-1-yl)-1-oxopropan-2-yl]-2-chloro-4-[[3-[3-(trifluoromethyl)-1H-pyrazol-4-yl]imidazo[1,2-a]pyrazin-8-yl]amino]benzamide;
2-[4-[8-[4-(3-aminoazetidine-1-carbonyl)-3-chloroanilino]imidazo[1,2-a]pyrazin-3-yl]-3-(trifluoromethyl)pyrazol-1-yl]acetonitrile;
(3-aminoazetidin-1-yl)-[2-chloro-4-[[3-[1-(2,2-difluoroethyl)-3-(trifluoromethyl)pyrazol-4-yl]imidazo[1,2-a]pyrazin-8-yl]amino]phenyl]methanone;
2-[4-[8-[3-chloro-4-(2,6-diazaspiro[3.3]heptane-2-carbonyl)anilino]imidazo[1,2-a]pyrazin-3-yl]-3-(trifluoromethyl)pyrazol-1-yl]acetonitrile;
(4-aminopiperidin-1-yl)-[2-chloro-4-[[3-[1-(2,2-difluoroethyl)-3-(trifluoromethyl)pyrazol-4-yl]imidazo[1,2-a]pyrazin-8-yl]amino]phenyl]methanone;
[2-chloro-4-[[3-[1-(difluoromethyl)-3-(trifluoromethyl)pyrazol-4-yl]imidazo[1,2-a]pyrazin-8-yl]amino]phenyl]-[4-[(2S)-pyrrolidine-2-carbonyl]piperazin-1-yl]methanone;
N-(3-amino-1-bicyclo[1.1.1]pentanyl)-2-chloro-4-[[3-[3-(trifluoromethyl)-1H-pyrazol-4-yl]imidazo[1,2-a]pyrazin-8-yl]amino]benzamide;
N-[(2R)-1-aminopropan-2-yl]-2-ethyl-4-[[3-[1-prop-2-ynyl-3-(trifluoromethyl)pyrazol-4-yl]imidazo[1,2-a]pyrazin-8-yl]amino]benzamide;
N-[(2R)-1-aminopropan-2-yl]-4-[[3-[1-(2,2-difluoroethyl)-3-(trifluoromethyl)pyrazol-4-yl]imidazo[1,2-a]pyrazin-8-yl]amino]-2-ethylbenzamide;
N-[(2R)-1-aminopropan-2-yl]-2-ethyl-4-[[3-[1-prop-2-enyl-3-(trifluoromethyl)pyrazol-4-yl]imidazo[1,2-a]pyrazin-8-yl]amino]benzamide;
N-[(2R)-1-aminopropan-2-yl]-4-[[3-[1-(cyanomethyl)-3-(trifluoromethyl)pyrazol-4-yl]imidazo[1,2-a]pyrazin-8-yl]amino]-2-ethylbenzamide;
N-[(2R)-1-aminopropan-2-yl]-2-ethyl-4-[[3-[1-(2-fluoroethyl)-3-(trifluoromethyl)pyrazol-4-yl]imidazo[1,2-a]pyrazin-8-yl]amino]benzamide;
[2-chloro-4-[[3-[3-(trifluoromethyl)-1H-pyrazol-4-yl]imidazo[1,2-a]pyrazin-8-yl]amino]phenyl]-(2,6-diazaspiro[3.3]heptan-2-yl)methanone;
N,2-diethyl-4-[[3-[1-prop-2-ynyl-3-(trifluoromethyl)pyrazol-4-yl]imidazo[1,2-a]pyrazin-8-yl]amino]benzamide;
4-[[3-[1-(cyanomethyl)-3-(trifluoromethyl)pyrazol-4-yl]imidazo[1,2-a]pyrazin-8-yl]amino]-N,2-diethylbenzamide;
N,2-diethyl-4-[[3-[1-(2-fluoroethyl)-3-(trifluoromethyl)pyrazol-4-yl]imidazo[1,2-a]pyrazin-8-yl]amino]benzamide;
[2-chloro-4-[[3-[1-(2,2-difluoroethyl)-3-(trifluoromethyl)pyrazol-4-yl]imidazo[1,2-a]pyrazin-8-yl]amino]phenyl]-[4-[(2S)-pyrrolidine-2-carbonyl]piperazin-1-yl]methanone;
N-[2-(2-aminoethylamino)-2-oxoethyl]-2-chloro-4-[[3-[1-prop-2-ynyl-3-(trifluoromethyl)pyrazol-4-yl]imidazo[1,2-a]pyrazin-8-yl]amino]benzamide;
N-[2-(2-aminoethylamino)-2-oxoethyl]-2-chloro-4-[[3-[1-(2,2-difluoroethyl)-3-(trifluoromethyl)pyrazol-4-yl]imidazo[1,2-a]pyrazin-8-yl]amino]benzamide;
[2-chloro-4-[[3-[1-[(5-methyl-1H-pyrazol-3-yl)methyl]-3-(trifluoromethyl)pyrazol-4-yl]imidazo[1,2-a]pyrazin-8-yl]amino]phenyl]-piperazin-1-ylmethanone;
N-[2-(2-aminoethylamino)-2-oxoethyl]-2-chloro-4-[[3-[1-(cyanomethyl)-3-(trifluoromethyl)pyrazol-4-yl]imidazo[1,2-a]pyrazin-8-yl]amino]benzamide;
[2-chloro-4-[[3-[1-(2,2-difluoroethyl)-3-(trifluoromethyl)pyrazol-4-yl]imidazo[1,2-a]pyrazin-8-yl]amino]phenyl]-piperazin-1-ylmethanone;
N-[2-(2-aminoethoxy)ethyl]-4-[[3-[3-(difluoromethyl)-1-prop-2-enylpyrazol-4-yl]imidazo[1,2-a]pyrazin-8-yl]amino]-2-ethylbenzamide;
N-[2-(2-aminoethoxy)ethyl]-4-[[3-[1-(2,2-difluoroethyl)-3-(difluoromethyl)pyrazol-4-yl]imidazo[1,2-a]pyrazin-8-yl]amino]-2-ethylbenzamide;
N-[2-(2-aminoethoxy)ethyl]-4-[[3-[3-(difluoromethyl)-1-prop-2-ynylpyrazol-4-yl]imidazo[1,2-a]pyrazin-8-yl]amino]-2-ethylbenzamide;
2-chloro-4-[[3-[1-(2,2-difluoroethyl)-3-(trifluoromethyl)pyrazol-4-yl]imidazo[1,2-a]pyrazin-8-yl]amino]-N-methylbenzamide;
2-chloro-N-[2-oxo-2-[[(3S)-pyrrolidin-3-yl]amino]ethyl]-4-[[3-[3-(trifluoromethyl)-1H-pyrazol-4-yl]imidazo[1,2-a]pyrazin-8-yl]amino]benzamide;
N-[(2S)-1-(4-aminopiperidin-1-yl)-1-oxopropan-2-yl]-2-chloro-4-[[3-[3-(trifluoromethyl)-1H-pyrazol-4-yl]imidazo[1,2-a]pyrazin-8-yl]amino]benzamide;
2-chloro-N-[(2S)-1-oxo-1-[[(3S)-pyrrolidin-3-yl]amino]propan-2-yl]-4-[[3-[3-(trifluoromethyl)-1H-pyrazol-4-yl]imidazo[1,2-a]pyrazin-8-yl]amino]benzamide;
tert-butyl N-[(2R)-2-[[2-ethyl-4-[[3-[1-prop-2-ynyl-3-(trifluoromethyl)pyrazol-4-yl]imidazo[1,2-a]pyrazin-8-yl]amino]benzoyl]amino]propyl]carbamate;
tert-butyl N-[(2R)-2-[[2-ethyl-4-[[3-[1-prop-2-enyl-3-(trifluoromethyl)pyrazol-4-yl]imidazo[1,2-a]pyrazin-8-yl]amino]benzoyl]amino]propyl]carbamate;
tert-butyl N-[(2R)-2-[[4-[[3-[1-(2,2-difluoroethyl)-3-(trifluoromethyl)pyrazol-4-yl]imidazo[1,2-a]pyrazin-8-yl]amino]-2-ethylbenzoyl]amino]propyl]carbamate;
2-amino-1-[4-[2-chloro-4-[[3-[3-(trifluoromethyl)-1H-pyrazol-4-yl]imidazo[1,2-a]pyrazin-8-yl]amino]benzoyl]piperazin-1-yl]ethanone;
2-[4-[8-[4-[4-(2-aminoacetyl)piperazine-1-carbonyl]-3-chloroanilino]imidazo[1,2-a]pyrazin-3-yl]-3-(trifluoromethyl)pyrazol-1-yl]acetonitrile;
2-(azetidin-3-yl)-1-[4-[2-chloro-4-[[3-[3-(trifluoromethyl)-1H-pyrazol-4-yl]imidazo[1,2-a]pyrazin-8-yl]amino]benzoyl]piperazin-1-yl]ethanone;
[2-chloro-4-[[3-[1-[(1-methylpyrazol-4-yl)methyl]-3-(trifluoromethyl)pyrazol-4-yl]imidazo[1,2-a]pyrazin-8-yl]amino]phenyl]-piperazin-1-ylmethanone;
[2-chloro-4-[[3-[1-(pyridazin-3-ylmethyl)-3-(trifluoromethyl)pyrazol-4-yl]imidazo[1,2-a]pyrazin-8-yl]amino]phenyl]-piperazin-1-ylmethanone;
N-[2-(4-aminopiperidin-1-yl)-2-oxoethyl]-2-chloro-4-[[3-[3-(trifluoromethyl)-1H-pyrazol-4-yl]imidazo[1,2-a]pyrazin-8-yl]amino]benzamide;
N-[2-[(3R)-3-aminopyrrolidin-1-yl]-2-oxoethyl]-2-chloro-4-[[3-[3-(trifluoromethyl)-1H-pyrazol-4-yl]imidazo[1,2-a]pyrazin-8-yl]amino]benzamide;
N-[2-[(3-aminocyclobutyl)amino]-2-oxoethyl]-2-chloro-4-[[3-[3-(trifluoromethyl)-1H-pyrazol-4-yl]imidazo[1,2-a]pyrazin-8-yl]amino]benzamide;
2-chloro-N-[2-oxo-2-[[(3R)-pyrrolidin-3-yl]amino]ethyl]-4-[[3-[3-(trifluoromethyl)-1H-pyrazol-4-yl]imidazo[1,2-a]pyrazin-8-yl]amino]benzamide;
2-chloro-N-[(2S)-1-oxo-1-piperazin-1-ylpropan-2-yl]-4-[[3-[3-(trifluoromethyl)-1H-pyrazol-4-yl]imidazo[1,2-a]pyrazin-8-yl]amino]benzamide;
N-[2-(3-aminoazetidin-1-yl)-2-oxoethyl]-2-chloro-4-[[3-[3-(trifluoromethyl)-1H-pyrazol-4-yl]imidazo[1,2-a]pyrazin-8-yl]amino]benzamide;
N-[1-[2-chloro-4-[[3-[1-(cyanomethyl)-3-(trifluoromethyl)pyrazol-4-yl]imidazo[1,2-a]pyrazin-8-yl]amino]benzoyl]piperidin-4-yl]piperidine-4-carboxamide;
(2S,4R)—N-[1-[2-chloro-4-[[3-[1-(cyanomethyl)-3-(trifluoromethyl)pyrazol-4-yl]imidazo[1,2-a]pyrazin-8-yl]amino]benzoyl]piperidin-4-yl]-4-hydroxypyrrolidine-2-carboxamide;
1-[1-[2-chloro-4-[[3-[3-(trifluoromethyl)-1H-pyrazol-4-yl]imidazo[1,2-a]pyrazin-8-yl]amino]benzoyl]piperidin-4-yl]-3-[(3S)-pyrrolidin-3-yl]urea;
N-[(2R)-1-aminopropan-2-yl]-2-ethyl-4-[[3-[3-(trifluoromethyl)-1H-pyrazol-4-yl]imidazo[1,2-a]pyrazin-8-yl]amino]benzamide;
N-[2-[(2S)-2-aminopropoxy]ethyl]-2-ethyl-4-[[3-[1-prop-2-enyl-3-(trifluoromethyl)pyrazol-4-yl]imidazo[1,2-a]pyrazin-8-yl]amino]benzamide;
N-[2-[(2S)-2-aminopropoxy]ethyl]-2-ethyl-4-[[3-[1-prop-2-ynyl-3-(trifluoromethyl)pyrazol-4-yl]imidazo[1,2-a]pyrazin-8-yl]amino]benzamide;
N-[2-[(2R)-2-aminopropoxy]ethyl]-2-ethyl-4-[[3-[1-prop-2-enyl-3-(trifluoromethyl)pyrazol-4-yl]imidazo[1,2-a]pyrazin-8-yl]amino]benzamide;
N-[2-[(2R)-2-aminopropoxy]ethyl]-2-ethyl-4-[[3-[1-prop-2-ynyl-3-(trifluoromethyl)pyrazol-4-yl]imidazo[1,2-a]pyrazin-8-yl]amino]benzamide;
N-[2-[(2R)-2-aminopropoxy]ethyl]-2-ethyl-4-[[3-[1-(2-fluoroethyl)-3-(trifluoromethyl)pyrazol-4-yl]imidazo[1,2-a]pyrazin-8-yl]amino]benzamide;
N-[2-[(2R)-2-aminopropoxy]ethyl]-4-[[3-[1-(2,2-difluoroethyl)-3-(trifluoromethyl)pyrazol-4-yl]imidazo[1,2-a]pyrazin-8-yl]amino]-2-ethylbenzamide;
N-[2-[(2S)-2-aminopropoxy]ethyl]-4-[[3-[1-(cyanomethyl)-3-(trifluoromethyl)pyrazol-4-yl]imidazo[1,2-a]pyrazin-8-yl]amino]-2-ethylbenzamide;
N-[2-[(2S)-2-aminopropoxy]ethyl]-4-[[3-[1-(2,2-difluoroethyl)-3-(trifluoromethyl)pyrazol-4-yl]imidazo[1,2-a]pyrazin-8-yl]amino]-2-ethylbenzamide;
(2S,4R)—N-[1-[2-chloro-4-[[3-[3-(trifluoromethyl)-1H-pyrazol-4-yl]imidazo[1,2-a]pyrazin-8-yl]amino]benzoyl]piperidin-4-yl]-4-hydroxypyrrolidine-2-carboxamide;
N-[3-[4-(3-aminopropylamino)butylamino]propyl]-2-ethyl-4-[[3-[1-prop-2-ynyl-3-(trifluoromethyl)pyrazol-4-yl]imidazo[1,2-a]pyrazin-8-yl]amino]benzamide;
N-[2-[(2R)-2-aminopropoxy]ethyl]-4-[[3-[1-(cyanomethyl)-3-(trifluoromethyl)pyrazol-4-yl]imidazo[1,2-a]pyrazin-8-yl]amino]-2-ethylbenzamide;
N-[1-[2-chloro-4-[[3-[3-(trifluoromethyl)-1H-pyrazol-4-yl]imidazo[1,2-a]pyrazin-8-yl]amino]benzoyl]piperidin-4-yl]piperidine-4-carboxamide;
N-[2-[(2S)-2-aminopropoxy]ethyl]-2-ethyl-4-[[3-[1-(2-fluoroethyl)-3-(trifluoromethyl)pyrazol-4-yl]imidazo[1,2-a]pyrazin-8-yl]amino]benzamide;
[2-chloro-4-[[3-[1-[(6-methoxypyridazin-3-yl)methyl]-3-(trifluoromethyl)pyrazol-4-yl]imidazo[1,2-a]pyrazin-8-yl]amino]phenyl]-piperazin-1-ylmethanone;
[2-chloro-4-[[3-[1-[(5-methylpyrimidin-2-yl)methyl]-3-(trifluoromethyl)pyrazol-4-yl]imidazo[1,2-a]pyrazin-8-yl]amino]phenyl]-piperazin-1-ylmethanone;
[2-chloro-4-[[3-[1-[(4-methylpyrimidin-2-yl)methyl]-3-(trifluoromethyl)pyrazol-4-yl]imidazo[1,2-a]pyrazin-8-yl]amino]phenyl]-piperazin-1-ylmethanone;
2-chloro-N-(2-oxo-2-piperazin-1-ylethyl)-4-[[3-[3-(trifluoromethyl)-1H-pyrazol-4-yl]imidazo[1,2-a]pyrazin-8-yl]amino]benzamide;
[2-chloro-4-[[3-[1-[(6-methylpyridazin-3-yl)methyl]-3-(trifluoromethyl)pyrazol-4-yl]imidazo[1,2-a]pyrazin-8-yl]amino]phenyl]-piperazin-1-ylmethanone;
N-[2-[(3S)-3-aminopyrrolidin-1-yl]-2-oxoethyl]-2-chloro-4-[[3-[3-(trifluoromethyl)-1H-pyrazol-4-yl]imidazo[1,2-a]pyrazin-8-yl]amino]benzamide;
2-chloro-N-[2-[2-(dimethylamino)ethylamino]-2-oxoethyl]-4-[[3-[3-(trifluoromethyl)-1H-pyrazol-4-yl]imidazo[1,2-a]pyrazin-8-yl]amino]benzamide;
N-[2-(2-aminoethylamino)-2-oxoethyl]-2-chloro-4-[[3-[3-(trifluoromethyl)-1H-pyrazol-4-yl]imidazo[1,2-a]pyrazin-8-yl]amino]benzamide;
(3-aminoazetidin-1-yl)-[2-chloro-4-[[3-[3-(trifluoromethyl)-1H-pyrazol-4-yl]imidazo[1,2-a]pyrazin-8-yl]amino]phenyl]methanone;
1-[1-[2-chloro-4-[[3-[3-(trifluoromethyl)-1H-pyrazol-4-yl]imidazo[1,2-a]pyrazin-8-yl]amino]benzoyl]azetidin-3-yl]-3-[(3R)-pyrrolidin-3-yl]urea;
[2-chloro-4-[[3-[1-(pyrimidin-2-ylmethyl)-3-(trifluoromethyl)pyrazol-4-yl]imidazo[1,2-a]pyrazin-8-yl]amino]phenyl]-piperazin-1-ylmethanone;
N-[1-[2-chloro-4-[[3-[1-(cyanomethyl)-3-(trifluoromethyl)pyrazol-4-yl]imidazo[1,2-a]pyrazin-8-yl]amino]benzoyl]azetidin-3-yl]piperidine-4-carboxamide;
1-[1-[2-chloro-4-[[3-[1-(cyanomethyl)-3-(trifluoromethyl)pyrazol-4-yl]imidazo[1,2-a]pyrazin-8-yl]amino]benzoyl]azetidin-3-yl]-3-[(3R)-pyrrolidin-3-yl]urea;
1-[1-[2-chloro-4-[[3-[1-(cyanomethyl)-3-(trifluoromethyl)pyrazol-4-yl]imidazo[1,2-a]pyrazin-8-yl]amino]benzoyl]azetidin-3-yl]-3-[(3S)-pyrrolidin-3-yl]urea;
(2S,4R)—N-[1-[2-chloro-4-[[3-[1-(cyanomethyl)-3-(trifluoromethyl)pyrazol-4-yl]imidazo[1,2-a]pyrazin-8-yl]amino]benzoyl]azetidin-3-yl]-4-hydroxypyrrolidine-2-carboxamide;
N-[2-(2-aminoethoxy)ethyl]-2-ethyl-4-[[3-[1-prop-2-ynyl-3-(trifluoromethyl)pyrazol-4-yl]imidazo[1,2-a]pyrazin-8-yl]amino]benzamide;
(2S,4R)—N-[1-[2-chloro-4-[[3-[3-(trifluoromethyl)-1H-pyrazol-4-yl]imidazo[1,2-a]pyrazin-8-yl]amino]benzoyl]azetidin-3-yl]-4-hydroxypyrrolidine-2-carboxamide;
N-[2-(2-amino-2-methylpropoxy)ethyl]-2-ethyl-4-[[3-[3-(trifluoromethyl)-1H-pyrazol-4-yl]imidazo[1,2-a]pyrazin-8-yl]amino]benzamide;
[2-chloro-4-[[3-[1-(1H-pyrazol-3-ylmethyl)-3-(trifluoromethyl)pyrazol-4-yl]imidazo[1,2-a]pyrazin-8-yl]amino]phenyl]-piperazin-1-ylmethanone;
2-[4-[8-[4-[4-(4-aminopiperidine-1-carbonyl)piperazine-1-carbonyl]-3-chloroanilino]imidazo[1,2-a]pyrazin-3-yl]-3-(trifluoromethyl)pyrazol-1-yl]acetonitrile;
2-[4-[8-[4-[4-[(3R)-3-aminopyrrolidine-1-carbonyl]piperazine-1-carbonyl]-3-chloroanilino]imidazo[1,2-a]pyrazin-3-yl]-3-(trifluoromethyl)pyrazol-1-yl]acetonitrile;
2-[4-[8-[4-[4-[(3S)-3-aminopyrrolidine-1-carbonyl]piperazine-1-carbonyl]-3-chloroanilino]imidazo[1,2-a]pyrazin-3-yl]-3-(trifluoromethyl)pyrazol-1-yl]acetonitrile;
[2-chloro-4-[[3-[1-[(2-methylpyrazol-3-yl)methyl]-3-(trifluoromethyl)pyrazol-4-yl]imidazo[1,2-a]pyrazin-8-yl]amino]phenyl]-piperazin-1-ylmethanone;
[2-chloro-4-[[3-[1-(pyrimidin-5-ylmethyl)-3-(trifluoromethyl)pyrazol-4-yl]imidazo[1,2-a]pyrazin-8-yl]amino]phenyl]-piperazin-1-ylmethanone;
[2-chloro-4-[[3-[1-[(1-methylpyrazol-3-yl)methyl]-3-(trifluoromethyl)pyrazol-4-yl]imidazo[1,2-a]pyrazin-8-yl]amino]phenyl]-piperazin-1-ylmethanone;
2-ethyl-N-[3-(prop-2-ynylamino)propyl]-4-[[3-[3-(trifluoromethyl)-1H-pyrazol-4-yl]imidazo[1,2-a]pyrazin-8-yl]amino]benzamide;
[2-chloro-4-[[3-[1-[(1,5-dimethylpyrazol-3-yl)methyl]-3-(trifluoromethyl)pyrazol-4-yl]imidazo[1,2-a]pyrazin-8-yl]amino]phenyl]-piperazin-1-ylmethanone;
2-[4-[8-[3-chloro-4-[4-[(2R)-pyrrolidine-2-carbonyl]piperazine-1-carbonyl]anilino]imidazo[1,2-a]pyrazin-3-yl]-3-(trifluoromethyl)pyrazol-1-yl]acetonitrile;
2-[4-[8-[4-[4-[(2S)-2-aminopropanoyl]piperazine-1-carbonyl]-3-chloroanilino]imidazo[1,2-a]pyrazin-3-yl]-3-(trifluoromethyl)pyrazol-1-yl]acetonitrile;
2-[4-[8-[4-[4-[(2R)-2-aminopropanoyl]piperazine-1-carbonyl]-3-chloroanilino]imidazo[1,2-a]pyrazin-3-yl]-3-(trifluoromethyl)pyrazol-1-yl]acetonitrile;
2-[4-[8-[4-[4-(3-aminocyclobutanecarbonyl)piperazine-1-carbonyl]-3-chloroanilino]imidazo[1,2-a]pyrazin-3-yl]-3-(trifluoromethyl)pyrazol-1-yl]acetonitrile;
2-[4-[8-[3-chloro-4-[4-[(3S)-pyrrolidine-3-carbonyl]piperazine-1-carbonyl]anilino]imidazo[1,2-a]pyrazin-3-yl]-3-(trifluoromethyl)pyrazol-1-yl]acetonitrile;
2-[4-[8-[3-chloro-4-[4-[(3R)-pyrrolidine-3-carbonyl]piperazine-1-carbonyl]anilino]imidazo[1,2-a]pyrazin-3-yl]-3-(trifluoromethyl)pyrazol-1-yl]acetonitrile;
2-[4-[8-[4-[4-[(1S,3R)-3-aminocyclopentanecarbonyl]piperazine-1-carbonyl]-3-chloroanilino]imidazo[1,2-a]pyrazin-3-yl]-3-(trifluoromethyl)pyrazol-1-yl]acetonitrile;
N-[3-(2-aminoethylcarbamoylamino)propyl]-2-ethyl-4-[[3-[3-(trifluoromethyl)-1H-pyrazol-4-yl]imidazo[1,2-a]pyrazin-8-yl]amino]benzamide;
2-[4-[8-[4-[4-[(1R,3S)-3-aminocyclopentanecarbonyl]piperazine-1-carbonyl]-3-chloroanilino]imidazo[1,2-a]pyrazin-3-yl]-3-(trifluoromethyl)pyrazol-1-yl]acetonitrile;
N-[3-[[(2S)-2,6-diaminohexanoyl]amino]propyl]-2-ethyl-4-[[3-[3-(trifluoromethyl)-1H-pyrazol-4-yl]imidazo[1,2-a]pyrazin-8-yl]amino]benzamide;
2-ethyl-N-[3-(prop-2-enylamino)propyl]-4-[[3-[3-(trifluoromethyl)-1H-pyrazol-4-yl]imidazo[1,2-a]pyrazin-8-yl]amino]benzamide;
[2-chloro-4-[[3-[3-(trifluoromethyl)-1H-pyrazol-4-yl]imidazo[1,2-a]pyrazin-8-yl]amino]phenyl]-[4-[(3R)-pyrrolidine-3-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(2R)-pyrrolidine-2-carbonyl]piperazin-1-yl]methanone;
N-(4-aminocyclohexyl)-2-chloro-4-[[3-[1-(cyanomethyl)-3-(trifluoromethyl)pyrazol-4-yl]imidazo[1,2-a]pyrazin-8-yl]amino]benzamide;
[4-[(2S,4R)-4-aminopyrrolidine-2-carbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
N-(2-aminoethoxy)-2-chloro-4-[[3-[1-(cyanomethyl)-3-(trifluoromethyl)pyrazol-4-yl]imidazo[1,2-a]pyrazin-8-yl]amino]benzamide;
2-[4-[8-[4-(4-aminopiperidine-1-carbonyl)-3-chloroanilino]imidazo[1,2-a]pyrazin-3-yl]-3-(trifluoromethyl)pyrazol-1-yl]acetonitrile;
N-[2-[(2S)-2-aminopropoxy]ethyl]-2-ethyl-4-[[3-[3-(trifluoromethyl)-1H-pyrazol-4-yl]imidazo[1,2-a]pyrazin-8-yl]amino]benzamide;
N-[2-[(2R)-2-aminopropoxy]ethyl]-2-ethyl-4-[[3-[3-(trifluoromethyl)-1H-pyrazol-4-yl]imidazo[1,2-a]pyrazin-8-yl]amino]benzamide;
4-[[3-[1-(cyanomethyl)-3-(trifluoromethyl)pyrazol-4-yl]imidazo[1,2-a]pyrazin-8-yl]amino]-N-(3-ethoxypropyl)-2-ethylbenzamide;
(2S)-2-amino-1-[4-[2-chloro-4-[[3-[3-(trifluoromethyl)-1H-pyrazol-4-yl]imidazo[1,2-a]pyrazin-8-yl]amino]benzoyl]piperazin-1-yl]propan-1-one;
(2R)-2-amino-1-[4-[2-chloro-4-[[3-[3-(trifluoromethyl)-1H-pyrazol-4-yl]imidazo[1,2-a]pyrazin-8-yl]amino]benzoyl]piperazin-1-yl]propan-1-one;
[2-chloro-4-[[3-[3-(trifluoromethyl)-1H-pyrazol-4-yl]imidazo[1,2-a]pyrazin-8-yl]amino]phenyl]-[4-[(3S)-pyrrolidine-3-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(2S)-pyrrolidine-2-carbonyl]piperazin-1-yl]methanone;
2-[4-[8-[3-chloro-4-[4-[(2S)-pyrrolidine-2-carbonyl]piperazine-1-carbonyl]anilino]imidazo[1,2-a]pyrazin-3-yl]-3-(trifluoromethyl)pyrazol-1-yl]acetonitrile;
N-[3-[[(2S)-2-amino-3-carbamimidamidopropanoyl]amino]propyl]-2-chloro-4-[[3-[3-(trifluoromethyl)-1H-pyrazol-4-yl]imidazo[1,2-a]pyrazin-8-yl]amino]benzamide;
N-[3-(3-aminopropanoylamino)propyl]-2-ethyl-4-[[3-[3-(trifluoromethyl)-1H-pyrazol-4-yl]imidazo[1,2-a]pyrazin-8-yl]amino]benzamide;
N-[2-[[(2S)-2-amino-3-carbamimidamidopropanoyl]amino]ethyl]-2-ethyl-4-[[3-[3-(trifluoromethyl)-1H-pyrazol-4-yl]imidazo[1,2-a]pyrazin-8-yl]amino]benzamide;
N-(azetidin-3-yl)-4-[2-chloro-4-[[3-[1-(cyanomethyl)-3-(trifluoromethyl)pyrazol-4-yl]imidazo[1,2-a]pyrazin-8-yl]amino]benzoyl]piperazine-1-carboxamide;
N-[3-(4-aminobutanoylamino)propyl]-2-ethyl-4-[[3-[3-(trifluoromethyl)-1H-pyrazol-4-yl]imidazo[1,2-a]pyrazin-8-yl]amino]benzamide;
N-[2-[[(2S)-2-amino-3-carbamimidamidopropanoyl]amino]ethyl]-2-chloro-4-[[3-[3-(trifluoromethyl)-1H-pyrazol-4-yl]imidazo[1,2-a]pyrazin-8-yl]amino]benzamide;
N-[2-(4-aminobutanoylamino)ethyl]-2-ethyl-4-[[3-[3-(trifluoromethyl)-1H-pyrazol-4-yl]imidazo[1,2-a]pyrazin-8-yl]amino]benzamide;
N-[2-(2-aminoethylcarbamoylamino)ethyl]-2-ethyl-4-[[3-[3-(trifluoromethyl)-1H-pyrazol-4-yl]imidazo[1,2-a]pyrazin-8-yl]amino]benzamide;
2-[4-[8-[3-ethyl-4-[4-hydroxy-4-(methylaminomethyl)piperidine-1-carbonyl]anilino]imidazo[1,2-a]pyrazin-3-yl]-3-(trifluoromethyl)pyrazol-1-yl]acetonitrile;
N-[2-(3-aminopropanoylamino)ethyl]-2-ethyl-4-[[3-[3-(trifluoromethyl)-1H-pyrazol-4-yl]imidazo[1,2-a]pyrazin-8-yl]amino]benzamide;
N-[2-[[(2S)-2,6-diaminohexanoyl]amino]ethyl]-2-ethyl-4-[[3-[3-(trifluoromethyl)-1H-pyrazol-4-yl]imidazo[1,2-a]pyrazin-8-yl]amino]benzamide;
N-[2-(2-aminoethylcarbamoylamino)ethyl]-2-chloro-4-[[3-[3-(trifluoromethyl)-1H-pyrazol-4-yl]imidazo[1,2-a]pyrazin-8-yl]amino]benzamide;
N-(3-ethoxypropyl)-2-ethyl-4-[[3-[3-(trifluoromethyl)-1H-pyrazol-4-yl]imidazo[1,2-a]pyrazin-8-yl]amino]benzamide;
2-chloro-N-[2-[[(2S)-2,6-diaminohexanoyl]amino]ethyl]-4-[[3-[3-(trifluoromethyl)-1H-pyrazol-4-yl]imidazo[1,2-a]pyrazin-8-yl]amino]benzamide;
[4-[(1R,2S)-2-aminocyclopropanecarbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
2-[4-[8-[3-chloro-4-(piperazine-1-carbonyl)anilino]imidazo[1,2-a]pyrazin-3-yl]-3-(trifluoromethyl)pyrazol-1-yl]acetonitrile;
N-(6-aminohexyl)-4-[[3-[1-(cyanomethyl)-3-(trifluoromethyl)pyrazol-4-yl]imidazo[1,2-a]pyrazin-8-yl]amino]-2-ethylbenzamide;
N-(2-aminoethoxy)-4-[[3-[1-(cyanomethyl)-3-(trifluoromethyl)pyrazol-4-yl]imidazo[1,2-a]pyrazin-8-yl]amino]-2-ethylbenzamide;
N-(2-aminoethyl)-2-ethyl-4-[[3-[3-(trifluoromethyl)-1H-pyrazol-4-yl]imidazo[1,2-a]pyrazin-8-yl]amino]benzamide;
2-chloro-4-[[3-[1-(cyanomethyl)-3-(trifluoromethyl)pyrazol-4-yl]imidazo[1,2-a]pyrazin-8-yl]amino]-N-ethylbenzamide;
2-chloro-N-(cyanomethyl)-4-[[3-[1-(cyanomethyl)-3-(trifluoromethyl)pyrazol-4-yl]imidazo[1,2-a]pyrazin-8-yl]amino]benzamide;
N-[2-(2-aminoethoxy)ethyl]-4-[[3-[1-but-3-yn-2-yl-3-(trifluoromethyl)pyrazol-4-yl]imidazo[1,2-a]pyrazin-8-yl]amino]-2-ethylbenzamide;
N-[(1S,3R)-3-aminocyclopentyl]-4-[2-chloro-4-[[3-[3-(trifluoromethyl)-1H-pyrazol-4-yl]imidazo[1,2-a]pyrazin-8-yl]amino]benzoyl]piperazine-1-carboxamide;
2-chloro-4-[[3-[1-(cyanomethyl)-3-(trifluoromethyl)pyrazol-4-yl]imidazo[1,2-a]pyrazin-8-yl]amino]-N-(2-hydroxyethyl)benzamide;
N-[2-(2-aminoethoxy)ethyl]-2-ethyl-4-[[3-[1-[(5-methyl-1,2-oxazol-3-yl)methyl]-3-(trifluoromethyl)pyrazol-4-yl]imidazo[1,2-a]pyrazin-8-yl]amino]benzamide;
2-[4-[8-[3-chloro-4-(3-hydroxyazetidine-1-carbonyl)anilino]imidazo[1,2-a]pyrazin-3-yl]-3-(trifluoromethyl)pyrazol-1-yl]acetonitrile;
2-chloro-4-[[3-[1-(cyanomethyl)-3-(trifluoromethyl)pyrazol-4-yl]imidazo[1,2-a]pyrazin-8-yl]amino]-N-[(2R)-2-hydroxypropyl]benzamide;
N-[(1R,3S)-3-aminocyclopentyl]-4-[2-chloro-4-[[3-[3-(trifluoromethyl)-1H-pyrazol-4-yl]imidazo[1,2-a]pyrazin-8-yl]amino]benzoyl]piperazine-1-carboxamide; 2-chloro-4-[[3-[1-(cyanomethyl)-3-(trifluoromethyl)pyrazol-4-yl]imidazo[1,2-a]pyrazin-8-yl]amino]-N-(2-methoxyethyl)benzamide; 2-chloro-4-[[3-[1-(cyanomethyl)-3-(trifluoromethyl)pyrazol-4-yl]imidazo[1,2-a]pyrazin-8-yl]amino]-N-[(2S)-2-hydroxypropyl]benzamide;
N-[2-(2-aminoethoxy)ethyl]-2-ethyl-4-[[3-[1-(oxetan-3-yl)-3-(trifluoromethyl)pyrazol-4-yl]imidazo[1,2-a]pyrazin-8-yl]amino]benzamide;
2-[4-[8-[3-chloro-4-(3-hydroxy-3-methylazetidine-1-carbonyl)anilino]imidazo[1,2-a]pyrazin-3-yl]-3-(trifluoromethyl)pyrazol-1-yl]acetonitrile;
2-[4-[8-[3-chloro-4-[(3S)-3-hydroxypyrrolidine-1-carbonyl]anilino]imidazo[1,2-a]pyrazin-3-yl]-3-(trifluoromethyl)pyrazol-1-yl]acetonitrile;
2-[4-[8-[3-chloro-4-[(3R)-3-hydroxypyrrolidine-1-carbonyl]anilino]imidazo[1,2-a]pyrazin-3-yl]-3-(trifluoromethyl)pyrazol-1-yl]acetonitrile;
[4-[(2R,4S)-4-aminopyrrolidine-2-carbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
N-(azetidin-3-yl)-4-[2-chloro-4-[[3-[1-(2-cyanoethyl)-3-(trifluoromethyl)pyrazol-4-yl]imidazo[1,2-a]pyrazin-8-yl]amino]benzoyl]piperazine-1-carboxamide;
[2-chloro-4-[[3-[3-(trifluoromethyl)-1H-pyrazol-4-yl]imidazo[1,2-a]pyrazin-8-yl]amino]phenyl]-[4-[(3S)-3-hydroxypiperidine-3-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(3R)-3-hydroxypiperidine-3-carbonyl]piperazin-1-yl]methanone;
N-[2-(2-aminoethoxy)ethyl]-2-chloro-4-[[3-[1-prop-2-ynyl-3-(trifluoromethyl)pyrazol-4-yl]imidazo[1,2-a]pyrazin-8-yl]amino]benzamide;
N-[2-(2-aminoethoxy)ethyl]-2-chloro-4-[[3-[1-(2-fluoroethyl)-3-(trifluoromethyl)pyrazol-4-yl]imidazo[1,2-a]pyrazin-8-yl]amino]benzamide;
N-(cyanomethyl)-4-[[3-[1-(cyanomethyl)-3-(trifluoromethyl)pyrazol-4-yl]imidazo[1,2-a]pyrazin-8-yl]amino]-2-ethylbenzamide;
N-[2-(2-aminoethoxy)ethyl]-2-chloro-4-[[3-[1-(2,2-difluoroethyl)-3-(trifluoromethyl)pyrazol-4-yl]imidazo[1,2-a]pyrazin-8-yl]amino]benzamide;
N-[2-(2-aminoethoxy)ethyl]-4-[[3-[1-(2-fluoroethyl)-3-(trifluoromethyl)pyrazol-4-yl]imidazo[1,2-a]pyrazin-8-yl]amino]-2-methylbenzamide;
N-[2-(2-aminoethoxy)ethyl]-4-[[3-[1-(2,2-difluoroethyl)-3-(trifluoromethyl)pyrazol-4-yl]imidazo[1,2-a]pyrazin-8-yl]amino]-2-methylbenzamide;
N-(4-aminocyclohexyl)-4-[[3-[1-(cyanomethyl)-3-(trifluoromethyl)pyrazol-4-yl]imidazo[1,2-a]pyrazin-8-yl]amino]-2-ethylbenzamide;
3-[4-[8-[4-[4-(3-aminopropanoyl)piperazine-1-carbonyl]-3-chloroanilino]imidazo[1,2-a]pyrazin-3-yl]-3-(trifluoromethyl)pyrazol-1-yl]propanenitrile;
N-[2-(2-aminoethoxy)ethyl]-2-chloro-4-[[3-[1-(cyanomethyl)-3-(trifluoromethyl)pyrazol-4-yl]imidazo[1,2-a]pyrazin-8-yl]amino]benzamide;
N-(2-aminoethyl)-4-[2-chloro-4-[[3-[1-(2-cyanoethyl)-3-(trifluoromethyl)pyrazol-4-yl]imidazo[1,2-a]pyrazin-8-yl]amino]benzoyl]piperazine-1-carboxamide;
4-[2-chloro-4-[[3-[1-(2-cyanoethyl)-3-(trifluoromethyl)pyrazol-4-yl]imidazo[1,2-a]pyrazin-8-yl]amino]benzoyl]-N-[(3S)-pyrrolidin-3-yl]piperazine-1-carboxamide;
2-[4-[8-[3-ethyl-4-(piperazine-1-carbonyl)anilino]imidazo[1,2-a]pyrazin-3-yl]-3-(trifluoromethyl)pyrazol-1-yl]acetonitrile;
4-[2-chloro-4-[[3-[1-(2-cyanoethyl)-3-(trifluoromethyl)pyrazol-4-yl]imidazo[1,2-a]pyrazin-8-yl]amino]benzoyl]-N-[(3R)-pyrrolidin-3-yl]piperazine-1-carboxamide;
N-[2-(2-aminoethoxy)ethyl]-4-[[3-[1-(cyanomethyl)-3-(trifluoromethyl)pyrazol-4-yl]imidazo[1,2-a]pyrazin-8-yl]amino]-2-methylbenzamide;
N-[2-(1-aminocyclopropyl)ethyl]-2-ethyl-4-[[3-[3-(trifluoromethyl)-1H-pyrazol-4-yl]imidazo[1,2-a]pyrazin-8-yl]amino]benzamide;
N-(2-aminoethyl)-4-[2-chloro-4-[[3-[1-(cyanomethyl)-3-(trifluoromethyl)pyrazol-4-yl]imidazo[1,2-a]pyrazin-8-yl]amino]benzoyl]piperazine-1-carboxamide;
N-(3-aminocyclobutyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
[4-(3-aminoazetidine-1-carbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone;
N-[2-(2-aminoethoxy)ethyl]-2-ethyl-4-[[3-[1-(3-hydroxy-2-methylpropyl)-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(2-hydroxybutyl)-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(2-pyrrolidin-1-ylethyl)-3-(trifluoromethyl)pyrazol-4-yl]imidazo[1,2-a]pyrazin-8-yl]amino]benzamide;
N-[3-[4-(3-aminopropylamino)butylamino]propyl]-2-ethyl-4-[[3-[3-(trifluoromethyl)-1H-pyrazol-4-yl]imidazo[1,2-a]pyrazin-8-yl]amino]benzamide;
2-[4-[8-[4-[2-(2-aminoethoxy)ethylcarbamoyl]-3-ethylanilino]imidazo[1,2-a]pyrazin-3-yl]-3-(trifluoromethyl)pyrazol-1-yl]acetic acid;
N-[2-(2-aminoethoxy)ethyl]-2-ethyl-4-[[3-[1-prop-2-enyl-3-(trifluoromethyl)pyrazol-4-yl]imidazo[1,2-a]pyrazin-8-yl]amino]benzamide;
N-[2-(2-aminoethoxy)ethyl]-4-[[3-[1-cyclobutyl-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(2-chloroprop-2-enyl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(3-chlorobut-3-en-2-yl)-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(2,2,2-trifluoroethyl)-3-(trifluoromethyl)pyrazol-4-yl]imidazo[1,2-a]pyrazin-8-yl]amino]benzamide;
N-[2-(2-aminoethoxy)ethyl]-4-[[3-[1-[(2,2-difluorocyclopropyl)methyl]-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(1-chloro-3-hydroxypropan-2-yl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-[2-(chloromethyl)-3-hydroxy-2-methylpropyl]-3-(trifluoromethyl)pyrazol-4-yl]imidazo[1,2-a]pyrazin-8-yl]amino]-2-ethylbenzamide;
N,2-diethyl-4-[[3-[3-(trifluoromethyl)-1H-pyrazol-4-yl]imidazo[1,2-a]pyrazin-8-yl]amino]benzamide;
2-chloro-N-methyl-4-[[3-[1-(pyrimidin-4-ylmethyl)-3-(trifluoromethyl)pyrazol-4-yl]imidazo[1,2-a]pyrazin-8-yl]amino]benzamide;
2-ethyl-N-methyl-4-[[3-[1-(pyrimidin-4-ylmethyl)-3-(trifluoromethyl)pyrazol-4-yl]imidazo[1,2-a]pyrazin-8-yl]amino]benzamide;
4-[[3-[1-(2-aminoethyl)-3-(trifluoromethyl)pyrazol-4-yl]imidazo[1,2-a]pyrazin-8-yl]amino]-2-ethyl-N-methylbenzamide;
2-chloro-N-methyl-4-[[3-[1-(1H-pyrazol-3-ylmethyl)-3-(trifluoromethyl)pyrazol-4-yl]imidazo[1,2-a]pyrazin-8-yl]amino]benzamide;
[4-(4-aminopiperidine-1-carbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone;
2-[4-[8-[4-[4-(3-aminopropanoyl)piperazine-1-carbonyl]-3-chloroanilino]imidazo[1,2-a]pyrazin-3-yl]-3-(trifluoromethyl)pyrazol-1-yl]acetonitrile;
N-(4-aminocyclohexyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
4-[2-chloro-4-[[3-[1-(cyanomethyl)-3-(trifluoromethyl)pyrazol-4-yl]imidazo[1,2-a]pyrazin-8-yl]amino]benzoyl]-N-[(3R)-pyrrolidin-3-yl]piperazine-1-carboxamide;
N-(3-aminocyclobutyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
N-(2-aminoethyl)-4-[2-chloro-4-[[3-[1-(2-methoxyethyl)-3-(trifluoromethyl)pyrazol-4-yl]imidazo[1,2-a]pyrazin-8-yl]amino]benzoyl]piperazine-1-carboxamide;
N-(4-aminocyclohexyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
N-[(1S,3S)-3-aminocyclopentyl]-4-[2-chloro-4-[[3-[3-(trifluoromethyl)-1H-pyrazol-4-yl]imidazo[1,2-a]pyrazin-8-yl]amino]benzoyl]piperazine-1-carboxamide;
4-[2-chloro-4-[[3-[1-(2-methoxyethyl)-3-(trifluoromethyl)pyrazol-4-yl]imidazo[1,2-a]pyrazin-8-yl]amino]benzoyl]-N-[(3S)-pyrrolidin-3-yl]piperazine-1-carboxamide;
3-amino-1-[4-[2-chloro-4-[[3-[1-(2-methoxyethyl)-3-(trifluoromethyl)pyrazol-4-yl]imidazo[1,2-a]pyrazin-8-yl]amino]benzoyl]piperazin-1-yl]propan-1-one;
[4-[(2S,4S)-4-aminopyrrolidine-2-carbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
4-[2-chloro-4-[[3-[1-(2-methoxyethyl)-3-(trifluoromethyl)pyrazol-4-yl]imidazo[1,2-a]pyrazin-8-yl]amino]benzoyl]-N-[(3R)-pyrrolidin-3-yl]piperazine-1-carboxamide;
4-[2-chloro-4-[[3-[1-(cyanomethyl)-3-(trifluoromethyl)pyrazol-4-yl]imidazo[1,2-a]pyrazin-8-yl]amino]benzoyl]-N-[(3S)-pyrrolidin-3-yl]piperazine-1-carboxamide;
2-ethyl-N-methyl-4-[[3-[1-[2-(methylamino)ethyl]-3-(trifluoromethyl)pyrazol-4-yl]imidazo[1,2-a]pyrazin-8-yl]amino]benzamide;
N-[2-(3-aminopropanoylamino)ethyl]-2-chloro-4-[[3-[3-(trifluoromethyl)-1H-pyrazol-4-yl]imidazo[1,2-a]pyrazin-8-yl]amino]benzamide;
N-[2-(4-aminobutanoylamino)ethyl]-2-chloro-4-[[3-[3-(trifluoromethyl)-1H-pyrazol-4-yl]imidazo[1,2-a]pyrazin-8-yl]amino]benzamide;
N-[2-(2-aminoethoxy)ethyl]-2-ethyl-4-[[3-[1-(2-fluoroethyl)-3-(trifluoromethyl)pyrazol-4-yl]imidazo[1,2-a]pyrazin-8-yl]amino]benzamide;
N-[2-(2-aminoethoxy)ethyl]-4-[[3-[1-(2,2-difluoroethyl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(cyclobutylmethyl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-[(3,3-difluorocyclobutyl)methyl]-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(2-hydroxyethyl)-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(3-hydroxy-2,2-dimethylpropyl)-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(oxolan-3-ylmethyl)-3-(trifluoromethyl)pyrazol-4-yl]imidazo[1,2-a]pyrazin-8-yl]amino]benzamide;
2-chloro-N-[(3R)-1-[(2S,4R)-4-hydroxypyrrolidine-2-carbonyl]pyrrolidin-3-yl]-4-[[3-[3-(trifluoromethyl)-1H-pyrazol-4-yl]imidazo[1,2-a]pyrazin-8-yl]amino]benzamide;
N-[2-(2-aminoethoxy)ethyl]-4-[[3-[1-[2-(dimethylamino)ethyl]-3-(trifluoromethyl)pyrazol-4-yl]imidazo[1,2-a]pyrazin-8-yl]amino]-2-ethylbenzamide;
ethyl 2-[4-[8-[4-[2-(2-aminoethoxy)ethylcarbamoyl]-3-ethylanilino]imidazo[1,2-a]pyrazin-3-yl]-3-(trifluoromethyl)pyrazol-1-yl]acetate;
N-[2-(2-aminoethoxy)ethyl]-2-ethyl-4-[[3-[1-(3-hydroxy-3-methylbutyl)-3-(trifluoromethyl)pyrazol-4-yl]imidazo[1,2-a]pyrazin-8-yl]amino]benzamide;
N-[2-(2-aminoethoxy)ethyl]-2-ethyl-4-[[3-[1-(3-hydroxypropyl)-3-(trifluoromethyl)pyrazol-4-yl]imidazo[1,2-a]pyrazin-8-yl]amino]benzamide;
2-chloro-N-[(3S)-1-[(2S,4R)-4-hydroxypyrrolidine-2-carbonyl]pyrrolidin-3-yl]-4-[[3-[3-(trifluoromethyl)-1H-pyrazol-4-yl]imidazo[1,2-a]pyrazin-8-yl]amino]benzamide;
N-[2-(2-aminoethoxy)ethyl]-4-[[3-[1-(2,3-dihydroxypropyl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(2,4-dihydroxybutyl)-3-(trifluoromethyl)pyrazol-4-yl]imidazo[1,2-a]pyrazin-8-yl]amino]-2-ethylbenzamide;
N-[2-(2-aminoethoxy)ethyl]-4-[[3-[1-(2-amino-2-oxoethyl)-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(2-morpholin-4-ylethyl)-3-(trifluoromethyl)pyrazol-4-yl]imidazo[1,2-a]pyrazin-8-yl]amino]benzamide;
(4-aminopiperidin-1-yl)-[2-chloro-4-[[3-[3-(trifluoromethyl)-1H-pyrazol-4-yl]imidazo[1,2-a]pyrazin-8-yl]amino]phenyl]methanone;
N-(5-aminopentyl)-2-ethyl-4-[[3-[3-(trifluoromethyl)-1H-pyrazol-4-yl]imidazo[1,2-a]pyrazin-8-yl]amino]benzamide;
[(3S)-3-aminopyrrolidin-1-yl]-[2-chloro-4-[[3-[3-(trifluoromethyl)-1H-pyrazol-4-yl]imidazo[1,2-a]pyrazin-8-yl]amino]phenyl]methanone;
[2-chloro-4-[[3-[3-(trifluoromethyl)-1H-pyrazol-4-yl]imidazo[1,2-a]pyrazin-8-yl]amino]phenyl]-[(3S)-3-methylpiperazin-1-yl]methanone;
N-(2-aminoethyl)-2-chloro-N-methyl-4-[[3-[3-(trifluoromethyl)-1H-pyrazol-4-yl]imidazo[1,2-a]pyrazin-8-yl]amino]benzamide;
[2-chloro-4-[[3-[3-(trifluoromethyl)-1H-pyrazol-4-yl]imidazo[1,2-a]pyrazin-8-yl]amino]phenyl]-[(3R)-3-methylpiperazin-1-yl]methanone;
[(3R)-3-aminopyrrolidin-1-yl]-[2-chloro-4-[[3-[3-(trifluoromethyl)-1H-pyrazol-4-yl]imidazo[1,2-a]pyrazin-8-yl]amino]phenyl]methanone;
[2-chloro-4-[[3-[3-(trifluoromethyl)-1H-pyrazol-4-yl]imidazo[1,2-a]pyrazin-8-yl]amino]phenyl]-[(2S)-2-methylpiperazin-1-yl]methanone;
[4-[(3R)-3-aminopyrrolidine-1-carbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
[4-(3-aminocyclobutanecarbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone;
[4-[(3S)-3-aminopyrrolidine-1-carbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
N-(5-aminopentyl)-4-[[3-[1-(cyanomethyl)-3-(trifluoromethyl)pyrazol-4-yl]imidazo[1,2-a]pyrazin-8-yl]amino]-2-ethylbenzamide;
[4-(3-aminocyclobutanecarbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone;
(2S)-2-amino-1-[4-[2-methyl-4-[[3-[3-(trifluoromethyl)-1H-pyrazol-4-yl]imidazo[1,2-a]pyrazin-8-yl]amino]benzoyl]piperazin-1-yl]propan-1-one;
[2-methyl-4-[[3-[3-(trifluoromethyl)-1H-pyrazol-4-yl]imidazo[1,2-a]pyrazin-8-yl]amino]phenyl]-[4-[(3R)-pyrrolidine-3-carbonyl]piperazin-1-yl]methanone;
[2-methyl-4-[[3-[3-(trifluoromethyl)-1H-pyrazol-4-yl]imidazo[1,2-a]pyrazin-8-yl]amino]phenyl]-[4-[(3S)-pyrrolidine-3-carbonyl]piperazin-1-yl]methanone;
(2R)-2-amino-1-[4-[2-methyl-4-[[3-[3-(trifluoromethyl)-1H-pyrazol-4-yl]imidazo[1,2-a]pyrazin-8-yl]amino]benzoyl]piperazin-1-yl]propan-1-one;
2-ethyl-N-methyl-4-[[3-[1-prop-2-ynyl-3-(trifluoromethyl)pyrazol-4-yl]imidazo[1,2-a]pyrazin-8-yl]amino]benzamide;
N-(6-aminohexyl)-2-ethyl-4-[[3-[3-(trifluoromethyl)-1H-pyrazol-4-yl]imidazo[1,2-a]pyrazin-8-yl]amino]benzamide;
4-[[3-[1-(2-cyanoethyl)-3-(trifluoromethyl)pyrazol-4-yl]imidazo[1,2-a]pyrazin-8-yl]amino]-2-ethyl-N-methylbenzamide;
N-[3-(3-aminopropanoylamino)propyl]-2-chloro-4-[[3-[3-(trifluoromethyl)-1H-pyrazol-4-yl]imidazo[1,2-a]pyrazin-8-yl]amino]benzamide;
4-[[3-[1-(2-amino-2-oxoethyl)-3-(trifluoromethyl)pyrazol-4-yl]imidazo[1,2-a]pyrazin-8-yl]amino]-2-ethyl-N-methylbenzamide;
[4-[(1R,3S)-3-aminocyclopentanecarbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
4-[[3-[1-(cyanomethyl)-3-(trifluoromethyl)pyrazol-4-yl]imidazo[1,2-a]pyrazin-8-yl]amino]-2-ethyl-N-methylbenzamide;
[4-[(1S,3R)-3-aminocyclopentanecarbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
4-[[3-[1-(3-cyanopropyl)-3-(trifluoromethyl)pyrazol-4-yl]imidazo[1,2-a]pyrazin-8-yl]amino]-2-ethyl-N-methylbenzamide;
N-[3-(4-aminobutanoylamino)propyl]-2-chloro-4-[[3-[3-(trifluoromethyl)-1H-pyrazol-4-yl]imidazo[1,2-a]pyrazin-8-yl]amino]benzamide;
2-chloro-N-[3-[[(2S)-2,6-diaminohexanoyl]amino]propyl]-4-[[3-[3-(trifluoromethyl)-1H-pyrazol-4-yl]imidazo[1,2-a]pyrazin-8-yl]amino]benzamide;
N-[2-(2-aminoethoxy)ethyl]-4-[[3-[3-(difluoromethyl)-1-propylpyrazol-4-yl]imidazo[1,2-a]pyrazin-8-yl]amino]-2-ethylbenzamide;
N-[2-(2-aminoethoxy)ethyl]-4-[[3-[3-(difluoromethyl)-1-methylpyrazol-4-yl]imidazo[1,2-a]pyrazin-8-yl]amino]-2-ethylbenzamide;
4-[[3-[1-(azetidin-3-yl)-3-(trifluoromethyl)pyrazol-4-yl]imidazo[1,2-a]pyrazin-8-yl]amino]-2-chloro-N-methylbenzamide;
N-[(2S)-1-aminopropan-2-yl]-2-ethyl-4-[[3-[3-(trifluoromethyl)-1H-pyrazol-4-yl]imidazo[1,2-a]pyrazin-8-yl]amino]benzamide;
2-ethyl-N-methyl-4-[[3-[3-(trifluoromethyl)-1H-pyrazol-4-yl]imidazo[1,2-a]pyrazin-8-yl]amino]benzamide;
2-chloro-N-methyl-4-[[3-[1-prop-2-ynyl-3-(trifluoromethyl)pyrazol-4-yl]imidazo[1,2-a]pyrazin-8-yl]amino]benzamide;
[2-chloro-4-[[3-[3-(trifluoromethyl)-1H-pyrazol-4-yl]imidazo[1,2-a]pyrazin-8-yl]amino]phenyl]-[4-(3-hydroxypiperidine-3-carbonyl)piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[rac-(3R,5R)-5-hydroxypiperidine-3-carbonyl]piperazin-1-yl]methanone;
[2-ethyl-4-[[3-[3-(trifluoromethyl)-1H-pyrazol-4-yl]imidazo[1,2-a]pyrazin-8-yl]amino]phenyl]-[4-(piperidine-4-carbonyl)piperazin-1-yl]methanone;
[2-ethyl-4-[[3-[3-(trifluoromethyl)-1H-pyrazol-4-yl]imidazo[1,2-a]pyrazin-8-yl]amino]phenyl]-[4-(4-hydroxypiperidine-4-carbonyl)piperazin-1-yl]methanone;
2-chloro-4-[[3-[1-(cyanomethyl)-3-(trifluoromethyl)pyrazol-4-yl]imidazo[1,2-a]pyrazin-8-yl]amino]-N-methylbenzamide;
4-[[3-[1-(2-amino-2-oxoethyl)-3-(trifluoromethyl)pyrazol-4-yl]imidazo[1,2-a]pyrazin-8-yl]amino]-2-chloro-N-methylbenzamide;
2-chloro-4-[[3-[1-(2-cyanoethyl)-3-(trifluoromethyl)pyrazol-4-yl]imidazo[1,2-a]pyrazin-8-yl]amino]-N-methylbenzamide;
2-chloro-4-[[3-[1-(3-cyanopropyl)-3-(trifluoromethyl)pyrazol-4-yl]imidazo[1,2-a]pyrazin-8-yl]amino]-N-methylbenzamide;
N-[2-(2-aminoethoxy)ethyl]-2-methyl-4-[[3-[3-(trifluoromethyl)-1H-pyrazol-4-yl]imidazo[1,2-a]pyrazin-8-yl]amino]benzamide;
N-[2-(2-aminoethoxy)ethyl]-2-chloro-4-[[3-[3-(trifluoromethyl)-1H-pyrazol-4-yl]imidazo[1,2-a]pyrazin-8-yl]amino]benzamide;
N-(4-aminobutyl)-2-chloro-4-[[3-[3-(trifluoromethyl)-1H-pyrazol-4-yl]imidazo[1,2-a]pyrazin-8-yl]amino]benzamide;
N-(2-aminoethyl)-2-chloro-4-[[3-[3-(trifluoromethyl)-1H-pyrazol-4-yl]imidazo[1,2-a]pyrazin-8-yl]amino]benzamide;
N-(3-aminopropyl)-2-chloro-4-[[3-[3-(trifluoromethyl)-1H-pyrazol-4-yl]imidazo[1,2-a]pyrazin-8-yl]amino]benzamide;
N-(3-aminopropyl)-2-ethyl-4-[[3-[3-(trifluoromethyl)-1H-pyrazol-4-yl]imidazo[1,2-a]pyrazin-8-yl]amino]benzamide;
2-[4-[8-[3-chloro-4-[4-(piperidine-4-carbonyl)piperazine-1-carbonyl]anilino]imidazo[1,2-a]pyrazin-3-yl]-3-(trifluoromethyl)pyrazol-1-yl]acetonitrile;
(2S,4R)—N-[3-[[4-[[3-[1-(cyanomethyl)-3-(trifluoromethyl)pyrazol-4-yl]imidazo[1,2-a]pyrazin-8-yl]amino]-2-methylbenzoyl]amino]propyl]-4-hydroxypyrrolidine-2-carboxamide;
(2S,4R)—N-[3-[[2-chloro-4-[[3-[3-(trifluoromethyl)-1H-pyrazol-4-yl]imidazo[1,2-a]pyrazin-8-yl]amino]benzoyl]amino]cyclobutyl]-4-hydroxypyrrolidine-2-carboxamide;
2-chloro-N-[1-[(2S,4R)-4-hydroxypyrrolidine-2-carbonyl]azetidin-3-yl]-4-[[3-[3-(trifluoromethyl)-1H-pyrazol-4-yl]imidazo[1,2-a]pyrazin-8-yl]amino]benzamide;
[4-(aminomethyl)piperidin-1-yl]-[2-ethyl-4-[[3-[3-(trifluoromethyl)-1H-pyrazol-4-yl]imidazo[1,2-a]pyrazin-8-yl]amino]phenyl]methanone;
rac-(2R,4S)—N-[3-[[2-chloro-4-[[3-[3-(trifluoromethyl)-1H-pyrazol-4-yl]imidazo[1,2-a]pyrazin-8-yl]amino]benzoyl]amino]cyclopentyl]-4-hydroxypyrrolidine-2-carboxamide;
(2S,4R)—N-[4-[[2-chloro-4-[[3-[3-(trifluoromethyl)-1H-pyrazol-4-yl]imidazo[1,2-a]pyrazin-8-yl]amino]benzoyl]amino]cyclohexyl]-4-hydroxypyrrolidine-2-carboxamide;
(2S,4R)—N-[2-[[4-[[3-[1-(cyanomethyl)-3-(trifluoromethyl)pyrazol-4-yl]imidazo[1,2-a]pyrazin-8-yl]amino]-2-methylbenzoyl]amino]ethyl]-4-hydroxypyrrolidine-2-carboxamide;
4-[4-[[3-[1-(cyanomethyl)-3-(trifluoromethyl)pyrazol-4-yl]imidazo[1,2-a]pyrazin-8-yl]amino]-2-methylbenzoyl]-N-[(3S,4S)-4-hydroxypyrrolidin-3-yl]piperazine-1-carboxamide;
2-chloro-N-[(3S)-pyrrolidin-3-yl]-4-[[3-[3-(trifluoromethyl)-1H-pyrazol-4-yl]imidazo[1,2-a]pyrazin-8-yl]amino]benzamide;
2-chloro-N-[(3R)-pyrrolidin-3-yl]-4-[[3-[3-(trifluoromethyl)-1H-pyrazol-4-yl]imidazo[1,2-a]pyrazin-8-yl]amino]benzamide;
2-[4-[8-[4-[4-[(3S,4R)-3-hydroxypiperidine-4-carbonyl]piperazine-1-carbonyl]-3-methylanilino]imidazo[1,2-a]pyrazin-3-yl]-3-(trifluoromethyl)pyrazol-1-yl]acetonitrile;
2-chloro-N-[1-[(2S,4R)-4-hydroxypyrrolidine-2-carbonyl]piperidin-4-yl]-4-[[3-[3-(trifluoromethyl)-1H-pyrazol-4-yl]imidazo[1,2-a]pyrazin-8-yl]amino]benzamide;
2-[4-[8-[4-[4-[(3R,4S)-3-hydroxypiperidine-4-carbonyl]piperazine-1-carbonyl]-3-methylanilino]imidazo[1,2-a]pyrazin-3-yl]-3-(trifluoromethyl)pyrazol-1-yl]acetonitrile;
N-(3-aminocyclobutyl)-2-chloro-4-[[3-[3-(trifluoromethyl)-1H-pyrazol-4-yl]imidazo[1,2-a]pyrazin-8-yl]amino]benzamide;
N-(4-aminocyclohexyl)-2-chloro-4-[[3-[3-(trifluoromethyl)-1H-pyrazol-4-yl]imidazo[1,2-a]pyrazin-8-yl]amino]benzamide;
2-chloro-N-[1-[rac-(2R,4S)-4-hydroxypyrrolidine-2-carbonyl]piperidin-3-yl]-4-[[3-[3-(trifluoromethyl)-1H-pyrazol-4-yl]imidazo[1,2-a]pyrazin-8-yl]amino]benzamide;
2-chloro-N-methyl-4-[[3-[3-(trifluoromethyl)-1H-pyrazol-4-yl]imidazo[1,2-a]pyrazin-8-yl]amino]benzamide;
4-[2-chloro-4-[[3-[3-(trifluoromethyl)-1H-pyrazol-4-yl]imidazo[1,2-a]pyrazin-8-yl]amino]benzoyl]-N-[(3S)-pyrrolidin-3-yl]piperazine-1-carboxamide;
N-(3-aminopropyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
4-[2-chloro-4-[[3-[3-(trifluoromethyl)-1H-pyrazol-4-yl]imidazo[1,2-a]pyrazin-8-yl]amino]benzoyl]-N-[(3R)-pyrrolidin-3-yl]piperazine-1-carboxamide;
N-(2-aminoethyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
N-(azetidin-3-ylmethyl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
N-(azetidin-3-yl)-4-[2-chloro-4-[[3-[3-(trifluoromethyl)-1H-pyrazol-4-yl]imidazo[1,2-a]pyrazin-8-yl]amino]benzoyl]piperazine-1-carboxamide;
4-amino-1-[4-[2-chloro-4-[[3-[3-(trifluoromethyl)-1H-pyrazol-4-yl]imidazo[1,2-a]pyrazin-8-yl]amino]benzoyl]piperazin-1-yl]butan-1-one;
3-amino-1-[4-[2-chloro-4-[[3-[3-(trifluoromethyl)-1H-pyrazol-4-yl]imidazo[1,2-a]pyrazin-8-yl]amino]benzoyl]piperazin-1-yl]propan-1-one;
[2-chloro-4-[[3-[3-(trifluoromethyl)-1H-pyrazol-4-yl]imidazo[1,2-a]pyrazin-8-yl]amino]phenyl]-[4-(piperidine-4-carbonyl)piperazin-1-yl]methanone;
2-chloro-N,N-dimethyl-4-[[3-[3-(trifluoromethyl)-1H-pyrazol-4-yl]imidazo[1,2-a]pyrazin-8-yl]amino]benzamide;
N-(3-aminocyclobutyl)-2-chloro-4-[[3-[3-(trifluoromethyl)-1H-pyrazol-4-yl]imidazo[1,2-a]pyrazin-8-yl]amino]benzamide;
2-chloro-4-[[3-[3-(trifluoromethyl)-1H-pyrazol-4-yl]imidazo[1,2-a]pyrazin-8-yl]amino]benzamide;
N-(4-aminocyclohexyl)-2-chloro-4-[[3-[3-(trifluoromethyl)-1H-pyrazol-4-yl]imidazo[1,2-a]pyrazin-8-yl]amino]benzamide;
N-(azetidin-3-yl)-2-chloro-4-[[3-[3-(trifluoromethyl)-1H-pyrazol-4-yl]imidazo[1,2-a]pyrazin-8-yl]amino]benzamide;
2-chloro-N-[rac-(1R,3R)-3-aminocyclopentyl]-4-[[3-[3-(trifluoromethyl)-1H-pyrazol-4-yl]imidazo[1,2-a]pyrazin-8-yl]amino]benzamide;
N-(azetidin-3-yl)-4-[2-methyl-4-[[3-[3-(trifluoromethyl)-1H-pyrazol-4-yl]imidazo[1,2-a]pyrazin-8-yl]amino]benzoyl]piperazine-1-carboxamide;
N-(azetidin-3-ylmethyl)-4-[2-methyl-4-[[3-[3-(trifluoromethyl)-1H-pyrazol-4-yl]imidazo[1,2-a]pyrazin-8-yl]amino]benzoyl]piperazine-1-carboxamide;
2-chloro-N-piperidin-4-yl-4-[[3-[3-(trifluoromethyl)-1H-pyrazol-4-yl]imidazo[1,2-a]pyrazin-8-yl]amino]benzamide;
2-chloro-N-piperidin-3-yl-4-[[3-[3-(trifluoromethyl)-1H-pyrazol-4-yl]imidazo[1,2-a]pyrazin-8-yl]amino]benzamide;
4-[2-methyl-4-[[3-[3-(trifluoromethyl)-1H-pyrazol-4-yl]imidazo[1,2-a]pyrazin-8-yl]amino]benzoyl]-N-[(3S)-pyrrolidin-3-yl]piperazine-1-carboxamide;
4-[2-methyl-4-[[3-[3-(trifluoromethyl)-1H-pyrazol-4-yl]imidazo[1,2-a]pyrazin-8-yl]amino]benzoyl]-N-[(3R)-pyrrolidin-3-yl]piperazine-1-carboxamide;
N-(3-aminopropyl)-4-[2-methyl-4-[[3-[3-(trifluoromethyl)-1H-pyrazol-4-yl]imidazo[1,2-a]pyrazin-8-yl]amino]benzoyl]piperazine-1-carboxamide;
3-amino-1-[4-[2-methyl-4-[[3-[3-(trifluoromethyl)-1H-pyrazol-4-yl]imidazo[1,2-a]pyrazin-8-yl]amino]benzoyl]piperazin-1-yl]propan-1-one;
4-[4-[[3-[1-(cyanomethyl)-3-(trifluoromethyl)pyrazol-4-yl]imidazo[1,2-a]pyrazin-8-yl]amino]-2-methylbenzoyl]-N-[(3R,4R)-4-hydroxypyrrolidin-3-yl]piperazine-1-carboxamide;
4-amino-1-[4-[2-methyl-4-[[3-[3-(trifluoromethyl)-1H-pyrazol-4-yl]imidazo[1,2-a]pyrazin-8-yl]amino]benzoyl]piperazin-1-yl]butan-1-one;
2-[4-[8-[4-[4-[(3R,4R)-3-hydroxypiperidine-4-carbonyl]piperazine-1-carbonyl]-3-methylanilino]imidazo[1,2-a]pyrazin-3-yl]-3-(trifluoromethyl)pyrazol-1-yl]acetonitrile;
2-[4-[8-[4-[4-[(2S,4R)-4-hydroxypyrrolidine-2-carbonyl]piperazine-1-carbonyl]-3-methylanilino]imidazo[1,2-a]pyrazin-3-yl]-3-(trifluoromethyl)pyrazol-1-yl]acetonitrile;
2-[4-[8-[4-[4-[(3S,4S)-3-hydroxypiperidine-4-carbonyl]piperazine-1-carbonyl]-3-methylanilino]imidazo[1,2-a]pyrazin-3-yl]-3-(trifluoromethyl)pyrazol-1-yl]acetonitrile;
2-[4-[8-[4-[4-[(2S,4S)-4-hydroxy-4-methylpyrrolidine-2-carbonyl]piperazine-1-carbonyl]-3-methylanilino]imidazo[1,2-a]pyrazin-3-yl]-3-(trifluoromethyl)pyrazol-1-yl]acetonitrile;
2-[4-[8-[4-[4-(4-hydroxypiperidine-4-carbonyl)piperazine-1-carbonyl]-3-methylanilino]imidazo[1,2-a]pyrazin-3-yl]-3-(trifluoromethyl)pyrazol-1-yl]acetonitrile;
2-[4-[8-[4-[4-[(2R,4S)-4-hydroxypyrrolidine-2-carbonyl]piperazine-1-carbonyl]-3-methylanilino]imidazo[1,2-a]pyrazin-3-yl]-3-(trifluoromethyl)pyrazol-1-yl]acetonitrile;
N-(2-aminoethyl)-4-[2-methyl-4-[[3-[3-(trifluoromethyl)-1H-pyrazol-4-yl]imidazo[1,2-a]pyrazin-8-yl]amino]benzoyl]piperazine-1-carboxamide;
(2S,4R)—N-[3-[[2-chloro-4-[[3-[3-(trifluoromethyl)-1H-pyrazol-4-yl]imidazo[1,2-a]pyrazin-8-yl]amino]benzoyl]amino]propyl]-4-hydroxypyrrolidine-2-carboxamide;
N-[2-(2-aminoethoxy)ethyl]-4-[[3-[3-(difluoromethyl)-1H-pyrazol-4-yl]imidazo[1,2-a]pyrazin-8-yl]amino]-2-ethylbenzamide;
[2-chloro-4-[[3-[3-(trifluoromethyl)-1H-pyrazol-4-yl]imidazo[1,2-a]pyrazin-8-yl]amino]phenyl]-piperazin-1-ylmethanone;
(2S,4R)—N-[2-[[2-ethyl-4-[[3-[3-(trifluoromethyl)-1H-pyrazol-4-yl]imidazo[1,2-a]pyrazin-8-yl]amino]benzoyl]amino]ethyl]-4-hydroxypyrrolidine-2-carboxamide;
(2S,4R)—N-[2-[[2-chloro-4-[[3-[3-(trifluoromethyl)-1H-pyrazol-4-yl]imidazo[1,2-a]pyrazin-8-yl]amino]benzoyl]amino]ethyl]-4-hydroxypyrrolidine-2-carboxamide;
(2S,4R)—N-[3-[[2-ethyl-4-[[3-[3-(trifluoromethyl)-1H-pyrazol-4-yl]imidazo[1,2-a]pyrazin-8-yl]amino]benzoyl]amino]propyl]-4-hydroxypyrrolidine-2-carboxamide;
(2S,4S)-4-ethyl-N-[2-[[2-ethyl-4-[[3-[3-(trifluoromethyl)-1H-pyrazol-4-yl]imidazo[1,2-a]pyrazin-8-yl]amino]benzoyl]amino]ethyl]-4-hydroxypyrrolidine-2-carboxamide;
[4-(3-hydroxypiperidine-3-carbonyl)piperazin-1-yl]-[2-methyl-4-[[3-[3-(trifluoromethyl)-1H-pyrazol-4-yl]imidazo[1,2-a]pyrazin-8-yl]amino]phenyl]methanone;
(2S,4R)—N-[3-[[2-ethyl-4-[[3-[1-methyl-3-(trifluoromethyl)pyrazol-4-yl]imidazo[1,2-a]pyrazin-8-yl]amino]benzoyl]amino]propyl]-4-hydroxypyrrolidine-2-carboxamide;
[2-methyl-4-[[3-[3-(trifluoromethyl)-1H-pyrazol-4-yl]imidazo[1,2-a]pyrazin-8-yl]amino]phenyl]-[4-[rac-(3R,5R)-5-hydroxypiperidine-3-carbonyl]piperazin-1-yl]methanone;
4-[2-chloro-4-[[3-[3-(trifluoromethyl)-1H-pyrazol-4-yl]imidazo[1,2-a]pyrazin-8-yl]amino]benzoyl]-N-[(3S,4S)-4-hydroxypyrrolidin-3-yl]piperazine-1-carboxamide;
4-[2-chloro-4-[[3-[3-(trifluoromethyl)-1H-pyrazol-4-yl]imidazo[1,2-a]pyrazin-8-yl]amino]benzoyl]-N-[(3R,4R)-4-hydroxypyrrolidin-3-yl]piperazine-1-carboxamide;
(2S,4R)-4-hydroxy-N-[3-[[2-methyl-4-[[3-[3-(trifluoromethyl)-1H-pyrazol-4-yl]imidazo[1,2-a]pyrazin-8-yl]amino]benzoyl]amino]propyl]pyrrolidine-2-carboxamide;
[2-chloro-4-[[3-[3-(trifluoromethyl)-1H-pyrazol-4-yl]imidazo[1,2-a]pyrazin-8-yl]amino]phenyl]-[4-[(2S,4S)-4-hydroxy-4-methylpyrrolidine-2-carbonyl]piperazin-1-yl]methanone;
(2S,4R)-4-hydroxy-N-[2-[[2-methyl-4-[[3-[3-(trifluoromethyl)-1H-pyrazol-4-yl]imidazo[1,2-a]pyrazin-8-yl]amino]benzoyl]amino]ethyl]pyrrolidine-2-carboxamide;
(2S,4R)—N-[2-[[2-ethyl-4-[[3-[1-methyl-3-(trifluoromethyl)pyrazol-4-yl]imidazo[1,2-a]pyrazin-8-yl]amino]benzoyl]amino]ethyl]-4-hydroxypyrrolidine-2-carboxamide;
(2S,4S)-4-ethyl-N-[2-[[2-ethyl-4-[[3-[1-methyl-3-(trifluoromethyl)pyrazol-4-yl]imidazo[1,2-a]pyrazin-8-yl]amino]benzoyl]amino]ethyl]-4-hydroxypyrrolidine-2-carboxamide;
(2S,4S)—N-[2-[[2-ethyl-4-[[3-[1-methyl-3-(trifluoromethyl)pyrazol-4-yl]imidazo[1,2-a]pyrazin-8-yl]amino]benzoyl]amino]ethyl]-4-hydroxy-4-methylpyrrolidine-2-carboxamide;
[2-chloro-4-[[3-[3-(trifluoromethyl)-1H-pyrazol-4-yl]imidazo[1,2-a]pyrazin-8-yl]amino]phenyl]-[4-[(2S,4R)-4-hydroxypyrrolidine-2-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-(4-hydroxypiperidine-4-carbonyl)piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(3R,4R)-3-hydroxypiperidine-4-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(3S,4S)-3-hydroxypiperidine-4-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(2R,4S)-4-hydroxypyrrolidine-2-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(3R,4S)-3-hydroxypiperidine-4-carbonyl]piperazin-1-yl]methanone;
[2-chloro-4-[[3-[3-(trifluoromethyl)-1H-pyrazol-4-yl]imidazo[1,2-a]pyrazin-8-yl]amino]phenyl]-[4-[(3S,4R)-3-hydroxypiperidine-4-carbonyl]piperazin-1-yl]methanone;
(2S,4R)-4-hydroxy-N-[3-[[2-methyl-4-[[3-[1-methyl-3-(trifluoromethyl)pyrazol-4-yl]imidazo[1,2-a]pyrazin-8-yl]amino]benzoyl]amino]propyl]pyrrolidine-2-carboxamide;
(2S,4R)-4-hydroxy-N-[2-[[2-methyl-4-[[3-[1-methyl-3-(trifluoromethyl)pyrazol-4-yl]imidazo[1,2-a]pyrazin-8-yl]amino]benzoyl]amino]ethyl]pyrrolidine-2-carboxamide;
N-[(3R,4R)-4-hydroxypyrrolidin-3-yl]-4-[2-methyl-4-[[3-[3-(trifluoromethyl)-1H-pyrazol-4-yl]imidazo[1,2-a]pyrazin-8-yl]amino]benzoyl]piperazine-1-carboxamide;
N-[(3S,4S)-4-hydroxypyrrolidin-3-yl]-4-[2-methyl-4-[[3-[3-(trifluoromethyl)-1H-pyrazol-4-yl]imidazo[1,2-a]pyrazin-8-yl]amino]benzoyl]piperazine-1-carboxamide;
[4-[(2S,4R)-4-hydroxypyrrolidine-2-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-(4-hydroxypiperidine-4-carbonyl)piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-[(3R,4R)-3-hydroxypiperidine-4-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-[(2S,4S)-4-hydroxy-4-methylpyrrolidine-2-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-[(3S,4S)-3-hydroxypiperidine-4-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-[(3S,4R)-3-hydroxypiperidine-4-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-[(3R,4S)-3-hydroxypiperidine-4-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[4-[(2R,4S)-4-hydroxypyrrolidine-2-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[2-methyl-4-[[3-[3-(trifluoromethyl)-1H-pyrazol-4-yl]imidazo[1,2-a]pyrazin-8-yl]amino]phenyl]-[4-[rac-(3R,4R)-4-hydroxypiperidine-3-carbonyl]piperazin-1-yl]methanone;
[2-methyl-4-[[3-[3-(trifluoromethyl)-1H-pyrazol-4-yl]imidazo[1,2-a]pyrazin-8-yl]amino]phenyl]-[4-[rac-(3R,4S)-4-hydroxypiperidine-3-carbonyl]piperazin-1-yl]methanone;
N-[(3R,4R)-4-hydroxypyrrolidin-3-yl]-4-[2-methyl-4-[[3-[1-methyl-3-(trifluoromethyl)pyrazol-4-yl]imidazo[1,2-a]pyrazin-8-yl]amino]benzoyl]piperazine-1-carboxamide;
N-[(3S,4S)-4-hydroxypyrrolidin-3-yl]-4-[2-methyl-4-[[3-[1-methyl-3-(trifluoromethyl)pyrazol-4-yl]imidazo[1,2-a]pyrazin-8-yl]amino]benzoyl]piperazine-1-carboxamide;
[4-(4-hydroxypiperidine-4-carbonyl)piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(2S,4S)-4-hydroxy-4-methylpyrrolidine-2-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(2S,4R)-4-hydroxypyrrolidine-2-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(3R,4R)-3-hydroxypiperidine-4-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(3S,4S)-3-hydroxypiperidine-4-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(2R,4S)-4-hydroxypyrrolidine-2-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(3S,4R)-3-hydroxypiperidine-4-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(3R,4S)-3-hydroxypiperidine-4-carbonyl]piperazin-1-yl]-[2-methyl-4-[[3-[1-methyl-3-(trifluoromethyl)pyrazol-4-yl]imidazo[1,2-a]pyrazin-8-yl]amino]phenyl]methanone;
[4-[(2S,4R)-4-hydroxypyrrolidine-2-carbonyl]piperazin-1-yl]-[2-methyl-4-[[3-[3-(trifluoromethyl)-1H-pyrazol-4-yl]imidazo[1,2-a]pyrazin-8-yl]amino]phenyl]methanone;
[2-methyl-4-[[3-[3-(trifluoromethyl)-1H-pyrazol-4-yl]imidazo[1,2-a]pyrazin-8-yl]amino]phenyl]-piperazin-1-ylmethanone;
2-(4-(8-((4-(4-((1R,5S,6r)-3-azabicyclo[3.1.0]hexane-6-carbonyl)piperazine-1-carbonyl)-3-chlorophenyl)amino)imidazo[1,2-a]pyrazin-3-yl)-3-(trifluoromethyl)-1H-pyrazol-1-yl)acetonitrile;
N-(3-((1s,3s)-3-aminocyclobutane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1R,3S)-3-(12-azaneyl)cyclopentane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1S,3R)-3-aminocyclopentane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1S,3R)-3-aminocyclopentane-1-carboxamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-(4-((3-(1-(cyanomethyl)-3-(trifluoromethyl)-1H-pyrazol-4-yl)imidazo[1,2-a]pyrazin-8-yl)amino)-2-ethylbenzamido)ethyl)piperidine-4-carboxamide;
N-(2-((1r,3r)-3-aminocyclobutane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1r,3r)-3-aminocyclobutane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(S)-3-amino-N-(2-(4-((3-(1-(2,2-difluoroethyl)-3-(trifluoromethyl)-1H-pyrazol-4-yl)imidazo[1,2-a]pyrazin-8-yl)amino)-2-ethylbenzamido)ethyl)pyrrolidine-1-carboxamide;
N-(2-((1R,3R)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-((1S,3S)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1S,3S)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(S)—N-(2-(2-aminopropanamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1R,3S)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1S,3R)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-((1S,3R)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1R,3S)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
cis-N-(2-((1s,3s)-3-aminocyclobutane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
4-[[3-[1-(cyanomethyl)-3-(trifluoromethyl)pyrazol-4-yl]imidazo[1,2-a]pyrazin-8-yl]amino]-2-ethyl-N-(2-oxo-2-piperazin-1-yl-ethyl)benzamide;
N-[2-(4-amino-1-piperidyl)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
2-chloro-4-[[3-[1-(cyanomethyl)-3-(trifluoromethyl)pyrazol-4-yl]imidazo[1,2-a]pyrazin-8-yl]amino]-N-(2-oxo-2-piperazin-1-yl-ethyl)benzamide;
N-[2-(4-amino-1-piperidyl)-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
N-[2-(4-amino-1-piperidyl)-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
N-[2-[(3R)-3-aminopyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
4-[[3-[1-(cyanomethyl)-3-(trifluoromethyl)pyrazol-4-yl]imidazo[1,2-a]pyrazin-8-yl]amino]-N-[2-(2,6-diazaspiro[3.3]heptan-2-yl)-2-oxo-ethyl]-2-ethyl-benzamide;
N-[2-(3-amino-1-piperidyl)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
4-[[3-[1-(cyanomethyl)-3-(trifluoromethyl)pyrazol-4-yl]imidazo[1,2-a]pyrazin-8-yl]amino]-2-ethyl-N-[2-(3-methylpiperazin-1-yl)-2-oxo-ethyl]benzamide;
N-[2-(6-amino-2-azaspiro[3.3]heptan-2-yl)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
N-[2-[3-(aminomethyl)pyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
N-[2-[2-(aminomethyl)pyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
N-[2-(3-azabicyclo[3.1.0]hexan-6-ylamino)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
4-[[3-[1-(cyanomethyl)-3-(trifluoromethyl)pyrazol-4-yl]imidazo[1,2-a]pyrazin-8-yl]amino]-N-[2-(2,5-diazabicyclo[2.2.1]heptan-2-yl)-2-oxo-ethyl]-2-ethyl-benzamide;
N-(azetidin-3-ylmethyl)-2-ethyl-4-((3-(3-(trifluoromethyl)-1H-pyrazol-4-yl)imidazo[1,2-a]pyrazin-8-yl)amino)benzamide;
2-ethyl-N-(2-azaspiro[3.3]heptan-6-yl)-4-((3-(3-(trifluoromethyl)-1H-pyrazol-4-yl)imidazo[1,2-a]pyrazin-8-yl)amino)benzamide;
N-(azetidin-3-yl)-2-ethyl-4-((3-(3-(trifluoromethyl)-1H-pyrazol-4-yl)imidazo[1,2-a]pyrazin-8-yl)amino)benzamide;
(4-((1R,5S,6r)-3-azabicyclo[3.1.0]hexane-6-carbonyl)piperazin-1-yl(2-chloro-4-((3-(3-(trifluoromethyl)-1H-pyrazol-4-yl)imidazo[1,2-a]pyrazin-8-yl)amino)phenyl)methanone;
(R)-3-amino-N-(2-(2-ethyl-4-((3-(3-(trifluoromethyl)-1H-pyrazol-4-yl)imidazo[1,2-a]pyrazin-8-yl)amino)benzamido)ethyl)pyrrolidine-1-carboxamide;
((3-aminopropyl)imino)(methyl)(2-methyl-4-((3-(3-(trifluoromethyl)-1H-pyrazol-4-yl)imidazo[1,2-a]pyrazin-8-yl)amino)phenyl)-16-sulfanone;
(4-((1R,5S,6r)-3-azabicyclo[3.1.0]hexane-6-carbonyl)piperazin-1-yl)(2-chloro-4-((3-(1-(2,2-difluoroethyl)-3-(trifluoromethyl)-1H-pyrazol-4-yl)imidazo[1,2-a]pyrazin-8-yl)amino)phenyl)methanone;
(R)-3-amino-N-(2-(4-((3-(1-(cyanomethyl)-3-(trifluoromethyl)-1H-pyrazol-4-yl)imidazo[1,2-a]pyrazin-8-yl)amino)-2-ethylbenzamido)ethyl)pyrrolidine-1-carboxamide;
N-[2-(azetidin-3-ylmethylamino)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
4-[[3-[1-(cyanomethyl)-3-(trifluoromethyl)pyrazol-4-yl]imidazo[1,2-a]pyrazin-8-yl]amino]-2-ethyl-N-[2-oxo-2-(pyrrolidin-2-ylmethylamino)ethyl]benzamide;
4-[[3-[1-(cyanomethyl)-3-(trifluoromethyl)pyrazol-4-yl]imidazo[1,2-a]pyrazin-8-yl]amino]-2-ethyl-N-[2-oxo-2-(pyrrolidin-3-ylmethylamino)ethyl]benzamide;
4-[[3-[1-(cyanomethyl)-3-(trifluoromethyl)pyrazol-4-yl]imidazo[1,2-a]pyrazin-8-yl]amino]-2-ethyl-N-[2-oxo-2-(4-piperidylmethylamino)ethyl]benzamide;
4-[[3-[1-(cyanomethyl)-3-(trifluoromethyl)pyrazol-4-yl]imidazo[1,2-a]pyrazin-8-yl]amino]-2-ethyl-N-[2-[(4-hydroxy-4-piperidyl)methylamino]-2-oxo-ethyl]benzamide;
N-[2-(2,6-diazaspiro[3.3]heptan-2-yl)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
N-[2-[3-(aminomethyl)pyrrolidin-1-yl]-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
N-[2-[2-(aminomethyl)pyrrolidin-1-yl]-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
N-[2-(3-azabicyclo[3.1.0]hexan-6-ylamino)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
N-[2-(azetidin-2-ylmethylamino)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
N-(3-((1s,3s)-3-aminocyclobutane-1-carboxamido)propyl)-2-ethyl-4-((3-(3-(trifluoromethyl)-1H-pyrazol-4-yl)imidazo[1,2-a]pyrazin-8-yl)amino)benzamide;
N-(3-((1r,3r)-3-aminocyclobutane-1-carboxamido)propyl)-2-ethyl-4-((3-(3-(trifluoromethyl)-1H-pyrazol-4-yl)imidazo[1,2-a]pyrazin-8-yl)amino)benzamide;
N-(3-((1R,3S)-3-aminocyclopentane-1-carboxamido)propyl)-2-ethyl-4-((3-(3-(trifluoromethyl)-1H-pyrazol-4-yl)imidazo[1,2-a]pyrazin-8-yl)amino)benzamide;
N-(3-((1S,3R)-3-aminocyclopentane-1-carboxamido)propyl)-2-ethyl-4-((3-(3-(trifluoromethyl)-1H-pyrazol-4-yl)imidazo[1,2-a]pyrazin-8-yl)amino)benzamide;
N-(2-(3-aminopropanamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-(4-aminobutanamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-[2-(2,5-diazabicyclo[2.2.1]heptan-2-yl)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
N-((1s,3s)-3-aminocyclobutyl)-4-((3-(1-(2,2-difluoroethyl)-3-(difluoromethyl)-1H-pyrazol-4-yl)imidazo[1,2-a]pyrazin-8-yl)amino)-2-ethylbenzamide;
N-(2-(4-aminobutanamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1s,3s)-3-aminocyclobutane-1-carboxamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(3-((1r,3r)-3-aminocyclobutane-1-carboxamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(3-((1R,3S)-3-aminocyclopentane-1-carboxamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-[2-(3-amino-1-piperidyl)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-(3-methylpiperazin-1-yl)-2-oxo-ethyl]benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-[(3-hydroxyazetidin-3-yl)methylamino]-2-oxo-ethyl]benzamide;
N-[2-(azetidin-3-ylmethylamino)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-oxo-2-(pyrrolidin-2-ylmethylamino)ethyl]benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-oxo-2-(pyrrolidin-3-ylmethylamino)ethyl]benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-oxo-2-(4-piperidylmethylamino)ethyl]benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-[(4-hydroxy-4-piperidyl)methylamino]-2-oxo-ethyl]benzamide;
(2S,4R)—N-((1s,3R)-3-(4-((3-(1-(2,2-difluoroethyl)-3-(difluoromethyl)-1H-pyrazol-4-yl)imidazo[1,2-a]pyrazin-8-yl)amino)-2-ethylbenzamido)cyclobutyl)-4-hydroxypyrrolidine-2-carboxamide;
N-(2-(2-ethyl-4-((3-(3-(trifluoromethyl)-1H-pyrazol-4-yl)imidazo[1,2-a]pyrazin-8-yl)amino)benzamido)ethyl)piperidine-4-carboxamide;
N-(2-(4-((3-(1-(2,2-difluoroethyl)-3-(trifluoromethyl)-1H-pyrazol-4-yl)imidazo[1,2-a]pyrazin-8-yl)amino)-2-ethylbenzamido)ethyl)piperidine-4-carboxamide;
(4-((3-(1-(2,2-difluoroethyl)-3-(trifluoromethyl)-1H-pyrazol-4-yl)imidazo[1,2-a]pyrazin-8-yl)amino)-2-ethylphenyl)(2,6-diazaspiro[3.4]octan-6-yl)methanone;
(3-aminoazetidin-1-yl)(4-((3-(1-(2,2-difluoroethyl)-3-(trifluoromethyl)-1H-pyrazol-4-yl)imidazo[1,2-a]pyrazin-8-yl)amino)-2-ethylphenyl)methanone;
N-(azetidin-3-ylmethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(3-amino-2-(chloromethyl)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(4-((3-(1-(2,2-difluoroethyl)-3-(trifluoromethyl)-1H-pyrazol-4-yl)imidazo[1,2-a]pyrazin-8-yl)amino)-2-ethylphenyl)(1,6-diazaspiro[3.3]heptan-1-yl)methanone;
(2-(aminomethyl)-2-(chloromethyl)azetidin-1-yl)(4-((3-(1-(2,2-difluoroethyl)-3-(trifluoromethyl)-1H-pyrazol-4-yl)imidazo[1,2-a]pyrazin-8-yl)amino)-2-ethylphenyl)methanone;
(2,5-diazabicyclo[2.2.1]heptan-2-yl)(4-((3-(1-(2,2-difluoroethyl)-3-(trifluoromethyl)-1H-pyrazol-4-yl)imidazo[1,2-a]pyrazin-8-yl)amino)-2-ethylphenyl)methanone;
N-(3-azabicyclo[3.1.0]hexan-6-yl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
4-((3-(1-(2,2-difluoroethyl)-3-(trifluoromethyl)-1H-pyrazol-4-yl)imidazo[1,2-a]pyrazin-8-yl)amino)-2-ethyl-N-(piperidin-3-yl)benzamide;
(4-((3-(1-(2,2-difluoroethyl)-3-(trifluoromethyl)-1H-pyrazol-4-yl)imidazo[1,2-a]pyrazin-8-yl)amino)-2-ethylphenyl)(1,6-diazaspiro[3.3]heptan-6-yl)methanone;
N-(azetidin-3-yl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-(3-aminopropanamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1r,3r)-3-aminocyclobutane-1-carboxamido)ethyl)-2-ethyl-4-((3-(3-(trifluoromethyl)-1H-pyrazol-4-yl)imidazo[1,2-a]pyrazin-8-yl)amino)benzamide;
4-amino-N-(2-(2-ethyl-4-((3-(3-(trifluoromethyl)-1H-pyrazol-4-yl)imidazo[1,2-a]pyrazin-8-yl)amino)benzamido)ethyl)piperidine-1-carboxamide;
N-(2-(3-(3-aminopropyl)ureido)ethyl)-2-ethyl-4-((3-(3-(trifluoromethyl)-1H-pyrazol-4-yl)imidazo[1,2-a]pyrazin-8-yl)amino)benzamide;
1-(4-(2-chloro-4-((3-(3-(trifluoromethyl)-1H-pyrazol-4-yl)imidazo[1,2-a]pyrazin-8-yl)amino)benzoyl)piperazin-1-yl)-2-(piperidin-4-yl)ethan-1-one;
(4-(2-aminoethyl)piperazin-1-yl)(2-chloro-4-((3-(3-(trifluoromethyl)-1H-pyrazol-4-yl)imidazo[1,2-a]pyrazin-8-yl)amino)phenyl)methanone;
2-(4-(8-((3-chloro-4-(4-(2-(piperidin-4-yl)acetyl)piperazine-1-carbonyl)phenyl)amino)imidazo[1,2-a]pyrazin-3-yl)-3-(trifluoromethyl)-1H-pyrazol-1-yl)acetonitrile;
2-(4-(8-((4-(4-((1R,3R)-3-aminocyclopentane-1-carbonyl)piperazine-1-carbonyl)-3-chlorophenyl)amino)imidazo[1,2-a]pyrazin-3-yl)-3-(trifluoromethyl)-1H-pyrazol-1-yl)acetonitrile;
(4-((1R,3R)-3-aminocyclopentane-1-carbonyl)piperazin-1-yl)(2-chloro-4-((3-(1-(2,2-difluoroethyl)-3-(trifluoromethyl)-1H-pyrazol-4-yl)imidazo[1,2-a]pyrazin-8-yl)amino)phenyl)methanone;
(4-((1S,3S)-3-aminocyclopentane-1-carbonyl)piperazin-1-yl)(2-chloro-4-((3-(3-(trifluoromethyl)-1H-pyrazol-4-yl)imidazo[1,2-a]pyrazin-8-yl)amino)phenyl)methanone;
(S)-3-amino-N-(2-(2-ethyl-4-((3-(3-(trifluoromethyl)-1H-pyrazol-4-yl)imidazo[1,2-a]pyrazin-8-yl)amino)benzamido)ethyl)pyrrolidine-1-carboxamide;
(S)-3-amino-N-(2-(4-((3-(1-(cyanomethyl)-3-(trifluoromethyl)-1H-pyrazol-4-yl)imidazo[1,2-a]pyrazin-8-yl)amino)-2-ethylbenzamido)ethyl)pyrrolidine-1-carboxamide;
1-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)-2-(piperidin-4-yl)ethan-1-one;
4-(2-chloro-4-((3-(1-(cyanomethyl)-3-(trifluoromethyl)-1H-pyrazol-4-yl)imidazo[1,2-a]pyrazin-8-yl)amino)benzoyl)-N-((3-hydroxyazetidin-3-yl)methyl)piperazine-1-carboxamide;
4-(2-chloro-4-((3-(1-(2,2-difluoroethyl)-3-(trifluoromethyl)-1H-pyrazol-4-yl)imidazo[1,2-a]pyrazin-8-yl)amino)benzoyl)-N-((3-hydroxyazetidin-3-yl)methyl)piperazine-1-carboxamide;
N-(azetidin-3-yl)-4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazine-1-carboxamide;
2-(4-(8-((4-(4-((1S,3S)-3-aminocyclopentane-1-carbonyl)piperazine-1-carbonyl)-3-chlorophenyl)amino)imidazo[1,2-a]pyrazin-3-yl)-3-(trifluoromethyl)-1H-pyrazol-1-yl)acetonitrile;
(4-((1S,3S)-3-aminocyclopentane-1-carbonyl)piperazin-1-yl)(2-chloro-4-((3-(1-(2,2-difluoroethyl)-3-(trifluoromethyl)-1H-pyrazol-4-yl)imidazo[1,2-a]pyrazin-8-yl)amino)phenyl)methanone;
N-((1s,3s)-3-aminocyclobutyl)-4-(2-chloro-4-((3-(1-(cyanomethyl)-3-(trifluoromethyl)-1H-pyrazol-4-yl)imidazo[1,2-a]pyrazin-8-yl)amino)benzoyl)piperazine-1-carboxamide;
N-((1s,3s)-3-aminocyclobutyl)-4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazine-1-carboxamide;
N-[2-[[(1R,3R)-3-aminocyclopentanecarbonyl]amino]ethyl]-2-ethyl-4-[[3-[3-(trifluoromethyl)-1H-pyrazol-4-yl]imidazo[1,2-a]pyrazin-8-yl]amino]benzamide;
N-(2-((1R,3R)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1S,3S)-3-aminocyclopentane-1-carboxamido)ethyl)-2-ethyl-4-((3-(3-(trifluoromethyl)-1H-pyrazol-4-yl)imidazo[1,2-a]pyrazin-8-yl)amino)benzamide;
4-((3-(1-((1-(cyanomethyl)-1H-pyrazol-3-yl)methyl)-3-(trifluoromethyl)-1H-pyrazol-4-yl)imidazo[1,2-a]pyrazin-8-yl)amino)-2-ethyl-N-methylbenzamide;
2-chloro-4-((3-(1-((1-(cyanomethyl)-1H-pyrazol-3-yl)methyl)-3-(trifluoromethyl)-1H-pyrazol-4-yl)imidazo[1,2-a]pyrazin-8-yl)amino)-N-methylbenzamide;
2-chloro-4-((3-(1-(2-(dimethylamino)ethyl)-3-(trifluoromethyl)-1H-pyrazol-4-yl)imidazo[1,2-a]pyrazin-8-yl)amino)-N-methylbenzamide;
4-((3-(1-(1-benzylpiperidin-4-yl)-3-(trifluoromethyl)-1H-pyrazol-4-yl)imidazo[1,2-a]pyrazin-8-yl)amino)-2-chloro-N-methylbenzamide;
4-((3-(1-((1-benzylpyrrolidin-3-yl)methyl)-3-(trifluoromethyl)-1H-pyrazol-4-yl)imidazo[1,2-a]pyrazin-8-yl)amino)-2-chloro-N-methylbenzamide;
4-((3-(1-(3-(4-(aminomethyl)phenyl)propyl)-3-(trifluoromethyl)-1H-pyrazol-4-yl)imidazo[1,2-a]pyrazin-8-yl)amino)-2-chloro-N-methylbenzamide;
4-((3-(1-(3-(2-aminoethyl)benzyl)-3-(trifluoromethyl)-1H-pyrazol-4-yl)imidazo[1,2-a]pyrazin-8-yl)amino)-2-chloro-N-methylbenzamide;
N-[4-[N-(3-aminopropyl)-S-methyl-sulfonimidoyl]-3-methyl-phenyl]-3-[1-(cyclopropylmethyl)-3-(trifluoromethyl)pyrazol-4-yl]imidazo[1,2-a]pyrazin-8-amine;
2-[4-[8-[4-[N-(3-aminopropyl)-S-methyl-sulfonimidoyl]-3-methyl-anilino]imidazo[1,2-a]pyrazin-3-yl]-3-(trifluoromethyl)pyrazol-1-yl]acetonitrile;
N-(2-aminoethyl)-2-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)acetamide;
N-(azetidin-3-yl)-2-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)acetamide;
(S)-2-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)-N-(pyrrolidin-3-yl)acetamide;
(R)-2-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)-N-(pyrrolidin-3-yl)acetamide;
N-(2-aminoethyl)-2-(4-(2-chloro-4-((3-(1-(cyanomethyl)-3-(trifluoromethyl)-1H-pyrazol-4-yl)imidazo[1,2-a]pyrazin-8-yl)amino)benzoyl)piperazin-1-yl)acetamide;
N-(3-aminopropyl)-2-(4-(2-chloro-4-((3-(1-(cyanomethyl)-3-(trifluoromethyl)-1H-pyrazol-4-yl)imidazo[1,2-a]pyrazin-8-yl)amino)benzoyl)piperazin-1-yl)acetamide;
N-(azetidin-3-yl)-2-(4-(2-chloro-4-((3-(1-(cyanomethyl)-3-(trifluoromethyl)-1H-pyrazol-4-yl)imidazo[1,2-a]pyrazin-8-yl)amino)benzoyl)piperazin-1-yl)acetamide;
(S)-2-(4-(2-chloro-4-((3-(1-(cyanomethyl)-3-(trifluoromethyl)-1H-pyrazol-4-yl)imidazo[1,2-a]pyrazin-8-yl)amino)benzoyl)piperazin-1-yl)-N-(pyrrolidin-3-yl)acetamide;
N-(3-aminopropyl)-2-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)acetamide;
2-(4-(8-((3-chloro-4-(4-(2-oxo-2-(piperazin-1-yl)ethyl)piperazine-1-carbonyl)phenyl)amino)imidazo[1,2-a]pyrazin-3-yl)-3-(trifluoromethyl)-1H-pyrazol-1-yl)acetonitrile;
2-(4-(2-chloro-4-((3-(1-(2,2-difluoroethyl)-3-(trifluoromethyl)-1H-pyrazol-4-yl)imidazo[1,2-a]pyrazin-8-yl)amino)benzoyl)piperazin-1-yl)-1-(piperazin-1-yl)ethan-1-one;
N-(2-(3-aminopropoxy)ethyl)-4-((3-(1-cyclobutyl-3-(trifluoromethyl)-1H-pyrazol-4-yl)imidazo[1,2-a]pyrazin-8-yl)amino)-2-ethylbenzamide;
N-(2-(3-aminopropoxy)ethyl)-2-ethyl-4-((3-(1-(2-fluoroethyl)-3-(trifluoromethyl)-1H-pyrazol-4-yl)imidazo[1,2-a]pyrazin-8-yl)amino)benzamide;
N-(2-(2-aminoacetamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-(3-aminopropoxy)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-(3-aminopropoxy)ethyl)-2-ethyl-4-((3-(3-(trifluoromethyl)-1H-pyrazol-4-yl)imidazo[1,2-a]pyrazin-8-yl)amino)benzamide;
N-(2-(2-aminoethoxy)ethyl)-2-ethyl-4-((3-(1-(3-methylbut-2-en-1-yl)-3-(trifluoromethyl)-1H-pyrazol-4-yl)imidazo[1,2-a]pyrazin-8-yl)amino)benzamide;
N-(6-aminohexyl)-4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamide;
(S)—N-(2-(2-aminopropanamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
2-ethyl-4-((3-(1-(2-fluoroethyl)-3-(trifluoromethyl)-1H-pyrazol-4-yl)imidazo[1,2-a]pyrazin-8-yl)amino)-N-(2-(2-(pyrrolidin-1-yl)ethoxy)ethyl)benzamide;
N-(2-(3-aminopropoxy)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
4-((3-(1-cyclobutyl-3-(trifluoromethyl)-1H-pyrazol-4-yl)imidazo[1,2-a]pyrazin-8-yl)amino)-2-ethyl-N-(2-(2-(pyrrolidin-1-yl)ethoxy)ethyl)benzamide;
N-(2-(2-aminoacetamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
4-((3-(1-(2,2-difluoroethyl)-3-(trifluoromethyl)-1H-pyrazol-4-yl)imidazo[1,2-a]pyrazin-8-yl)amino)-2-ethyl-N-(2-(2-(pyrrolidin-1-yl)ethoxy)ethyl)benzamide;
N-(2-(2-aminoethoxy)ethyl)-2-ethyl-4-((3-(1-(2-(trifluoromethoxy)ethyl)-3-(trifluoromethyl)-1H-pyrazol-4-yl)imidazo[1,2-a]pyrazin-8-yl)amino)benzamide;
N-((2-aminocyclopropyl)methyl)-4-((3-(1-(cyanomethyl)-3-(trifluoromethyl)-1H-pyrazol-4-yl)imidazo[1,2-a]pyrazin-8-yl)amino)-2-ethylbenzamide;
cis-N-(2-((1s,3s)-3-aminocyclobutane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(R)—N-(2-(2-aminopropanamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
(R)—N-(3-(2-aminopropanamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
(S)—N-(3-(4-((3-(1-(2,2-difluoroethyl)-3-(trifluoromethyl)-1H-pyrazol-4-yl)imidazo[1,2-a]pyrazin-8-yl)amino)-2-ethylbenzamido)propyl)pyrrolidine-2-carboxamide;
(S)—N-(3-(2-aminopropanamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(4-(4-((3-(1-allyl-3-(trifluoromethyl)-1H-pyrazol-4-yl)imidazo[1,2-a]pyrazin-8-yl)amino)-2-fluoro-6-methylbenzoyl)piperazin-1-yl)((2R,5S)-5-hydroxypyrrolidin-2-yl)methanone;
N-(3-(3-aminopropanamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(R)-3-amino-N-(3-(2-ethyl-4-((3-(3-(trifluoromethyl)-1H-pyrazol-4-yl)imidazo[1,2-a]pyrazin-8-yl)amino)benzamido)propyl)pyrrolidine-1-carboxamide;
(R)-3-amino-N-(3-(4-((3-(1-(2,2-difluoroethyl)-3-(trifluoromethyl)-1H-pyrazol-4-yl)imidazo[1,2-a]pyrazin-8-yl)amino)-2-ethylbenzamido)propyl)pyrrolidine-1-carboxamide;
(R)—N-(3-(2-aminopropanamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(S)—N-(3-(2-ethyl-4-((3-(3-(trifluoromethyl)-1H-pyrazol-4-yl)imidazo[1,2-a]pyrazin-8-yl)amino)benzamido)propyl)pyrrolidine-2-carboxamide;
(S)-(2-chloro-4-((3-(1-(2,2-difluoroethyl)-3-(trifluoromethyl)-1H-pyrazol-4-yl)imidazo[1,2-a]pyrazin-8-yl)amino)phenyl)(6-prolyl-2,6-diazaspiro[3.3]heptan-2-yl)methanone;
(S)-2-(4-(8-((3-chloro-4-(6-prolyl-2,6-diazaspiro[3.3]heptane-2-carbonyl)phenyl)amino)imidazo[1,2-a]pyrazin-3-yl)-3-(trifluoromethyl)-1H-pyrazol-1-yl)acetonitrile;
(2S,4R)—N-(2-(4-((3-(1-(cyanomethyl)-3-(trifluoromethyl)-1H-pyrazol-4-yl)imidazo[1,2-a]pyrazin-8-yl)amino)-2-ethylbenzamido)ethyl)-4-hydroxypyrrolidine-2-carboxamide;
4-[[3-[1-(2,2-difluoroethyl)-3-(trifluoromethyl)pyrazol-4-yl]imidazo[1,2-a]pyrazin-8-yl]amino]-2-ethyl-N-[2-oxo-2-[[(3S)-pyrrolidin-3-yl]amino]ethyl]benzamide;
N-(2-(3-aminopropoxy)ethyl)-4-((3-(1-(1-cyanoethyl)-3-(trifluoromethyl)-1H-pyrazol-4-yl)imidazo[1,2-a]pyrazin-8-yl)amino)-2-ethylbenzamide;
N-((2-aminocyclopropyl)methyl)-2-ethyl-4-((3-(1-(2-fluoroethyl)-3-(trifluoromethyl)-1H-pyrazol-4-yl)imidazo[1,2-a]pyrazin-8-yl)amino)benzamide;
N-((2-aminocyclopropyl)methyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-[2-[(3-aminocyclobutyl)amino]-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
(R)—N-(2-(2-aminopropanamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-(2-aminoethoxy)ethyl)-2-ethyl-4-((3-(1-(3,3,3-trifluoro-2-hydroxypropyl)-3-(trifluoromethyl)-1H-pyrazol-4-yl)imidazo[1,2-a]pyrazin-8-yl)amino)benzamide;
4-((3-(1-(1-cyanoethyl)-3-(trifluoromethyl)-1H-pyrazol-4-yl)imidazo[1,2-a]pyrazin-8-yl)amino)-2-ethyl-N-(2-(2-(pyrrolidin-1-yl)ethoxy)ethyl)benzamide;
(4-(2-fluoro-6-methyl-4-((3-(1-(prop-2-yn-1-yl)-3-(trifluoromethyl)-1H-pyrazol-4-yl)imidazo[1,2-a]pyrazin-8-yl)amino)benzoyl)piperazin-1-yl)((2R,5S)-5-hydroxypyrrolidin-2-yl)methanone;
2-chloro-4-[[3-[1-(cyanomethyl)-3-(trifluoromethyl)pyrazol-4-yl]imidazo[1,2-a]pyrazin-8-yl]amino]-N-[(1R)-1-methyl-2-oxo-2-piperazin-1-yl-ethyl]benzamide;
4-[[3-[1-(2,2-difluoroethyl)-3-(trifluoromethyl)pyrazol-4-yl]imidazo[1,2-a]pyrazin-8-yl]amino]-2-ethyl-N-[2-oxo-2-[[(3R)-pyrrolidin-3-yl]amino]ethyl]benzamide;
(S)—N—((R)-2-(4-((3-(1-(2,2-difluoroethyl)-3-(trifluoromethyl)-1H-pyrazol-4-yl)imidazo[1,2-a]pyrazin-8-yl)amino)-2-ethylbenzamido)propyl)pyrrolidine-2-carboxamide;
2-amino-1-(4-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzoyl)piperazin-1-yl)ethan-1-one;
2-amino-1-(6-(2-chloro-4-((3-(1-(2,2-difluoroethyl)-3-(trifluoromethyl)-1H-pyrazol-4-yl)imidazo[1,2-a]pyrazin-8-yl)amino)benzoyl)-2,6-diazaspiro[3.3]heptan-2-yl)ethan-1-one;
(S)—N-(3-(2-aminopropanamido)propyl)-2-ethyl-4-((3-(3-(trifluoromethyl)-1H-pyrazol-4-yl)imidazo[1,2-a]pyrazin-8-yl)amino)benzamide;
(1R,5S,6r)-N—((R)-2-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamido)propyl)-3-azabicyclo[3.1.0]hexane-6-carboxamide;
(S)—N—((R)-2-(4-((3-(1-(cyanomethyl)-3-(trifluoromethyl)-1H-pyrazol-4-yl)imidazo[1,2-a]pyrazin-8-yl)amino)-2-ethylbenzamido)propyl)pyrrolidine-2-carboxamide;
N-(2-(2-aminoethoxy)ethyl)-4-((3-(1-(3-chloro-3-methylbutyl)-3-(trifluoromethyl)-1H-pyrazol-4-yl)imidazo[1,2-a]pyrazin-8-yl)amino)-2-ethylbenzamide;
N-[2-[(3S)-3-aminopyrrolidin-1-yl]-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
2-amino-1-(6-(2-chloro-4-((3-(3-(trifluoromethyl)-1H-pyrazol-4-yl)imidazo[1,2-a]pyrazin-8-yl)amino)benzoyl)-2,6-diazaspiro[3.3]heptan-2-yl)ethan-1-one;
2-(4-(8-((3-chloro-4-(6-glycyl-2,6-diazaspiro[3.3]heptane-2-carbonyl)phenyl)amino)imidazo[1,2-a]pyrazin-3-yl)-3-(trifluoromethyl)-1H-pyrazol-1-yl)acetonitrile;
N-[2-[(3-aminocyclobutyl)amino]-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
4-((3-(1-cyclobutyl-3-(trifluoromethyl)-1H-pyrazol-4-yl)imidazo[1,2-a]pyrazin-8-yl)amino)-2-ethyl-N-(2-(2-morpholinoethoxy)ethyl)benzamide;
(R)—N-(1-aminopropan-2-yl)-2-fluoro-6-methyl-4-((3-(1-(prop-2-yn-1-yl)-3-(trifluoromethyl)-1H-pyrazol-4-yl)imidazo[1,2-a]pyrazin-8-yl)amino)benzamide;
N-[2-[(3R)-3-aminopyrrolidin-1-yl]-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
(S)-2-amino-1-(4-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzoyl)piperazin-1-yl)propan-1-one;
N-[(1R)-2-[(3S)-3-aminopyrrolidin-1-yl]-1-methyl-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
N-[2-(4-amino-1-piperidyl)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
(2S,4R)—N-(2-(4-((3-(1-(2,2-difluoroethyl)-3-(trifluoromethyl)-1H-pyrazol-4-yl)imidazo[1,2-a]pyrazin-8-yl)amino)-2-ethylbenzamido)ethyl)-4-hydroxypyrrolidine-2-carboxamide;
4-[[3-[1-(2,2-difluoroethyl)-3-(trifluoromethyl)pyrazol-4-yl]imidazo[1,2-a]pyrazin-8-yl]amino]-N-[2-[2-(dimethylamino)ethylamino]-2-oxo-ethyl]-2-ethyl-benzamide;
N—((R)-1-((1s,3S)-3-aminocyclobutane-1-carboxamido)propan-2-yl)-4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamide;
N-(2-(3-aminopropoxy)ethyl)-4-((3-(1-(3,3-difluorocyclobutyl)-3-(trifluoromethyl)-1H-pyrazol-4-yl)imidazo[1,2-a]pyrazin-8-yl)amino)-2-ethylbenzamide;
N-((2-aminocyclopropyl)methyl)-4-((3-(1-cyclobutyl-3-(trifluoromethyl)-1H-pyrazol-4-yl)imidazo[1,2-a]pyrazin-8-yl)amino)-2-ethylbenzamide;
2-ethyl-N-(2-(2-(pyrrolidin-1-yl)ethoxy)ethyl)-4-((3-(3-(trifluoromethyl)-1H-pyrazol-4-yl)imidazo[1,2-a]pyrazin-8-yl)amino)benzamide;
(R)—N-(1-(2-aminoacetamido)propan-2-yl)-4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamide;
N-[(1R)-2-[(3R)-3-aminopyrrolidin-1-yl]-1-methyl-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
N-[2-[(3-aminocyclobutyl)amino]-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
2-(4-(8-((4-(4-((1R,5S,6r)-3-azabicyclo[3.1.0]hexane-6-carbonyl)piperazine-1-carbonyl)-3-fluoro-5-methylphenyl)amino)imidazo[1,2-a]pyrazin-3-yl)-3-(trifluoromethyl)-1H-pyrazol-1-yl)acetonitrile;
(R)—N-(3-(2-aminopropanamido)propyl)-2-ethyl-4-((3-(3-(trifluoromethyl)-1H-pyrazol-4-yl)imidazo[1,2-a]pyrazin-8-yl)amino)benzamide;
4-[[3-[1-(cyanomethyl)-3-(trifluoromethyl)pyrazol-4-yl]imidazo[1,2-a]pyrazin-8-yl]amino]-2-ethyl-N-[(1R)-1-methyl-2-oxo-2-piperazin-1-yl-ethyl]benzamide;
4-[[3-[1-(2,2-difluoroethyl)-3-(trifluoromethyl)pyrazol-4-yl]imidazo[1,2-a]pyrazin-8-yl]amino]-2-ethyl-N-(2-oxo-2-piperazin-1-yl-ethyl)benzamide;
(R)-4-((3-(1-allyl-3-(trifluoromethyl)-1H-pyrazol-4-yl)imidazo[1,2-a]pyrazin-8-yl)amino)-N-(1-aminopropan-2-yl)-2-fluoro-6-methylbenzamide;
N-[2-[(3S)-3-aminopyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
2-(4-(8-((4-(4-(L-alanyl)piperazine-1-carbonyl)-3-fluoro-5-methylphenyl)amino)imidazo[1,2-a]pyrazin-3-yl)-3-(trifluoromethyl)-1H-pyrazol-1-yl)acetonitrile;
N-[(1R)-2-[(3-aminocyclobutyl)amino]-1-methyl-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
(2S,4R)—N—((R)-2-(4-((3-(1-(2,2-difluoroethyl)-3-(trifluoromethyl)-1H-pyrazol-4-yl)imidazo[1,2-a]pyrazin-8-yl)amino)-2-ethylbenzamido)propyl)-4-hydroxypyrrolidine-2-carboxamide;
(R)-3-amino-N-(3-(4-((3-(1-(cyanomethyl)-3-(trifluoromethyl)-1H-pyrazol-4-yl)imidazo[1,2-a]pyrazin-8-yl)amino)-2-ethylbenzamido)propyl)pyrrolidine-1-carboxamide;
N-[2-[(3R)-3-aminopyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
N-[2-[(3R)-3-aminopyrrolidin-1-yl]-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
(S)-(2-chloro-4-((3-(3-(trifluoromethyl)-1H-pyrazol-4-yl)imidazo[1,2-a]pyrazin-8-yl)amino)phenyl)(6-prolyl-2,6-diazaspiro[3.3]heptan-2-yl)methanone;
(R)—N-(1-(2-aminoacetamido)propan-2-yl)-4-((3-(1-(cyanomethyl)-3-(trifluoromethyl)-1H-pyrazol-4-yl)imidazo[1,2-a]pyrazin-8-yl)amino)-2-fluoro-6-methylbenzamide;
N-[(1R)-2-(4-amino-1-piperidyl)-1-methyl-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
N-(2-(2-aminoacetamido)ethyl)-2-ethyl-4-((3-(3-(trifluoromethyl)-1H-pyrazol-4-yl)imidazo[1,2-a]pyrazin-8-yl)amino)benzamide;
(4-((1R,5S,6r)-3-azabicyclo[3.1.0]hexane-6-carbonyl)piperazin-1-yl)(4-((3-(1-(2,2-difluoroethyl)-3-(trifluoromethyl)-1H-pyrazol-4-yl)imidazo[1,2-a]pyrazin-8-yl)amino)-2-fluoro-6-methylphenyl)methanone;
2-chloro-4-[[3-[1-(cyanomethyl)-3-(trifluoromethyl)pyrazol-4-yl]imidazo[1,2-a]pyrazin-8-yl]amino]-N-[2-[2-(dimethylamino)ethylamino]-2-oxo-ethyl]benzamide;
(S)-2-amino-1-(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)propan-1-one;
2-(4-(8-((3-fluoro-4-(4-glycylpiperazine-1-carbonyl)-5-methylphenyl)amino)imidazo[1,2-a]pyrazin-3-yl)-3-(trifluoromethyl)-1H-pyrazol-1-yl)acetonitrile;
(R)—N-(2-(2-aminopropanamido)ethyl)-2-ethyl-4-((3-(3-(trifluoromethyl)-1H-pyrazol-4-yl)imidazo[1,2-a]pyrazin-8-yl)amino)benzamide;
N-[(1R)-2-(4-amino-1-piperidyl)-1-methyl-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
(4-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzoyl)piperazin-1-yl)((2S,4R)-4-hydroxypyrrolidin-2-yl)methanone;
2-(4-(8-((4-(4-((1s,3s)-3-aminocyclobutane-1-carbonyl)piperazine-1-carbonyl)-3-fluoro-5-methylphenyl)amino)imidazo[1,2-a]pyrazin-3-yl)-3-(trifluoromethyl)-1H-pyrazol-1-yl)acetonitrile;
N-((2-aminocyclopropyl)methyl)-4-((3-(1-(1-cyanoethyl)-3-(trifluoromethyl)-1H-pyrazol-4-yl)imidazo[1,2-a]pyrazin-8-yl)amino)-2-ethylbenzamide;
(4-((1s,3s)-3-aminocyclobutane-1-carbonyl)piperazin-1-yl)(4-((3-(1-(2,2-difluoroethyl)-3-(trifluoromethyl)-1H-pyrazol-4-yl)imidazo[1,2-a]pyrazin-8-yl)amino)-2-fluoro-6-methylphenyl)methanone;
N-(3-(3-aminopropanamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
2-(4-(8-((3-fluoro-5-methyl-4-(4-(piperidine-4-carbonyl)piperazine-1-carbonyl)phenyl)amino)imidazo[1,2-a]pyrazin-3-yl)-3-(trifluoromethyl)-1H-pyrazol-1-yl)acetonitrile;
(R)—N-(1-aminopropan-2-yl)-4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamide;
(R)—N-(2-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamido)propyl)piperidine-4-carboxamide;
4-[[3-[1-(cyanomethyl)-3-(trifluoromethyl)pyrazol-4-yl]imidazo[1,2-a]pyrazin-8-yl]amino]-2-methyl-N-(2-oxo-2-piperazin-1-yl-ethyl)benzamide;
(4-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzoyl)piperazin-1-yl)(piperidin-4-yl)methanone;
2-ethyl-N-(3-(2-(2,2,2-trifluoroacetamido)acetamido)propyl)-4-((3-(3-(trifluoromethyl)-1H-pyrazol-4-yl)imidazo[1,2-a]pyrazin-8-yl)amino)benzamide;
N—((R)-1-((1s,3S)-3-aminocyclobutane-1-carboxamido)propan-2-yl)-4-((3-(1-(cyanomethyl)-3-(trifluoromethyl)-1H-pyrazol-4-yl)imidazo[1,2-a]pyrazin-8-yl)amino)-2-fluoro-6-methylbenzamide;
2-chloro-4-[[3-[1-(cyanomethyl)-3-(trifluoromethyl)pyrazol-4-yl]imidazo[1,2-a]pyrazin-8-yl]amino]-N-[(1R)-1-methyl-2-oxo-2-[[(3S)-pyrrolidin-3-yl]amino]ethyl]benzamide;
N-[2-[(3S)-3-aminopyrrolidin-1-yl]-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
(2S,4R)—N—((R)-2-(4-((3-(1-(cyanomethyl)-3-(trifluoromethyl)-1H-pyrazol-4-yl)imidazo[1,2-a]pyrazin-8-yl)amino)-2-ethylbenzamido)propyl)-4-hydroxypyrrolidine-2-carboxamide;
N-[2-(3-aminoazetidin-1-yl)-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
(4-(2-fluoro-4-((3-(1-(2-fluoroethyl)-3-(trifluoromethyl)-1H-pyrazol-4-yl)imidazo[1,2-a]pyrazin-8-yl)amino)-6-methylbenzoyl)piperazin-1-yl)((2R,5S)-5-hydroxypyrrolidin-2-yl)methanone;
2-(4-(8-((3-fluoro-4-(4-((2R,5S)-5-hydroxypyrrolidine-2-carbonyl)piperazine-1-carbonyl)-5-methylphenyl)amino)imidazo[1,2-a]pyrazin-3-yl)-3-(trifluoromethyl)-1H-pyrazol-1-yl)acetonitrile;
N-[2-(3-aminoazetidin-1-yl)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
4-((3-(1-(2,2-difluoroethyl)-3-(trifluoromethyl)-1H-pyrazol-4-yl)imidazo[1,2-a]pyrazin-8-yl)amino)-2-ethyl-N-(2-(2-morpholinoethoxy)ethyl)benzamide;
(S)—N-(2-(2-aminopropanamido)ethyl)-2-ethyl-4-((3-(3-(trifluoromethyl)-1H-pyrazol-4-yl)imidazo[1,2-a]pyrazin-8-yl)amino)benzamide;
(R)—N-(1-aminopropan-2-yl)-4-((3-(1-(cyanomethyl)-3-(trifluoromethyl)-1H-pyrazol-4-yl)imidazo[1,2-a]pyrazin-8-yl)amino)-2-fluoro-6-methylbenzamide;
(1R,5S,6r)-N—((R)-2-(4-((3-(1-(cyanomethyl)-3-(trifluoromethyl)-1H-pyrazol-4-yl)imidazo[1,2-a]pyrazin-8-yl)amino)-2-fluoro-6-methylbenzamido)propyl)-3-azabicyclo[3.1.0]hexane-6-carboxamide;
2-(4-(8-((3-fluoro-4-(4-((2S,4R)-4-hydroxypyrrolidine-2-carbonyl)piperazine-1-carbonyl)-5-methylphenyl)amino)imidazo[1,2-a]pyrazin-3-yl)-3-(trifluoromethyl)-1H-pyrazol-1-yl)acetonitrile;
N—((R)-1-((S)-2-aminopropanamido)propan-2-yl)-4-((3-(1-(cyanomethyl)-3-(trifluoromethyl)-1H-pyrazol-4-yl)imidazo[1,2-a]pyrazin-8-yl)amino)-2-fluoro-6-methylbenzamide;
2-chloro-4-[[3-[1-(cyanomethyl)-3-(trifluoromethyl)pyrazol-4-yl]imidazo[1,2-a]pyrazin-8-yl]amino]-N-[2-oxo-2-[[(3R)-pyrrolidin-3-yl]amino]ethyl]benzamide;
N-(2-((1S,3R)-3-aminocyclopentane-1-carboxamido)ethyl)-2-ethyl-4-((3-(3-(trifluoromethyl)-1H-pyrazol-4-yl)imidazo[1,2-a]pyrazin-8-yl)amino)benzamide;
N-[(1R)-2-[(3-aminocyclobutyl)amino]-1-methyl-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
4-[[3-[1-(cyanomethyl)-3-(trifluoromethyl)pyrazol-4-yl]imidazo[1,2-a]pyrazin-8-yl]amino]-2-methyl-N-[(1R)-1-methyl-2-oxo-2-piperazin-1-yl-ethyl]benzamide;
(2S,4R)—N-(3-(4-((3-(1-(2,2-difluoroethyl)-3-(trifluoromethyl)-1H-pyrazol-4-yl)imidazo[1,2-a]pyrazin-8-yl)amino)-2-ethylbenzamido)propyl)-4-hydroxypyrrolidine-2-carboxamide;
N-[2-(2-aminoethylamino)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)(piperidin-4-yl)methanone;
2-chloro-4-[[3-[1-(cyanomethyl)-3-(trifluoromethyl)pyrazol-4-yl]imidazo[1,2-a]pyrazin-8-yl]amino]-N-[2-oxo-2-[[(3S)-pyrrolidin-3-yl]amino]ethyl]benzamide;
N-[(1R)-2-[(3R)-3-aminopyrrolidin-1-yl]-1-methyl-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
N-[2-(3-aminoazetidin-1-yl)-2-oxo-ethyl]-4-[[3-[1-(2,2-difluoroethyl)-3-(trifluoromethyl)pyrazol-4-yl]imidazo[1,2-a]pyrazin-8-yl]amino]-2-ethyl-benzamide;
(4-((1r,3r)-3-aminocyclobutane-1-carbonyl)piperazin-1-yl)(4-((3-(3-(difluoromethyl)-1-(prop-2-yn-1-yl)-1H-pyrazol-4-yl)imidazo[1,2-a]pyrazin-8-yl)amino)-2-ethylphenyl)methanone;
(R)—N-(1-aminopropan-2-yl)-2-fluoro-4-((3-(1-(2-fluoroethyl)-3-(trifluoromethyl)-1H-pyrazol-4-yl)imidazo[1,2-a]pyrazin-8-yl)amino)-6-methylbenzamide;
N-[(1R)-2-[(3S)-3-aminopyrrolidin-1-yl]-1-methyl-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
2-ethyl-4-((3-(1-(2-fluoroethyl)-3-(trifluoromethyl)-1H-pyrazol-4-yl)imidazo[1,2-a]pyrazin-8-yl)amino)-N-(2-(2-morpholinoethoxy)ethyl)benzamide;
4-[[3-[1-(cyanomethyl)-3-(trifluoromethyl)pyrazol-4-yl]imidazo[1,2-a]pyrazin-8-yl]amino]-2-methyl-N-[(1R)-1-methyl-2-oxo-2-[[(3S)-pyrrolidin-3-yl]amino]ethyl]benzamide;
N-[2-[(3S)-3-aminopyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
N-(2-((1s,3s)-3-aminocyclobutane-1-carboxamido)ethyl)-2-ethyl-4-((3-(3-(trifluoromethyl)-1H-pyrazol-4-yl)imidazo[1,2-a]pyrazin-8-yl)amino)benzamide;
(R)—N-(2-(4-((3-(1-(cyanomethyl)-3-(trifluoromethyl)-1H-pyrazol-4-yl)imidazo[1,2-a]pyrazin-8-yl)amino)-2-fluoro-6-methylbenzamido)propyl)piperidine-4-carboxamide;
(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)(piperidin-4-yl)methanone;
(4-((1r,3r)-3-aminocyclobutane-1-carbonyl)piperazin-1-yl)(4-((3-(1-(2,2-difluoroethyl)-3-(difluoromethyl)-1H-pyrazol-4-yl)imidazo[1,2-a]pyrazin-8-yl)amino)-2-ethylphenyl)methanone;
(2S,4R)—N—((R)-2-(4-((3-(1-(2,2-difluoroethyl)-3-(trifluoromethyl)-1H-pyrazol-4-yl)imidazo[1,2-a]pyrazin-8-yl)amino)-2-fluoro-6-methylbenzamido)propyl)-4-hydroxypyrrolidine-2-carboxamide;
4-((3-(1-(1-cyanoethyl)-3-(trifluoromethyl)-1H-pyrazol-4-yl)imidazo[1,2-a]pyrazin-8-yl)amino)-2-ethyl-N-(2-(2-morpholinoethoxy)ethyl)benzamide;
4-[[3-[1-(cyanomethyl)-3-(trifluoromethyl)pyrazol-4-yl]imidazo[1,2-a]pyrazin-8-yl]amino]-2-methyl-N-[(1R)-1-methyl-2-oxo-2-[[(3R)-pyrrolidin-3-yl]amino]ethyl]benzamide;
(R)-2-amino-1-(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)propan-1-one;
(R)-2-ethyl-N-(3-(2-(2,2,2-trifluoroacetamido)propanamido)propyl)-4-((3-(3-(trifluoromethyl)-1H-pyrazol-4-yl)imidazo[1,2-a]pyrazin-8-yl)amino)benzamide;
N-[2-[(3-aminocyclobutyl)amino]-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
4-[[3-[1-(cyanomethyl)-3-(trifluoromethyl)pyrazol-4-yl]imidazo[1,2-a]pyrazin-8-yl]amino]-2-methyl-N-[2-oxo-2-[[(3R)-pyrrolidin-3-yl]amino]ethyl]benzamide;
N-[2-(3-aminoazetidin-1-yl)-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
4-[[3-[1-(cyanomethyl)-3-(trifluoromethyl)pyrazol-4-yl]imidazo[1,2-a]pyrazin-8-yl]amino]-N-[2-[2-(dimethylamino)ethylamino]-2-oxo-ethyl]-2-methyl-benzamide;
2-ethyl-N-(2-oxo-2-piperazin-1-yl-ethyl)-4-[[3-[3-(trifluoromethyl)-1H-pyrazol-4-yl]imidazo[1,2-a]pyrazin-8-yl]amino]benzamide;
(2S,4R)—N-(3-(4-((3-(1-(cyanomethyl)-3-(trifluoromethyl)-1H-pyrazol-4-yl)imidazo[1,2-a]pyrazin-8-yl)amino)-2-ethylbenzamido)propyl)-4-hydroxypyrrolidine-2-carboxamide;
(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)(4-hydroxypiperidin-4-yl)methanone;
N-[2-(4-amino-1-piperidyl)-2-oxo-ethyl]-2-ethyl-4-[[3-[3-(trifluoromethyl)-1H-pyrazol-4-yl]imidazo[1,2-a]pyrazin-8-yl]amino]benzamide;
4-[[3-[1-(cyanomethyl)-3-(trifluoromethyl)pyrazol-4-yl]imidazo[1,2-a]pyrazin-8-yl]amino]-2-ethyl-N-[2-oxo-2-[[(3R)-pyrrolidin-3-yl]amino]ethyl]benzamide;
4-[[3-[1-(cyanomethyl)-3-(trifluoromethyl)pyrazol-4-yl]imidazo[1,2-a]pyrazin-8-yl]amino]-2-ethyl-N-[2-oxo-2-[[(3S)-pyrrolidin-3-yl]amino]ethyl]benzamide;
(S)-2-ethyl-N-(3-(2-(2,2,2-trifluoroacetamido)propanamido)propyl)-4-((3-(3-(trifluoromethyl)-1H-pyrazol-4-yl)imidazo[1,2-a]pyrazin-8-yl)amino)benzamide;
N-[2-(2-aminoethylamino)-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
N-[(1R)-2-[(3R)-3-aminopyrrolidin-1-yl]-1-methyl-2-oxo-ethyl]-4-[[3-[1-(cyanomethyl)-3-(trifluoromethyl)pyrazol-4-yl]imidazo[1,2-a]pyrazin-8-yl]amino]-2-methyl-benzamide;
(S)—N—((R)-2-(2-ethyl-4-((3-(3-(trifluoromethyl)-1H-pyrazol-4-yl)imidazo[1,2-a]pyrazin-8-yl)amino)benzamido)propyl)pyrrolidine-2-carboxamide;
(S)-(2-chloro-4-((3-(1-(difluoromethyl)-3-(trifluoromethyl)-1H-pyrazol-4-yl)imidazo[1,2-a]pyrazin-8-yl)amino)phenyl)(6-prolyl-2,6-diazaspiro[3.3]heptan-2-yl)methanone;
(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)(4-hydroxypiperidin-4-yl)methanone;
N-[2-(2-aminoethylamino)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
2-amino-1-(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)ethan-1-one;
(S)-2-amino-1-(4-(4-((3-(3-(difluoromethyl)-1-(methoxymethyl)-1H-pyrazol-4-yl)imidazo[1,2-a]pyrazin-8-yl)amino)-2-ethylbenzoyl)piperazin-1-yl)propan-1-one;
(R)-2-amino-1-(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)-3-methylbutan-1-one;
(2S,4R)—N—((R)-2-(4-((3-(1-(cyanomethyl)-3-(trifluoromethyl)-1H-pyrazol-4-yl)imidazo[1,2-a]pyrazin-8-yl)amino)-2-fluoro-6-methylbenzamido)propyl)-4-hydroxypyrrolidine-2-carboxamide;
(4-(4-((3-(3-(difluoromethyl)-1-(2-fluoroethyl)-1H-pyrazol-4-yl)imidazo[1,2-a]pyrazin-8-yl)amino)-2-ethylbenzoyl)piperazin-1-yl)(piperidin-4-yl)methanone;
2-amino-1-(4-(4-((3-(3-(difluoromethyl)-1-(methoxymethyl)-1H-pyrazol-4-yl)imidazo[1,2-a]pyrazin-8-yl)amino)-2-ethylbenzoyl)piperazin-1-yl)ethan-1-one;
2-ethyl-N-(2-(2-morpholinoethoxy)ethyl)-4-((3-(3-(trifluoromethyl)-1H-pyrazol-4-yl)imidazo[1,2-a]pyrazin-8-yl)amino)benzamide;
(R)-2-amino-1-(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)-3-methylbutan-1-one;
(4-(4-((3-(1-allyl-3-(difluoromethyl)-1H-pyrazol-4-yl)imidazo[1,2-a]pyrazin-8-yl)amino)-2-ethylbenzoyl)piperazin-1-yl)((2S,4R)-4-hydroxypyrrolidin-2-yl)methanone;
(S)-2-amino-1-(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)propan-1-one;
N-((2-aminocyclopropyl)methyl)-2-ethyl-4-((3-(3-(trifluoromethyl)-1H-pyrazol-4-yl)imidazo[1,2-a]pyrazin-8-yl)amino)benzamide;
(R)-2-amino-1-(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)propan-1-one;
(R)-2-amino-1-(4-(4-((3-(3-(difluoromethyl)-1-(methoxymethyl)-1H-pyrazol-4-yl)imidazo[1,2-a]pyrazin-8-yl)amino)-2-ethylbenzoyl)piperazin-1-yl)propan-1-one;
(S)-2-amino-1-(4-(4-((3-(3-(difluoromethyl)-1-(2-fluoroethyl)-1H-pyrazol-4-yl)imidazo[1,2-a]pyrazin-8-yl)amino)-2-ethylbenzoyl)piperazin-1-yl)propan-1-one;
4-[[3-[1-(cyanomethyl)-3-(trifluoromethyl)pyrazol-4-yl]imidazo[1,2-a]pyrazin-8-yl]amino]-2-methyl-N-[2-oxo-2-[[(3S)-pyrrolidin-3-yl]amino]ethyl]benzamide;
N-[2-(2-aminoethoxy)ethyl]-4-[[3-[1-(cyanomethyl)-3-phenyl-pyrazol-4-yl]imidazo[1,2-a]pyrazin-8-yl]amino]-2-ethyl-benzamide;
(2S,4R)—N—((R)-2-(2-ethyl-4-((3-(3-(trifluoromethyl)-1H-pyrazol-4-yl)imidazo[1,2-a]pyrazin-8-yl)amino)benzamido)propyl)-4-hydroxypyrrolidine-2-carboxamide;
2-fluoro-6-methyl-N-(4-(prop-2-yn-1-ylamino)butyl)-4-((3-(3-(trifluoromethyl)-1H-pyrazol-4-yl)imidazo[1,2-a]pyrazin-8-yl)amino)benzamide;
(2R)-2-amino-1-(4-(4-((3-(1-(but-3-yn-2-yl)-3-(difluoromethyl)-1H-pyrazol-4-yl)imidazo[1,2-a]pyrazin-8-yl)amino)-2-ethylbenzoyl)piperazin-1-yl)propan-1-one;
(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)((2S,4R)-4-hydroxypyrrolidin-2-yl)methanone;
(4-(4-((3-(1-(2,2-difluoroethyl)-3-(difluoromethyl)-1H-pyrazol-4-yl)imidazo[1,2-a]pyrazin-8-yl)amino)-2-ethylbenzoyl)piperazin-1-yl)((2S,4R)-4-hydroxypyrrolidin-2-yl)methanone;
(4-(4-((3-(1-(but-3-yn-2-yl)-3-(difluoromethyl)-1H-pyrazol-4-yl)imidazo[1,2-a]pyrazin-8-yl)amino)-2-ethylbenzoyl)piperazin-1-yl)((2R,4S)-4-hydroxypyrrolidin-2-yl)methanone;
2-amino-1-(4-(4-((3-(3-(difluoromethyl)-1-(2-fluoroethyl)-1H-pyrazol-4-yl)imidazo[1,2-a]pyrazin-8-yl)amino)-2-ethylbenzoyl)piperazin-1-yl)ethan-1-one;
(S)—N-(3-(4-((3-(1-(2-amino-2-oxoethyl)-3-(trifluoromethyl)-1H-pyrazol-4-yl)imidazo[1,2-a]pyrazin-8-yl)amino)-2-ethylbenzamido)propyl)pyrrolidine-2-carboxamide;
N-[(1R)-2-[(3-aminocyclobutyl)amino]-1-methyl-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
(4-(4-((3-(3-(difluoromethyl)-1-(2-fluoroethyl)-1H-pyrazol-4-yl)imidazo[1,2-a]pyrazin-8-yl)amino)-2-ethylbenzoyl)piperazin-1-yl)(4-hydroxypiperidin-4-yl)methanone;
2-(3-(difluoromethyl)-4-(8-((3-ethyl-4-(4-(piperidine-4-carbonyl)piperazine-1-carbonyl)phenyl)amino)imidazo[1,2-a]pyrazin-3-yl)-1H-pyrazol-1-yl)acetonitrile;
2-amino-1-(4-(4-((3-(3-(difluoromethyl)-1-(prop-2-yn-1-yl)-1H-pyrazol-4-yl)imidazo[1,2-a]pyrazin-8-yl)amino)-2-ethylbenzoyl)piperazin-1-yl)ethan-1-one;
(S)—N-(3-(2-aminopropanamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
(4-(4-((3-(3-(difluoromethyl)-1-(2-fluoroethyl)-1H-pyrazol-4-yl)imidazo[1,2-a]pyrazin-8-yl)amino)-2-ethylbenzoyl)piperazin-1-yl)((2S,4R)-4-hydroxypyrrolidin-2-yl)methanone;
(R)-2-amino-1-(4-(4-((3-(3-(difluoromethyl)-1-(2-fluoroethyl)-1H-pyrazol-4-yl)imidazo[1,2-a]pyrazin-8-yl)amino)-2-ethylbenzoyl)piperazin-1-yl)propan-1-one;
2-(4-(8-((4-(4-(D-valyl)piperazine-1-carbonyl)-3-ethylphenyl)amino)imidazo[1,2-a]pyrazin-3-yl)-3-(difluoromethyl)-1H-pyrazol-1-yl)acetonitrile;
(4-(4-((3-(3-(difluoromethyl)-1-(methoxymethyl)-1H-pyrazol-4-yl)imidazo[1,2-a]pyrazin-8-yl)amino)-2-ethylbenzoyl)piperazin-1-yl)(4-hydroxypyrrolidin-2-yl)methanone;
(2R)-2-amino-1-(4-(4-((3-(1-(but-3-yn-2-yl)-3-(difluoromethyl)-1H-pyrazol-4-yl)imidazo[1,2-a]pyrazin-8-yl)amino)-2-ethylbenzoyl)piperazin-1-yl)-3-methylbutan-1-one;
2-(4-(8-((4-(4-((1r,3r)-3-aminocyclobutane-1-carbonyl)piperazine-1-carbonyl)-3-ethylphenyl)amino)imidazo[1,2-a]pyrazin-3-yl)-3-(difluoromethyl)-1H-pyrazol-1-yl)acetonitrile;
(4-((3-(3-(difluoromethyl)-1-(methoxymethyl)-1H-pyrazol-4-yl)imidazo[1,2-a]pyrazin-8-yl)amino)-2-ethylphenyl)(piperazin-1-yl)methanone;
N-(3-(2-aminoacetamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
4-[[3-[1-(cyanomethyl)-3-(trifluoromethyl)pyrazol-4-yl]imidazo[1,2-a]pyrazin-8-yl]amino]-2-ethyl-N-[(1R)-1-methyl-2-oxo-2-[[(3R)-pyrrolidin-3-yl]amino]ethyl]benzamide;
N-(2-((1s,3s)-3-aminocyclobutane-1-carboxamido)ethyl)-2-ethyl-4-((3-(3-(trifluoromethyl)-1H-pyrazol-4-yl)imidazo[1,2-a]pyrazin-8-yl)amino)benzamide;
N-[3-[(3-aminocyclobutanecarbonyl)amino]propyl]-4-[[3-[1-(3-aminocyclobutanecarbonyl)-3-(trifluoromethyl)pyrazol-4-yl]imidazo[1,2-a]pyrazin-8-yl]amino]-2-ethyl-benzamide;
(2-fluoro-4-((3-(3-(trifluoromethyl)-1H-pyrazol-4-yl)imidazo[1,2-a]pyrazin-8-yl)amino)phenyl)(piperazin-1-yl)methanone;
(4-((1S,3R)-3-aminocyclopentane-1-carbonyl)piperazin-1-yl)(4-((3-(1-(2,2-difluoroethyl)-3-(trifluoromethyl)-1H-pyrazol-4-yl)imidazo[1,2-a]pyrazin-8-yl)amino)-2-fluorophenyl)methanone;
2-(4-(8-((4-(4-((1S,3R)-3-aminocyclopentane-1-carbonyl)piperazine-1-carbonyl)-3-fluorophenyl)amino)imidazo[1,2-a]pyrazin-3-yl)-3-(trifluoromethyl)-1H-pyrazol-1-yl)acetonitrile;
(4-((1S,3R)-3-aminocyclopentane-1-carbonyl)piperazin-1-yl)(4-((3-(1-(cyclopropylmethyl)-3-(trifluoromethyl)-1H-pyrazol-4-yl)imidazo[1,2-a]pyrazin-8-yl)amino)-2-fluorophenyl)methanone;
N-(2-(2-aminoethoxy)ethyl)-4-((3-(1-(2,2-difluoroethyl)-3-(trifluoromethyl)-1H-pyrazol-4-yl)imidazo[1,2-a]pyrazin-8-yl)amino)-2-fluorobenzamide;
N-(2-(2-aminoethoxy)ethyl)-4-((3-(1-(cyanomethyl)-3-(trifluoromethyl)-1H-pyrazol-4-yl)imidazo[1,2-a]pyrazin-8-yl)amino)-2-fluorobenzamide;
N-(2-(2-aminoethoxy)ethyl)-4-((3-(1-(cyclopropylmethyl)-3-(trifluoromethyl)-1H-pyrazol-4-yl)imidazo[1,2-a]pyrazin-8-yl)amino)-2-fluorobenzamide;
2-amino-1-(4-(4-((3-(1-(2,2-difluoroethyl)-3-(trifluoromethyl)-1H-pyrazol-4-yl)imidazo[1,2-a]pyrazin-8-yl)amino)-2-fluorobenzoyl)piperazin-1-yl)ethan-1-one;
2-(4-(8-((3-fluoro-4-(4-glycylpiperazine-1-carbonyl)phenyl)amino)imidazo[1,2-a]pyrazin-3-yl)-3-(trifluoromethyl)-1H-pyrazol-1-yl)acetonitrile;
2-amino-1-(4-(4-((3-(1-(cyclopropylmethyl)-3-(trifluoromethyl)-1H-pyrazol-4-yl)imidazo[1,2-a]pyrazin-8-yl)amino)-2-fluorobenzoyl)piperazin-1-yl)ethan-1-one;
N-(2-((2-((2-aminoethyl)amino)-2-oxoethyl)amino)ethyl)-2-chloro-4-((3-(1-(cyanomethyl)-3-(trifluoromethyl)-1H-pyrazol-4-yl)imidazo[1,2-a]pyrazin-8-yl)amino)benzamide;
N-(2-((2-(azetidin-3-ylamino)-2-oxoethyl)amino)ethyl)-2-chloro-4-((3-(1-(cyanomethyl)-3-(trifluoromethyl)-1H-pyrazol-4-yl)imidazo[1,2-a]pyrazin-8-yl)amino)benzamide;
(S)-2-chloro-4-((3-(1-(cyanomethyl)-3-(trifluoromethyl)-1H-pyrazol-4-yl)imidazo[1,2-a]pyrazin-8-yl)amino)-N-(2-((2-oxo-2-(pyrrolidin-3-ylamino)ethyl)amino)ethyl)benzamide;
(R)-2-chloro-4-((3-(1-(cyanomethyl)-3-(trifluoromethyl)-1H-pyrazol-4-yl)imidazo[1,2-a]pyrazin-8-yl)amino)-N-(2-((2-oxo-2-(pyrrolidin-3-ylamino)ethyl)amino)ethyl)benzamide;
N-(2-((2-((3-aminopropyl)amino)-2-oxoethyl)amino)ethyl)-2-chloro-4-((3-(1-(cyanomethyl)-3-(trifluoromethyl)-1H-pyrazol-4-yl)imidazo[1,2-a]pyrazin-8-yl)amino)benzamide;
N-(2-((2-((2-aminoethyl)amino)-2-oxoethyl)amino)ethyl)-2-chloro-4-((3-(1-(2,2-difluoroethyl)-3-(trifluoromethyl)-1H-pyrazol-4-yl)imidazo[1,2-a]pyrazin-8-yl)amino)benzamide;
N-(2-((2-(azetidin-3-ylamino)-2-oxoethyl)amino)ethyl)-2-chloro-4-((3-(1-(2,2-difluoroethyl)-3-(trifluoromethyl)-1H-pyrazol-4-yl)imidazo[1,2-a]pyrazin-8-yl)amino)benzamide;
(S)-2-chloro-4-((3-(1-(2,2-difluoroethyl)-3-(trifluoromethyl)-1H-pyrazol-4-yl)imidazo[1,2-a]pyrazin-8-yl)amino)-N-(2-((2-oxo-2-(pyrrolidin-3-ylamino)ethyl)amino)ethyl)benzamide;
(R)-2-chloro-4-((3-(1-(2,2-difluoroethyl)-3-(trifluoromethyl)-1H-pyrazol-4-yl)imidazo[1,2-a]pyrazin-8-yl)amino)-N-(2-((2-oxo-2-(pyrrolidin-3-ylamino)ethyl)amino)ethyl)benzamide;
N-(2-((2-((3-aminopropyl)amino)-2-oxoethyl)amino)ethyl)-2-chloro-4-((3-(1-(2,2-difluoroethyl)-3-(trifluoromethyl)-1H-pyrazol-4-yl)imidazo[1,2-a]pyrazin-8-yl)amino)benzamide;
(1S,5R)-6-[[2-chloro-4-[[3-[1-(cyanomethyl)-3-(trifluoromethyl)pyrazol-4-yl]imidazo[1,2-a]pyrazin-8-yl]amino]benzoyl]amino]-N!-[(3R,4R)-4-hydroxypyrrolidin-3-yl]-3-azabicyclo[3.1.0]hexane-3-carboxamide;
N-(6-aminohexyl)-2-chloro-4-((3-(1-(2,2-difluoroethyl)-3-(trifluoromethyl)-1H-pyrazol-4-yl)imidazo[1,2-a]pyrazin-8-yl)amino)benzamide;
N-((1-(azetidin-3-ylmethyl)pyrrolidin-3-yl)methyl)-2-ethyl-4-((3-(3-(trifluoromethyl)-1H-pyrazol-4-yl)imidazo[1,2-a]pyrazin-8-yl)amino)benzamide;
N-((1-(azetidin-3-ylmethyl)piperidin-4-yl)methyl)-4-((3-(1-(cyanomethyl)-3-(trifluoromethyl)-1H-pyrazol-4-yl)imidazo[1,2-a]pyrazin-8-yl)amino)-2-ethylbenzamide;
2-[azetidin-3-ylmethyl-[5-[[4-[[3-[1-(2,2-difluoroethyl)-3-(trifluoromethyl)pyrazol-4-yl]imidazo[1,2-a]pyrazin-8-yl]amino]-2-ethyl-benzoyl]amino]pentyl]amino]acetic acid;
N-[2-(2-aminoethoxy)ethyl]-4-[[3-[3-cyano-1-(cyanomethyl)pyrazol-4-yl]imidazo[1,2-a]pyrazin-8-yl]amino]-2-ethyl-benzamide;
N-[2-(2-aminoethoxy)ethyl]-4-[[3-[3-cyano-1-(2,2-difluoroethyl)pyrazol-4-yl]imidazo[1,2-a]pyrazin-8-yl]amino]-2-ethyl-benzamide;
N-[2-(2-aminoethoxy)ethyl]-4-[[3-[1-(cyanomethyl)-3-methylsulfanyl-pyrazol-4-yl]imidazo[1,2-a]pyrazin-8-yl]amino]-2-ethyl-benzamide; and
N-[2-(2-aminoethoxy)ethyl]-4-[[3-[3-chloro-1-(cyanomethyl)pyrazol-4-yl]imidazo[1,2-a]pyrazin-8-yl]amino]-2-ethyl-benzamide.

In a preferred embodiment, the present invention provides a compound of formula (I) as described herein, or a pharmaceutically acceptable salt thereof, wherein the compound of formula (I) is selected from:
N-[2-(2-aminoethoxy)ethyl]-2-methyl-4-[[3-[1-prop-2-ynyl-3-(trifluoromethyl)pyrazol-4-yl]imidazo[1,2-a]pyrazin-8-yl]amino]benzamide;
N-[2-(2-aminoethoxy)ethyl]-4-[[3-[3-(difluoromethyl)-1-(methoxymethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(2-methoxyethyl)-3-(trifluoromethyl)pyrazol-4-yl]imidazo[1,2-a]pyrazin-8-yl]amino]benzamide;
N-[2-(2-aminoethoxy)ethyl]-4-[[3-[1-(2,2-difluoroethyl)-3-(trifluoromethyl)pyrazol-4-yl]imidazo[1,2-a]pyrazin-8-yl]amino]-2-fluoro-6-methylbenzamide;
N-[2-(2-amino-2-methylpropoxy)ethyl]-4-[[3-[1-(2,2-difluoroethyl)-3-(trifluoromethyl)pyrazol-4-yl]imidazo[1,2-a]pyrazin-8-yl]amino]-2-ethylbenzamide;
[2-chloro-4-[[3-[1-(difluoromethyl)-3-(trifluoromethyl)pyrazol-4-yl]imidazo[1,2-a]pyrazin-8-yl]amino]phenyl]-(2,6-diazaspiro[3.3]heptan-2-yl)methanone;
2-[4-[8-[3-chloro-4-(2,6-diazaspiro[3.3]heptane-2-carbonyl)anilino]imidazo[1,2-a]pyrazin-3-yl]-3-(trifluoromethyl)pyrazol-1-yl]acetonitrile;
[2-chloro-4-[[3-[1-(2,2-difluoroethyl)-3-(trifluoromethyl)pyrazol-4-yl]imidazo[1,2-a]pyrazin-8-yl]amino]phenyl]-[4-[(2S)-pyrrolidine-2-carbonyl]piperazin-1-yl]methanone;
N-[2-(2-aminoethylamino)-2-oxoethyl]-2-chloro-4-[[3-[1-prop-2-ynyl-3-(trifluoromethyl)pyrazol-4-yl]imidazo[1,2-a]pyrazin-8-yl]amino]benzamide;
N-[2-[(2R)-2-aminopropoxy]ethyl]-2-ethyl-4-[[3-[1-(2-fluoroethyl)-3-(trifluoromethyl)pyrazol-4-yl]imidazo[1,2-a]pyrazin-8-yl]amino]benzamide;
2-[4-[8-[4-[4-[(2S)-2-aminopropanoyl]piperazine-1-carbonyl]-3-chloroanilino]imidazo[1,2-a]pyrazin-3-yl]-3-(trifluoromethyl)pyrazol-1-yl]acetonitrile;
2-[4-[8-[4-[4-[(2R)-2-aminopropanoyl]piperazine-1-carbonyl]-3-chloroanilino]imidazo[1,2-a]pyrazin-3-yl]-3-(trifluoromethyl)pyrazol-1-yl]acetonitrile;
2-[4-[8-[4-[4-[(1S,3R)-3-aminocyclopentanecarbonyl]piperazine-1-carbonyl]-3-chloroanilino]imidazo[1,2-a]pyrazin-3-yl]-3-(trifluoromethyl)pyrazol-1-yl]acetonitrile;
[2-chloro-4-[[3-[3-(trifluoromethyl)-1H-pyrazol-4-yl]imidazo[1,2-a]pyrazin-8-yl]amino]phenyl]-[4-[(2S)-pyrrolidine-2-carbonyl]piperazin-1-yl]methanone;
2-[4-[8-[3-chloro-4-[4-[(2S)-pyrrolidine-2-carbonyl]piperazine-1-carbonyl]anilino]imidazo[1,2-a]pyrazin-3-yl]-3-(trifluoromethyl)pyrazol-1-yl]acetonitrile;
N-[2-(2-aminoethoxy)ethyl]-4-[[3-[1-cyclobutyl-3-(trifluoromethyl)pyrazol-4-yl]imidazo[1,2-a]pyrazin-8-yl]amino]-2-ethylbenzamide;
N-[2-(2-aminoethoxy)ethyl]-2-ethyl-4-[[3-[1-(2,2,2-trifluoroethyl)-3-(trifluoromethyl)pyrazol-4-yl]imidazo[1,2-a]pyrazin-8-yl]amino]benzamide;
[4-[(3R)-3-aminopyrrolidine-1-carbonyl]piperazin-1-yl]-[2-chloro-4-[[3-[3-(trifluoromethyl)-1H-pyrazol-4-yl]imidazo[1,2-a]pyrazin-8-yl]amino]phenyl]methanone;
N-[2-(2-aminoethoxy)ethyl]-4-[[3-[3-(difluoromethyl)-1-propylpyrazol-4-yl]imidazo[1,2-a]pyrazin-8-yl]amino]-2-ethylbenzamide;
4-[2-chloro-4-[[3-[3-(trifluoromethyl)-1H-pyrazol-4-yl]imidazo[1,2-a]pyrazin-8-yl]amino]benzoyl]-N-[(3S)-pyrrolidin-3-yl]piperazine-1-carboxamide;
[2-chloro-4-[[3-[3-(trifluoromethyl)-1H-pyrazol-4-yl]imidazo[1,2-a]pyrazin-8-yl]amino]phenyl]-[4-[(2S,4R)-4-hydroxypyrrolidine-2-carbonyl]piperazin-1-yl]methanone;
2-(4-(8-((4-(4-((1R,5S,6r)-3-azabicyclo[3.1.0]hexane-6-carbonyl)piperazine-1-carbonyl)-3-chlorophenyl)amino)imidazo[1,2-a]pyrazin-3-yl)-3-(trifluoromethyl)-1H-pyrazol-1-yl)acetonitrile;
N-(3-((1s,3s)-3-aminocyclobutane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1R,3S)-3-(12-azaneyl)cyclopentane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1S,3R)-3-aminocyclopentane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(3-((1S,3R)-3-aminocyclopentane-1-carboxamido)propyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-(4-((3-(1-(cyanomethyl)-3-(trifluoromethyl)-1H-pyrazol-4-yl)imidazo[1,2-a]pyrazin-8-yl)amino)-2-ethylbenzamido)ethyl)piperidine-4-carboxamide;
N-(2-((1r,3r)-3-aminocyclobutane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1r,3r)-3-aminocyclobutane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(S)-3-amino-N-(2-(4-((3-(1-(2,2-difluoroethyl)-3-(trifluoromethyl)-1H-pyrazol-4-yl)imidazo[1,2-a]pyrazin-8-yl)amino)-2-ethylbenzamido)ethyl)pyrrolidine-1-carboxamide;
N-(2-((1R,3R)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-((1S,3S)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1S,3S)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
(S)—N-(2-(2-aminopropanamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1R,3S)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1S,3R)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
N-(2-((1S,3R)-3-aminocyclopentane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
N-(2-((1R,3S)-3-aminocyclopentane-1-carboxamido)ethyl)-4-((3-(1-(2,2-difluoroethyl)-3-(trifluoromethyl)-1H-pyrazol-4-yl)imidazo[1,2-a]pyrazin-8-yl)amino)-2-ethylbenzamide;
cis-N-(2-((1s,3s)-3-aminocyclobutane-1-carboxamido)ethyl)-4-((3-(1-(cyanomethyl)-3-(trifluoromethyl)-1H-pyrazol-4-yl)imidazo[1,2-a]pyrazin-8-yl)amino)-2-ethylbenzamide;
4-[[3-[1-(cyanomethyl)-3-(trifluoromethyl)pyrazol-4-yl]imidazo[1,2-a]pyrazin-8-yl]amino]-2-ethyl-N-(2-oxo-2-piperazin-1-yl-ethyl)benzamide;
N-[2-(4-amino-1-piperidyl)-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide;
2-chloro-4-[[3-[1-(cyanomethyl)-3-(trifluoromethyl)pyrazol-4-yl]imidazo[1,2-a]pyrazin-8-yl]amino]-N-(2-oxo-2-piperazin-1-yl-ethyl)benzamide;
N-[2-(4-amino-1-piperidyl)-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide;
N-[2-(4-amino-1-piperidyl)-2-oxo-ethyl]-2-chloro-4-[[3-[1-(cyanomethyl)-3-(trifluoromethyl)pyrazol-4-yl]imidazo[1,2-a]pyrazin-8-yl]amino]benzamide; and
N-[2-[(3R)-3-aminopyrrolidin-1-yl]-2-oxo-ethyl]-4-[[3-[1-(cyanomethyl)-3-(trifluoromethyl)pyrazol-4-yl]imidazo[1,2-a]pyrazin-8-yl]amino]-2-ethyl-benzamide.

In one embodiment, the present invention provides a compound of formula (I) as described herein, or a pharmaceutically acceptable salt thereof, wherein:
R¹³ is selected from the group consisting of hydrogen, halogen, amino, C₁-C₆-alkyl, C₁-C₆-alkoxy, and C₃-C₁₂-cycloalkyl-C₁-C₆-alkyl-;
R¹⁴ is selected from the group consisting of hydrogen, halogen, and C₁-C₆-alkyl;
L¹ is selected from the group consisting of carbonyl, a covalent bond and C₁-C₆-alkyldiyl; and
A is selected from the group consisting of C₃-C₁₂-cycloalkyl, C₁-C₁₉-heterocyclyl, and C₁-C₁₃-heteroaryl.

In a preferred embodiment, the present invention provides a compound of formula (I) as described herein, or a pharmaceutically acceptable salt thereof, wherein:
R¹³ and R¹⁴ are both hydrogen;
L¹ is a covalent bond or —CH₂—; and
A is C₃-C₁₂-cycloalkyl.

In a particularly preferred embodiment, the present invention provides a compound of formula (I) as described herein, or a pharmaceutically acceptable salt thereof, wherein:
R¹³ and R¹⁴ are both hydrogen;
L¹ is a covalent bond or —CH₂—; and
A is cyclopropyl or cyclobutyl.

In one embodiment, the present invention provides a compound of formula (I) as described herein, or a pharmaceutically acceptable salt thereof, wherein:
R⁵ is halo-C₁-C₆-alkyl or a group

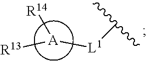

R¹³ is selected from the group consisting of hydrogen, halogen, amino, C₁-C₆-alkyl, C₁-C₆-alkoxy, and C₃-C₁₂-cycloalkyl-C₁-C₆-alkyl-;
R¹⁴ is selected from the group consisting of hydrogen, halogen, and C₁-C₆-alkyl;
L¹ is selected from the group consisting of carbonyl, a covalent bond and C₁-C₆-alkyldiyl; and
A is selected from the group consisting of C₃-C₁₂-cycloalkyl, C₁-C₁₉-heterocyclyl, and C₁-C₁₃-heteroaryl.

In one embodiment, the present invention provides a compound of formula (I) as described herein, or a pharmaceutically acceptable salt thereof, wherein:
R⁶ is selected from the group consisting of hydrogen, C₁-C₆-alkyl, C₂-C₆-alkenyl, C₂-C₆-alkynyl, halo-C₂-C₆-alkenyl, and a group

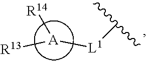

wherein the C₁-C₆-alkyl is optionally substituted with one to two substituents selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R¹³ is selected from the group consisting of hydrogen, halogen, amino, C₁-C₆-alkyl, C₁-C₆-alkoxy, and C₃-C₁₂-cycloalkyl-C₁-C₆-alkyl-;
R¹⁴ is selected from the group consisting of hydrogen, halogen, and C₁-C₆-alkyl;
L¹ is selected from the group consisting of carbonyl, a covalent bond and C₁-C₆-alkyldiyl; and
A is selected from the group consisting of C₃-C₁₂-cycloalkyl, C₁-C₁₉-heterocyclyl, and C₁-C₁₃-heteroaryl.

In a preferred embodiment, the present invention provides a compound of formula (I) as described herein, or a pharmaceutically acceptable salt thereof, wherein:
R⁶ is selected from the group consisting of hydrogen, C₁-C₆-alkyl, C₂-C₆-alkenyl, C₂-C₆-alkynyl, halo-C₂-C₆-alkenyl, and a group

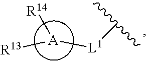

wherein the C₁-C₆-alkyl is optionally substituted with one to two substituents selected from the group consisting of C₁-C₆-alkoxy, halogen, cyano, and CF₃;
R¹³ and R¹⁴ are both hydrogen;
L¹ is a covalent bond or —CH₂—; and
A is C₃-C₁₂-cycloalkyl.

In a particularly preferred embodiment, the present invention provides a compound of formula (I) as described herein, or a pharmaceutically acceptable salt thereof, wherein:
R⁶ is selected from the group consisting of hydrogen, methyl, ethyl, propyl, vinyl, prop-2-ynyl, halo-C₂-C₆-alkenyl, and a group

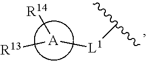

wherein said methyl, ethyl, and propyl are optionally substituted with one to two substituents selected from the group consisting of methoxy, fluoro, cyano, and CF₃;
R¹³ and R¹⁴ are both hydrogen;
L¹ is a covalent bond or —CH₂—; and
A is cyclopropyl or cyclobutyl.

In one embodiment, the present invention provides a compound of formula (I) as described herein, or a pharmaceutically acceptable salt thereof, wherein:
R¹⁵ is selected from the group consisting of hydrogen, amino, and hydroxy;
R¹⁶ is hydrogen or C₁-C₆-alkyl;
L³ is selected from the group consisting of a covalent bond, carbonyl, —NH—C(O)—, and —C(O)—NH—; and
B is C₁-C₁₉-heterocyclyl or C₃-C₁₂-cycloalkyl.

In a preferred embodiment, the present invention provides a compound of formula (I) as described herein, or a pharmaceutically acceptable salt thereof, wherein:
R¹⁵ is hydrogen or amino;
R¹⁶ is hydrogen;
L³ is carbonyl or —C(O)—NH—; and
B is C₁-C₁₉-heterocyclyl or C₃-C₁₂-cycloalkyl.

In a particularly preferred embodiment, the present invention provides a compound of formula (I) as described herein, or a pharmaceutically acceptable salt thereof, wherein:
R¹⁵ is hydrogen or amino;
R¹⁶ is hydrogen;
L³ is carbonyl or —C(O)—NH—; and
B is selected from the group consisting of pyrrolidin-1-yl, pyrrolidin-2-yl, cyclopentyl, cyclobutyl, piperazin-1-yl, and 1-piperidyl.

In one embodiment, the present invention provides a compound of formula (I) as described herein, or a pharmaceutically acceptable salt thereof, wherein:
R¹⁸ is selected from the group consisting of hydrogen, amino, and hydroxy;
R¹⁹ is hydrogen or C₁-C₆-alkyl;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—; and
C is C₁-C₁₉-heterocyclyl or C₃-C₁₂-cycloalkyl.

In a preferred embodiment, the present invention provides a compound of formula (I) as described herein, or a pharmaceutically acceptable salt thereof, wherein:
R¹⁸ is selected from the group consisting of hydrogen, amino, and hydroxy;
R¹⁹ is hydrogen;
L⁵ is carbonyl or —NH—C(O)—; and
C is C₁-C₁₉-heterocyclyl or C₃-C₁₂-cycloalkyl.

In a particularly preferred embodiment, the present invention provides a compound of formula (I) as described herein, or a pharmaceutically acceptable salt thereof, wherein:
R¹⁸ is selected from the group consisting of hydrogen, amino, and hydroxy;
R¹⁹ is hydrogen;
L⁵ is carbonyl or —NH—C(O)—; and
C is selected from the group consisting of pyrrolidin-1-yl, pyrrolidin-2-yl, pyrrolidin-3-yl, 1-piperidyl, 3-piperidyl, cyclobutyl, and cyclopentyl.

In one preferred embodiment, the present invention provides a compound of formula (I) as described herein, or a pharmaceutically acceptable salt thereof, wherein:
R¹¹ is hydroxy-C₁-C₁₉-heterocyclyl-C(O)—;
R¹² is selected from the group consisting of amino, and hydroxy-C₁-C₁₉-heterocyclyl-C(O)—NH—;
L⁶ is a covalent bond;
D is C₁-C₁₉-heterocyclyl or C₃-C₁₂-cycloalkyl.

In one embodiment, the present invention provides a compound of formula (I) as described herein, or a pharmaceutically acceptable salt thereof, wherein:
R¹⁰ is selected from the group consisting of amino, (C₁-C₆-alkyl)₂N—, C₂-C₆-alkynyl; amino-C₁-C₆-alkyl-NH—, carbamoyl, and guanidino; and
L² is selected from the group consisting of —C(O)—NH—, —NH—C(O)—, —NH—C(O)—NH—, —CH(NH₂)—C(O)—NH—, —NH—, and —O—.

In a preferred embodiment, the present invention provides a compound of formula (I) as described herein, or a pharmaceutically acceptable salt thereof, wherein:
R¹⁰ is amino; and
L² is —NH—C(O)— or —O—.

In one embodiment, the present invention provides a compound of formula (I) as described herein, or a pharmaceutically acceptable salt thereof, wherein:
R¹⁷ is selected from the group consisting of hydrogen, amino, and C₁-C₆-alkyl-NH—; and
L⁴ is selected from the group consisting of a covalent bond, carbonyl, —NH—C(O)—, —C(O)—NH—, and —NH—C(O)—CH₂—.

In a preferred embodiment, the present invention provides a compound of formula (I) as described herein, or a pharmaceutically acceptable salt thereof, wherein:
R¹⁷ is amino or C₁-C₆-alkyl-NH—; and
L⁴ is selected from the group consisting of a covalent bond, carbonyl, and —NH—C(O)—.

In a particularly preferred embodiment, the present invention provides a compound of formula (I) as described herein, or a pharmaceutically acceptable salt thereof, wherein:
R¹⁷ is amino or CH₃NH—; and
L⁴ is selected from the group consisting of a covalent bond, carbonyl, and —NH—C(O)—.

In one embodiment, the present invention provides a compound of formula (I) as described herein, or a pharmaceutically acceptable salt thereof, wherein:
(a) X is

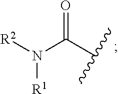

wherein:
   (i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, hydroxy, and a group

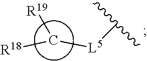

or
(ii) R¹ is selected from the group consisting of:
   (i) hydrogen;
   (ii) C₁-C₆-alkyl optionally substituted with one to two substituents selected from the group consisting of C₂-C₆-alkenyl-NH—, C₁-C₆-alkoxy, C₁-C₆-alkoxycarbonyl-NH—, C₂-C₆-alkynyl-NH—, amino, cyano, hydroxy, and a group

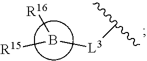

(iii) R¹⁰—C₁-C₆-alkyl-L²-C₁-C₆-alkyl-;
(iv) C₂-C₆-alkynyl;
(v) amino-C₁-C₆-alkoxy; and
(vi) a group

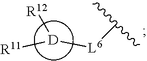

and
R² is hydrogen or C₁-C₆-alkyl; or
X is

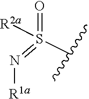

wherein R^{1a} is amino-C₁-C₆-alkyl and R² is C₁-C₆-alkyl;
R³ is selected from the group consisting of hydrogen, halogen, and C₁-C₆-alkyl;
R⁴ is hydrogen;
R⁷ is hydrogen or C₁-C₆-alkyl;
R⁸ is hydrogen;
R⁹ is selected from the group consisting of hydrogen, halogen, and C₁-C₆-alkyl;
R¹⁰ is selected from the group consisting of amino, (C₁-C₆-alkyl)₂N—, C₂-C₆-alkynyl; amino-C₁-C₆-alkyl-NH—, carbamoyl, and guanidino;
R¹¹ is hydroxy-C₁-C₁₉-heterocyclyl-C(O)—;
R¹² is selected from the group consisting of amino, and hydroxy-C₁-C₁₉-heterocyclyl-C(O)—NH—;
R¹⁵ is selected from the group consisting of hydrogen, amino, and hydroxy;
R¹⁶ is hydrogen or C₁-C₆-alkyl;
R¹⁷ is selected from the group consisting of hydrogen, amino, and C₁-C₆-alkyl-NH—;
R¹⁸ is selected from the group consisting of hydrogen, amino, and hydroxy;
R¹⁹ is hydrogen or C₁-C₆-alkyl;
L² is selected from the group consisting of —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is a covalent bond;
B is C₁-C₁₉-heterocyclyl or C₃-C₁₂-cycloalkyl;
C is C₁-C₁₉-heterocyclyl or C₃-C₁₂-cycloalkyl; and
D is C₁-C₁₉-heterocyclyl or C₃-C₁₂-cycloalkyl.

In a preferred embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, and a group

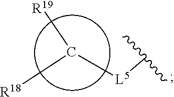

or
(ii) R¹ is selected from the group consisting of:
   (i) C₁-C₆-alkyl substituted with one substituent selected from a group

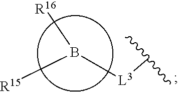

and
(ii) R¹⁰—C₁-C₆-alkyl-L²-C₁-C₆-alkyl-; and
R² is hydrogen;
R³ is C₁-C₆-alkyl or halogen;
R⁴ is hydrogen;
R⁷ is hydrogen;
R⁸ is hydrogen;
R⁹ is hydrogen or halogen;
R¹⁰ is amino;
R⁵ is hydrogen or amino;
R¹⁶ is hydrogen;
R¹⁷ is amino or C₁-C₆-alkyl-NH—;
R¹⁸ is selected from the group consisting of hydrogen, amino, and hydroxy;
R¹⁹ is hydrogen;
L² is —NH—C(O)— or —O—;
L³ is carbonyl or —C(O)—NH—;
L⁴ is selected from the group consisting of a covalent bond, carbonyl, and —NH—C(O)—;
L⁵ is carbonyl or —NH—C(O)—;
B is C₁-C₁₉-heterocyclyl or C₃-C₁₂-cycloalkyl; and
C is C₁-C₁₉-heterocyclyl or C₃-C₁₂-cycloalkyl.

In a particularly preferred embodiment, the present invention provides a compound of formula (II), (III) or (IV) as described herein, or a pharmaceutically acceptable salt thereof, wherein:
(i) R¹ and R², taken together with the N-atom to which they are attached, form a C₁-C₁₉-heterocyclic ring selected from the group consisting of 1-piperidyl, 2,6-diazaspiro[3.3]heptan-2-yl, azetidin-1-yl, and piperazin-1-yl, wherein the C₁-C₁₉-heterocyclic ring is optionally substituted with one to two substituents selected from the group consisting of R¹⁷—CH₂-L⁴-, R¹⁷—CH(CH₃)-L⁴-, R¹⁷—(CH₂)₃-L⁴-, amino, and a group

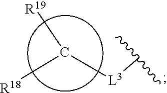

or
(ii) R¹ is selected from the group consisting of:
   (i) ethylydiyl (—(CH₂)₂—) substituted with one substituent selected from a group

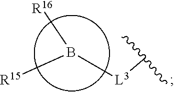

(ii) R¹⁰—(CH₂)₂-L¹-(CH₂)₂—;
(iii) R¹⁰—C(CH₃)₂—CH₂-L¹-(CH₂)₂—;
(iv) R¹⁰—(CH₂)₂-L¹-CH₂—; and
(v) R¹⁰—CH(CH₃)—CH₂-L¹-(CH₂)₂—; and
R² is hydrogen;
R³ is selected from the group consisting of methyl, ethyl, and chloro;
R⁴ is hydrogen;
R⁷ is hydrogen;
R⁸ is hydrogen;
R⁹ is hydrogen or fluoro;
R¹⁰ is amino;
R⁵ is hydrogen or amino;
R¹⁶ is hydrogen;
R¹⁷ is amino or CH₃NH—;
R¹⁸ is selected from the group consisting of hydrogen, amino, and hydroxy;
R¹⁹ is hydrogen;
L² is —NH—C(O)— or —O—;
L³ is carbonyl or —C(O)—NH—;
L⁴ is selected from the group consisting of a covalent bond, carbonyl, and —NH—C(O)—;
L⁵ is carbonyl or —NH—C(O)—;
B is selected from the group consisting of pyrrolidin-1-yl, pyrrolidin-2-yl, cyclopentyl, cyclobutyl, piperazin-1-yl, and 1-piperidyl; and
C is selected from the group consisting of pyrrolidin-1-yl, pyrrolidin-2-yl, pyrrolidin-3-yl, 1-piperidyl, 3-piperidyl, cyclobutyl, and cyclopentyl.

In one embodiment, the present invention provides pharmaceutically acceptable salts of the compounds of formula (I) as described herein, especially pharmaceutically acceptable salts selected from hydrochlorides, fumarates, formates, lactates (in particular derived from L-(+)-lactic acid), tartrates (in particular derived from L-(+)-tartaric acid) and trifluoroacetates. In yet a further particular embodiment, the present invention provides compounds according to formula (I) as described herein (i.e., as “free bases” or “free acids”, respectively). In some embodiments, the pharmaceutically acceptable salt is formate.

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. ²H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

Processes of Manufacturing

The preparation of compounds of formula (I) of the present invention may be carried out in sequential or convergent synthetic routes. Syntheses of the compounds of the invention are shown in the following scheme. The skills required for carrying out the reactions and purifications of the resulting products are known to those skilled in the art. The substituents and indices used in the following description of the processes have the significance given herein before unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). We find it convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

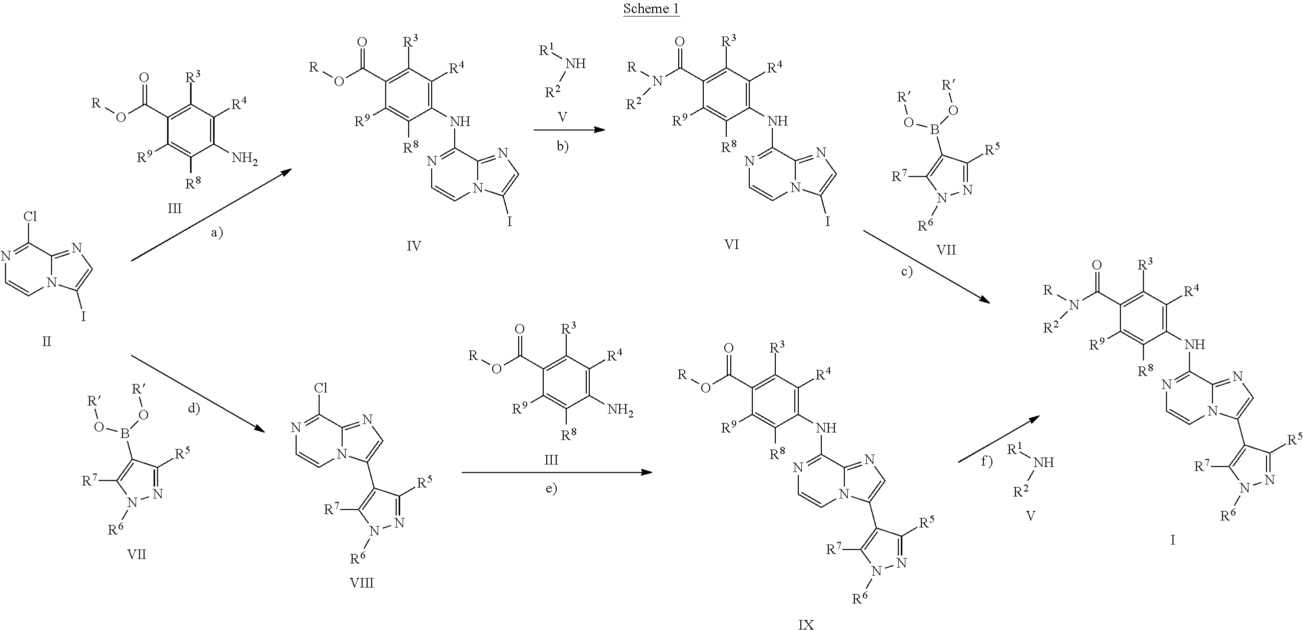

a) 8-chloro-3-iodoimidazo[1,2-a]pyrazine II is commercially available and can conveniently be reacted with aniline derivatives III under acidic or basic conditions in the presence or absence of a transition metal catalyst (depending on the nature and hence the reactivity of the chosen aniline derivative III) in a suitable solvent, depending on the reagent chosen to access imidazo-pyrazine derivative IV. In case R=alkyl, the ester functionality can be cleaved under suitable acidic or basic conditions to access acid derivatives III.

b) Acid or ester derivatives III can conveniently be reacted with amines V (primary, secondary or additionally protected bis-amines) in presence of a coupling reagent (HATU, TBTU, and the like) in the presence of a solvent (DMF, dioxane, THF, and the like), in the presence of a base (DIPEA, triethyl amine, and the like) to access imidazopyridazines VI. These compounds can be intermediates utilized in the subsequent Suzuki reaction, however, in case as amine VI was chosen an additionally protected bis-amines, the protecting group can be cleaved under appropriate conditions and the liberated amine functionality further derivatised to form amides or ureas.

c) Imidazopyrazines VI can conveniently be engaged in a Suzuki reaction with boronic acids or esters VII under transition metal catalysis (typical metal source: Pd and the like) in a solvent (dioxane, DMF, THF and the like) in the presence of abase (NEt₃, DIPEA, carbonates, and the like) to yield imidazopyrazine derivatives I. These might be the final compounds, however further transformation at the pyrazole ring system allow for access to final imidazopyrazine derivatives I or further transformation at the amide moiety allow access to final imidazopyrazine derivatives I.

d) 8-chloro-3-iodoimidazo[1,2-a]pyrazine I is commercially available and can conveniently be engaged in a Suzuki reaction with boronic acids or esters VII under transition metal catalysis (typical metal source: Pd and the like) in a solvent (dioxane, DMF, THF and the like) in the presence of a base (NEt₃, DIPEA, carbonates, and the like) to yield imidazopyrazine derivatives VIII.

e) Imidazopyrazine derivatives VIII can conveniently be reacted with aniline derivatives III under acidic or basic conditions in the presence or absence of a transition metal catalyst (depending on the nature and hence the reactivity of the chosen aniline derivative III) in a suitable solvent, depending on the reagent chosen to access imidazo-pyrazine derivative IV. In case R=alkyl, the ester functionality can be cleaved under suitable acidic or basic conditions to access acid derivatives IX.

f) Acid or ester derivatives IX can conveniently be reacted with amines V (primary, secondary or additionally protected bis-amines) in presence of a coupling reagent (HATU, TBTU, and the like) in the presence of a solvent (DMF, dioxane, THF, and the like), in the presence of a base (DIPEA, triethyl amnine, and the like) to access imidazopyridazines I. These might be the final compounds, however further transformation at the pyrazole ring system allow for access to final imidazopyrazine derivatives I. Also in case as amine VI was chosen an additionally protected bis-amines, the protecting group can be cleaved under appropriate conditions and the liberated amine functionality further derivatised to form amides or ureas as final imidazopyrazine derivatives I.

Compounds with the following substructure

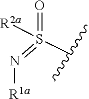

can be synthesized as depicted in Scheme 2:

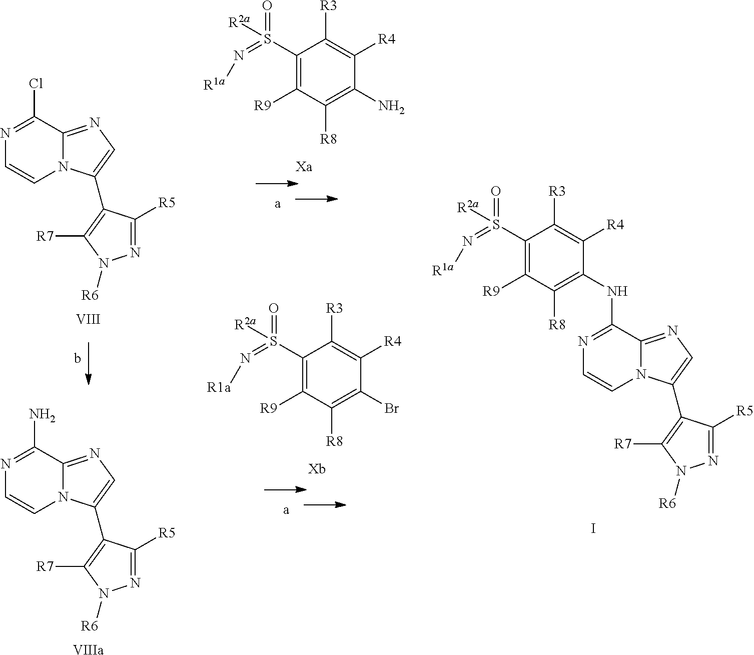

a) Intermediates of type VIII can be reacted with Intermediates of type Xa as described in Scheme 1, conditions a. Those are then followed by other potentially necessary steps as described in Scheme 1, e.g. deprotections. In this context, when R⁶═H, a suitable protecting group in this position might be used. When starting from VIIIa, the transition metal catalized reactions are preferred for the initial step.

b) Intermediates of type VIII can be transformed to Intermediates of type VIIIa by heating them with aq. ammonia in a sealed apparatus in a suitable solvent.

Intermediates Xa and Xb can be synthesized as shown in Scheme 3:

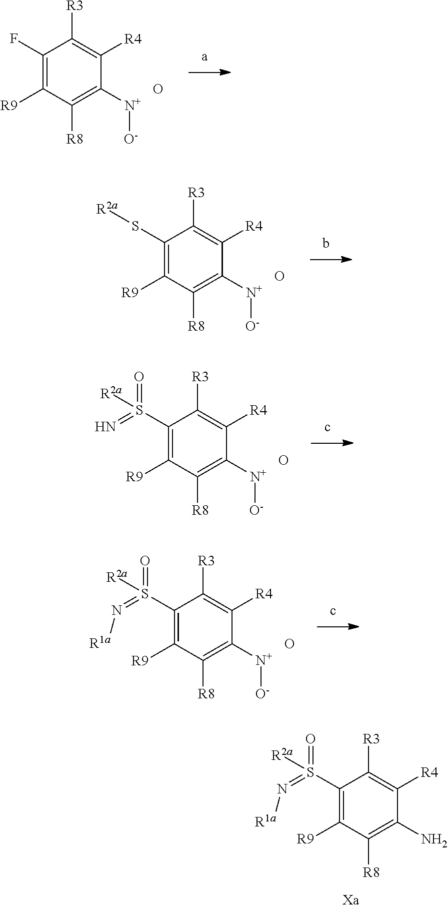

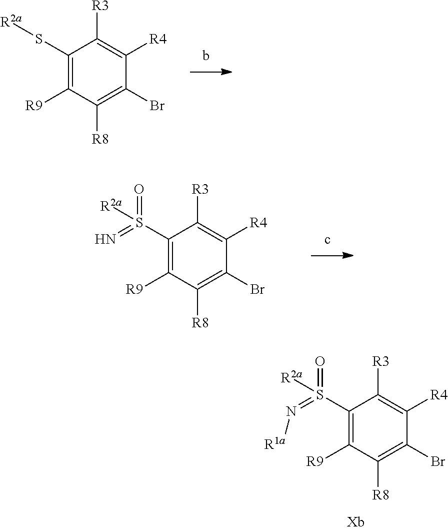

a) 4-fluoronitrobenzenes can be reacted with corresponding sulfides or sulfide salts in an appropriate solvent, usually at elevated temperatures.

b) obtained thioethers can then be transformed to the corresponding sulfoximines by reacting them with ammonium carbamate and iodobenzene diacetate in a suitable solvent, usually at around or slightly below room temperature.

c) obtained sulfoximines can then be alkylated by using the corresponding alkyl halides in the presence of a suitable base in a suitable solvent, usually at elevated temperatures.

d) The still present nitro group can be reduced to the corresponding aniline in a number of ways. One possibility is the reaction with sodium borohydride in the presence of nickel(II) chloride in a suitable solvent.

In one aspect, the present invention provides a process of manufacturing the compounds of formula (I) described herein, comprising:
(i) reacting a carboxylic acid IXa, wherein R³ to R⁹ are as defined herein,

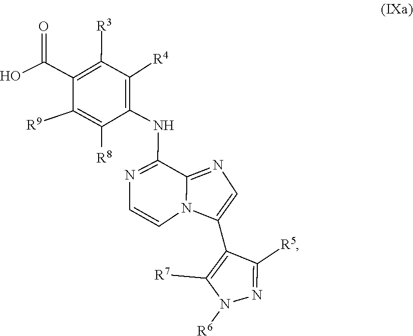

with an amine V, wherein R¹ and R² are as defined herein,

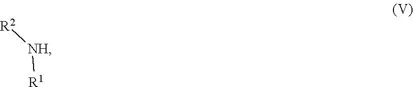

in the presence of a coupling reagent (such as HATU, TBTU, and the like) and a base (such as DIPEA, NEt₃, and the like), optionally in a solvent (such as DMF, dioxane, THF, and the like) to form said compound of formula (I); or
(ii) reacting a compound VIa, wherein R¹ to R⁴, R⁸, and R⁹ are as defined herein and Z is a halogen or a triflate,

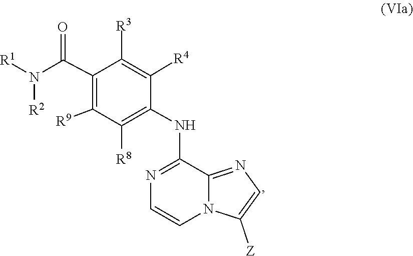

with a compound VIIa, wherein R⁵ to R⁷ are as defined herein and Y is a boronic acid or a boronic acid ester,

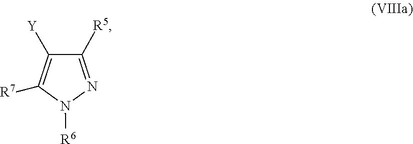

in the presence of a transition metal catalyst (such as PdCl₂(dppf)-CH₂Cl₂ adduct, Pd(PPh₃)₄, and the like) and a base (such as NEt₃, DIPEA, carbonates, K₃PO₄, NaOtBu, and the like) in a solvent (such as dioxane, DMF, THF and the like) to form said compound of formula (I).

In a further aspect, the present invention provides a compound of formula (I) as described herein, or a pharmaceutically acceptable salt thereof, when manufactured according to the processes disclosed herein.

Using the Compounds

As illustrated in the experimental section, the compounds of formula (I) and their pharmaceutically acceptable salts possess valuable pharmacological properties for the treatment or prevention of infections and resulting diseases, particularly bacteremia, pneumonia, meningitis, urinary tract infection, and wound infection, caused by pathogens, particularly by bacteria, more particularly by Acinetobacter species, most particularly by Acinetobacter baumannii.

The compounds of formula (I) and their pharmaceutically acceptable salts exhibit activity as antibiotics, particularly as antibiotics against Acinetobacter species, more particularly as antibiotics against Acinetobacter baumannii, most particularly as pathogen-specific antibiotics against Acinetobacter baumannii.

The compounds of formula (I) and their pharmaceutically acceptable salts can be used as antibiotics, i.e. as antibacterial pharmaceutical ingredients suitable in the treatment and prevention of bacterial infections, particularly in the treatment and prevention of bacterial infections caused by Acinetobacter species, more particularly in the treatment and prevention of bacterial infections caused by Acinetobacter baumannii.

The compounds of the present invention can be used, either alone or in combination with other drugs, for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In one aspect, the present invention provides compounds of formula (I) or their pharmaceutically acceptable salts as described herein for use as therapeutically active substances.

In a further aspect, the present invention provides a compound of formula (I) as described herein, or a pharmaceutically acceptable salt thereof, for use as antibiotic.

In a further aspect, the present invention provides a compound of formula (I) as described herein, or a pharmaceutically acceptable salt thereof, for use in the treatment or prevention of nosocomial infections and resulting diseases.

In a particular embodiment, said nosocomial infections and resulting diseases are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Gram-negative bacteria.

In a particular embodiment, said infections and resulting diseases caused by Gram-negative bacteria are selected from bacteremia, pneumonia, meningitis, urinary tract infection and wound infection, or a combination thereof.

In a further aspect, the present invention provides a compound of formula (I) as described herein, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides a method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) as described herein, or a pharmaceutically acceptable salt thereof, to a mammal.

In a further aspect, the present invention provides the use of a compound of formula (I) as described herein, or a pharmaceutically acceptable salt thereof, as an antibiotic.

In a further aspect, the present invention provides the use of a compound of formula (I) as described herein, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a further aspect, the present invention provides the use of a compound of formula (I) as described herein, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In a particular embodiment, said infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, are selected from bacteremia, pneumonia, meningitis, urinary tract infection and wound infection, or a combination thereof.

In a further aspect, the present invention provides compounds of formula (I) or their pharmaceutically acceptable salts as defined above for use in the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides a method for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii, which method comprises administering a compound of formula (I) or a pharmaceutically acceptable salt thereof as defined above to a mammal.

In a further aspect, the present invention provides the use of compounds of formula (I) or their pharmaceutically acceptable salts as defined above for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

In a further aspect, the present invention provides the use of compounds of formula (I) or their pharmaceutically acceptable salts as defined above for the preparation of medicaments for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii. Such medicaments comprise compounds of formula (I) or their pharmaceutically acceptable salts as defined above.

Pharmaceutical Compositions and Administration

In one aspect, the present invention provides pharmaceutical compositions comprising compounds of formula (I) or their pharmaceutically acceptable salts as defined above and one or more pharmaceutically acceptable excipients. Exemplary pharmaceutical compositions are described in Examples 834 to 837.

In a further aspect, the present invention relates to pharmaceutical compositions comprising compounds of formula (I) or their pharmaceutically acceptable salts as defined above and one or more pharmaceutically acceptable excipients for the treatment or prevention of infections and resulting diseases, particularly bacteremia, pneumonia, meningitis, urinary tract infection, and wound infection, caused by pathogens, particularly by bacteria, more particularly caused by Acinetobacter species, most particularly by Acinetobacter baumannii.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragées, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions or infusion solutions).

The compounds of formula (I) and their pharmaceutically acceptable salts can be processed with pharmaceutically inert, inorganic or organic excipients for the production of tablets, coated tablets, dragees and hard gelatin capsules. Lactose, corn starch or derivatives thereof, talc, stearic acid or its salts etc. can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

Co-Administration of Compounds of Formula (I) and Other Agents

The compounds of formula (I) or salts thereof or a compound disclosed herein or a pharmaceutically acceptable salt thereof may be employed alone or in combination with other agents for treatment. For example, the second agent of the pharmaceutical combination formulation or dosing regimen may have complementary activities to the compound of formula (I) such that they do not adversely affect each other. The compounds may be administered together in a unitary pharmaceutical composition or separately. In one embodiment a compound or a pharmaceutically acceptable salt can be co-administered with an antibiotic, in particular with an antibiotic for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

The term “co-administering” refers to either simultaneous administration, or any manner of separate sequential administration, of a compound of formula (I) or a salt thereof or a compound disclosed herein or a pharmaceutically acceptable salt thereof and a further active pharmaceutical ingredient or ingredients, including antibiotic agents. If the administration is not simultaneous, the compounds are administered in a close time proximity to each other. Furthermore, it does not matter if the compounds are administered in the same dosage form, e.g. one compound may be administered intravenously and another compound may be administered orally.

Typically, any agent that has antimicrobial activity may be co-administered. Particular examples of such agents are Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified e.g. in WO 2017072062 A1, WO 2019185572 A1 and WO 2019206853 A1, and Macrolides (erythromycin).

In one aspect, the present invention provides a pharmaceutical composition described herein, further comprising an additional therapeutic agent.

In one aspect, the present invention provides a pharmaceutical combination comprising a compound of formula (I) described herein and an additional therapeutic agent.

In one embodiment, said additional therapeutic agent is an antibiotic agent.

In one embodiment, said additional therapeutic agent is an antibiotic agent that is useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

In one embodiment, said additional therapeutic agent is an antibiotic agent selected from Carbapenems (meropenem), Fluoroquinolone (Ciprofloxacin), Aminoglycoside (amikacin), Tetracyclines (tigecycline), Colistin, Sulbactam, Sulbactam+Durlobactam, Cefiderocol (Fetroja), macrocyclic peptides as exemplified in WO 2017072062 A1, WO 2019185572 A1 and WO 2019206853 A1, and Macrolides (erythromycin).

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### N-[2-(2-aminoethoxy)ethyl]-2-ethyl-4-[[3-[1-methyl-3-(trifluoromethyl)pyrazol-4-yl]imidazo[1,2-a]pyrazin-8-yl]amino]benzamide

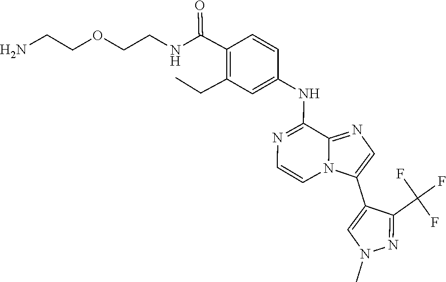 [SMILES: CCc1cc(Nc2nccn3c(-c4cn(C)nc4C(F)(F)F)cnc23)ccc1C(=O)NCCOCCN]

Step 1:

### methyl 2-ethyl-4-[(3-iodoimidazo[1,2-a]pyrazin-8-yl)amino]benzoate

A mixture of 8-chloro-3-iodoimidazo[1,2-a]pyrazine (18 g, 64.4 mmol) and methyl 4-amino-2-ethylbenzoate (13.3 g, 74.1 mmol) in acetonitrile (140 mL) and acetic acid (14.7 g, 14 mL) was heated to 85° C. After cooling to room temperature the precipitate was filtered off, washed with acetonitrile/methanol 1:1 and dried to yield the title compound as off-white crystals (28.6 g, 62.4 mmol, 97%). MS(m/e): 423.1 (M+H).

Step 2:

### 2-ethyl-4-[(3-iodoimidazo[1,2-a]pyrazin-8-yl)amino]benzoic acid, hydrochloride

A mixture of methyl 2-ethyl-4-((3-iodoimidazo[1,2-a]pyrazin-8-yl)amino)benzoate (1.48 g, 3.23 mmol) and LiOH·H₂O (677 mg, 16.1 mmol) in THF (30 mL), water (15 mL) and MeOH (15 mL) was heated to 60° C. and stirred overnight. The reaction mixture was concentrated in vacuo and poured into 30 mL 1 M HCl. The precipitate was filtered through sintered glass, washed with water and dried under high vacuum for 2 h to give the title compound as white solid (1.421 g, 3.2 mmol, 99%). MS(m/e): 409.1 (M+H).

Step 3:

### tert-butyl N-[2-[2-[[2-ethyl-4-[(3-iodoimidazo[1,2-a]pyrazin-8-yl)amino]benzoyl]amino]ethoxy]ethyl]carbamate

A mixture of 2-ethyl-4-((3-iodoimidazo[1,2-a]pyrazin-8-yl)amino)benzoic acid hydrochloride (1.421 g, 3.2 mmol), TBTU (1.22 g, 3.68 mmol), tert-butyl (2-(2-aminoethoxy)ethyl)carbamate (816 mg, 3.99 mmol) and triethylamine (1.62 g, 2.23 mL, 16 mmol,) in DMF (20 mL) was stirred at room temperature overnight. The reaction mixture was poured into 150 mL water and extracted with ethyl acetate (2×100 mL). The crude material was adsorbed on Isolute and purified by flash column chromatography (silica gel, 80 g, 0% to 100% ethyl acetate in heptane). Evaporation of the product containing fractions yielded the title compound (1.561 g, 2.63 mmol, 82.2%). MS(m/e): 595.4 (M+H).

Step 4:

### tert-butyl N-[2-[2-[[2-ethyl-4-[[3-[1-methyl-3-(trifluoromethyl)pyrazol-4-yl]imidazo[1,2-a]pyrazin-8-yl]amino]benzoyl]amino]ethoxy]ethyl]carbamate

A mixture of 1-methyl-4-(4,4,5,5-tetramethyl-1,3,2-dioxaborolan-2-yl)-3-(trifluoromethyl)-1H-pyrazole (29 mg, 105 μmol), tert-butyl N-[2-[2-[[2-ethyl-4-[(3-iodoimidazo[1,2-a]pyrazin-8-yl)amino]benzoyl]amino]ethoxy]ethyl]carbamate (41.6 mg, 70 μmol), Na₂CO₃ (14.8 mg, 140 μmol) and 1,1′-bis(diphenylphosphino)ferrocene-palladium(II)dichloride dichloromethane adduct (5.72 mg, 7 μmol) in dioxane (1 mL)/water (100 μL) was stirred at 110° C. overnight.

The reaction mixture were poured into 4 mL water, extracted with ethyl acetate (2×5 mL) and concentrated. The title compound was used without further purification in the subsequent step. MS(m/e): 617.4 (M+H).

Step 5:

### N-[2-(2-aminoethoxy)ethyl]-2-ethyl-4-[[3-[1-methyl-3-(trifluoromethyl)pyrazol-4-yl]imidazo[1,2-a]pyrazin-8-yl]amino]benzamide

tert-butyl N-[2-[2-[[2-ethyl-4-[[3-[1-methyl-3-(trifluoromethyl)pyrazol-4-yl]imidazo[1,2-a]pyrazin-8-yl]amino]benzoyl]amino]ethoxy]ethyl]carbamate (crude) was dissolved in DCM (2 mL) and treated with an excess HCl in dioxane (525 uL, 4N) and stirred at room temperature overnight. The mixture was evaporated, dissolved in DMF and purified by column chromatography on reversed phase eluting with a gradient formed from acetonitrile/water/trimethylamine. The product containing fractions were evaporated to yield the title compound (5.2 mg, 14% over two steps). MS(m/e): 517.4 (M+H).

### Example 2

### N-[2-(2-aminoethoxy)ethyl]-2-ethyl-4-[[3-[1-(pyrimidin-4-ylmethyl)-3-(trifluoromethyl)pyrazol-4-yl]imidazo[1,2-a]pyrazin-8-yl]amino]benzamide

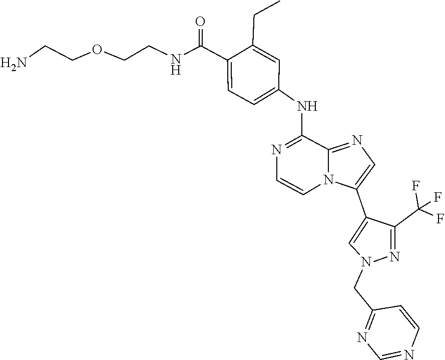 [SMILES: CCc1cc(Nc2nccn3c(-c4cn(Cc5ccncn5)nc4C(F)(F)F)cnc23)ccc1C(=O)NCCOCCN]

Step 1:

### tert-butyl N-[2-[2-[[2-ethyl-4-[[3-[3-(trifluoromethyl)-1H-pyrazol-4-yl]imidazo[1,2-a]pyrazin-8-yl]amino]benzoyl]amino]ethoxy]ethyl]carbamate

In analog to the procedure described for the synthesis of tert-butyl N-[2-[2-[[2-ethyl-4-[[3-[1-methyl-3-(trifluoromethyl)pyrazol-4-yl]imidazo[1,2-a]pyrazin-8-yl]amino]benzoyl]amino]ethoxy]ethyl]carbamate (example 1, step 4) the title compound was prepared from tert-butyl N-[2-[2-[[2-ethyl-4-[(3-iodoimidazo[1,2-a]pyrazin-8-yl)amino]benzoyl]amino]ethoxy]ethyl]carbamate and (3-(trifluoromethyl)-1H-pyrazol-4-yl)boronic acid as light yellow oil. MS(m/e): 603.3 (M+H).

Step 2:

### tert-butyl N-[2-[2-[[2-ethyl-4-[[3-[1-(pyrimidin-4-ylmethyl)-3-(trifluoromethyl)pyrazol-4-yl]imidazo[1,2-a]pyrazin-8-yl]amino]benzoyl]amino]ethoxy]ethyl]carbamate

A mixture of tert-butyl N-[2-[2-[[2-ethyl-4-[[3-[3-(trifluoromethyl)-1H-pyrazol-4-yl]imidazo[1,2-a]pyrazin-8-yl]amino]benzoyl]amino]ethoxy]ethyl]carbamate (40.1 mg, 56.5 μmol), potassium carbonate (19.5 mg, 141 μmol) and 4-(bromomethyl)pyrimidine hydrobromide (21.5 mg, 84.7 μmol) in DMF (2 mL) was stirred at room temperature overnight and heated to 70° C. After cooling the mixture was poured into water (15 mL) and extracted with ethyl acetate (2×15 mL). The combined organic layers were dried over MgSO₄ and evaporated. The crude title compound was used without further purification in the subsequent step. MS(m/e): 695.3 (M+H).

Step 3:

### N-[2-(2-aminoethoxy)ethyl]-2-ethyl-4-[[3-[1-(pyrimidin-4-ylmethyl)-3-(trifluoromethyl)pyrazol-4-yl]imidazo[1,2-a]pyrazin-8-yl]amino]benzamide

A mixture of tert-butyl N-[2-[2-[[2-ethyl-4-[[3-[1-(pyrimidin-4-ylmethyl)-3-(trifluoromethyl)pyrazol-4-yl]imidazo[1,2-a]pyrazin-8-yl]amino]benzoyl]amino]ethoxy]ethyl]carbamate and TFA (0.15 mL) in DCM (2 mL) was stirred at room temperature for 2 h and concentrated in vacuo. The residue was purified by column chromatography on reversed phase eluting with a gradient formed from acetonitrile/water/trimethylamine and the product containing fractions were evaporated to yield the title compound (5.2 mg, 15% over two steps). MS(m/e): 595.2 (M+H).

### Example 3

### (2S)—N-[2-[[4-[[3-[1-(cyanomethyl)-3-(trifluoromethyl)pyrazol-4-yl]imidazo[1,2-a]pyrazin-8-yl]amino]-2-ethylbenzoyl]amino]ethyl]pyrrolidine-2-carboxamide

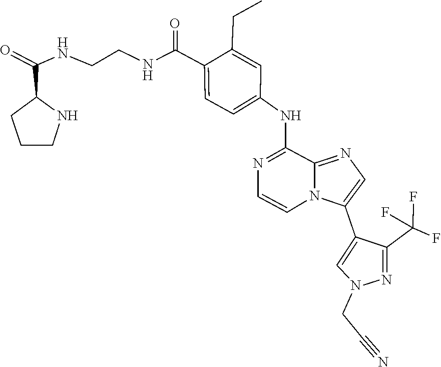 [SMILES: CCc1cc(Nc2nccn3c(-c4cn(CC#N)nc4C(F)(F)F)cnc23)ccc1C(=O)NCCNC(=O)[C@@H]1CCCN1]

Step 1:

### methyl 2-ethyl-4-[[3-[3-(trifluoromethyl)-1H-pyrazol-4-yl]imidazo[1,2-a]pyrazin-8-yl]amino]benzoate

In analogy to the procedure described for the synthesis of tert-butyl N-[2-[2-[[2-ethyl-4-[[3-[1-methyl-3-(trifluoromethyl)pyrazol-4-yl]imidazo[1,2-a]pyrazin-8-yl]amino]benzoyl]amino]ethoxy]ethyl]carbamate (example 1, step 4) the title compound was prepared from methyl 2-ethyl-4-[(3-iodoimidazo[1,2-a]pyrazin-8-yl)amino]benzoate and (3-(trifluoromethyl)-1H-pyrazol-4-yl)boronic acid as light red solid. MS(m/e): 433.3 (M+H).

Step 2:

### 2-ethyl-4-[[3-[3-(trifluoromethyl)-1H-pyrazol-4-yl]imidazo[1,2-a]pyrazin-8-yl]amino]benzoic acid, hydrochloric acid

In analogy to the procedure described for the synthesis of 2-ethyl-4-[(3-iodoimidazo[1,2-a]pyrazin-8-yl)amino]benzoic acid (example 1, step 2) the title compounds was prepared from methyl 2-ethyl-4-[[3-[3-(trifluoromethyl)-1H-pyrazol-4-yl]imidazo[1,2-a]pyrazin-8-yl]amino]benzoate as light grey solid. MS(m/e): 417.2 (M+H).

Step 3:

### tert-butyl N-[2-[[2-ethyl-4-[[3-[3-(trifluoromethyl)-1H-pyrazol-4-yl]imidazo[1,2-a]pyrazin-8-yl]amino]benzoyl]amino]ethyl]carbamate

A mixture of 2-ethyl-4-((3-(3-(trifluoromethyl)-1H-pyrazol-4-yl)imidazo[1,2-a]pyrazin-8-yl)amino)benzoic acid hydrochloride (1.343 g, 2.97 mmol) was dissolved in DMF (15.3 mL). N-ethyl-N-isopropylpropan-2-amine (1.92 g, 2.52 mL, 14.8 mmol), tert-butyl (2-aminoethyl)carbamate CAS [57260-73-8] (570 mg, 3.56 mmol) and 2-(3H-[1,2,3]triazolo[4,5-b]pyridin-3-yl)-1,1,3,3-tetramethylisouronium hexafluorophosphate(V) (HATU) (2.26 g, 5.93 mmol) were added and the yellow solution was stirred at room temperature for 1 h. The solvent was evaporated and the crude material was purified by flash chromatography (silica gel, 100 g, 0% to 100% DCM/MeOH/NH₄OH (90/10/1)). The product containing fractions were evaporated to yield the title compound as brown solid (2.324 g, purity 70%) and used in the subsequent step without further purification. MS(m/e): 559.4 (M+H).

Step 4:

### N-(2-aminoethyl)-2-ethyl-4-[[3-[3-(trifluoromethyl)-1H-pyrazol-4-yl]imidazo[1,2-a]pyrazin-8-yl]amino]benzamide

In analogy to the procedure described for the synthesis of N-[2-(2-aminoethoxy)ethyl]-2-ethyl-4-[[3-[1-(pyrimidin-4-ylmethyl)-3-(trifluoromethyl)pyrazol-4-yl]imidazo[1,2-a]pyrazin-8-yl]amino]benzamide (example 2, step 3) the title compound was prepared from tert-butyl N-[2-[[2-ethyl-4-[[3-[3-(trifluoromethyl)-1H-pyrazol-4-yl]imidazo[1,2-a]pyrazin-8-yl]amino]benzoyl]amino]ethyl]carbamate. MS(m/e): 459.3 (M+H).

Step 5:

### tert-butyl (2S)-2-[2-[[2-ethyl-4-[[3-[3-(trifluoromethyl)-1H-pyrazol-4-yl]imidazo[1,2-a]pyrazin-8-yl]amino]benzoyl]amino]ethylcarbamoyl]pyrrolidine-1-carboxylate

A mixture of N-(2-aminoethyl)-2-ethyl-4-((3-(3-(trifluoromethyl)-1H-pyrazol-4-yl)imidazo[1,2-a]pyrazin-8-yl)amino)benzamide (660 mg, 1.44 mmol) was dissolved in DMF (9 mL). N-ethyl-N-isopropylpropan-2-amine (930 mg, 1.22 ml, 7.2 mmol), (tert-butoxycarbonyl)-L-proline CAS [15761-39-4] (372 mg, 1.73 mmol) and 2-(3H-[1,2,3]triazolo[4,5-b]pyridin-3-yl)-1,1,3,3-tetramethylisouronium hexafluorophosphate(V) (HATU) (1.09 g, 2.88 mmol) were added and the yellow solution was stirred at room temperature for 1.5 h. The solvent was evaporated and the crude material was purified by flash chromatography (silica gel, 100 g, 0% to 100% DCM/MeOH/NH₄OH (90/10/1)). The product containing fractions were evaporated to yield the title compound as white foam (1 g, 96% purity) and used in the subsequent step without further purification. MS(m/e): 656.4 (M+H).

Step 6:

### tert-butyl (2S)-2-[2-[[4-[[3-[1-(cyanomethyl)-3-(trifluoromethyl)pyrazol-4-yl]imidazo[1,2-a]pyrazin-8-yl]amino]-2-ethyl-benzoyl]amino]ethylcarbamoyl]pyrrolidine-1-carboxylate

In analogy to the alkylation procedure described for the synthesis of tert-butyl N-[2-[2-[[2-ethyl-4-[[3-[1-(pyrimidin-4-ylmethyl)-3-(trifluoromethyl)pyrazol-4-yl]imidazo[1,2-a]pyrazin-8-yl]amino]benzoyl]amino]ethoxy]ethyl]carbamate (example 2, step 2) the title compound was prepared from tert-butyl (2S)-2-[2-[[2-ethyl-4-[[3-[3-(trifluoromethyl)-1H-pyrazol-4-yl]imidazo[1,2-a]pyrazin-8-yl]amino]benzoyl]amino]ethylcarbamoyl]pyrrolidine-1-carboxylate and 2-bromoacetonitrile [CAS #590-17-0]. MS(m/e): 695.5 (M+H).

Step 7:

### (2S)—N-[2-[[4-[[3-[1-(cyanomethyl)-3-(trifluoromethyl)pyrazol-4-yl]imidazo[1,2-a]pyrazin-8-yl]amino]-2-ethylbenzoyl]amino]ethyl]pyrrolidine-2-carboxamide

In analogy to the deprotection procedure described for the synthesis of N-[2-(2-aminoethoxy)ethyl]-2-ethyl-4-[[3-[1-(pyrimidin-4-ylmethyl)-3-(trifluoromethyl)pyrazol-4-yl]imidazo[1,2-a]pyrazin-8-yl]amino]benzamide (example 2, step 3) the title compounds was synthesized from tert-butyl (2S)-2-[2-[[4-[[3-[1-(cyanomethyl)-3-(trifluoromethyl)pyrazol-4-yl]imidazo[1,2-a]pyrazin-8-yl]amino]-2-ethyl-benzoyl]amino]ethylcarbamoyl]pyrrolidine-1-carboxylate and TFA. MS(m/e): 594.6 (M+H).

### Example 4

### 4-[[3-[1-(cyanomethyl)-3-(trifluoromethyl)pyrazol-4-yl]imidazo[1,2-a]pyrazin-8-yl]amino]-2-fluoro-6-methyl-N-prop-2-ynylbenzamide

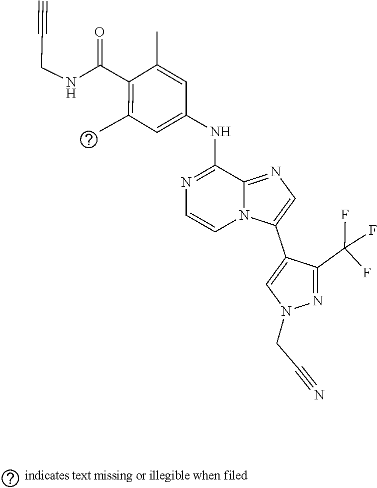 [SMILES: C#CCNC(=O)c1c(C)cc(Nc2nccn3c(-c4cn(CC#N)nc4C(F)(F)F)cnc23)cc1C]

Step 1:

### 8-chloro-3-[3-(trifluoromethyl)-1H-pyrazol-4-yl]imidazo[1,2-a]pyrazine

A mixture of 8-chloro-3-iodoimidazo[1,2-a]pyrazine (3 g, 10.7 mmol), 4-(4,4,5,5-tetramethyl-1,3,2-dioxaborolan-2-yl)-3-(trifluoromethyl)-1H-pyrazole (3.38 g, 12.9 mmol), 1,1′-bis(diphenylphosphino)ferrocene-palladium(II)dichloride dichloromethane adduct (877 mg, 1.07 mmol,) and Na₂CO₃ (2.28 g, 21.5 mmol) in dioxane (107 mL)/water (10.7 mL) was stirred for 5 h at 105° C. The crude material was absorbed with Isolute HM-N, dried and purified by flash chromatography to afford 8-chloro-3-(3-(trifluoromethyl)-1H-pyrazol-4-yl)imidazo[1,2-a]pyrazine as an off-white solid (1.76 g, 6.12 mmol, 57% yield). MS(m/e): 288.2 (M+H).

Step 2:

### methyl 2-fluoro-6-methyl-4-[[3-[3-(trifluoromethyl)-1H-pyrazol-4-yl]imidazo[1,2-a]pyrazin-8-yl]amino]benzoate

A mixture of methyl 4-amino-2-fluoro-6-methylbenzoate (856 mg, 4.67 mmol) and 8-chloro-3-(3-(trifluoromethyl)-1H-pyrazol-4-yl)imidazo[1,2-a]pyrazine (1.583 g, 4.68 mmol) in acetonitrile (16 mL) and acetic acid (1.6 mL) was stirred for 4.5 h at 120° C. The mixture was cooled to ambient temperature. The crude material was absorbed with Isolute HM-N, dried and purified by flash chromatography to afford methyl 2-fluoro-6-methyl-4-((3-(3-(trifluoromethyl)-1H-pyrazol-4-yl)imidazo[1,2-a]pyrazin-8-yl)amino)benzoate (1.833 g, 3.88 mmol, 83.1% yield) as an off-white solid. MS(m/e): 435.2 (M+H).

Step 3:

### 2-fluoro-6-methyl-4-[[3-[3-(trifluoromethyl)-1H-pyrazol-4-yl]imidazo[1,2-a]pyrazin-8-yl]amino]benzoic acid

A mixture of methyl 2-fluoro-6-methyl-4-((3-(3-(trifluoromethyl)-1H-pyrazol-4-yl)imidazo[1,2-a]pyrazin-8-yl)amino)benzoate (1.83 g, 4.21 mmol) in water (20 mL), MeOH (5 mL) and KOH (5M aq) (3 mL, 15 mmol) was stirred for 3.5 h at 70° C. The organic solvent was removed under vavuo. The solution was diluted with water and under stirring a solution of acetic acid (901 mg, 859 μl, 15 mmol) in water was dropwise added at 40° C. The white suspension was cooled to ambient temperature, filtered off and the white crystals washed with water (2×15 mL, 5° C.) and dried under vacuum to afford 2-fluoro-6-methyl-4-((3-(3-(trifluoromethyl)-1H-pyrazol-4-yl)imidazo[1,2-a]pyrazin-8-yl)amino)benzoic acid (1.685 g, 3.65 mmol, 86.6% yield) as white crystals. MS(m/e): 419.3 (M−H).

Step 4:

### 2-fluoro-6-methyl-N-prop-2-ynyl-4-[[3-[3-(trifluoromethyl)-1H-pyrazol-4-yl]imidazo[1,2-a]pyrazin-8-yl]amino]benzamide (example 43)

In analogy to the amide coupling procedure described for the synthesis of example 1, step 3 the title compound was prepared from 2-fluoro-6-methyl-4-[[3-[3-(trifluoromethyl)-1H-pyrazol-4-yl]imidazo[1,2-a]pyrazin-8-yl]amino]benzoic acid and prop-2-yn-1-amine. MS(m/e): 458.2 (M+H).

Step 5:

### 4-[[3-[1-(cyanomethyl)-3-(trifluoromethyl)pyrazol-4-yl]imidazo[1,2-a]pyrazin-8-yl]amino]-2-fluoro-6-methyl-N-prop-2-ynylbenzamide

In analogy to the alkylation procedure described for the synthesis of example 2, step 2 the title compound was prepared from 2-fluoro-6-methyl-N-prop-2-ynyl-4-[[3-[3-(trifluoromethyl)-1H-pyrazol-4-yl]imidazo[1,2-a]pyrazin-8-yl]amino]benzamide and 2-bromoacetonitrile. MS(m/e): 497.2 (M−H).

### Example 5

### [2-ethyl-4-[[3-[1-prop-2-ynyl-3-(trifluoromethyl)pyrazol-4-yl]imidazo[1,2-a]pyrazin-8-yl]amino]phenyl]-[4-(piperidine-4-carbonyl)piperazin-1-yl]methanone

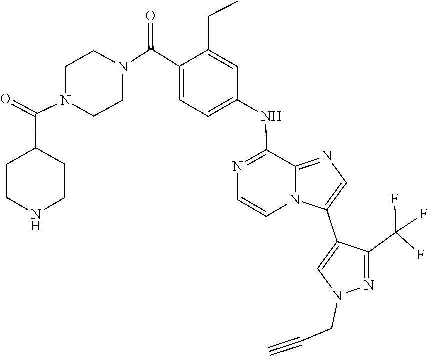 [SMILES: C#CCn1cc(-c2cnc3c(Nc4ccc(C(=O)N5CCN(C(=O)C6CCNCC6)CC5)c(CC)c4)nccn23)c(C(F)(F)F)n1]

Step 1:

### tert-butyl 4-[2-ethyl-4-[(3-iodoimidazo[1,2-a]pyrazin-8-yl)amino]benzoyl]piperazine-1-carboxylate

In analogy to the procedure described for example 1, step 3 the title compound was prepared from 2-ethyl-4-[(3-iodoimidazo[1,2-a]pyrazin-8-yl)amino]benzoic acid, hydrochloride and tert-butyl piperazine-1-carboxylate. MS(m/e): 577.4 (M+H).

Step 2:

### [2-ethyl-4-[(3-iodoimidazo[1,2-a]pyrazin-8-yl)amino]phenyl]-piperazin-1-yl-methanone, hydrochloride

In analogy to the procedure described for example 1, step 5 the title compound was prepared from tert-butyl 4-[2-ethyl-4-[(3-iodoimidazo[1,2-a]pyrazin-8-yl)amino]benzoyl]piperazine-1-carboxylate and tert-butyl piperazine-1-carboxylate through Boc-group cleavage with HCl.

Step 3:

### tert-butyl 4-[4-[2-ethyl-4-[(3-iodoimidazo[1,2-a]pyrazin-8-yl)amino]benzoyl]piperazine-1-carbonyl]piperidine-1-carboxylate

In analogy to the procedure described for example 1, step 3 the title compound was prepared from [2-ethyl-4-[(3-iodoimidazo[1,2-a]pyrazin-8-yl)amino]phenyl]-piperazin-1-yl-methanone, hydrochloride and 1-(tert-butoxycarbonyl)piperidine-4-carboxylic acid. MS(m/e): 588.6 (M+H).

Step 4:

### tert-butyl 4-[4-[2-ethyl-4-[[3-[3-(trifluoromethyl)-1H-pyrazol-4-yl]imidazo[1,2-a]pyrazin-8-yl]amino]benzoyl]piperazine-1-carbonyl]piperidine-1-carboxylate

In analogy to the procedure described for example 1, step 4 the title compound was prepared from tert-butyl 4-[4-[2-ethyl-4-[(3-iodoimidazo[1,2-a]pyrazin-8-yl)amino]benzoyl]piperazine-1-carbonyl]piperidine-1-carboxylate and (3-(trifluoromethyl)-1H-pyrazol-4-yl)boronic acid. MS(m/e): 696.7 (M+H).

Step 5:

### tert-butyl 4-[4-[2-ethyl-4-[[3-[1-prop-2-ynyl-3-(trifluoromethyl)pyrazol-4-yl]imidazo[1,2-a]pyrazin-8-yl]amino]benzoyl]piperazine-1-carbonyl]piperidine-1-carboxylate

In analogy to the procedure described for example 2, step 2 the title compound was prepared from tert-butyl 4-[4-[2-ethyl-4-[[3-[3-(trifluoromethyl)-1H-pyrazol-4-yl]imidazo[1,2-a]pyrazin-8-yl]amino]benzoyl]piperazine-1-carbonyl]piperidine-1-carboxylate and 3-bromoprop-1-yne. MS(m/e): 734.6 (M+H).

Step 6:

### [2-ethyl-4-[[3-[1-prop-2-ynyl-3-(trifluoromethyl)pyrazol-4-yl]imidazo[1,2-a]pyrazin-8-yl]amino]phenyl]-[4-(piperidine-4-carbonyl)piperazin-1-yl]methanone

In analogy to the procedure described for example 2, step 3 the title compound was prepared from tert-butyl 4-[4-[2-ethyl-4-[[3-[1-prop-2-ynyl-3-(trifluoromethyl)pyrazol-4-yl]imidazo[1,2-a]pyrazin-8-yl]amino]benzoyl]piperazine-1-carbonyl]piperidine-1-carboxylate through TFA cleavage of the Boc group. MS(m/e): 634.4 (M+H).

### Example 6

### 2-amino-1-[4-[2-chloro-4-[[3-[1-(2,2-difluoroethyl)-3-(trifluoromethyl)pyrazol-4-yl]imidazo[1,2-a]pyrazin-8-yl]amino]benzoyl]piperazin-1-yl]ethanone

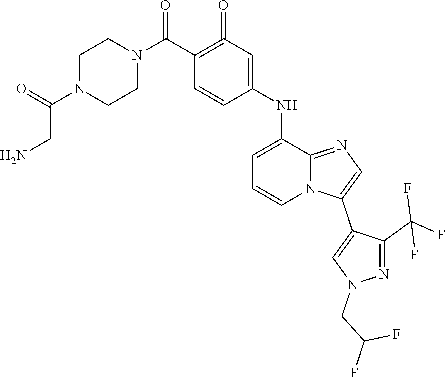

Step 1:

### methyl 2-chloro-4-[(3-iodoimidazo[1,2-a]pyrazin-8-yl)amino]benzoate

In analogy to the procedure described for example 1, step 1 the title compound was prepared from 8-chloro-3-iodoimidazo[1,2-a]pyrazine and methyl 4-amino-2-chloro-benzoate. MS(m/e): 429.2 (M+H).

Step 2:

### methyl 2-chloro-4-[[3-[3-(trifluoromethyl)-1H-pyrazol-4-yl]imidazo[1,2-a]pyrazin-8-yl]amino]benzoate

In analogy to the procedure described for example 1, step 4 the title compound was prepared from methyl 2-chloro-4-[(3-iodoimidazo[1,2-a]pyrazin-8-yl)amino]benzoate and (3-(trifluoromethyl)-1H-pyrazol-4-yl)boronic acid. MS(m/e): 437.2 (M+H).

Step 3:

### 2-chloro-4-[[3-[3-(trifluoromethyl)-1H-pyrazol-4-yl]imidazo[1,2-a]pyrazin-8-yl]amino]benzoic acid

In analogy to the procedure described for example 1, step 2 the title compound was prepared from methyl 2-chloro-4-[[3-[3-(trifluoromethyl)-1H-pyrazol-4-yl]imidazo[1,2-a]pyrazin-8-yl]amino]benzoate. MS(m/e): 423.1 (M+H).

Step 4:

### tert-butyl 4-[2-chloro-4-[[3-[3-(trifluoromethyl)-1H-pyrazol-4-yl]imidazo[1,2-a]pyrazin-8-yl]amino]benzoyl]piperazine-1-carboxylate

In analogy to the procedure described for example 1, step 3 the title compound was prepared from 2-chloro-4-[[3-[3-(trifluoromethyl)-1H-pyrazol-4-yl]imidazo[1,2-a]pyrazin-8-yl]amino]benzoic acid and tert-butyl piperazine-1-carboxylate. MS(m/e): 591.2 (M+H).

Step 5:

### [2-chloro-4-[[3-[3-(trifluoromethyl)-1H-pyrazol-4-yl]imidazo[1,2-a]pyrazin-8-yl]amino]phenyl]-piperazin-1-yl-methanone

In analogy to the procedure described for example 1, step 5 the title compound was prepared from tert-butyl 4-[2-chloro-4-[[3-[3-(trifluoromethyl)-1H-pyrazol-4-yl]imidazo[1,2-a]pyrazin-8-yl]amino]benzoyl]piperazine-1-carboxylate. MS(m/e): 491.2 (M+H).

Step 6:

### tert-butyl N-[2-[4-[2-chloro-4-[[3-[3-(trifluoromethyl)-1H-pyrazol-4-yl]imidazo[1,2-a]pyrazin-8-yl]amino]benzoyl]piperazin-1-yl]-2-oxo-ethyl]carbamate

In analogy to the procedure described for example 5, step 3 the title compound was prepared from [2-chloro-4-[[3-[3-(trifluoromethyl)-1H-pyrazol-4-yl]imidazo[1,2-a]pyrazin-8-yl]amino]phenyl]-piperazin-1-yl-methanone and (tert-butoxycarbonyl)glycine CAS[4530-20-5]. MS(m/e): 648.4 (M+H).

Step 7:

### tert-butyl N-[2-[4-[2-chloro-4-[[3-[1-(2,2-difluoroethyl)-3-(trifluoromethyl)pyrazol-4-yl]imidazo[1,2-a]pyrazin-8-yl]amino]benzoyl]piperazin-1-yl]-2-oxo-ethyl]carbamate

In analogy to the procedure described for example 2, step 2 the title compound was prepared from tert-butyl N-[2-[4-[2-chloro-4-[[3-[3-(trifluoromethyl)-1H-pyrazol-4-yl]imidazo[1,2-a]pyrazin-8-yl]amino]benzoyl]piperazin-1-yl]-2-oxo-ethyl]carbamate and 1,1-difluoro-2-iodo-ethane. MS(m/e): 712.5 (M+H).

Step 8:

In analogy to the procedure described for example 2, step 3 the title compound was prepared from tert-butyl N-[2-[4-[2-chloro-4-[[3-[1-(2,2-difluoroethyl)-3-(trifluoromethyl)pyrazol-4-yl]imidazo[1,2-a]pyrazin-8-yl]amino]benzoyl]piperazin-1-yl]-2-oxo-ethyl]carbamate. MS(m/e): 612.3 (M+H).

### Example 7

### N-[3-[4-(3-aminopropylamino)butylamino]propyl]-2-ethyl-4-[[3-[1-(pyridin-3-ylmethyl)-3-(trifluoromethyl)pyrazol-4-yl]imidazo[1,2-a]pyrazin-8-yl]amino]benzamide hydrochloride

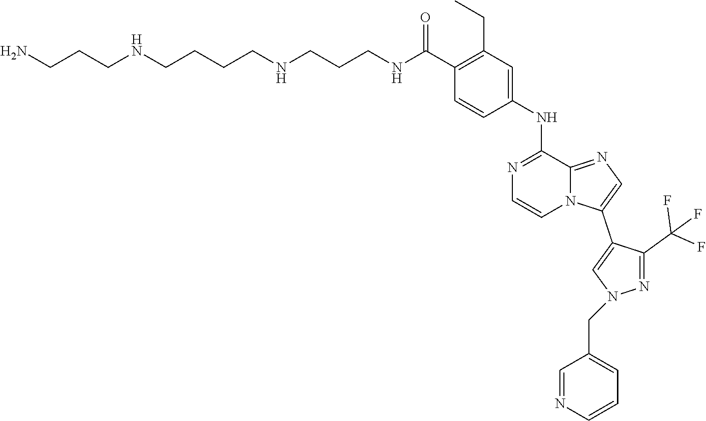 [SMILES: CCc1cc(Nc2nccn3c(-c4cn(Cc5cccnc5)nc4C(F)(F)F)cnc23)ccc1C(=O)NCCCNCCCCNCCCN]

Step 1:

### tert-butyl N-[3-(tert-butoxycarbonylamino)propyl]-N-[4-[tert-butoxycarbonyl-[3-[[2-ethyl-4-[(3-iodoimidazo[1,2-a]pyrazin-8-yl)amino]benzoyl]amino]propyl]amino]butyl]carbamate

In analogy to the procedure described for example 1, step 3 the title compound was prepared from 2-ethyl-4-((3-iodoimidazo[1,2-a]pyrazin-8-yl)amino)benzoic acid hydrochloride and tert-butyl (3-aminopropyl)(4-((tert-butoxycarbonyl)(3-((tert-butoxycarbonyl)amino)propyl)amino)butyl)carbamate. MS(m/e): 893.8 (M+H).

Step 2:

### tert-butyl N-[3-(tert-butoxycarbonylamino)propyl]-N-[4-[tert-butoxycarbonyl-[3-[[2-ethyl-4-[[3-[3-(trifluoromethyl)-1H-pyrazol-4-yl]imidazo[1,2-a]pyrazin-8-yl]amino]benzoyl]amino]propyl]amino]butyl]carbamate

In analogy to the procedure described for example 1, step 4 the title compound was prepared from tert-butyl N-[3-(tert-butoxycarbonylamino)propyl]-N-[4-[tert-butoxycarbonyl-[3-[[2-ethyl-4-[(3-iodoimidazo[1,2-a]pyrazin-8-yl)amino]benzoyl]amino]propyl]amino]butyl]carbamate and (3-(trifluoromethyl)-1H-pyrazol-4-yl)boronic acid. MS(m/e): 899.8 (M−H).

Step 3:

### tert-butyl N-[3-(tert-butoxycarbonylamino)propyl]-N-[4-[tert-butoxycarbonyl-[3-[[2-ethyl-4-[[3-[1-(3-pyridylmethyl)-3-(trifluoromethyl)pyrazol-4-yl]imidazo[1,2-a]pyrazin-8-yl]amino]benzoyl]amino]propyl]amino]butyl]carbamate

In analogy to the procedure described for example 2, step 2 the title compound was prepared from tert-butyl N-[3-(tert-butoxycarbonylamino)propyl]-N-[4-[tert-butoxycarbonyl-[3-[[2-ethyl-4-[[3-[3-(trifluoromethyl)-1H-pyrazol-4-yl]imidazo[1,2-a]pyrazin-8-yl]amino]benzoyl]amino]propyl]amino]butyl]carbamate and 3-(chloromethyl)pyridine hydrochloride. MS(m/e): 992.8 (M+H).

Step 4:

### N-[3-[4-(3-aminopropylamino)butylamino]propyl]-2-ethyl-4-[[3-[1-(pyridin-3-ylmethyl)-3-(trifluoromethyl)pyrazol-4-yl]imidazo[1,2-a]pyrazin-8-yl]amino]benzamide hydrochloride

In analogy to the procedure described for example 1, step 5 the title compound was prepared from tert-butyl N-[3-(tert-butoxycarbonylamino)propyl]-N-[4-[tert-butoxycarbonyl-[3-[[2-ethyl-4-[[3-[1-(3-pyridylmethyl)-3-(trifluoromethyl)pyrazol-4-yl]imidazo[1,2-a]pyrazin-8-yl]amino]benzoyl]amino]propyl]amino]butyl]carbamate. MS(m/e): 692.4 (M+H).

### Example 8

### N-[2-(2-aminoethylamino)-2-oxoethyl]-2-chloro-4-[[3-[1-(2-fluoroethyl)-3-(trifluoromethyl)pyrazol-4-yl]imidazo[1,2-a]pyrazin-8-yl]amino]benzamide formate

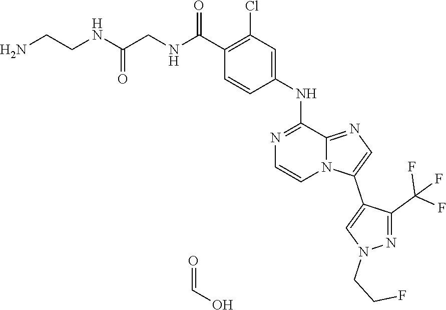 [SMILES: NCCNC(=O)CNC(=O)c1ccc(Nc2nccn3c(-c4cn(CCF)nc4C(F)(F)F)cnc23)cc1Cl ; O=CO]

Step 1:

### ethyl 2-[[2-chloro-4-[[3-[3-(trifluoromethyl)-1H-pyrazol-4-yl]imidazo[1,2-a]pyrazin-8-yl]amino]benzoyl]amino]acetate

In analogy to the procedure described for example 1, step 3 the title compound was prepared from 2-chloro-4-[[3-[3-(trifluoromethyl)-1H-pyrazol-4-yl]imidazo[1,2-a]pyrazin-8-yl]amino]benzoic acid and glycine ethyl ester hydrochloride. MS(m/e): 508.3 (M+H).

Step 2:

### 2-[[2-chloro-4-[[3-[3-(trifluoromethyl)-1H-pyrazol-4-yl]imidazo[1,2-a]pyrazin-8-yl]amino]benzoyl]amino]acetic acid

To a solution of ethyl 2-[[2-chloro-4-[[3-[3-(trifluoromethyl)-1H-pyrazol-4-yl]imidazo[1,2-a]pyrazin-8-yl]amino]benzoyl]amino]acetate (3.5 g, 6.89 mmol) in THF (20 mL)/methanol (20 mL) was added sodium hydroxide aqueous (40.0 mL, 40 mmol) and then stirred at 20° C. for 3 h. The mixture was concentrated, the aqueous layer was washed with ethyl acetate (30 mL) and then adjusted to pH=1-2 by 3N HCl. The precipitate was filtered and dried to yield the title compound as off-white solid (3.2 g, 6.67 mmol, 96.8%). MS(m/e): 480.1 (M+H).

Step 3:

### tert-butyl N-[2-[[2-[[2-chloro-4-[[3-[3-(trifluoromethyl)-1H-pyrazol-4-yl]imidazo[1,2-a]pyrazin-8-yl]amino]benzoyl]amino]acetyl]amino]ethyl]carbamate

In analogy to the procedure described for example 5, step 3 the title compound was prepared from 2-[[2-chloro-4-[[3-[3-(trifluoromethyl)-1H-pyrazol-4-yl]imidazo[1,2-a]pyrazin-8-yl]amino]benzoyl]amino]acetic acid and N—BOC-ethylenediamine. MS(m/e): 622.1 (M+H).

Step 4:

### tert-butyl N-[2-[[2-[[2-chloro-4-[[3-[1-(2-fluoroethyl)-3-(trifluoromethyl)pyrazol-4-yl]imidazo[1,2-a]pyrazin-8-yl]amino]benzoyl]amino]acetyl]amino]ethyl]carbamate

In analogy to the procedure described for example 2, step 2 the title compound was prepared from tert-butyl N-[2-[[2-[[2-chloro-4-[[3-[3-(trifluoromethyl)-1H-pyrazol-4-yl]imidazo[1,2-a]pyrazin-8-yl]amino]benzoyl]amino]acetyl]amino]ethyl]carbamate and 1-bromo-2-fluoroethane. MS(m/e): 668.3 (M+H).

Step 5:

### N-[2-(2-aminoethylamino)-2-oxoethyl]-2-chloro-4-[[3-[1-(2-fluoroethyl)-3-(trifluoromethyl)pyrazol-4-yl]imidazo[1,2-a]pyrazin-8-yl]amino]benzamide formate

In analogy to the procedure described for example 2, step 3 the title compound was prepared from tert-butyl N-[2-[[2-[[2-chloro-4-[[3-[1-(2-fluoroethyl)-3-(trifluoromethyl)pyrazol-4-yl]imidazo[1,2-a]pyrazin-8-yl]amino]benzoyl]amino]acetyl]amino]ethyl]carbamate and TFA. MS(m/e): 568.1 (M+H).

### Example 9

### [2-chloro-4-[difluoromethyl-[3-[3-(trifluoromethyl)-1H-pyrazol-4-yl]imidazo[1,2-a]pyrazin-8-yl]amino]phenyl]-piperazin-1-ylmethanone

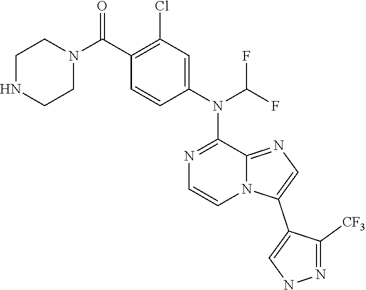 [SMILES: O=C(c1ccc(N(c2nccn3c(-c4c[nH]nc4C(F)(F)F)cnc23)C(F)F)cc1Cl)N1CCNCC1]

Step 1:

### tert-butyl 4-[2-chloro-4-[difluoromethyl-[3-[3-(trifluoromethyl)-1H-pyrazol-4-yl]imidazo[1,2-a]pyrazin-8-yl]amino]benzoyl]piperazine-1-carboxylate

A mixture of tert-butyl 4-(2-chloro-4-((3-(3-(trifluoromethyl)-1H-pyrazol-4-yl)imidazo[1,2-a]pyrazin-8-yl)amino)benzoyl)piperazine-1-carboxylate (example 6, step 4) (200 mg, 338 μmol), sodium hydrogen carbonate [CAS #144-55-8] (85.3 mg, 1.02 mmol) and methyl 2-chloro-2,2-difluoroacetate [CAS #1514-87-0] (97.8 mg, 677 μmol) in DMF (3 mL) was heated in the microwave at 120° C. for 10 min. The crude reaction mixture was purified by reversed phase flash chromatography eluting with a gradient formed from acetonitrile and water. The product containing fractions were evaporated to yield the title compound as yellow solid (52 mg, 16%). MS(m/e): 641.3 (M+H).

Step 2:

### [2-chloro-4-[difluoromethyl-[3-[3-(trifluoromethyl)-1H-pyrazol-4-yl]imidazo[1,2-a]pyrazin-8-yl]amino]phenyl]-piperazin-1-ylmethanone

In analogy to the procedure described for example 2, step 3 the title compound was prepared from tert-butyl 4-[2-chloro-4-[difluoromethyl-[3-[3-(trifluoromethyl)-1H-pyrazol-4-yl]imidazo[1,2-a]pyrazin-8-yl]amino]benzoyl]piperazine-1-carboxylate through TFA cleavage of the Boc group. MS(m/e): 541.3 (M+H).

### Example 10

### 2-chloro-4-[[3-[1-(difluoromethyl)-3-(trifluoromethyl)pyrazol-4-yl]imidazo[1,2-a]pyrazin-8-yl]amino]-N-methylbenzamide

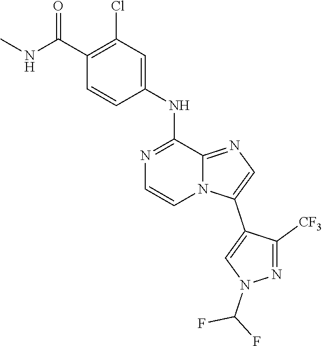 [SMILES: CNC(=O)c1ccc(Nc2nccn3c(-c4cn(C(F)F)nc4C(F)(F)F)cnc23)cc1Cl]

Step 1:

### 2-chloro-4-[(3-iodoimidazo[1,2-a]pyrazin-8-yl)amino]-N-methyl-benzamide

A mixture of 8-chloro-3-iodoimidazo[1,2-a]pyrazine (2 g, 7.16 mmol) and 4-amino-2-chloro-N-methylbenzamide [CAS #926203-17-0] (1.32 g, 7.16 mmol) in acetonitrile (13 mL) and acetic acid glacial (1.3 mL) was heated in the microwave at 120° C. for 30 min. The reaction mixture was diluted with acetonitrile, filtered and the precipitate was washed with acetonitrile (25 mL). The solid was suspended in acetonitrile/water, frozen to −78° C. and lyophilized to yield the title compound as yellow solid (2.83 g, 87%). MS(m/e): 428.1 (M+H).

Step 2:

### 2-chloro-N-methyl-4-[[3-[3-(trifluoromethyl)-1H-pyrazol-4-yl]imidazo[1,2-a]pyrazin-8-yl]amino]benzamide

In analogy to the procedure described for example 1, step 4 the title compound was prepared from 2-chloro-4-[(3-iodoimidazo[1,2-a]pyrazin-8-yl)amino]-N-methyl-benzamide and (3-(trifluoromethyl)-1H-pyrazol-4-yl)boronic acid. MS(m/e): 428.1 (M+H).

Step 3:

### 2-chloro-4-[[3-[1-(difluoromethyl)-3-(trifluoromethyl)pyrazol-4-yl]imidazo[1,2-a]pyrazin-8-yl]amino]-N-methylbenzamide

In analogy to the procedure described for example 2, step 2 the title compound was prepared from 2-chloro-N-methyl-4-[[3-[3-(trifluoromethyl)-1H-pyrazol-4-yl]imidazo[1,2-a]pyrazin-8-yl]amino]benzamide and methyl 2-chloro-2,2-difluoroacetate. MS(m/e): 468.3 (M+H).

### Example 11

### 1-[1-[2-chloro-4-[[3-[1-(cyanomethyl)-3-(trifluoromethyl)pyrazol-4-yl]imidazo[1,2-a]pyrazin-8-yl]amino]benzoyl]piperidin-4-yl]-3-[(3S)-pyrrolidin-3-yl]urea

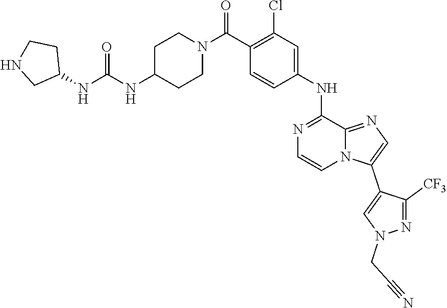

Step 1:

### tert-butyl N-[1-[2-chloro-4-[[3-[3-(trifluoromethyl)-1H-pyrazol-4-yl]imidazo[1,2-a]pyrazin-8-yl]amino]benzoyl]-4-piperidyl]carbamate

In analogy to the procedure described for example 1, step 3 the title compound was prepared from 2-chloro-4-[[3-[3-(trifluoromethyl)-1H-pyrazol-4-yl]imidazo[1,2-a]pyrazin-8-yl]amino]benzoic acid and tert-butyl piperazine-1-carboxylate. MS(m/e): 605.4 (M+H).

Step 2:

### (4-amino-1-piperidyl)-[2-chloro-4-[[3-[3-(trifluoromethyl)-1H-pyrazol-4-yl]imidazo[1,2-a]pyrazin-8-yl]amino]phenyl]methanone

In analogy to the procedure described for example 1, step 5 the title compound was prepared from tert-butyl N-[1-[2-chloro-4-[[3-[3-(trifluoromethyl)-1H-pyrazol-4-yl]imidazo[1,2-a]pyrazin-8-yl]amino]benzoyl]-4-piperidyl]carbamate through Boc deprotection with HCl. MS(m/e): 505.4 (M+H).

Step 3:

### tert-butyl 3-[[1-[2-chloro-4-[[3-[3-(trifluoromethyl)-1H-pyrazol-4-yl]imidazo[1,2-a]pyrazin-8-yl]amino]benzoyl]-4-piperidyl]carbamoylamino]pyrrolidine-1-carboxylate

A mixture of (4-aminopiperidin-1-yl)(2-chloro-4-((3-(3-(trifluoromethyl)-1H-pyrazol-4-yl)imidazo[1,2-a]pyrazin-8-yl)amino)phenyl)methanone (0.15 g, 297 μmol), N-ethyl-N-isopropylpropan-2-amine (192 mg, 253 μl, 1.49 mmol) and triphosgene CAS [32315-10-9] (35.3 mg, 119 μmol) was suspended in DCM (5.99 mL) and stirred at 0° C. for 1 h. A solution of tert-butyl (S)-3-aminopyrrolidine-1-carboxylate [CAS #147081-44-5] (166 mg, 891 μmol) in DCM (2.39 mL) was added and the reaction mixture was stirred at room temperature overnight. Water was added and the mixture was extracted with DCM. The combined organic layer was dried with MgSO₄, filtered and concentrated in vacuo. The crude material was purified by flash chromatography (silica gel, 20 g, 0% to 100% DCM/MeOH/NH₄OH (90/10/1)). The compound was purified a second time to yield after evaporation of the product containing fractions the title compound as colorless gum (0.1 g, 48%). MS(m/e): 717.5 (M+H).

Step 4:

### tert-butyl 3-[[1-[2-chloro-4-[[3-[1-(cyanomethyl)-3-(trifluoromethyl)pyrazol-4-yl]imidazo[1,2-a]pyrazin-8-yl]amino]benzoyl]-4-piperidyl]carbamoylamino]pyrrolidine-1-carboxylate

In analogy to the procedure described for example 2, step 2 the title compound was prepared from tert-butyl 3-[[1-[2-chloro-4-[[3-[3-(trifluoromethyl)-1H-pyrazol-4-yl]imidazo[1,2-a]pyrazin-8-yl]amino]benzoyl]-4-piperidyl]carbamoylamino]pyrrolidine-1-carboxylate and 2-bromoacetonitrile [CAS #590-17-0]. MS(m/e): 756.7 (M+H).

Step 5:

### 1-[1-[2-chloro-4-[[3-[1-(cyanomethyl)-3-(trifluoromethyl)pyrazol-4-yl]imidazo[1,2-a]pyrazin-8-yl]amino]benzoyl]piperidin-4-yl]-3-[(3S)-pyrrolidin-3-yl]urea

In analogy to the procedure described for example 2, step 3 the title compound was prepared from tert-butyl 3-[[1-[2-chloro-4-[[3-[1-(cyanomethyl)-3-(trifluoromethyl)pyrazol-4-yl]imidazo[1,2-a]pyrazin-8-yl]amino]benzoyl]-4-piperidyl]carbamoylamino]pyrrolidine-1-carboxylate through Boc deprotection with TFA. MS(m/e): 656.5 (M+H).

### Example 12

### 2-[4-[8-[4-[4-(3-aminocyclobutanecarbonyl)piperazine-1-carbonyl]-3-chloroanilino]imidazo[1,2-a]pyrazin-3-yl]-3-(trifluoromethyl)pyrazol-1-yl]acetonitrile formate

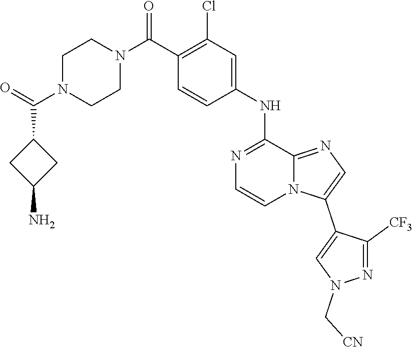 [SMILES: N#CCn1cc(-c2cnc3c(Nc4ccc(C(=O)N5CCN(C(=O)[C@H]6C[C@H](N)C6)CC5)c(Cl)c4)nccn23)c(C(F)(F)F)n1]

Step 1:

### tert-butyl 4-[2-chloro-4-[[3-[1-(cyanomethyl)-3-(trifluoromethyl)pyrazol-4-yl]imidazo[1,2-a]pyrazin-8-yl]amino]benzoyl]piperazine-1-carboxylate

In analogy to the procedure described for example 1, step 4 the title compound was prepared from tert-butyl 4-[2-ethyl-4-[(3-iodoimidazo[1,2-a]pyrazin-8-yl)amino]benzoyl]piperazine-1-carboxylate and 2-[4-(4,4,5,5-tetramethyl-1,3,2-dioxaborolan-2-yl)-3-(trifluoromethyl)pyrazol-1-yl]acetonitrile. MS(m/e): 630.2 (M+H).

Step 2:

### 2-[4-[8-[3-chloro-4-(piperazine-1-carbonyl)anilino]imidazo[1,2-a]pyrazin-3-yl]-3-(trifluoromethyl)pyrazol-1-yl]acetonitrile

In analogy to the procedure described for example 2, step 3 the title compound was prepared from tert-butyl 4-[2-chloro-4-[[3-[1-(cyanomethyl)-3-(trifluoromethyl)pyrazol-4-yl]imidazo[1,2-a]pyrazin-8-yl]amino]benzoyl]piperazine-1-carboxylate through Boc deprotection with TFA. MS(m/e): 530.1 (M+H).

Step 3:

### tert-butyl N-[3-[4-[2-chloro-4-[[3-[1-(cyanomethyl)-3-(trifluoromethyl)pyrazol-4-yl]imidazo[1,2-a]pyrazin-8-yl]amino]benzoyl]piperazine-1-carbonyl]cyclobutyl]carbamate

In analogy to the procedure described for example 5, step 3 the title compound was prepared from 2-[4-[8-[3-chloro-4-(piperazine-1-carbonyl)anilino]imidazo[1,2-a]pyrazin-3-yl]-3-(trifluoromethyl)pyrazol-1-yl]acetonitrile and 3-(tert-butoxycarbonylamino)cyclobutanecarboxylic acid. MS(m/e): 727.3 (M+H).

Step 4:

### 2-[4-[8-[4-[4-(3-aminocyclobutanecarbonyl)piperazine-1-carbonyl]-3-chloroanilino]imidazo[1,2-a]pyrazin-3-yl]-3-(trifluoromethyl)pyrazol-1-yl]acetonitrile formate

In analogy to the procedure described for example 2, step 3 the title compound was prepared from tert-butyl N-[3-[4-[2-chloro-4-[[3-[1-(cyanomethyl)-3-(trifluoromethyl)pyrazol-4-yl]imidazo[1,2-a]pyrazin-8-yl]amino]benzoyl]piperazine-1-carbonyl]cyclobutyl]carbamate through Boc deprotection with TFA. MS(m/e): 627.0 (M+H).

### Example 13

### N-[3-[[(2S)-2-amino-3-carbamimidamidopropanoyl]amino]propyl]-2-ethyl-4-[[3-[3-(trifluoromethyl-1H-pyrazol-4-yl]imidazol[1,2-pyrazin-α]8-yl]amino]benzamide formate

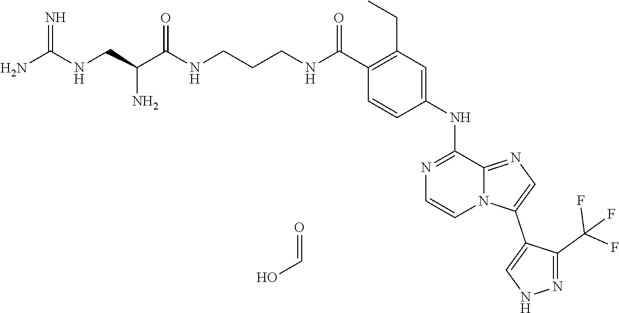 [SMILES: CCc1cc(Nc2nccn3c(-c4c[nH]nc4C(F)(F)F)cnc23)ccc1C(=O)NCCCNC(=O)[C@@H](N)CNC(=N)N ; O=CO]

Step 1:

### tert-butyl N-[3-[[2-ethyl-4-[(3-iodoimidazo[1,2-a]pyrazin-8-yl)amino]benzoyl]amino]propyl]-carbamate

In analogy to the procedure described for example 1, step 3 the title compound was prepared from 2-ethyl-4-[(3-iodoimidazo[1,2-a]pyrazin-8-yl)amino]benzoic acid, hydrochloride and N—BOC-1,3-diaminopropane. MS(m/e): 565.2 (M+H).

Step 2:

### tert-butyl N-[3-[[2-ethyl-4-[[3-[1-[(4-methoxyphenyl)methyl]-3-(trifluoromethyl)pyrazol-4-yl]imidazo[1,2-a]pyrazin-8-yl]amino]benzoyl]amino]propyl]carbamate

In analogy to the procedure described for example 1, step 4 the title compound was prepared from tert-butyl N-[3-[[2-ethyl-4-[(3-iodoimidazo[1,2-a]pyrazin-8-yl)amino]benzoyl]amino]propyl]carbamate and 1-[(4-methoxyphenyl)methyl]-4-(4,4,5,5-tetramethyl-1,3,2-dioxaborolan-2-yl)-3-(trifluoromethyl)pyrazole. MS(m/e): 693.4 (M+H).

Step 3:

### N-(3-aminopropyl)-2-ethyl-4-[[3-[3-(trifluoromethyl)-1H-pyrazol-4-yl]imidazo[1,2-a]pyrazin-8-yl]amino]benzamide

To a solution of tert-butyl N-[3-[[2-ethyl-4-[[3-[1-[(4-methoxyphenyl)methyl]-3-(trifluoromethyl)pyrazol-4-yl]imidazo[1,2-a]pyrazin-8-yl]amino]benzoyl]amino]propyl]-carbamate (680.0 mg, 0.980 mmol) in trifluoroacetic acid (5.0 mL, 64.9 mmol) was added trifluoromethanesulfonic acid (1.47 g, 9.82 mmol,). The reaction was stirred at 80° C. for 2 h. The reaction mixture was concentrated and adjusted to pH=7 with triethyl amine. The residue was purified by preparative-HPLC. The product containing fractions were lyophilized to yield the title compound as white solid (400 mg, 0.850 mmol, 86.3% yield). MS(m/e): 473.2 (M+H).

Step 4:

### tert-butyl N-[(1S)-2-[3-[[2-ethyl-4-[[3-[3-(trifluoromethyl)-1H-pyrazol-4-yl]imidazo[1,2-a]pyrazin-8-yl]amino]benzoyl]amino]propylamino]-1-(guanidinomethyl)-2-oxo-ethyl]carbamate

In analogy to the procedure described for example 5, step 3 the title compound was prepared from N-(3-aminopropyl)-2-ethyl-4-[[3-[3-(trifluoromethyl)-1H-pyrazol-4-yl]imidazo[1,2-a]pyrazin-8-yl]amino]benzamide and (2S)-2-(tert-butoxycarbonylamino)-3-guanidino-propanoic acid. MS(m/e): 701.2 (M+H).

Step 5:

### N-[3-[[(2S)-2-amino-3-carbamimidamidopropanoyl]amino]propyl]-2-ethyl-4-[[3-[3-(trifluoromethyl)-1H-pyrazol-4-yl]imidazo[1,2-a]pyrazin-8-yl]amino]benzamide formate

In analogy to the procedure described for example 2, step 3 the title compound was prepared from tert-butyl N-[(1S)-2-[3-[[2-ethyl-4-[[3-[3-(trifluoromethyl)-1H-pyrazol-4-yl]imidazo[1,2-a]pyrazin-8-yl]amino]benzoyl]amino]propylamino]-1-(guanidinomethyl)-2-oxo-ethyl]carbamate through Boc deprotection with TFA. MS(m/e): 601.3 (M+H).

### Example 14

### 2-[4-[8-[3-chloro-4-[4-hydroxy-4-(methylaminomethyl)piperidine-1-carbonyl]anilino]imidazo[1,2-a]pyrazin-3-yl]-3-(trifluoromethyl)pyrazol-1-yl]acetonitrile formate

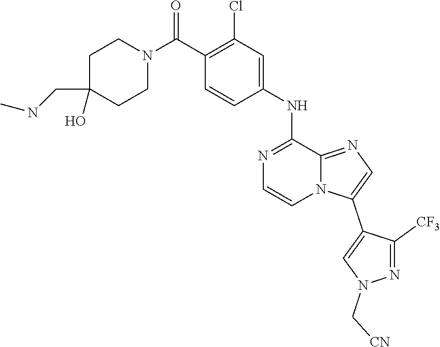 [SMILES: CNCC1(O)CCN(C(=O)c2ccc(Nc3nccn4c(-c5cn(CC#N)nc5C(F)(F)F)cnc34)cc2Cl)CC1]

Step 1:

### 2-chloro-4-[(3-iodoimidazo[1,2-a]pyrazin-8-yl)amino]benzoic acid

To a solution of methyl 2-chloro-4-[(3-iodoimidazo[1,2-a]pyrazin-8-yl)amino]benzoate (25.0 g, 58.33 mmol) (example 6, step 1) in THF (125 mL)/methanol (125 mL) was added sodium hydroxide aqueous (125.0 mL, 250 mmol) and stirred at 60° C. for 12 h. The cooled reaction mixture was adjusted to pH=1-2 by 3N HCl, filtered and evaporated to yield the crude title compound as white solid (25 g) as white solid. MS(m/e): 414.8 (M+H).

Step 2:

### 2-chloro-4-[[3-[1-(cyanomethyl)-3-(trifluoromethyl)pyrazol-4-yl]imidazo[1,2-a]pyrazin-8-yl]amino]benzoic acid

In analogy to the procedure described for example 1, step 4 the title compound was prepared from 2-chloro-4-[(3-iodoimidazo[1,2-a]pyrazin-8-yl)amino]benzoic acid and 2-[4-(4,4,5,5-tetramethyl-1,3,2-dioxaborolan-2-yl)-3-(trifluoromethyl)pyrazol-1-yl]acetonitrile. MS(m/e): 462.1 (M+H).

Step 3:

### 2-[4-[8-[3-chloro-4-[4-hydroxy-4-(methylaminomethyl)piperidine-1-carbonyl]anilino]imidazo[1,2-a]pyrazin-3-yl]-3-(trifluoromethyl)pyrazol-1-yl]acetonitrile

In analogy to the procedure described for example 1, step 3 the title compound was prepared from 2-chloro-4-[[3-[1-(cyanomethyl)-3-(trifluoromethyl)pyrazol-4-yl]imidazo[1,2-a]pyrazin-8-yl]amino]benzoic acid and tert-butyl N-[(4-hydroxy-4-piperidyl)methyl]-N-methyl-carbamate. MS(m/e): 688.3 (M+H).

Step 4:

### 2-[4-[8-[3-chloro-4-[4-hydroxy-4-(methylaminomethyl)piperidine-1-carbonyl]anilino]imidazo[1,2-a]pyrazin-3-yl]-3-(trifluoromethyl)pyrazol-1-yl]acetonitrile formate

To a solution of tert-butyl N-[[1-[2-chloro-4-[[3-[1-(cyanomethyl)-3-(trifluoromethyl)pyrazol-4-yl]imidazo[1,2-a]pyrazin-8-yl]amino]benzoyl]-4-hydroxy-4-piperidyl]methyl]-N-methyl-carbamate (25.0 mg, 0.040 mmol) in DCM (4.9 mL) was added 2,6-lutidine (0.01 mL, 0.070 mmol) and trimethylsilyl trifluormethanesulfonate (20.19 mg, 0.090 mmol) at 0° C., and the mixture was stirred at 25° C. for 0.5 h. The mixture of reaction was adjusted by NH₃·H₂O(aq) to pH=7 and evaporated. The residue was purified by preparative HPLC to yield after evaporation of the product containing fractions the title compound as off white solid acid (12.8 mg, 55.6%). MS(m/e): 588.1 (M+H).

### Example 15

### 2-ethyl-N-methyl-4-[[3-[1-(1H-pyrazol-3-ylmethyl)-3-(trifluoromethyl)pyrazol-4-yl]imidazo[1,2-a]pyrazin-8-yl]amino]benzamide

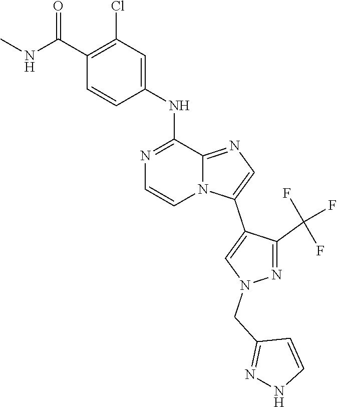 [SMILES: CNC(=O)c1ccc(Nc2nccn3c(-c4cn(Cc5cc[nH]n5)nc4C(F)(F)F)cnc23)cc1Cl]

Step 1:

### 2-ethyl-4-[(3-iodoimidazo[1,2-a]pyrazin-8-yl)amino]-N-methyl-benzamide

In analogy to the procedure described for the synthesis of example 1, step 3 the title compounds was prepared from 2-ethyl-4-[(3-iodoimidazo[1,2-a]pyrazin-8-yl)amino]benzoic acid, hydrochloride (example 1, step 2) and methyl amine as off-white solid. MS(m/e): 422.1 (M+H).

Step 2:

### 2-ethyl-N-methyl-4-[[3-[3-(trifluoromethyl)-1H-pyrazol-4-yl]imidazo[1,2-a]pyrazin-8-yl]amino]benzamide

In analogy to the procedure described for example 1, step 4 the title compound was prepared from 2-chloro-4-[(3-iodoimidazo[1,2-a]pyrazin-8-yl)amino]benzoic acid and (3-(trifluoromethyl)-1H-pyrazol-4-yl)boronic acid [CAS #1202054-12-3] as brown solid. MS(m/e): 430.3 (M+H).

Step 3:

### 2-ethyl-N-methyl-4-[[3-[1-(1H-pyrazol-3-ylmethyl)-3-(trifluoromethyl)pyrazol-4-yl]imidazo[1,2-a]pyrazin-8-yl]amino]benzamide

A mixture of 2-ethyl-N-methyl-4-((3-(3-(trifluoromethyl)-1H-pyrazol-4-yl)imidazo[1,2-a]pyrazin-8-yl)amino)benzamide (50 mg, 116 μmol), (1H-pyrazol-3-yl)methanol [CAS #23585-49-1] (13.7 mg, 140 μmol), tributylphosphine [CAS #998-40-3] (70.7 mg, 86 μl, 349 μmol) and ADDP [CAS #10465-81-3] (88.1 mg, 349 μmol) in DMF (1 mL) was stirred at room temperature for 1 h. The mixture was extracted with ethyl acetate and water (pH 8-9). The organic layer was dried with magnesium sulfate and the solvent was evaporated. The crude material was purified by flash chromatography on sorbent (silica gel, 20 g, 0% to 50% DCM/MeOH/NH₄OH (95/5/1)). The mixed fractions (50 mg) were purified by reverse phase under basic conditions to yield the title compound as white solid (20 mg, 34%). MS(m/e): 510.4 (M+H).

### Example 16

### N-[2-(2-aminoethoxy)ethyl]-2-methyl-4-[[3-[1-prop-2-ynyl-3-(trifluoromethyl)pyrazol-4-yl]imidazo[1,2-a]pyrazin-8-yl]amino]benzamide

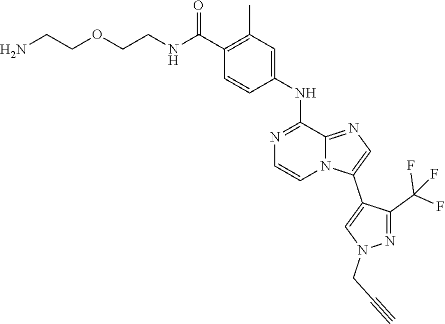 [SMILES: C#CCn1cc(-c2cnc3c(Nc4ccc(C(=O)NCCOCCN)c(C)c4)nccn23)c(C(F)(F)F)n1]

Step 1:

### 4-[(3-iodoimidazo[1,2-a]pyrazin-8-yl)amino]-2-methyl-benzoic acid

In analogy to the procedure described for example 1, step 1 the title compound was prepared from -chloro-3-iodoimidazo[1,2-a]pyrazine and 4-amino-2-methylbenzoic acid as white solid. MS(m/e): 395.1 (M+H).

Step 2:

### tert-butyl N-[2-[2-[[4-[(3-iodoimidazo[1,2-a]pyrazin-8-yl)amino]-2-methyl-benzoyl]amino]ethoxy]ethyl]carbamate

In analogy to the procedure described for example 1, step 3 the title compound was prepared from −4-[(3-iodoimidazo[1,2-a]pyrazin-8-yl)amino]-2-methyl-benzoic acid and tert-butyl (2-(2-aminoethoxy)ethyl)carbamate. MS(m/e): 581.3 (M+H).

Step 3:

### tert-butyl N-[2-[2-[[2-methyl-4-[[3-[3-(trifluoromethyl)-1H-pyrazol-4-yl]imidazo[1,2-a]pyrazin-8-yl]amino]benzoyl]amino]ethoxy]ethyl]carbamate

In analogy to the procedure described for example 1, step 4 the title compound was prepared from tert-butyl N-[2-[2-[[4-[(3-iodoimidazo[1,2-a]pyrazin-8-yl)amino]-2-methyl-benzoyl]amino]ethoxy]ethyl]carbamate and (3-(trifluoromethyl)-1H-pyrazol-4-yl)boronic acid. MS(m/e): 589.4 (M+H).

Step 4:

### N-[2-(2-aminoethoxy)ethyl]-2-methyl-4-[[3-[1-prop-2-ynyl-3-(trifluoromethyl)pyrazol-4-yl]imidazo[1,2-a]pyrazin-8-yl]amino]benzamide

In analogy to the procedure described for example 1, step 5 the title compound was prepared from tert-butyl N-[2-[2-[[2-methyl-4-[[3-[3-(trifluoromethyl)-1H-pyrazol-4-yl]imidazo[1,2-a]pyrazin-8-yl]amino]benzoyl]amino]ethoxy]ethyl]carbamate and 3-bromoprop-1-yne followed by acidic Boc deprotection. MS(m/e): 527.5 (M+H).

### Example 17

### [4-(3-aminocyclobutanecarbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone formate

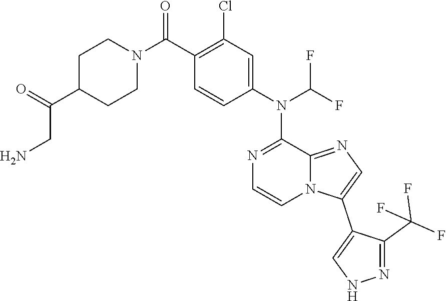 [SMILES: NCC(=O)C1CCN(C(=O)c2ccc(N(c3nccn4c(-c5c[nH]nc5C(F)(F)F)cnc34)C(F)F)cc2Cl)CC1]

Step 1:

### tert-butyl 4-[4-[(3-iodoimidazo[1,2-a]pyrazin-8-yl)amino]-2-methyl-benzoyl]piperazine-1-carboxylate

In analogy to the procedure described for example 1, step 3 the title compound was prepared from −4-[(3-iodoimidazo[1,2-a]pyrazin-8-yl)amino]-2-methyl-benzoic acid (example 16, step 1) and Boc-piperazine. MS(m/e): 563.0 (M+H).

Step 2:

### tert-butyl 4-[2-methyl-4-[[3-[3-(trifluoromethyl)-1H-pyrazol-4-yl]imidazo[1,2-a]pyrazin-8-yl]amino]benzoyl]piperazine-1-carboxylate

In analogy to the procedure described for example 1, step 4 the title compound was prepared from tert-butyl 4-[4-[(3-iodoimidazo[1,2-a]pyrazin-8-yl)amino]-2-methyl-benzoyl]piperazine-1-carboxylate and (3-(trifluoromethyl)-1H-pyrazol-4-yl)boronic acid. MS(m/e): 571.3 (M+H).

Step 3:

### [2-methyl-4-[[3-[3-(trifluoromethyl)-1H-pyrazol-4-yl]imidazo[1,2-a]pyrazin-8-yl]amino]phenyl]-piperazin-1-yl-methanone

In analogy to the procedure described for example 1, step 5 the title compound was prepared from tert-butyl 4-[2-methyl-4-[[3-[3-(trifluoromethyl)-1H-pyrazol-4-yl]imidazo[1,2-a]pyrazin-8-yl]amino]benzoyl]piperazine-1-carboxylate by acidic Boc deprotection. MS(m/e): 471.3 (M+H).

Step 4:

### tert-butyl N-[2-[4-[2-methyl-4-[[3-[3-(trifluoromethyl)-1H-pyrazol-4-yl]imidazo[1,2-a]pyrazin-8-yl]amino]benzoyl]piperazin-1-yl]-2-oxo-ethyl]carbamate

In analogy to the procedure described for example 5, step 3 the title compound was prepared from [2-methyl-4-[[3-[3-(trifluoromethyl)-1H-pyrazol-4-yl]imidazo[1,2-a]pyrazin-8-yl]amino]phenyl]-piperazin-1-yl-methanone and (tert-butoxycarbonyl)glycine. MS(m/e): 628.5 (M+H).

Step 5:

### [4-(3-aminocyclobutanecarbonyl)piperazin-1-yl]-[2-chloro-4-[[3-[3-(trifluoromethyl)-1H-pyrazol-4-yl]imidazo[1,2-a]pyrazin-8-yl]amino]phenyl]methanone formate

In analogy to the procedure described for example 1, step 5 the title compound was prepared from tert-butyl N-[2-[4-[2-methyl-4-[[3-[3-(trifluoromethyl)-1H-pyrazol-4-yl]imidazo[1,2-a]pyrazin-8-yl]amino]benzoyl]piperazin-1-yl]-2-oxo-ethyl]carbamate by acidic Boc deprotection. MS(m/e): 528.4 (M+H).

### Example 18

### N-[2-(2-aminoethoxy)ethyl]-4-[[3-[1-(cyanomethyl)-3-(difluoromethyl)pyrazol-4-yl]imidazo[1,2-a]pyrazin-8-yl]amino]-2-ethylbenzamide

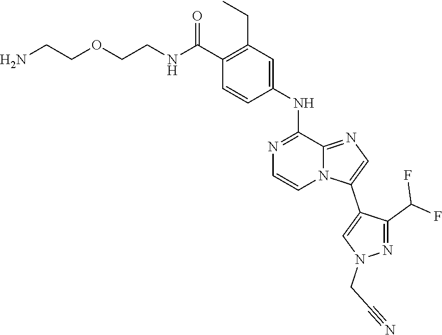 [SMILES: CCc1cc(Nc2nccn3c(-c4cn(CC#N)nc4C(F)F)cnc23)ccc1C(=O)NCCOCCN]

Step 1:

### tert-butyl N-[2-[2-[[4-[[3-[3-(difluoromethyl)-1H-pyrazol-4-yl]imidazo[1,2-a]pyrazin-8-yl]amino]-2-ethyl-benzoyl]amino]ethoxy]ethyl]carbamate

In analogy to the procedure described for example 1, step 4 the title compound was prepared from tert-butyl N-[2-[2-[[2-ethyl-4-[(3-iodoimidazo[1,2-a]pyrazin-8-yl)amino]benzoyl]amino]ethoxy] ethyl] carbamate (example 1, step 3) and 3-(difluoromethyl)-4-(4,4,5,5-tetramethyl-1,3,2-dioxaborolan-2-yl)-1H-pyrazole. MS(m/e): 585.4 (M+H).

Step 2:

### tert-butyl N-[2-[2-[[4-[[3-[1-(cyanomethyl)-3-(difluoromethyl)pyrazol-4-yl]imidazo[1,2-a]pyrazin-8-yl]amino]-2-ethyl-benzoyl]amino]ethoxy]ethyl]carbamate

In analogy to the procedure described for example 2, step 2 the title compound was prepared from tert-butyl N-[2-[2-[[4-[[3-[3-(difluoromethyl)-1H-pyrazol-4-yl]imidazo[1,2-a]pyrazin-8-yl]amino]-2-ethyl-benzoyl]amino]ethoxy]ethyl]carbamate and 2-bromoacetonitrile [CAS #590-17-0]. MS(m/e): 624.4 (M+H).

Step 3:

### N-[2-(2-aminoethoxy)ethyl]-4-[[3-[1-(cyanomethyl)-3-(difluoromethyl)pyrazol-4-yl]imidazo[1,2-a]pyrazin-8-yl]amino]-2-ethylbenzamide

In analogy to the procedure described for example 1, step 5 the title compound was prepared from tert-butyl N-[2-[2-[[4-[[3-[1-(cyanomethyl)-3-(difluoromethyl)pyrazol-4-yl]imidazo[1,2-a]pyrazin-8-yl]amino]-2-ethyl-benzoyl]amino]ethoxy]ethyl]carbamate by acidic Boc deprotection. MS(m/e): 524.4 (M+H).

### Example 19

### 2-[4-[4-[[3-[1-(cyanomethyl)-3-(trifluoromethyl)pyrazol-4-yl]imidazo[1,2-a]pyrazin-8-yl]amino]-2-methylbenzoyl]piperazin-1-yl]ethyl-trimethylazanium formate

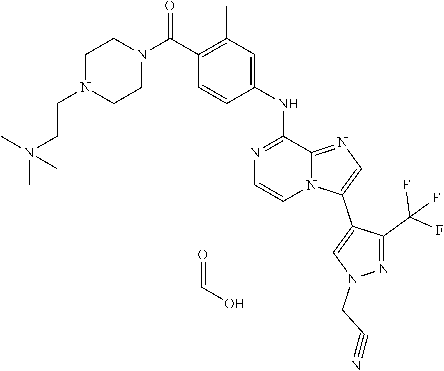

Step 1:

### 4-[[3-[1-(cyanomethyl)-3-(trifluoromethyl)pyrazol-4-yl]imidazo[1,2-a]pyrazin-8-yl]amino]-2-methyl-benzoic acid

In analogy to the procedure described for example 1, step 4 the title compound was prepared from 4-[(3-iodoimidazo[1,2-a]pyrazin-8-yl)amino]-2-methyl-benzoic acid (example 16, step 1) and (3-(trifluoromethyl)-1H-pyrazol-4-yl)boronic acid. MS(m/e): 442.0 (M+H).

Step 2:

### 2-[4-[4-[[3-[1-(cyanomethyl)-3-(trifluoromethyl)pyrazol-4-yl]imidazo[1,2-a]pyrazin-8-yl]amino]-2-methylbenzoyl]piperazin-1-yl]ethyl-trimethylazanium formate

In analogy to the procedure described for example 1, step 3 the title compound was prepared from 4-[[3-[1-(cyanomethyl)-3-(trifluoromethyl)pyrazol-4-yl]imidazo[1,2-a]pyrazin-8-yl]amino]-2-methyl-benzoic acid and trimethyl(2-piperazin-1-ylethyl)ammonium chloride. MS(m/e): 595.2 (M+H).

### Example 20

### N-[2-(2-aminoethoxy)ethyl]-4-[[3-[3-(difluoromethyl)-1-(methoxymethyl)pyrazol-4-yl]imidazo[1,2-a]pyrazin-8-yl]amino]-2-ethylbenzamide

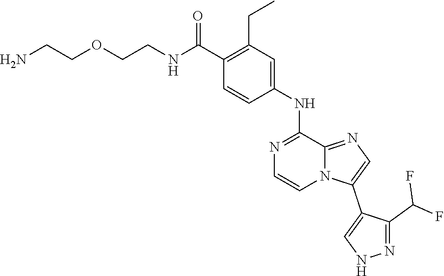 [SMILES: CCc1cc(Nc2nccn3c(-c4c[nH]nc4C(F)F)cnc23)ccc1C(=O)NCCOCCN]

Step 1:

### 4-bromo-3-(difluoromethyl)-1-(methoxymethyl)pyrazole

To a solution of 4-bromo-3-(difluoromethyl)-1H-pyrazole (250 mg, 1.27 mmol, CAS 1451392-65-6) and potassium carbonate (351 mg, 2.54 mmol) in DMF (8 mL) was added iodo(methoxy)methane (262 mg, 129 μl, 1.52 mmol) and stirred at 22° C. under argon overnight. The reaction mixture was poured into water (50 mL) and extracted with ethyl acetate (2×50 mL). The organic layers were combined, adsorbed on Isolute and purified by flash chromatography (silica gel, 12 g, 0% to 60% TBME) to yield after evaporation of the product containing fractions the title compound as colorless liquid (142 mg, 46%). MS(m/e): 243.0 (M+H).

Step 2:

3-(difluoromethyl)-1-(methoxymethyl)-4-(4,4,5,5-tetramethyl-1,3,2-dioxaborolan-2-yl)pyrazole 4-bromo-3-(difluoromethyl)-1-(methoxymethyl)-1H-pyrazole (142 mg, 589 μmol) was dissolved under argon in dry THF (15 mL). The mixture was cooled to −78° C. N-butyllithium (442 μl, 707 μmol) was added dropwise and the mixture was stirred at −78° C. for 40 min. 2-isopropoxy-4,4,5,5-tetramethyl-1,3,2-dioxaborolane (164 mg, 180 μl, 884 μmol) was added dropwise. After reaction completion the mixture was poured into NH₄Cl sat. (25 mL) and extracted with ethyl acetate (2×15 mL). The crude material was adsorbed on Isolute and purified by flash chromatography (silica gel, SiliCycle 10 g cartridge (40-63 μm), 0% to 100% MTBE in heptane).Two regioisomers were formed 3-(difluoromethyl)-1-(methoxymethyl)-4-(4,4,5,5-tetramethyl-1,3,2-dioxaborolan-2-yl)-1H-pyrazole and 3-(difluoromethyl)-1-(methoxymethyl)-5-(4,4,5,5-tetramethyl-1,3,2-dioxaborolan-2-yl)-1H-pyrazole (72.7 mg, 21%) and used in the subsequent step without further purification. MS(m/e): 289.3 (M+H).

Step 3:

### tert-butyl N-[2-[2-[[4-[[3-[3-(difluoromethyl)-1-(methoxymethyl)pyrazol-4-yl]imidazo[1,2-a]pyrazin-8-yl]amino]-2-ethyl-benzoyl]amino]ethoxy]ethyl]carbamate

In analogy to the procedure described for example 1, step 4 the title compound was prepared from tert-butyl N-[2-[2-[[2-ethyl-4-[(3-iodoimidazo[1,2-a]pyrazin-8-yl)amino]benzoyl]amino]ethoxy] ethyl] carbamate (example 1, step 3) and 3-(difluoromethyl)-1-(methoxymethyl)-4-(4,4,5,5-tetramethyl-1,3,2-dioxaborolan-2-yl)pyrazole (regioisomeric mixture from example 20, step 2). MS(m/e): 629.3 (M+H).

Step 4:

### 2-[4-[4-[[3-[1-(cyanomethyl)-3-(trifluoromethyl)pyrazol-4-yl]imidazo[1,2-a]pyrazin-8-yl]amino]-2-methylbenzoyl]piperazin-1-yl]ethyl-trimethylazanium formate

In analogy to the procedure described for example 1, step 5 the title compound was prepared from tert-butyl N-[2-[2-[[4-[[3-[3-(difluoromethyl)-1-(methoxymethyl)pyrazol-4-yl]imidazo[1,2-a]pyrazin-8-yl]amino]-2-ethyl-benzoyl]amino]ethoxy]ethyl]carbamate by acidic Boc deprotection. MS(m/e): 529.3 (M+H).

The following examples were synthesized in analogy to procedures described before. The reaction sequence and staring materials are recorded in the Table 1.

TABLE 1

| | | | | MW | |
|---|---|---|---|---|---|
| | | | Parent | (M + H) | Reaction Sequence and Starting |
| No | Structure | Systematic Name | MW | found | Materials |
| 21 | 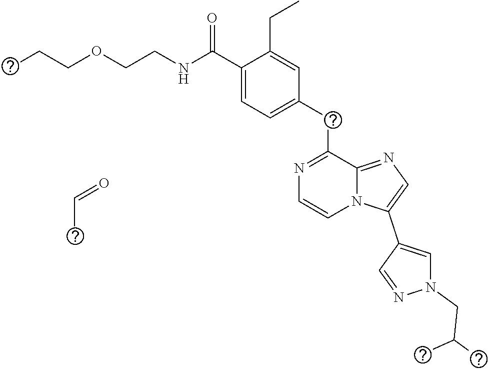 | N-[2-(2- aminoethoxy)ethyl]- 4-[[3-[1-(2,2- difluoroethyl) pyrazol-4- yl]imidazo[1,2- a]pyrazin-8- yl]amino]-2- ethylbenzamide formate | 498.5 | 497.4 (M − H)− | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and 1-(2,2-difluoroethyl)-4- (4,4,5,5-tetramethyl-1,3,2- dioxaborolan-2-yl)-1H-pyrazole with subsequent acidic cleavage of the Boc group |
| 22 | 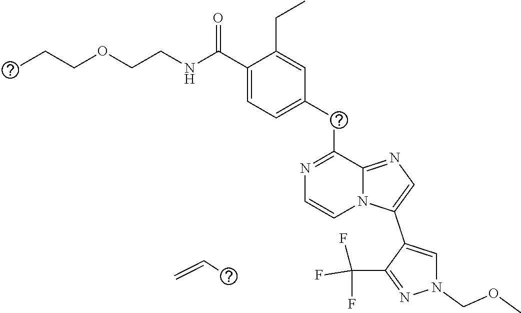 | N-[2-(2- aminoethoxy)ethyl]- 2-ethyl-4-[[3- [1- (methoxymethyl)- 3- (trifluoromeethyl) pyrazol-4- yl]imidazo[1,2- a]pyrazin-8- yl]amino] benzamide formate | 502.5 | 503.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and 1-(methoxymethyl)-3- (trifluoromethyl)-1H-pyrazol-4- yl)boronic acid with subsequent acidic cleavage of the Boc group |
| 23 | 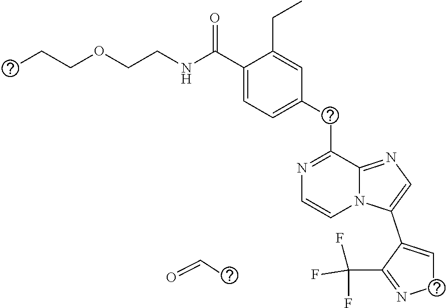 | N-[2-(2- aminoethoxy)ethyl]- 2-ethyl-4-[[3- [3- (trifluoromeethyl) 1H-pyrazol-4- yl]imidazo[1,2- a]pyrazin-8- yl]amino] benzamide formate | 502.5 | 503.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and 1-(methoxymethyl)-3- (trifluoromethyl)-1H-pyrazol-4- yl)boronic acid with subsequent acidic cleavage of the Boc and MOM group |
| 24 | 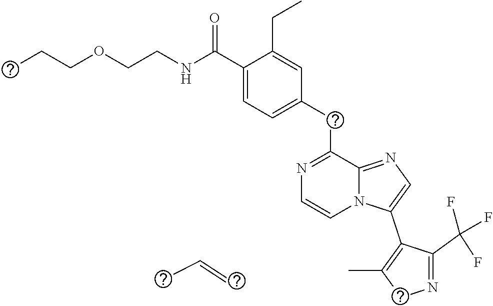 | N-[2-(2- aminoethoxy)ethyl]- 2-ethyl-4-[[3- [5-methyl-3- (trifluoromethyl)- 1H-pyrazol-4- yl]imidazo[1,2- a]pyrazin-8- yl]amino] benzamide formate | 516.5 | 517.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and 5-methyl-4-(4,4,5,5- tetramethyl-1,3,2-dioxaborolan-2- yl)-3-(trifluoromethyl)-1H- pyrazole with subsequent acidic cleavage of the Boc group |
| 25 | 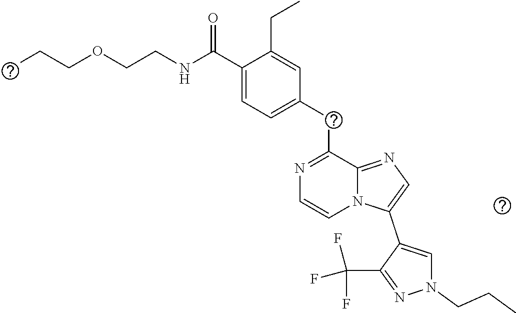 [SMILES: CCCOCCNC(=O)c1ccc(Cc2nccn3c(-c4cn(CCC)nc4C(F)(F)F)cnc23)cc1CC] | N-[2-(2- aminoethoxy) ethyl]-2-ethyl-4-[[3- [1-propyl-3- (trifluoromethyl) pyrazol-4- yl]imidazo[1,2- a]pyrazin-8- yl]amino]benzamide hydrochloride | 544.6 | 545.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and (1-propyl-3- (trifluoromethyl)-1H-pyrazol-4- yl)boronic acid with subsequent acidic cleavage of the Boc group |
| 26 | 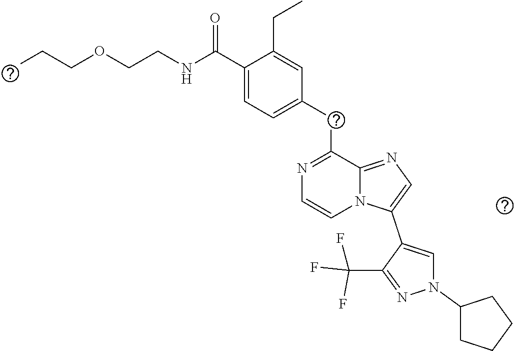 [SMILES: CCCOCCNC(=O)c1ccc(Cc2nccn3c(-c4cn(C5CCCC5)nc4C(F)(F)F)cnc23)cc1CC] | N-[2-(2- aminoethoxy)ethyl]- 4-[[3-[1- cyclopentyl-3- (trifluoromethyl) pyrazol-4- yl]imidazo[1,2- a]pyrazin-8- yl]amino]-2- ethylbenzamide hydrochloride | 570.6 | 571.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and (1-cyclopentyl-3- (trifluoromethyl)-1H-pyrazol-4- yl)boronic acid with subsequent acidic cleavage of the Boc group |
| 27 | 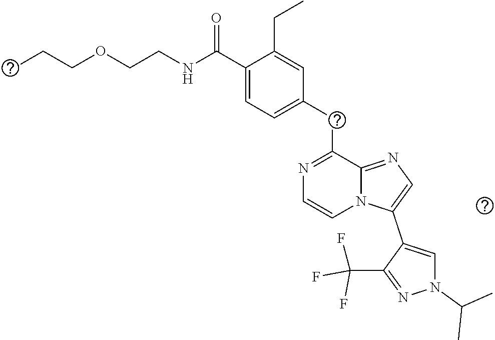 [SMILES: CCCOCCNC(=O)c1ccc(Cc2nccn3c(-c4cn(C(C)C)nc4C(F)(F)F)cnc23)cc1CC] | N-[2-(2- aminoethoxy)ethyl]- 2-ethyl-4-[[3- [1-propan-2-yl-3- (trifluoromethyl) pyrazol-4- yl]imidazo[1,2- a]pyrazin-8- yl]amino]benzamide hydrochloride | 544.6 | 545.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and (1-isopropyl-3- (trifluoromethyl)-1H-pyrazol-4- yl)boronic acid with subsequent acidic cleavage of the Boc group |
| 28 | 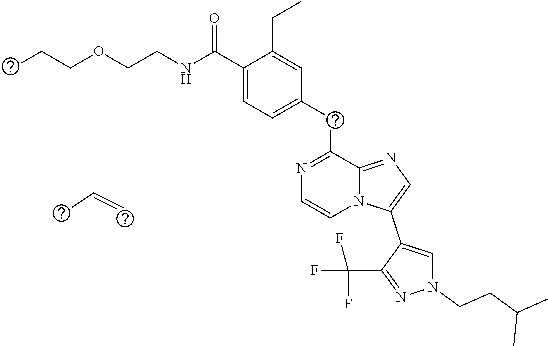 | N-[2-(2- aminoethoxy)ethyl]- 2-ethyl-4-[[3- [1-(3- methylbutyl)-3- (trifluoromethyl) pyrazol-4- yl]imidazo[1,2- a]pyrazin-8- yl]amino]benzamide formate | 572.6 | 573.3 | in analogy to the procedure for example 1: from tert-butyl N-[2- [2-[[2-ethyl-4-[(3- iodoimidazo[1,2-a]pyrazin-8- yl)amino]benzoyl]amino]ethoxy] ethyl]carbamate (example 1, step 3) and (1-isopentyl-3- (trifluoromethyl)-1H-pyrazol-4- yl)boronic acid with subsequent acidic cleavage of the Boc group |
| 29 | 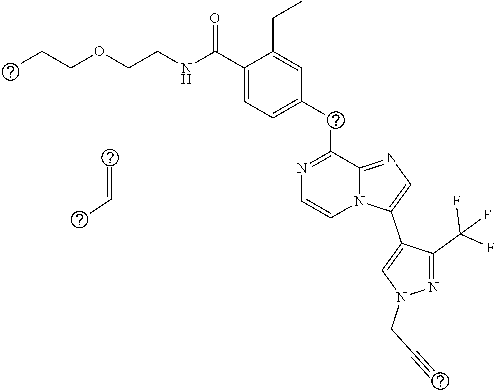 | N-[2-(2- aminoethoxy)ethyl]- 4-[[3-[1- (cyanomethyl)-3- (trifluoromethyl) pyrazol-4- yl]imidazo[1,2- a]pyrazin-8- yl]amino]-2- ethylbenzamide formate | 541.5 | 542.3 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) and 2-iodoacetonitrile with subsequent acidic cleavage of the Boc group |
| 30 | 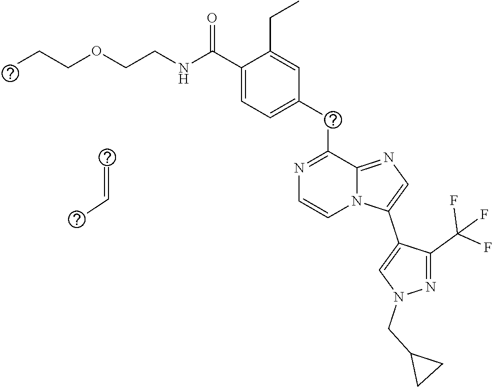 | N-[2-(2- aminoethoxy)ethyl]- 4-[[3-[1- (cyclopropylmethyl)- 3- (trifluoromethyl) pyrazol-4- yl]imidazo[1,2- a]pyrazin-8- yl]amino]-2- ethylbenzmamide formate | 556.6 | 557.3 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) and (bromomethyl)cyclopropane with subsequent acidic cleavage of the Boc group |
| 31 | 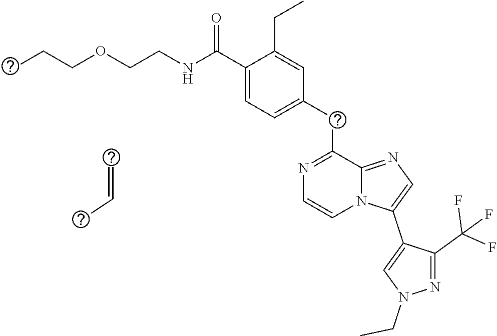 | N-[2-(2- aminoethoxy)ethyl]- 2-ethyl-4-[[3- [1-ethyl-3- (trifluoromethyl) pyrazol-4- yl]imidazo[1,2- a]pyrazin-8- yl]amino]benzamide formate | 530.5 | 531.3 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) and iodoethane with subsequent acidic cleavage of the Boc group |
| 32 | 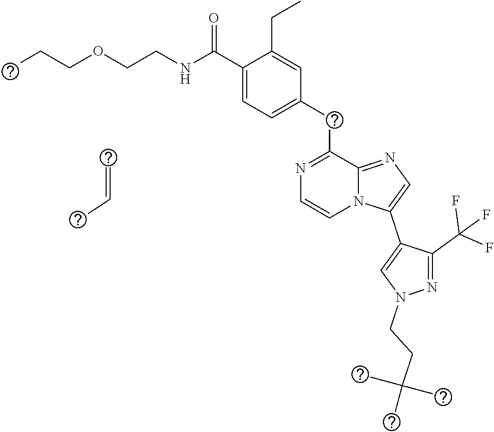 | N-[2-(2- aminoethoxy)ethyl]- 2-ethyl-4-[[3- [3- (trifluoromethyl)- 1-(3,3,3- (trifluoropropyl) pyrazol-4- yl]imidazo[1,2- a]pyrazin-8- yl]amino]benzamide formate | 598.5 | 599.2 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) and 1,1,1-trifluoro-3-iodopropane with subsequent acidic cleavage of the Boc group |
| 33 | 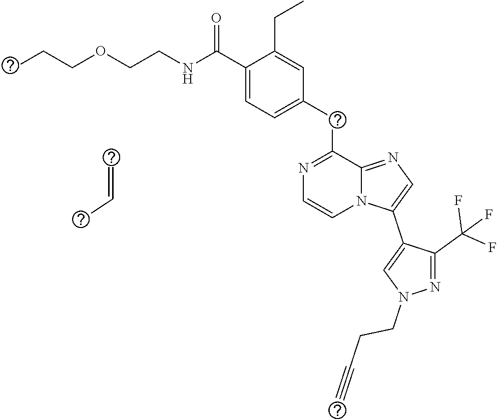 | N-[2-(2- aminoethoxy)ethyl]- 4-[[3-[1-(2- cyanoethyl)-3- (trifluoromethyl) pyrazol-4- yl]imidazo[1,2- a]pyrazin-8- yl]amino]-2- ethylbenzamide formate | 555.6 | 556.3 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) and 3-bromopropanenitrile with subsequent acidic cleavage of the Boc group |
| 34 | 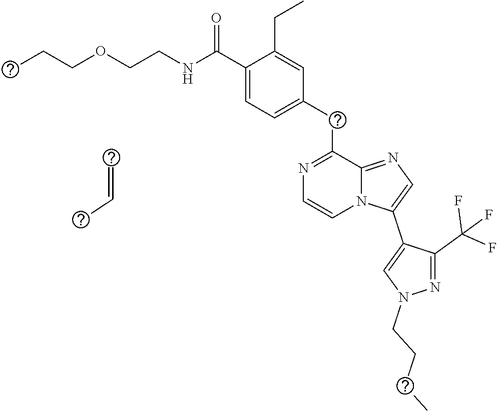 | N-[2-(2- aminoethoxy)ethyl]- 2-ethyl-4-[[3- [1-(2- methoxyethyl)-3- (trifluoromethyl) pyrazol-4- yl]imidazo[1,2- a]pyrazin-8- yl]amino]benzamide formate | 560.6 | 561.3 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) and 1-iodo-2-methoxyethane with subsequent acidic cleavage of the Boc group |
| 35 | 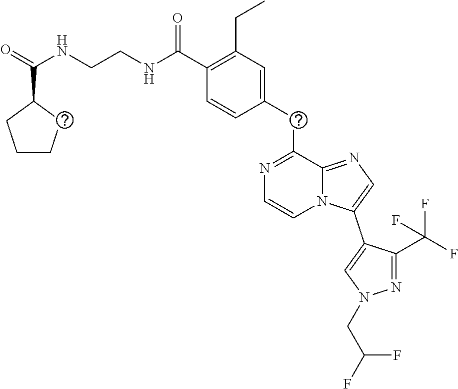 | (2S)-N-[2-[[4-[[3- [1-(2,2- difluoroethyl)-3- (trifluoromethyl) pyrazol-4- yl]imidazo[1,2- a]pyrazin-8- yl]amino]-2- ethylbenzoyl)amino] ethyl]pyrrolidine- 2-carboxamide | 619.6 | 620.4 | in analogy to the procedure for example 3: from tert-butyl (2S)-2- [2-[[2-ethyl-4-[[3-[3- (trifluoromethyl)-1H-pyrazol-4- yl]imidazo[1,2-a]pyrazin-8- yl]amino]benzoyl]amino]ethyl carbamoyl]pyrrolidine-1-carboxylate and 1,1-difluoro-2-iodoethane CAS [598-39-0] with subsequent acidic cleavage of the Boc group |
| 36 | 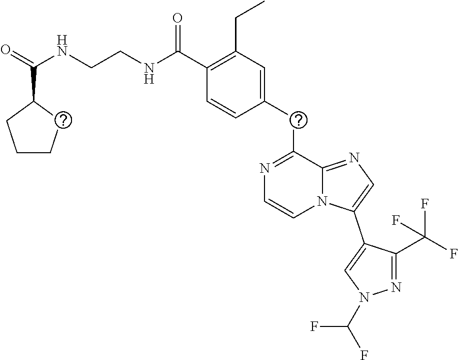 | (2S)-N-[2-[[4-[[3- [1- (difluoromethyl)- 3- (trifluoromethyl) pyrazol-4- yl]imidazo[1,2- a]pyrazin-8- yl]amino]-2- ethylbenzoyl) amino]ethyl] pyrrolidine-2- carboxamide | 605.6 | 606.4 | in analogy to the procedure for example 3: from tert-butyl (2S)-2- [2-[[2-ethyl-4-[[3-[3- (trifluoromethyl)-1H-pyrazol-4- yl]imidazo[1,2-a]pyrazin-8- yl]amino]benzoyl]amino]ethyl carbamoyl]pyrrolidine-1-carboxylate and methyl 2-chloro-2,2- difluoroacetate with subsequent acidic cleavage of the Boc group |
| 37 | 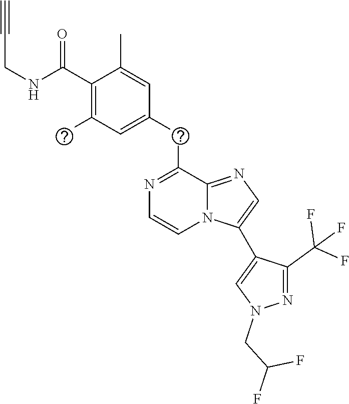 [SMILES: C#CCNC(=O)c1c(C)cc(Cc2nccn3c(-c4cn(CC(F)F)nc4C(F)(F)F)cnc23)cc1C] | 4-[[3-[1-(2,2- difluoroethyl)-3- (trifluoromethyl) pyrazol-4- yl]imidazo[1,2- a]pyrazin-8- yl]amino]-2- fluoro-6-methyl- N-prop-2- ynylbenzamide | 521.4 | 522.2 | in analogy to the procedure for example 4: from 2-fluoro-6- methyl-N-prop-2-ynyl-4-[[3-[3- (trifluoromethyl)-1H-pyrazol-4- yl]imidazo[1,2-a]pyrazin-8- yl]amino]benzamide and 1,1- difluoro-2-iodo-ethane |
| 38 | 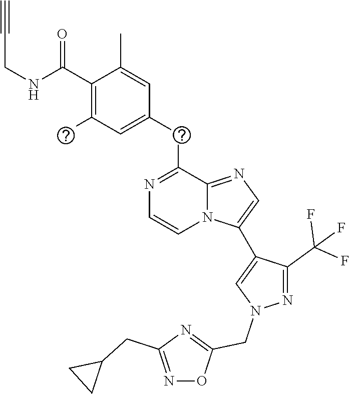 [SMILES: C#CCNC(=O)c1c(C)cc(Cc2nccn3c(-c4cn(Cc5nc(CC6CC6)no5)nc4C(F)(F)F)cnc23)cc1C] | 4-[[3-[1-[[3- (cyclopropylmethyl)- 1,2,4- oxadiazol-5- yl]methyl]-3- (trifluoromethyl) pyrazol-4- yl]imidazo[1,2- a]pyrazin-8- yl]amino]-2- fluoro-6-methyl- N-prop-2- ynylbenzamide | 593.5 | 594.3 | in analogy to the procedure for example 4: from 2-fluoro-6- methyl-N-prop-2-ynyl-4-[[3-[3- (trifluoromethyl)-1H-pyrazol-4- yl]imidazo[1,2-a]pyrazin-8- yl]amino]benzamide and 5- (chloromethyl)-3- (cyclopropylmethyl)-1,2,4- oxadiazole |
| 39 | 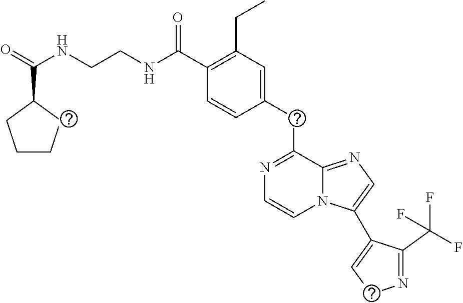 | (2S)-N-[2-[[2- ethyl-4-[[3-[3- (trifluoromethyl)- 1H-pyrazol-4- yl]imidazo[1,2- a]pyrazin-8- yl]amino]benzoyl] amino]ethyl] pyrrolidine-2- carboxamide | 555.6 | 556.4 | in analogy to the procedure for example 3: from tert-butyl (2S)-2- [2-[[2-ethyl-4-[[3-[3- (trifluoromethyl)-1H-pyrazol-4- yl]imidazo[1,2-a]pyrazin-8- yl]amino]benzoyl]amino]ethyl carbamoyl]pyrrolidine-1-carboxylate with subsequent acidic cleavage of the Boc group |
| 40 | 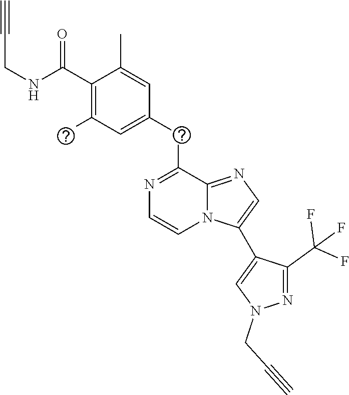 [SMILES: C#CCNC(=O)c1c(C)cc(Cc2nccn3c(-c4cn(CC#C)nc4C(F)(F)F)cnc23)cc1C] | 2-fluoro-6- methyl-N-prop-2- ynyl-4-[[3-[1- prop-2-ynyl-3- (trifluoromethyl) pyrazol-4- yl]imidazo[1,2- a]pyrazin-8- yl]amino] benzamide | 495.4 | 496.2 | in analogy to the procedure for example 4: from 2-fluoro-6- methyl-N-prop-2-ynyl-4-[[3-[3- (trifluoromethyl)-1H-pyrazol-4- yl]imidazo[1,2-a]pyrazin-8- yl]amino]benzamide and 3- bromoprop-1-yne |
| 41 | 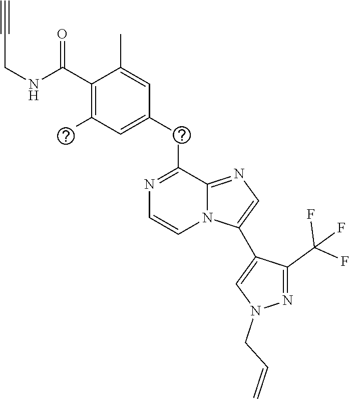 [SMILES: C#CCNC(=O)c1c(C)cc(Cc2nccn3c(-c4cn(CC=C)nc4C(F)(F)F)cnc23)cc1C] | 2-fluoro-6- methyl-4-[[3-[1- prop-2-enyl-3- (trifluoromethyl) pyrazol-4- yl]imidazo[1,2- a]pyrazin-8- yl]amino]-N-prop- 2-ynylbenzamide | 497.5 | 498.2 | in analogy to the procedure for example 4: from 2-fluoro-6- methyl-N-prop-2-ynyl-4-[[3-[3- (trifluoromethyl)-1H-pyrazol-4- yl]imidazo[1,2-a]pyrazin-8- yl]amino]benzamide and 3- bromoprop-1-ene |
| 42 | 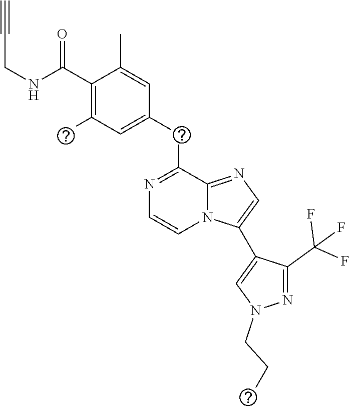 [SMILES: C#CCNC(=O)c1c(C)cc(Cc2nccn3c(-c4cn(CCC)nc4C(F)(F)F)cnc23)cc1C] | 2-fluoro-4-[[3-[1- (2-fluoroethyl)-3- (trifluoromethyl) pyrazol-4- yl]imidazo[1,2- a]pyrazin-8- yl]amino]-6- methyl-N-prop-2- ynylbenzamide | 503.4 | 504.2 | in analogy to the procedure for example 4: from 2-fluoro-6- methyl-N-prop-2-ynyl-4-[[3-[3- (trifluoromethyl)-1H-pyrazol-4- yl]imidazo[1,2-a]pyrazin-8- yl]amino]benzamide and 1-bromo- 2-fluoroethane |
| 43 | 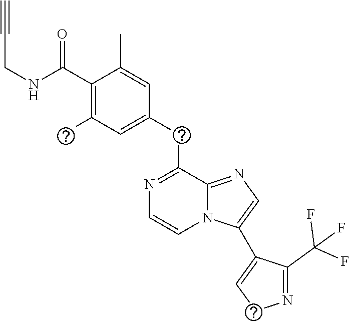 [SMILES: C#CCNC(=O)c1c(C)cc(Cc2nccn3c(C4=CCN=C4C(F)(F)F)cnc23)cc1C] | 2-fluoro-6- methyl-N-prop-2- ynyl-4-[[3-[3- (trifluoromethyl)- 1H-pyrazol-4- yl]imidazo[1,2- a]pyrazin-8- yl]amino] benzamide | 457.4 | 458.2 | example 4, step 4 |
| 44 | 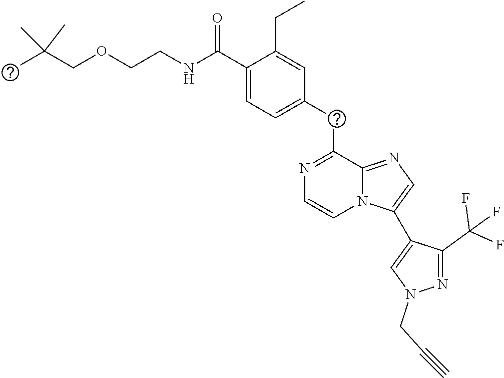 [SMILES: C#CCn1cc(-c2cnc3c(Cc4ccc(C(=O)NCCOCC(C)(C)C)c(CC)c4)nccn23)c(C(F)(F)F)n1] | N-[2-(2-amino-2- methylpropoxy) ethyl]-2-ethyl-4-[[3- [1-prop-2-ynyl-3- (trifluoromethyl) pyrazol-4- yl]imidazo[1,2- a]pyrazin-8- yl]amino]benzamide | 568.6 | 569.3 | in analogy to the procedures for example 1: amide coupling of 2- ethyl-4-[(3-iodoimidazo[1,2- a]pyrazin-8-yl)amino]benzoic acid, hydrochloride with tert-butyl (1- (2-aminoethoxy)-2-methylpropan- 2-yl)carbamate, subsequent Suzuki reaction wtih (3-(trifluoromethyl)- 1H-pyrazol-4-yl)boronic acid, alkylation with 3-bromoprop-1- yne and acidic Boc deprotection |
| 45 | 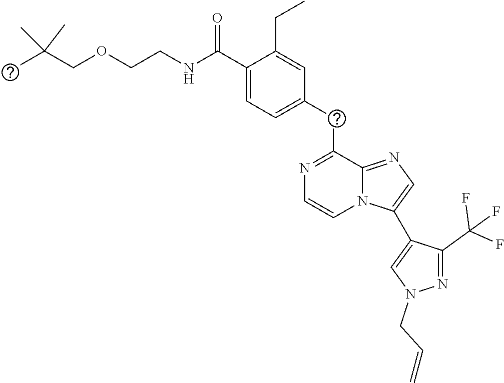 [SMILES: C=CCn1cc(-c2cnc3c(Cc4ccc(C(=O)NCCOCC(C)(C)C)c(CC)c4)nccn23)c(C(F)(F)F)n1] | N-[2-(2-amino-2- methylpropoxy) ethyl]-2-ethyl-4-[[3- [1-prop-2-enyl-3- (trifluoromethyl) pyrazol-4- yl]imidazo[1,2- a]pyrazin-8- yl]amino] benzamide | 570.6 | 571.4 | in analogy to the procedures for example 1: amide coupling of 2- ethyl-4-[(3-iodoimidazo[1,2- a]pyrazin-8-yl)amino]benzoic acid, hydrochloride with tert-butyl (1- (2-aminoethoxy)-2-methylpropan- 2-yl)carbamate, subsequent Suzuki reaction wtih (3-(trifluoromethyl)- 1H-pyrazol-4-yl)boronic acid, alkylation with 3-bromoprop-1-ene and acidic Boc deprotection |
| 46 | 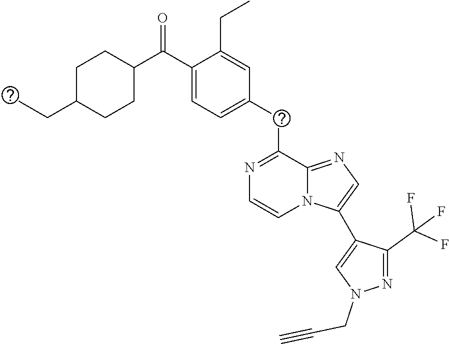 [SMILES: C#CCn1cc(-c2cnc3c(Cc4ccc(C(=O)C5CCC(CC)CC5)c(CC)c4)nccn23)c(C(F)(F)F)n1] | [4- (aminomethyl) piperidin-1-yl]-[2- ethyl-4-[[3-[1- prop-2-ynyl-3- (trifluoromethyl) pyrazol-4- yl]imidazo[1,2- a]pyrazin-8- yl]amino]phenyl] methanone | 550.6 | 551.3 | in analogy to the procedures for example 1: amide coupling of 2- ethyl-4-[(3-iodoimidazo[1,2- a]pyrazin-8-yl)amino]benzoic acid, hydrochloride with tert-butyl (piperidin-4-ylmethyl)carbamate, subsequent Suzuki reaction with (3-(trifluoromethyl)-1H-pyrazol-4- yl)boronic acid, alkylation with 3- bromoprop-1-yne and acidic Boc deprotection |
| 47 | 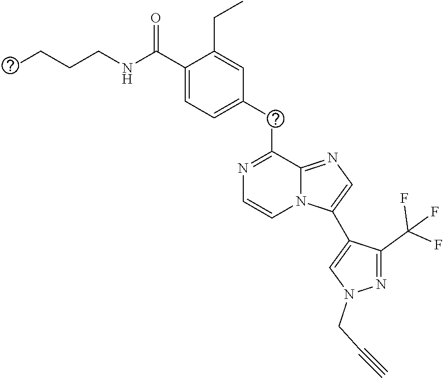 [SMILES: C#CCn1cc(-c2cnc3c(Cc4ccc(C(=O)NCCCC)c(CC)c4)nccn23)c(C(F)(F)F)n1] | N-(3- aminopropyl)-2- ethyl-4-[[3-[1- prop-2-ynyl-3- (trifluoromethyl) pyrazol-4- yl]imidazo[1,2- a]pyrazin-8- yl]amino] benzamide | 510.5 | 511.3 | in analogy to the procedures for example 1: amide coupling of 2- ethyl-4-[(3-iodoimidazo[1,2- a]pyrazin-8-yl)amino]benzoic acid, hydrochloride with tert-butyl (3- aminopropyl)carbamate, subsequent Suzuki reaction with (3-(trifluoromethyl)-1H-pyrazol-4- yl)boronic acid, alkylation with 3- bromoprop-1-yne and acidic Boc deprotection |
| 48 | 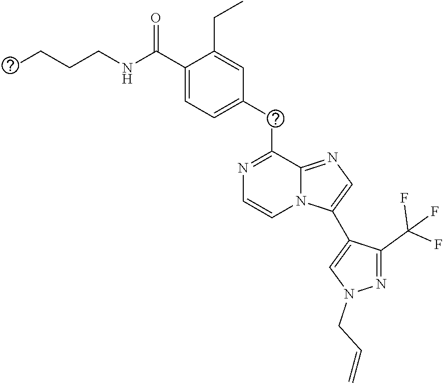 [SMILES: C=CCn1cc(-c2cnc3c(Cc4ccc(C(=O)NCCCC)c(CC)c4)nccn23)c(C(F)(F)F)n1] | N-(3- aminopropyl)-2- ethyl-4-[[3-[1- prop-2-enyl-3- (trifluoromethyl) pyrazol-4- yl]imidazo[1,2- a]pyrazin-8- yl]amino] benzamide | 512.5 | 513.3 | in analogy to the procedures for example 1: amide coupling of 2- ethyl-4-[(3-iodoimidazo[1,2- a]pyrazin-8-yl)amino]benzoic acid, hydrochloride with tert-butyl (3- aminopropyl)carbamate, subsequent Suzuki reaction with (3-(trifluoromethyl)-1H-pyrazol-4- yl)boronic acid, alkylation with 3- bromoprop-1-ene and acidic Boc deprotection |
| 49 | 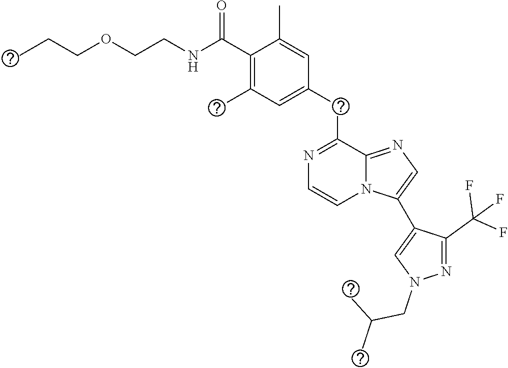 [SMILES: CCCOCCNC(=O)c1c(C)cc(Cc2nccn3c(-c4cn(CC(C)C)nc4C(F)(F)F)cnc23)cc1C] | N-[2-(2- aminoethoxy)ethyl]- 4-[[3-[1-(2,2- difluoroethyl)-3- (trifluoromethyl) pyrazol-4- yl]imidazo[1,2- a]pyrazin-8- yl]amino]-2- fluoro-6- methylbenzamide | 570.5 | 571.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (2-(2- aminoethoxy)ethyl)carbamate, subsequent alkylation wtih 1,1- difluoro-2-iodo-ethane and acidic Boc deprotection |
| 50 | 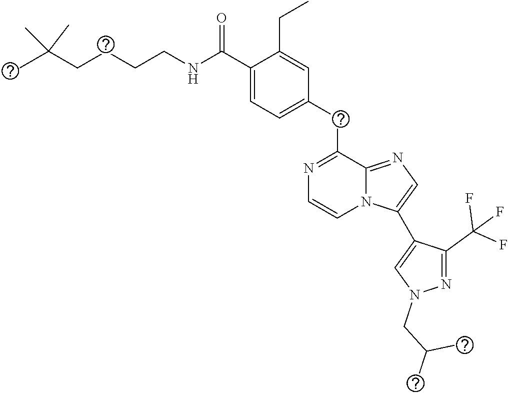 [SMILES: CCc1cc(Cc2nccn3c(-c4cn(CC(C)C)nc4C(F)(F)F)cnc23)ccc1C(=O)NCCCCC(C)(C)C] | N-[2-(2-amino-2- methylpropoxy) ethyl]-4-[[3-[1-(2,2- difluoroethyl)-3- (trifluoromethyl) pyrazol-4- yl]imidazo[1,2- a]pyrazin-8- yl]amino]-2- ethylbenzamide | 594.6 | 595.4 | in analogy to the procedures for example 1: amide coupling of 2- ethyl-4-[(3-iodoimidazo[1,2- a]pyrazin-8-yl)amino]benzoic acid, hydrochloride with tert-butyl (1- (2-aminoethoxy)-2-methylpropan- 2-yl)carbamate, subsequent Suzuki reaction with (3-(trifluoromethyl)- 1H-pyrazol-4-yl)boronic acid, alkylation with 1,1-difluoro-2- iodo-ethane and acidic Boc deprotection |
| 51 | 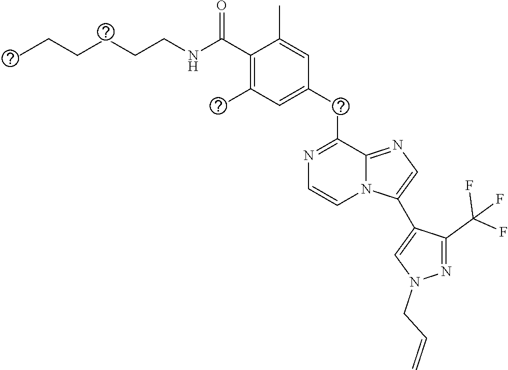 [SMILES: C=CCn1cc(-c2cnc3c(Cc4cc(C)c(C(=O)NCCCCCC)c(C)c4)nccn23)c(C(F)(F)F)n1] | N-[2-(2- aminoethoxy)ethyl]- 2-fluoro-6- methyl-4-[[3-[1- prop-2-enyl-3- (trifluoromethyl) pyrazol-4- yl]imidazo[1,2- a]pyrazin-8- yl]amino] benzamide | 546.5 | 547.3 | in analogy to the procedures for example 4: amide coupling of 2- fluoro-6-methyl-4-[[3-[3- (trifluoromethyl)-1H-pyrzol-4- yl]imidazo[1,2-a]pyrazin-8- yl]amino]benzoic acid with tert- butyl (2-92- aminoethoxy)ethyl)carbamate, subsequent alkylation with 3- bromoprop-1-ene and acidic Boc deprotection |
| 52 | 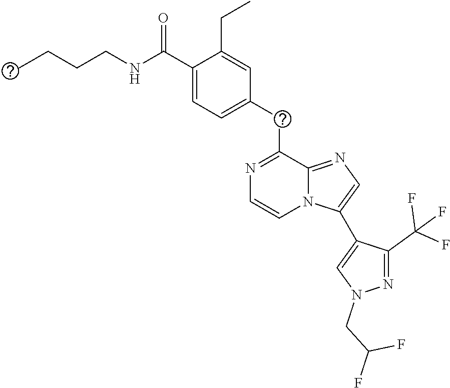 [SMILES: CCCCNC(=O)c1ccc(Cc2nccn3c(-c4cn(CC(F)F)nc4C(F)(F)F)cnc23)cc1CC] | N-(3- aminopropyl)-4- [[3-[1-(2,2- difluoroethyl)-3- (trifluoromethyl) pyrazol-4- yl]imidazo[1,2- a]pyrazin-8- yl]amino]-2- ethylbenzamide | 536.5 | 537.3 | in analogy to the procedures for example 1: amide coupling of 2- ethyl-4-[(3-iodoimidazo[1,2- a]pyrazin-8-yl)amino]benzoic acid, hydrochloride with tert-butyl (3- aminopropyl)carbamate, subsequent Suzuki reaction with (3-(trifluoromethyl)-1H-pyrazol-4- yl)boronic acid, alkylation with 1,1-difluoro-2-iodo-ethane and acidic Boc deprotection |
| 53 | 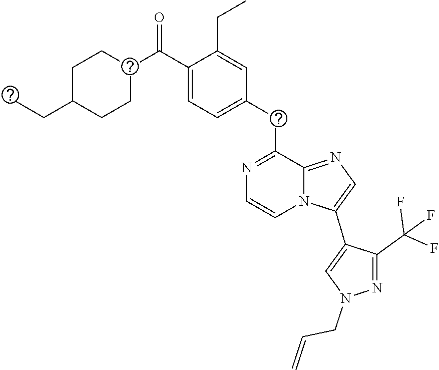 [SMILES: C=CCn1cc(-c2cnc3c(Cc4ccc(C(=O)C5CCC(CC)CC5)c(CC)c4)nccn23)c(C(F)(F)F)n1] | [4- (aminomethyl) piperidin-1-yl]-[2- ethyl-4-[[3-[1- prop-2-enyl-3- (trifluoromethyl) pyrazol-4- yl]imidazo[1,2- a]pyrazin-8- yl]amino]phenyl] methanone | 552.6 | 553.4 | in analogy to the procedures for example 1: amide coupling of 2- ethyl-4-[(3-iodoimidazo[1,2- a]pyrazin-8-yl)amino]benzoic acid, hydrochloride with tert-butyl (piperidin-4-ylmethyl)carbamate, subsequent Suzuki reaction with (3-(trifluoromethyl)-1H-pyrazol-4- yl)boronic acid, alkylation with 3- bromoprop-1-ene and acidic Boc deprotection |
| 54 | 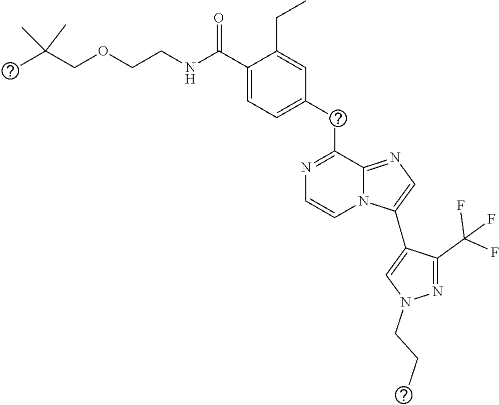 [SMILES: CCCn1cc(-c2cnc3c(Cc4ccc(C(=O)NCCOCC(C)(C)C)c(CC)c4)nccn23)c(C(F)(F)F)n1] | N-[2-(2-amino-2- methylpropoxy) ethyl]-2-ethyl-4-[[3- [1-(2-fluoroethyl)- 3- (trifluoromethyl) pyrazol-4- yl]imidazo[1,2- a]pyrazin-8- yl]amino]benzamide | 576.6 | 577.4 | in analogy to the procedures for example 1: amide coupling of 2- ethyl-4-[(3-iodoimidazo[1,2- a]pyrazin-8-yl)amino]benzoic acid, hydrochloride with tert-butyl (1- (2-aminoethoxy)-2-methylpropan- 2-yl)carbamate, subsequent Suzuki reaction with (3-(trifluoromethyl)- 1H-pyrazol-4-yl)boronic acid, alkylation with 1-bromo-2- fluoroethane and acidic Boc deprotection |
| 55 | 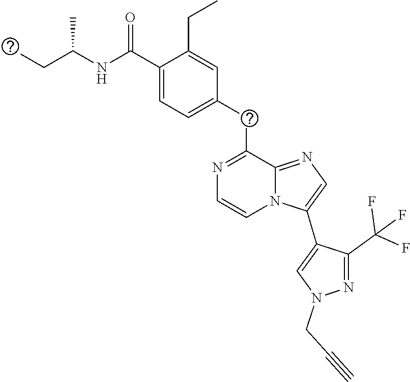 [SMILES: C#CCn1cc(-c2cnc3c(Cc4ccc(C(=O)N[C@@H](C)CC)c(CC)c4)nccn23)c(C(F)(F)F)n1] | N-[(2S)-1- aminopropan-2- yl]-2-ethyl-4-[[3- [1-prop-2-ynyl-3- (trifluoromethyl) pyrazol-4- yl]imidazo[1,2- a]pyrazin-8- yl]amino]benzamide | 510.5 | 511.3 | in analogy to the procedures for example 1: amide coupling of 2- ethyl-4-[(3-iodoimidazo[1,2- a]pyrazin-8-yl)amino]benzoic acid, hydrochloride with tert-butyl (S)- (2-aminopropyl)carbamate, subsequent Suzuki reaction with (3-(trifluoromethyl)-1H-pyrazol-4- yl)boronic acid, alkylation with 3- bromoprop-1-yne and acidic Boc deprotection |
| 56 | 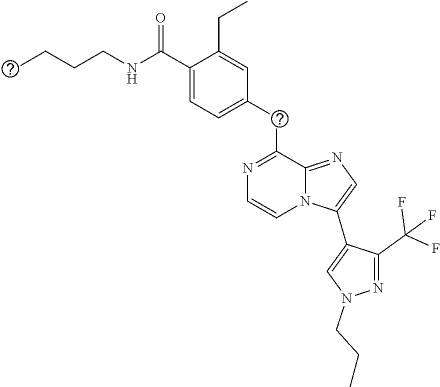 [SMILES: CCCCNC(=O)c1ccc(Cc2nccn3c(-c4cn(CCC)nc4C(F)(F)F)cnc23)cc1CC] | N-(3- aminopropyl)-2- ethyl-4-[[3-[1-(2- fluoroethyl)-3- (trifluoromethyl) pyrazol-4- yl]imidazo[1,2- a]pyrazin-8- yl]amino]benzamide | 518.5 | 519.4 | in analogy to the procedures for example 1: amide coupling of 2- ethyl-4-[(3-iodoimidazo[1,2- a]pyrazin-8-yl)amino]benzoic acid, hydrochloride with tert-butyl (3- aminopropyl)carbamate, subsequent Suzuki reaction with (3-(trifluoromethyl)-1H-pyrazol-4- yl)boronic acid, alkylation with 1- bromo-2-fluoroethane and acidic Boc deprotection |
| 57 | 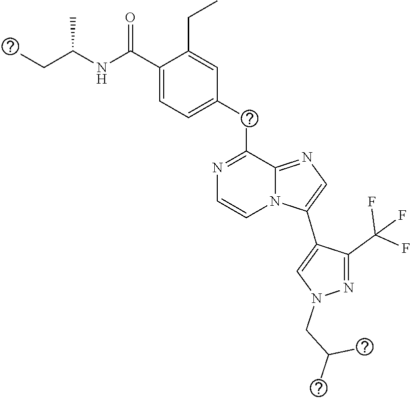 [SMILES: CCc1cc(Cc2nccn3c(-c4cn(CC(C)C)nc4C(F)(F)F)cnc23)ccc1C(=O)N[C@@H](C)CC] | N-[(2S)-1- aminopropan-2- yl]-4-[[3-[1-(2,2- difluoroethyl)-3- (trifluoromethyl) pyrazol-4- yl]imidazo[1,2- a]pyrazin-8- yl]amino]-2- ethylbenzamide | 536.5 | 537.3 | in analogy to the procedures for example 1: amide coupling of 2- ethyl-4-[(3-iodoimidazo[1,2- a]pyrazin-8-yl)amino]benzoic acid, hydrochloride with tert-butyl (S)- (2-aminopropyl)carbamate, subsequent Suzuki reaction with (3-(trifluoromethyl)-1H-pyrazol-4- yl)boronic acid, alkylation with 1,1-difluoro-2-iodo-ethane and acidic Boc deprotection |
| 58 | 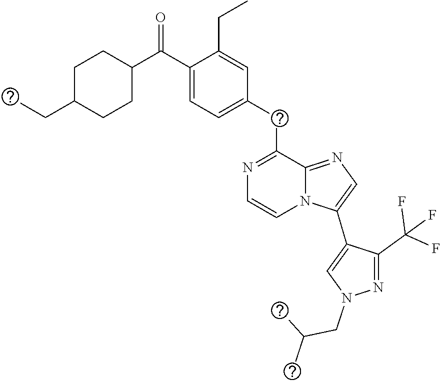 [SMILES: CCc1cc(Cc2nccn3c(-c4cn(CC(C)C)nc4C(F)(F)F)cnc23)ccc1C(=O)C1CCC(CC)CC1] | [4- (aminomethyl) piperidin-1-yl]-[4- [[3-[1-(2,2- difluoroethyl)-3- (trifluoromethyl) pyrazol-4- yl]imidazo[1,2- a]pyrazin-8- yl]amino]-2- ethylphenyl] methanone | 576.6 | 577.4 | in analogy to the procedures for example 1: amide coupling of 2- ethyl-4-[(3-iodoimidazo[1,2- a]pyrazin-8-yl)amino]benzoic acid, hydrochloride with tert-butyl (piperidin-4-ylmethyl)carbamate, subsequent Suzuki reaction with (3-(trifluoromethyl)-1H-pyrazol-4- yl)boronic acid, alkylation with 1,1-difluoro-2-iodo-ethane and acidic Boc deprotection |
| 59 | 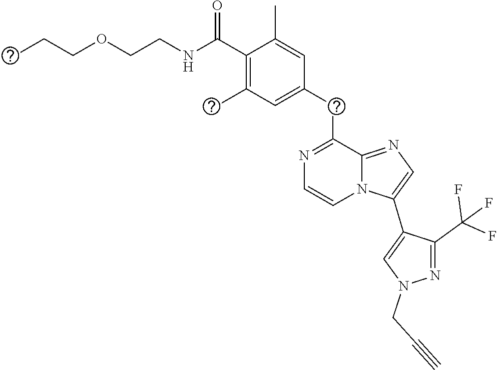 [SMILES: C#CCn1cc(-c2cnc3c(Cc4cc(C)c(C(=O)NCCOCCC)c(C)c4)nccn23)c(C(F)(F)F)n1] | N-[2-(2- aminoethoxy)ethyl]- 2-fluoro-6- methyl-4-[[3-[1- prop-2-ynyl-3- (trifluoromethyl) pyrazol-4- yl]imidazo[1,2- a]pyrazin-8- yl]amino] benzamide | 544.5 | 545.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (2-(2- aminoethoxy)ethyl)carbamate, subsequent alkylation with 3- bromoprop-1-yne and acidic Boc deprotection |
| 60 | 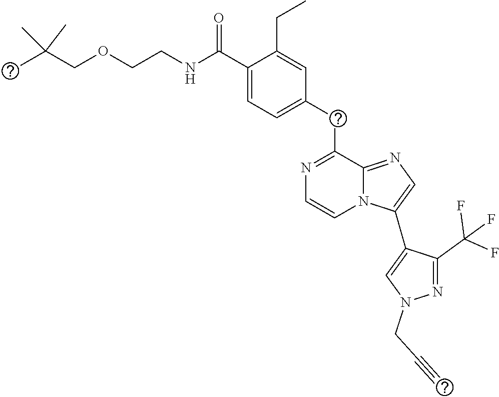 [SMILES: C#CCn1cc(-c2cnc3c(Cc4ccc(C(=O)NCCOCC(C)(C)C)c(CC)c4)nccn23)c(C(F)(F)F)n1] | N-[2-(2-amino-2- methylpropoxy) ethyl]-4-[[3-[1- (cyanomethyl)-3- (trifluoromethyl) pyrazol-4- yl]imidazo[1,2- a]pyrazin-8- yl]amino]-2- ethylbenzamide | 569.6 | 570.4 | in analogy to the procedures for example 1: amide coupling of 2- ethyl-4-[(3-iodoimidazo[1,2- a]pyrazin-8-yl)amino]benzoic acid, hydrochloride with tert-butyl (1- (2-aminoethoxy)-2-methylpropan- 2-yl)carbamate, subsequent Suzuki reaction with (3-(trifluoromethyl)- 1H-pyrazol-4-yl)boronic acid, alkylation with 2-iodoacetonitrile and acidic Boc deprotection |
| 61 | 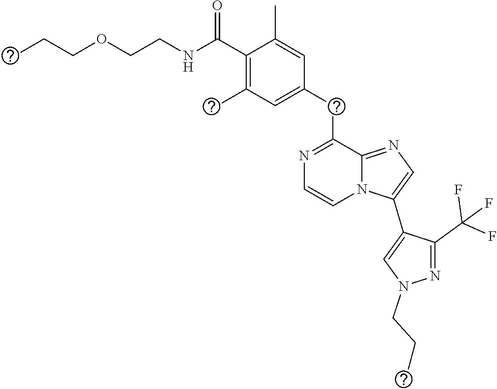 [SMILES: CCCOCCNC(=O)c1c(C)cc(Cc2nccn3c(-c4cn(CCC)nc4C(F)(F)F)cnc23)cc1C] | N-[2-(2- aminoethoxy)ethyl]- 2-fluoro-4-[[3- [1-(2-fluoroethyl)- 3- (trifluoromethyl) pyrazol-4- yl]imidazo[1,2- a]pyrazin-8- yl]amino]-6- methylbenzamide | 552.5 | 553.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (2-(2- aminoethoxy)ethyl)carbamate, subsequent alkylation with 1- bromo-2-fluoroethane and acidic Boc deprotection |
| 62 | 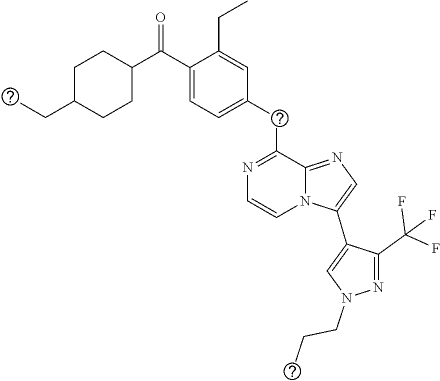 [SMILES: CCCn1cc(-c2cnc3c(Cc4ccc(C(=O)C5CCC(CC)CC5)c(CC)c4)nccn23)c(C(F)(F)F)n1] | [4- (aminomethyl) piperidin-1-yl]-[2- ethyl-4-[[3-[1-(2- fluoroethyl)-3- (trifluoromethyl) pyrazol-4- yl]imidazo[1,2- a]pyrazin-8- yl]amino]phenyl] methanone | 558.6 | 559.3 | in analogy to the procedures for example 1: amide coupling of 2- ethyl-4-[(3-iodoimidazo[1,2- a]pyrazin-8-yl)amino]benzoic acid, hydrochloride with tert-butyl (piperidin-4-ylmethyl)carbamate, subsequent Suzuki reaction with (3-(trifluoromethyl)-1H-pyrazol-4- yl)boronic acid, alkylation with 1- bromo-2-fluoroethane and acidic Boc Boc deprotection |
| 63 | 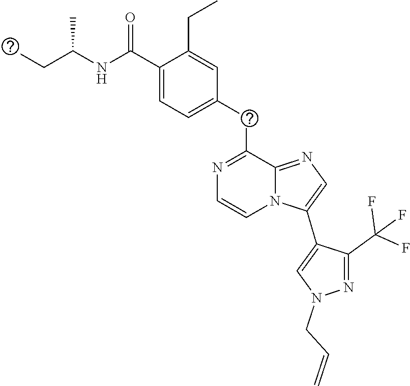 [SMILES: C=CCn1cc(-c2cnc3c(Cc4ccc(C(=O)N[C@@H](C)CC)c(CC)c4)nccn23)c(C(F)(F)F)n1] | N-[(2S)-1- aminopropan-2- yl]-2-ethyl-4-[[3- [1-prop-2-enyl-3- (trifluoromethyl) pyrazol-4- yl]imidazo[1,2- a]pyrazin-8- yl]amino]benzamide | 512.5 | 513.3 | in analogy to the procedures for example 1: amide coupling of 2- ethyl-4-[(3-iodoimidazo[1,2- a]pyrazin-8-yl)amino]benzoic acid, hydrochloride with tert-butyl (S)- (2-aminopropyl)carbamate, subsequent Suzuki reaction with (3-(trifluoromethyl)-1H-pyrazol-4- yl)boronic acid, alkylation with 3- bromoprop-1-ene and acidic Boc deprotection |
| 64 | 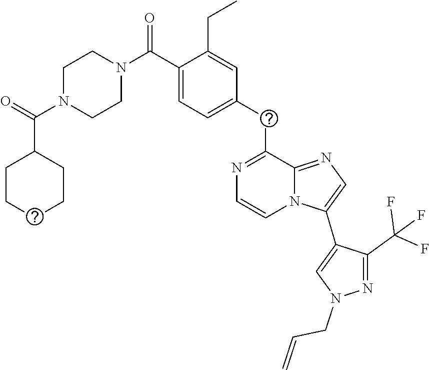 [SMILES: C=CCn1cc(-c2cnc3c(Cc4ccc(C(=O)N5CCN(C(=O)C6CCCCC6)CC5)c(CC)c4)nccn23)c(C(F)(F)F)n1] | [2-ethyl-4-[[3-[1- prop-2-enyl-3- (trifluoromethyl) pyrazol-4- yl]imidazo[1,2- a]pyrazin-8- yl]amino]phenyl]- [4-(piperidine-4- carbonyl)piperazin- 1-yl]methanone | 635.7 | 636.4 | in analogy to the procedures for example 5: alkylation of tert-butyl 4-[4-[2-ethyl-4-[[3-[3- (trifluoromethyl)-1H-pyrazol-4- yl]imidazo[1,2-a]pyrazin-8- yl]amino]benzoyl]piperazine-1- carbonyl]piperidine-1-carboxylate with 3-bromoprop-1-ene and subsequent acidic Boc deprotection |
| 65 | 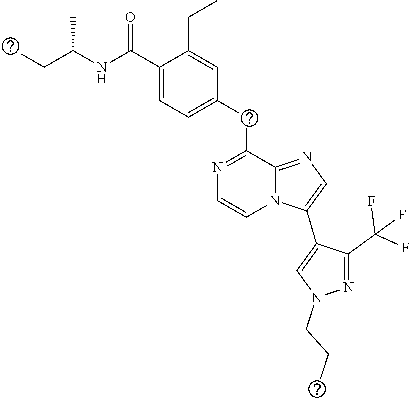 [SMILES: CCCn1cc(-c2cnc3c(Cc4ccc(C(=O)N[C@@H](C)CC)c(CC)c4)nccn23)c(C(F)(F)F)n1] | N-[(2S)-1- aminopropan-2- yl]-2-ethyl-4-[[3- [1-(2-fluoroethyl)- 3- (trifluoromethyl) pyrazol-4- yl]imidazo[1,2- a]pyrazin-8- yl]amino]benzamide | 518.5 | 519.3 | in analogy to the procedures for example 1: amide coupling of 2- ethyl-4-[(3-iodoimidazo[1,2- a]pyrazin-8-yl)amino]benzoic acid, hydrochloride with tert-butyl (S)- (2-aminopropyl)carbamate, subsequent Suzuki reaction with (3-(trifluoromethyl)-1H-pyrazol-4- yl)boronic acid, alkylation with 1- bromo-2-fluoroethane and acidic Boc deprotection |
| 66 | 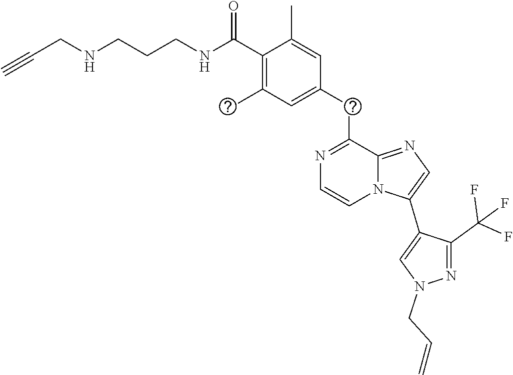 [SMILES: C#CCNCCCNC(=O)c1c(C)cc(Cc2nccn3c(-c4cn(CC=C)nc4C(F)(F)F)cnc23)cc1C] | 2-fluoro-6- methyl-4-[[3-[1- prop-2-enyl-3- (trifluoromethyl) pyrazol-4- yl]imidazo[1,2- a]pyrazin-8- yl]amino]-N-[3- (prop-2- ynylamino)propyl] benzamide | 554.5 | 555.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (3-aminopropyl)(prop-2-yn- 1-yl)carbamate, subsequent alkylation with 3-bromoprop-1-ene and acidic Boc deprotection |
| 67 | 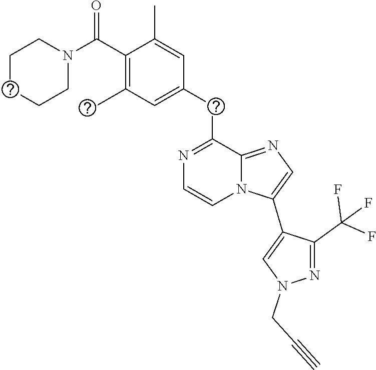 [SMILES: C#CCn1cc(-c2cnc3c(Cc4cc(C)c(C(=O)N5CCCCC5)c(C)c4)nccn23)c(C(F)(F)F)n1] | [2-fluoro-6- methyl-4-[[3-[1- prop-2-ynyl-3- (trifluoromethyl) pyrazol-4- yl]imidazo[1,2- a]pyrazin-8- yl]amino]phenyl]- piperazin-1- ylmethanone | 526.5 | 527.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with 1-(tert- butoxycarbonyl)piperidine-4- carboxylic acid, subsequent alkylation with 3-bromoprop-1- yne and acidic Boc deprotection |
| 68 | 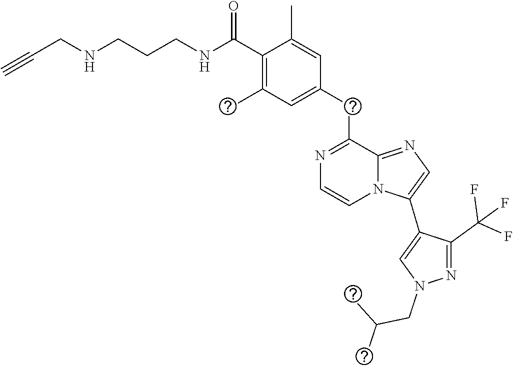 [SMILES: C#CCNCCCNC(=O)c1c(C)cc(Cc2nccn3c(-c4cn(CC(C)C)nc4C(F)(F)F)cnc23)cc1C] | 4-[[3-[1-(2,2- difluoroethyl)-3- (trifluoromethyl) pyrazol-4- yl]imidazo[1,2- a]pyrazin-8- yl]amino]-2- fluoro-6-methyl- N-[3-(prop-2- ynylamino)propyl] benzamide | 578.5 | 579.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (3-aminopropyl)(prop-2-yn- 1-yl)carbamate, subsequent alkylation with 1,1-difluoro-2- iodo-ethane and acidic Boc deprotection |
| 69 | 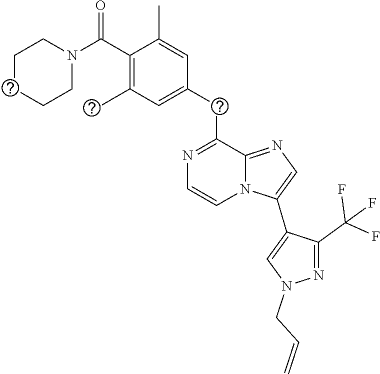 [SMILES: C=CCn1cc(-c2cnc3c(Cc4cc(C)c(C(=O)N5CCCCC5)c(C)c4)nccn23)c(C(F)(F)F)n1] | [2-fluoro-6- methyl-4-[[3-[1- prop-2-enyl-3- (trifluoromethyl) pyrazol-4- yl]imidazo[1,2- a]pyrazin-8- yl]amino]phenyl]- piperazin-1- ylmethanone | 528.5 | 529.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butoxycarbonyl)piperidine-4- carboxylic acid, subsequent alkylation with 3-bromoprop-1-ene and acidic Boc deprotection |
| 70 | 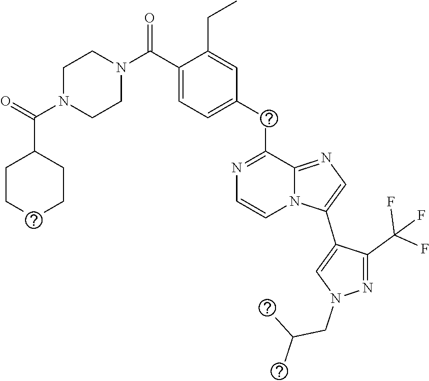 [SMILES: CCc1cc(Cc2nccn3c(-c4cn(CC(C)C)nc4C(F)(F)F)cnc23)ccc1C(=O)N1CCN(C(=O)C2CCCCC2)CC1] | [4-[[3-[1-(2,2- difluoroethyl)-3- (trifluoromethyl) pyrazol-4- yl]imidazo[1,2- a]pyrazin-8- yl]amino]-2- ethylphenyl]-[4- (piperidine-4- carbonyl)piperazin- 1-yl]methanone | 659.7 | 660.4 | in analogy to the procedures for example 5: alkylation of tert-butyl 4-[4-[2-ethyl-4-[[3-[3- (trifluoromethyl)-1H-pyrazol-4- yl]imidazo[1,2-a]pyrazin-8- yl]amino]benzoyl]piperazine-1- carbonyl]piperidine-1-carboxylate with 1,1-difluoro-2-iodo-ethane and subsequent acidic Boc deprotection |
| 71 | 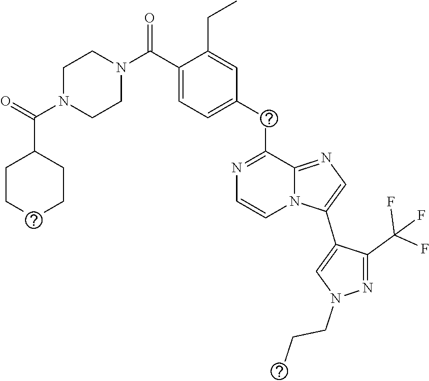 [SMILES: CCCn1cc(-c2cnc3c(Cc4ccc(C(=O)N5CCN(C(=O)C6CCCCC6)CC5)c(CC)c4)nccn23)c(C(F)(F)F)n1] | [2-ethyl-4-[[3-[1- (2-fluoroethyl)-3- (trifluoromethyl) pyrazol-4- yl]imidazo[1,2- a]pyrazin-8- yl]amino]phenyl]- [4-(piperidine-4- carbonyl)piperazin- 1-yl]methanone | 641.7 | 642.5 | in analogy to the procedures for example 5: alkylation of tert-butyl 4-[4-[2-ethyl-4-[[3-[3- (trifluoromethyl)-1H-pyrazol-4- yl]imidazo[1,2-a]pyrazin-8- yl]amino]benzoyl]piperazine-1- carbonyl]piperidine-1-carboxylate with 1-bromo-2-fluoroethane and subsequent acidic Boc deprotection |
| 72 | 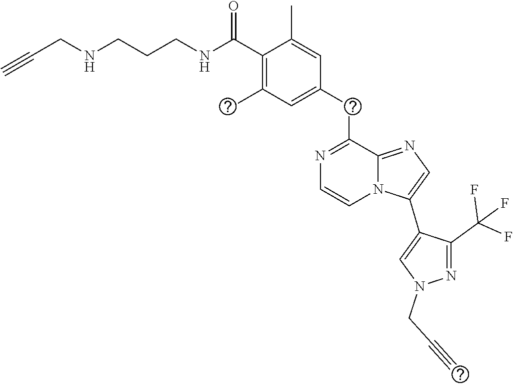 [SMILES: C#CCNCCCNC(=O)c1c(C)cc(Cc2nccn3c(-c4cn(CC#C)nc4C(F)(F)F)cnc23)cc1C] | 2-fluoro-6- methyl-N-[3- (prop-2- ynylamino)propyl]- 4-[[3-[1-prop-2- ynyl-3- (trifluoromethyl) pyrazol-4- yl]imidazo[1,2- a]pyrazin-8- yl]amino]benzamide | 552.5 | 553.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (3-aminopropyl)(prop-2-yn- 1-yl)carbamate, subsequent alkylation with 3-bromoprop-1- yne and acidic Boc deprotection |
| 73 | 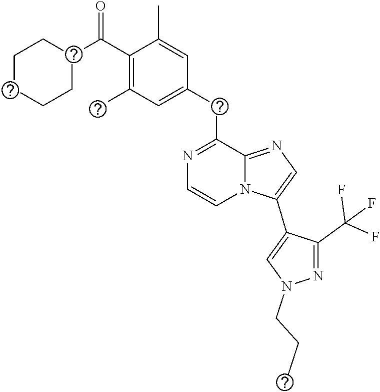 [SMILES: CCCn1cc(-c2cnc3c(Cc4cc(C)c(C(=O)C5CCCCC5)c(C)c4)nccn23)c(C(F)(F)F)n1] | [2-fluoro-4-[[3- [1-(2-fluoroethyl)- 3- (trifluoromethyl) pyrazol-4- yl]imidazo[1,2- a]pyrazin-8- yl]amino]-6- methylphenyl]- piperazin-1- ylmethanone | 534.5 | 535.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with 1-(tert- butoxycarbonyl)piperidine-4- carboxylic acid, subsequent alkylation with 1-bromo-2- fluoroethane and acidic Boc deprotection |
| 74 | 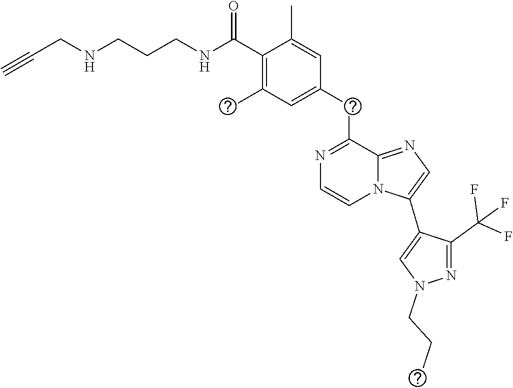 [SMILES: C#CCNCCCNC(=O)c1c(C)cc(Cc2nccn3c(-c4cn(CCC)nc4C(F)(F)F)cnc23)cc1C] | 2-fluoro-4-[[3-[1- (2-fluoroethyl)-3- (trifluoromethyl) pyrazol-4- yl]imidazo[1,2- a]pyrazin-8- yl]amino]-6- methyl-N-[3- (prop-2- ynylamino)propyl] benzamide | 560.5 | 561.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (3-aminopropyl)(prop-2-yn- 1-yl)carbamate, subsequent alkylation with 1-bromo-2- fluoroethane and acidic Boc deprotection |
| 75 | 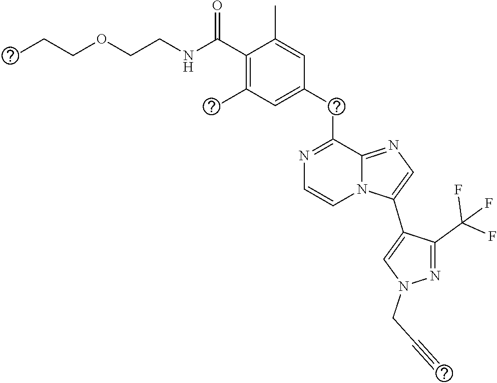 [SMILES: C#CCn1cc(-c2cnc3c(Cc4cc(C)c(C(=O)NCCOCCC)c(C)c4)nccn23)c(C(F)(F)F)n1] | N-[2-(2- aminoethoxy)ethyl]- 4-[[3-[1- (cyanomethyl)-3- (trifluoromethyl) pyrazol-4- yl]imidazo[1,2- a]pyrazin-8- yl]amino]-2- fluoro-6- methylbenzamide | 545.5 | 546.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (2-(2- aminoethoxy)ethyl)carbamate, subsequent alkylation with 2- iodoacetonitrile and acidic Boc deprotection |
| 76 | 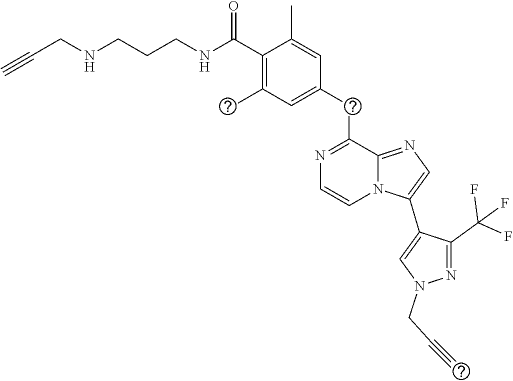 [SMILES: C#CCNCCCNC(=O)c1c(C)cc(Cc2nccn3c(-c4cn(CC#C)nc4C(F)(F)F)cnc23)cc1C] | 4-[[3-[1- (cyanomethyl)-3- (trifluoromethyl) pyrazol-4- yl]imidazo[1,2- a]pyrazin-8- yl]amino]-2- fluoro-6-methyl- N-[3-(prop-2- ynylamino)propyl] benzamide | 553.5 | 554.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (3-aminopropyl)(prop-2-yn- 1-yl)carbamate, subsequent alkylation with 2-iodoacetonitrile and acidic Boc deprotection |
| 77 | 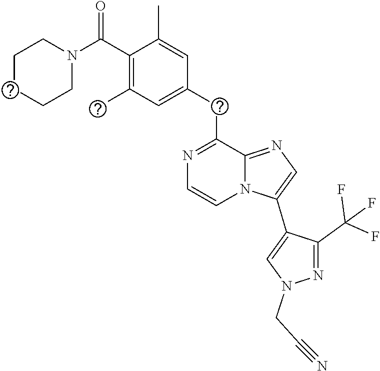 [SMILES: Cc1cc(Cc2nccn3c(-c4cn(CC#N)nc4C(F)(F)F)cnc23)cc(C)c1C(=O)N1CCCCC1] | 2-[4-[8-[3-fluoro- 5-methyl-4- (piperazine-1- carbonyl)anilino] imidazo[1,2- a]pyrazin-3-yl]-3- (trifluoromethyl) pyrazol-1- yl]acetonitrile | 527.5 | 528.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with 1-(tert- butoxycarbonyl)piperidine-4- carboxylic acid, subsequent alkylation with 2-iodoacetonitrile and acidic Boc deprotection |
| 78 | 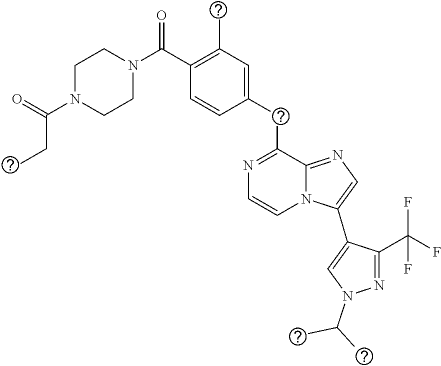 [SMILES: CCC(=O)N1CCN(C(=O)c2ccc(Cc3nccn4c(-c5cn(C(C)C)nc5C(F)(F)F)cnc34)cc2C)CC1] | 2-amino-1-[4-[2- chloro-4-[[3-[1- (difluoromethyl)- 3- (trifluoromethyl) pyrazol-4- yl]imidazo[1,2- a]pyrazin-8- yl]amino]benzoyl] piperazin-1- yl]ethanone | 597.9 | 598.3 | in analogy to the procedures for example 6: alkylation of tert-butyl N-[2-[4-[2-chloro-4-[[3-[3- (trifluoromethyl)-1H-pyrazol-4- yl]imidazo[1,2-a]pyrazin-8- yl]amino]benzoyl]piperazin-1-yl]- 2-oxo-ethyl]carbamate with methyl 2-chloro-2,2- difluoroacetate and acidic Boc deprotection |
| 79 | 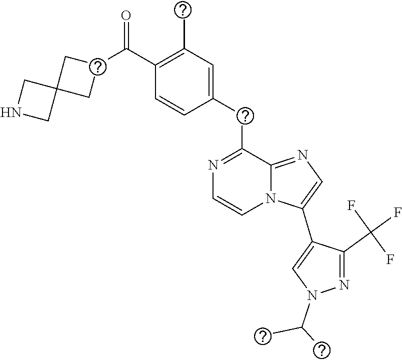 [SMILES: Cc1cc(Cc2nccn3c(-c4cn(C(C)C)nc4C(F)(F)F)cnc23)ccc1C(=O)C1CC2(CNC2)C1] | [2-chloro-4-[[3- [1- (difluoromethyl)- 3- (trifluoromethyl) pyrazol-4- yl]imidazo[1,2- a]pyrazin-8- yl]amino]phenyl]- (2,6- diazaspiro[3.3] heptan-2- yl)methanone | 552.9 | 551.3 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl 2,6-diazaspiro[3.3]heptane- 2-carboxylate hemioxalate [CAS#1041026-71-4], subsequent alkylation with methyl 2-chloro- 2,2-difluoroacetate and acidic Boc deprotection |
| 80 | 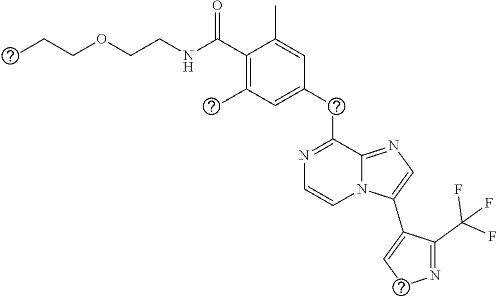 [SMILES: CCCOCCNC(=O)c1c(C)cc(Cc2nccn3c(C4=CCN=C4C(F)(F)F)cnc23)cc1C] | N-[2-(2- aminoethoxy)ethyl]- 2-fluoro-6- methyl-4-[[3-[3- (trifluoromethyl)- 1H-pyrazol-4- yl]imidazo[1,2- a]pyrazin-8- yl]amino]benzamide | 506.5 | 507.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (2-(2- aminoethoxy)ethyl)carbamate and subsequent acidic Boc deprotection |
| 81 | 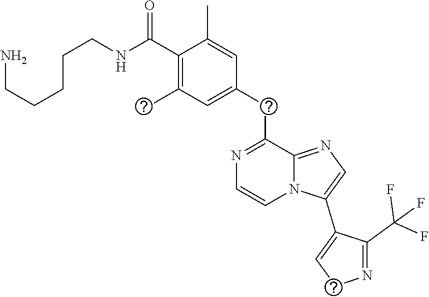 [SMILES: Cc1cc(Cc2nccn3c(C4=CCN=C4C(F)(F)F)cnc23)cc(C)c1C(=O)NCCCCCN] | N-(5- aminopentyl)-2- fluoro-6-methyl- 4-[[3-[3- (trifluoromethyl)- 1H-pyrazol-4- yl]imidazo[1,2- a]pyrazin-8- yl]amino]benzamide | 504.5 | 505.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (5-aminopentyl)carbamate and subsequent acidic Boc deprotection |
| 82 | 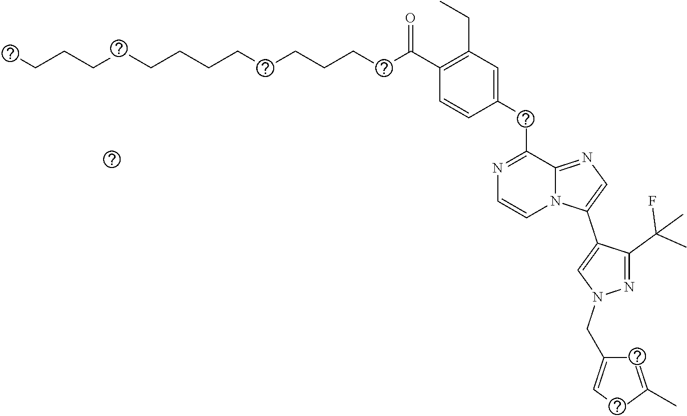 [SMILES: CCCCCCCCCCCCCCC(=O)c1ccc(Cc2nccn3c(-c4cn(CC5=CCC(C)=C5)nc4C(C)(C)F)cnc23)cc1CC] | N-[3-[4-(3- aminopropylamino) butylamino]propyl]- 2-ethyl-4-[[3- [1-[(2-methyl-1,3- thiazol-4- yl)methyl]-3- (trifluoromethyl) pyrazol-4- yl]imidazo[1,2- a]pyrazin-8 yl]amino]benzamide hydrochloride | 711.9 | 712.5 | In analogy to the procedure described for example 2, step 2 the title compound was prepared from tert-butyl N-[3-(tert- butoxycarbonylamino)propyl]-N- [4-[tert-butoxycarbonyl-[3-[[2- ethyl-4-[[3-[3-(trifluoromethyl)- 1H-pyrazol-4-yl]imidazo[1,2- a]pyrazin-8- yl]amino]benzoyl]amino]propyl] amino]butyl]carbamate and 4- (chloromethyl)-2-methylthiazole hydrochloride and subsequent acidic Boc deprotection |
| 83 | 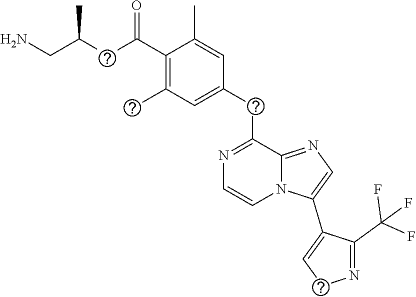 [SMILES: Cc1cc(Cc2nccn3c(C4=CCN=C4C(F)(F)F)cnc23)cc(C)c1C(=O)C[C@H](C)CN] | N-[(2R)-1- aminopropan-2- yl]-2-fluoro-6- methyl-4-[[3-[3- (trifluoromethyl)- 1H-pyrazol-4- yl]imidazo[1,2- a]pyrazin-8- yl]amino]benzamide | 476.4 | 477.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (R)-(2- aminopropyl)carbamate and subsequent acidic Boc deprotection |
| 84 | 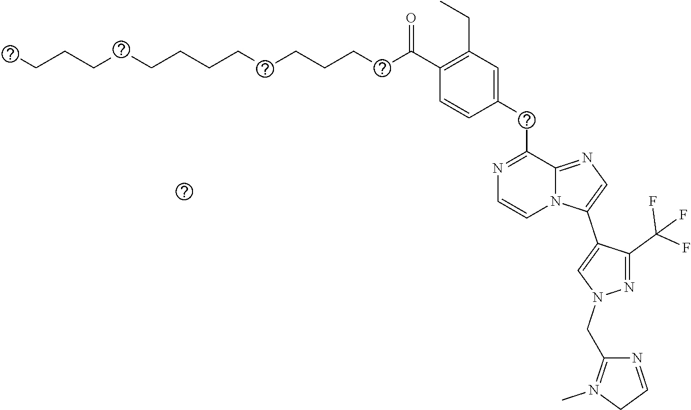 | N-[3-[4-(3- aminopropylamino) butylamino]propyl]- 2-ethyl-4-[[3- [1-[(1- methylimidazol-2- yl)methyl]-3- (trifluoromethyl) pyrazol-4- yl]imidazo[1,2- a]pyrazin-8- yl]amino]benzamide hydrochloride | 694.8 | 695.6 | In analogy to the procedure described for example 2, step 2 the title compound was prepared from tert-butyl N-[3-(tert- butoxycarbonylamino)propyl]-N- [4-[tert-butoxycarbonyl-[3-[[2- ethyl-4-[[3-[3-(trifluoromethyl)- 1H-pyrazol-4-yl]imidazo[1,2- a]pyrazin-8- yl]amino]benzoyl]amino]propyl] amino]butyl]carbamate and 2- (chloromethyl)-1-methyl-1H- imidazole hydrochloride and subsequent acidic Boc deprotection |
| 85 | 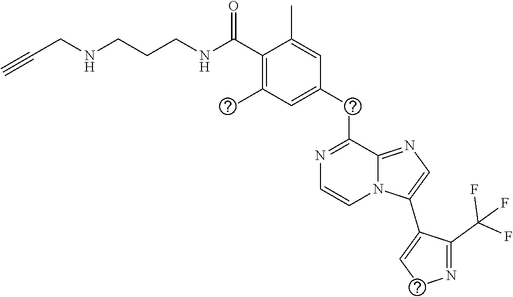 [SMILES: C#CCNCCCNC(=O)c1c(C)cc(Cc2nccn3c(C4=CCN=C4C(F)(F)F)cnc23)cc1C] | 2-fluoro-6- methyl-N-[3- (prop-2- ynylamino)propyl]- 4-[[3-[3- (trifluoromethyl)- 1H-pyrazol-4- yl]imidazo[1,2- a]pyrazin-8- yl]amino]benzamide | 514.5 | 515.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl (3-aminopropyl)(prop-2-yn- 1-yl)carbamate and subsequent acidic Boc deprotection |
| 86 | 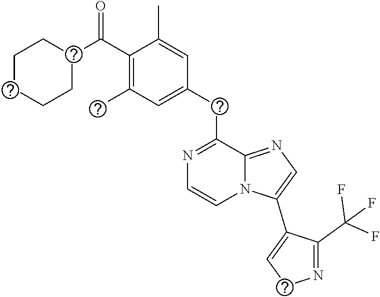 [SMILES: Cc1cc(Cc2nccn3c(C4=CCN=C4C(F)(F)F)cnc23)cc(C)c1C(=O)C1CCCCC1] | [2-fluoro-6- methyl-4-[[3-[3- (trifluoromethyl)- 1H-pyrazol-4- yl]imidazo[1,2- a]pyrazin-8- yl]amino]phenyl]- piperazin-1- ylmethanone | 488.4 | 489.3 | in analogy to the procedures for example 4: amide coupling of 2- fluoro-6-methyl-4-[[3-[3- (trifluoromethyl)-1H-pyrazol-4- yl]imidazo[1,2-a]pyrazin-8- yl]amino]benzoic acid with tert- butyl piperazine-1-carboxylate and subsequent acidic Boc deprotection |
| 87 | 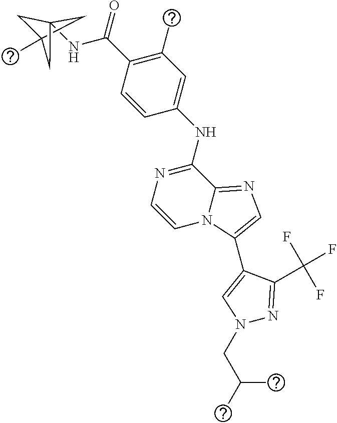 [SMILES: Cc1cc(Nc2nccn3c(-c4cn(CC(C)C)nc4C(F)(F)F)cnc23)ccc1C(=O)NC12CC(C)(C1)C2] | N-(3-amino-1- bicyclo[1.1.1] pentanyl)-2-chloro-4- [[3-[1-(2,2- difluoroethyl)-3- (trifluoromethyl) pyrazol-4- yl]imidazo[1,2- a]pyrazin-8- yl]amino]benzamide | 566.9 | 565.4 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl (3- aminobicyclo[1.1.1]pentan-1- yl)carbamate[CAS#1638767-25- 5] and subsequent alkylation with 1,1-difluoro-2-iodoethane [CAS#598-39-0] and acidic Boc deprotection |
| 88 | 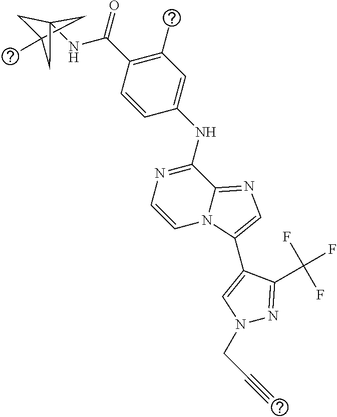 [SMILES: C#CCn1cc(-c2cnc3c(Nc4ccc(C(=O)NC56CC(C)(C5)C6)c(C)c4)nccn23)c(C(F)(F)F)n1] | N-(3-amino-1- bicyclo[1.1.1] pentanyl)-2-chloro-4- [[3-[1- (cyanomethyl)-3- (trifluoromethyl) pyrazol-4- yl]imidazo[1,2- a]pyrazin-8- yl]amino]benzamide | 541.9 | 540.3 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl (3- aminobicyclo[1.1.1]pentan-1- yl)carbamate[CAS#1638767-25- 5] and subsequent alkylation with 2-bromoacetonitrile [CAS#590-17- 0] and acidic Boc deprotection |
| 89 | 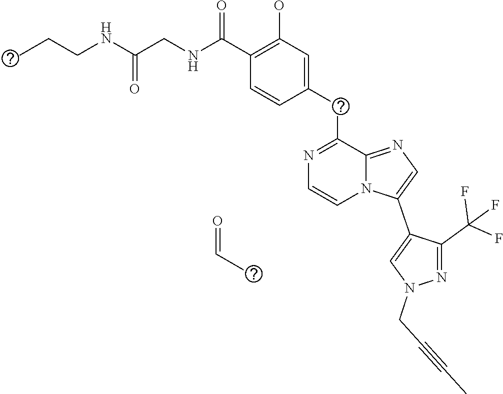 | N-[2-(2- aminoethylamino)- 2-oxoethyl]-4- [[3-[1-but-2-ynyl- 3- (trifluoromethyl) pyrazol-4- yl]imidazo[1,2- a]pyrazin-8- yl]amino]-2- chlorobenzamide formate | 574.0 | 574.1 | in analogy to the procedures for example 8: alkylation of tert-butyl N-[2-[[2-[[2-chloro-4-[[3-[1-(2- fluoroethyl)-3- (trifluoromethyl)pyrazol-4- yl]imidazo[1,2-a]pyrazin-8- yl]amino]benzoyl]amino]acetyl] amino]ethyl]carbamate (example 8, step 4) with 1-bromo-2-butyne and acidic Boc deprotection |
| 90 | 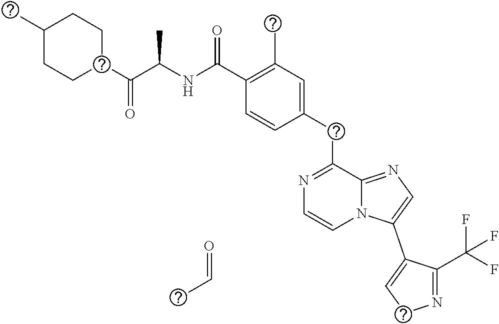 | N-[(2R)-1-(4- aminopiperidin-1- yl)-1-oxopropan- 2-yl]-2-chloro-4- [[3-[3- (trifluoromethyl)- 1H-pyrazol-4- yl]imidazo[1,2- a]pyrazin-8- yl]amino]benzamide formate | 576.0 | 576.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with 4- BOC-aminopiperidine and acidic Boc deprotection |
| 91 | 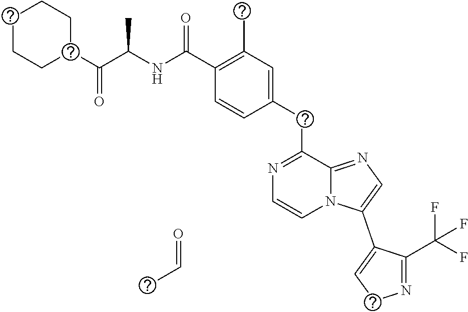 | 2-chloro-N-[(2R)- 1-oxo-1- piperazin-1- ylpropan-2-yl]-4- [[3-[3- (trifluoromethyl)- 1H-pyrazol-4- yl]imidazo[1,2- a]pyrazin-8- yl]amino]benzamide formate | 562.0 | 562.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with 4- BOC-piperazine and acidic Boc deprotection |
| 92 | 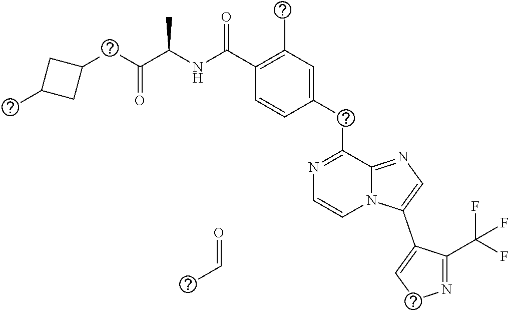 | N-[(2R)-1-[(3- aminocyclobutyl) amino]-1- oxopropan-2-yl]- 2-chloro-4-[[3-[3- (trifluoromethyl)- 1H-pyrazol-4- yl]imidazo[1,2- a]pyrazin-8- yl]amino]benzamide formate | 562.0 | 562.3 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with tert-butyl 3- aminocyclobutylcarbamate and Boc deprotection |
| 93 | 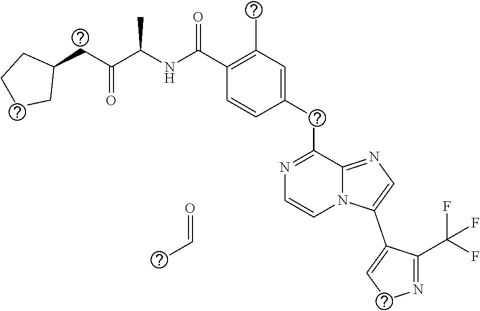 | 2-chloro-N-[(2R)- 1-oxo-1-[[(3R)- pyrrolidin-3- yl]amino]propan- 2-yl]-4-[[3-[3- (trifluoromethyl)- 1H-pyrazol-4- yl]imidazo[1,2- a]pyrazin-8- yl]amino]benzamide formate | 562.0 | 562.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with (R)-(+)-1-BOC-3- aminopyrrolidine and acidic Boc deprotection |
| 94 | 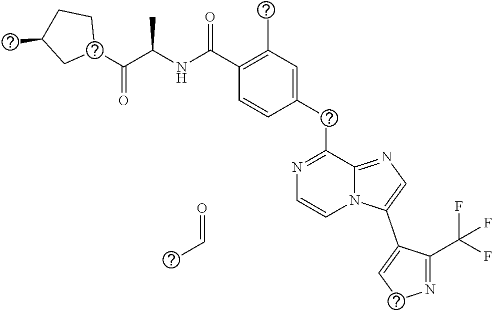 | N-[(2R)-1-[(3S)- 3- aminopyrrolidin- 1-yl]-1- oxopropan-2-yl]- 2-chloro-4-[[3-[3- (trifluoromethyl)- 1H-pyrazol-4- yl]imidazo[1,2- a]pyrazin-8- yl]amino]benzamide formate | 562.0 | 562.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with (S)-3-(BOC-amino)pyrrolidine and acidic Boc deprotection |
| 95 | 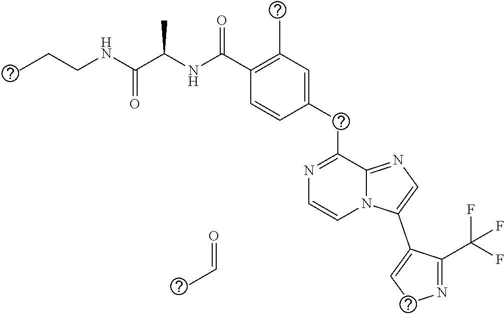 | N-[(2R)-1-(2- aminoethylamino)- 1-oxopropan-2- yl]-2-chloro-4- [[3-[3- (trifluoromethyl)- 1H-pyrazol-4- yl]imidazo[1,2- a]pyrazin-8- yl]amino]benzamide formate | 535.9 | 536.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with N-BOC-ethylenediamine and acidic Boc deprotection |
| 96 | 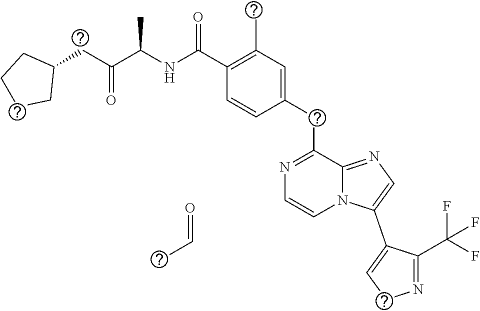 | 2-chloro-N-[(2R)- 1-oxo-1-[[(3S)- pyrrolidin-3- yl]amino]propan- 2-yl]-4-[[3-[3- (trifluoromethyl)- 1H-pyrazol-4- yl]imidazo[1,2- a]pyrazin-8- yl]amino]benzamide formate | 562.0 | 562.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with (S)-3-amino-1-N-BOC-pyrrolidine and acidic Boc deprotection |
| 97 | 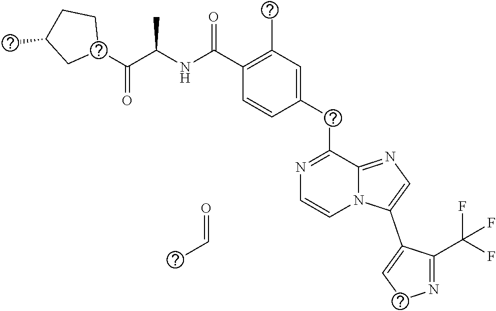 | N-[(2R)-1-[(3R)- 3- aminopyrrolidin- 1-yl]-1- oxopropan-2-yl]- 2-chloro-4-[[3-[3- (trifluoromethyl)- 1H-pyrazol-4- yl]imidazo[1,2- a]pyrazin-8- yl]amino]benzamide formate | 562.0 | 562.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with (R)-3-(BOC-amino)pyrrolidine and acidic Boc deprotection |
| 98 | 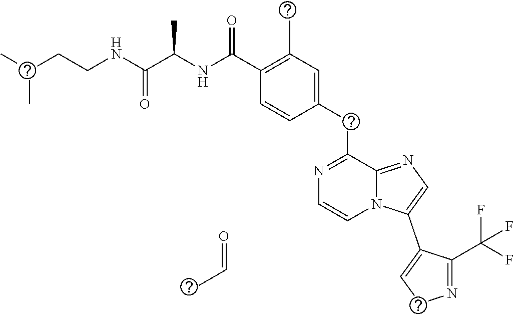 | 2-chloro-N-[(2R)- 1-[2- (dimethylamino) ethylamino]-1- oxopropan-2-yl]- 4-[[3-[3- (trifluoromethyl)- 1H-pyrazol-4- yl]imidazo[1,2- a]pyrazin-8- yl]amino]benzamide formate | 564.0 | 564.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with N,N-dimethylethylenediamine |
| 99 | 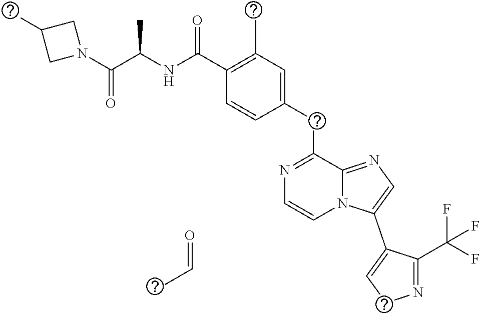 | N-[(2R)-1-(3- aminoazetidin-1- yl)-1-oxopropan- 2-yl]-2-chloro-4- [[3-[3- (trifluoromethyl)- 1H-pyrazol-4- yl]imidazo[1,2- a]pyrazin-8- yl]amino]benzamide formate | 547.9 | 548.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with D-alanine ethyl ester hydrochloride, saponification under basic conditions, amide coupling with 3- N-BOC-amino-azetidine and acidic Boc deprotection |
| 100 | 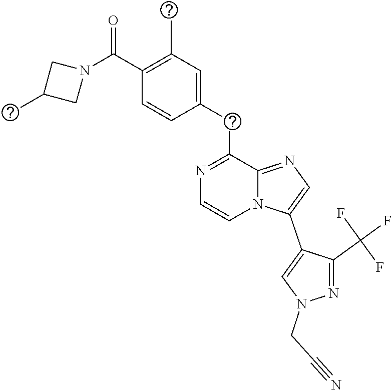 [SMILES: Cc1cc(Cc2nccn3c(-c4cn(CC#N)nc4C(F)(F)F)cnc23)ccc1C(=O)N1CC(C)C1] | 2-[4-[8-[4-(3- aminoazetidine-1- carbonyl)-3- chloroanilino] imidazo[1,2- a]pyrazin-3-yl]-3- (trifluoromethyl) pyrazol-1- yl]acetonitrile | 515.9 | 514.4 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl azetidin-3-ylcarbamate [CAS#91188-13-5] and subsequent alkylation with 2- bromoacetonitrile [CAS#590-17-0] and acidic Boc deprotection |
| 101 | 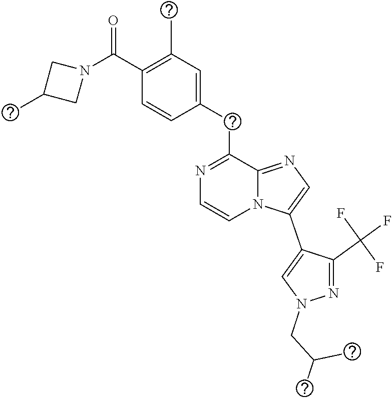 [SMILES: Cc1cc(Cc2nccn3c(-c4cn(CC(C)C)nc4C(F)(F)F)cnc23)ccc1C(=O)N1CC(C)C1] | (3-aminoazetidin- 1-yl)-[2-chloro-4- [[3-[1-(2,2- difluoroethyl)-3- pyrazol-4- yl]imidazo[1,2- a]pyrazin-8- yl]amino]phenyl] methanone | 540.9 | 539.4 | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl azetidin-3-ylcarbamate [CAS#91188-13-5] and subsequent alkylation with 1,1- difluoro-2-iodoethane and acidic Boc deprotection |
| 102 | 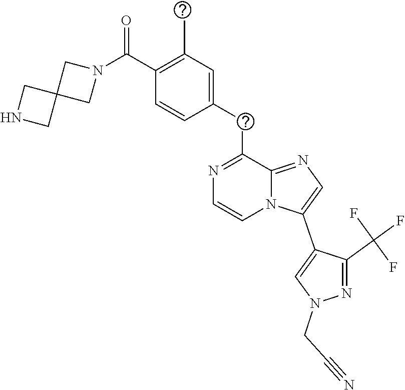 [SMILES: Cc1cc(Cc2nccn3c(-c4cn(CC#N)nc4C(F)(F)F)cnc23)ccc1C(=O)N1CC2(CNC2)C1] | 2-[4-[8-[3- chloro-4-(2,6- diazaspiro[3.3] heptane-2- carbonyl)anilino] imidazo[1,2- a]pyrazin-3-yl]-3- (trifluoromethyl) pyrazol-1- yl]acetonitrile | 541.9 | 540.4 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl 2,6-diazaspiro[3.3]heptane- 2-carboxylate hemioxalate [CAS#1041026-71-4] and subsequent alkylation with 2- bromoacetonitrile [CAS#590-17-0] and acidic Boc deprotection |
| 103 | 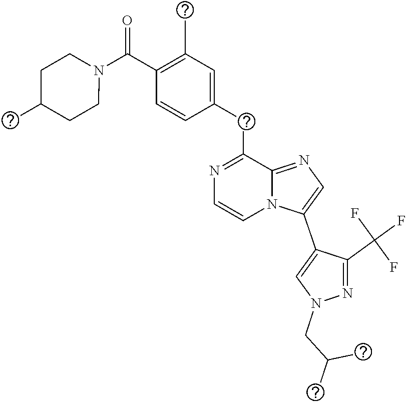 [SMILES: Cc1cc(Cc2nccn3c(-c4cn(CC(C)C)nc4C(F)(F)F)cnc23)ccc1C(=O)N1CCC(C)CC1] | (4- aminopiperidin-1- yl)-[2-chloro-4- [[3-[1-(2,2- difluoroethyl)-3- (trifluoromethyl) pyrazol-4- yl]imidazo[1,2- a]pyrazin-8- yl]amino]phenyl] methanone | 568.9 | 567.4 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl piperidin-4-ylcarbamate [CAS#87120-72-7] and subsequent alkylation with 1,1- difluoro-2-iodoethane [CAS#598- 39-0] and acidic Boc deprotection |
| 104 | 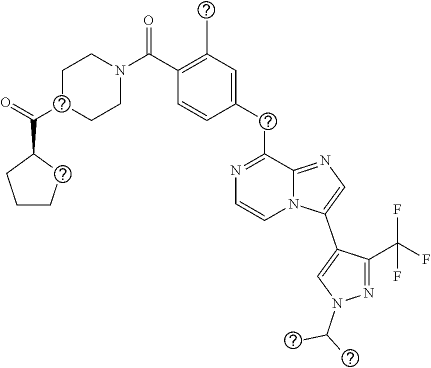 | [2-chloro-4-[[3- [1- (difluoromethyl)- 3- (trifluoromethyl) pyrazol-4- yl]imidazo[1,2- a]pyrazin-8- yl]amino]phenyl]- [4-[(2S)- pyrrolidine-2- carbonyl]piperazin- 1-yl]methanone | 638.0 | 638.5 | in analogy to the procedures for example 6: amide formation with 2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (tert-butoxycarbonyl)-L-proline CAS [15761-39-4] and subsequent alkylation with methyl 2-chloro- 2,2-difluoroacetate and acidic Boc deprotection |
| 105 | 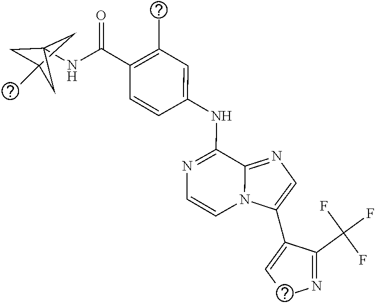 [SMILES: Cc1cc(Nc2nccn3c(C4=CCN=C4C(F)(F)F)cnc23)ccc1C(=O)NC12CC(C)(C1)C2] | N-(3-amino-1- bicyclo[1.1.1] pentanyl)-2-chloro-4- [[3-[3- (trifluoromethyl)- 1H-pyrazol-4- yl]imidazo[1,2- a]pyrazin-8- yl]amino]benzamide | 502.9 | 501.3 [M − H]− | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl (3- aminobicyclo[1.1.1]pentan-1- yl)carbamate [CAS#1638767-25- 5] and subsequent acidic Boc deprotection |
| 106 | 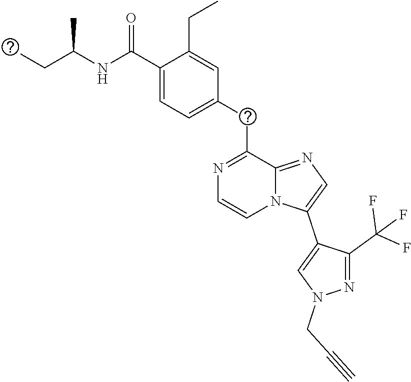 [SMILES: C#CCn1cc(-c2cnc3c(Cc4ccc(C(=O)N[C@H](C)CC)c(CC)c4)nccn23)c(C(F)(F)F)n1] | N-[(2R)-1- aminopropan-2- yl]-2-ethyl-4-[[3- [1-prop-2-ynyl-3- (trifluoromethyl) pyrazol-4- yl]imidazo[1,2- a]pyrazin-8- yl]amino]benzamide | 510.5 | 511.4 | in analogy to the procedures for example 1: amide coupling of 2- ethyl-4-[(3-iodoimidazo[1,2- a]pyrazin-8-yl)amino]benzoic acid, hydrochloride with tert-butyl (R)- (2-aminopropyl)carbamate, subsequent Suzuki reaction with (3-(trifluoromethyl)-1H-pyrazol-4- yl)boronic acid, alkylation with 3- bromoprop-1-yne and acidic Boc deprotection |
| 107 | 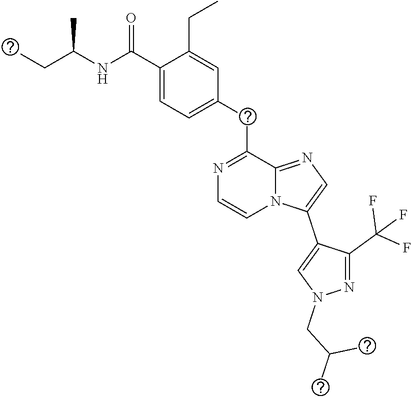 [SMILES: CCc1cc(Cc2nccn3c(-c4cn(CC(C)C)nc4C(F)(F)F)cnc23)ccc1C(=O)N[C@H](C)CC] | N-[(2R)-1- aminopropan-2- yl]-4-[[3-[1-(2,2- difluoroethyl)-3- (trifluoromethyl) pyrazol-4- yl]imidazo[1,2- a]pyrazin-8- yl]amino]-2- ethylbenzamide | 536.5 | 534.4 | in analogy to the procedures for example 1: amide coupling of 2- ethyl-4-[(3-iodoimidazo[1,2- a]pyrazin-8-yl)amino]benzoic acid, hydrochloride with tert-butyl (R)- (2-aminopropyl)carbamate, subsequent Suzuki reaction with (3-(trifluoromethyl)-1H-pyrazol-4- yl)boronic acid, alkylation with 1,1-difluoro-2-iodo-ethane and acidic Boc protection |
| 108 | 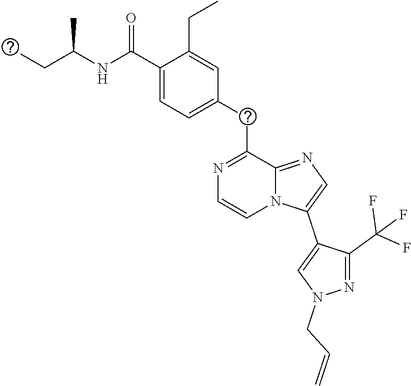 [SMILES: C=CCn1cc(-c2cnc3c(Cc4ccc(C(=O)N[C@H](C)CC)c(CC)c4)nccn23)c(C(F)(F)F)n1] | N-[(2R)-1- aminopropan-2- yl]-2-ethyl-4-[[3- [1-prop-2-enyl-3- (trifluoromethyl) pyrazol-4- yl]imidazo[1,2- a]pyrazin-8- yl]amino]benzamide | 512.5 | 513.4 | in analogy to the procedures for example 1: amide coupling of 2- ethyl-4-[(3-iodoimidazo[1,2- a]pyrazin-8-yl)amino]benzoic acid, hydrochloride with tert-butyl (R)- (2-aminopropyl)carbamate, subsequent Suzuki reaction with (3-(trifluoromethyl)-1H-pyrazol-4- yl)boronic acid, alkylation with 3- bromoprop-1-ene and acidic Boc deprotection |
| 109 | 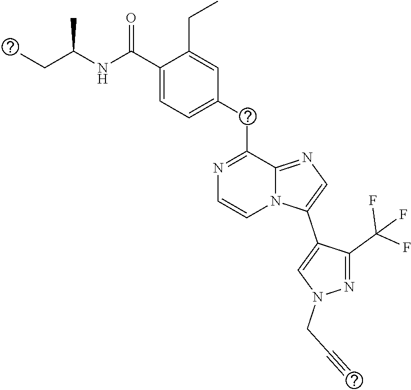 [SMILES: C#CCn1cc(-c2cnc3c(Cc4ccc(C(=O)N[C@H](C)CC)c(CC)c4)nccn23)c(C(F)(F)F)n1] | N-[(2R)-1- aminopropan-2- yl]-4-[[3-[1- (cyanomethyl)-3- (trifluoromethyl) pyrazol-4- yl]imidazo[1,2- a]pyrazin-8- yl]amino]-2- ethylbenzamide | 511.5 | 512.4 | in analogy to the procedures for example 1: amide coupling of 2- ethyl-4-[(3-iodoimidazo[1,2- a]pyrazin-8-yl)amino]benzoic acid, hydrochloride with tert-butyl (R)- (2-aminopropyl)carbamate, subsequent Suzuki reaction with (3-(trifluoromethyl)-1H-pyrazol-4- yl)boronic acid, alkylation with 2- iodoacetonitrile and acidic Boc deprotection |
| 110 | 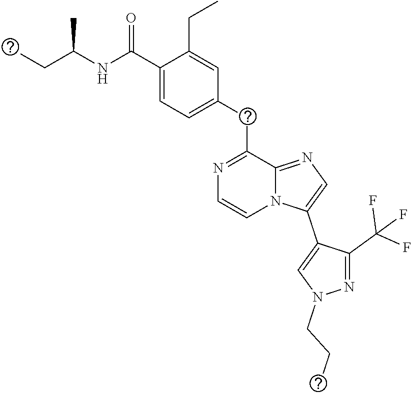 [SMILES: CCCn1cc(-c2cnc3c(Cc4ccc(C(=O)N[C@H](C)CC)c(CC)c4)nccn23)c(C(F)(F)F)n1] | N-[(2R)-1- aminopropan-2- yl]-2-ethyl-4-[[3- [1-(2-fluoroethyl)- 3- (trifluoromethyl) pyrazol-4- yl]imidazo[1,2- a]pyrazin-8- yl]amino]benzamide | 518.5 | 519.4 | in analogy to the procedures for example 1: amide coupling of 2- ethyl-4-[(3-iodoimidazo[1,2- a]pyrazin-8-yl)amino]benzoic acid, hydrochloride with tert-butyl (R)- (2-aminopropyl)carbamate, subsequent Suzuki reaction with (3-(trifluoromethyl)-1H-pyrazol-4- yl)boronic acid, alkylation with 1- bromo-2-fluoroethane and acidic acidic Boc deprotection |
| 111 | 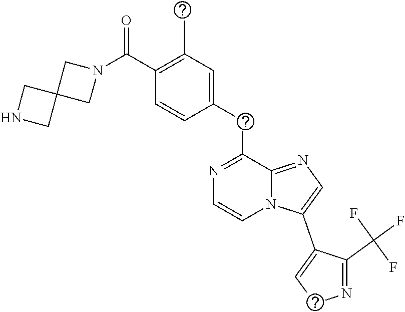 [SMILES: Cc1cc(Cc2nccn3c(C4=CCN=C4C(F)(F)F)cnc23)ccc1C(=O)N1CC2(CNC2)C1] | [2-chloro-4-[[3- [3- (trifluoromethyl)- 1H-pyrazol-4- yl]imidazo[1,2- a]pyrazin-8- yl]amino]phenyl]- (2,6- diazaspiro[3.3] heptan-2- yl)methanone | 502.9 | 501.4 | in analogy to the procedures for example 6: amide formation with 2-chloro-4-[[3-[3- (trifluoromethyl)-1H-pyrazol-4- yl]imidazo[1,2-a]pyrazin-8- yl]amino]benzoic acid and tert- butyl 2,6-diazaspiro[3.3]heptane- 2-carboxylate hemioxalate [CAS#1041026-71-4] and subsequent acidic Boc deprotection |
| 112 | 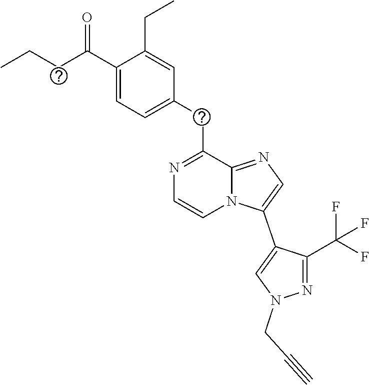 [SMILES: C#CCn1cc(-c2cnc3c(Cc4ccc(C(=O)CCC)c(CC)c4)nccn23)c(C(F)(F)F)n1] | N,2-diethyl-4-[[3- [1-prop-2-ynyl-3- (trifluoromethyl) pyrazol-4- yl]imidazo[1,2- a]pyrazin-8- yl]amino]benzamide | 481.5 | 482.4 | in analogy to the procedures for example 1: amide coupling of 2- ethyl-4-[[3-[3-(trifluoromethyl)- 1H-pyrazol-4-yl]imidazo[1,2- a]pyrazin-8-yl)amino]benzoic acid, hydrochloric acid with ethyl amine, subsequent Suzuki reaction with (3-(trifluoromethyl)-1H- pyrazol-4-yl)boronic acid, alkylation with 3-bromoprop-1- yne and acidic Boc deprotection |
| 113 | 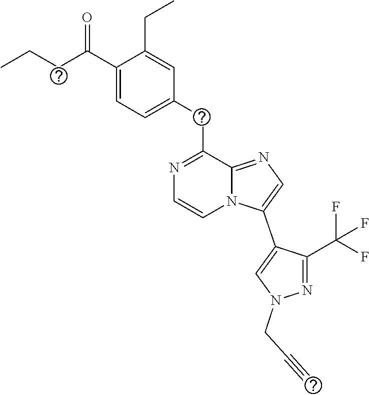 [SMILES: C#CCn1cc(-c2cnc3c(Cc4ccc(C(=O)CCC)c(CC)c4)nccn23)c(C(F)(F)F)n1] | 4-[[3-[1- (cyanomethyl)-3- (trifluoromethyl) pyrazol-4- yl]imidazo[1,2- a]pyrazin-8- yl]amino]-N,2- diethylbenzamide | 482.5 | 483.3 | in analogy to the procedures for example 1: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid with ethyl amine, subsequent Suzuki reaction with (3-(trifluoromethyl)-1H- pyrazol-4-yl)boronic acid, alkylation with 2- bromoacetonitrile and acidic Boc deprotection |
| 114 | 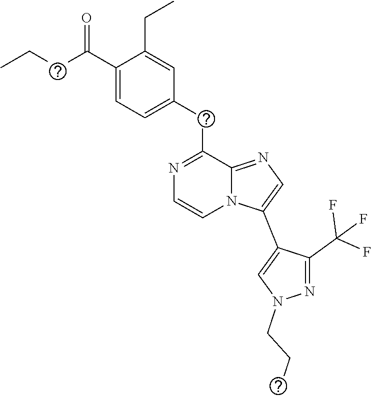 [SMILES: CCCC(=O)c1ccc(Cc2nccn3c(-c4cn(CCC)nc4C(F)(F)F)cnc23)cc1CC] | N,2-diethyl-4-[[3- [1-(2-fluoroethyl)- 3- (trifluoromethyl) pyrazol-4- yl]imidazo[1,2- a]pyrazin-8- yl]amino]benzamide | 489.5 | 490.4 | in analogy to the procedures for example 1: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid with ethyl amine, subsequent Suzuki reaction with (3-(trifluoromethyl)-1H- pyrazol-4-yl)boronic acid, alkylation with 1-bromo-2- fluoroethane and acidic Boc deprotection |
| 115 | 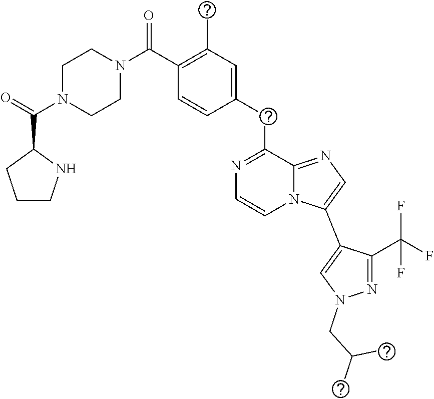 [SMILES: Cc1cc(Cc2nccn3c(-c4cn(CC(C)C)nc4C(F)(F)F)cnc23)ccc1C(=O)N1CCN(C(=O)[C@@H]2CCCN2)CC1] | [2-chloro-4-[[3- [1-(2,2- difluoroethyl)-3- (trifluoromethyl) pyrazol-4- yl]imidazo[1,2- a]pyrazin-8- yl]amino]phenyl]- [4-[(2S)- pyrrolidine-2- carbonyl]piperazin- 1-yl]methanone | 652.0 | 652.3 | in analogy to the procedures for example 6: amide formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (tert-butoxycarbonyl)-L-proline CAS [15761-39-4] and subsequent alkylation with 1,1-difluoro-2- iodoethane and acidic Boc deprotection |
| 116 | 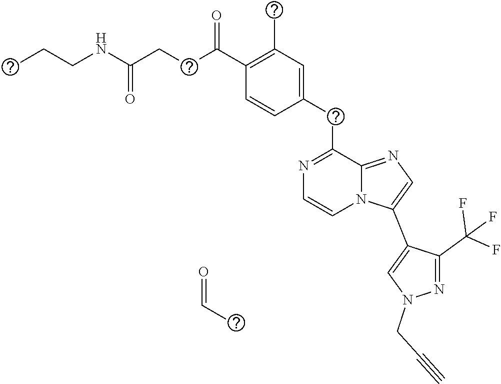 | N-[2-(2- aminoethylamino)- 2-oxoethyl]-2- chloro-4-[[3-[1- prop-2-ynyl-3- (trifluoromethyl) pyrazol-4- yl]imidazo[1,2- a]pyrazin-8- yl]amino]benzamide formate | 559.9 | 560 | in analogy to the procedures for example 8: alkylation of tert-butyl N-[2-[[2-[[2-chloro-4-[[3-[1-(2- fluoroethyl)-3- (trifluoromethyl)pyrazol-4- yl]imidazo[1,2-a]pyrazin-8- yl]amino]benzoyl]amino]acetyl] amino]ethyl]carbamate (example 8, step 4) with 3-bromoprop-1-yne and acidic Boc deprotection |
| 117 | 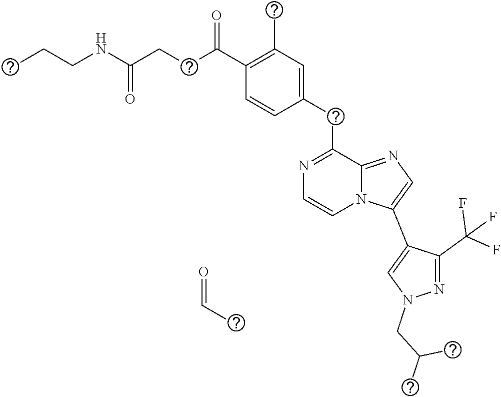 | N-[2-(2- aminoethylamino)- 2-oxoethyl]-2- chloro-4-[[3-[1- (2,2- difluoroethyl)-3- (trifluoromethyl) pyrazol-4- yl]imidazo[1,2- a]pyrazin-8- yl]amino]benzamide formate | 585.9 | 586 | in analogy to the procedures for example 8: alkylation of tert-butyl N-[2-[[2-[[2-chloro-4-[3-[1-(2- fluoroethyl)-3- (trifluoromethyl)pyrazol-4- yl]imidazo[1,2-a]pyrazin-8- yl]amino]benzoyl]amino]acetyl] amino]ethyl]carbamate (example 8, step 4) with 1,1-difluoro-2-iodo- ethane and acidic Boc deprotection |
| 118 | 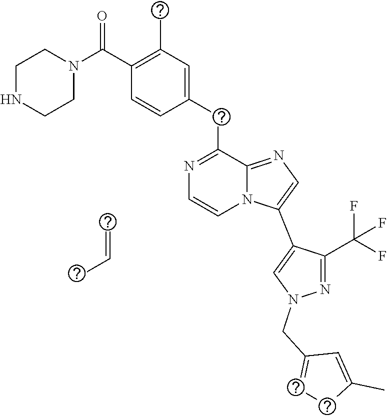 | [2-chloro-4-[[3- [1-[(5-methyl-1H- pyrazol-3- yl)methyl]-3- (trifluoromethyl) pyrazol-4- yl]imidazo[1,2- a]pyrazin-8- yl]amino]phenyl]- piperazin-1- ylmethanone formate | 585.0 | 585.2 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 3-(chloromethyl)-5-methyl- 1H-pyrazole and acidic Boc deprotection |
| 119 | 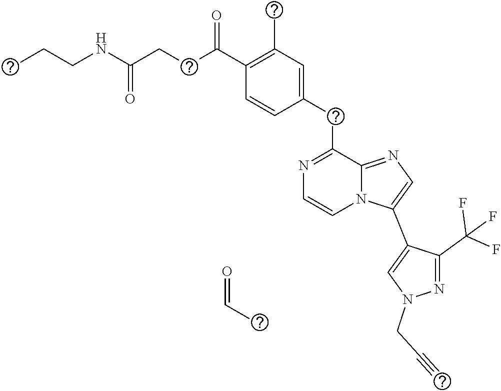 | N-[2-(2- aminoethylamino)- 2-oxoethyl]-2- chloro-4-[[3-[1- (cyanomethyl)-3- (trifluoromethyl) pyrazol-4- yl]imidazo[1,2- a]pyrazin-8- yl]amino]benzamide formate | 560.9 | 561.2 | in analogy to the procedures for example 8: alkylation of tert-butyl N-[2-[[2-[[2-chloro-4-[[3-[1-(2- fluoroethyl)-3- (trifluoromethyl)pyrazol-4- yl]imidazo[1,2-a]pyrazin-8- yl]amino]benzoyl]amino]acetyl] amino]ethyl]carbamate (example 8, step 4) with 2-bromoacetonitrile and acidic Boc deprotection |
| 120 | 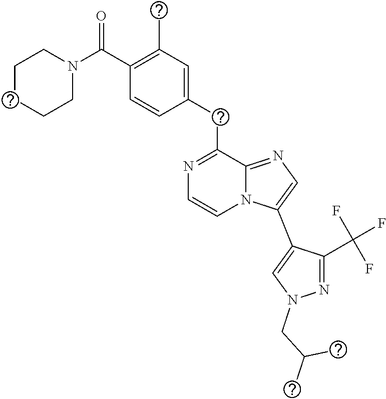 [SMILES: Cc1cc(Cc2nccn3c(-c4cn(CC(C)C)nc4C(F)(F)F)cnc23)ccc1C(=O)N1CCCCC1] | [2-chloro-4-[[3- [1-(2,2- difluoroethyl)-3- (trifluoromethyl) pyrazol-4- yl]imidazo[1,2- a]pyrazin-8- yl]amino]phenyl]- piperazin-1- ylmethanone | 554.9 | 553.4 [M − H]− | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 1,1-difluoro-2-iodo-ethane and acidic Boc deprotection |
| 121 | 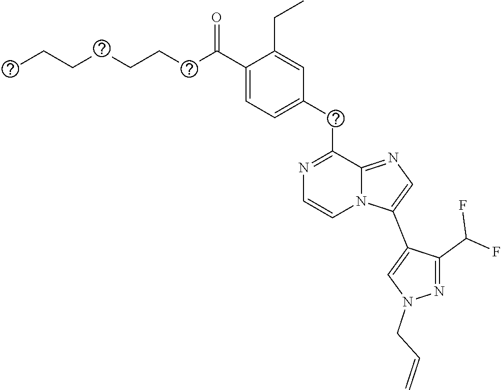 [SMILES: C=CCn1cc(-c2cnc3c(Cc4ccc(C(=O)CCCCCCC)c(CC)c4)nccn23)c(C(F)F)n1] | N-[2-(2- aminoethoxy)ethyl]- 4-[[3-[3- (difluoromethyl)- 1-prop-2- enylpyrazol-4- yl]imidazo[1,2- a]pyrazin-8- yl]amino]-2- ethylbenzamide | 524.6 | 525.4 | in analogy to the procedures for example 1: Suzuki reaction of tert- butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-(difluoromethyl)-4-(4,4,5,5- tetramethyl-1,3,2-dioxaborolan-2- yl)-1H-pyrazole, subsequent alkylation with 3-bromoprop-1-ene and acidic Boc deprotection |
| 122 | 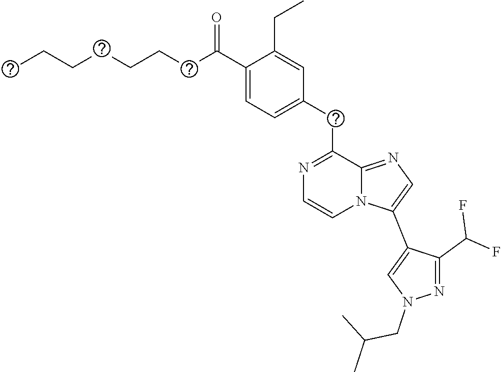 [SMILES: CCCCCCCC(=O)c1ccc(Cc2nccn3c(-c4cn(CC(C)C)nc4C(F)F)cnc23)cc1CC] | N-[2-(2- aminoethoxy)ethyl]- 4-[[3-[1-(2,2- difluoroethyl)-3- (difluoromethyl) pyrazol-4- yl]imidazo[1,2- a]pyrazin-8- yl]amino]-2- ethylbenzamide | 548.5 | 549.4 | in analogy to the procedures for example 1: Suzuki reaction of tert- butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-(difluoromethyl)-4-(4,4,5,5- tetramethyl-1,3,2-dioxaborolan-2- yl)-1H-pyrazole, subsequent alkylation with 1,1-difluoro-2- iodo-ethane and acidic Boc deprotection |
| 123 | 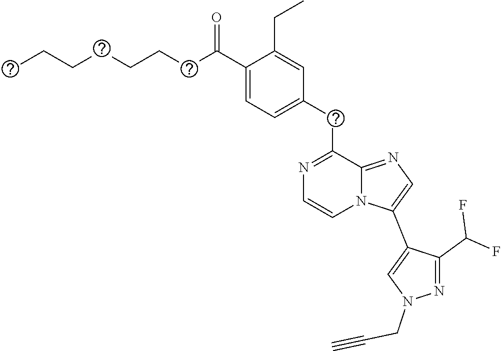 [SMILES: C#CCn1cc(-c2cnc3c(Cc4ccc(C(=O)CCCCCCC)c(CC)c4)nccn23)c(C(F)F)n1] | N-[2-(2- aminoethoxy)ethyl]- 4-[[3-[3- (difluoromethyl)- 1-prop-2- ynylpyrazol-4- yl]imidazo[1,2- a]pyrazin-8- yl]amino]-2- ethylbenzamide | 522.6 | 523.4 | in analogy to the procedures for example 1: Suzuki reaction of tert- butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-(difluoromethyl)-4-(4,4,5,5- tetramethyl-1,3,2-dioxaborolan-2- yl)-1H-pyrazole, subsequent alkylation with 3-bromoprop-1- yne and acidic Boc deprotection |
| 124 | 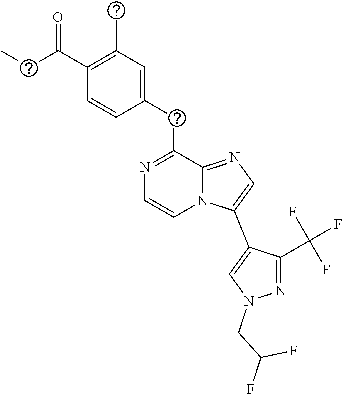 [SMILES: CCC(=O)c1ccc(Cc2nccn3c(-c4cn(CC(F)F)nc4C(F)(F)F)cnc23)cc1C] | 2-chloro-4-[[3- [1-(2,2- difluoroethyl)-3- (trifluoromethyl) pyrazol-4- yl]imidazo[1,2- a]pyrazin-8- yl]amino]-N- methylbenzamide | 499.8 | 500.3 | in analogy to the procedures for example 10: alkylation of 2- chloro-N-methyl-4-[[3-[3- (trifluoromethyl)-1H-pyrazol-4- yl]imidazo[1,2-a]pyrazin-8- yl]amino]benzamide (example 10, step 2) with 1,1-difluoro-2-iodo- ethane |
| 125 | 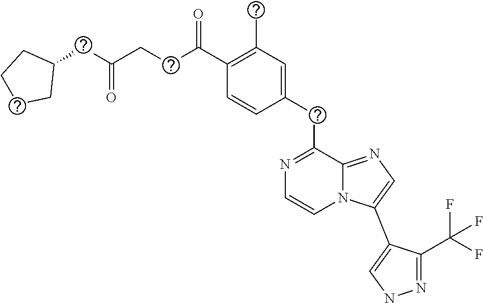 | 2-chloro-N-[2- oxo-2-[[(3S)- pyrrolidin-3- yl]amino]ethyl]-4- [[3-[3- (trifluoromethyl)- 1H-pyrazol-4- yl]imidazo[1,2- a]pyrazin-8- yl]amino]benzamide | 547.9 | 548.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with glycine ethyl ester hydrochloride, saponification under basic conditions, amide coupling with (S)-3-amino-1-N-BOC-pyrrolidine and acidic Boc deprotection |
| 126 | 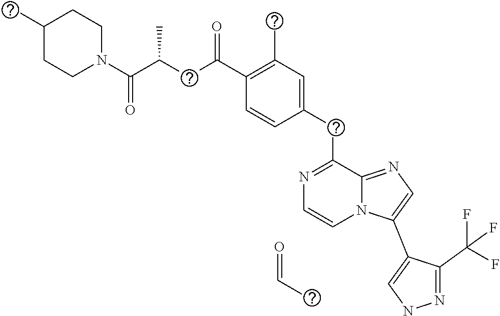 | N-[(2S)-1-(4- aminopiperidin-1- yl)-1-oxopropan- 2-yl]-2-chloro-4- [[3-[3- (trifluoromethyl)- 1H-pyrazol-4- yl]imidazo[1,2- a]pyrazin-8- yl]amino]benzamide formate | 576.0 | 576.1 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with L-alanine ethyl ester hydrochloride, saponification under basic conditions, amide coupling with 4- BOC-aminopiperidine and acidic Boc deprotection |
| 127 | 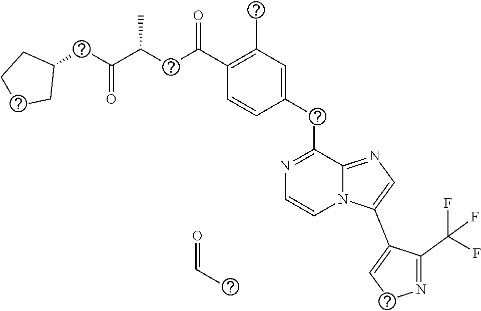 | 2-chloro-N-[(2S)- 1-oxo-1-[[(3S)- pyrrolidin-3- yl]amino]propan- 2-yl]-4-[[3-[3- (trifluoromethyl)- 1H-pyrazol-4- yl]imidazo[1,2- a]pyrazin-8- yl]amino]benzamide formate | 562.0 | 562 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with L-alanine ethyl ester hydrochloride, saponification under basic conditions, amide coupling with (S)-3-amino-1-N-BOC-pyrrolidine and acidic Boc deprotection |
| 128 | 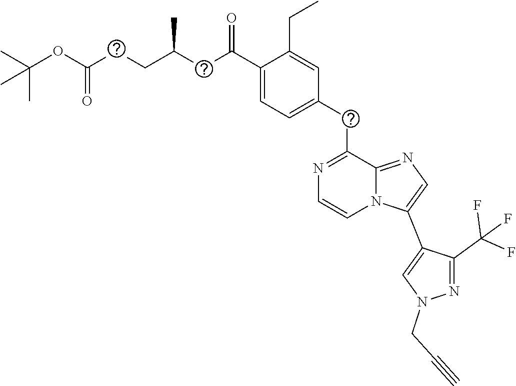 [SMILES: C#CCn1cc(-c2cnc3c(Cc4ccc(C(=O)C[C@H](C)CCC(=O)OC(C)(C)C)c(CC)c4)nccn23)c(C(F)(F)F)n1] | tert-butyl N- [(2R)-2-[[2-ethyl- 4-[[3-[1-prop-2- ynyl-3- (trifluoromethyl) pyrazol-4- yl]imidazo[1,2- a]pyrazin-8- yl]amino]benzoyl] amino]propyl] carbamate | 610.6 | 611.4 | in analogy to procedures described for example 3: amide coupling of 2-ethyl-4-[[3-[3-(trifluoromethyl)- 1H-pyrazol-4-yl]imidazo[1,2- a]pyrazin-8-yl]amino]benzoic acid, hydrochloric acid (example 3, step 3) with tert-butyl (R)-(2- aminopropyl)carbamate hydrochloride (commercially available) and alkylation with 3- bromoprop-1-yne |
| 129 | 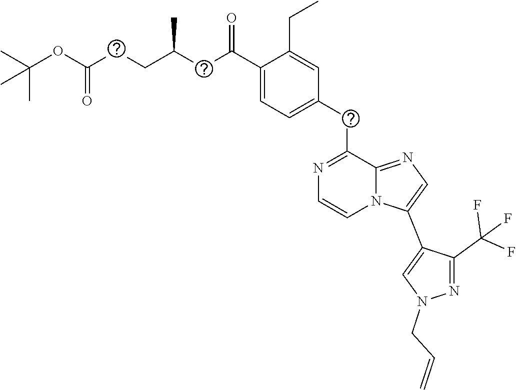 [SMILES: C=CCn1cc(-c2cnc3c(Cc4ccc(C(=O)C[C@H](C)CCC(=O)OC(C)(C)C)c(CC)c4)nccn23)c(C(F)(F)F)n1] | tert-butyl N- [(2R)-2-[[2-ethyl- 4-[[3-[1-prop-2- enyl-3- (trifluoromethyl) pyrazol-4- yl]imidazo[1,2- a]pyrazin-8- yl]amino]benzoyl] amino]propyl] carbamate | 612.7 | 613.4 | in analogy to procedures described for example 3: amide coupling of 2-ethyl-4-[[3-[3-(trifluoromethyl)- 1H-pyrazol-4-yl]imidazo[1,2- a]pyrazin-8-yl]amino]benzoic acid, hydrochloric acid (example 3, step 3) with tert-butyl (R)-(2- aminopropyl)carbamate hydrochloride (commercially available) and alkylation with 3- bromoprop-1-ene |
| 130 | 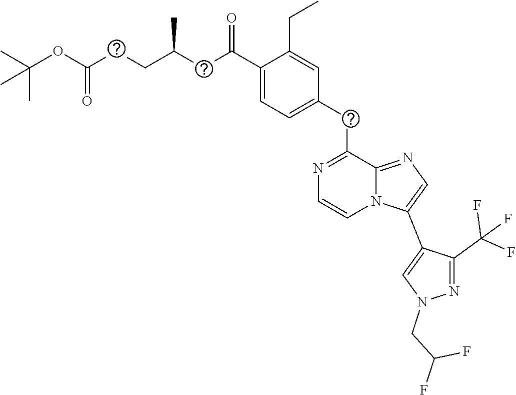 [SMILES: CCc1cc(Cc2nccn3c(-c4cn(CC(F)F)nc4C(F)(F)F)cnc23)ccc1C(=O)C[C@H](C)CCC(=O)OC(C)(C)C] | tert-butyl N- [(2R)-2-[[4-[[3-[1- (2,2- difluoroethyl)-3- (trifluoromethyl) pyrazol-4- yl]imidazo[1,2- a]pyrazin-8- yl]amino]-2- ethylbenzoyl] amino]propyl] carbamate | 636.6 | 637.4 | in analogy to procedures described for example 3: amide coupling of 2-ethyl-4-[[3-[3-(trifluoromethyl)- 1H-pyrazol-4-yl]imidazo[1,2- a]pyrazin-8-yl]amino]benzoic acid, hydrochloric acid (example 3, step 3) with tert-butyl (R)-(2- aminopropyl)carbamate hydrochloride (commercially available) and alkylation with 1,1- difluoro-2-iodo-ethane |
| 131 | 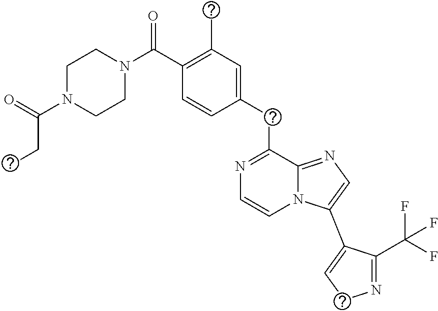 [SMILES: CCC(=O)N1CCN(C(=O)c2ccc(Cc3nccn4c(C5=CCN=C5C(F)(F)F)cnc34)cc2C)CC1] | 2-amino-1-[4-[2- chloro-4-[[3-[3- (trifluoromethyl)- 1H-pyrazol-4- yl]imidazo[1,2- a]pyrazin-8- yl]amino]benzoyl] piperazin-1- yl]ethanone | 547.9 | 548.3 | in analogy to procedures described for example 1, step 5: acidic Boc deprotection of tert-butyl N-[2-[4- [2-chloro-4-[[3-[3- (trifluoromethyl)-1H-pyrazol-4- yl]imidazo[1,2-a]pyrazin-8- yl]amino]benzoyl]piperazin-1-yl]- 2-oxo-ethyl]carbamate (example 6, step 6) |
| 132 | 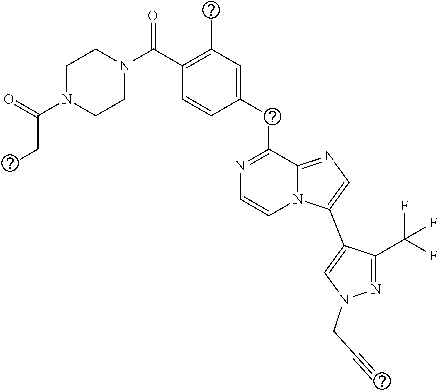 [SMILES: C#CCn1cc(-c2cnc3c(Cc4ccc(C(=O)N5CCN(C(=O)CC)CC5)c(C)c4)nccn23)c(C(F)(F)F)n1] | 2-[4-[8-[4-[4-(2- aminoacetyl) piperazine-1- carbonyl]-3- chloroanilino] imidazo[1,2- a]pyrazin-3-yl]-3- (trifluoromethyl) pyrazol-1- yl]acetonitrile | 587.0 | 587.3 | in analogy to the procedures for example 6: alkylation of tert-butyl N-[2-[4-[2-chloro-4-[[3-[3- (trifluoromethyl)-1H-pyrazol-4- yl]imidazo[1,2-a]pyrazin-8- yl]amino]benzoyl]piperazin-1-yl]- 2-oxo-ethyl]carbamate with 2- bromoacetonitrile and acidic Boc deprotection |
| 133 | 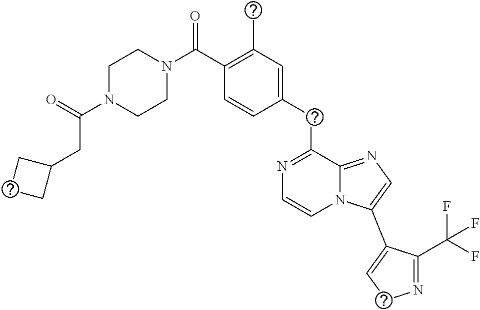 [SMILES: Cc1cc(Cc2nccn3c(C4=CCN=C4C(F)(F)F)cnc23)ccc1C(=O)N1CCN(C(=O)CC2CCC2)CC1] | 2-(azetidin-3-yl)- 1-[4-[2-chloro-4- [[3-[3- (trifluoromethyl)- 1H-pyrazol-4- yl]imidazo[1,2- a]pyrazin-8- yl]amino]benzoyl] piperazin-1- yl]ethanone | 588.0 | 588.3 | in analogy to the procedures for example 6: amide formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and 2-(1-(tert-butoxycarbonyl)azetidin- 3-yl)acetic acid [CAS#183062966] and acidic Boc deprotection |
| 134 | 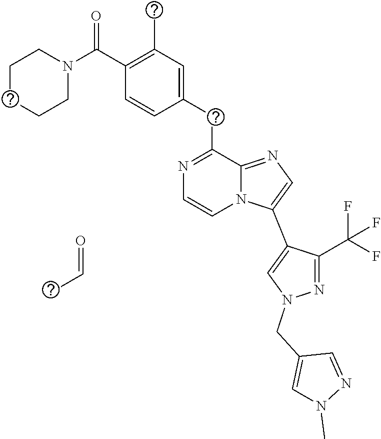 | [2-chloro-4-[[3- [1-[(1- methylpyrazol-4- yl)methyl]-3- (trifluoromethyl) pyrazol-4- yl]imidazo[1,2- a]pyrazin-8- yl]amino]phenyl]- piperazin-1- ylmethanone formate | 585.0 | 585 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 2-(1-(tert- butoxycarbonyl)azetidin-3- yl)acetic acid [CAS#183062966] and acidic Boc deprotection |
| 135 | 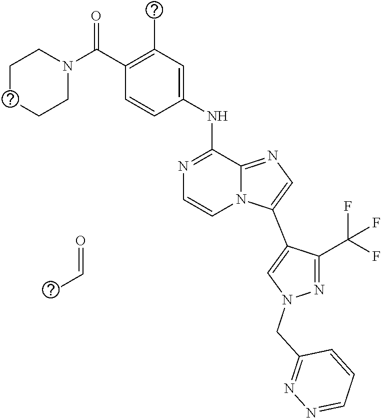 | [2-chloro-4-[[3- [1-(pyridazin-3- ylmethyl)-3- (trifluoromethyl) pyrazol-4- yl]imidazo[1,2- a]pyrazin-8- yl]amino]phenyl]- piperazin-1- ylmethanone formate | 583.0 | 583 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 4-(chloromethyl)pyrimidine and acidic Boc deprotection |
| 136 | 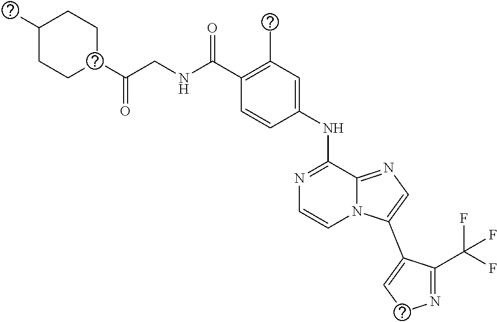 [SMILES: Cc1cc(Nc2nccn3c(C4=CCN=C4C(F)(F)F)cnc23)ccc1C(=O)NCC(=O)C1CCC(C)CC1] | N-[2-(4- aminopiperidin-1- yl)-2-oxoethyl]-2- chloro-4-[[3-[3- (trifluoromethyl)- 1H-pyrazol-4- yl]imidazo[1,2- a]pyrazin-8- yl]amino]benzamide | 562.0 | 562.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with glycine ethyl ester hydrochloride, saponification under basic conditions, amide coupling with 4- amino-1-boc-piperidine and acidic Boc deprotection |
| 137 | 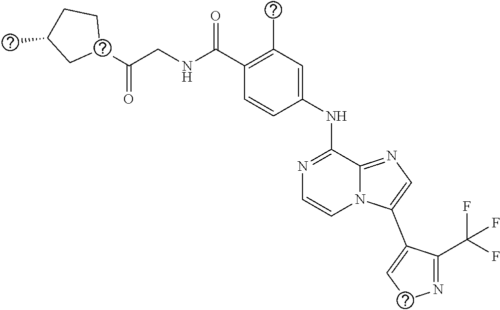 [SMILES: Cc1cc(Nc2nccn3c(C4=CCN=C4C(F)(F)F)cnc23)ccc1C(=O)NCC(=O)C1CC[C@@H](C)C1] | N-[2-[(3R)-3- aminopyrrolidin- 1-yl]-2-oxoethyl]- 2-chloro-4-[[3-[3- (trifluoromethyl)- 1H-pyrazol-4- yl]imidazo[1,2- a]pyrazin-8- yl]amino]benzamide | 547.9 | 548.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with glycine ethyl ester hydrochloride, saponification under basic conditions, amide coupling with (R)-3-(BOC-amino)pyrrolidine and acidic Boc deprotection |
| 138 | 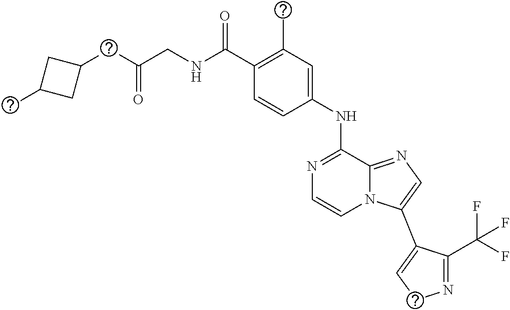 [SMILES: Cc1cc(Nc2nccn3c(C4=CCN=C4C(F)(F)F)cnc23)ccc1C(=O)NCC(=O)CC1CC(C)C1] | N-[2-[(3- aminocyclobutyl) amino]-2- oxoethyl]-2- chloro-4-[[3-[3- (trifluoromethyl)- 1H-pyrazol-4- yl]imidazo[1,2- a]pyrazin-8- yl]amino]benzamide | 547.9 | 548.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with glycine ethyl ester hydrochloride, saponification under basic conditions, amide coupling with tert-butyl 3- aminocyclobutylcarbamate and acidic Boc deprotection |
| 139 | 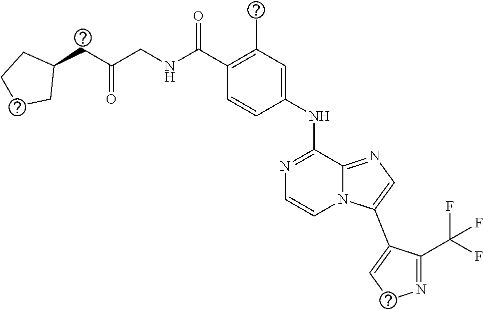 | 2-chloro-N-[2- oxo-2-[[(3R)- pyrrolidin-3- yl]amino]ethyl]-4- [[3-[3- (trifluoromethyl)- 1H-pyrazol-4- yl]imidazo[1,2- a]pyrazin-8- yl]amino]benzamide | 547.9 | 548.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with glycine ethyl ester hydrochloride, saponification under basic conditions, amide coupling with (R)-(+)-1-(BOC-3- aminopyrrolidine and acidic Boc deprotection |
| 140 | 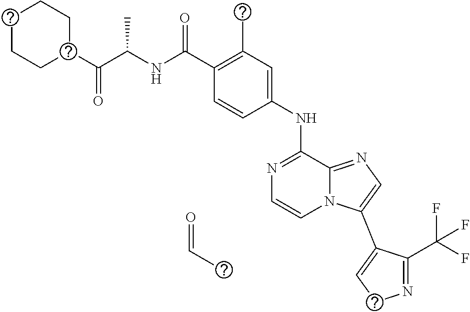 | 2-chloro-N-[(2S)- 1-oxo-1- piperazin-1- ylpropan-2-yl]-4- [[3-[3- (trifluoromethyl)- 1H-pyrazol-4- yl]imidazo[1,2- a]pyrazin-8- ylamino]benzamide formate | 562.0 | 562.3 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with L-alanine ethyl ester hydrochloride, saponification under basic conditions, amide coupling with 4- BOC-piperazine and acidic Boc deprotection |
| 141 | 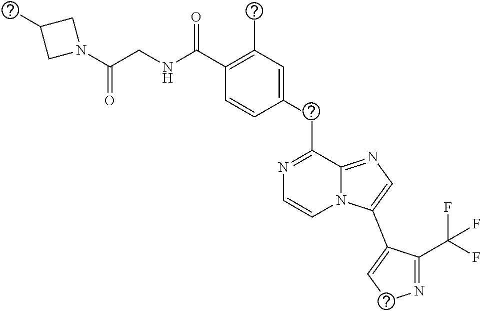 [SMILES: Cc1cc(Cc2nccn3c(C4=CCN=C4C(F)(F)F)cnc23)ccc1C(=O)NCC(=O)N1CC(C)C1] | N-[2-(3- aminoazetidin-1- yl)-2-oxoethyl]-2- chloro-4-[[3-[3- (trifluoromethyl)- 1H-pyrazol-4- yl]imidazo[1,2- a]pyrazin-8- yl]amino]benzamide | 533.9 | 534.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 3) with glycine ethyl ester hydrochloride, saponification under basic conditions, amide coupling with 1- BOC-3-(amino)azetidine and acidic Boc deprotection |
| 142 | 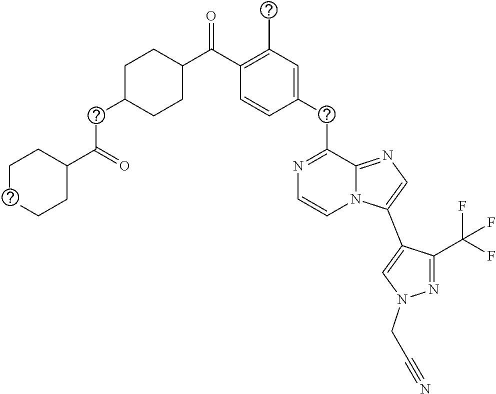 [SMILES: Cc1cc(Cc2nccn3c(-c4cn(CC#N)nc4C(F)(F)F)cnc23)ccc1C(=O)C1CCC(CC(=O)C2CCCCC2)CC1] | N-[1-[2-chloro-4- [[3-[1- (cyanomethyl)-3- (trifluoromethyl) pyrazol-4- yl]imidazo[1,2- a]pyrazin-8- yl]amino]benzoyl] piperidin-4- yl]piperidine-4- carboxamide | 655.1 | 655.5 | in analogy to procedures for example 6: amide coupling of (4- amino-1-piperidyl)-[2-chloro-4- [[3-[3-(trifluoromethyl)-1H- pyrazol-4-yl]imidazo[1,2- a]pyrazin-8- yl]amino]phenyl]methanone (example 11, step 2) with 1-(tert- butoxycarbonyl)piperidine-4- carboxylic acid [CAS#84358-13- 4], alkylation with 2- bromoacetonitrile and acidic Boc deprotection |
| 143 | 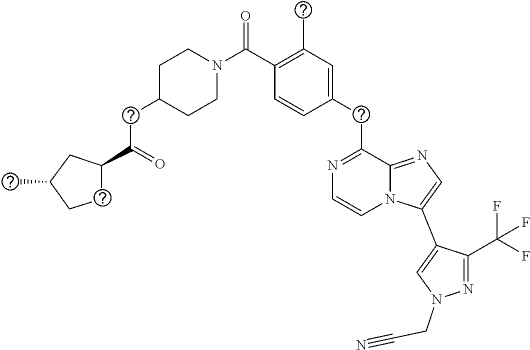 [SMILES: Cc1cc(Cc2nccn3c(-c4cn(CC#N)nc4C(F)(F)F)cnc23)ccc1C(=O)N1CCC(CC(=O)[C@H]2CC[C@H](C)C2)CC1] | (2S,4R)-N-[1-[2- chloro-4-[[3-[1- (cyanomethyl)-3- (trifluoromethyl) pyrazol-4- yl]imidazo[1,2- a]pyrazin-8- yl]amino]benzoyl] piperidin-4-yl]-4- hydroxypyrrolidine- 2-carboxamide | 657.1 | 657.5 | in analogy to procedures for example 6: amide coupling of (4- amino-1-piperidyl)-[2-chloro-4- [[3-[3-(trifluoromethyl)-1H- pyrazol-4-yl]imidazo[1,2- a]pyrazin-8- yl]amino]phenyl]methanone (example 11, step 2) with (2S,4R)- 1-(tert-butoxycarbonyl)-4- hydroxypyrrolidine-2-carboxylic acid [CAS#CIMP017207], alkylation with 2- bromoacetonitrile and acidic Boc deprotection |
| 144 | 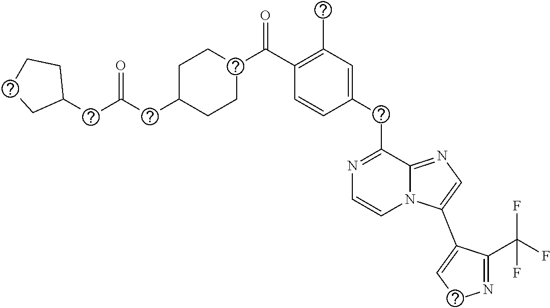 [SMILES: Cc1cc(Cc2nccn3c(C4=CCN=C4C(F)(F)F)cnc23)ccc1C(=O)C1CCC(CC(=O)CC2CCCC2)CC1] | 1-[1-[2-choro-4- [[3-[3- (trifluoromethyl)- 1H-pyrazol-4- yl]imidazo[1,2- a]pyrazin-8- yl]amino]benzoyl] piperidin-4-yl]-3- [(3S)-pyrrolidin- 3-yl]urea | 617.0 | 617.5 | in analogy to procedures for example 1: urea formation with (4- amino-1-piperidyl)-[2-chloro-4- [[3-[3-(trifluoromethyl)-1H- pyrazol-4-yl]imidazo[1,2- a]pyrazin-8- yl]amino]phenyl]methanone (example 11, step 2) and tert-butyl (S)-3-aminopyrrolidine-1- carboxylate and subsequent acidic Boc deprotection |
| 145 | 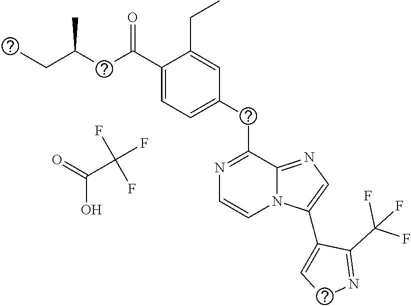 | N-[(2R)-1- aminopropan-2- yl]-2-ethyl-4-[[3- [3- (trifluoromethyl)- 1H-pyrazol-4- yl]imidazo[1,2- a]pyrazin-8- yl]amino]benzamide trifluoroacetate | 472.5 | 473.4 | in analogy to procedures for example 1: amide coupling of 2- ethyl-4-[(3-iodoimidazo[1,2- a]pyrazin-8-yl)amino]benzoic acid, hydrochloride with tert-butyl (R)- (2-aminopropyl)carbamate, subsequent Suzuki reaction with (3-(trifluoromethyl)-1H-pyrazol-4- yl)boronic acid and acidic Boc deprotection |
| 146 | 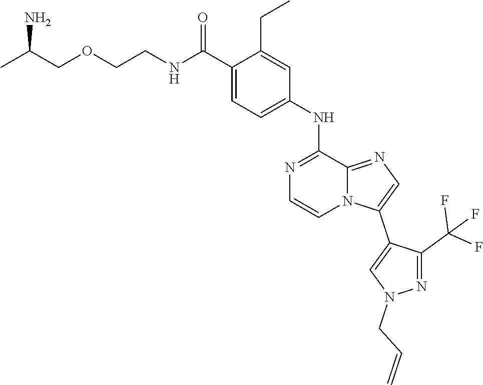 [SMILES: C=CCn1cc(-c2cnc3c(Nc4ccc(C(=O)NCCOC[C@@H](C)N)c(CC)c4)nccn23)c(C(F)(F)F)n1] | N-[2-[(2S)-2- aminopropoxy] ethyl]-2-ethyl-4-[[3- [1-prop-2-enyl-3- (trifluoromethyl) pyrazol-4- yl]imidazo[1,2- a]pyrazin-8- yl]amino]benzamide | 556.6 | 557.5 | in analogy to procedures for example 1: amide coupling of 2- ethyl-4-[(3-iodoimidazo[1,2- a]pyrazin-8-yl)amino]benzoic acid, hydrochloride with tert-butyl (S)- (1-(2-aminoethoxy)propan-2- yl)carbamate, subsequent Suzuki reaction with (3-(trifluoromethyl)- 1H-pyrazol-4-yl)boronic acid, alkylation with 3-bromoprop-1-ene and acidic Boc deprotection |
| 147 | 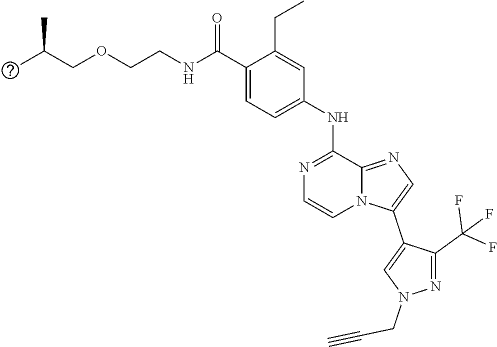 | N-[2-[(2S)-2- aminopropoxy] ethyl]-2-ethyl-4-[[3- [1-prop-2-ynyl-3- (trifluoromethyl) pyrazol-4- yl]imidazo[1,2- a]pyrazin-8- yl]amino]benzamide | 554.6 | 555.5 | in analogy to procedures for example 1: amide coupling of 2- ethyl-4-[(3-iodoimidazo[1,2- a]pyrazin-8-yl)amino]benzoic acid, hydrochloride with tert-butyl (S)- (1-(2-aminoethoxy)propan-2- yl)carbamate, subsequent Suzuki reaction with (3-(trifluoromethyl)- 1H-pyrazol-4-yl)boronic acid, alkylation with 3-bromoprop-1- yne and acidic Boc deprotection |
| 148 | 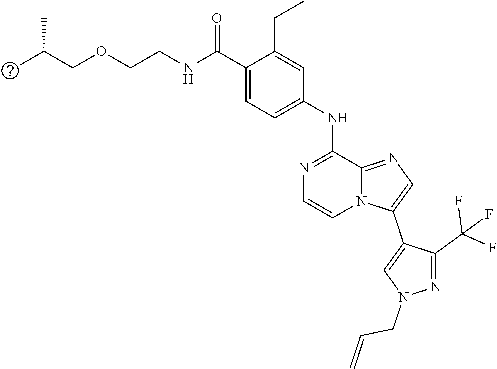 | N-[2-[(2R)-2- aminopropoxy] ethyl]-2-ethyl-4-[[3- [1-prop-2-enyl-3- (trifluoromethyl) pyrazol-4- yl]imidazo[1,2- a]pyrazin-8- yl]amino]benzamide | 556.6 | 557.5 | in analogy to procedures for example 1: amide coupling of 2- ethyl-4-[(3-iodoimidazo[1,2- a]pyrazin-8-yl)amino]benzoic acid, hydrochloride with tert-butyl (R)- (1-(2-aminoethoxy)propan-2- yl)carbamate, subsequent Suzuki reaction with (3-(trifluoromethyl)- 1H-pyrazol-4-yl)boronic acid, alkylation with 3-bromoprop-1-ene and acidic Boc deprotection |
| 149 | 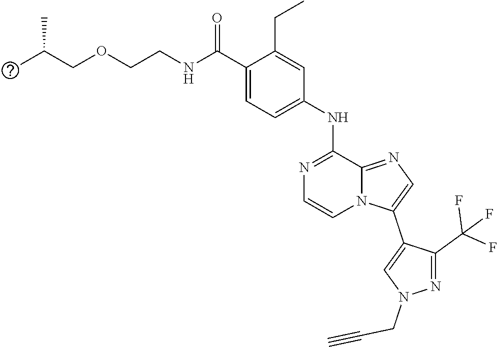 | N-[2-[(2R)-2- aminopropoxy] ethyl]-2-ethyl-4-[[3- [1-prop-2-ynyl-3- (trifluoromethyl) pyrazol-4- yl]imidazo[1,2- a]pyrazin-8- yl]amino]benzamide | 554.6 | 555.5 | in analogy to procedures for example 1: amide coupling of 2- ethyl-4-[(3-iodoimidazo[1,2- a]pyrazin-8-yl)amino]benzoic acid, hydrochloride with tert-butyl (R)- (1-(2-aminoethoxy)propan-2- yl)carbamate, subsequent Suzuki reaction with (3-(trifluoromethyl)- 1H-pyrazol-4-yl)boronic acid, alkylation with 3-bromoprop-1- yne and acidic Boc deprotection |
| 150 | 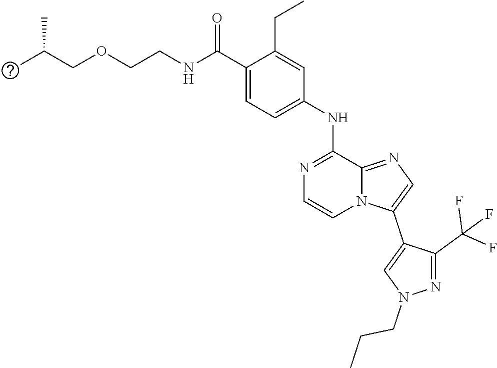 | N-[2-[(2R)-2- aminopropoxy] ethyl]-2-ethyl-4-[[3- [1-(2-fluoroethyl)- 3- (trifluoromethyl) pyrazol-4- yl]imidazo[1,2- a]pyrazin-8- yl]amino]benzamide | 562.6 | 563.5 | in analogy to procedures for example 1: amide coupling of 2- ethyl-4-[(3-iodoimidazo[1,2- a]pyrazin-8-yl)amino]benzoic acid, hydrochloride with tert-butyl (R)- (1-(2-aminoethoxy)propan-2- yl)carbamate, subsequent Suzuki reaction with (3-(trifluoromethyl)- 1H-pyrazol-4-yl)boronic acid, alkylation with 1-bromo-2- fluoroethane and acidic Boc deprotection |
| 151 | 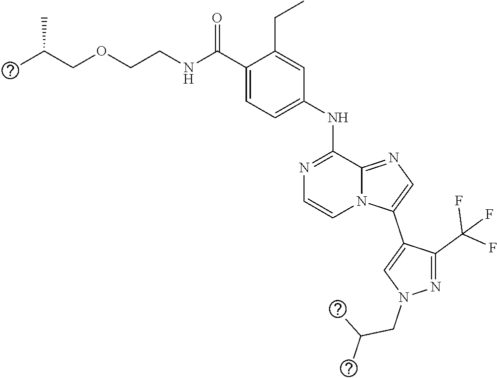 | N-[2-[(2R)-2- aminopropoxy] ethyl]-4-[[3-[1-(2,2- difluoroethyl)-3- (trifluoromethyl) pyrazol-4- yl]imidazo[1,2- a]pyrazin-8- yl]amino]-2- ethylbenzamide | 580.6 | 581.5 | in analogy to procedures for example 1: amide coupling of 2- ethyl-4-[(3-iodoimidazo[1,2- a]pyrazin-8-yl)amino]benzoic acid, hydrochloride with tert-butyl (R)- (1-(2-aminoethoxy)propan-2- yl)carbamate, subsequent Suzuki reaction with (3-(trifluoromethyl)- 1H-pyrazol-4-yl)boronic acid, alkylation with 1,1-difluoro-2- iodo-ethane and acidic Boc deprotection |
| 152 | 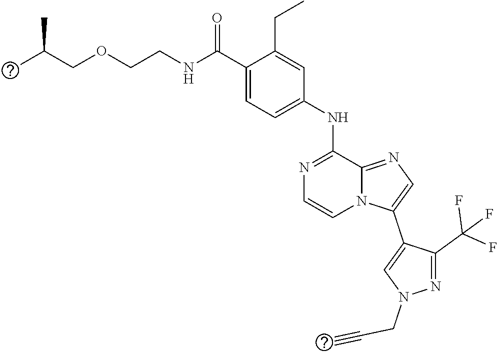 | N-[2-[(2S)-2- aminopropoxy] ethyl]-4-[[3-[1- (cyanomethyl)-3- (trifluoromethyl) pyrazol-4- yl]imidazo[1,2- a]pyrazin-8- yl]amino]-2- ethylbenzamide | 555.6 | 556.5 | in analogy to procedures for example 1: amide coupling of 2- ethyl-4-[(3-iodoimidazo[1,2- a]pyrazin-8-yl)amino]benzoic acid, hydrochloride with tert-butyl (S)- (1-(2-aminoethoxy)propan-2- yl)carbamate, subsequent Suzuki reaction with (3-(trifluoromethyl)- 1H-pyrazol-4-yl)boronic acid, alkylation with 2- bromoacetonitrile and acidic Boc deprotection |
| 153 | 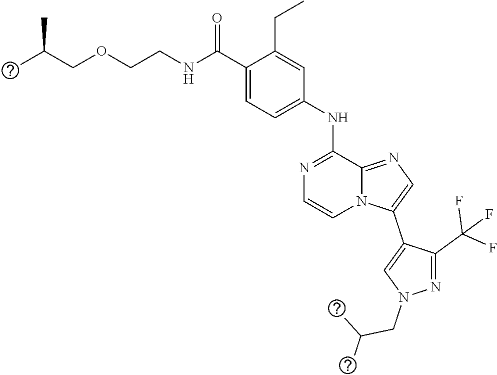 | N-[2-[(2S)-2- aminopropoxy] ethyl]-4-[[3-[1-(2,2- difluoroethyl)-3- (trifluoromethyl) pyrazol-4- yl]imidazo[1,2- a]pyrazin-8- yl]amino]-2- ethylbenzamide | 580.6 | 581.5 | in analogy to procedures for example 1: amide coupling of 2- ethyl-4-[(3-iodoimidazo[1,2- a]pyrazin-8-yl)amino]benzoic acid, hydrochloride with tert-butyl (S)- (1-(2-aminoethoxy)propan-2- yl)carbamate, subsequent Suzuki reaction with (3-(trifluoromethyl)- 1H-pyrazol-4-yl)boronic acid, alkylation with 1,1-difluoro-2- iodo-ethane and acidic Boc deprotection |
| 154 | 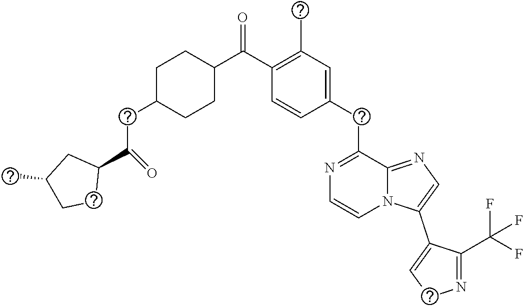 [SMILES: Cc1cc(Cc2nccn3c(C4=CCN=C4C(F)(F)F)cnc23)ccc1C(=O)C1CCC(CC(=O)[C@H]2CC[C@H](C)C2)CC1] | (2S,4R)-N-[1-[2- chloro-4-[[3-[3- (trifluoromethyl)- 1H-pyrazol-4- yl]imidazo[1,2- a]pyrazin-8- yl]amino]benzoyl] piperidin-4-yl]-4- hydroxypyrrolidine- 2-carboxamide | 618.0 | 618.4 | in analogy to procedures for example 6: amide coupling of (4- amino-1-piperidyl)-[2-chloro-4- [[3-[3-(trifluoromethyl)-1H- pyrazol-4-yl]imidazo[1,2- a]pyrazin-8- yl]amino]phenyl]methanone (example 11, step 2) with (2S,4R)- 1-(tert-butoxycarbonyl)-4- hydroxypyrrolidine-2-carboxylic acid and acidic Boc deprotection |
| 155 | 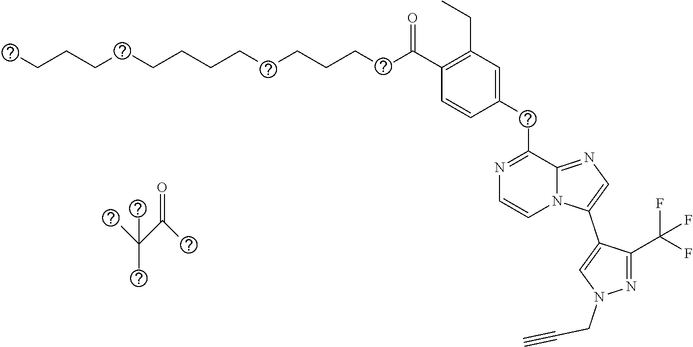 | N-[3-[4-(3- aminopropylamino) butylamino]propyl]- 2-ethyl-4-[[3- [1-prop-2-ynyl-3- (trifluoromethyl) pyrazol-4- yl]imidazo[1,2- a]pyrazin-8- yl]amino]benzamide trifluoroacetate | 638.7 | 639.5 | In analogy to the procedure described for example 2, step 2 the title compound was prepared from tert-butyl N-[3-(tert- butoxycarbonylamino)propyl]-N- [4-[tert-butoxycarbonyl-[3-[[2- ethyl-4-[[3-[3-(trifluoromethyl)- 1H-pyrazol-4-yl]imidazo[1,2- a]pyrazin-8- yl]amino]benzoyl]amino]propyl] amino]butyl]carbamate and 3- bromoprop-1-yne and subsequent acidic Boc deprotection |
| 156 | 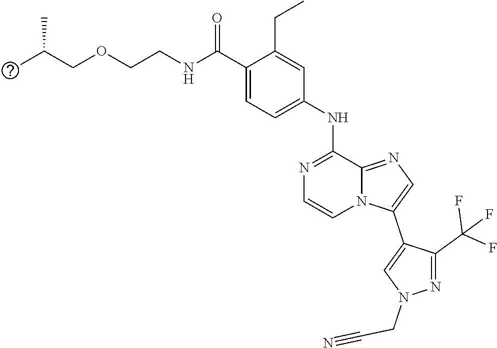 | N-[2-[(2R)-2- aminopropoxy] ethyl]-4-[[3-[1- (cyanomethyl)-3- (trifluoromethyl) pyrazol-4- yl]imidazo[1,2- a]pyrazin-8- yl]amino]-2- ethylbenzamide | 555.6 | 556.5 | in analogy to procedures for example 1: amide coupling of 2- ethyl-4-[(3-iodoimidazo[1,2- a]pyrazin-8-yl)amino]benzoic acid, hydrochloride with tert-butyl (R)- (1-(2-aminoethoxy)propan-2- yl)carbamate, subsequent Suzuki reaction with (3-(trifluoromethyl)- 1H-pyrazol-4-yl)boronic acid, alkylation with 2- bromoacetonitrile and acidic Boc deprotection |
| 157 | 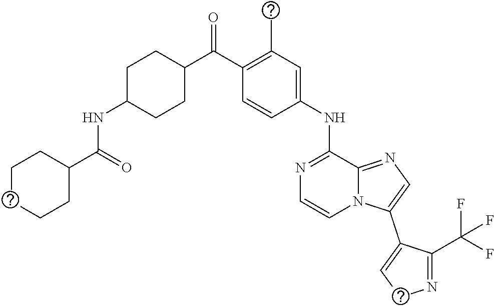 [SMILES: Cc1cc(Nc2nccn3c(C4=CCN=C4C(F)(F)F)cnc23)ccc1C(=O)C1CCC(NC(=O)C2CCCCC2)CC1] | N-[1-[2-chloro-4- [[3-[3- (trifluoromethyl)- 1H-pyrazol-4- yl]imidazo[1,2- a]pyrazin-8- yl]amino]benzoyl] piperidin-4- yl]piperidine-4- carboxamide | 616.0 | 616.5 | in analogy to procedures for example 6: amide coupling of (4- amino-1-piperidyl)-[2-chloro-4- [[3-[3-(trifluoromethyl)-1H- pyrazol-4-yl]imidazo[1,2- a]pyrazin-8- yl]amino]phenyl]methanone (example 11, step 2) with 1-(tert- butoxycarbonyl)piperidine-4- carboxylic acid [CAS#84358-13- 4] and acidic Boc deprotection |
| 158 | 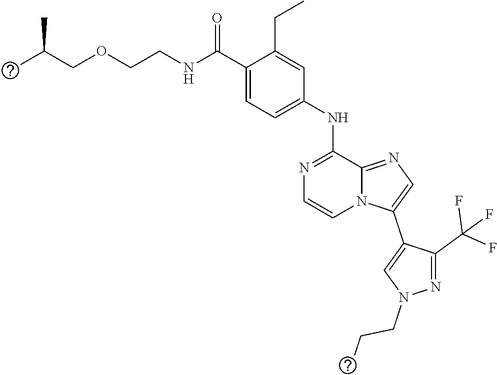 | N-[2-[(2S)-2- aminopropoxy] ethyl]-2-ethyl-4-[[3- [1-(2-fluoroethyl)- 3- (trifluoromethyl) pyrazol-4- yl]imidazo[1,2- a]pyrazin-8- yl]amino]benzamide | 562.6 | 563.5 | in analogy to procedures for example 1: amide coupling of 2- ethyl-4-[(3-iodoimidazo[1,2- a]pyrazin-8-yl)amino]benzoic acid, hydrochloride with tert-butyl (S)- (1-(2-aminoethoxy)propan-2- yl)carbamate, subsequent Suzuki reaction with (3-(trifluoromethyl)- 1H-pyrazol-4-yl)boronic acid, alkylation with 1-bromo-2- fluoroethane and acidic Boc deprotection |
| 159 | 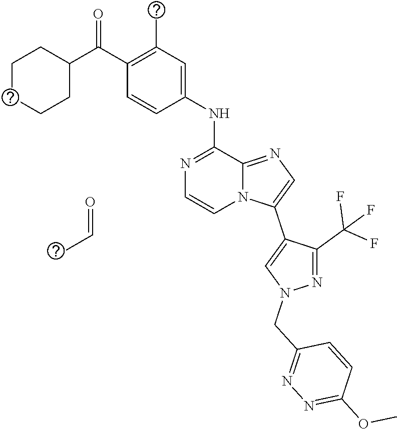 | [2-chloro-4-[[3- [1-[(6- methoxypyridazin- 3-yl)methyl]-3- (trifluoromethyl) pyrazol-4- yl]imidazo[1,2- a]pyrazin-8- yl]amino]phenyl]- piperazin-1- ylmethanone formate | 613.0 | 613.1 | in analogy to procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 3-(chloromethyl)-6-methoxy- pyridazine and acidic Boc deprotection |
| 160 | 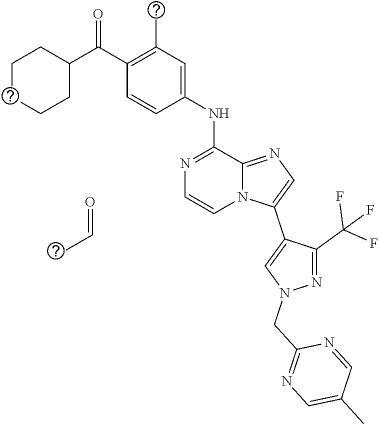 | [2-chloro-4-[[3- [1-[(5- methylpyrimidin- 2-yl)methyl]-3- (trifluoromethyl) pyrazol-4- yl]imidazo[1,2- a]pyrazin-8- yl]amino]phenyl]- piperazin-1- ylmethanone formate | 597.0 | 597.2 | in analogy to procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 2-(chloromethyl)-5-methyl- pyrimidine and acidic Boc deprotection |
| 161 | 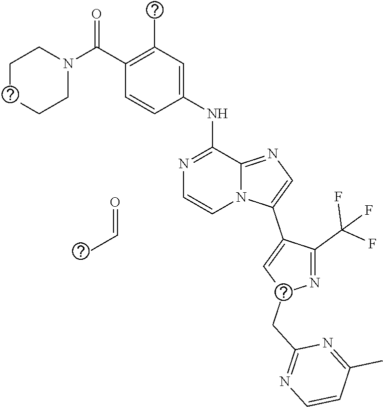 | [2-chloro-4-[[3- [1-[(4- methylpyrimidin- 2-yl)methyl]-3- (trifluoromethyl) pyrazol-4- yl]imidazo[1,2- a]pyrazin-8- yl]amino]phenyl]- piperazin-1- ylmethanone formate | 597.0 | 597.2 | in analogy to procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 2-(chloromethyl)-4-methyl- pyrimidine and acidic Boc deprotection |
| 162 | 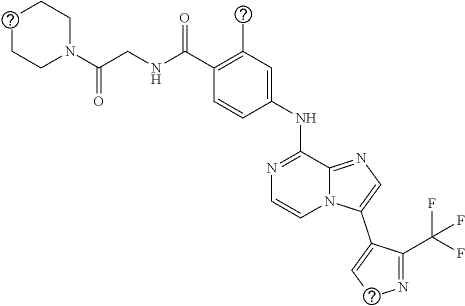 [SMILES: Cc1cc(Nc2nccn3c(C4=CCN=C4C(F)(F)F)cnc23)ccc1C(=O)NCC(=O)N1CCCCC1] | 2-chloro-N-(2- oxo-2-piperazin- 1-ylethyl)-4-[[3- [3- (trifluoromethyl)- 1H-pyrazol-4- yl]imidazo[1,2- a]pyrazin-8- yl]amino]benzamide | 547.9 | 548.2 | in analogy to the procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 2) with glycine ethyl ester hydrochloride, saponification under basic conditions, amide coupling with 1- BOC-piperazine and acidic Boc deprotection |
| 163 | 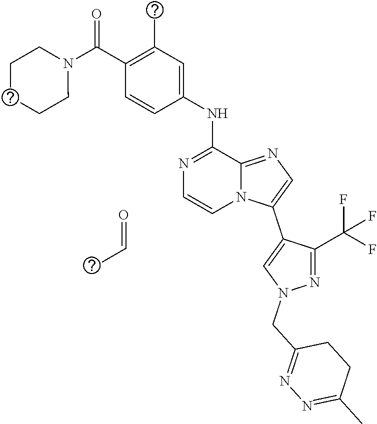 | [2-chloro-4-[[3- [1-[(6- methylpyridazin- 3-yl)methyl]-3- (trifluoromethyl) pyrazol-4- yl]imidazo[1,2- a]pyrazin-8- yl]amino]phenyl]- piperazin-1- ylmethanone formate | 597.0 | 597 | in analogy to procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 3-(chloromethyl)-6-methyl- pyridazine and acidic Boc deprotection |
| 164 | 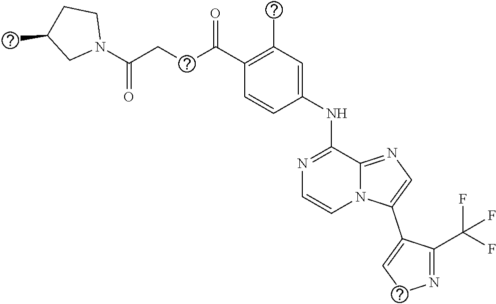 [SMILES: Cc1cc(Nc2nccn3c(C4=CCN=C4C(F)(F)F)cnc23)ccc1C(=O)CCC(=O)N1CC[C@H](C)C1] | N-[2-[(3S)-3- aminopyrrolidin- 1-yl]-2-oxoethyl]- 2-chloro-4-[[3-[3- (trifluoromethyl)- 1H-pyrazol-4- yl]imidazo[1,2- a]pyrazin-8- yl]amino]benzamide | 547.9 | 548.2 | in analogy to procedures for example 8: chloro-4-[[3-[3-(trifluoromethyl)- 1H-pyrazol-4-yl]imidazo[1,2- a]pyrazin-8-yl]amino]benzoic acid (example 8, step 2) with glycine ethyl ester hydrochloride, saponification under basic conditions, amide coupling with (S)-3-(BOC-amino)pyrrolidine and acidic Boc deprotection |
| 165 | 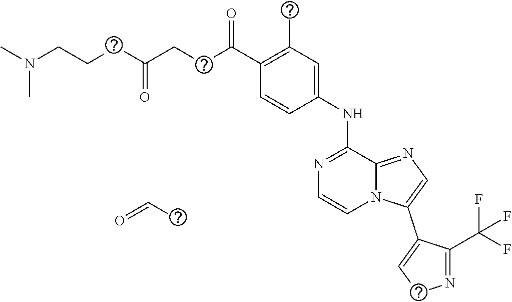 | 2-chloro-N-[2-[2- (dimethylamino) ethylamino]-2- oxoethyl]-4-[[3- [3- (trifluoromethyl)- 1H-pyrazol-4- yl]imidazo[1,2- a]pyrazin-8- yl]amino]benzamide formate | 549.9 | 550.2 | in analogy to procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 2) with glycine ethyl ester hydrochloride, saponification under basic conditions, amide coupling with N,N-dimethylethylenediamine |
| 166 | 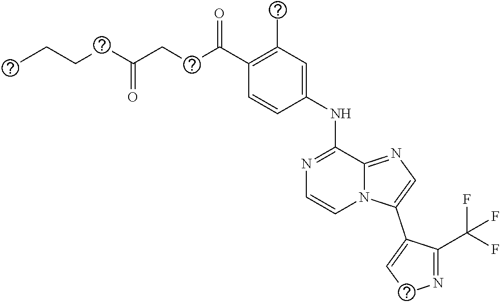 [SMILES: CCCCC(=O)CCC(=O)c1ccc(Nc2nccn3c(C4=CCN=C4C(F)(F)F)cnc23)cc1C] | N-[2-(2- aminoethylamino)- 2-oxoethyl]-2- chloro-4-[[3-[3- (trifluoromethyl)- 1H-pyrazol-4- yl]imidazo[1,2- a]pyrazin-8- yl]amino]benzamide | 521.9 | 522.2 | in analogy to procedures for example 8: amide formation of 2- chloro-4-[[3-[3-(trifluoromethyl)- 1H-pyrazol-4-yl]imidazo[1,2- a]pyrazin-8-yl]amino]benzoic acid (example 8, step 2) with glycine ethyl ester hydrochloride, saponification under basic conditions, amide coupling with N-BOC-ethylenediamine and acidic Boc deprotection |
| 167 | 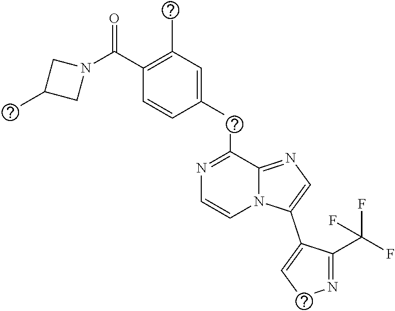 [SMILES: Cc1cc(Cc2nccn3c(C4=CCN=C4C(F)(F)F)cnc23)ccc1C(=O)N1CC(C)C1] | (3-aminoazetidin- 1-yl)-[2-chloro-4- [[3-[3- (trifluoromethyl)- 1H-pyrazol-4- yl]pyrazol-4- yl]imidazo[1,2- a]pyrazin-8- yl]amino]phenyl] methanone | 476.8 | 477.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl azetidin-3-ylcarbamate and subsequent acidic Boc deprotection |
| 168 | 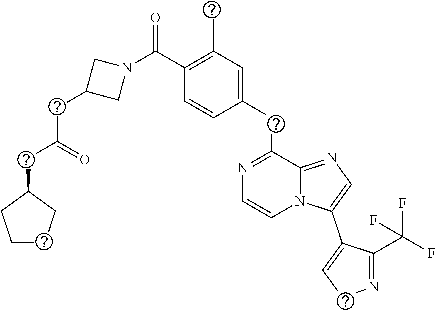 | 1-[1-[2-chloro-4- [[3-[3- (trifluoromethyl)- 1H-pyrazol-4- yl]imidazo[1,2- a]pyrazin-8- yl]amino]benzoyl] azetidin-3-yl]-3- [(3R)-pyrrolidin- 3-yl]urea | 589.0 | 587.5 [M − H] | in analogy to procedures for example 1: urea formation with N- [2-(3-aminoazetidin-1-yl)-2- oxoethyl]-2-chloro-4-[[3-[3- (trifluoromethyl)-1H-pyrazol-4- yl]imidazo[1,2-a]pyrazin-8- yl]amino]benzamide (example 167) and tert-butyl tert- butyl (R)-3-aminopyrrolidine-1- carboxylate [CAS#147081-49-0] and subsequent acidic Boc deprotection |
| 169 | 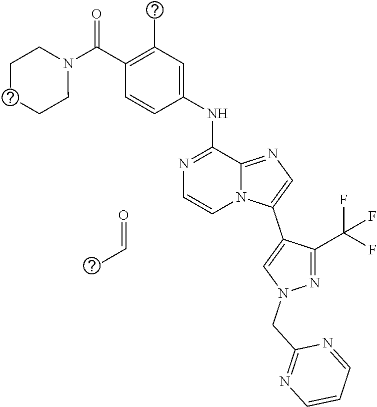 | [2-chloro-4-[[3- [1-(pyrimidin-2- ylmethyl)-3- (trifluoromethyl) pyrazol-4- yl]imidazo[1,2- a]pyrazin-8- yl]amino]phenyl]- piperazin-1- ylmethanone formate | 583.0 | 583.3 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 2-(chloromethyl)pyrimidine and acidic Boc deprotection |
| 170 | 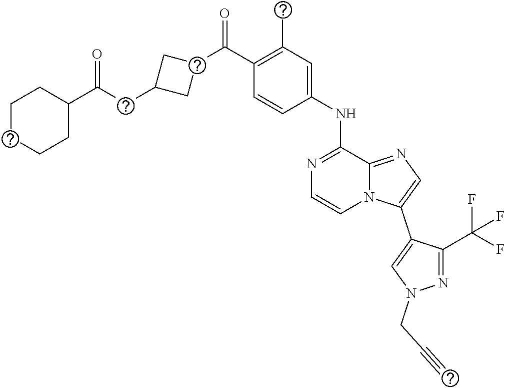 [SMILES: C#CCn1cc(-c2cnc3c(Nc4ccc(C(=O)C5CC(CC(=O)C6CCCCC6)C5)c(C)c4)nccn23)c(C(F)(F)F)n1] | N-[1-[2-chloro-4- [[3-[1- (cyanomethyl)-3- (trifluoromethyl) pyrazol-4- yl]imidazo[1,2- a]pyrazin-8- yl]amino]benzoyl] azetidin-3- yl]piperidine-4- carboxamide | 627.0 | 625.6 [M − H]− | in analogy to procedures for example 1: amide formation with N-[2-(3-aminoazetidin-1-yl)-2- oxoethyl]-2-chloro-4-[[3-[3- (trifluoromethyl)-1H-pyrazol-4- yl]imidazo[1,2-a]pyrazin-8- yl]amino]benzamide (example 167) and 1-(tert- butoxycarbonyl)piperidine-4- carboxylic acid [CAS#84358-13- 4], alkylation with 2- bromoacetonitrile [CAS#590-17-0] and subsequent acidic Boc deprotection |
| 171 | 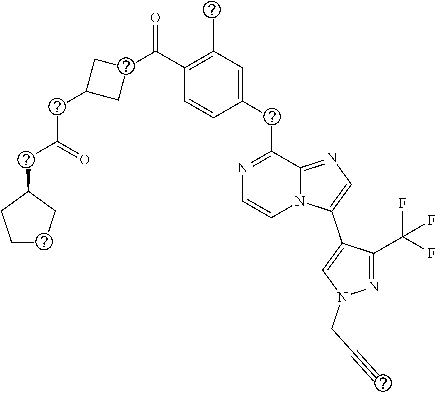 | 1-[1-[2-chloro-4- [[3-[1- (cyanomethyl)-3- (trifluoromethyl) pyrazol-4- yl]imidazo[1,2- a]pyrazin-8- yl]amino]benzoyl] azetidin-3-yl]-3- [(3R)-pyrrolidin- 3-yl]urea | 628.0 | 628.4 | in analogy to procedures for example 1: urea formation with N-[2-(3-aminoazetidin-1-yl)- 2-oxoethyl]-2-chloro-4-[[3-[3- (trifluoromethyl)-1H-pyrazol-4- yl]imidazo[1,2-a]pyrazin-8- yl]amino]benzamide (example 167) and tert-butyl (R)- 3-aminopyrrolidine-1-carboxylate [CAS#147081-49-0], alkylation with 2-bromoacetonitrile [CAS#590-17-0] and subsequent acidic Boc deprotection |
| 172 | 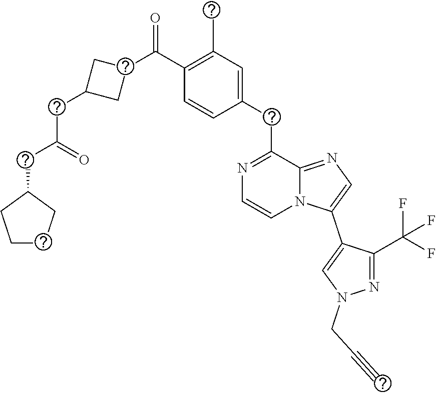 | 1-[1-[2-chloro-4- [[3-[1- (cyanomethyl)-3- (trifluoromethyl) pyrazol-4- yl]imidazo[1,2- a]pyrazin-8- yl]amino]benzoyl] azetidin-3-yl]-3- [(3S)-pyrrolidin- 3-yl]urea | 628.0 | 628.5 | in analogy to procedures for example 1: urea formation with N-[2-(3-aminoazetidin-1-yl)- 2-oxoethyl]-2-chloro-4-[[3-[3- (trifluoromethyl)-1H-pyrazol-4- yl]imidazo[1,2-a]pyrazin-8- yl]amino]benzamide (example 167) and tert-butyl (S)-3- aminopyrrolidine-1-carboxylate [CAS#147081-49-0], alkylation with 2-bromoacetonitrile [CAS#590-17-0] and subsequent acidic Boc deprotection |
| 173 | 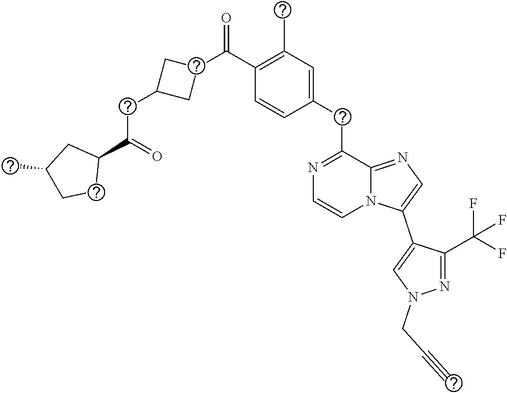 [SMILES: C#CCn1cc(-c2cnc3c(Cc4ccc(C(=O)C5CC(CC(=O)[C@H]6CC[C@H](C)C6)C5)c(C)c4)nccn23)c(C(F)(F)F)n1] | (2S,4R)-N-[1-[2- chloro-4-[[3-[1- (cyanomethyl)-3- (trifluoromethyl) pyrazol-4- yl]imidazo[1,2- a]pyrazin-8- yl]amino]benzoyl] azetidin-3-yl]-4- hydroxypyrrolidine- 2-carboxamide | 629.0 | 627.5 [M − H]− | in analogy to procedures for example 1: amide formation with N-[2-(3-aminoazetidin-1-yl)-2- oxoethyl]-2-chloro-4-[[3-[3- (trifluoromethyl)-1H-pyrazol-4- yl]imidazo[1,2-a]pyrazin-8- yl]amino]benzamide (example 167) and (2S,4R)-1-(tert- butoxycarbonyl)-4- hydroxypyrrolidine-2-carboxylic acid [CAS#13726-69-7], alkylation with 2- bromoacetonitrile [CAS#590-17-0] and subsequent acidic Boc deprotection |
| 174 | 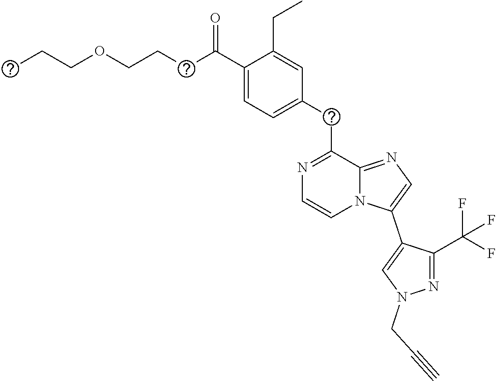 [SMILES: C#CCn1cc(-c2cnc3c(Cc4ccc(C(=O)CCCOCCC)c(CC)c4)nccn23)c(C(F)(F)F)n1] | N-[2-(2- aminoethoxy)ethyl]- 2-ethyl-4-[[3- [1-prop-2-ynyl-3- (trifluoromethyl) pyrazol-4- yl]imidazo[1,2- a]pyrazin-8- yl]amino]benzamide | 540.5 | 541.3 | in analogy to the procedure for example 2 from tert-butyl N-[2-[2- [[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3-bromoprop-1- yne and subsequent acidic cleavage of the Boc group |
| 175 | 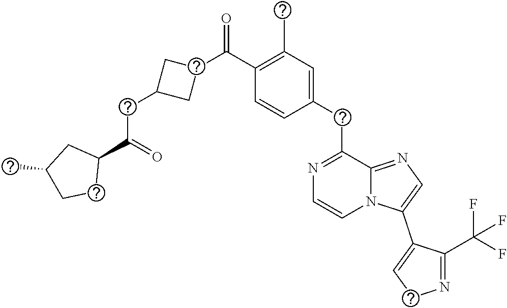 [SMILES: Cc1cc(Cc2nccn3c(C4=CCN=C4C(F)(F)F)cnc23)ccc1C(=O)C1CC(CC(=O)[C@H]2CC[C@H](C)C2)C1] | (2S,4R)-N-[1-[2- chloro-4-[[3-[3- (trifluoromethyl)- 1H-pyrazol-4- yl]imidazo[1,2- a]pyrazin-8- yl]amino]benzoyl] azetidin-3-yl]-4- hydroxypyrrolidine- 2-carboxamide | 590.0 | 588.5 [M − H]− | in analogy to the procedures for example 1: amide formation with N-[2-(3-aminoazetidin-1-yl)-2- oxoethyl]-2-chloro-4-[[3-[3- (trifluoromethyl)-1H-pyrazol-4- yl]imidazo[1,2-a]pyrazin-8- yl]amino]benzamide (example 167) and (2S,4R)-1-(tert- butoxycarbonyl)-4- hydroxypyrrolidine-2-carboxylic acid [CAS#13726-69-7] and subsequent acidic Boc deprotection |
| 176 | 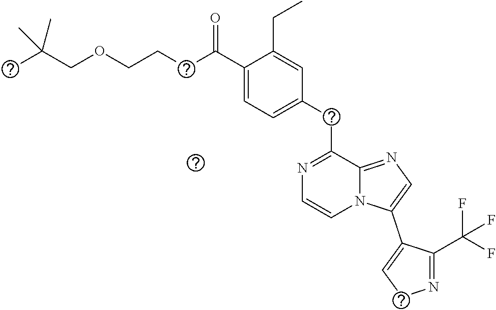 [SMILES: CCc1cc(Cc2nccn3c(C4=CCN=C4C(F)(F)F)cnc23)ccc1C(=O)CCCOCC(C)(C)C] | N-[2-(2-amino-2- methylpropoxy) ethyl]-2-ethyl-4-[[3- [3- (trifluoromethyl)- 1H-pyrazol-4- yl]imidazo[1,2- a]pyrazin-8- yl]amino]benzamide hydrochloride | 530.6 | 531.4 | in analogy to the procedures for example 1: amide coupling of 2- ethyl-4-[(3-iodoimidazo[1,2- a]pyrazin-8-yl)amino]benzoic acid, hydrochloride with tert-butyl (1- (2-aminoethoxy)-2-methylpropan- 2-yl)carbamate, subsequent Suzuki reaction with (3-(trifluoromethyl)- 1H-pyrazol-4-yl)boronic acid and acidic Boc deprotection |
| 177 | 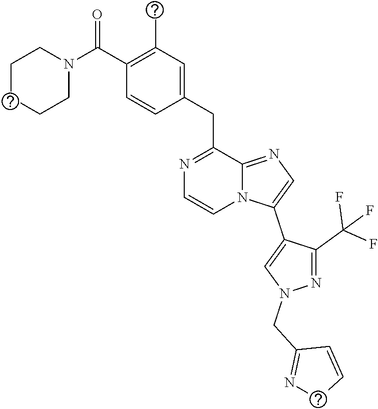 [SMILES: Cc1cc(Cc2nccn3c(-c4cn(CC5=NCC=C5)nc4C(F)(F)F)cnc23)ccc1C(=O)N1CCCCC1] | [2-chloro-4-[[3- [1-(1H-pyrazol-3- ylmethyl)-3- (trifluoromethyl) pyrazol-4- yl]imidazo[1,2- a]pyrazin-8- yl]amino]phenyl]- piperazin-1- ylmethanone | 571.0 | 571.2 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 3-(chloromethyl)-1H- pyrazole and acidic Boc deprotection |
| 178 | 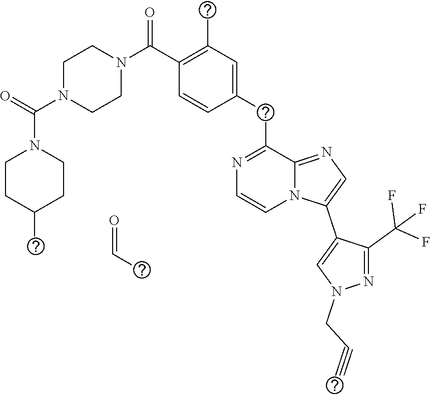 | 2-[4-[8-4-[4-(4- aminopiperidine- 1- carbonyl)piperazine- 1-carbonyl]-3- chloroanilino] imidazo[1,2- a]pyrazin-3-yl]-3- (trifluoromethyl) pyrazol-1- yl]acetonitrile formate | 656.1 | 656.3 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and 4-BOC-aminopiperidine followed by acidic Boc deprotection |
| 179 | 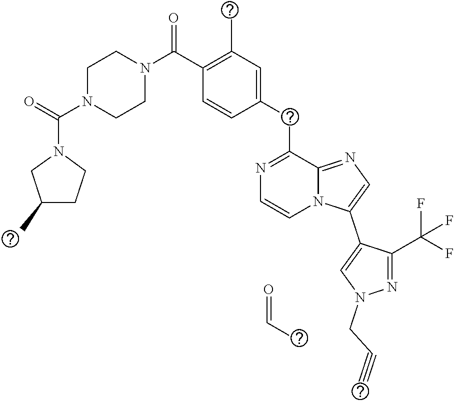 | 2-[4-[8-4-[4- [(3R)-3- aminopyrrolidine- 1- carbonyl]piperazine- 1-carbonyl]-3- chloroanilino] imidazo[1,2- a]pyrazin-3-yl]-3- (trifluoromethyl) pyrazol-1- yl]acetonitrile formate | 642.0 | 642.3 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and (R)-3-(BOC- amino)pyrrolidine followed by acidic Boc deprotection |
| 180 | 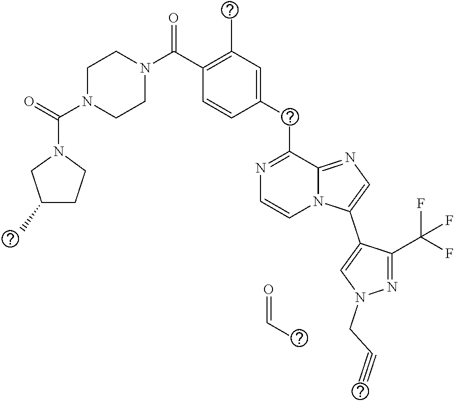 | 2-[4-[8-[4-[4- [(3S)-3- aminopyrrolidine- 1- carbonyl]piperazine- 1-carbonyl]-3- chloroanilino] imidazo[1,2- a]pyrazin-3-yl]-3- (trifluoromethyl) pyrazol-1- yl]acetonitrile formate | 642.0 | 642.3 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and (S)-3-(BOC-amino)pyrrolidine followed by acidic Boc deprotection |
| 181 | 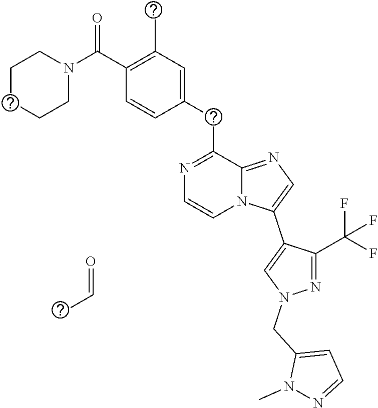 | [2-chloro-4-[[3- [1-[(2- methylpyrazol-3- yl)methyl]-3- (trifluoromethyl) pyrazol-4- yl]imidazo[1,2- a]pyrazin-8- yl]amino]phenyl]- piperazin-1- ylmethanone formate | 585.0 | 585.2 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 5-(chloromethyl)-1-methyl- pyrazole and acidic Boc deprotection |
| 182 | 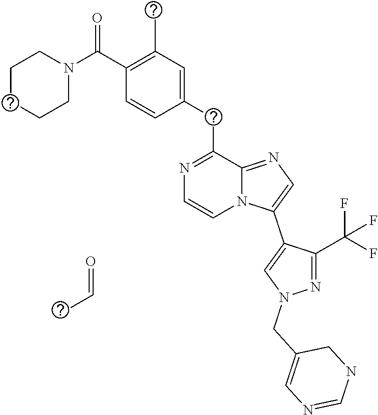 | [2-chloro-4-[[3- [1-(pyrimidin-5- ylmethyl)-3- (trifluoromethyl) pyrazol-4- yl]imidazo[1,2- a]pyrazin-8- yl]amino]phenyl]- piperazin-1- ylmethanone formate | 583.0 | 583.3 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 5-(chloromethyl)pyrimidine hydrochloride and acidic Boc deprotection |
| 183 | 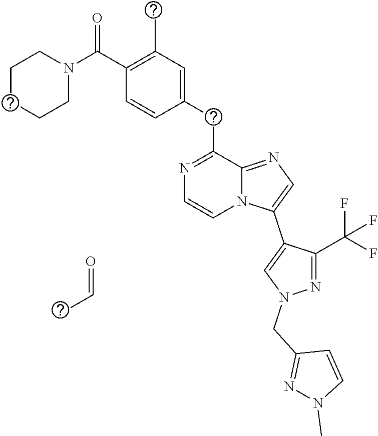 | [2-chloro-4-[[3- [1-[(1- methylpyrazol-3- yl)methyl]-3- (trifluoromethyl) pyrazol-4- yl]imidazo[1,2- a]pyrazin-8- yl]amino]phenyl]- piperazin-1- ylmethanone formate | 585.0 | 585.3 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 3-(chloromethyl)-1-methyl- 1H-pyrazole and acidic Boc deprotection |
| 184 | 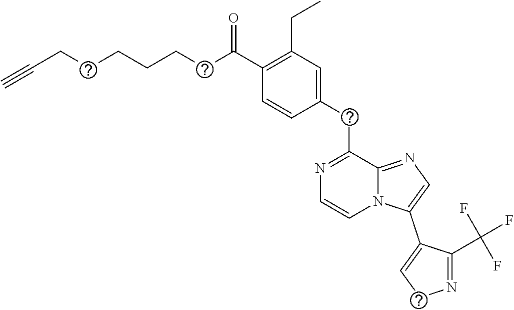 [SMILES: C#CCCCCCCC(=O)c1ccc(Cc2nccn3c(C4=CCN=C4C(F)(F)F)cnc23)cc1CC] | 2-ethyl-N-[3- (prop-2- ynylamino)propyl]- 4-[[3-[3- (trifluoromethyl)- 1H-pyrazol-4- yl]imidazo[1,2- a]pyrazin-8- yl]amino]benzamide | 510.5 | 511.4 | in analogy to the procedures for example 3: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid (example 3, step 2) with tert-butyl (3- aminopropyl)(prop-2-yn-1- yl)carbamate followed by acidic Boc deprotection |
| 185 | 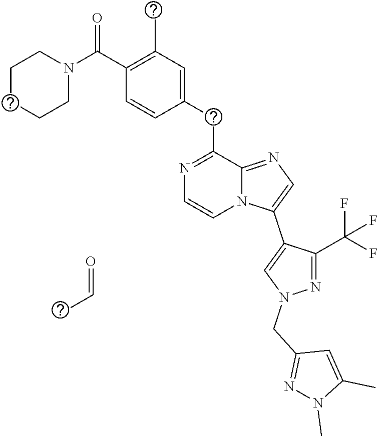 | [2-chloro-4-[[3- [1-[(1,5- dimethylpyrazol- 3-yl)methyl]-3- (trifluoromethyl) pyrazol-4- yl]imidazo[1,2- a]pyrazin-8- yl]amino]phenyl]- piperazin-1- ylmethanone formate | 599.0 | 599.3 | in analogy to the procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 3-(chloromethyl)-1,5- dimethyl-1H-pyrazole and acidic Boc deprotection |
| 186 | 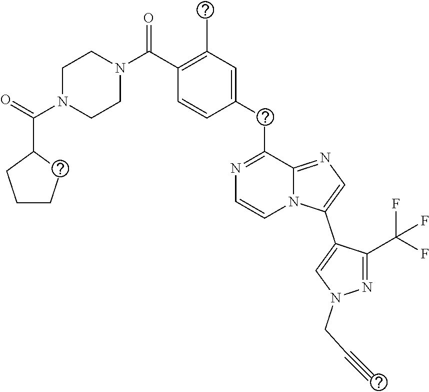 [SMILES: C#CCn1cc(-c2cnc3c(Cc4ccc(C(=O)N5CCN(C(=O)C6CCCC6)CC5)c(C)c4)nccn23)c(C(F)(F)F)n1] | 2-[4-[8-[3- chloro-4-[4-[(2R)- pyrrolidine-2- carbonyl]piperazine- 1-carbonyl]anilino] imidazo[1,2- a]pyrazin-3-yl]-3- (trifluoromethyl) pyrazol-1- yl]acetonitrile | 627.0 | 627.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (tert- butoxycarbonyl)-D-proline, alkylation with 2- bromoacetonitrile and acidic Boc deprotection |
| 187 | 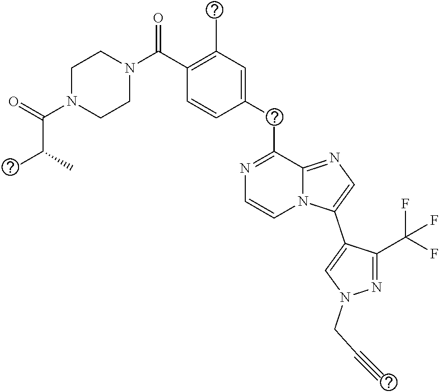 | 2-[4-[8-[4-[4- [(2S)-2- aminopropanoyl] piperazine-1- carbonyl]-3- chloroanilino] imidazo[1,2- a]pyrazin-3-yl]-3- (trifluoromethyl) pyrazol-1- yl]acetonitrile | 601.0 | 601.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (tert- butoxycarbonyl)-L-alanine, alkylation with 2- bromoacetonitrile and acidic Boc deprotection |
| 188 | 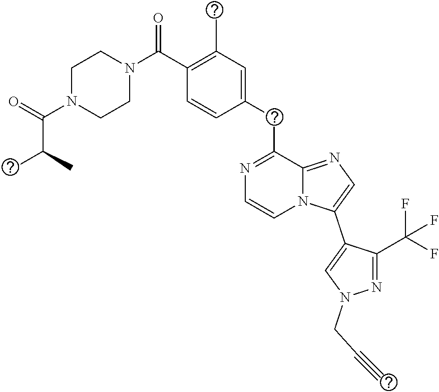 | 2-[4-[8-[4-[4- [(2R)-2- aminopropanoyl] piperazine-1- carbonyl]-3- chloroanilino] imidazo[1,2- a]pyrazin-3-yl]-3- (trifluoromethyl) pyrazol-1- yl]acetonitrile | 601.0 | 601.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (tert- butoxycarbonyl)-D-alanine, alkylation with 2- bromoacetonitrile and acidic Boc deprotection |
| 189 | 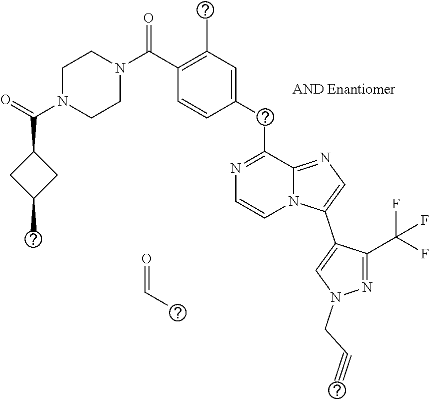 | 2-[4-[8-[4-[4-(3- aminocyclobutane carbonyl)piperazine- 1-carbonyl]-3- chloroanilino] imidazo[1,2- a]pyrazin-3-yl]-3- (trifluoromethyl) pyrazol-1- yl]acetonitrile formate | 627.0 | 627.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 3-(tert- butoxycarbonylamino)cyclobutane carboxylic acid, alkylation with 2- bromoacetonitrile and acidic Boc deprotection |
| 190 | 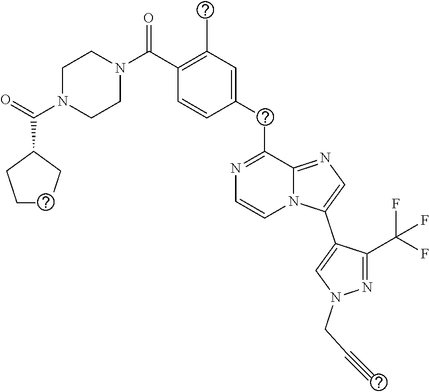 | 2-[4-[8-[3- chloro-4-[4-[(3S)- pyrrolidine-3- carbonyl]piperazine- 1- carbonyl]anilino] imidazo[1,2- a]pyrazin-3-yl]-3- (trifluoromethyl) pyrazol-1- yl]acetonitrile | 627.0 | 627.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (S)-1- (tert-butoxycarbonyl)pyrrolidine- 3-carboxylic acid, alkylation with 2-bromoacetonitrile and acidic Boc deprotection |
| 191 | 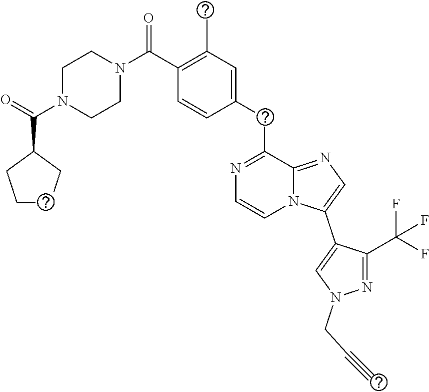 | 2-[4-[8-[3- chloro-4-[4-[(3R)- pyrrolidine-3- carbonyl]piperazine- 1- carbonyl]anilino] imidazo[1,2- a]pyrazin-3-yl]-3- (trifluoromethyl) pyrazol-1- yl]acetonitrile | 627.0 | 627.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (R)-1- (tert-butoxycarbonyl)pyrrolidine- 3-carboxylic acid, alkylation with 2-bromoacetonitrile and acidic Boc deprotection |
| 192 | 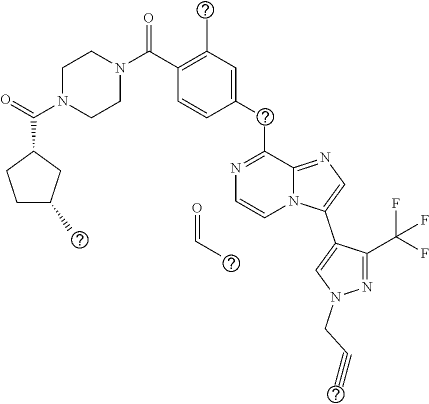 | 2-[4-[8-[4-[4- [(1S,3R)-3- aminocyclopentane carbonyl]piperazine- 1-carbonyl]-3- chloroanilino] imidazo[1,2- a]pyrazin-3-yl]-3- (trifluoromethyl) pyrazol-1- yl]acetonitrile formate | 641.1 | 641.3 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and (1R,3R)-N-BOC-3-amino cyclopentanecarboxylic acid followed by acidic Boc deprotection |
| 193 | 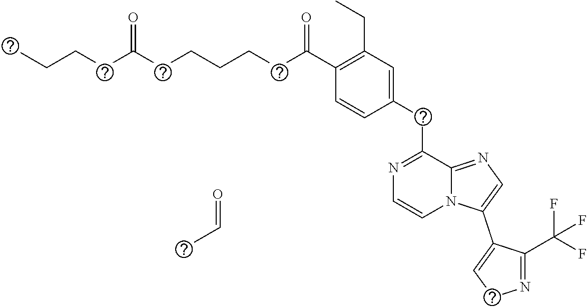 | N-[3-(2- aminoethylcarbamoyl amino)propyl]- 2-ethyl-4-[[3-[3- (trifluoromethyl)- 1H-pyrazol-4- yl]imidazo[1,2- a]pyrazin-8- yl]amino]benzamide formate | 558.6 | 559.2 | in analogy to the procedures for example 6: urea formation of N-(3- aminopropyl)-2-ethyl-4-[[3-[3- (trifluoromethyl)-1H-pyrazol-4- yl]imidazo[1,2-a]pyrazin-8- yl]amino]benzamide (example 13, step 3) with N-BOC- ethylenediamine and acidic Boc deprotection |
| 194 | 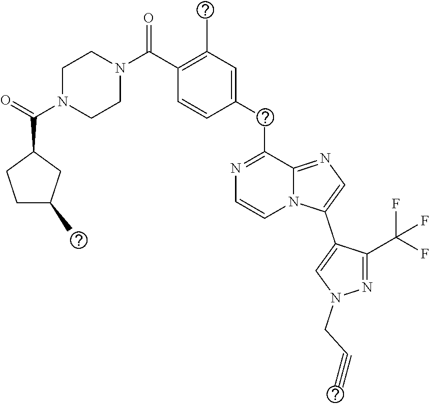 [SMILES: C#CCn1cc(-c2cnc3c(Cc4ccc(C(=O)N5CCN(C(=O)[C@@H]6CC[C@H](C)C6)CC5)c(C)c4)nccn23)c(C(F)(F)F)n1] | 2-[4-[8-[4-[4- [(1R,3S)-3- aminocyclopentane carbonyl]piperazine- 1-carbonyl]-3- chloroanilino] imidazo[1,2- a]pyrazin-3-yl]-3- (trifluoromethyl) pyrazol-1- yl]acetonitrile formate | 641.1 | 641.3 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and (−)-(1R,3S)-N-BOC-3- aminocyclopentanecarboxylic acid followed by acidic Boc deprotection |
| 195 | 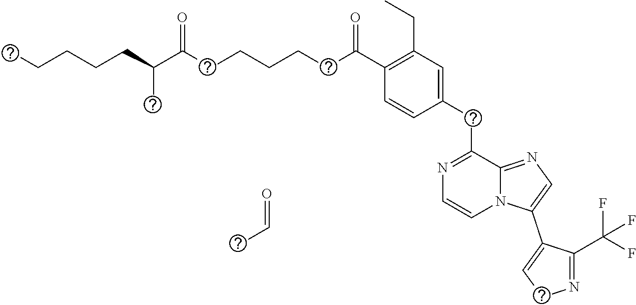 | N-[3-[[(2S)-2,6- diaminohexanoyl] amino]propyl]-2- ethyl-4-[[3-[3- (trifluoromethyl)- 1H-pyrazol-4- yl]imidazo[1,2- a]pyrazin-8- yl]amino]benzamide formate | 600.6 | 601.4 | in analogy to the procedures for example 6: amide coupling of N- (3-aminopropyl)-2-ethyl-4-[[3-[3- (trifluoromethyl)-1H-pyrazol-4- yl]imidazo[1,2-a]pyrazin-8- yl]amino]benzamide (example 13, step 3) with BOC-LYS(BOC)-OH and acidic Boc deprotection |
| 196 | 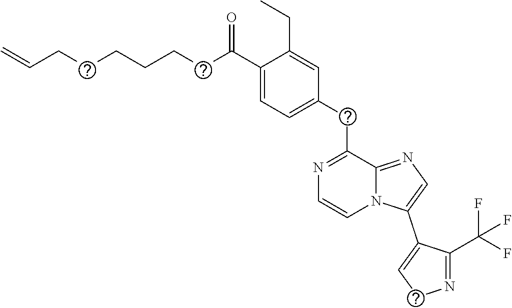 [SMILES: C=CCCCCCCC(=O)c1ccc(Cc2nccn3c(C4=CCN=C4C(F)(F)F)cnc23)cc1CC] | 2-ethyl-N-[3- (prop-2- enylamino)propyl]- 4-[[3-[3- (trifluoromethyl)- 1H-pyrazol-4- yl]imidazo[1,2- a]pyrazin-8- yl]amino]benzamide | 512.5 | 513.4 | in analogy to the procedures for example 3: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid (example 3, step 2) with tert-butyl allyl(3- aminopropyl)carbamate followed by acidic Boc deprotection |
| 197 | 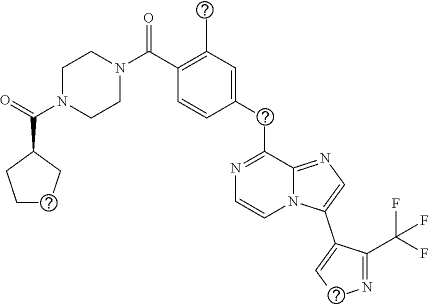 | [2-chloro-4-[[3- [3- (trifluoromethyl)- 1H-pyrazol-4- yl]imidazo[1,2- a]pyrazin-8- yl]amino]phenyl]- [4-[(3R)- pyrrolidine-3- carbonyl]piperazin- 1-yl]methanone | 588.0 | 588.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (R)-1- (tert-butoxycarbonyl)pyrrolidine- 3-carboxylic acid and acidic Boc deprotection |
| 198 | 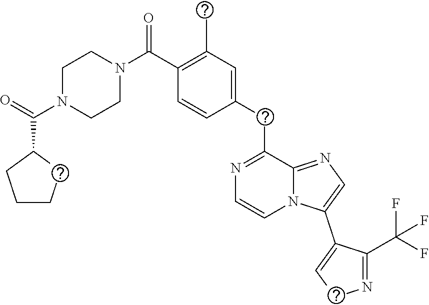 | [2-chloro-4-[[3- [3- (trifluoromethyl)- 1H-pyrazol-4- yl]imidazo[1,2- a]pyrazin-8- yl]amino]phenyl]- [4-[(2R)- pyrrolidine-2- carbonyl]piperazin- 1-yl]methanone | 588.0 | 588.4 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (R)-1- (tert-butoxycarbonyl)pyrrolidine- 2-carboxylic acid and acidic Boc deprotection |
| 199 | 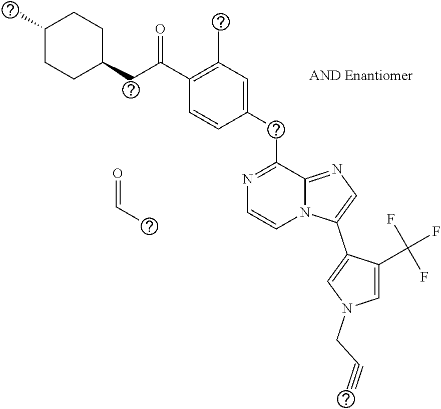 | N-(4- aminocyclohexyl)- 2-chloro-4-[[3- [1-(cyanomethyl)- 3- (trifluoromethyl) pyrazol-4- yl]imidazo[1,2- a]pyrazin-8- yl]amino]benzamide formate | 558.0 | 558.2 | in analogy to the procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with trans-N-Boc-1,4- cyclohexandiamine and acidic Boc deprotection |
| 200 | 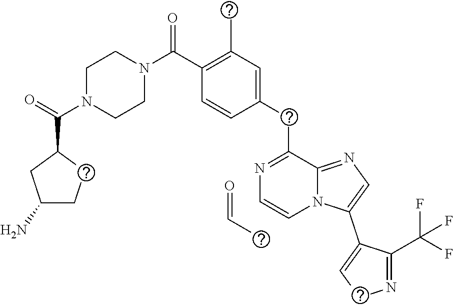 | [4-[(2S,4R)-4- aminopyrrolidine- 2- carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 603.0 | 603.1 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (2S,4R)- 1-tert-butoxycarbonyl-4-(tert- butoxycarbonylamino)pyrrolidine- 2-carboxylic acid and acidic Boc deprotection |
| 201 | 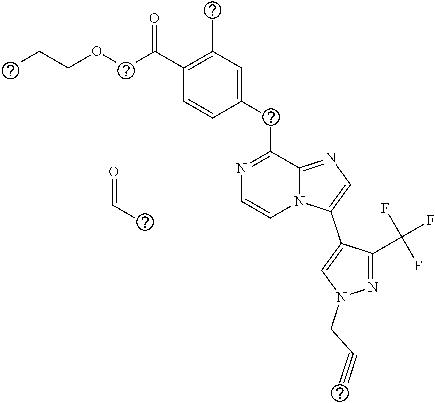 | N-(2- aminoethoxy)-2- chloro-4-[[3-[1- (cyanomethyl)-3- (trifluoromethyl) pyrazol-4- yl]imidazo[1,2- a]pyrazin-8- yl]amino]benzamide formate | 519.9 | 520.1 | in analogy to the procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with tert-butyl N-(2- aminooxyethyl)carbamate and acidic Boc deprotection |
| 202 | 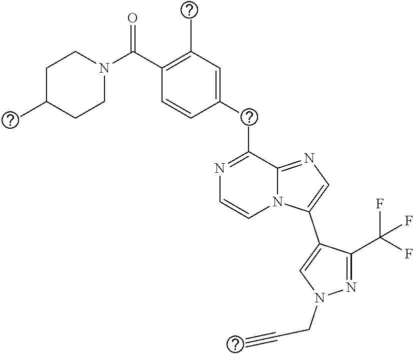 [SMILES: C#CCn1cc(-c2cnc3c(Cc4ccc(C(=O)N5CCC(C)CC5)c(C)c4)nccn23)c(C(F)(F)F)n1] | 2-[4-[8-[4-(4- aminopiperidine- 1-carbonyl)-3- chloroanilino] imidazo[1,2- a]pyrazin-3-yl]-3- (trifluoromethyl) pyrazol-1- yl]acetonitrile | 543.9 | 544.3 | in analogy to the procedures for example 11: alkylation of tert- butyl N-[1-[2-chloro-4-[[3-[3- (trifluoromethyl)-1H-pyrazol-4- yl]imidazo[1,2-a]pyrazin-8- yl]amino]benzoyl]-4- piperidyl]carbamate (example 11, step 1) with 2-bromoacetonitrile [CAS#590-17-0] and subsequent acidic Boc deprotection |
| 203 | 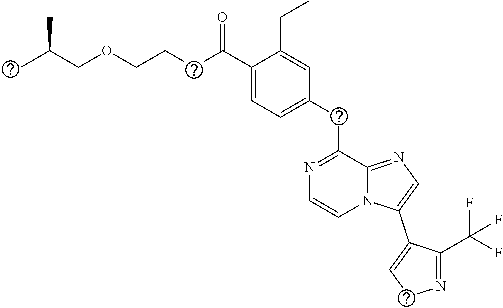 | N-[2-[(2S)-2- aminopropoxy] ethyl]-2-ethyl-4-[[3- [3- (trifluoromethyl)- 1H-pyrazol-4- yl]imidazo[1,2- a]pyrazin-8- yl]amino]benzamide | 516.5 | 517.4 | in analogy to the procedures for example 1: amide coupling of 2- ethyl-4-[(3-iodoimidazo[1,2- a]pyrazin-8-yl)amino]benzoic acid, hydrochloride with tert-butyl (S)- (1-(2-aminoethoxy)propan-2- yl)carbamate, subsequent Suzuki reaction with (3-(trifluoromethyl)- 1H-pyrazol-4-yl)boronic acid and acidic Boc deprotection |
| 204 | 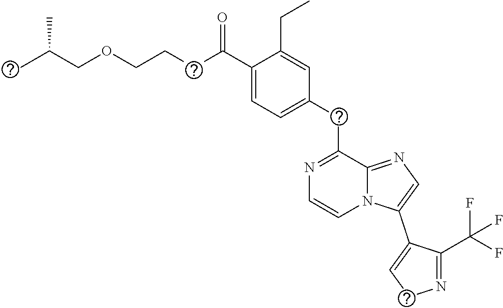 | N-[2-[(2R)-2- aminopropoxy] ethyl]-2-ethyl-4-[[3- [3- (trifluoromethyl)- 1H-pyrazol-4- yl]imidazo[1,2- a]pyrazin-8- yl]amino]benzamide | 516.5 | 517.4 | in analogy to the procedures for example 1: amide coupling of 2- ethyl-4-[(3-iodoimidazo[1,2- a]pyrazin-8-yl)amino]benzoic acid, hydrochloride with tert-butyl (R)- (1-(2-aminoethoxy)propan-2- yl)carbamate, subsequent Suzuki reaction with (3-(trifluoromethyl)- 1H-pyrazol-4-yl)boronic acid and acidic Boc deprotection |
| 205 | 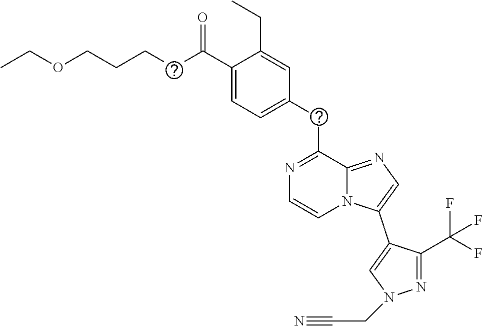 [SMILES: CCOCCCCC(=O)c1ccc(Cc2nccn3c(-c4cn(CC#N)nc4C(F)(F)F)cnc23)cc1CC] | 4-[[3-[1- (cyanomethyl)-3- (trifluoromethyl) pyrazol-4- yl]imidazo[1,2- a]pyrazin-8- yl]amino]-N-(3- ethoxypropyl)-2- ethylbenzamide | 540.5 | 541.4 | in analogy to the procedures for example 3: amide formation with 2-ethyl-4-[[3-[3-(trifluoromethyl)- 1H-pyrazol-4-yl]imidazo[1,2- a]pyrazin-8-yl]amino]benzoic acid, hydrochloric acid (example 3, step 2) with N-ethyl-N- isopropylpropan-2-amine; alkylation with 2- bromoacetonitrile [CAS#590-17-0] and subsequent acidic Boc deprotection |
| 206 | 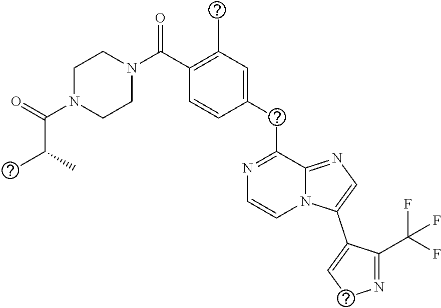 | (2S)-2-amino-1- [4-[2-chloro-4- [[3-[3- (trifluoromethyl)- 1H-pyrazol-4- yl]imidazo[1,2- a]pyrazin-8- yl]amino]benzoyl] piperazin-1- yl]propan-1-one | 562.0 | 562.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (tert- butoxycarbonyl)-L-alanine and acidic Boc deprotection |
| 207 | 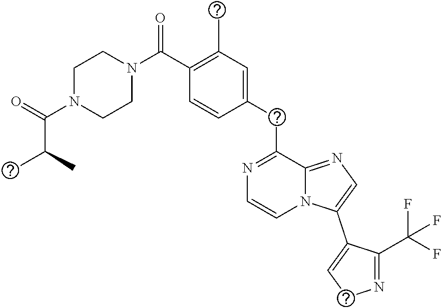 | (2R)-2-amino-1- [4-[2-chloro-4- [[3-[3- (trifluoromethyl)- 1H-pyrazol-4- yl]imidazo[1,2- a]pyrazin-8- yl]amino]benzoyl] piperazin-1- yl]propan-1-one | 562.0 | 562.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (tert- butoxycarbonyl)-D-alanine and acidic Boc deprotection |
| 208 | 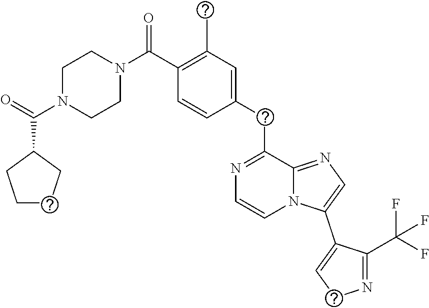 | [2-chloro-4-[[3- [3- (trifluoromethyl)- 1H-pyrazol-4- yl]imidazo[1,2- a]pyrazin-8- yl]amino]phenyl]- [4-[(3S)- pyrrolidine-3- carbonyl]piperazin- 1-yl]methanone | 588.0 | 588.4 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (S)-1- (tert-butoxycarbonyl)pyrrolidine- 3-carboxylic acid and acidic Boc deprotection |
| 209 | 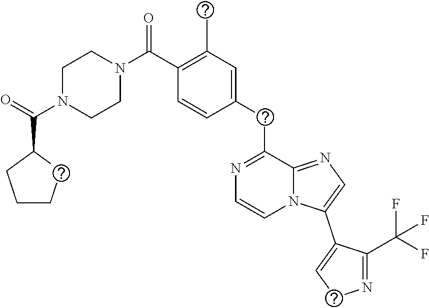 | [2-chloro-4-[[3- [3- (trifluoromethyl)- 1H-pyrazol-4- yl]imidazo[1,2- a]pyrazin-8- yl]amino]phenyl]- [4-[(2S)- pyrrolidine-2- carbonyl]piperazin- 1-yl]methanone | 588.0 | 588.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (S)-1- (tert-butoxycarbonyl)pyrrolidine- 2-carboxylic acid and acidic Boc deprotection |
| 210 | 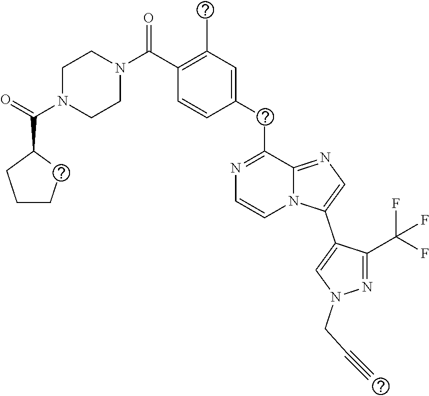 | 2-[4-[8-[3- chloro-4-[4-[(2S)- pyrrolidine-2- carbonyl]piperazine- 1- carbonyl]anilino] imidazo[1,2- a]pyrazin-3-yl]-3- (trifluoromethyl) pyrazol-1- yl]acetonitrile | 627.0 | 627.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (S)-1- (tert-butoxycarbonyl)pyrrolidine- 2-carboxylic acid, alkylation with 2-bromoacetonitrile [CAS#590-17- 0] and acidic Boc deprotection |
| 211 | 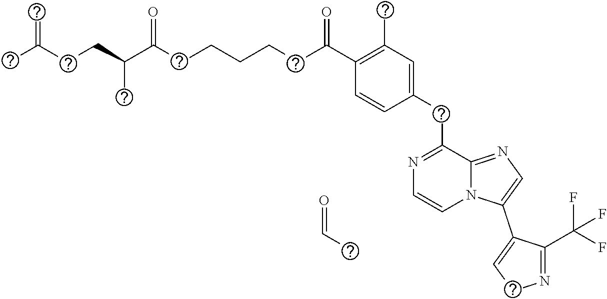 | N-[3-[[(2S)-2- amino-3- carbamimidamido propanoyl]amino] propyl]-2-chloro- 4-[[3-[3- (trifluoromethyl)- 1H-pyrazol-4- yl]imidazo[1,2- a]pyrazin-8- yl]amino]benzamide formate | 607.0 | 607.1 | in analogy to the procedures for example 1: amide coupling of 2- chloro-4-[(3-iodoimidazo[1,2- a]pyrazin-8-yl)amino]benzoic acid (example 14, step 1) with N-BOC- 1,3-diaminopropane, subsequent Suzuki reaction with (3- (trifluoromethyl)-1H-pyrazol-4- yl)boronic acid, acidic Boc deprotection, coupling with (2S)- 2-(tert-butoxycarbonylamino)-3- guanidino-propanoic acid and subsequent acidic Boc deprotection |
| 212 | 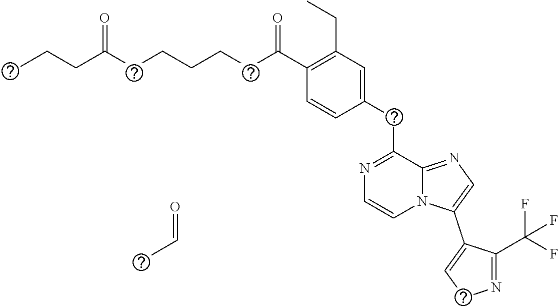 | N-[3-(3- aminopropanoyl amino)propyl]-2- ethyl-4-[[3-[3- (trifluoromethyl)- 1H-pyrazol-4- yl]imidazo[1,2- a]pyrazin-8- yl]amino]benzamide formate | 543.6 | 544.3 | in analogy to the procedures for example 6: amide coupling of N- (3-aminopropyl)-2-ethyl-4-[[3-[3- (trifluoromethyl)-1H-pyrazol-4- yl]imidazo[1,2-a]pyrazin-8- yl]amino]benzamide (example 13, step 3) with BOC-BETA-ALA-OH and acidic Boc deprotection |
| 213 | 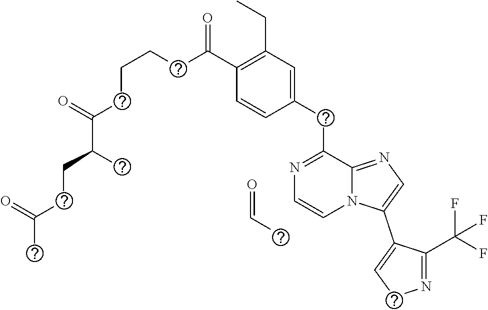 | N-[2-[[(2S)-2- amino-3- carbamimidamido propanoyl]amino] ethyl]-2-ethyl-4- [[3-[3- (trifluoromethyl)- 1H-pyrazol-4- yl]imidazo[1,2- a]pyrazin-8- yl]amino]benzamide formate | 586.6 | 587.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-BOC-ethylenediamine, subsequent Suzuki reaction with (3-(trifluoromethyl)-1H-pyrazol-4- yl)boronic acid, acidic Boc deprotection, coupling with (2S)- 2-(tert-butoxycarbonylamino)-3- guanidino-propanoic acid and subsequent acidic Boc deprotection |
| 214 | 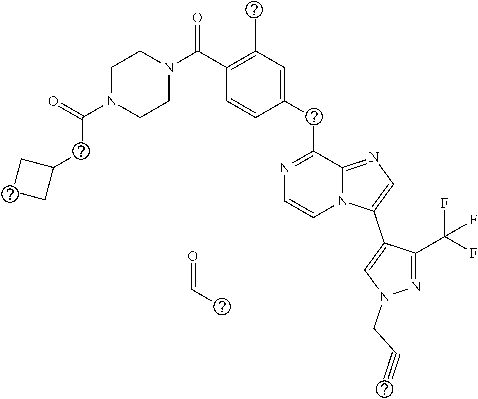 | N-(azetidin-3-yl)- 4-[2-chloro-4-[[3- [1-(cyanomethyl)- 3- (trifluoromethyl) pyrazol-4- yl]imidazo[1,2- a]pyrazin-8- yl]amino]benzoyl] piperazine-1- carboxamide formate | 628.0 | 628.1 | in analogy to the procedures for example 12: urea formation of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) with 1-BOC-3-(amino)azetidine followed by acidic Boc deprotection |
| 215 | 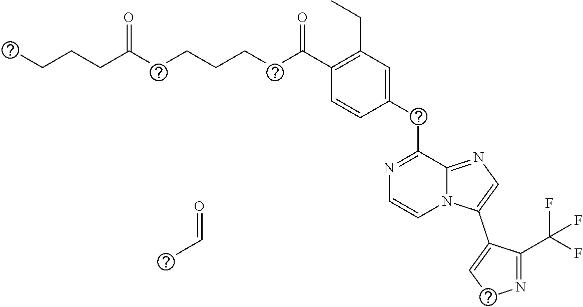 | N-[3-(4- aminobutanoyl amino)propyl]-2- ethyl-4-[[3-[3- (trifluoromethyl)- 1H-pyrazol-4- yl]imidazo[1,2- a]pyrazin-8- yl]amino]benzamide formate | 557.6 | 558.3 | in analogy to the procedures for example 6: amide coupling of N- (3-aminopropyl)-2-ethyl-4-[[3-[3- (trifluoromethyl)-1H-pyrazol-4- yl]imidazo[1,2-a]pyrazin-8- yl]amino]benzamide (example 13, step 3) with BOC-gamma-abu-OH and acidic Boc deprotection |
| 216 | 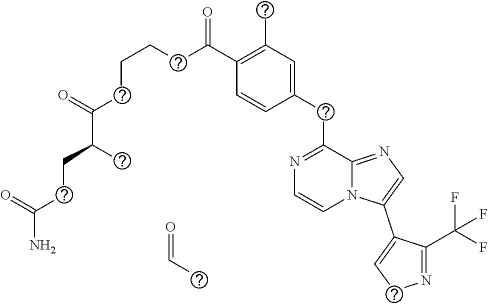 | N-[2-[[(2S)-2- amino-3- carbamimidamido propanoyl]amino] ethyl]-2-chloro-4- [[3-[3- (trifluoromethyl)- 1H-pyrazol-4- yl]imidazo[1,2- a]pyrazin-8- yl]amino]benzamide formate | 593.0 | 593 | in analogy to the procedures for example 1: amide coupling of 2- chloro-4-[(3-iodoimidazo[1,2- a]pyrazin-8-yl)amino]benzoic acid (example 14, step 1) with N-BOC- ethylenediamine, subsequent Suzuki reaction with (3- (trifluoromethyl)-1H-pyrazol-4- yl)boronic acid, acidic Boc deprotection, coupling with (2S)- 2-(tert-butoxycarbonylamino)-3- guanidino-propanoic acid and subsequent acidic Boc deprotection |
| 217 | 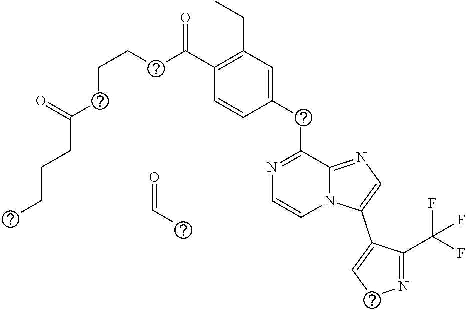 | N-[2-(4- aminobutanoyl amino)ethyl]-2-ethyl- 4-[[3-[3- (trifluoromethyl)- 1H-pyrazol-4- yl]imidazo[1,2- a]pyrazin-8- yl]amino]benzamide formate | 543.6 | 544.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-BOC-ethylenediamine, subsequent Suzuki reaction with (3-(trifluoromethyl)-1H-pyrazol-4- yl)boronic acid, acidic Boc deprotection, coupling with BOC- gamma-abu-OH and subsequent acidic Boc deprotection |
| 218 | 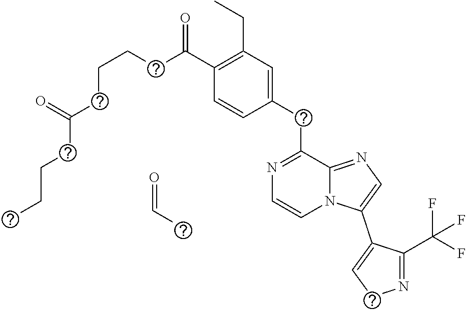 | N-[2-(2- aminoethyl carbamoylamino)ethyl]- 2-ethyl-4-[[3-[3- (trifluoromethyl)- 1H-pyrazol-4- yl]imidazo[1,2- a]pyrazin-8- yl]amino]benzamide formate | 544.5 | 545.1 | in analogy to the procedures for example 12: urea formation of N- (2-aminoethyl)-2-ethyl-4-[[3-[3- (trifluoromethyl)-1H-pyrazol-4- yl]imidazo[1,2-a]pyrazin-8- yl]amino]benzamide (example 3, step 4) with N-BOC- ethylenediamine followed by acidic Boc deprotection |
| 219 | 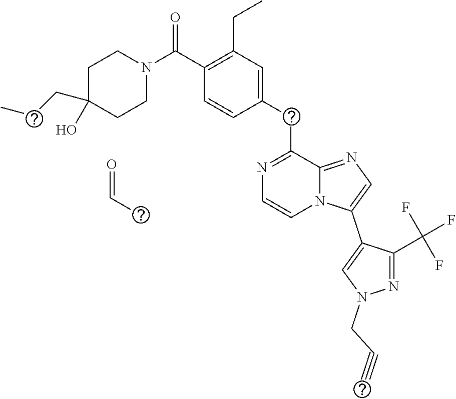 | 2-[4-[8-[3-ethyl- 4-[4-hydroxy-4- (methylamino methyl)piperidine-1- carbonyl]anilino] imidazo[1,2- a]pyrazin-3-yl]-3- (trifluoromethyl) pyrazol-1- yl]acetonitrile formate | 581.6 | 582.3 | in analogy to the procedures for example 1: Suzuki reaction of 2- ethyl-4-[(3-iodoimidazo[1,2- a]pyrazin-8-yl)amino]benzoic acid, hydrochloride (example 1, step 2) with 2-[4-(4,4,5,5-tetramethyl- 1,3,2-dioxaborolan-2-yl)-3- (trifluoromethyl)pyrazol-1- yl]acetonitrile, subsequent amide formation with tert-butyl N-[(4- hydroxy-4-piperidyl)methyl]-N- methyl-carbamate and subsequent Boc deprotection with trimethylsilyl trifluormethanesulfonate |
| 220 | 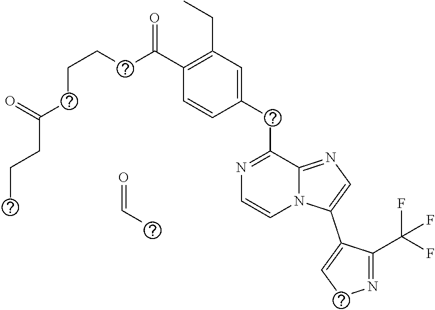 | N-[2-(3- aminopropanoyl amino)ethyl]-2- ethyl-4-[[3-[3- (trifluoromethyl)- 1H-pyrazol-4- yl]imidazo[1,2- a]pyrazin-8- yl]amino]benzamide formate | 529.5 | 530.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-BOC-ethylenediamine, subsequent Suzuki reaction with (3-(trifluoromethyl)-1H-pyrazol-4- yl)boronic acid, acidic Boc deprotection, coupling with BOC- BETA-ALA-OH and subsequent acidic Boc deprotection |
| 221 | 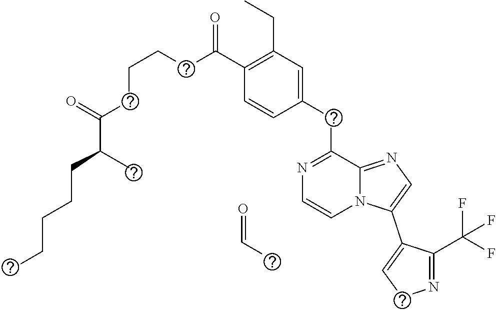 | N-[2-[[(2S)-2,6- diaminohexanoyl] amino]ethyl]-2- ethyl-4-[[3-[3- (trifluoromethyl)- 1H-pyrazol-4- yl]imidazo[1,2- a]pyrazin-8- yl]amino]benzamide formate | 586.6 | 587.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-BOC-ethylenediamine, subsequent Suzuki reaction with (3-(trifluoromethyl)-1H-pyrazol-4- yl)boronic acid, acidic Boc deprotection, coupling with BOC- gamma-abu-OH and subsequent acidic Boc deprotection |
| 222 | 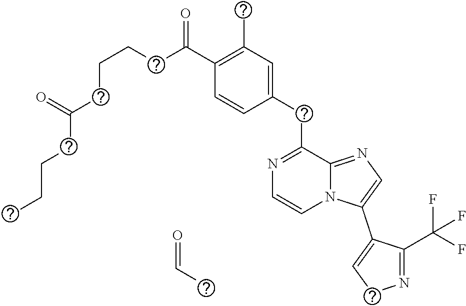 | N-[2-(2- aminoethylcarba moylamino)ethyl]- 2-chloro-4-[[3-[3- (trifluoromethyl)- 1H-pyrazol-4- yl]imidazo[1,2- a]pyrazin-8- yl]amino]benzamide formate | 550.9 | 551 | in analogy to the procedures for example 1: amide coupling of 2- chloro-4-[(3-iodoimidazo[1,2- a]pyrazin-8-yl)amino]benzoic acid (example 14, step 1) with N-BOC- ethylenediamine, subsequent Suzuki reaction with (3- (trifluoromethyl)-1H-pyrazol-4- yl)boronic acid, acidic Boc deprotection, coupling with N- BOC-ethylenediamine and subsequent acidic Boc deprotection |
| 223 | 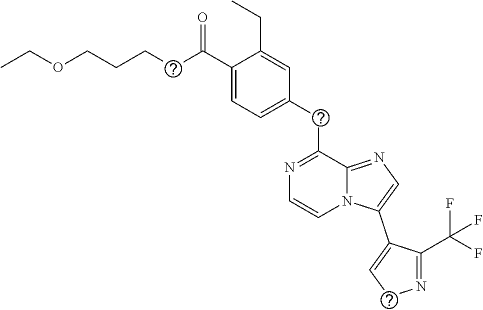 [SMILES: CCOCCCCC(=O)c1ccc(Cc2nccn3c(C4=CCN=C4C(F)(F)F)cnc23)cc1CC] | N-(3- ethoxypropyl)-2- ethyl-4-[[3-[3- (trifluoromethyl)- 1H-pyrazol-4- yl]imidazo[1,2- a]pyrazin-8- yl]amino]benzamide | 501.5 | 502.4 | in analogy to the procedures for example 1: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid (example 3, ste 2) with N-ethyl-N- isopropylpropan-2-amine |
| 224 | 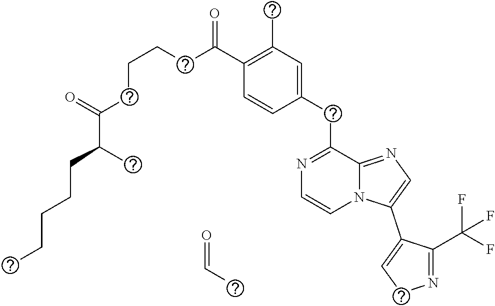 | 2-chloro-N-[2- [[(2S)-2,6- diaminohexanoyl] amino]ethyl]-4- [[3-[3- (trifluoromethyl)- 1H-pyrazol-4- yl]imidazo[1,2- a]pyrazin-8- yl]amino]benzamide formate | 593.0 | 593.1 | in analogy to the procedures for example 1: amide coupling of 2- chloro-4-[(3-iodoimidazo[1,2- a]pyrazin-8-yl)amino]benzoic acid (example 14, step 1) with N-BOC- ethylenediamine, subsequent Suzuki reaction with (3- (trifluoromethyl)-1H-pyrazol-4- yl)boronic acid, acidic Boc deprotection, coupling with BOC- LYS(BOC)-OH and subsequent acidic Boc deprotection |
| 225 | 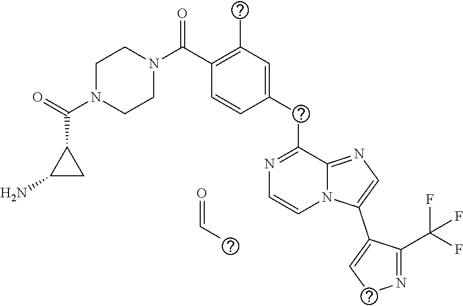 | [4-[(1R,2S)-2- aminocyclopropane carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 574.0 | 575.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (1R,2S)- 2-(tert- butoxycarbonylamino)cyclopropane carboxylic acid and acidic Boc deprotection |
| 226 | 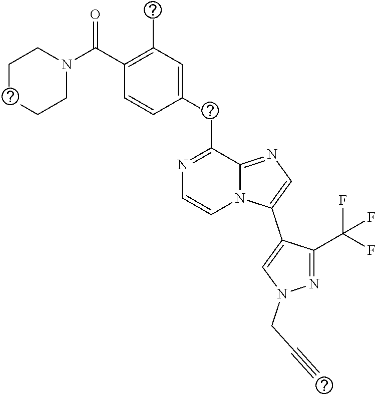 [SMILES: C#CCn1cc(-c2cnc3c(Cc4ccc(C(=O)N5CCCCC5)c(C)c4)nccn23)c(C(F)(F)F)n1] | 2-[4-[8-[3- chloro-4- (piperazine-1- carbonyl)anilino] imidazo[1,2- a]pyrazin-3-yl]-3- (trifluoromethyl) pyrazol-1- yl]acetonitrile | 529.9 | 528.5 [M − H]− | in analogy to procedures for example 6: alkylation of tert-butyl 4-[2-chloro-4-[[3-[3- (trifluoromethyl)-1H-pyrazol-4- yl]imidazo[1,2-a]pyrazin-8- yl]amino]benzoyl]piperazine-1- carboxylate (example 6, step 4) with 2-bromo acetonitrile and acidic Boc deprotection |
| 227 | 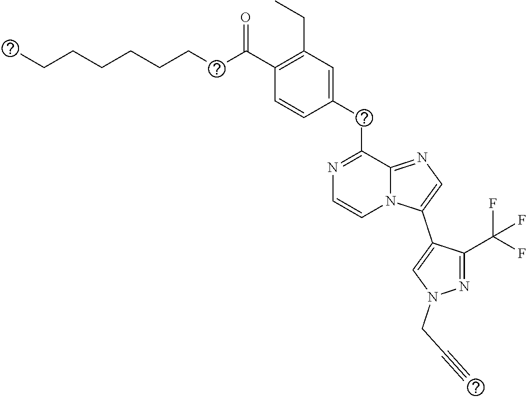 [SMILES: C#CCn1cc(-c2cnc3c(Cc4ccc(C(=O)CCCCCCCC)c(CC)c4)nccn23)c(C(F)(F)F)n1] | N-(6- aminohexyl)-4- [[3-[1- (cyanomethyl)-3- (trifluoromethyl) pyrazol-4- yl]imidazo[1,2- a]pyrazin-8- yl]amino]-2- ethylbenzamide | 553.6 | 554.4 | in analogy to procedures for example 6: amide formation with 2-ethyl-4-[[3-[3-(trifluoromethyl)- 1H-pyrazol-4-yl]imidazo[1,2- a]pyrazin-8-yl]amino]benzoic acid, hydrochloric acid (example 3, step 2) and tert-butyl (6- aminohexyl)carbamate and subsequent acidic Boc deprotection |
| 228 | 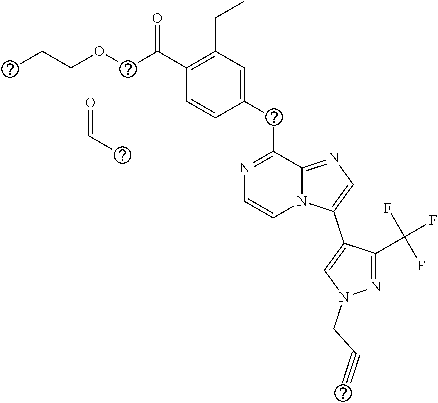 | N-(2- aminoethoxy)-4- [[3-[1- (cyanomethyl)-3- (trifluoromethyl) pyrazol-4- yl]imidazo[1,2- a]pyrazin-8- yl]amino]-2- ethylbenzamide formate | 513.5 | 514.2 | in analogy to the procedures for example 1: Suzuki reaction of 2- ethyl-4-[(3-iodoimidazo[1,2- a]pyrazin-8-yl)amino]benzoic acid, hydrochloride (example 1, step 2) with 2-[4-(4,4,5,5-tetramethyl- 1,3,2-dioxaborolan-2-yl)-3- (trifluoromethyl)pyrazol-1- yl]acetonitrile, subsequent amide formation with tert-butyl N-[(4- hydroxy-4-piperidyl)methyl]-N- methyl-carbamate and subsequent acidic Boc deprotection |
| 229 | 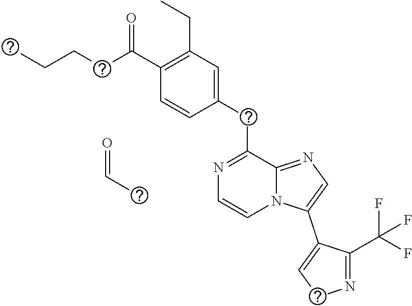 | N-(2- aminoethyl)-2- ethyl-4-[[3-[3- (trifluoromethyl)- 1H-pyrazol-4- yl]imidazo[1,2- a]pyrazin-8- yl]amino]benzamide formate | 458.4 | 459.2 | in analogy to the procedures for example 1: amide coupling of 2- ethyl-4-[(3-iodoimidazo[1,2- a]pyrazin-8-yl)amino]benzoic acid, hydrochloride (example 1, step 2) with N-BOC-ethylenediamine and subsequent acidic Boc deprotection |
| 230 | 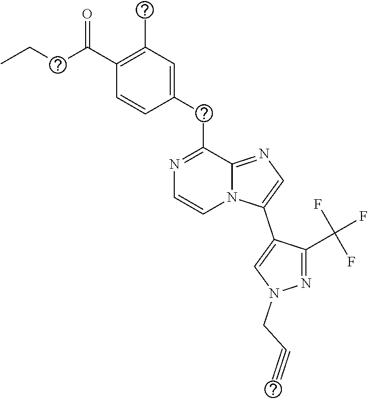 [SMILES: C#CCn1cc(-c2cnc3c(Cc4ccc(C(=O)CCC)c(C)c4)nccn23)c(C(F)(F)F)n1] | 2-chloro-4-[[3- [1-(cyanomethyl)- 3- (trifluoromethyl) pyrazol-4- yl]imidazo[1,2- a]pyrazin-8- yl]amino]-N- ethylbenzamide | 488.9 | 489.1 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with ethylamine hydrochloride |
| 231 | 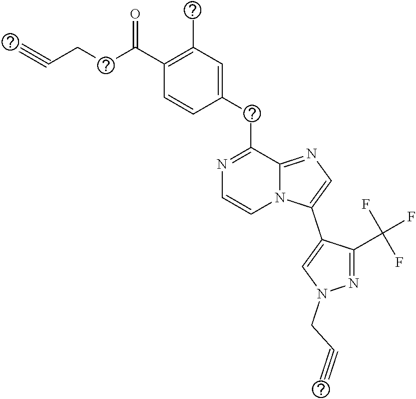 [SMILES: C#CCCC(=O)c1ccc(Cc2nccn3c(-c4cn(CC#C)nc4C(F)(F)F)cnc23)cc1C] | 2-chloro-N- (cyanomethyl)-4- [[3-[1- (cyanomethyl)-3- (trifluoromethyl) pyrazol-4- yl]imidazo[1,2- a]pyrazin-8- yl]amino]benzamide | 499.8 | 500 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with aminoacetonitrile |
| 232 | 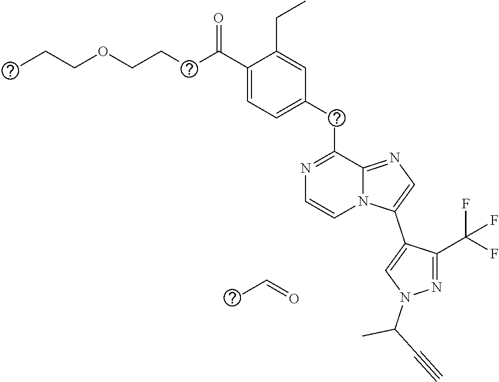 | N-[2-(2- aminoethoxy)ethyl]- 4-[[3-[1-but-3- yn-2-yl-3- (trifluoromethyl) pyrazol-4- yl]imidazo[1,2- a]pyrazin-8- yl]amino]-2- ethylbenzamide formate | 554.6 | 555.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 2-bromopropanenitrile and subsequent acidic cleavage of the Boc group |
| 233 | 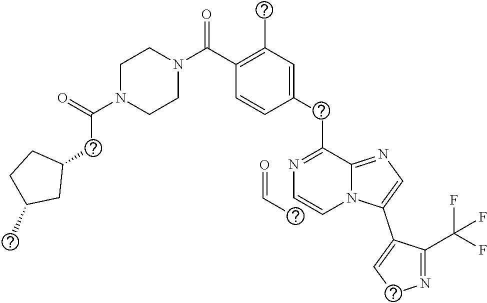 | N-[(1S,3R)-3- aminocyclopentyl]- 4-[2-chloro-4- [[3-[3- (trifluoromethyl)- 1H-pyrazol-4- yl]imidazo[1,2- a]pyrazin-8- yl]amino]benzoyl] piperazine-1- carboxamide formate | 617.0 | 617.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl N-[(1R,3S)-3- aminocyclopentyl]carbamate followed by acidic Boc deprotection |
| 234 | 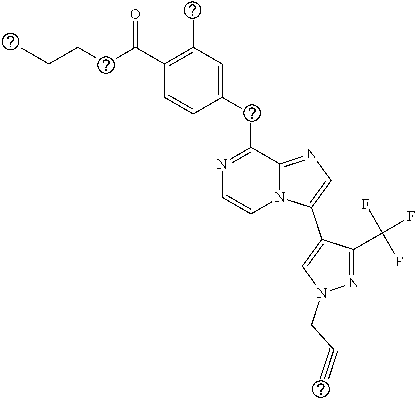 [SMILES: C#CCn1cc(-c2cnc3c(Cc4ccc(C(=O)CCCC)c(C)c4)nccn23)c(C(F)(F)F)n1] | 2-chloro-4-[[3- [1-(cyanomethyl)- 3- (trifluoromethyl) pyrazol-4- yl]imidazo[1,2- a]pyrazin-8- yl]amino]-N-(2- hydroxyethyl) benzamide | 504.9 | 505 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with 2- hydroxyethylamine |
| 235 | 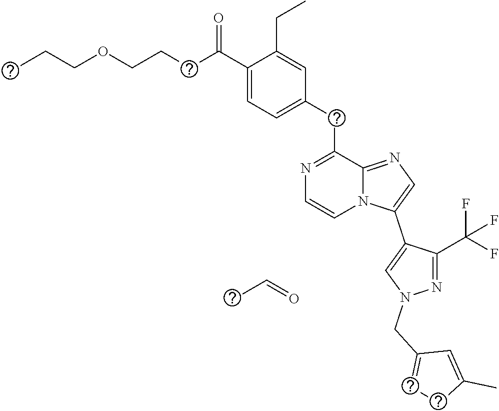 | N-[2-(2- aminoethoxy) ethyl]-2-ethyl-4-[[3- [1-[(5-methyl-1,2- oxazol-3- yl)methyl]-3- (trifluoromethyl) pyrazol-4- yl]imidazo[1,2- a]pyrazin-8- yl]amino]benzamide formate | 597.6 | 598.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 5-(bromomethyl)-3- methylisoxazole and subsequent acidic cleavage of the Boc group |
| 236 | 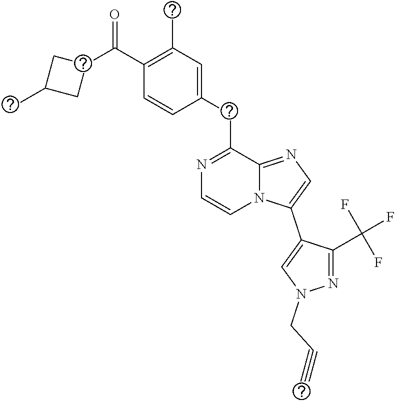 [SMILES: C#CCn1cc(-c2cnc3c(Cc4ccc(C(=O)C5CC(C)C5)c(C)c4)nccn23)c(C(F)(F)F)n1] | 2-[4-[8-[3- chloro-4-(3- hydroxyazetidine- 1- carbonyl)anilino] imidazo[1,2- a]pyrazin-3-yl]-3- (trifluoromethyl) pyrazol-1- yl]acetonitrile | 516.9 | 517.1 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with azetidin-3-ol hydrochloride |
| 237 | 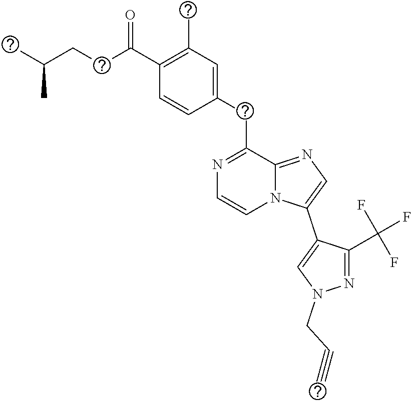 | 2-chloro-4-[[3- [1-(cyanomethyl)- 3- (trifluoromethyl) pyrazol-4- yl]imidazo[1,2- a]pyrazin-8- yl]amino]-N- [(2R)-2- hydroxypropyl] benzamide | 518.9 | 519 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with (R)-(−)-1-amino-2- propanol |
| 238 | 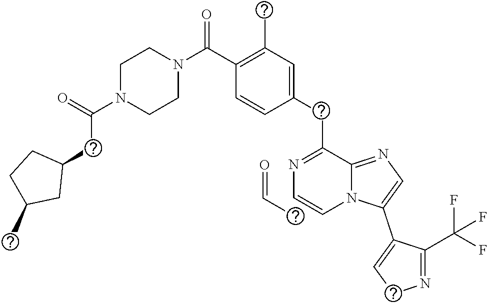 | N-[(1R,3S)-3- aminocyclopentyl]- 4-[2-chloro-4- [[3-[3- (trifluoromethyl)- 1H-pyrazol-4- yl]imidazo[1,2- a]pyrazin-8- yl]amino]benzoyl] piperazine-1- carboxamide formate | 617.0 | 617.1 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyltert-butyl rac-[(1S,3R)-3- aminocyclopentyl]carbamate followed by acidic Boc deprotection |
| 239 | 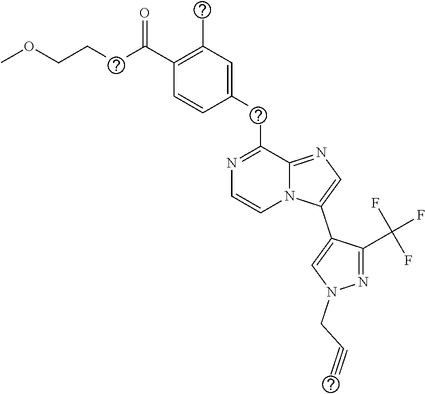 [SMILES: C#CCn1cc(-c2cnc3c(Cc4ccc(C(=O)CCCOC)c(C)c4)nccn23)c(C(F)(F)F)n1] | 2-chloro-4-[[3- [1-(cyanomethyl)- 3- (trifluoromethyl) pyrazol-4- yl]imidazo[1,2- a]pyrazin-8- yl]amino]-N-(2- methoxyethyl) benzamide | 518.9 | 519 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with (R)-(−)-2- methoxyethylamine |
| 240 | 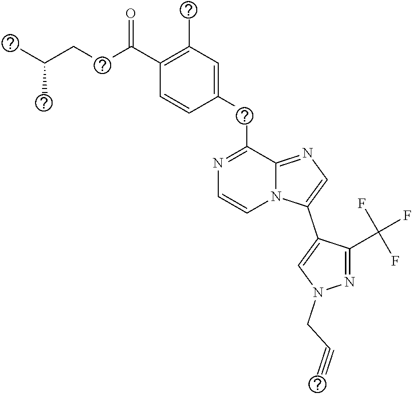 | 2-chloro-4-[[3- [1-(cyanomethyl)- 3- (trifluoromethyl) pyrazol-4- yl]imidazo[1,2- a]pyrazin-8- yl]amino]-N- [(2S)-2- hydroxypropyl] benzamide | 518.9 | 519.2 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with (S)-(+)-1-amino-2- propanol |
| 241 | 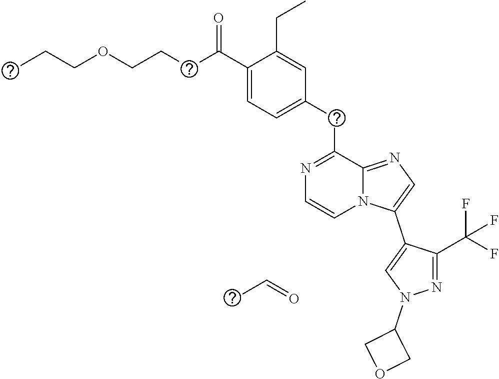 | N-[2-(2- aminoethoxy)ethyl]- 2-ethyl-4-[[3- [1-(oxetan-3-yl)- 3- (trifluoromethyl) pyrazol-4- yl]imidazo[1,2- a]pyrazin-8- yl]amino]benzamide formate | 558.6 | 559.5 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3-iodooxetane and subsequent acidic cleavage of the Boc group |
| 242 | 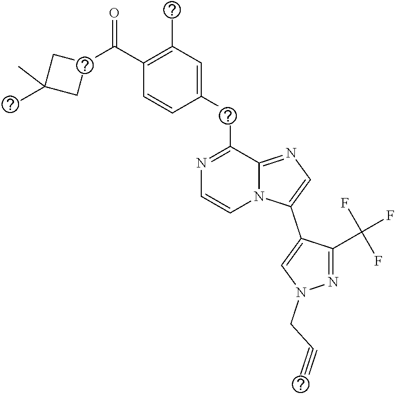 [SMILES: C#CCn1cc(-c2cnc3c(Cc4ccc(C(=O)C5CC(C)(C)C5)c(C)c4)nccn23)c(C(F)(F)F)n1] | 2-[4-[8-[3- chloro-4-(3- hydroxy-3- methylazetidine- 1- carbonyl)anilino] imidazo[1,2- a]pyrazin-3-yl]-3- (trifluoromethyl) pyrazol-1- yl]acetonitrile | 530.9 | 531.1 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with 3-methylazetidin- 3-ol |
| 243 | 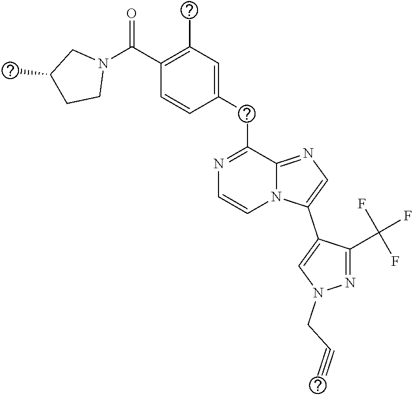 [SMILES: C#CCn1cc(-c2cnc3c(Cc4ccc(C(=O)N5CC[C@H](C)C5)c(C)c4)nccn23)c(C(F)(F)F)n1] | 2-[4-[8-[3- chloro-4-[(3S)-3- hydroxypyrrolidine- 1- carbonyl]anilino] imidazo[1,2- a]pyrazin-3-yl]-3- (trifluoromethyl) pyrazol-1- yl]acetonitrile | 530.9 | 531.1 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with (S)-3- hydroxypyrrolidine |
| 244 | 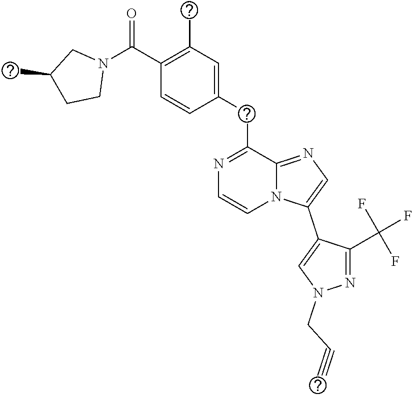 [SMILES: C#CCn1cc(-c2cnc3c(Cc4ccc(C(=O)N5CC[C@@H](C)C5)c(C)c4)nccn23)c(C(F)(F)F)n1] | 2-[4-[8-[3- chloro-4-[(3R)-3- hydroxypyrrolidine- 1- carbonyl]anilino] imidazo[1,2- a]pyrazin-3-yl]-3- (trifluoromethyl) pyrazol-1- yl]acetonitrile | 530.9 | 531.1 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with (R)-3- hydroxypyrrolidine |
| 245 | 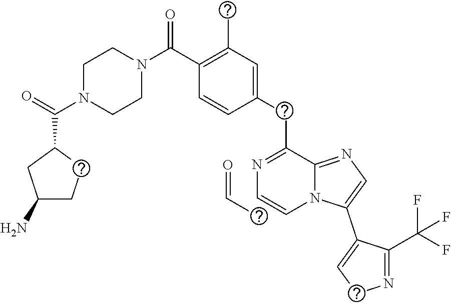 | [4-[(2R,4S)-4- aminopyrrolidine- 2- carbonyl]piperazin- 1-yl]-[2-chloro- 4-[[3-[3- (trifluoromethyl- 1H-pyrazol-4- yl]imidazo[1,2- a]pyrazin-8- yl]amino]phenyl] methanone formate | 603.0 | 603.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (2R,4S)- 1-tert-butoxycarbonyl-4-(tert- butoxycarbonylamino)pyrrolidine- 2-carboxylic acid and acidic Boc deprotection |
| 246 | 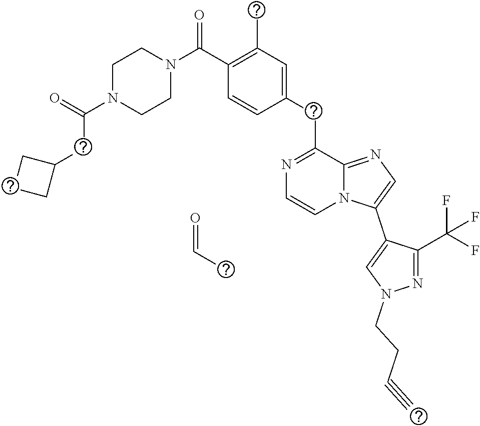 | N-(azetidin-3-yl)- 4-[2-chloro-4-[[3- [1-(2-cyanoethyl)- 3- (trifluoromethyl) pyrazol-4- yl]imidazo[1,2- a]pyrazin-8- yl]amino]benzoyl] piperazine-1- carboxamide formate | 642.0 | 642.2 | in analogy to the procedures for example 1/12: amide coupling of 2-chloro-4-[(3-iodoimidazo[1,2- a]pyrazin-8-yl)amino]benzoic acid (example 14, step 1) with BOC- piperazine, subsequent Suzuki reaction with 3-[4-(4,4,5,5- tetramethyl-1,3,2-dioxaborolan-2- yl)-3-(trifluoromethyl)pyrazol-1- yl]propanenitrile, acidic Boc deprotection, urea formation with 1-BOC-3-(amino)azetidine and subsequent acidic Boc deprotection |
| 247 | 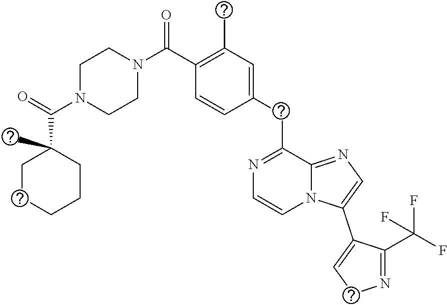 | [2-chloro-4-[[3- [3- (trifluoromethyl)- 1H-pyrazol-4- yl]imidazo[1,2- a]pyrazin-8- yl]amino]phenyl]- [4-[(3S)-3- hydroxypiperidine- 3- carbonyl]piperazin- 1-yl]methanone | 618.0 | 618.4 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 1-(tert- butoxycarbonyl)-3- hydroxypiperidine-3-carboxylic acid CAS [1396555-79-5] and acidic Boc deprotection (SFC chromatography) |
| 248 | 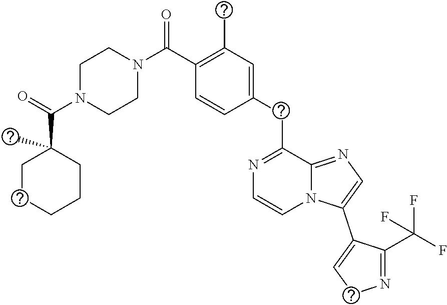 | [2-chloro-4-[[3- [3- (trifluoromethyl)- 1H-pyrazol-4- yl]imidazo[1,2- a]pyrazin-8- yl]amino]phenyl]- [4-[(3R)-3- hydroxypiperidine- 3- carbonyl]piperazin- 1-yl]methanone | 618.0 | 618.4 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 1-(tert- butoxycarbonyl)-3- hydroxypiperidine-3-carboxylic acid CAS [1396555-79-5] and acidic Boc deprotection (SFC chromatography) |
| 249 | 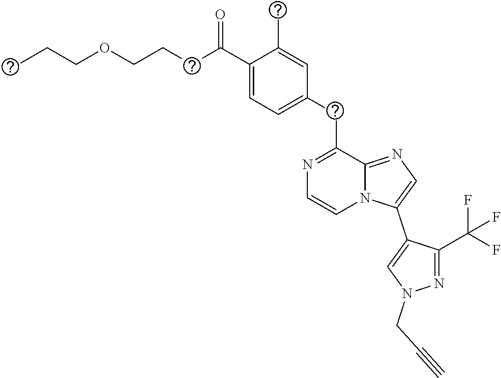 [SMILES: C#CCn1cc(-c2cnc3c(Cc4ccc(C(=O)CCCOCCC)c(C)c4)nccn23)c(C(F)(F)F)n1] | N-[2-(2- aminoethoxy)ethyl]- 2-chloro-4-[[3- [1-prop-2-ynyl-3- (trifluoromethyl) pyrazol-4- yl]imidazo[1,2- a]pyrazin-8- yl]amino]benzamide | 546.9 | 547.4 | in analogy to the procedure for example 1/2: amide formation from 2-chloro-4-[(3- iodoimidazo[1,2-a]pyrazin-8- yl)amino]benzoic acid (example 14, step 1) and tert-butyl (2-(2- aminoethoxy)ethyl)carbamate, followed by Suzuki reaction with (3-(trifluoromethyl)-1H-pyrazol-4- yl)boronic acid, alkylation with 3- bromoprop-1-yne and subsequent acidic cleavage of the Boc group |
| 250 | 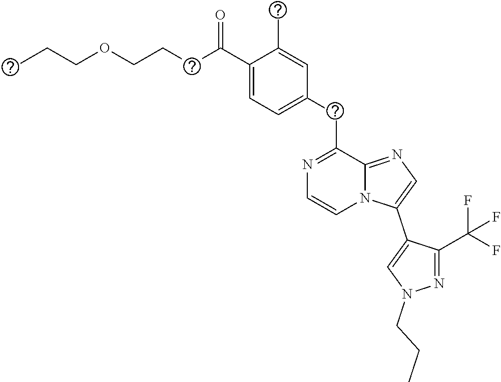 [SMILES: CCCOCCCC(=O)c1ccc(Cc2nccn3c(-c4cn(CCC)nc4C(F)(F)F)cnc23)cc1C] | N-[2-(2- aminoethoxy)ethyl]- 2-chloro-4-[[3- [1-(2-fluoroethyl)- 3- (trifluoromethyl) pyrazol-4- yl]imidazo[1,2- a]pyrazin-8- yl]amino]benzamide | 554.9 | 555.4 | in analogy to the procedure for example 1/2: amide formation from 2-chloro-4-[(3- iodoimidazo[1,2-a]pyrazin-8- yl)amino]benzoic acid (example 14, step 1) and tert-butyl (2-(2- aminoethoxy)ethyl)carbamate, followed by Suzuki reaction with (3-(trifluoromethyl)-1H-pyrazol-4- yl)boronic acid, alkylation with 1- bromo-2-fluoroethane and subsequent acidic cleavage of the Boc group |
| 251 | 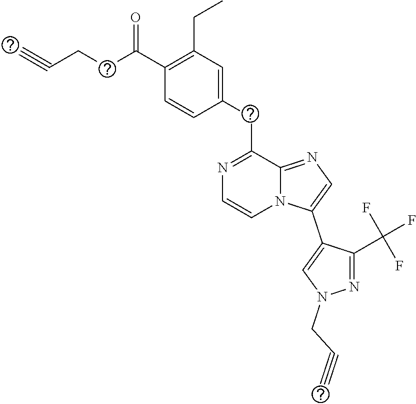 [SMILES: C#CCCC(=O)c1ccc(Cc2nccn3c(-c4cn(CC#C)nc4C(F)(F)F)cnc23)cc1CC] | N- (cyanomethyl)-4- [[3-[1- (cyanomethyl)-3- (trifluoromethyl) pyrazol-4- yl]imidazo[1,2- a]pyrazin-8- yl]amino]-2- ethylbenzamide | 493.5 | 494.1 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with 2- aminoacetonitrile |
| 252 | 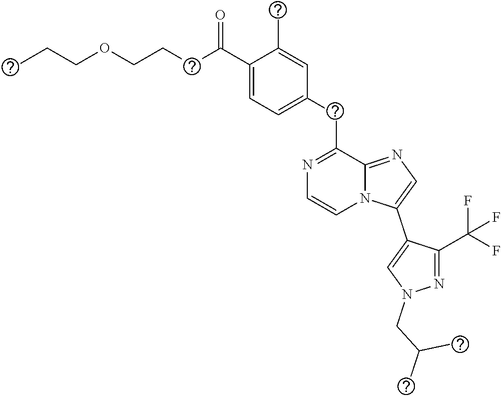 [SMILES: CCCOCCCC(=O)c1ccc(Cc2nccn3c(-c4cn(CC(C)C)nc4C(F)(F)F)cnc23)cc1C] | N-[2-(2- aminoethoxy)ethyl]- 2-chloro-4-[[3- [1-(2,2- difluoroethyl)-3- (trifluoromethyl) pyrazol-4- yl]imidazo[1,2- a]pyrazin-8- yl]amino] benzamide | 572.9 | 573.5 | in analogy to the procedure for example 1/2: amide formation from 2-chloro-4-[(3- iodoimidazo[1,2-a]pyrazin-8- yl)amino]benzoic acid (example 14, step 1) and tert-butyl (2-(2- aminoethoxy)ethyl)carbamate, followed by Suzuki reaction with (3-(trifluoromethyl)-1H-pyrazol-4- yl)boronic acid, alkylation with 1,1-difluoro-2-iodo-ethane and subsequent acidic cleavage of the Boc group |
| 253 | 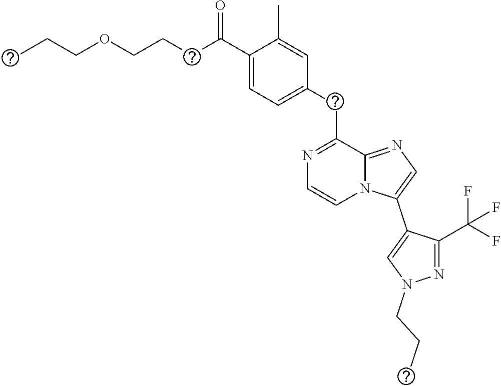 [SMILES: CCCOCCCC(=O)c1ccc(Cc2nccn3c(-c4cn(CCC)nc4C(F)(F)F)cnc23)cc1C] | N-[2-(2- aminoethoxy)ethyl]- 4-[[3-[1-(2- fluoroethyl)-3- (trifluoromethyl) pyrazol-4- yl]imidazo[1,2- a]pyrazin-8- yl]amino]-2- methylbenzamide | 534.5 | 535.5 | in analogy to the procedure described for example 2: alkylation of tert-butyl N-[2-[2- [[2-methyl-4-[[3-[3- (trifluoromethyl)-1H-pyrazol-4- yl]imidazo[1,2-a]pyrazin-8- yl]amino]benzoyl]amino]ethoxy] ethyl]carbamate (example 16, step 3) with 1-bromo-2-fluoroethane and subsequent acidic Boc deprotection |
| 254 | 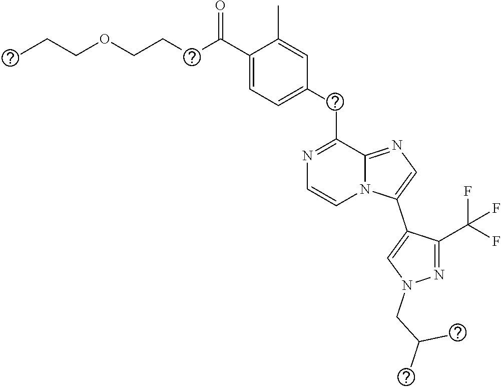 [SMILES: CCCOCCCC(=O)c1ccc(Cc2nccn3c(-c4cn(CC(C)C)nc4C(F)(F)F)cnc23)cc1C] | N-[2-(2- aminoethoxy)ethyl]- 4-[[3-[1-(2,2- difluoroethyl)-3- (trifluoromethyl) pyrazol-4- yl]imidazo[1,2- a]pyrazin-8- yl]amino]-2- methylbenzamide | 552.5 | 553.4 | in analogy to the procedure described for example 2: alkylation of tert-butyl N-[2-[2- [[2-methyl-4-[[3-[3- (trifluoromethyl)-1H-pyrazol-4- yl]imidazo[1,2-a]pyrazin-8- yl]amino]benzoyl]amino]ethoxy] ethyl]carbamate (example 16, step 3) with 1,1-difluoro-2-iodo-ethane and subsequent acidic Boc deprotection |
| 255 | 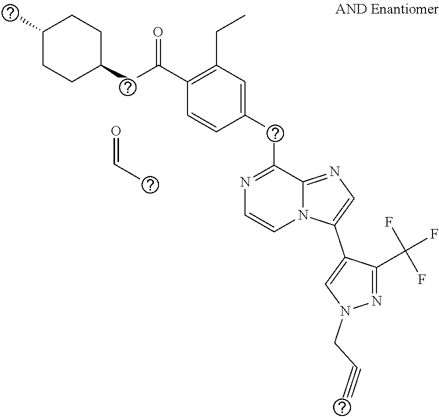 | N-(4- aminocyclohexyl)- 4-[[3-[1- (cyanomethyl)-3- (trifluoromethyl) pyrazol-4- yl]imidazo[1,2- a]pyrazin-8- yl]amino]-2- ethylbenzamide formate | 551.6 | 552.3 | in analogy to procedures for example 14: amide coupling of 2- chloro-4-[[3-[1-(cyanomethyl)-3- (trifluoromethyl)pyrazol-4- yl]imidazo[1,2-a]pyrazin-8- yl]amino]benzoic acid (example 14, step 2) with trans-N-Boc-1,4- cyclohexandiamine and acidic Boc deprotection |
| 256 | 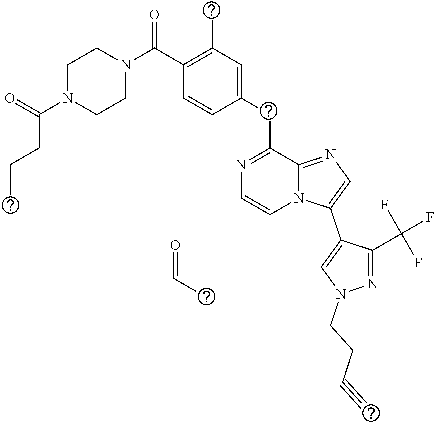 | 3-[4-[8-[4-[4-(3- aminopropanoyl) piperazine-1- carbonyl]-3- chloroanilino] imidazo[1,2- a]pyrazin-3-yl]-3- (trifluoromethyl) pyrazol-1- yl]propanenitrile formate | 615.0 | 615.2 | in analogy to the procedures for example 1/12: amide coupling of 2-chloro-4-[(3-iodoimidazo[1,2- a]pyrazin-8-yl)amino]benzoic acid (example 14, step 1) with BOC- piperazine, subsequent Suzuki reaction with 3-[4-(4,4,5,5- tetramethyl-1,3,2-dioxaborolan-2- yl)-3-(trifluoromethyl)pyrazol-1- yl]propanenitrile, acidic Boc deprotection, amide formation with Boc-BETA-ALA-OH and subsequent acidic Boc deprotection |
| 257 | 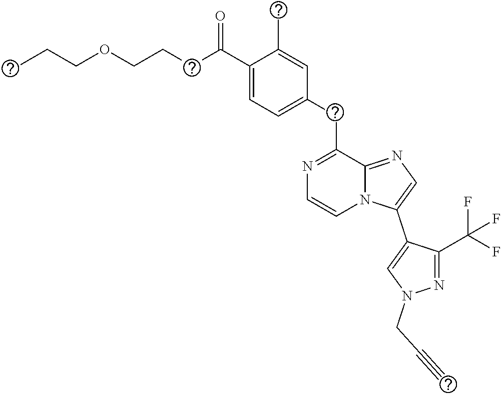 [SMILES: C#CCn1cc(-c2cnc3c(Cc4ccc(C(=O)CCCOCCC)c(C)c4)nccn23)c(C(F)(F)F)n1] | N-[2-(2- aminoethoxy)ethyl]- 2-chloro-4-[[3- [1-(cyanomethyl)- 3- (trifluoromethyl) pyrazol-4- yl]imidazo[1,2- a]pyrazin-8- yl]amino]benzamide | 547.9 | 548.4 | in analogy to the procedure for example 1/2: amide formation from 2-chloro-4-[(3- iodoimidazo[1,2-a]pyrazin-8- yl)amino]benzoic acid (example 14, step 1) and tert-butyl (2-(2- aminoethoxy)ethyl)carbamate, followed by Suzuki reaction with (3-(trifluoromethyl)-1H-pyrazol-4- yl)boronic acid, alkylation with 2- bromoacetonitrile and subsequent acidic cleavage of the Boc group |
| 258 | 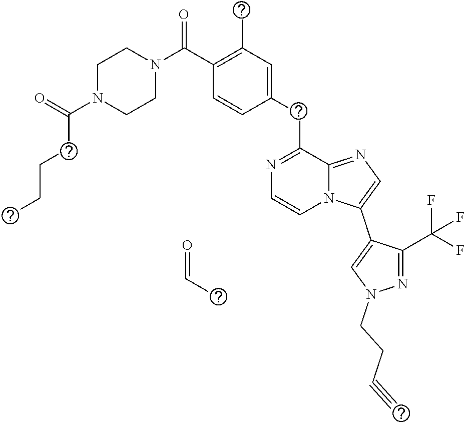 | N-(2- aminoethyl)-4-[2- chloro-4-[[3-[1- (2-cyanomethyl)-3- (trifluoromethyl) pyrazol-4- yl]imidazo[1,2- a]pyrazin-8- yl]amino]benzoyl] piperazine-1- carboxamide formate | 630.0 | 630.2 | in analogy to the procedures for example 1/12: amide coupling of 2-chloro-4-[(3-iodoimidazo[1,2- a]pyrazin-8-yl)amino]benzoic acid (example 14, step 1) with Boc- piperazine, subsequent Suzuki reaction with 3-[4-(4,4,5,5- tetramethyl-1,3,2-dioxaborolan-2- yl)-3-(trifluoromethyl)pyrazol-1- yl]propanenitrile, acidic Boc deprotection, urea formation with N-Boc-ethylenediamine and subsequent acidic Boc deprotection |
| 259 | 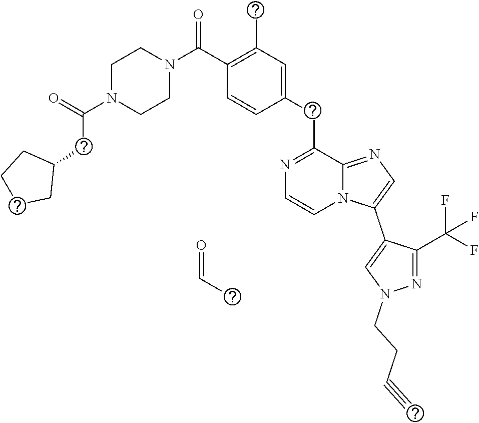 | 4-[2-chloro-4- [[3-[1-(2- cyanoethyl)-3- (trifluoromethyl) pyrazol-4- yl]imidazo[1,2- a]pyrazin-8- yl]amino]benzoyl]- N-[(3S)- pyrrolidin-3- yl]piperazine-1- carboxamide formate | 656.1 | 656.3 | in analogy to the procedures for example 1/12: amide coupling of 2-chloro-4-[(3-iodoimidazo[1,2- a]pyrazin-8-yl)amino]benzoic acid (example 14, step 1) with Boc- piperazine, subsequent Suzuki reaction with 3-[4-(4,4,5,5- tetramethyl-1,3,2-dioxaborolan-2- yl)-3-(trifluoromethyl)pyrazol-1- yl]propanenitrile, acidic Boc deprotection, urea formation with (S)-3-amino-1-N-Boc-pyrrolidine and subsequent acidic Boc deprotection |
| 260 | 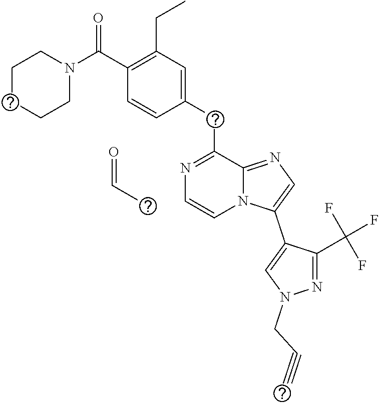 | 2-[4-[8-[3-ethyl- 4-(piperazine-1- carbonyl)anilino] imidazo[1,2- a]pyrazin-3-yl]-3- (trifluoromethyl) pyrazol-1- yl]acetonitrile formate | 523.5 | 524.2 | in analogy to the procedures for example 6: amide formation with 2-ethyl-4-[[3-[3-(trifluoromethyl)- 1H-pyrazol-4-yl]imidazo[1,2- a]pyrazin-8-yl]amino]benzoic acid, hydrochloric acid (example 3, step 2) and 1-Boc-piperazine and subsequent acidic Boc deprotection |
| 261 | 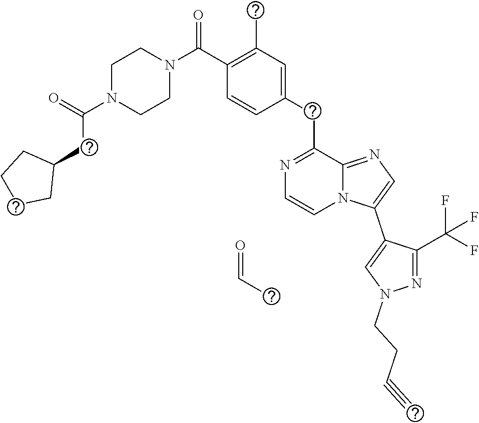 | 4-[2-chloro-4- [[3-[1-(2- cyanoethyl)-3- pyrazol-4- yl]imidazo[1,2- a]pyrazin-8- yl]amino]benzoyl]- N-[(3R)- pyrrolidin-3- yl]piperazine-1- carboxamide formate | 656.1 | 656.3 | in analogy to the procedures for example 1/12: amide coupling of 2-chloro-4-[(3-iodoimidazo[1,2- a]pyrazin-8-yl)amino]benzoic acid (example 14, step 1) with Boc- piperazine, subsequent Suzuki reaction with 3-[4-(4,4,5,5- tetramethyl-1,3,2-dioxaborolan-2- yl)-3-(trifluoromethyl)pyrazol-1- yl]propanenitrile, acidic Boc deprotection, urea formation with (R)-3-amino-1-N-Boc-pyrrolidine and subsequent acidic Boc deprotection |
| 262 | 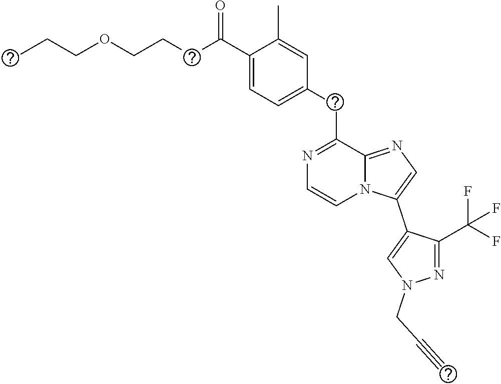 [SMILES: C#CCn1cc(-c2cnc3c(Cc4ccc(C(=O)CCCOCCC)c(C)c4)nccn23)c(C(F)(F)F)n1] | N-[2-(2- aminoethoxy)ethyl]- 4-[[3-[1- (cyanomethyl)-3- (trifluoromethyl) pyrazol-4- yl]imidazo[1,2- a]pyrazin-8- yl]amino]-2- methylbenzamide | 527.5 | 528.4 | in analogy to the procedure described for example 2: alkylation of tert-butyl N-[2-[2- [[2-methyl-4-[[3-[3- (trifluoromethyl)-1H-pyrazol-4- yl]imidazo[1,2-a]pyrazin-8- yl]amino]benzoyl]amino]ethoxy] ethyl]carbamate (example 16, step 3) with 2-bromoacetonitrile and subsequent acidic Boc deprotection |
| 263 | 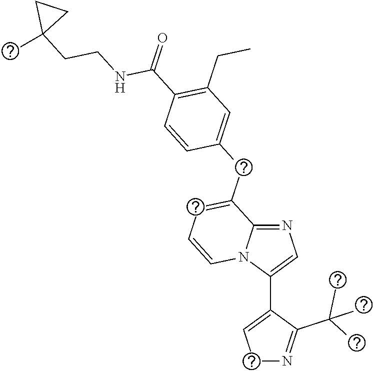 [SMILES: CCc1cc(Cc2cccn3c(C4=CCN=C4C(C)(C)C)cnc23)ccc1C(=O)NCCC1(C)CC1] | N-[2-(1- aminocyclopropyl) ethyl]-2-ethyl-4- [[3-[3- (trifluoromethyl)- 1H-pyrazol-4- yl]imidazo[1,2- a]pyrazin-8- yl]amino]benzamide | 498.5 | 499.4 | in analogy to the procedures for example 1: amide coupling of 2- ethyl-4-[(3-iodoimidazo[1,2- a]pyrazin-8-yl)amino]benzoic acid, hydrochloride (example 1, step 2) with tert-butyl (1-(2- aminoethyl)cyclopropyl)carbamate hydrochloride, subsequent Suzuki reaction with (3-(trifluoromethyl)- 1H-pyrazol-4-yl)boronic acid and subsequent acidic Boc deprotection |
| 264 | 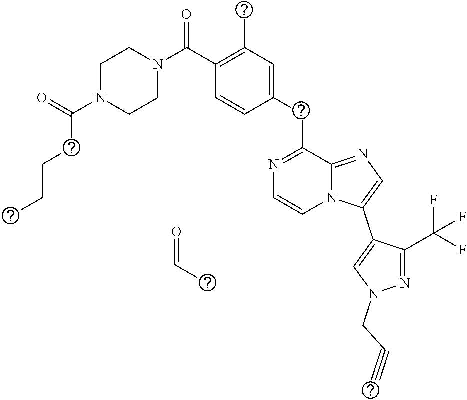 | N-(2- aminoethyl)-4-[2- chloro-4-[[3-[1- (cyanomethyl)-3- (trifluoromethyl) pyrazol-4- yl]imidazo[1,2- a]pyrazin-8- yl]amino]benzoyl] piperazine-1- carboxamide formate | 616.0 | 616.3 | in analogy to the procedures for example 12: urea formation with 2-[4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and N-Boc-ethylenediamine followed by acidic Boc deprotection |
| 265 | 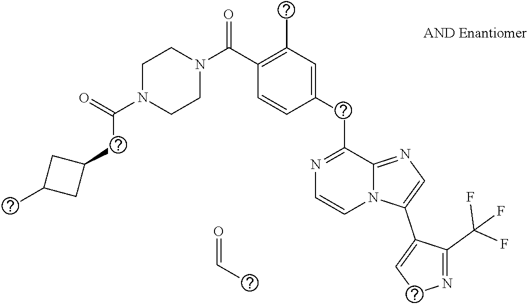 | N-(3- aminocyclobutyl)- 4-[2-chloro-4-[[3- [3- (trifluoromethyl)- 1H-pyrazol-4- yl]imidazo[1,2- a]pyrazin-8- yl]amino]benzoyl] piperazine-1- carboxamide formate | 603.0 | 603.1 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl 3- aminocyclobutylcarbamate followed by acidic Boc deprotection |
| 266 | 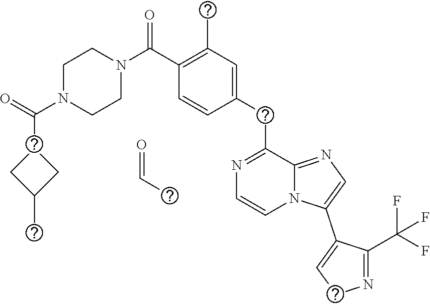 | [4-(3- aminoazetidine-1- carbonyl)piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 589.0 | 589.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and 3-N-Boc-amino-azetidine followed by Boc deprotection with trimethylsilyl trifluormethanesulfonate |
| 267 | 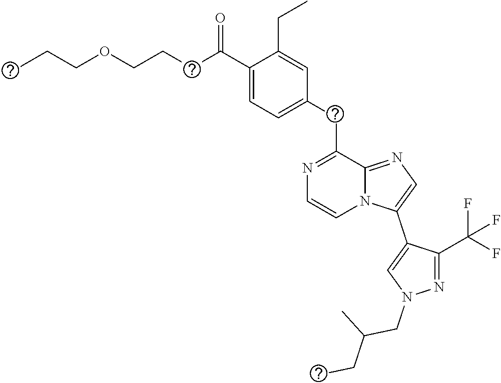 [SMILES: CCCOCCCC(=O)c1ccc(Cc2nccn3c(-c4cn(CC(C)CC)nc4C(F)(F)F)cnc23)cc1CC] | N-[2-(2- aminoethoxy)ethyl]- 2-ethyl-4-[[3- [1-(3-hydroxy-2- methylpropyl)-3- (trifluoromethyl) pyrazol-4- yl]imidazo[1,2- a]pyrazin-8- yl]amino]benzamide | 574.6 | 575.6 | in analogy to the procedure from example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-bromo-2-methylpropan-1-ol and subsequent acidic cleavage of the Boc group |
| 268 | 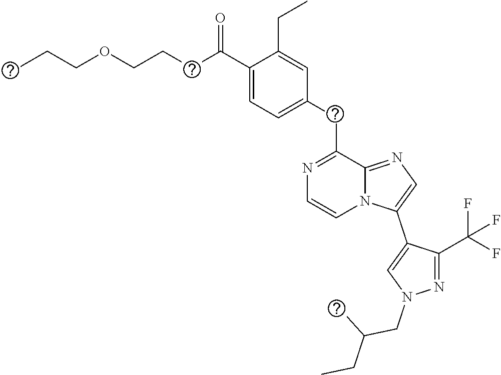 [SMILES: CCCOCCCC(=O)c1ccc(Cc2nccn3c(-c4cn(CC(C)CC)nc4C(F)(F)F)cnc23)cc1CC] | N-[2-(2- aminoethoxy)ethyl]- 2-ethyl-4-[[3- [1-(2- hydroxybutyl)-3- (trifluoromethyl) pyrazol-4- yl]imidazo[1,2- a]pyrazin-8- yl]amino]benzamide | 574.6 | 575.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 1-bromobutan-2-ol and subsequent acidic cleavage of the Boc group |
| 269 | 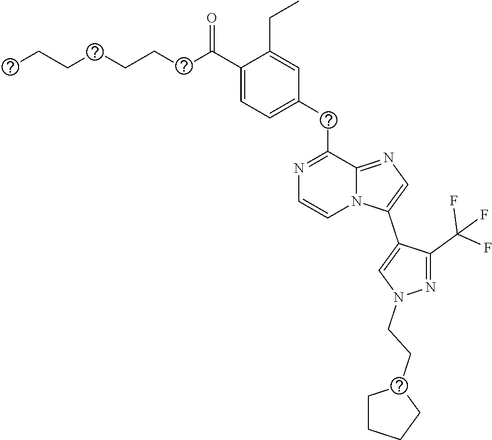 [SMILES: CCCCCCCC(=O)c1ccc(Cc2nccn3c(-c4cn(CCC5CCCC5)nc4C(F)(F)F)cnc23)cc1CC] | N-[2-(2- aminoethoxy)ethyl]- 2-ethyl-4-[[3- [1-(2-pyrrolidin- 1-ylethyl)-3- (trifluoromethyl) pyrazol-4- yl]imidazo[1,2- a]pyrazin-8- yl]amino]benzamide | 599.7 | 600.6 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 1-(2-iodoethyl)pyrrolidine and subsequent acidic cleavage of the Boc group |
| 270 | 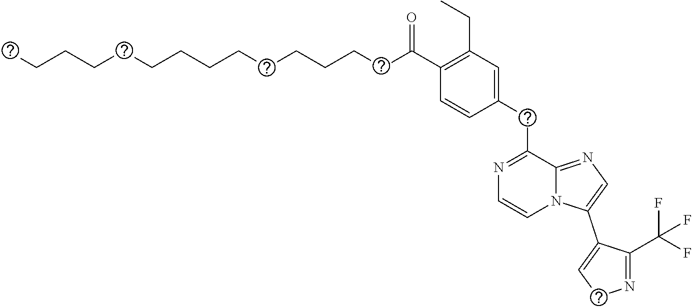 [SMILES: CCCCCCCCCCCCCCC(=O)c1ccc(Cc2nccn3c(C4=CCN=C4C(F)(F)F)cnc23)cc1CC] | N-[3-[4-(3- aminopropylamino) butylamino] propyl]-2-ethyl-4-[[3- [3- (trifluoromethyl)- 1H-pyrazol-4- yl]imidazo[1,2- a]pyrazin-8- yl]amino]benzamide | 600.7 | 601.6 | in analogy to the procedures for example 1: amide coupling with 2- ethyl-4-[(3-iodoimidazo[1,2- a]pyrazin-8-yl)amino]benzoic acid, hydrochloride (example 1, step 2) with tert-butyl (3-aminopropyl)(4- ((tert-butoxycarbonyl)(3-((tert- butoxycarbonyl)amino)propyl) amino)butyl)carbamate, subsequent Suzuki reaction with (3- (trifluoromethyl)-1H-pyrazol-4- yl)boronic acid and acidic Boc deprotection |
| 271 | 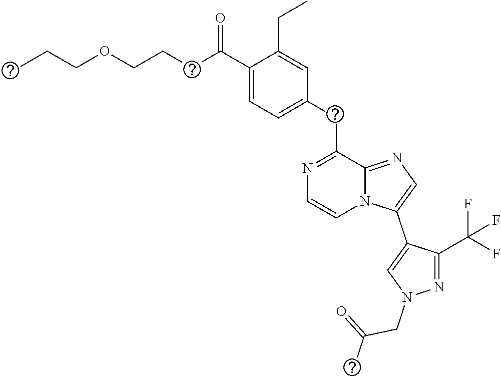 [SMILES: CCCOCCCC(=O)c1ccc(Cc2nccn3c(-c4cn(CC(C)=O)nc4C(F)(F)F)cnc23)cc1CC] | 2-[4-[8-[4-[2-(2- aminoethoxy) ethylcarbamoyl]-3- ethylanilino]imidazo [1,2-a]pyrazin- 3-yl]-3- (trifluoromethyl) pyrazol-1-yl]acetic acid | 560.5 | 557.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy ethyl]carbamate (example 2, step 1) with 2-bromoacetic acid and subsequent acidic cleavage of the Boc group |
| 272 | 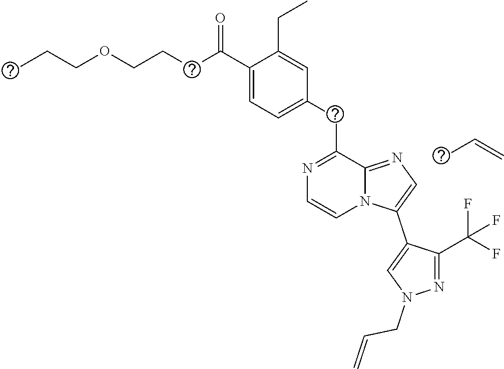 | N-[2-(2- aminoethoxy)ethyl]- 2-ethyl-4-[[3- [1-prop-2-enyl-3- (trifluoromethyl) pyrazol-4- yl]imidazo[1,2- a]pyrazin-8- yl]amino]benzamide formate | 542.6 | 543.4 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-bromoprop-1-ene and subsequent acidic cleavage of the Boc group |
| 273 | 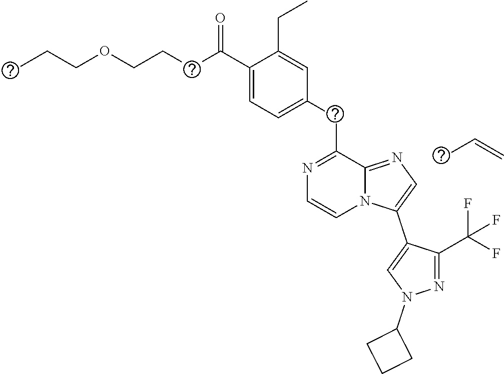 | N-[2-(2- aminoethoxy)ethyl]- 4-[[3-[1- cyclobutyl-3- (trifluoromethyl) pyrazol-4- yl]imidazo[1,2- a]pyrazin-8- yl]amino]-2- ethylbenzamide formate | 556.6 | 557.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with iodocyclobutane and subsequent acidic cleavage of the Boc group |
| 274 | 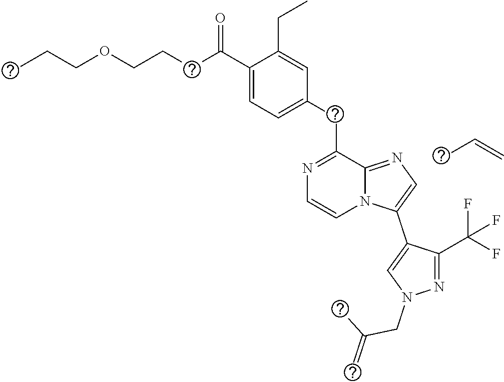 | N-[2-(2- aminoethoxy)ethyl]- 4-[[3-[1-(2- chloroprop-2- enyl)-3- (trifluoromethyl) pyrazol-4- yl]imidazo[1,2- a]pyrazin-8- yl]amino]-2- ethylbenzamide formate | 577.0 | 577.4 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-bromoprop-1-yne and subsequent acidic cleavage of the Boc group |
| 275 | 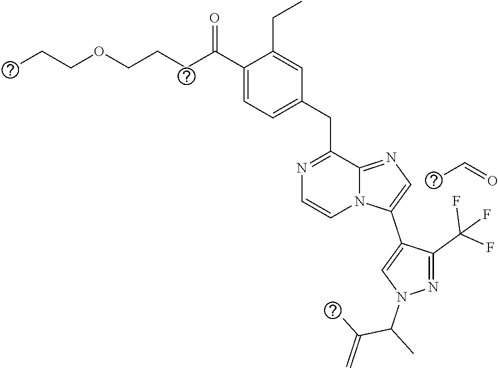 | N-[2-(2- aminoethoxy)ethyl]- 4-[[3-[1-(3- chlorobut-3-en-2- yl)-3- (trifluoromethyl) pyrazol-4- yl]imidazo[1,2- a]pyrazin-8- yl]amino]-2- ethylbenzamide formate | 591.0 | 591.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-bromobut-1-yne and subsequent acidic cleavage of the Boc group |
| 276 | 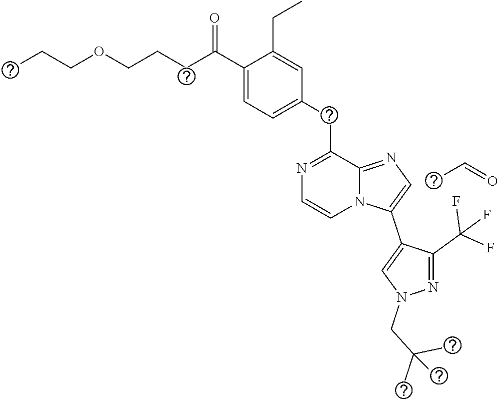 | N-[2-(2- aminoethoxy)ethyl]- 2-ethyl-4-[[3- [1-(2,2,2- trifluoroethyl)-3- (trifluoromethyl) pyrazol-4- yl]imidazo[1,2- a]pyrazin-8- yl]amino]benzamide formate | 584.5 | 585.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 1,1,1-trifluoro-3-iodopropane and subsequent acidic cleavage of the Boc group |
| 277 | 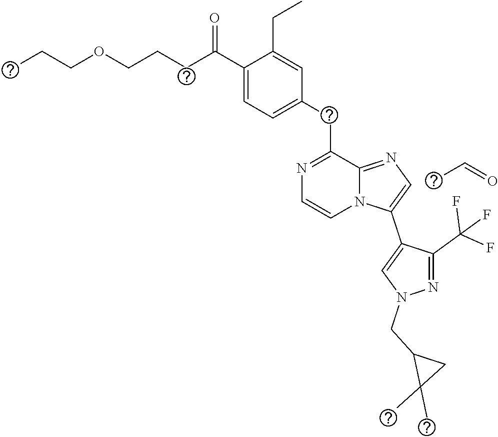 | N-[2-(2- aminoethoxy)ethyl]- 4-[[3-[1-[(2,2- difluorocyclopropyl) methyl]-3- (trifluoromethyl) pyrazol-4- yl]imidazo[1,2- a]pyrazin-8- yl]amino]-2- ethylbenzamide formate | 592.6 | 593.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 2-(bromomethyl)-1,1- difluorocyclopropane and subsequent acidic cleavage of the Boc group |
| 278 | 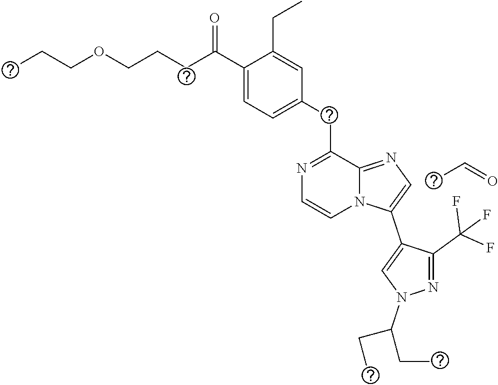 | N-[2-(2- aminoethoxy)ethyl]- 4-[[3-[1-(1- chloro-3- hydroxypropan-2- yl)-3- (trifluoromethyl) pyrazol-4- yl]imidazo[1,2- a]pyrazin-8- yl]amino]-2- ethylbenzamide formate | 595.0 | 595.5 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-iodooxetane and subsequent acidic cleavage of the Boc group |
| 279 | 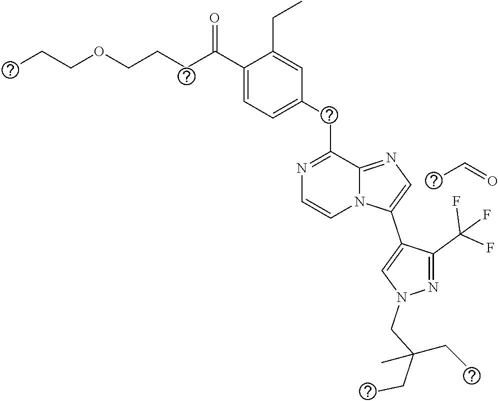 | N-[2-(2- aminoethoxy)ethyl]- 4-[[3-[1-[2- (chloromethyl)-3- hydroxy-2- methylpropyl]-3- (trifluoromethyl) pyrazol-4- yl]imidazo[1,2- a]pyrazin-8- yl]amino]-2- ethylbenzamide formate | 623.1 | 623.6 | in analogy to the procedure for example 2: alkylation of tert-butyl N-[2-[2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1) with 3-(bromomethyl)-3- methyloxetane and subsequent acidic cleavage of the Boc group |
| 280 | 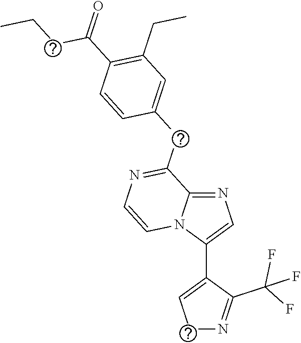 [SMILES: CCCC(=O)c1ccc(Cc2nccn3c(C4=CCN=C4C(F)(F)F)cnc23)cc1CC] | N,2-diethyl-4- [[3-[3- (trifluoromethyl)- 1H-pyrazol-4- yl]imidazo[1,2- a]pyrazin-8- yl]amino]benzamide | 443.4 | 444.4 | in analogy to the procedure for example 1: amide coupling of 2- ethyl-4-[(3-iodoimidazo[1,2- a]pyrazin-8-yl)amino]benzoic acid, hydrochloride (example 1, step 2) with ethyl amine and subsequent Suzuki reaction with (3- (trifluoromethyl)-1H-pyrazol-4- yl)boronic acid |
| 281 | 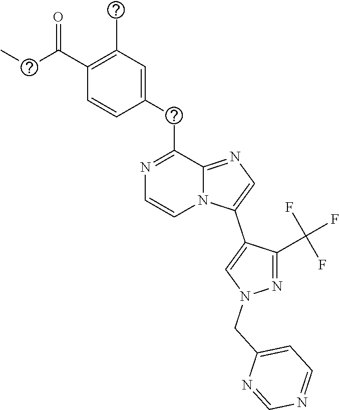 [SMILES: CCC(=O)c1ccc(Cc2nccn3c(-c4cn(Cc5ccncn5)nc4C(F)(F)F)cnc23)cc1C] | 2-chloro-N- methyl-4-[[3-[1- (pyrimidin-4- ylmethyl)-3- (trifluoromethyl) pyrazol-4- yl]imidazo[1,2- a]pyrazin-8- yl]amino]benzamide | 527.9 | 528.3 | in analogy to the procedure for example 15: alkylation of 2- chloro-N-methyl-4-[[3-[3- (trifluoromethyl)-1H-pyrazol-4- yl]imidazo[1,2-a]pyrazin-8- yl]amino]benzamide (example 10, step 2) with pyrimidin-4- ylmethanol [CAS#33581-98-5] |
| 282 | 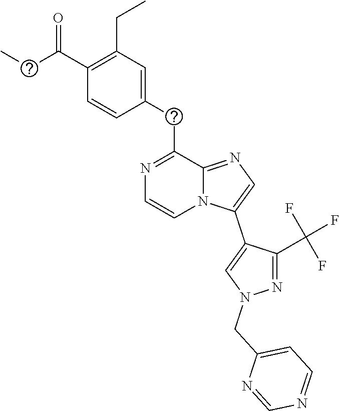 [SMILES: CCC(=O)c1ccc(Cc2nccn3c(-c4cn(Cc5ccncn5)nc4C(F)(F)F)cnc23)cc1CC] | 2-ethyl-N- methyl-4-[[3-[1- (pyrimidin-4- ylmethyl)-3- (trifluoromethyl) pyrazol-4- yl]imidazo[1,2- a]pyrazin-8- yl]amino]benzamide | 521.5 | 522.4 | in analogy to the procedure for example 15: alkylation of 2-ethyl- N-methyl-4-[[3-[3- (trifluoromethyl)-1H-pyrazol-4- yl]imidazo[1,2-a]pyrazin-8- yl]amino]benzamide (example 10, step 2) with pyrimidin-4- ylmethanol [CAS#33581-98-5] |
| 283 | 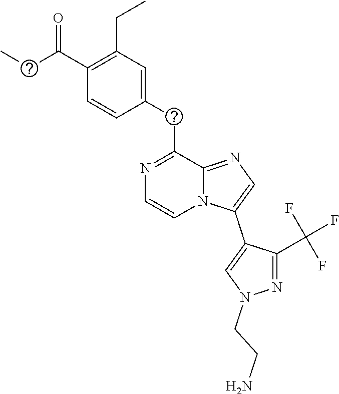 [SMILES: CCC(=O)c1ccc(Cc2nccn3c(-c4cn(CCN)nc4C(F)(F)F)cnc23)cc1CC] | 4-[[3-[1-(2- aminoethyl)-3- (trifluoromethyl) pyrazol-4- yl]imidazo[1,2- a]pyrazin-8- yl]amino]-2-ethyl- N- methylbenzamide | 472.5 | 473.3 | in analogy to the procedure for example 15: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with tert-butyl (2- hydroxyethyl)carbamate [CAS#26690-80-2] and subsequent acidic Boc deprotection |
| 284 | 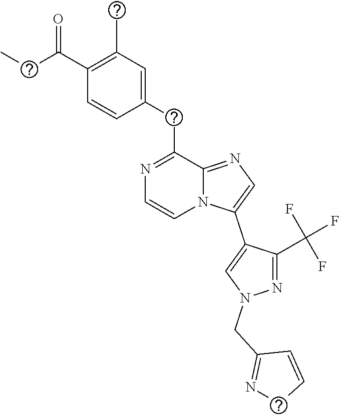 [SMILES: CCC(=O)c1ccc(Cc2nccn3c(-c4cn(CC5=NCC=C5)nc4C(F)(F)F)cnc23)cc1C] | 2-chloro-N- methyl-4-[[3-[1- (1H-pyrazol-3- ylmethyl)-3- (trifluoromethyl) pyrazol-4- yl]imidazo[1,2- a]pyrazin-8- yl]amino]benzamide | 515.9 | 516.3 | in analogy to the procedure for example 15: alkylation of 2- chloro-N-methyl-4-[[3-[3- (trifluoromethyl)-1H-pyrazol-4- yl]imidazo[1,2-a]pyrazin-8- yl]amino]benzamide (example 10, step 2) with (1H-pyrazol-3- yl)methanol [CAS#23585-49-1] |
| 285 | 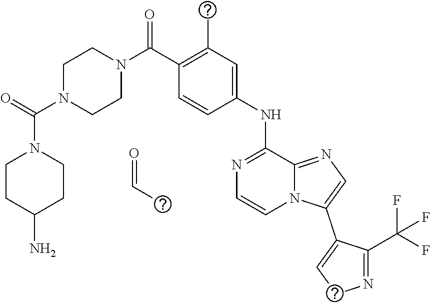 | [4-(4- aminopiperidine- 1- carbonyl)piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 617.0 | 617.3 | in analogy to the procedure for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and 4-Boc-aminopiperidine followed by acidic Boc deprotection |
| 286 | 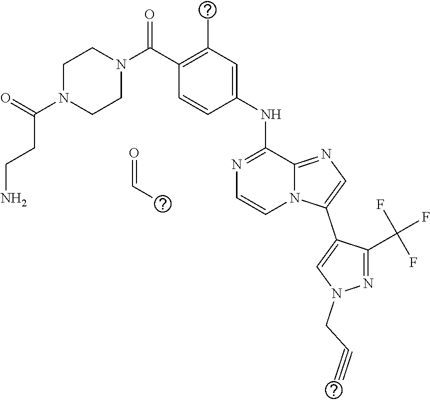 | 2-[4-[8-[4-[4-(3- aminopropanoyl) piperazine-1- carbonyl]-3- chloroanilino] imidazo[1,2- a]pyrazin-3-yl]-3- (trifluoromethyl) pyrazol-1- yl]acetonitrile formate | 601.0 | 601.2 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and Boc-BETA-ALA-OH followed by acidic Boc deprotection |
| 287 | 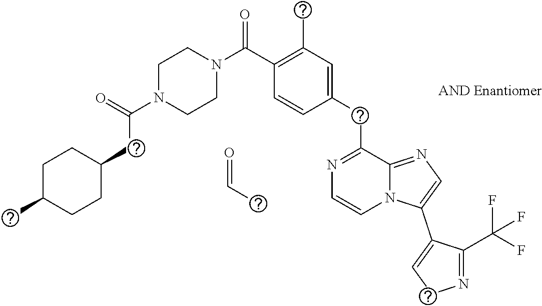 | N-(4- aminocyclohexyl)- 4-[2-chloro-4- [[3-[3- (trifluoromethyl)- 1H-pyrazol-4- yl]imidazo[1,2- a]pyrazin-8- yl]amino]benzoyl] piperazine-1- carboxamide formate | 631.1 | 631.2 | in analogy to the procedure for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and 1-N-BOC-cis-1,4- cyclohexyldiamine followed by acidic Boc deprotection |
| 288 | 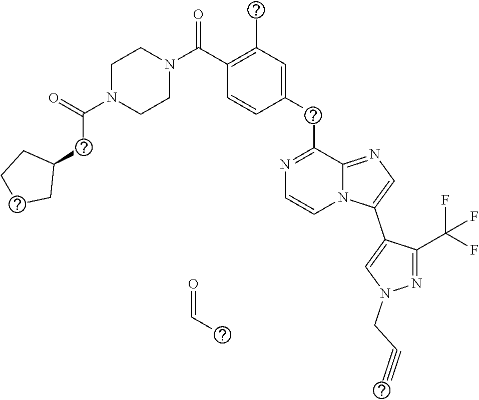 | 4-[2-chloro-4- [[3-[1- (cyanomethyl)-3- (trifluoromethyl) pyrazol-4- yl]imidazo[1,2- a]pyrazin-8- yl]amino]benzoyl]- N-[(3R)- pyrrolidin-3- yl]piperazine-1- carboxamide formate | 642.0 | 642.3 | in analogy to the procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and (R)-(+)-1-Boc-3- aminopyrrolidine followed by acidic Boc deprotection |
| 289 | 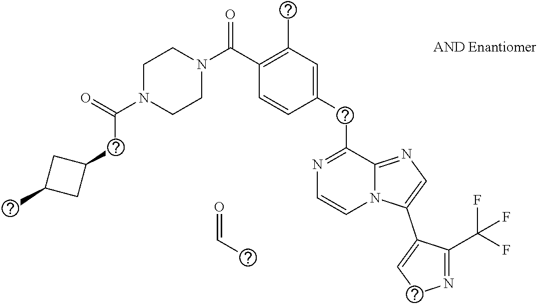 | N-(3- aminocyclobutyl)- 4-[2-chloro-4-[[3- [3- (trifluoromethyl)- 1H-pyrazol-4- yl]imidazo[1,2- a]pyrazin-8- yl]amino]benzoyl] piperazine-1- carboxamide formate | 603.0 | 603.2 | in analogy to the procedure for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl N-(3- aminocyclobutyl)carbamate followed by acidic Boc deprotection |
| 290 | 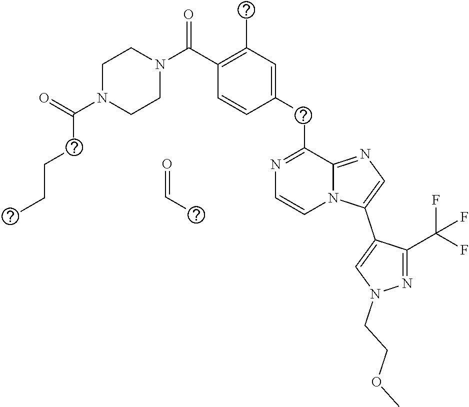 | N-(2- aminoethyl)-4-[2- chloro-4-[[3-[1- (2-methoxyethyl)- 3- (trifluoromethyl) pyrazol-4- yl]imidazo[1,2- a]pyrazin-8- yl]amino]benzoyl] piperazine-1- carboxamide formate | 635.0 | 635.2 | in analogy to the procedures for example 1/12: amide coupling of 2-chloro-4-[(3-iodoimidazo[1,2- a]pyrazin-8-yl)amino]benzoic acid (example 14, step 1) with Boc- piperazine, subsequent Suzuki reaction with 3-[4-(4,4,5,5- tetramethyl-1,3,2-dioxaborolan-2- yl)-3-(trifluoromethyl)pyrazol-1- yl]propanenitrile, acidic Boc deprotection, amide formation with Boc-BETA-ALA-OH and subsequent acidic Boc deprotection |
| 291 | 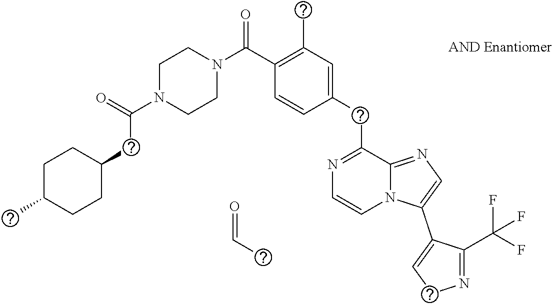 | N-(4- aminocyclohexyl)- 4-[2-chloro-4- [[3-[3- (trifluoromethyl)- 1H-pyrazol-4- yl]imidazo[1,2- a]pyrazin-8- yl]amino]benzoyl] piperazine-1- carboxamide formate | 631.1 | 631.3 | in analogy to the procedure for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and trans-N-Boc-1,4- cyclohexandiamine followed by acidic Boc deprotection |
| 292 | 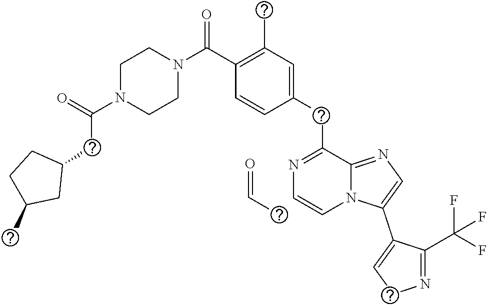 | N-[(1S,3S)-3- aminocyclopentyl]- 4-[2-chloro-4- [[3-[3- (trifluoromethyl)- 1H-pyrazol-4- yl]imidazo[1,2- a]pyrazin-8- yl]amino]benzoyl] piperazine-1- carboxamide formate | 617.0 | 617.3 | in analogy to the procedure for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl N-[(1S,3S)-3- aminocyclopentyl]carbamate followed by acidic Boc deprotection |
| 293 | 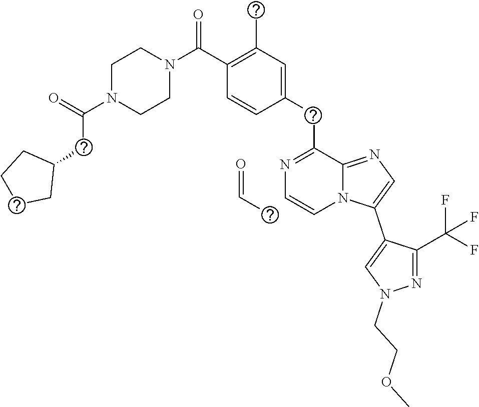 | 4-[2-chloro-4- [[3-[1-(2- methoxyethyl)-3- (trifluoromethyl) pyrazol-4- yl]imidazo[1,2- a]pyrazin-8- yl]amino]benzoyl]- N-[(3S)- pyrrolidin-3- yl]piperazine-1- carboxamide formate | 661.1 | 661.2 | In analogy to procedures described example 1/12: Suzuki reaction of [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) with 1-(2-methoxyethyl)-4- (4,4,5,5-tetramethyl-1,3,2- dioxaborolan-2-yl)-3- (trifluoromethyl)pyrazole, acidic Boc deprotection, coupling with (S)-3-amino-1-N-Boc-pyrrolidine and subsequent acidic Boc deprotection |
| 294 | 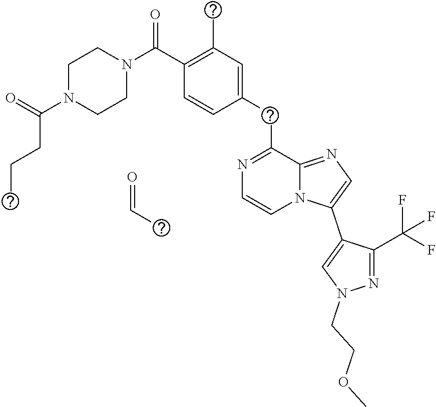 | 3-amino-1-[4-[2- chloro-4-[[3-[1- (2-methoxyethyl)- 3- (trifluoromethyl) pyrazol-4- yl]imidazo[1,2- a]pyrazin-8- yl]amino]benzoyl] piperazin-1- yl]propan-1-one formate | 620.0 | 620.2 | In analogy to procedures described example 1/12: Suzuki reaction of [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) with 1-(2-methoxyethyl)-4- (4,4,5,5-tetramethyl-1,3,2- dioxaborolan-2-yl)-3- (trifluoromethyl)pyrazole, acidic Boc deprotection, coupling with Boc-BETA-ALA-OH and subsequent acidic Boc deprotection |
| 295 | 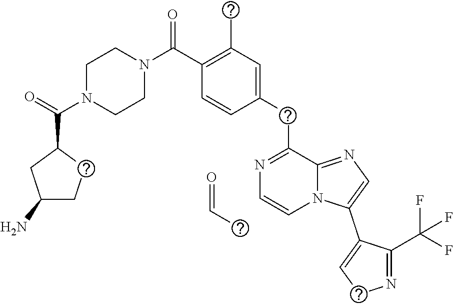 | [4-[(2S,4S)-4- aminopyrrolidine- 2- carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 603.0 | 603.2 | in analogy to the procedures for example 12: amide formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (2S,4S)-1-tert-butoxycarbonyl-4- (tert- butoxycarbonylamino)pyrrolidine- 2-carboxylic acid followed by acidic Boc deprotection |
| 296 | 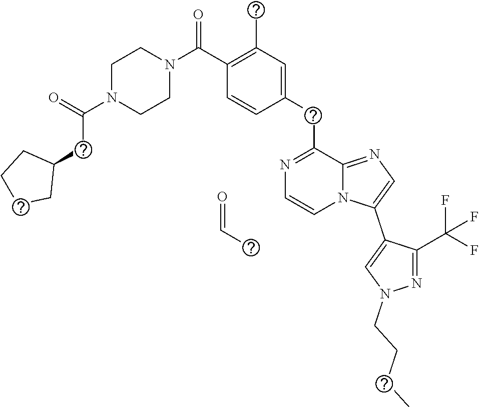 | 4-[2-chloro-4- [[3-[1-(2- methoxyethyl)-3- (trifluoromethyl) pyrazol-4- yl]imidazo[1,2- a]pyrazin-8- yl]amino]benzoyl]- N-[(3R)- pyrrolidin-3- yl]piperazine-1- carboxamide formate | 661.1 | 661.2 | In analogy to procedures described for example 1/12: Suzuki reaction of [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) with 1-(2-methoxyethyl)-4- (4,4,5,5-tetramethyl-1,3,2- dioxaborolan-2-yl)-3- (trifluoromethyl)pyrazole, acidic Boc deprotection, urea formation with (R)-(+)-1-Boc-3- aminopyrrolidine and subsequent acidic Boc deprotection |
| 297 | 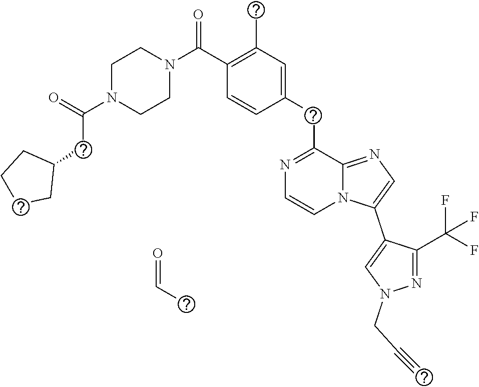 | 4-[2-chloro-4- [[3-[1- (cyanomethyl)-3- (trifluoromethyl) pyrazol-4- yl]imidazo[1,2- a]pyrazin-8- yl]amino]benzoyl]- N-[(3S)- pyrrolidin-3- yl]piperazine-1- carboxamide formate | 642.0 | 642.4 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl (S)-3-aminopyrrolidine- 1-carboxylate, alkylation with 2- bromoacetonitrile followed by acidic Boc deprotection |
| 298 | 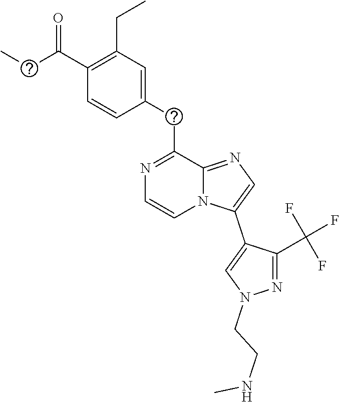 [SMILES: CCC(=O)c1ccc(Cc2nccn3c(-c4cn(CCNC)nc4C(F)(F)F)cnc23)cc1CC] | 2-ethyl-N- methyl-4-[[3-[1- [2- (methylamino) ethyl]-3- (trifluoromethyl) pyrazol-4- yl]imidazo[1,2- a]pyrazin-8- yl]amino]benzamide | 486.5 | 487.5 | in analogy to the procedures for example 15: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with tert-butyl (2- hydroxyethyl)(methyl)carbamate [CAS#57561-39-4] and subsequent acidic Boc deprotection |
| 299 | 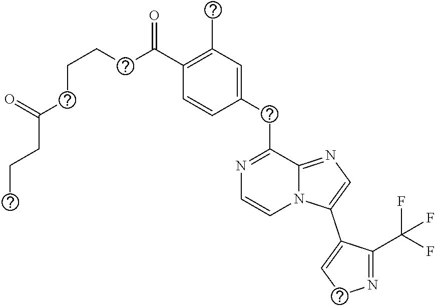 [SMILES: CCCC(=O)CCCCC(=O)c1ccc(Cc2nccn3c(C4=CCN=C4C(F)(F)F)cnc23)cc1C] | N-[2-(3- aminopropanoyl amino)ethyl]-2- chloro-4-[[3-[3- (trifluoromethyl)- 1H-pyrazol-4- yl]imidazo[1,2- a]pyrazin-8- yl]amino]benzamide | 535.9 | 536.1 | in analogy to the procedures for example 1: amide coupling of 2- chloro-4-[(3-iodoimidazo[1,2- a]pyrazin-8-yl)amino]benzoic acid (example 14, step 1) with N-Boc- ethylenediamine, subsequent Suzuki reaction with (3- (trifluoromethyl)-1H-pyrazol-4- yl)boronic acid, acidic Boc deprotection, coupling with Boc- BETA-ALA-OH and subsequent acidic Boc deprotection |
| 300 | 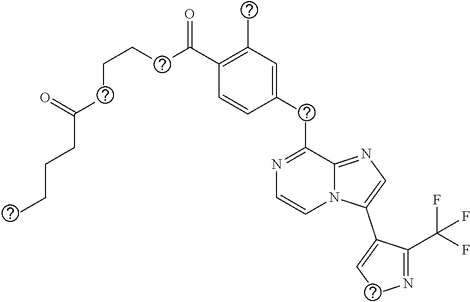 [SMILES: CCCCC(=O)CCCCC(=O)c1ccc(Cc2nccn3c(C4=CCN=C4C(F)(F)F)cnc23)cc1C] | N-[2-(4- aminobutanoyl amino)ethyl]-2- chloro-4-[[3-[3- (trifluoromethyl)- 1H-pyrazol-4- yl]imidazo[1,2- a]pyrazin-8- yl]amino]benzamide | 549.9 | 550.1 | in analogy to the procedures for example 1: amide coupling of 2- chloro-4-[(3-iodoimidazo[1,2- a]pyrazin-8-yl)amino]benzoic acid (example 14, step 1) with N-Boc- ethylenediamine, subsequent Suzuki reaction with (3- (trifluoromethyl)-1H-pyrazol-4- yl)boronic acid, acidic Boc deprotection, coupling with BOC- gamma-abu-OH and subsequent acidic Boc deprotection |
| 301 | 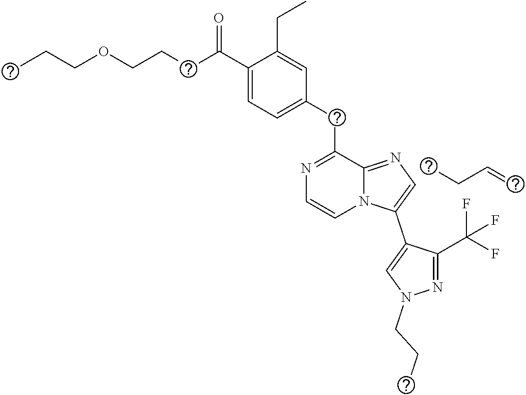 | N-[2-(2- aminoethoxy)ethyl]- 2-ethyl-4-[[3- [1-(2-fluoroethyl)- 3- (trifluoromethyl) pyrazol-4- yl]imidazo[1,2- a]pyrazin-8- yl]amino]benzamide formate | 548.5 | 549.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 1-bromo-2- fluoroethane and subsequent acidic cleavage of the Boc group |
| 302 | 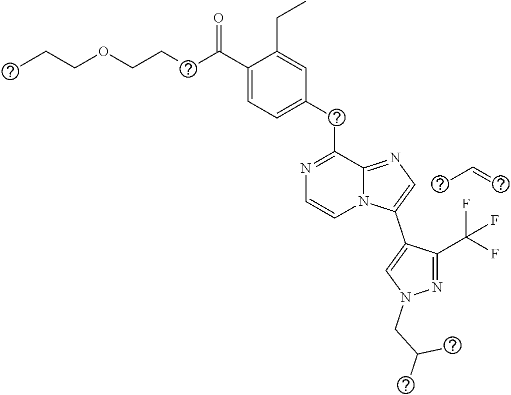 | N-[2-(2- aminoethoxy)ethyl]- 4-[[3-[1-(2,2- difluoroethyl)-3- (trifluoromethyl) pyrazol-4- yl]imidazo[1,2- a]pyrazin-8- yl]amino]-2- ethylbenzamide formate | 566.5 | 567.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 1,1-difluoro-2- iodo-ethane and subsequent acidic cleavage of the Boc group |
| 303 | 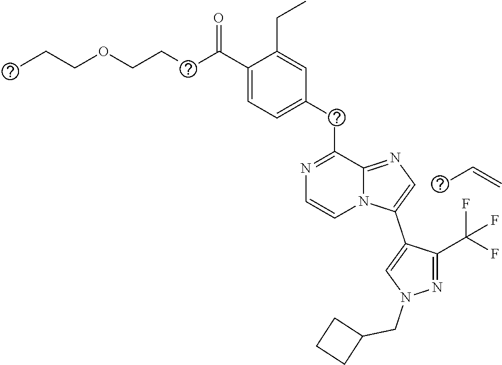 | N-[2-(2- aminoethoxy)ethyl]- 4-[[3-[1- (cyclobutylmethyl)- 3- (trifluoromethyl) pyrazol-4- yl]imidazo[1,2- a]pyrazin-8- yl]amino]-2- ethylbenzamide formate | 570.6 | 571.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with (bromomethyl)cyclobutane and subsequent acidic cleavage of the Boc group |
| 304 | 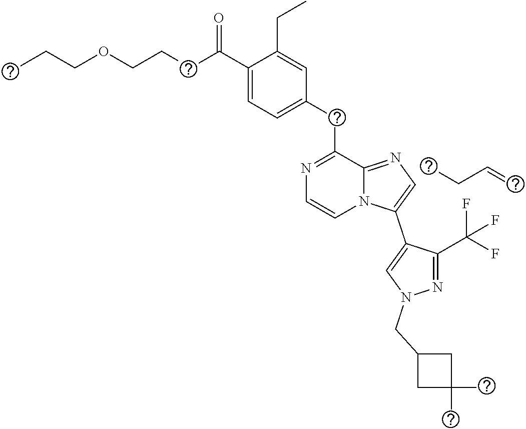 | N-[2-(2- aminoethoxy)ethyl]- 4-[[3-[1-[(3,3- difluorocyclobutyl) methyl]-3- (trifluoromethyl) pyrazol-4- yl]imidazo[1,2- a]pyrazin-8- yl]amino]-2- ethylbenzamide formate | 606.6 | 607.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3-(bromomethyl)- 1,1-difluorocyclobutane and subsequent acidic cleavage of the Boc group |
| 305 | 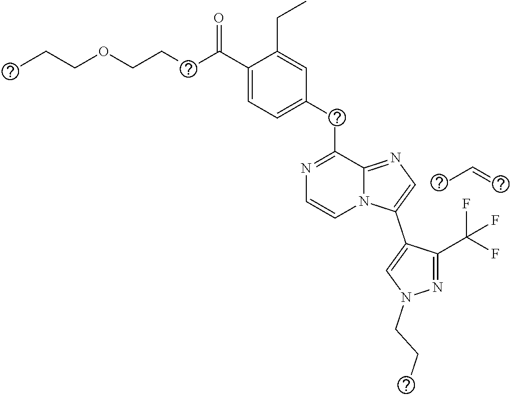 | N-[2-(2- aminoethoxy)ethyl]- 2-ethyl-4-[[3- [1-(2- hydroxyethyl)-3- (trifluoromethyl) pyrazol-4- yl]imidazo[1,2- a]pyrazin-8- yl]amino]benzamide formate | 546.5 | 547.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 2-iodoethan-1-ol and subsequent acidic cleavage of the Boc group |
| 306 | 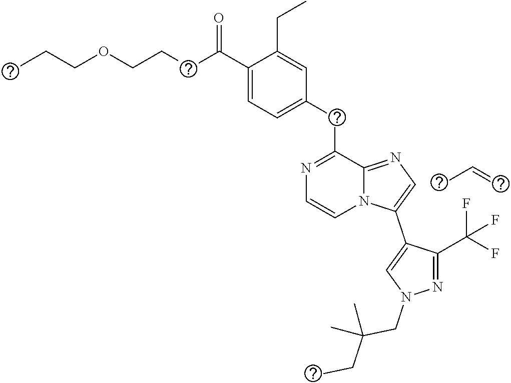 | N-[2-(2- aminoethoxy)ethyl]- 2-ethyl-4-[[3- [1-(3-hydroxy- 2,2- dimethylpropyl)- 3- (trifluoromethyl) pyrazol-4- yl]imidazo[1,2- a]pyrazin-8- yl]amino]benzamide formate | 588.6 | 589.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3-bromo-2,2- dimethylpropan-1-ol and subsequent acidic cleavage of the Boc group |
| 307 | 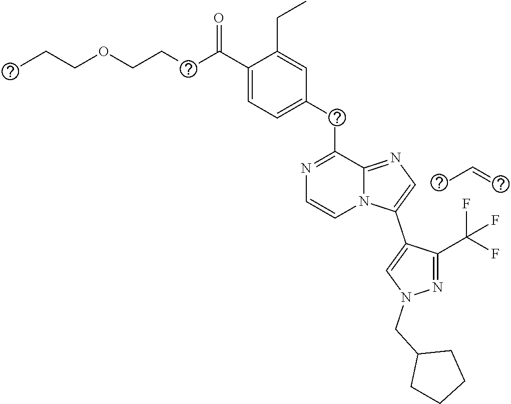 | N-[2-(2- aminoethoxy)ethyl]- 2-ethyl-4-[[3- [1-(oxolan-3- ylmethyl)-3- (trifluoromethyl) pyrazol-4- yl]imidazo[1,2- a]pyrazin-8- yl]amino]benzamide formate | 586.6 | 587.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3- (iodomethyl)tetrahydrofuran and subsequent acidic cleavage of the Boc group |
| 308 | 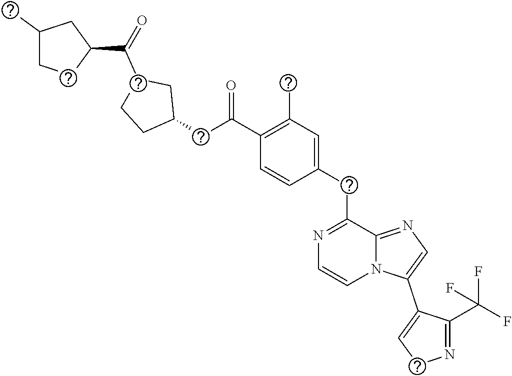 [SMILES: Cc1cc(Cc2nccn3c(C4=CCN=C4C(F)(F)F)cnc23)ccc1C(=O)C[C@@H]1CCC(C(=O)[C@H]2CCC(C)C2)C1] | 2-chloro-N- [(3R)-1-[(2S,4R)- 4- hydroxypyrrolidine- 2- carbonyl]pyrrolidine- 3-yl]-4-[[3-[3- (trifluoromethyl)- 1H-pyrazol-4- yl]imidazo[1,2- a]pyrazin-8- yl]amino]benzamide | 604.0 | 604.3 | in analogy to the procedure for example 6: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl (S)-3-aminopyrrolidine-1- carboxylate, subsequent acidic Boc deprotection, coupling with (2S,4R)-1-(tert-butoxycarbonyl)-4- hydroxypyrrolidine-2-carboxylic acid [13726-69-7] and subsequent acidic Boc deprotection |
| 309 | 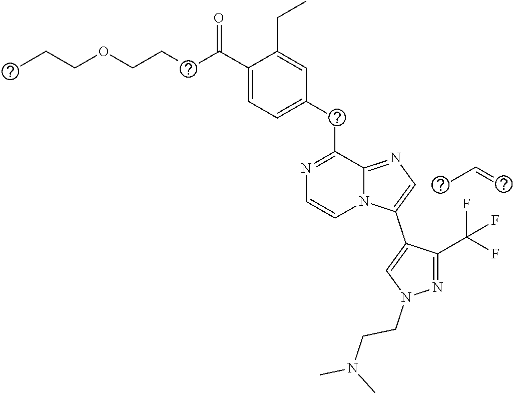 | N-[2-(2- aminoethoxy)ethyl]- 4-[[3-[1-[2- (dimethylamino) ethyl]-3- (trifluoromethyl) pyrazol-4- yl]imidazo[1,2- a]pyrazin-8- yl]amino]-2- ethylbenzamide formate | 573.6 | 574.5 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 2-iodo-N,N- dimethylethan-1-amine and subsequent acidic cleavage of the Boc group |
| 310 | 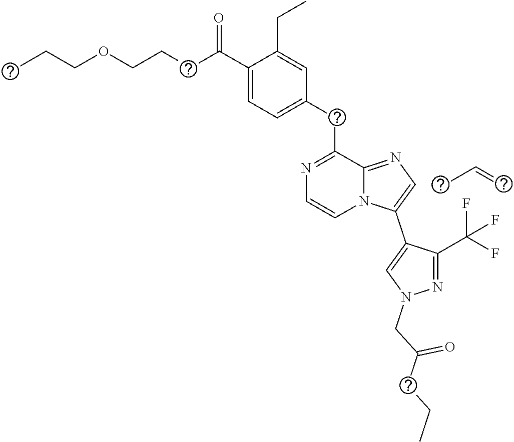 | ethyl 2-[4-[8-[4- [2-(2- aminoethoxy) ethylcarbamoyl]-3- ethylanilino]imidazo [1,2-a]pyrazin- 3-yl]-3- (trifluoromethyl) pyrazol-1- yl]acetate formate | 588.6 | 589.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with ethyl 2-iodoacetate and subsequent acidic cleavage of the Boc group |
| 311 | 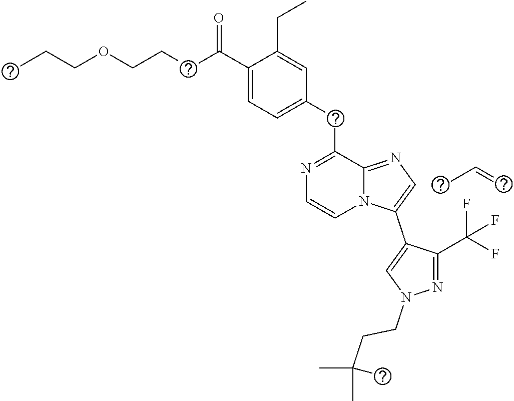 | N-[2-(2- aminoethoxy)ethyl]- 2-ethyl-4-[[3- [1-(3-hydroxy-3- methylbutyl)-3- (trifluoromethyl) pyrazol-4- yl]imidazo[1,2- a]pyrazin-8- yl]amino]benzamide formate | 588.6 | 589.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 4-iodo-2- methylbutan-2-ol and subsequent acidic cleavage of the Boc group |
| 312 | 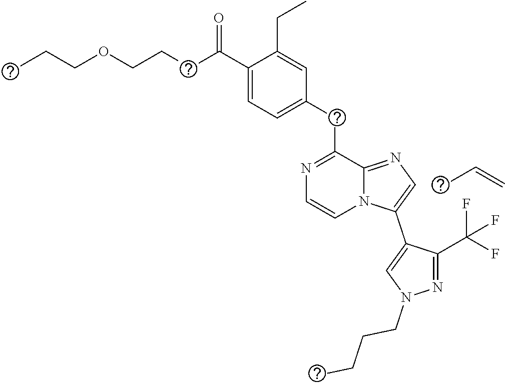 | N-[2-(2- aminoethoxy)ethyl]- 2-ethyl-4-[[3- [1-(3- hydroxypropyl)-3- (trifluoromethyl) pyrazol-4- yl]imidazo[1,2- a]pyrazin-8- yl]amino]benzamide formate | 560.6 | 561.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3-iodopropan-1-ol and subsequent acidic cleavage of the Boc group |
| 313 | 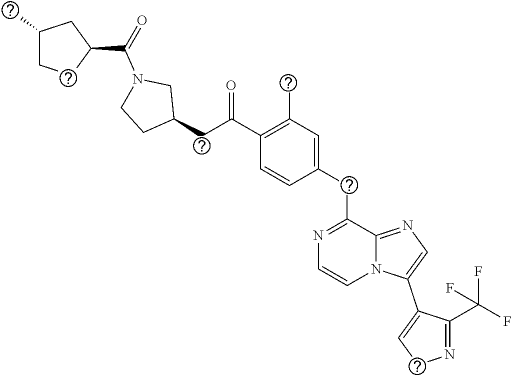 [SMILES: Cc1cc(Cc2nccn3c(C4=CCN=C4C(F)(F)F)cnc23)ccc1C(=O)C[C@H]1CCN(C(=O)[C@H]2CC[C@H](C)C2)C1] | 2-chloro-N-[(3S)- 1-[(2S,4R)-4- hydroxypyrrolidine- 2- carbonyl]pyrrolidin- 3-yl]-4-[[3-[3- (trifluoromethyl)- 1H-pyrazol-4- yl]imidazo[1,2- a]pyrazin-8- yl]amino]benzamide | 604.0 | 604.3 | in analogy to procedures for example 6: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl (R)-3-aminopyrrolidine-1- carboxylate, subsequent acidic Boc deprotection, coupling with (2S,4R)-1-(tert-butoxycarbonyl)-4- hydroxypyrrolidine-2-carboxylic acid [13726-69-7] and subsequent acidic Boc deprotection |
| 314 | 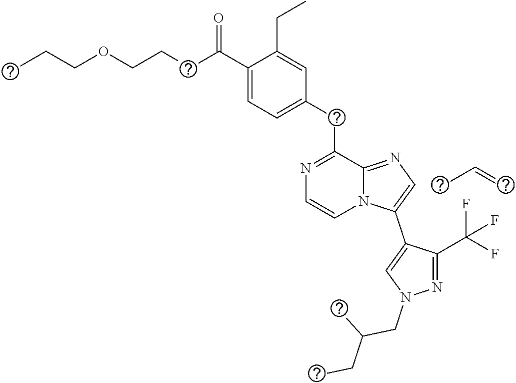 | N-[2-(2- aminoethoxy)ethyl]- 4-[[3-[1-(2,3- dihydroxypropyl)- 3- (trifluoromethyl) pyrazol-4- yl]imidazo[1,2- a]pyrazin-8- yl]amino]-2- ethylbenzamide formate | 576.6 | 577.4 | in analogy to the procedures for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 3-bromopropane- 1,2-diol and subsequent acidic cleavage of the Boc group |
| 315 | 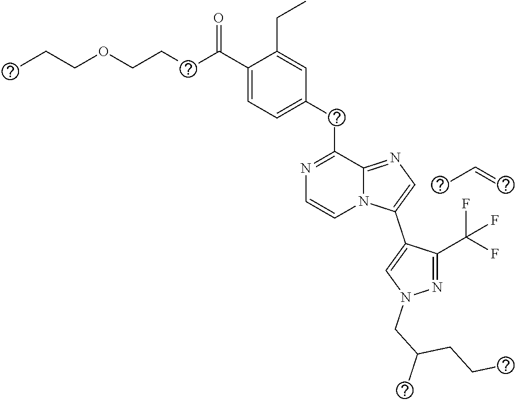 | N-[2-(2- aminoethoxy)ethyl]- 4-[[3-[1-(2,4- dihydroxybutyl)- 3- (trifluoromethyl) pyrazol-4- yl]imidazo[1,2- a]pyrazin-8- yl]amino]-2- ethylbenzamide formate | 590.6 | 591.4 | in analogy to procedures for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 2- (bromomethyl)oxetane and subsequent acidic cleavage of the Boc group |
| 316 | 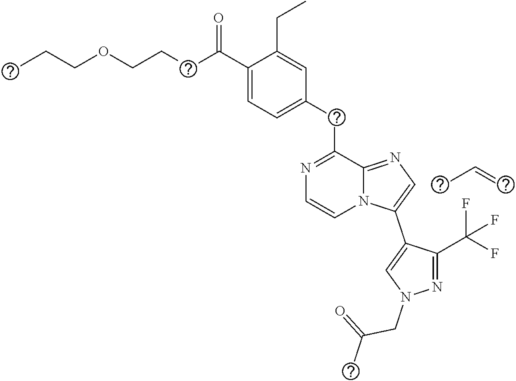 | N-[2-(2- aminoethoxy)ethyl]- 4-[[3-[1-(2- amino-2- oxoethyl)-3- (trifluoromethyl) pyrazol-4- yl]imidazo[1,2- a]pyrazin-8- yl]amino]-2- ethylbenzamide formate | 559.5 | 560.4 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 2-iodoacetamide and subsequent acidic cleavage of the Boc group |
| 317 | 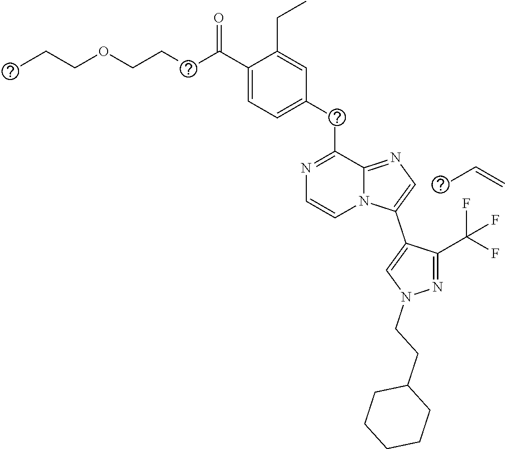 | N-[2-(2- aminoethoxy)ethyl]- 2-ethyl-4-[[3- [1-(2-morpholin- 4-ylethyl)-3- (trifluoromethyl) pyrazol-4- yl]imidazo[1,2- a]pyrazin-8- yl]amino]benzamide formate | 615.7 | 616.5 | in analogy to the procedure for example 2: from tert-butyl N-[2- [2-[[2-ethyl-4-[[3-[3- (trifluoromethyl)-1H-pyrazol-4- yl]imidazo[1,2-a]pyrazin-8- yl]amino]benzoyl]amino]ethoxy] ethyl]carbamate (example 2, step 1), alkylation with 4-(2- iodoethyl)morpholine and subsequent acidic cleavage of the Boc group |
| 318 | 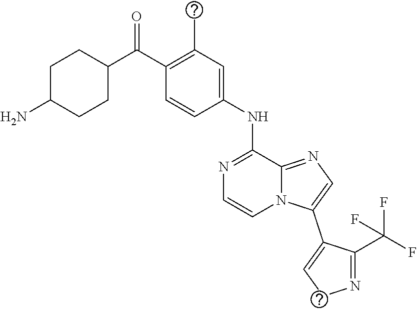 [SMILES: Cc1cc(Nc2nccn3c(C4=CCN=C4C(F)(F)F)cnc23)ccc1C(=O)C1CCC(N)CC1] | (4- aminopiperidin-1- yl)-[2-chloro-4- [[3-[3- (trifluoromethyl)- 1H-pyrazol-4- yl]imidazo[1,2- a]pyrazin-8- yl]amino]phenyl] methanone | 504.9 | 505.3 | in analogy to procedures for example 6: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl piperidin-4-ylcarbamate and subsequent acidic Boc deprotection |
| 319 | 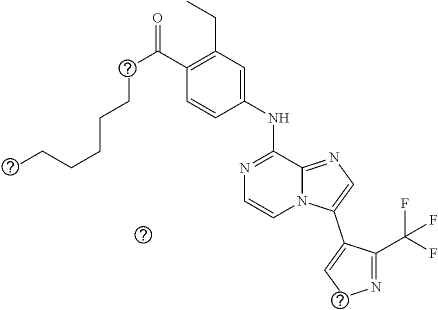 [SMILES: CCCCCCCC(=O)c1ccc(Nc2nccn3c(C4=CCN=C4C(F)(F)F)cnc23)cc1CC] | N-(5- aminopentyl)-2- ethyl-4-[[3-[3- (trifluoromethyl)- 1H-pyrazol-4- yl]imidazo[1,2- a]pyrazin-8- yl]amino]benzamide hydrochloride | 500.5 | 501.5 | in analogy to procedures for example 3: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid (example 3, step 2) with tert-butyl (5- aminopentyl)carbamate and subsequent acidic Boc deprotection |
| 320 | 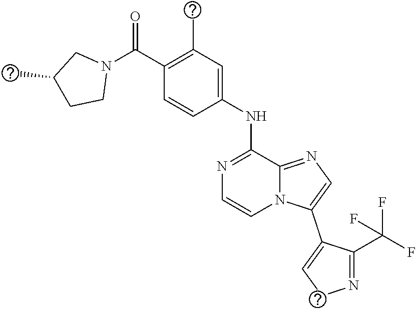 [SMILES: Cc1cc(Nc2nccn3c(C4=CCN=C4C(F)(F)F)cnc23)ccc1C(=O)N1CC[C@H](C)C1] | [(3S)-3- aminopyrrolidin- 1-yl]-[2-chloro-4- [[3-[3- (trifluoromethyl)- 1H-pyrazol-4- yl]imidazo[1,2- a]pyrazin-8- yl]amino]phenyl] methanone | 490.9 | 491.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (S)-pyrrolidin-3-ylcarbamate and subsequent acidic Boc deprotection |
| 321 | 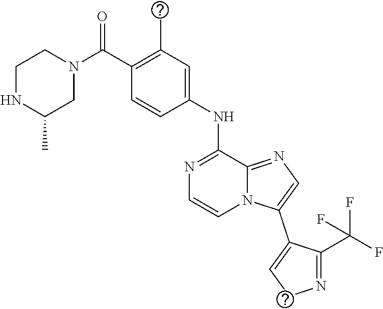 [SMILES: Cc1cc(Nc2nccn3c(C4=CCN=C4C(F)(F)F)cnc23)ccc1C(=O)N1CCN[C@@H](C)C1] | [2-chloro-4-[[3- [3- (trifluoromethyl)- 1H-pyrazol-4- yl]imidazo[1,2- a]pyrazin-8- yl]amino]phenyl]- [(3S)-3- methylpiperazin- 1-yl]methanone | 504.9 | 505.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (S)-2-methylpiperazine-1- carboxylate and subsequent acidic Boc deprotection |
| 322 | 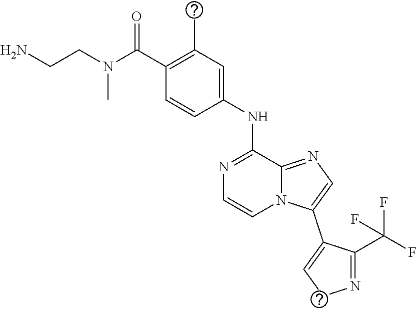 [SMILES: Cc1cc(Nc2nccn3c(C4=CCN=C4C(F)(F)F)cnc23)ccc1C(=O)N(C)CCN] | N-(2- aminoethyl)-2- chloro-N-methyl- 4-[[3-[3- (trifluoromethyl)- 1H-pyrazol-4- yl]imidazo[1,2- a]pyrazin-8- yl]amino]benzamide | 478.9 | 479.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (2-(methylamino)ethyl)carbamate and subsequent acidic Boc deprotection |
| 323 | 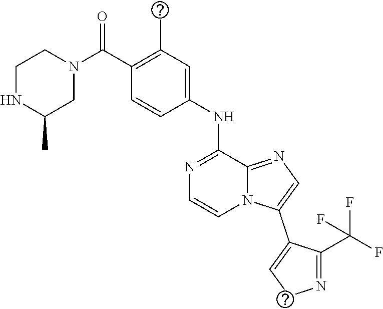 [SMILES: Cc1cc(Nc2nccn3c(C4=CCN=C4C(F)(F)F)cnc23)ccc1C(=O)N1CCN[C@H](C)C1] | [2-chloro-4-[[3- [3- (trifluoromethyl)- 1H-pyrazol-4- yl]imidazo[1,2- a]pyrazin-8- yl]amino]phenyl]- [(3R)-3- methylpiperazin- 1-yl]methanone | 504.9 | 505.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (R)-2-methylpiperazine-1- carboxylate and subsequent acidic Boc deprotection |
| 324 | 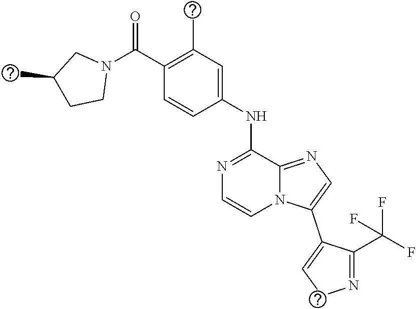 [SMILES: Cc1cc(Nc2nccn3c(C4=CCN=C4C(F)(F)F)cnc23)ccc1C(=O)N1CC[C@@H](C)C1] | [(3R)-3- aminopyrrolidin- 1-yl]-[2-chloro-4- [[3-[3- (trifluoromethyl)- 1H-pyrazol-4- yl]imidazo[1,2- a]pyrazin-8- yl]amino]phenyl] methanone | 490.9 | 491.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (R)-pyrrolidin-3-ylcarbamate and subsequent acidic Boc deprotection |
| 325 | 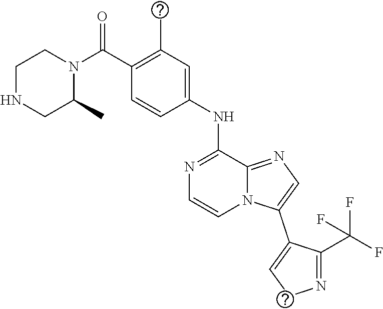 [SMILES: Cc1cc(Nc2nccn3c(C4=CCN=C4C(F)(F)F)cnc23)ccc1C(=O)N1CCNC[C@@H]1C] | [2-chloro-4-[[3- [3- (trifluoromethyl)- 1H-pyrazol-4- yl]imidazo[1,2- a]pyrazin-8- yl]amino]phenyl]- [(2S)-2- methylpiperazin- 1-yl]methanone | 504.9 | 505.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (S)-2-methylpiperazine-1- carboxylate and subsequent acidic Boc deprotection |
| 326 | 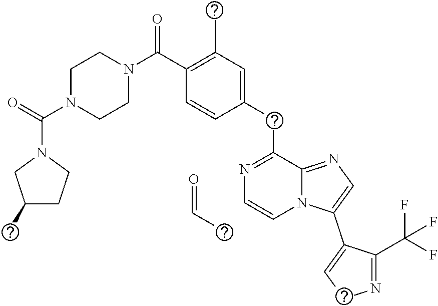 | [4-[(3R)-3- aminopyrrolidine- 1- carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 603.0 | 603.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (R)-3-(Boc-amino)pyrrolidine followed by acidic Boc deprotection |
| 327 | 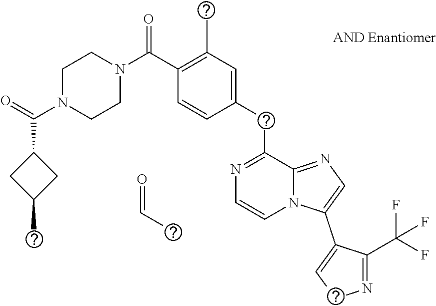 | [4-(3- aminocyclobutane carbonyl)piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 588.0 | 588.2 | in analogy to the procedures for example 6: amide coupling of [2- chloro-4-[[3-[3-(trifluoromethyl)- 1H-pyrazol-4-yl]imidazo[1,2- a]pyrazin-4-yl]imidazo[1,2- a]pyrazin-8-yl-methanone (example 6, step 5) with 3-(tert- butoxycarbonylamino)cyclobutane carboxylic acid and acidic Boc deprotection |
| 328 | 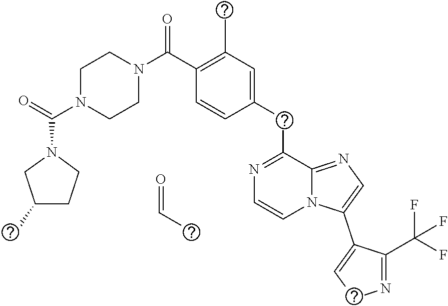 | [4-[(3S)-3- aminopyrrolidine- 1- carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 603.0 | 603.1 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (S)-3-(Boc-amino)pyrrolidine followed by acidic Boc deprotection |
| 329 | 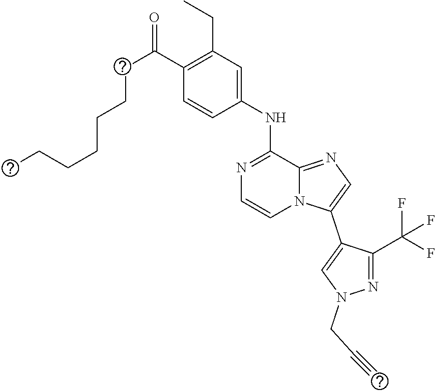 [SMILES: C#CCn1cc(-c2cnc3c(Nc4ccc(C(=O)CCCCCCC)c(CC)c4)nccn23)c(C(F)(F)F)n1] | N-(5- aminopentyl)-4- [[3-[1- (cyanomethyl)-3- (trifluoromethyl) pyrazol-4- yl]imidazo[1,2- a]pyrazin-8- yl]amino]-2- ethylbenzamide | 539.6 | 540.3 | in analogy to the procedures for example 3: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid (example 3, step 2) with tert-butyl (5- aminopentyl)carbamate, alkylation with 2-bromoacetonitrile and subsequent acidic Boc deprotection |
| 330 | 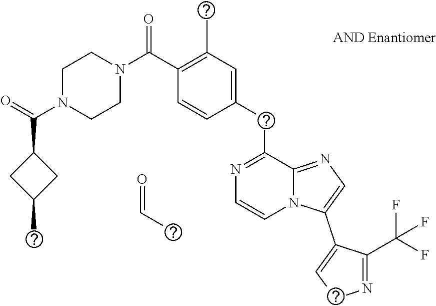 | [4-(3- aminocyclobutane carbonyl)piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 588.0 | 588.2 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 3-(tert- butoxycarbonylamino)cyclobutane carboxylic acid and acidic Boc deprotection |
| 331 | 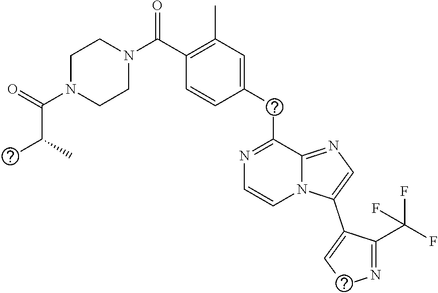 | (2S)-2-amino-1- [4-[2-methyl-4- [[3-[3- (trifluoromethyl)- 1H-pyrazol-4- yl]imidazo[1,2- a]pyrazin-8- yl]amino]benzoyl] piperazin-1- yl]propan-1-one | 541.5 | 542.4 | in analogy to the procedures for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (tert- butoxycarbonyl)-L-alanine and acidic Boc deprotection |
| 332 | 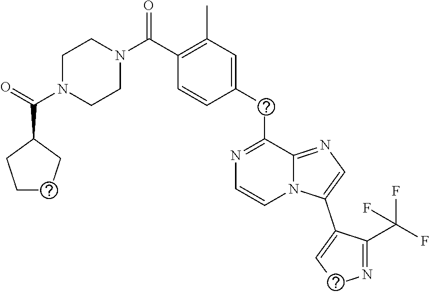 | [2-methyl-4-[[3- [3- (trifluoromethyl)- 1H-pyrazol-4- yl]imidazo[1,2- a]pyrazin-8- yl]amino]phenyl]- [4-[(3R)- pyrrolidine-3- carbonyl]piperazin- 1-yl]methanone | 567.6 | 568.4 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (R)-1- (tert-butoxycarbonyl)pyrrolidine- 3-carboxylic acid and acidic Boc deprotection |
| 333 | 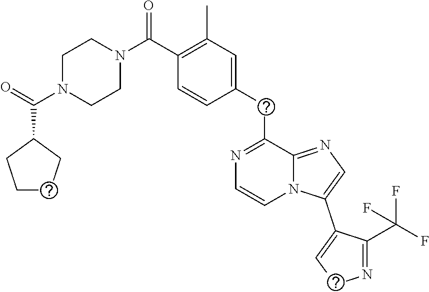 | [2-methyl-4-[[3- [3- (trifluoromethyl)- 1H-pyrazol-4- yl]imidazo[1,2- a]pyrazin-8- yl]amino]phenyl]- [4-[(3S)- pyrrolidine-3- carbonyl]piperazin- 1-yl]methanone | 567.6 | 568.4 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (S)-1- (tert-butoxycarbonyl)pyrrolidine- 3-carboxylic acid and acidic Boc deprotection |
| 334 | 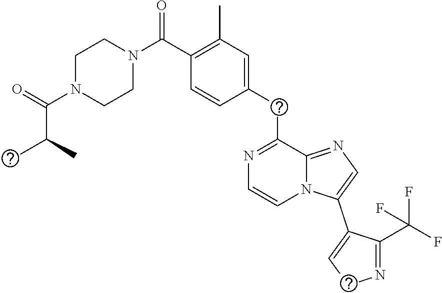 | (2R)-2-amino-1- [4-[2-methyl-4- [[3-[3- (trifluoromethyl)- 1H-pyrazol-4- yl]imidazo[1,2- a]pyrazin-8- yl]amino]benzoyl] piperazin-1- yl]propan-1-one | 541.5 | 542.4 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (tert- butoxycarbonyl)-D-alanine and acidic Boc deprotection |
| 335 | 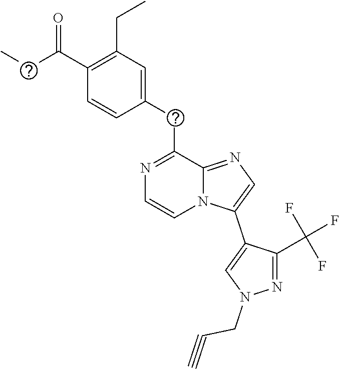 [SMILES: C#CCn1cc(-c2cnc3c(Cc4ccc(C(=O)CC)c(CC)c4)nccn23)c(C(F)(F)F)n1] | 2-ethyl-N- methyl-4-[[3-[1- prop-2-ynyl-3- (trifluoromethyl) pyrazol-4- yl]imidazo[1,2- a]pyrazin-8- yl]amino]benzamide | 467.4 | 468.4 | in analogy to the procedures for example 2: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with 3-bromoprop-1-yne |
| 336 | 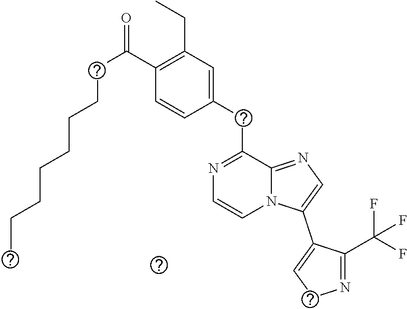 [SMILES: CCCCCCCCC(=O)c1ccc(Cc2nccn3c(C4=CCN=C4C(F)(F)F)cnc23)cc1CC] | N-(6- aminohexyl)-2- ethyl-4-[[3-[3- (trifluoromethyl)- 1H-pyrazol-4- yl]imidazo[1,2- a]pyrazin-8- yl]amino]benzamide hydrochloride | 514.5 | 515.5 | in analogy to procedures for example 3: amide coupling of 2- ethyl-4-[[3-[3-(trifluoromethyl)- 1H-pyrazol-4-yl]imidazo[1,2- a]pyrazin-8-yl]amino]benzoic acid, hydrochloric acid (example 3, step 2) with tert-butyl (6- aminohexyl)carbamate hydrochloride and subsequent acidic Boc deprotection |
| 337 | 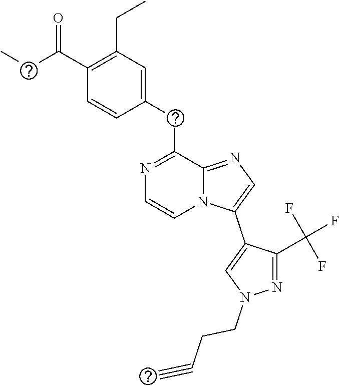 [SMILES: C#CCCn1cc(-c2cnc3c(Cc4ccc(C(=O)CC)c(CC)c4)nccn23)c(C(F)(F)F)n1] | 4-[[3-[1-(2- cyanomethyl)-3- (trifluoromethyl) pyrazol-4- yl]imidazo[1,2- a]pyrazin-8- yl]amino]-2-ethyl- N- methylbenzamide | 482.5 | 483.4 | in analogy to the procedures for example 2: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with 3-bromopropanenitrile [CAS#2417-90-5] |
| 338 | 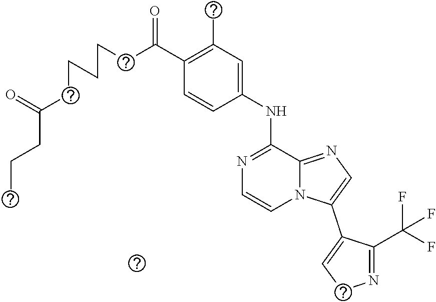 [SMILES: CCCC(=O)CCCCCC(=O)c1ccc(Nc2nccn3c(C4=CCN=C4C(F)(F)F)cnc23)cc1C] | N-[3-(3- aminopropanoyl amino)propyl]-2- chloro-4-[[3-[3- (trifluoromethyl)- 1H-pyrazol-4- yl]imidazo[1,2- a]pyrazin-8- yl]amino]benzamide hydrochloride | 549.9 | 550.2, | in analogy to the procedures for example 1: amide coupling of 2- chloro-4-[(3-iodoimidazo[1,2- a]pyrazin-8-yl)amino]benzoic acid (example 14, step 1) with N-Boc- 1,3-diaminopropane, subsequent Suzuki reaction with [3- (trifluoromethyl)-1-trityl-pyrazol- 4-yl]methanediol, acidic Boc deprotection, coupling with Boc- BETA-ALA-OH and subsequent acidic Boc deprotection |
| 339 | 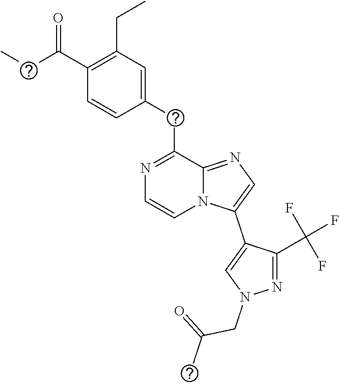 [SMILES: CCC(=O)c1ccc(Cc2nccn3c(-c4cn(CC(C)=O)nc4C(F)(F)F)cnc23)cc1CC] | 4-[[3-[1-(2- amino-2- oxoethyl)-3- (trifluoromethyl) pyrazol-4- yl]imidazo[1,2- a]pyrazin-8- yl]amino]-2-ethyl- N- methylbenzamide | 486.5 | 487.4 | in analogy to the procedures for example 2: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with 2-chloroacetamide [CAS#79-07-2] |
| 340 | 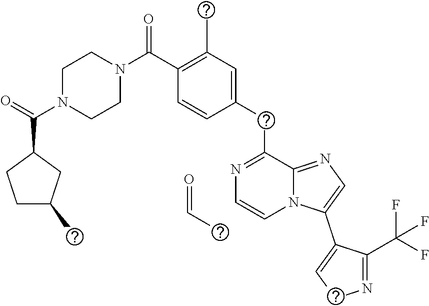 | [4-[(1R,3S)-3- aminocyclopentane carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 602.0 | 602.2 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (−)- (1R,3S)-N-Boc-3- aminocyclopentanecarboxylic acid and acidic Boc deprotection |
| 341 | 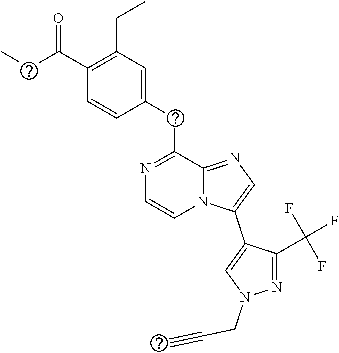 [SMILES: C#CCn1cc(-c2cnc3c(Cc4ccc(C(=O)CC)c(CC)c4)nccn23)c(C(F)(F)F)n1] | 4-[[3-[1- (cyanomethyl)-3- (trifluoromethyl) pyrazol-4- yl]imidazo[1,2- a]pyrazin-8- yl]amino]-2-ethyl- N- methylbenzamide | 468.4 | 469.3 | in analogy to the procedures for example 2: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with 2-bromoacetonitrile |
| 342 | 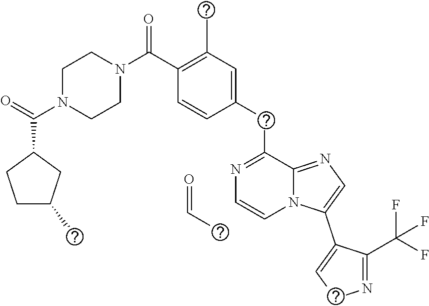 | [4-[(1S,3R)-3- aminocyclopentane carbonyl]piperazin- 1-yl]-[2-chloro- 4-[[3-[3- (trifluoromethyl)- 1H-pyrazol-4- yl]imidazo[1,2- a]pyrazin-8- yl]amino]phenyl] methanone formate | 602.0 | 602.1 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (1R,3R)- N-Boc-3-amino cyclopentanecarboxylic acid and acidic Boc deprotection |
| 343 | 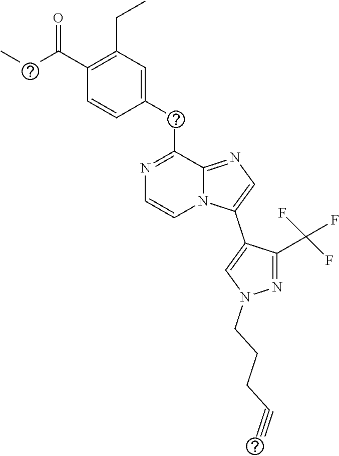 [SMILES: C#CCCCn1cc(-c2cnc3c(Cc4ccc(C(=O)CC)c(CC)c4)nccn23)c(C(F)(F)F)n1] | 4-[[3-[1-(3- cyanopropyl)-3- (trifluoromethyl) pyrazol-4- yl]imidazo[1,2- a]pyrazin-8- yl]amino]-2-ethyl- N- methylbenzamide | 496.5 | 497.4 | in analogy to the procedures for example 2: alkylation of 2-ethyl- N-methyl-4-[[3-[3- (trifluoromethyl)-1H-pyrazol-4- yl]imidazo[1,2-a]pyrazin-8- yl]amino]benzamide (example 15, step 2) with 4-bromobutanenitrile [CAS#5332-06-9] |
| 344 | 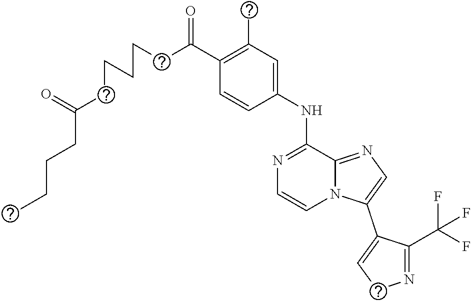 [SMILES: CCCCC(=O)CCCCCC(=O)c1ccc(Nc2nccn3c(C4=CCN=C4C(F)(F)F)cnc23)cc1C] | N-[3-(4- aminobutanoylamino) propyl]-2- chloro-4-[[3-[3- (trifluoromethyl)- 1H-pyrazol-4- yl]imidazo[1,2- a]pyrazin-8- yl]amino]benzamide formate | 564.0 | 564.2 | in analogy to the procedures for example 1: amide coupling of 2- chloro-4-[(3-iodoimidazo[1,2- a]pyrazin-8-yl)amino]benzoic acid (example 14, step 1) with N-Boc- 1,3-diaminopropane, subsequent Suzuki reaction with [3- (trifluoromethyl)-1-trityl-pyrazol- 4-yl]methandediol, acidic Boc deprotection, coupling with Boc- gamm-abu-OH and subsequent acidic Boc deprotection |
| 345 | 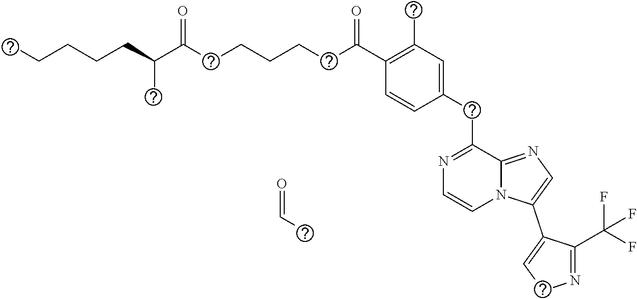 | 2-chloro-N-[3- [[(2S)-2,6- diaminohexanoyl] amino]propyl]-4- [[3-[3- (trifluoromethyl)- 1H-pyrazol-4- yl]imidazo[1,2- a]pyrazin-8- yl]amino]benzamide foramate | 607.0 | 607.3 | in analogy to the procedures for example 1: amide coupling of 2- chloro-4-[(3-iodoimidazo[1,2- a]pyrazin-8-yl)amino]benzoic acid (example 14, step 1) with N-Boc- 1,3-diaminopropane, subsequent Suzuki reaction with [3- (trifluoromethyl)-1-trityl-pyrazol- 4-yl]methanediol, acidic Boc deprotection, coupling with Boc- LYS(Boc)-OH and subsequent acidic Boc deprotection |
| 346 | 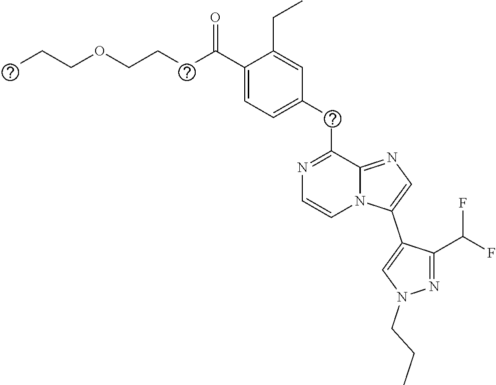 [SMILES: CCCOCCCC(=O)c1ccc(Cc2nccn3c(-c4cn(CCC)nc4C(F)F)cnc23)cc1CC] | N-[2-(2- aminoethoxy)ethyl]- 4-[[3-[3- (difluoromethyl)- 1-propylpyrazol- 4-yl]imidazo[1,2- a]pyrazin-8- yl]amino]-2- ethylbenzamide | 526.6 | 527.4 | in analogy to the procedures for example 2: alkylation of tert-butyl N-[2-[2-[[4-[[3-[3- (difluoromethyl)-1H-pyrazol-4- yl]imidazo[1,2-a]pyrazin-8- yl]amino]-2-ethyl- benzoyl]amino]ethoxy]ethyl] carbamate (example 18, step 1) with 1- iodopropane and subsequent acidic Boc deprotection |
| 347 | 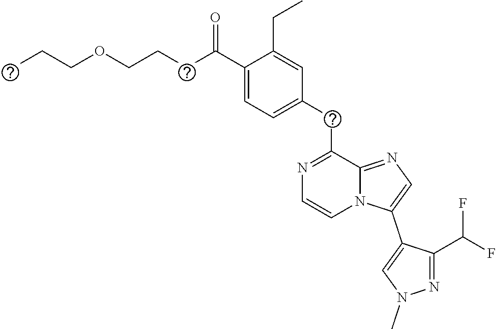 [SMILES: CCCOCCCC(=O)c1ccc(Cc2nccn3c(-c4cn(C)nc4C(F)F)cnc23)cc1CC] | N-[2-(2- aminoethoxy)ethyl]- 4-[[3-[3- (difluoromethyl)- 1-methylpyrazol- 4-yl]imidazo[1,2- a]pyrazin-8- yl]amino]-2- ethylbenzamide | 498.5 | 499.4 | in analogy to the procedures for example 2: alkylation of tert-butyl N-[2-[2-[[4-[[3-[3- (difluoromethyl)-1H-pyrazol-4- yl]imidazo[1,2-a]pyrazin-8- yl]amino]-2-ethyl- benzoyl]amino]ethoxy]ethyl] carbamate (example 18, step 1) with iodomethane and subsequent acidic Boc deprotection |
| 348 | 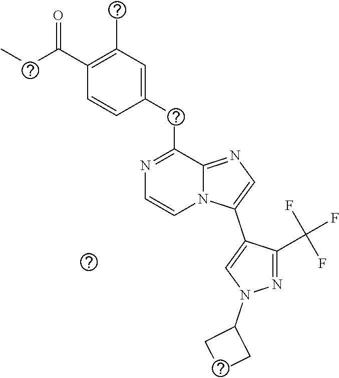 [SMILES: CCC(=O)c1ccc(Cc2nccn3c(-c4cn(C5CCC5)nc4C(F)(F)F)cnc23)cc1C] | 4-[[3-[1- (azetidin-3-yl)-3- (trifluoromethyl) pyrazol-4- yl]imidazo[1,2- a]pyrazin-8- yl]amino]-2- chloro-N- methylbenzamide hydrochloride | 490.9 | 489.4 [M − H]− | in analogy to the procedures for example 2: alkylation of 2-chloro- N-methyl-4-[[3-[3- (trifluoromethyl)-1H-pyrazol-4- yl]imidazo[1,2-a]pyrazin-8- yl]amino]benzamide (example 10, step 2) with tert-butyl 3- bromoazetidine-1-carboxylate [CAS#1064194-10-0] and subsequent acidic Boc deprotection |
| 349 | 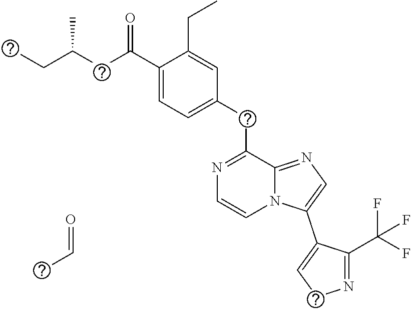 | N-[(2S)-1- aminopropan-2- yl]-2-ethyl-4-[[3- [3- (trifluoromethyl)- 1H-pyrazol-4- yl]imidazo[1,2- a]pyrazin-8- yl]amino]benzamide formate | 472.5 | 473.3 | in analogy to the procedures for example 1: amide coupling of 2- ethyl-4-[(3-iodoimidazo[1,2- a]pyrazin-8-yl)amino]benzoic acid, hydrochloride with tert-butyl (S)- (2-aminopropyl)carbamate, subsequent Suzuki reaction with (3-(trifluoromethyl)-1H-pyrazol-4- yl)boronic acid and acidic Boc deprotection |
| 350 | 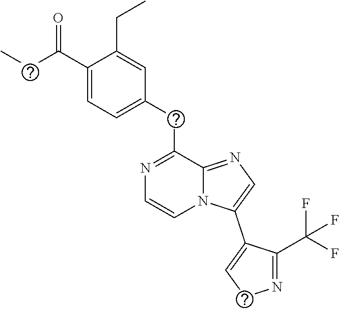 [SMILES: CCC(=O)c1ccc(Cc2nccn3c(C4=CCN=C4C(F)(F)F)cnc23)cc1CC] | 2-ethyl-N- methyl-4-[[3-[3- (trifluoromethyl)- 1H-pyrazol-4- yl]imidazo[1,2- a]pyrazin-8- yl]amino]benzamide | 429.4 | 430.4 | example 15, step 2 |
| 351 | 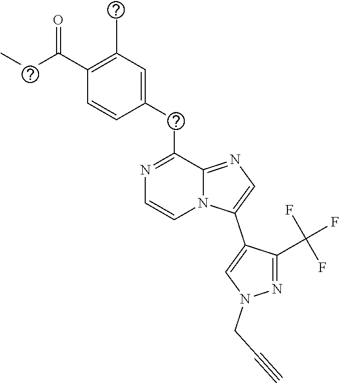 [SMILES: C#CCn1cc(-c2cnc3c(Cc4ccc(C(=O)CC)c(C)c4)nccn23)c(C(F)(F)F)n1] | 2-chloro-N- methyl-4-[[3-[1- prop-2-ynyl-3- (trifluoromethyl) pyrazol-4- yl]imidazo[1,2- a]pyrazin-8- yl]amino]benzamide | 473.8 | 474.3 | In analogy to the procedure described for example 2: alkylation of 2-chloro-N-methyl-4- [[3-[3-(trifluoromethyl)-1H- pyrazol-4-yl]imidazo[1,2- a]pyrazin-8-yl]amino]benzamide (example 10, step 3) and 3- bromoprop-1-yne |
| 352 | 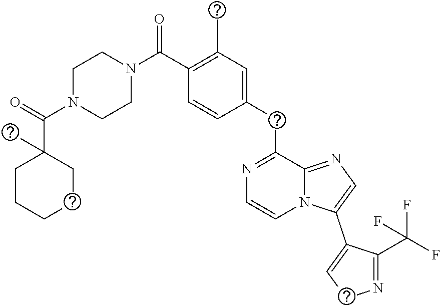 [SMILES: Cc1cc(Cc2nccn3c(C4=CCN=C4C(F)(F)F)cnc23)ccc1C(=O)N1CCN(C(=O)C2(C)CCCCC2)CC1] | [2-chloro-4-[[3- [3- (trifluoromethyl)- 1H-pyrazol-4- yl]imidazo[1,2- a]pyrazin-8- yl]amino]phenyl]- [4-(3- hydroxypiperidine- 3- carbonyl)piperazin- 1-yl]methanone | 618.0 | 618.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 1-(tert- butoxycarbonyl)-3- hydroxypiperidine-3-carboxylic acid CAS [1396555-79-5] and acidic Boc deprotection |
| 353 | 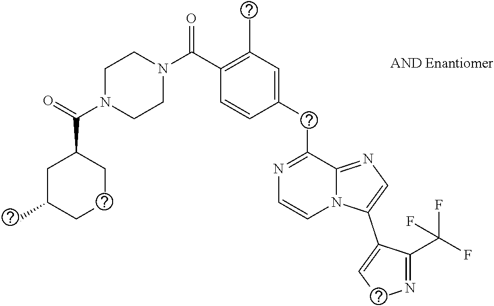 [SMILES: Cc1cc(Cc2nccn3c(C4=CCN=C4C(F)(F)F)cnc23)ccc1C(=O)N1CCN(C(=O)[C@H]2CCC[C@H](C)C2)CC1] | [2-chloro-4-[[3- [3- (trifluoromethyl)- 1H-pyrazol-4- yl]imidazo[1,2- a]pyrazin-8- yl]amino]phenyl]- [4-[rac-(3R,5R)- 5- hydroxypiperidine- 3- carbonyl]piperazin- 1-yl]methanone | 618.0 | 618.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 1-(tert- butoxycarbonyl)-5- hydroxypiperidine-3-carboxylic acid CAS [1095010-48-2] and acidic Boc deprotection |
| 354 | 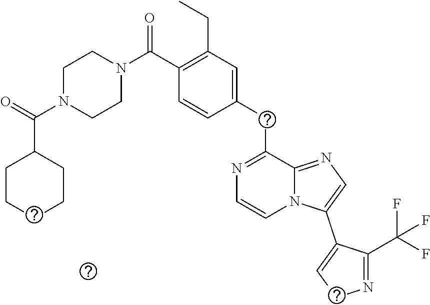 [SMILES: CCc1cc(Cc2nccn3c(C4=CCN=C4C(F)(F)F)cnc23)ccc1C(=O)N1CCN(C(=O)C2CCCCC2)CC1] | [2-ethyl-4-[[3-[3- (trifluoromethyl)- 1H-pyrazol-4- yl]imidazo[1,2- a]pyrazin-8- yl]amino]phenyl]- [4-(piperidine-4- carbonyl)piperazin- 1-yl]methanone hydrochloride | 595.6 | 596.5 | in analogy to procedures described for example 1: through acidic Boc deprotection of tert-butyl 4-[4-[2- ethyl-4-[[3-[3-(trifluoromethyl)- 1H-pyrazol-4-yl]imidazo[1,2- a]pyrazin-8- yl]amino]benzoyl]piperazine-1- carbonyl]piperidine-1-carboxylate (example 5, step 4) |
| 355 | 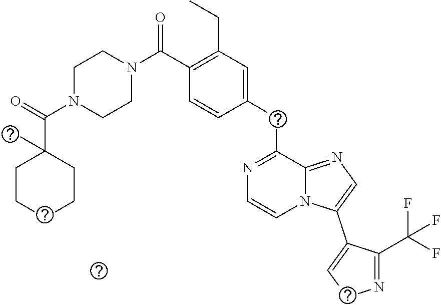 [SMILES: CCc1cc(Cc2nccn3c(C4=CCN=C4C(F)(F)F)cnc23)ccc1C(=O)N1CCN(C(=O)C2(C)CCCCC2)CC1] | [2-ethyl-4-[[3-[3- (trifluoromethyl)- 1H-pyrazol-4- yl]imidazo[1,2- a]pyrazin-8- yl]amino]phenyl]- [4-(4- hydroxypiperidine- 4- carbonyl)piperazin- 1-yl]methanone hydrochloride | 611.6 | 612.6 | in analogy to procedures described for example 1: through acidic Boc deprotection of tert-butyl 4-[4-[2- ethyl-4-[[3-[3-(trifluoromethyl)- 1H-pyrazol-4-yl]imidazo[1,2- a]pyrazin-8- yl]amino]benzoyl]piperazine-1- carbonyl]piperidine-1-carboxylate (example 5, step 4) |
| 356 | 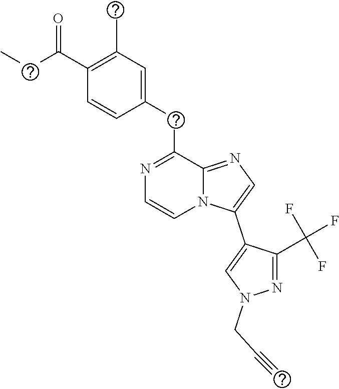 [SMILES: C#CCn1cc(-c2cnc3c(Cc4ccc(C(=O)CC)c(C)c4)nccn23)c(C(F)(F)F)n1] | 2-chloro-4-[[3- [1-(cyanomethyl)- 3- (trifluoromethyl) pyrazol-4- yl]imidazo[1,2- a]pyrazin-8- yl]amino]-2- methylbenzamide | 474.8 | 475.3 | In analogy to the procedure described for example 2: alkylation of 2-chloro-N-methyl-4- [[3-[3-(trifluoromethyl)-1H- pyrazol-4-yl]imidazo[1,2- a]pyrazin-8-yl]amino]benzamide (example 10, step 3) with 2- bromoacetonitrile |
| 357 | 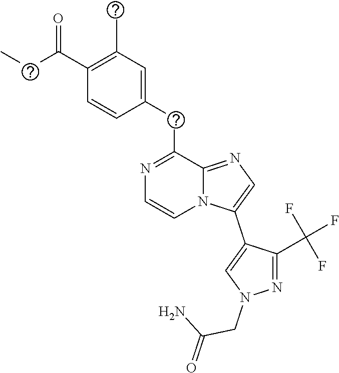 [SMILES: CCC(=O)c1ccc(Cc2nccn3c(-c4cn(CC(N)=O)nc4C(F)(F)F)cnc23)cc1C] | 4-[[3-[1-(2- amino-2- oxoethyl)-3- (trifluoromethyl) pyrazol-4- yl]imidazo[1,2- a]pyrazin-8- yl]amino]-2- chloro-N- methylbenzamide | 492.8 | 493.2 | In analogy to the procedure described for example 2: alkylation of 2-chloro-N-methyl-4- [[3-[3-(trifluoromethyl)-1H- pyrazol-4-yl]imidazo[1,2- a]pyrazin-8-yl]amino]benzamide (example 10, step 3) with 2- chloroacetamide [CAS#79-07-2] |
| 358 | 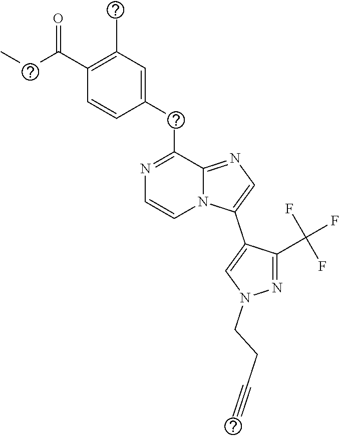 [SMILES: C#CCCn1cc(-c2cnc3c(Cc4ccc(C(=O)CC)c(C)c4)nccn23)c(C(F)(F)F)n1] | 2-chloro-4-[[3- [1-(2-cyanoethyl)- 3- (trifluoromethyl) pyrazol-4- yl]imidazo[1,2- a]pyrazin-8- yl]amino]-N- methylbenzamide | 488.9 | 489.3 | In analogy to the procedure described for example 2: alkylation of 2-chloro-N-methyl-4- [[3-[3-(trifluoromethyl)-1H- pyrazol-4-yl]imidazo[1,2- a]pyrazin-8-yl]amino]benzamide (example 10, step 3) with 3- bromopropanenitrile [CAS#2417- 90-5] |
| 359 | 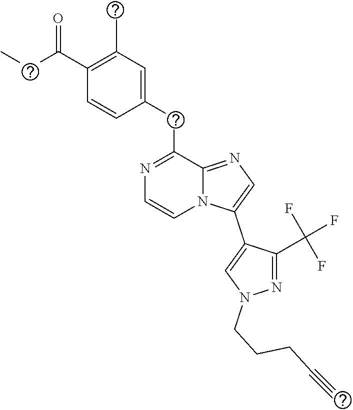 [SMILES: C#CCCCn1cc(-c2cnc3c(Cc4ccc(C(=O)CC)c(C)c4)nccn23)c(C(F)(F)F)n1] | 2-chloro-4-[[3- [1-(3- cyanopropyl)-3- (trifluoromethyl) pyrazol-4- yl]imidazo[1,2- a]pyrazin-8- yl]amino]-N- methylbenzamide | 502.9 | 503.3 | In analogy to the procedure described for example 2: alkylation of 2-chloro-N-methyl-4- [[3-[3-(trifluoromethyl)-1H- pyrazol-4-yl]imidazo[1,2- a]pyrazin-8-yl]amino]benzamide (example 10, step 3) with 4- bromobutanenitrile [CAS#5332- 06-9] |
| 360 | 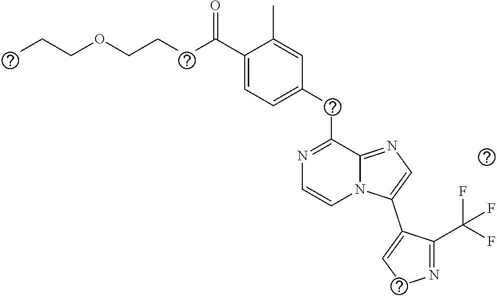 [SMILES: CCCOCCCC(=O)c1ccc(Cc2nccn3c(C4=CCN=C4C(F)(F)F)cnc23)cc1C] | N-[2-(2- aminoethoxy)ethyl]- 2-methyl-4-[[3- [3- (trifluoromethyl)- 1H-pyrazol-4- yl]imidazo[1,2- a]pyrazin-8- yl]amino]benzamide hydrochloride | 488.5 | 489.3 | in analogy to procedures described for example 1: through acidic Boc deprotection of tert-butyl N-[2-[2- [[2-methyl-4-[[3-[3- (trifluoromethyl)-1H-pyrazol-4- yl]imidazo[1,2-a]pyrazin-8- yl]amino]benzoyl]amino]ethoxy] ethyl]carbamate (example 16, step 3) |
| 361 | 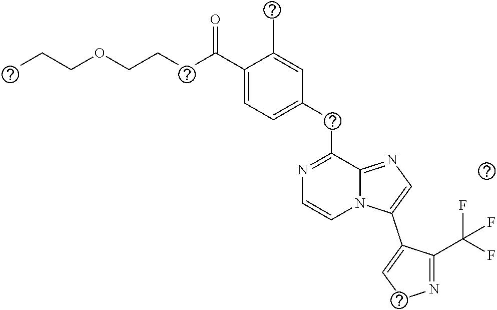 [SMILES: CCCOCCCC(=O)c1ccc(Cc2nccn3c(C4=CCN=C4C(F)(F)F)cnc23)cc1C] | N-[2-(2- aminoethoxy)ethyl]- 2-chloro-4-[[3- [3- (trifluoromethyl)- 1H-pyrazol-4- yl]imidazo[1,2- a]pyrazin-8- yl]amino]benzamide hydrochloride | 508.9 | 509.3 | in analogy to the procedure for example 1/2: amide formation from 2-chloro-4-[(3- iodoimidazo[1,2-a]pyrazin-8- yl)amino]benzoic acid (example 14, step 1) and tert-butyl (2-(2- aminoethoxy)ethyl)carbamate, followed by Suzuki reaction with (3-(trifluoromethyl)-1H-pyrazol-4- yl)boronic acid and subsequent acidic cleavage of the Boc group |
| 362 | 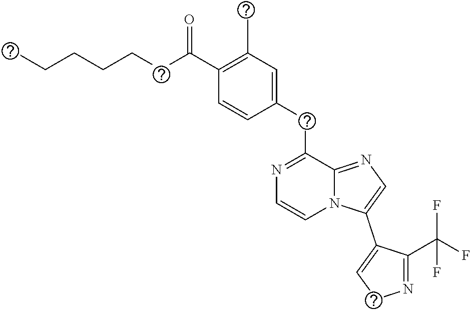 [SMILES: CCCCCCC(=O)c1ccc(Cc2nccn3c(C4=CCN=C4C(F)(F)F)cnc23)cc1C] | N-(4- aminobutyl)-2- chloro-4-[[3-[3- (trifluoromethyl)- 1H-pyrazol-4- yl]imidazo[1,2- a]pyrazin-8- yl]amino]benzamide | 492.9 | 493.4 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (4-aminobutyl)carbamate and subsequent acidic Boc deprotection |
| 363 | 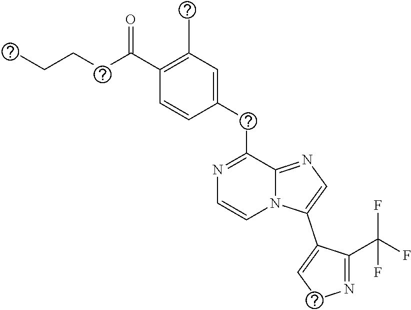 [SMILES: CCCCC(=O)c1ccc(Cc2nccn3c(C4=CCN=C4C(F)(F)F)cnc23)cc1C] | N-(2- aminoethyl)-2- chloro-4-[[3-[3- (trifluoromethyl)- 1H-pyrazol-4- yl]imidazo[1,2- a]pyrazin-8- yl]amino]benzamide | 464.8 | 465.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (4-aminoethyl)carbamate and subsequent acidic Boc deprotection |
| 364 | 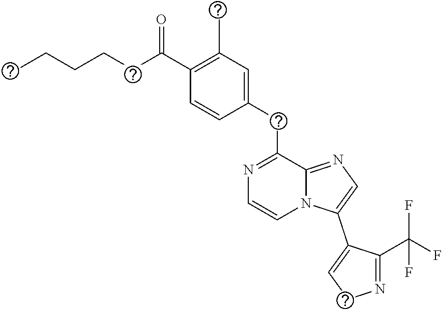 [SMILES: CCCCCC(=O)c1ccc(Cc2nccn3c(C4=CCN=C4C(F)(F)F)cnc23)cc1C] | N-(3- aminopropyl)-2- chloro-4-[[3-[3- (trifluoromethyl)- 1H-pyrazol-4- yl]imidazo[1,2- a]pyrazin-8- yl]amino]benzamide | 478.9 | 479.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl (4-aminopropyl)carbamate and subsequent acidic Boc deprotection |
| 365 | 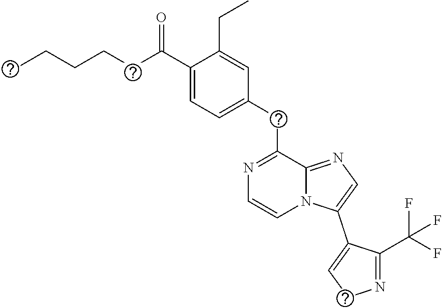 [SMILES: CCCCCC(=O)c1ccc(Cc2nccn3c(C4=CCN=C4C(F)(F)F)cnc23)cc1CC] | N-(3- aminopropyl)-2- ethyl-4-[[3-[3- (trifluoromethyl)- 1H-pyrazol-4- yl]imidazo[1,2- a]pyrazin-8- yl]amino]benzamide hydrochloride | 472.5 | 473.3 | in analogy to procedures for example 1/2: amide formation from 2-ethyl-4-[(3- iodoimidazo[1,2-a]pyrazin-8- yl)amino]benzoic acid, hydrochloride (example 1, step 2) and tert-butyl (4- aminopropyl)carbamate, followed by Suzuki reaction with (3- (trifluoromethyl)-1H-pyrazol-4- yl)boronic acid and subsequent acidic cleavage of the Boc group |
| 366 | 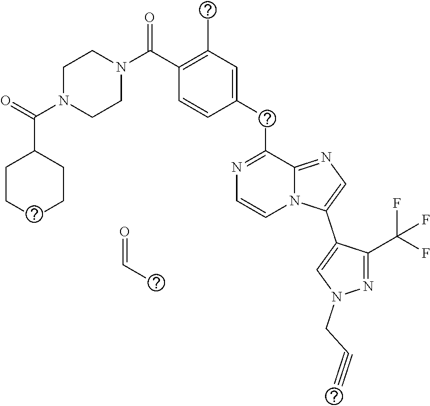 | 2-[4-[8-[3- chloro-4-[4- (piperidine-4- carbonyl)piperazine- 1- carbonyl]anilino] imidazo[1,2- a]pyrazin-3-yl]-3- (trifluoromethyl) pyrazol-1- yl]acetonitrile formate | 641.0 | 641.2 | in analogy to procedures for example 12: amide coupling of 2- [4-[8-[3-chloro-4-(piperazine-1- carbonyl)anilino]imidazo[1,2- a]pyrazin-3-yl]-3- (trifluoromethyl)pyrazol-1- yl]acetonitrile (example 12, step 2) and N-Boc-isonipecotic acid followed by acidic Boc deprotection |
| 367 | 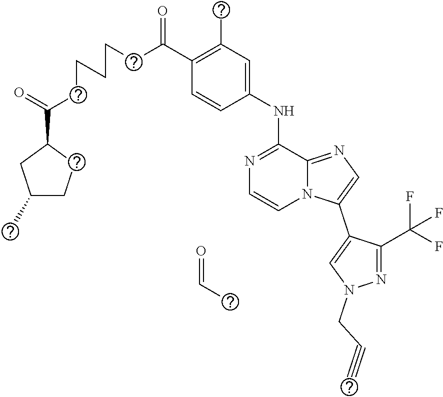 | (2S,4R)-N-[3-[[4- [[3-[1- (cyanomethyl)-3- (trifluoromethyl) pyrazol-4- yl]imidazo[1,2- a]pyrazin-8- yl]amino]-2- methylbenzoyl] amino]propyl]-4- hydroxypyrrolidine- 2-carboxamide formate | 610.6 | 611.2 | in analogy to the procedures for example 1: amide coupling of 4- [(3-iodoimidazo[1,2-a]pyrazin-8- yl)amino]-2-methyl-benzoic acid (example 16, step 1) with N-Boc- 1,3-diaminopropane, subsequent Suzuki reaction with 2-[4-(4,4,5,5- tetramethyl-1,3-dioxolan-2-yl)-3- (trifluoromethyl)pyrazol-1- yl]acetonitrile, acidic Boc deprotection, coupling with BOC- HYP-OH and subsequent acidic Boc deprotection |
| 370 | 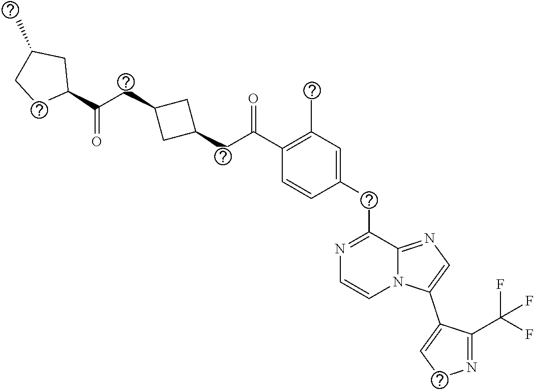 [SMILES: Cc1cc(Cc2nccn3c(C4=CCN=C4C(F)(F)F)cnc23)ccc1C(=O)C[C@H]1C[C@@H](CC(=O)[C@H]2CC[C@H](C)C2)C1] | (2S,4R)-N-[3-[[2- chloro-4-[[3-[3- (trifluoromethyl)- 1H-pyrazol-4- yl]imidazo[1,2- a]pyrazin-8- yl]amino]benzoyl] amino]cyclobutyl]- 4- hydroxypyrrolidine- 2-carboxamide | 604.0 | 604.3 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl ((1S,3S)-3- aminocyclobutyl)carbamate, acidic Boc deprotection, coupling with (2S,4R)-1-(tert-butoxycarbonyl)-4- hydroxypyrrolidine-2-carboxylic acid and subsequent acidic Boc deprotection |
| 371 | 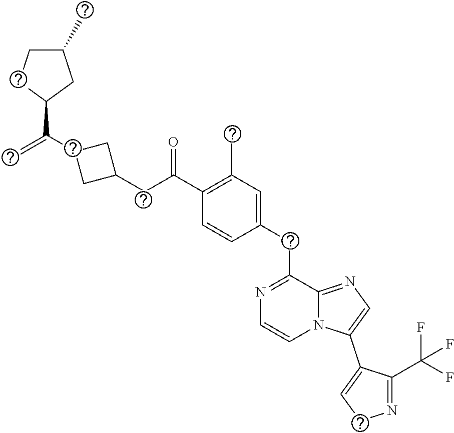 [SMILES: C=C(C1CC(CC(=O)c2ccc(Cc3nccn4c(C5=CCN=C5C(F)(F)F)cnc34)cc2C)C1)[C@H]1CC[C@H](C)C1] | 2-chloro-N-[1- [(2S,4R)-4- hydroxypyrrolidine- 2- carbonyl]azetidin- 3-yl]-4-[[3-[3- (trifluoromethyl)- 1H-pyrazol-4- yl]imidazo[1,2- a]pyrazin-8- yl]amino]benzamide | 590.0 | 590.3 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl 3-aminoazetidine-1-carboxylate, acidic Boc deprotection, coupling with (2S,4R)-1-(tert- butoxycarbonyl)-4- hydroxypyrrolidine-2-carboxylic acid and subsequent acidic Boc deprotection |
| 372 | 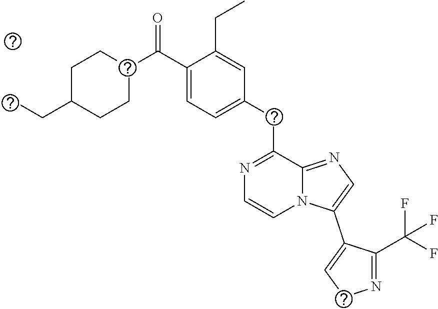 [SMILES: CCc1cc(Cc2nccn3c(C4=CCN=C4C(F)(F)F)cnc23)ccc1C(=O)C1CCC(CC)CC1] | [4- (aminomethyl) piperidin-1-yl]-[2- ethyl-4-[[3-[3- (trifluoromethyl)- 1H-pyrazol-4- yl]imidazo[1,2- a]pyrazin-8- yl]amino]phenyl] methanone hydrochloride | 512.5 | 513.5 | in analogy to the procedures for example 1: amide coupling of 2- ethyl-4-[(3-iodoimidazo[1,2- a]pyrazin-8-yl)amino]benzoic acid, hydrochloride with tert-butyl (piperidin-4-ylmethyl)carbamate, subsequent Suzuki reaction with (3-(trifluoromethyl)-1H-pyrazol-4- yl)boronic acid and acidic Boc deprotection |
| 373 | 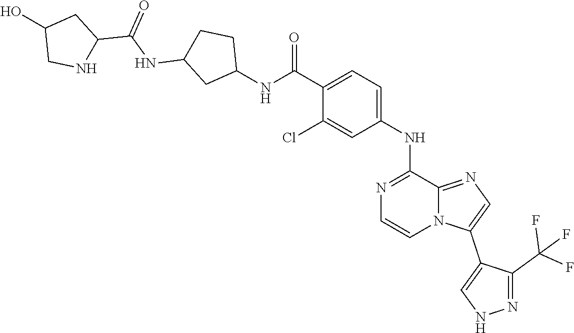 [SMILES: O=C(NC1CCC(NC(=O)C2CC(O)CN2)C1)c1ccc(Nc2nccn3c(-c4c[nH]nc4C(F)(F)F)cnc23)cc1Cl] | rac-(2R,4S)-N- [3-[[2-chloro-4- [[3-[3- (trifluoromethyl)- 1H-pyrazol-4- yl]imidazo[1,2- a]pyrazin-8- yl]amino]benzoyl] amino]cyclopentyl]- 4- hydroxypyrrolidine- 2-carboxamide | 618.0 | 618.3 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl ((1R,3R)-3- aminocyclopentyl)carbamate, acidic Boc deprotection, coupling with (2S,4R)-1-(tert- butoxycarbonyl)-4- hydroxypyrrolidine-2-carboxylic acid and subsequent acidic Boc deprotection |
| 374 | 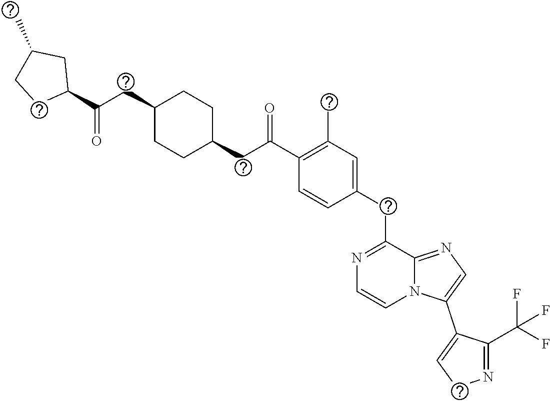 [SMILES: Cc1cc(Cc2nccn3c(C4=CCN=C4C(F)(F)F)cnc23)ccc1C(=O)C[C@H]1CC[C@@H](CC(=O)[C@H]2CC[C@H](C)C2)CC1] | (2S,4R)-N-[4-[[2- chloro-4-[[3-[3- (trifluoromethyl)- 1H-pyrazol-4- yl]imidazo[1,2- a]pyrazin-8- yl]amino]benzoyl] amino]cyclohexyl]- 4- hydroxypyrrolidine- 2-carboxamide | 632.0 | 632.4 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with ((1S,3S)- 4-aminocyclohexyl)carbamate acid, acidic Boc deprotection, coupling with (2S,4R)-1-(tert- butoxycarbonyl)-4- hydroxypyrrolidine-2-carboxylic acid and subsequent acidic Boc deprotection |
| 375 | 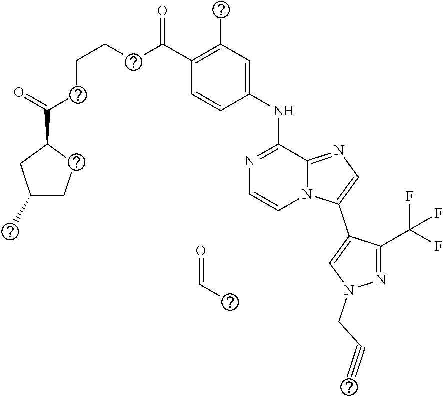 | (2S,4R)-N-[2-[[4- [[3-[1- (cyanomethyl)-3- (trifluoromethyl) pyrazol-4- yl]imidazo[1,2- a]pyrazin-8- yl]amino]-2- methylbenzoyl] amino]ethyl]-4- hydroxypyrrolidine- 2-carboxamide formate | 596.6 | 597.1 | in analogy to the procedures for example 1: amide coupling of 4- [(3-iodoimidazo[1,2-a]pyrazin-8- yl)amino]-2-methyl-benzoic acid (example 16, step 1) with N-Boc- 1,2-diaminoethane, subsequent Suzuki reaction with 2-[4-(4,4,5,5- tetramethyl-1,3-dioxolan-2-yl)-3- (trifluoromethyl)pyrazol-1- yl]acetonitrile, acidic Boc deprotection, coupling with (2S,4R)-1-(tert-butoxycarbonyl)-4- hydroxypyrrolidine-2-carboxylic acid and subsequent acidic Boc deprotection |
| 376 | 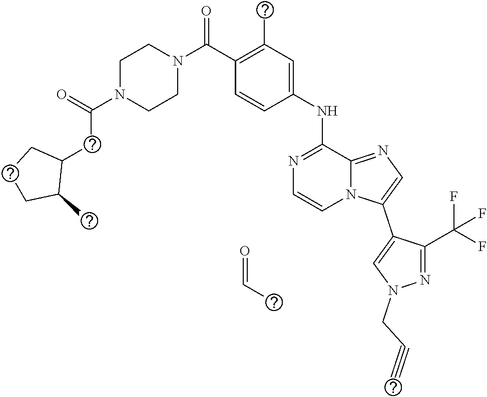 | 4-[4-[[3-[1- (cyanomethyl)-3- (trifluoromethyl) pyrazol-4- yl]imidazo[1,2- a]pyrazin-8- yl]amino]-2- methylbenzoyl]- N-[(3S,4S)-4- hydroxypyrrolidin- 3-yl]piperazine- 1-carboxamide formate | 637.6 | 638.3 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with tert- butyl trans-3-amino-4-hydroxy-1- pyrrolidinecarboxylate and subsequent acidic Boc deprotection |
| 378 | 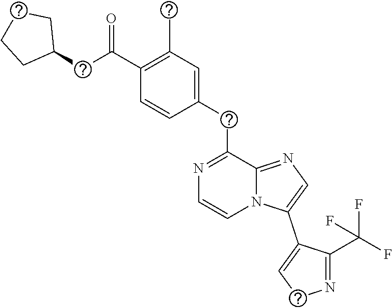 | 2-chloro-N-[(3S)- pyrrolidin-3-yl]-4- [[3-[3- (trifluoromethyl)- 1H-pyrazol-4- yl]imidazo[1,2- a]pyrazin-8- yl]amino]benzamide | 490.9 | 491.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl (S)-3-aminopyrrolidine-1- carboxylate and subsequent acidic Boc deprotection |
| 379 | 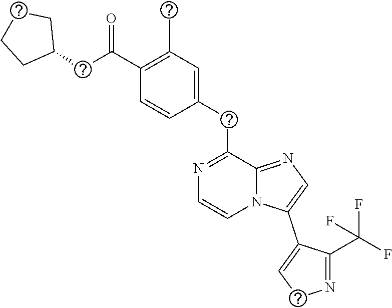 | 2-chloro-N- [(3R)-pyrrolidin- 3-yl]-4-[[3-[3- (trifluoromethyl)- 1H-pyrazol-4- yl]imidazo[1,2- a]pyrazin-8- yl]amino]benzamide | 490.9 | 491.3 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl (R)-3-aminopyrrolidine-1- carboxylate and subsequent acidic Boc deprotection |
| 380 | 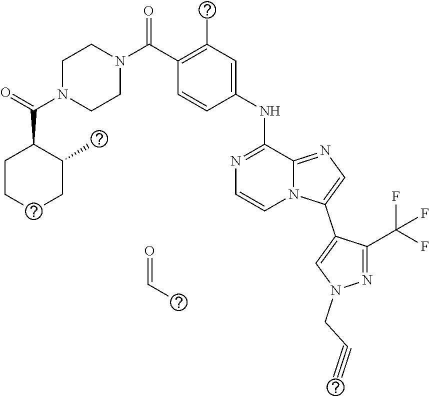 | formic acid; 2-[4- [8-[4-[4-[(3S,4R)- 3- hydroxypiperidine- 4- carbonyl]piperazine- 1-carbonyl]-3- methylanilino] imidazo[1,2- a]pyrazin-3-yl]-3- (trifluoromethyl) pyrazol-1- yl]acetonitrile | 636.6 | 637.1 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with (3S,4R)- 1-tert-butoxycarbonyl-3-hydroxy- piperidine-4-carboxylic acid and subsequent acidic Boc deprotection |
| 381 | 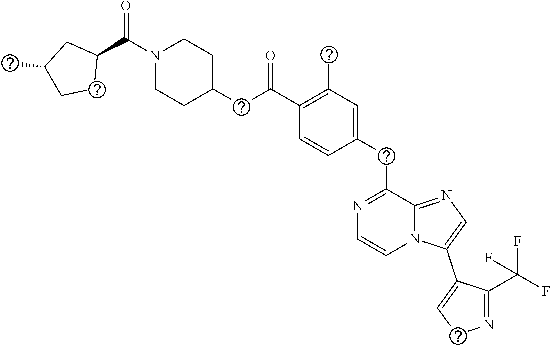 [SMILES: Cc1cc(Cc2nccn3c(C4=CCN=C4C(F)(F)F)cnc23)ccc1C(=O)CC1CCN(C(=O)[C@H]2CC[C@H](C)C2)CC1] | 2-chloro-N-[1- [(2S,4R)-4- hydroxypyrrolidine- 2- carbonyl]piperidin- 4-yl]-4-[[3-[3- (trifluoromethyl)- 1H-pyrazol-4- yl]imidazo[1,2- a]pyrazin-8- yl]amino]benzamide | 618.0 | 618.3 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (2S,4R)- 1-(tert-butoxycarbonyl)-4- hydroxypyrrolidine-2-carboxylic acid and acidic Boc deprotection |
| 382 | 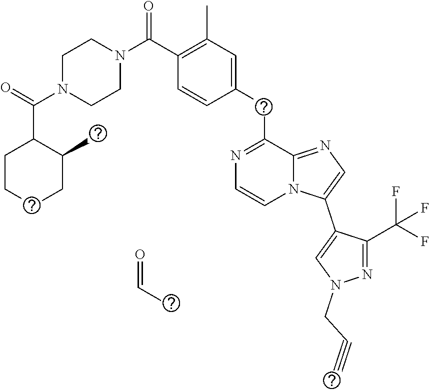 | formic acid; 2-[4- [8-[4-[4-[(3R,4S)- 3- hydroxypiperidine- 4- carbonyl]piperazine- 1-carbonyl]-3- methylanilino] imidazo[1,2- a]pyrazin-3-yl]-3- (trifluoromethyl) pyrazol-1- yl]acetonitrile | 636.6 | 637.3 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with (3R,4S)- 1-tert-butoxycarbonyl-3-hydroxy- piperidine-4-carboxylic acid and subsequent acidic Boc deprotection |
| 384 | 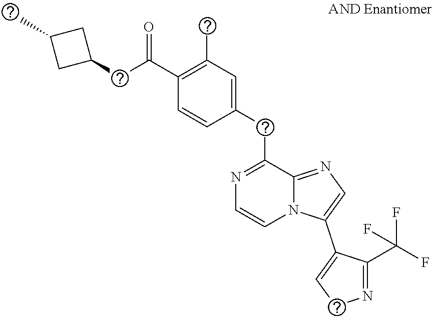 [SMILES: Cc1cc(Cc2nccn3c(C4=CCN=C4C(F)(F)F)cnc23)ccc1C(=O)C[C@H]1C[C@H](C)C1] | N-(3- aminocyclobutyl)- 2-chloro-4-[[3-[3- (trifluoromethyl)- 1H-pyrazol-4- yl]imidazo[1,2- a]pyrazin-8- yl]amino]benzamide | 490.9 | 491.3 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl ((1S,3S)-3- aminocyclobutyl)carbamate and subsequent acidic Boc deprotection |
| 385 | 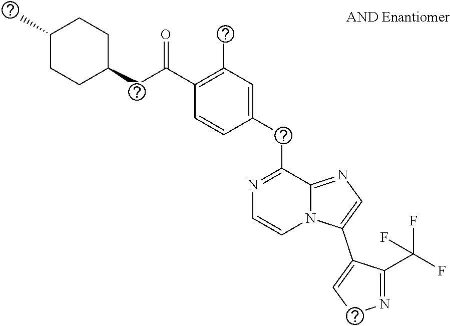 [SMILES: Cc1cc(Cc2nccn3c(C4=CCN=C4C(F)(F)F)cnc23)ccc1C(=O)C[C@H]1CC[C@H](C)CC1] | N-(4- aminocyclohexyl)- 2-chloro-4-[[3- [3- (trifluoromethyl)- 1H-pyrazol-4- yl]imidazo[1,2- a]pyrazin-8- yl]amino]benzamide | 518.9 | 519.4 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl ((1R,4R)-4- aminocyclohexyl)carbamate and subsequent acidic Boc deprotection |
| 387 | 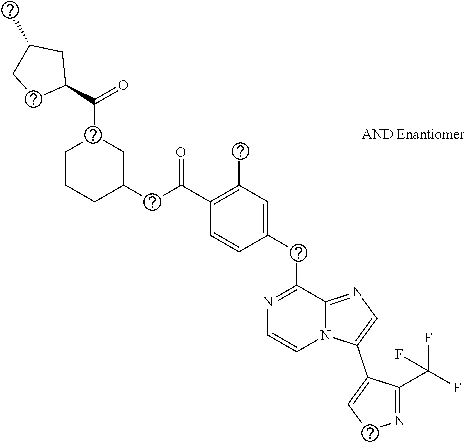 [SMILES: Cc1cc(Cc2nccn3c(C4=CCN=C4C(F)(F)F)cnc23)ccc1C(=O)CC1CCCC(C(=O)[C@H]2CC[C@H](C)C2)C1] | 2-chloro-N-[1- [rac-(2R,4S)-4- hydroxypyrrolidine- 2- carbonyl]piperidine- 3-yl]-4-[[3-[3- (trifluoromethyl)- 1H-pyrazol-4- yl]imidazo[1,2- a]pyrazin-8- yl]amino]benzamide | 618.0 | 618.5 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl 3-aminopiperidine-1-carboxylate, acidic Boc deprotection, coupling with (2S,4R)-1-(tert- butoxycarbonyl)-4- hydroxypyrrolidine-2-carboxylic acid and subsequent acidic Boc deprotection |
| 388 | 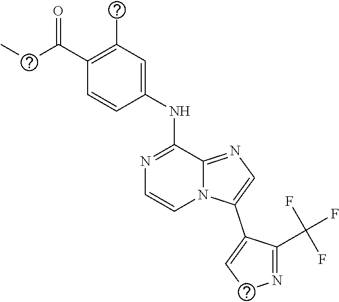 [SMILES: CCC(=O)c1ccc(Nc2nccn3c(C4=CCN=C4C(F)(F)F)cnc23)cc1C] | 2-chloro-N- methyl-4-[[3-[3- (trifluoromethyl)- 1H-pyrazol-4- yl]imidazo[1,2- a]pyrazin-8- yl]amino]benzamide | 435.8 | 436.3 | example 10, step 2 |
| 389 | 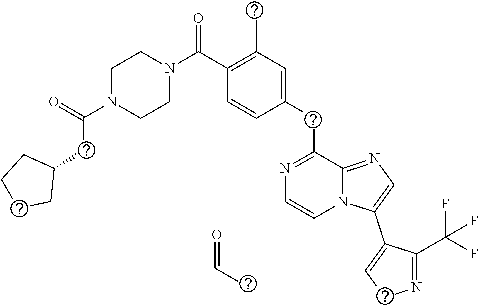 | 4-[2-chloro-4- [[3-[3- (trifluoromethyl)- 1H-pyrazol-4- yl]imidazo[1,2- a]pyrazin-8- yl]amino]benzoyl]- N-[(3S)- pyrrolidin-3- yl]piperazine-1- carboxamide formate | 603.0 | 603.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (S)-3-amino-1-N-Boc-pyrrolidine followed by acidic Boc deprotection |
| 390 | 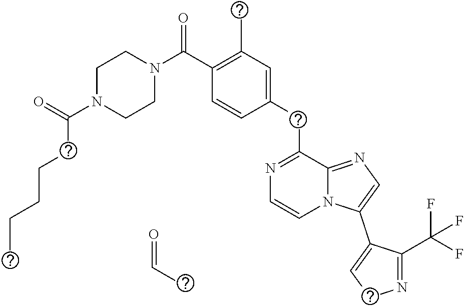 | N-(3- aminopropyl)-4- [2-chloro-4-[[3- [3- (trifluoromethyl)- 1H-pyrazol-4- yl]imidazo[1,2- a]pyrazin-8- yl]amino]benzoyl] piperazine-1- carboxamide formate | 591.0 | 591.1 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and N-Boc-1,3-diaminopropane followed by acidic Boc deprotection |
| 391 | 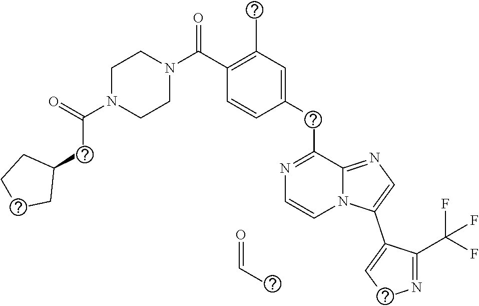 | 4-[2-chloro-4- [[3-[3- (trifluoromethyl)- 1H-pyrazol-4- yl]imidazo[1,2- a]pyrazin-8- yl]amino]benzoyl]- N-[(3R)- pyrrolidin-3- yl]piperazine-1- carboxamide formate | 603.0 | 603.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and (R)-3-amino-1-N-Boc-pyrrolidine followed by acidic Boc deprotection |
| 392 | 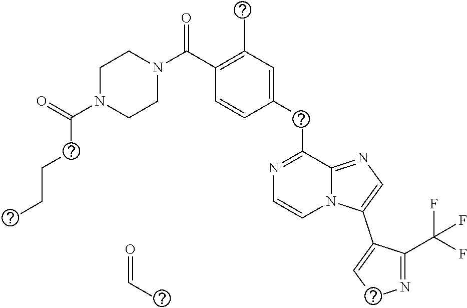 | N-(2- aminoethyl)-4-[2- chloro-4-[[3-[3- (trifluoromethyl)- 1H-pyrazol-4- yl]imidazo[1,2- a]pyrazin-8- yl]amino]benzoyl] piperazine-1- carboxamide formate | 577.0 | 577.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and N-Boc-1,3-diaminoethane followed by acidic Boc deprotection |
| 393 | 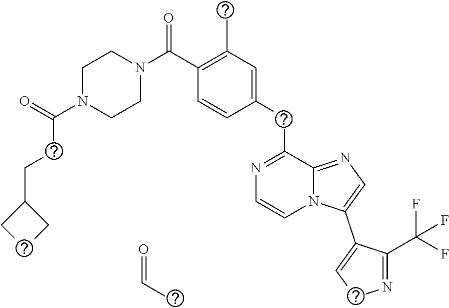 | N-(azetidin-3- ylmethyl)-4-[2- chloro-4-[[3-[3- (trifluoromethyl)- 1H-pyrazol-4- yl]imidazo[1,2- a]pyrazin-8- yl]amino]benzoyl] piperazine-1- carboxamide formate | 603.0 | 603.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and 1-Boc-3-(aminomethyl)azetidine followed by acidic Boc deprotection |
| 394 | 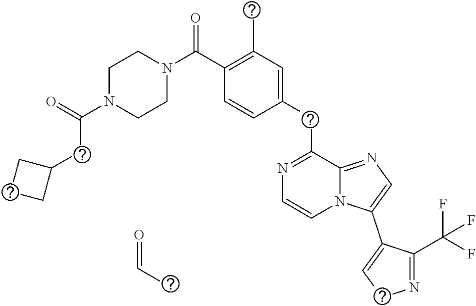 | N-(azetidin-3-yl)- 4-[2-chloro-4-[[3- [3- (trifluoromethyl)- 1H-pyrazol-4- yl]imidazo[1,2- a]pyrazin-8- yl]amino]benzoyl] piperazine-1- carboxamide formate | 589.0 | 589.1 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and 1-Boc-3-(amino)azetidine followed by acidic Boc deprotection |
| 399 | 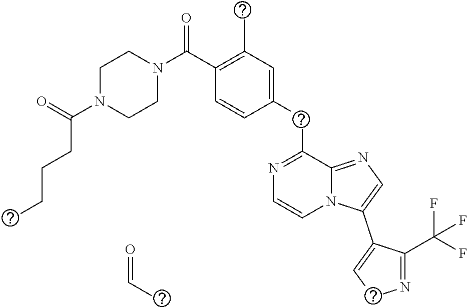 | 4-amino-1-[4-[2- chloro-4-[[3-[3- (trifluoromethyl)- 1H-pyrazol-4- yl]imidazo[1,2- a]pyrazin-8- yl]amino]benzoyl] piperazin-1- yl]butan-1-one formate | 576.0 | 576.3 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with Boc- gamma-abu-OH and acidic Boc deprotection |
| 400 | 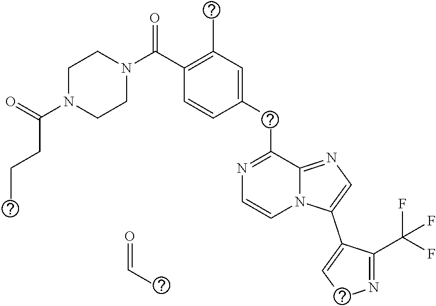 | 3-amino-1-[4-[2- chloro-4-[[3-[3- (trifluoromethyl)- 1H-pyrazol-4- yl]imidazo[1,2- a]pyrazin-8- yl]amino]benzoyl] piperazin-1- yl]propan-1-one formate | 561.9 | 562.2 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with Boc- BETA-ALA-OH and acidic Boc deprotection |
| 401 | 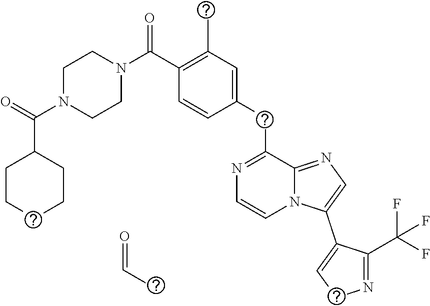 | [2-chloro-4-[[3- [3- (trifluoromethyl)- 1H-pyrazol-4- yl]imidazo[1,2- a]pyrazin-8- yl]amino]phenyl]- [4-(piperidine-4- carbonyl)piperazin- 1-yl]methanone formate | 602.0 | 602.1 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with N-Boc- isonipecotic acid and acidic Boc deprotection |
| 402 | 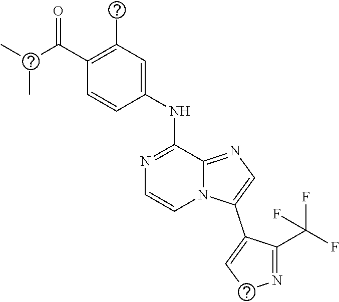 [SMILES: Cc1cc(Nc2nccn3c(C4=CCN=C4C(F)(F)F)cnc23)ccc1C(=O)C(C)C] | 2-chloro-N,N- dimethyl-4-[[3-[3- (trifluoromethyl)- 1H-pyrazol-4- yl]imidazo[1,2- a]pyrazin-8- yl]amino]benzamide | 449.8 | 450.2 | in analogy to the procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with dimethyl amine |
| 403 | 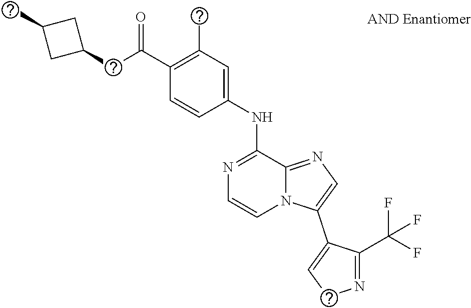 [SMILES: Cc1cc(Nc2nccn3c(C4=CCN=C4C(F)(F)F)cnc23)ccc1C(=O)C[C@H]1C[C@@H](C)C1] | N-(3- aminocyclobutyl)- 2-chloro-4-[[3-[3- (trifluoromethyl)- 1H-pyrazol-4- yl]imidazo[1,2- a]pyrazin-8- yl]amino]benzamide | 490.9 | 491.2 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl ((1S,3S)-3- aminocyclobutyl)carbamate and acidic Boc deprotection |
| 404 | 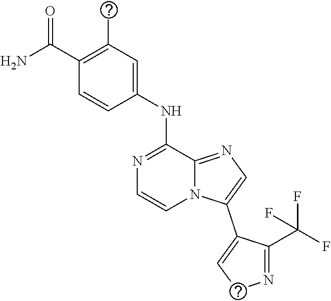 [SMILES: Cc1cc(Nc2nccn3c(C4=CCN=C4C(F)(F)F)cnc23)ccc1C(N)=O] | 2-chloro-4-[[3- [3- (trifluoromethyl)- 1H-pyrazol-4- yl]imidazo[1,2- a]pyrazin-8- yl]amino]benzamide | 421.8 | 422.2 | in analogy to procedures for example 3: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with 25% ammonia in water |
| 405 | 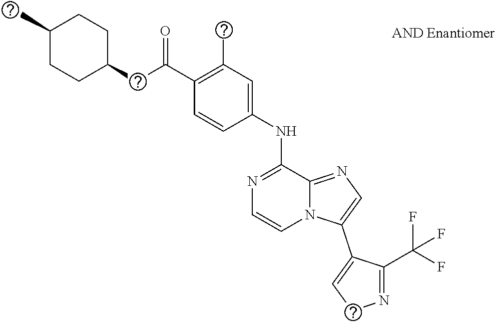 [SMILES: Cc1cc(Nc2nccn3c(C4=CCN=C4C(F)(F)F)cnc23)ccc1C(=O)C[C@H]1CC[C@@H](C)CC1] | N-(4- aminocyclohexyl)- 2-chloro-4-[[3- [3- (trifluoromethyl)- 1H-pyrazol-4- yl]imidazo[1,2- a]pyrazin-8- yl]amino]benzamide | 518.9 | 519.3 | in analogy to the procedures for example 1: amide coupling of 2- chloro-4-[[3-[3-(trifluoromethyl)- 1H-pyrazol-4-yl]imidazo[12- a]pyrazin-8-yl]amino]benzoic acid (example 6, step 3) with ((1S,4S)- 4-aminocyclohexyl)carbamate acid and acidic Boc deprotection |
| 406 | 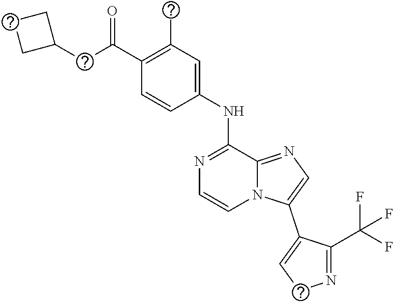 [SMILES: Cc1cc(Nc2nccn3c(C4=CCN=C4C(F)(F)F)cnc23)ccc1C(=O)CC1CCC1] | N-(azetidin-3-yl)- 2-chloro-4-[[3-[3- (trifluoromethyl)- 1H-pyrazol-4- yl]imidazo[1,2- a]pyrazin-8- yl]amino]benzamide | 476.8 | 477.4 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl 3-aminoazetidine-1-carboxylate and acidic Boc deprotection |
| 407 | 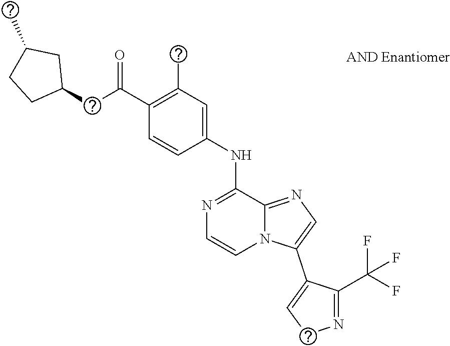 [SMILES: Cc1cc(Nc2nccn3c(C4=CCN=C4C(F)(F)F)cnc23)ccc1C(=O)C[C@H]1CC[C@H](C)C1] | 2-chloro-N-[rac- (1R,3R)-3- aminocyclopentyl]- 4-[[3-[3- (trifluoromethyl)- 1H-pyrazol-4- yl]imidazo[1,2- a]pyrazin-8- yl]amino]benzamide | 504.9 | 505.3 | in analogy to the procedures for example 1: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with rac-tert- butyl ((1R,3R)-3- aminocyclopentyl)carbamate and acidic Boc deprotection |
| 408 | 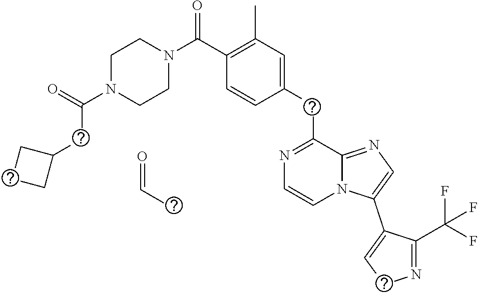 | N-(azetidin-3-yl)- 4-[2-methyl-4-[[3- [3- (trifluoromethyl)- 1H-pyrazol-4- yl]imidazo[1,2- a]pyrazin-8- yl]amino]benzoyl] piperazine-1- carboxamide formate | 568.6 | 569.1 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-Boc-3- (amino)azetidine and acidic Boc protection |
| 410 | 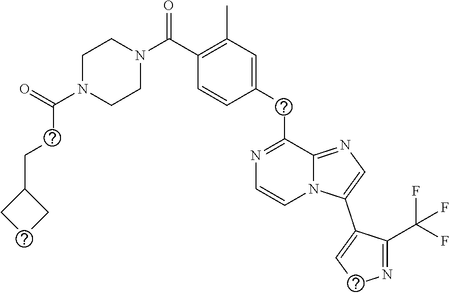 [SMILES: Cc1cc(Cc2nccn3c(C4=CCN=C4C(F)(F)F)cnc23)ccc1C(=O)N1CCN(C(=O)CCC2CCC2)CC1] | N-(azetidin-3- ylmethyl)-4-[2- methyl-4-[[3-[3- (trifluoromethyl)- 1H-pyrazol-4- yl]imidazo[1,2- a]pyrazin-8- yl]amino]benzoyl] piperazine-1- carboxamide | 582.6 | 583.2 | In analogy to procedures described for example 17, coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-Boc-3- (aminomethyl)azetidine and acidic Boc deprotection |
| 413 | 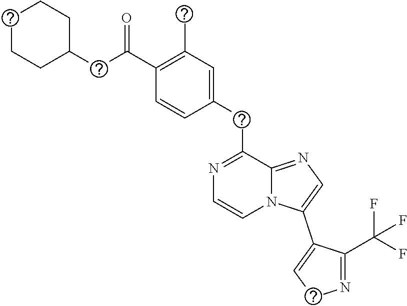 [SMILES: Cc1cc(Cc2nccn3c(C4=CCN=C4C(F)(F)F)cnc23)ccc1C(=O)CC1CCCCC1] | 2-chloro-N- piperidin-4-yl-4- [[3-[3- (trifluoromethyl)- 1H-pyrazol-4- yl]imidazo[1,2- a]pyrazin-8- yl]amino]benzamide | 504.9 | 505.3 | in analogy to procedures for example 6: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl 4-aminopiperidine-1-carboxylate and acidic Boc deprotection |
| 414 | 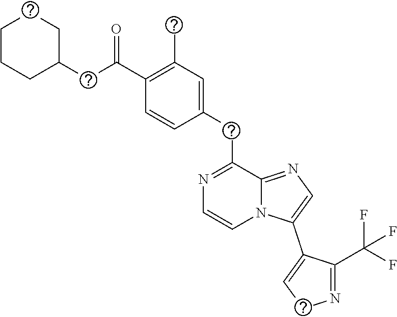 [SMILES: Cc1cc(Cc2nccn3c(C4=CCN=C4C(F)(F)F)cnc23)ccc1C(=O)CC1CCCCC1] | 2-chloro-N- piperidin-3-yl-4- [[3-[3- (trifluoromethyl)- 1H-pyrazol-4- yl]imidazo[1,2- a]pyrazin-8- yl]amino]benzamide | 504.9 | 505.3 | in analogy to procedures for example 6: amide coupling of 2- chloro-4-[[3-[3-(trifluoromethyl)- 1H-pyrazol-4-yl]imidazo[1,2- a]pyrazin-8-yl]amino]benzoic acid (example 6, step 3) with tert-butyl 3-aminopiperidine-1-carboxylate and acidic Boc deprotection |
| 415 | 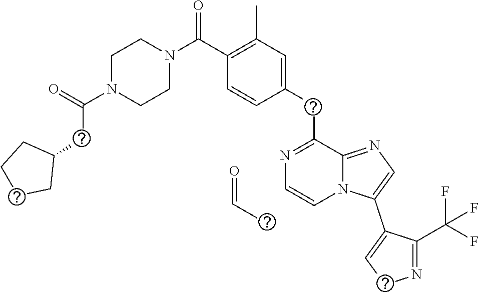 | formic acid; 4-[2- methyl-4-[[3-[3- (trifluoromethyl)- 1H-pyrazol-4- yl]imidazo[1,2- a]pyrazin-8- yl]amino]benzoyl]- N-[(3S)- pyrrolidin-3- yl]piperidine-1- carboxamide | 582.6 | 583.1 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-Boc-3- (amino)azetidine and acidic Boc deprotection |
| 416 | 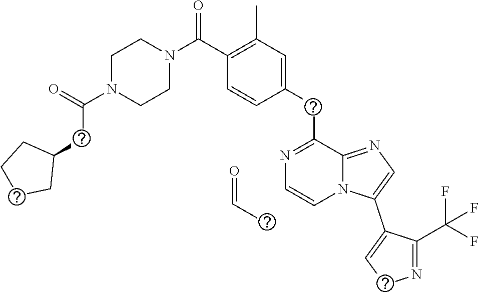 | formic acid; 4-[2- methyl-4-[[3-[3- (trifluoromethyl)- 1H-pyrazol-4- yl]imidazo[1,2- a]pyrazin-8- yl]amino]benzoyl]- N-[(3R)- pyrrolidin-3- yl]piperazine-1- carboxamide | 582.6 | 583.1 | in analogy to the procedures for example 12: urea formation [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (R)-(+)- 1-Boc-3-aminopyrrolidine followed by acidic Boc deprotection |
| 417 | 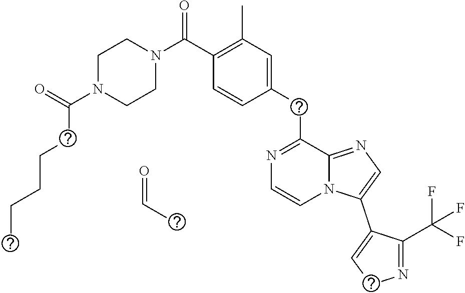 | N-(3- aminopropyl)-4- [2-methyl-4-[[3- [3- (trifluoromethyl)- 1H-pyrazol-4- yl]imidazo[1,2- a]pyrazin-8- yl]amino]benzoyl] piperazine-1- carboxamide formate | 570.6 | 571.3 | in analogy to the procedures for example 12: urea formation of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with N-Boc- 1,3-diaminopropane followed by acidic Boc deprotection |
| 418 | 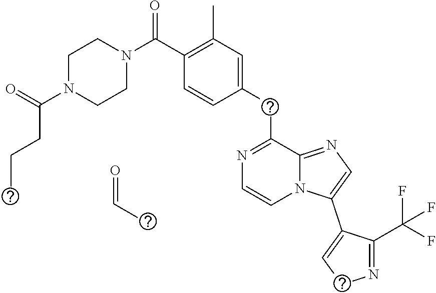 | 3-amino-1-[4-[2- methyl-4-[[3-[3- (trifluoromethyl)- 1H-pyrazol-4- yl]imidazo[1,2- a]pyrazin-8- yl]amino]benzoyl] piperazin-1- yl]propan-1-one formate | 541.5 | 542.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with Boc- BETA-ALA-OH and acidic Boc deprotection |
| 419 | 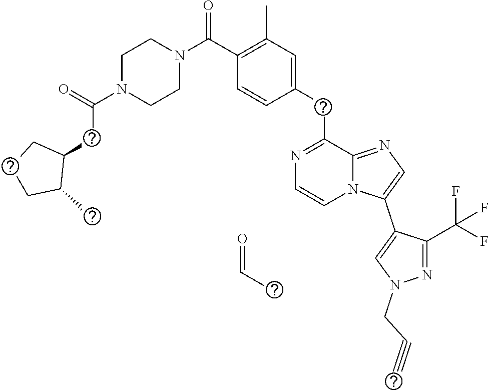 | 4-[4-[[3-[1- (cyanomethyl)-3- (trifluoromethyl) pyrazol-4- yl]imidazo[1,2- a]pyrazin-8- yl]amino]-2- methylbenzoyl]- N-[(3R,4R)-4- hydroxypyrrolidin- 3-yl]piperazine- 1-carboxamide formate | 637.6 | 638.3 | In analogy to the procedure described for example 12: urea formation of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with tert- butyl (3R,4R)-3-amino-4-hydroxy- pyrrolidine-1-carboxylate and subsequent acidic Boc deprotection |
| 420 | 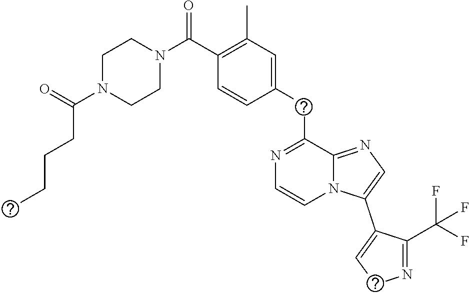 [SMILES: CCCCC(=O)N1CCN(C(=O)c2ccc(Cc3nccn4c(C5=CCN=C5C(F)(F)F)cnc34)cc2C)CC1] | 4-amino-1-[4-[2- methyl-4-[[3-[3- (trifluoromethyl)- 1H-pyrazol-4- yl]imidazo[1,2- a]pyrazin-8- yl]amino]benzoyl] piperazin-1- yl]butan-1-one | 555.6 | 556.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with Boc- gamma-abu-OH and acidic Boc deprotection |
| 421 | 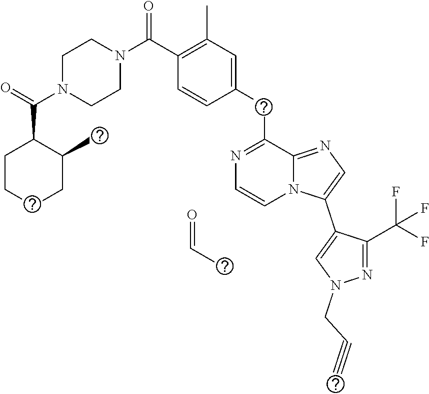 | formic acid; 2-[4- [8-[4-[4-[(3R,4R)- 3- hydroxypiperidine- 4- carbonyl]piperazine- 1-carbonyl]-3- methylanilino] imidazo[1,2- a]pyrazin-3-yl]-3- (trifluoromethyl) pyrazol-1- yl]acetonitrile | 636.6 | 637.1 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with (3R,4R)- 1-tert-butoxycarbonyl-3-hydroxy- piperidine-4-carboxylic acid and subsequent acidic Boc deprotection |
| 422 | 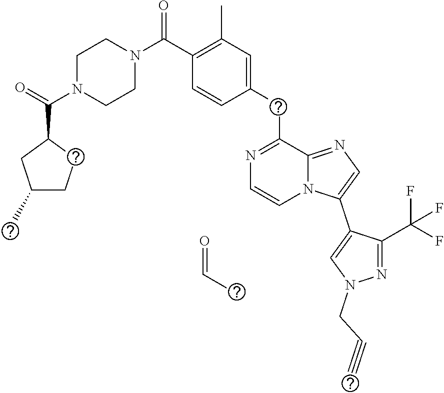 | formic acid; 2-[4- [8-[4-[4-[(2S,4R)- 4- hydroxypyrrolidine- 2- carbonyl]piperazine- 1-carbonyl]-3- methylanilino] imidazo[1,2- a]pyrazin-3-yl]-3- (trifluoromethyl) pyrazol-1- yl]acetonitrile | 622.6 | 623.3 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with Boc- HYP-OH and subsequent acidic Boc deprotection |
| 423 | 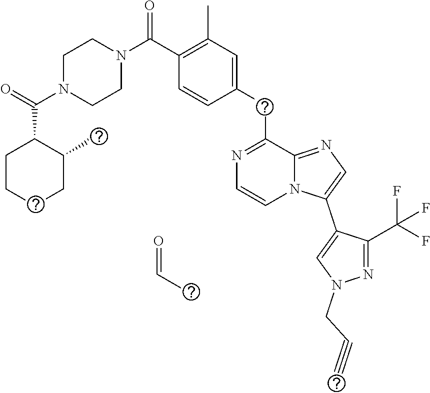 | formic acid; 2-[4- [8-[4-[4-[(3S,4S)- 3- hydroxypyrrolidine- 4- carbonyl]piperazine- 1-carbonyl]-3- methylanilino] imidazo[1,2- a]pyrazin-3-yl]-3- (trifluoromethyl) pyrazol-1- yl]acetonitrile | 636.6 | 637.1 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with (3S,4S)- 1-tert-butoxycarbonyl-3-hydroxy- piperidine-4-carboxylic acid and subsequent acidic Boc deprotection |
| 424 | 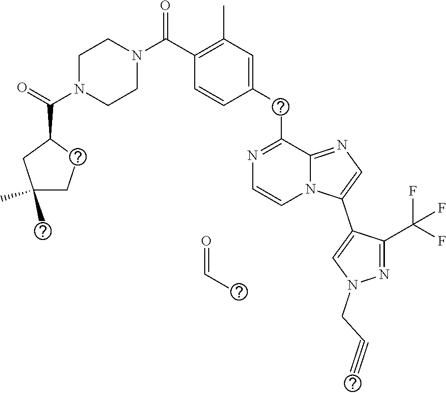 | formic acid; 2-[4- [8-[4-[4-[(2S,4S)- 4-hydroxy-4- methylpyrrolidine- 2- carbonyl]piperazine- 1-carbonyl]-3- methylanilino] imidazo[1,2- a]pyrazin-3-yl]-3- (trifluoromethyl) pyrazol-1- yl]acetonitrile | 636.6 | 637.3 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with (2S,4S)- 1-tert-butoxycarbonyl-4-hydroxy- 4-methyl-pyrrolidine-2-carboxylic acid and subsequent acidic Boc deprotection |
| 425 | 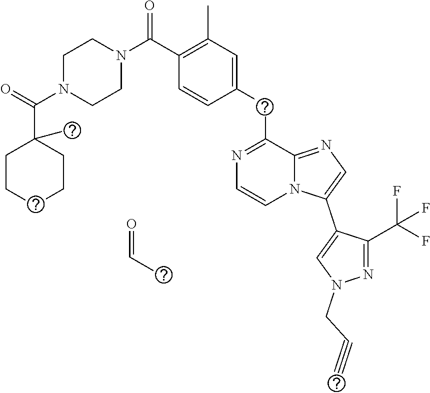 | formic acid; 2-[4- [8-[4-[4-(4- hydroxypiperidine- 4- carbonyl]piperazine- 1-carbonyl]-3- methylanilino] imidazo[1,2- a]pyrazin-3-yl]-3- (trifluoromethyl) pyrazol-1- yl]acetonitrile | 636.6 | 637.1 | In analogy to the procedure described for example 1: amide coupling of 4-[[3-[1- (cyanomethyl)-3- (trifluoromethyl)pyrazol-4- yl]imidazo[1,2-a]pyrazin-8- yl]amino]-2-methyl-benzoic acid (example 19, step 1) with 1-tert- butoxycarbonyl-4-hydroxy- piperidine-4-carboxylic acid and subsequent acidic Boc deprotection |
| 426 | 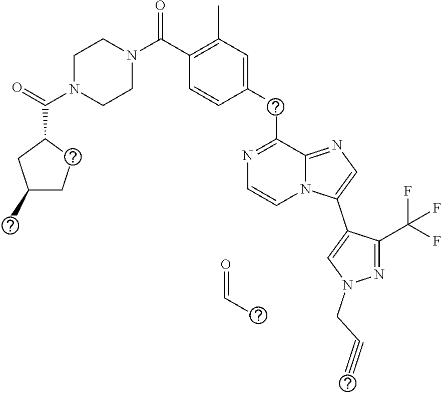 | formic acid; 2-[4- [8-[4-[4-[(2R,4S)- 4- hydroxypyrrolidine- 2- carbonyl]piperazine- 1-carbonyl]-3- methylanilino] imidazo[1,2- a]pyrazin-3-yl]-3- (trifluoromethyl) pyrazol-1- yl]acetonitrile | 622.6 | 623.1 | In analogy to the procedure described for example 1: amide coupling of [2-methyl-4-[[3-[3- (trifluoromethyl)-1H-pyrazol-4- yl]imidazo[1,2-a]pyrazin-8- yl]amino]phenyl]-piperazin-1-yl- methanone (example 17, step 3) with (2R,4S)-1-tert- butoxycarbonyl-4-hydroxy- pyrrolidine-2-carboxylic acid and subsequent acidic Boc deprotection |
| 428 | 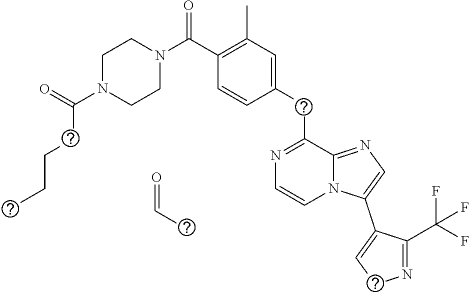 | N-(2- aminoethyl)-4-[2- methyl-4-[[3-[3- (trifluoromethyl)- 1H-pyrazol-4- yl]imidazo[1,2- a]pyrazin-8- yl]amino]benzoyl] piperazine-1- carboxamide formate | 556.5 | 557.3 | in analogy to the procedures for example 12: urea formation of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with N-Boc- ethylenediamine followed by acidic Boc deprotection |
| 429 | 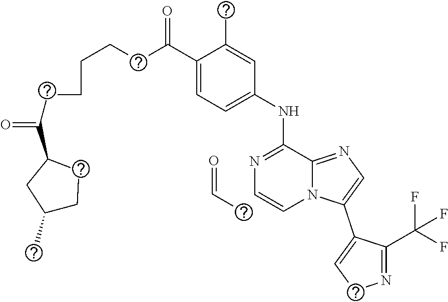 | (2S,4R)-N-[3-[[2- chloro-4-[[3-[3- (trifluoromethyl)- 1H-pyrazol-4- yl]imidazo[1,2- a]pyrazin-8- yl]amino]benzoyl] amino]propyl]-4- hydroxypyrrolidine- 2-carboxamide formate | 592.0 | 592.3 | In analogy to the procedure described for example 1: amide coupling of N-(3-aminopropyl)-2- chloro-4-[[3-[3-(trifluoromethyl)- 1H-pyrazol-4-yl]imidazo[1,2- a]pyrazin-8-yl]amino]benzamide (example 364) with Boc-HYP-OH followed by acidic Boc deprotection |
| 430 | 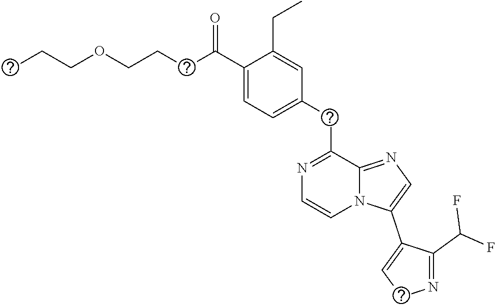 [SMILES: CCCOCCCC(=O)c1ccc(Cc2nccn3c(C4=CCN=C4C(F)F)cnc23)cc1CC] | N-[2-(2- aminoethoxy)ethyl]- 4-[[3-[3- (difluoromethyl)- 1H-pyrazol-4- yl]imidazo[1,2- a]pyrazin-8- yl]amino]-2- ethylbenzamide | 484.5 | 485.3 | In analogy to the procedure described for example 1: acidic Boc deprotection of tert-butyl N- [2-[2-[[4-[[3-[3-(difluoromethyl)- 1H-pyrazol-4-yl]imidazo[1,2- a]pyrazin-8-yl]amino]-2-ethyl- benzoyl]amino]ethoxy]ethyl] carbamate (example 18, step 1) |
| 431 | 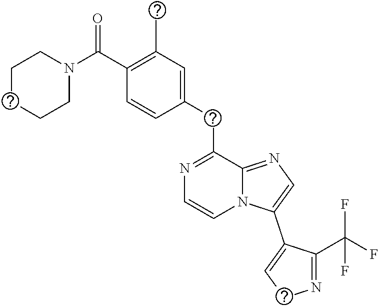 [SMILES: Cc1cc(Cc2nccn3c(C4=CCN=C4C(F)(F)F)cnc23)ccc1C(=O)N1CCCCC1] | [2-chloro-4-[[3- [3- (trifluoromethyl)- 1H-pyrazol-4- yl]imidazo[1,2- a]pyrazin-8- yl]amino]phenyl]- piperazin-1- ylmethanone | 490.9 | 491.2 | example 6, step 5 |
| 432 | 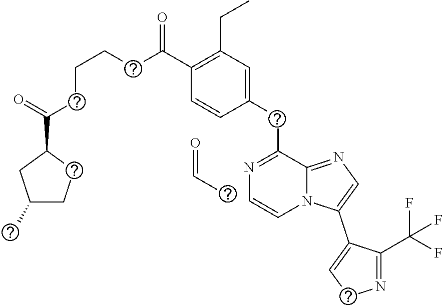 | (2S,4R)-N-[2-[[2- ethyl-4-[[3-[3- (trifluoromethyl)- 1H-pyrazol-4- yl]imidazo[1,2- a]pyrazin-8- yl]amino]benzoyl] amino]ethyl]-4- hydroxypyrrolidine- 2-carboxamide formate | 571.6 | 572.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-Boc-ethylenediamine, subsequent Suzuki reaction with (3-(trifluoromethyl)-1H-pyrazol-4- yl)boronic acid, acidic Boc deprotection, coupling with Boc- HYP-OH and subsequent acidic Boc deprotection |
| 433 | 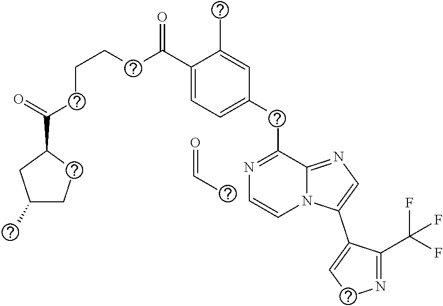 | (2S,4R)-N-[2-[[2- chloro-4-[[3-[3- (trifluoromethyl)- 1H-pyrazol-4- yl]imidazo[1,2- a]pyrazin-8- yl]amino]benzoyl] amino]ethyl]-4- hydroxypyrrolidine- 2-carboxamide formate | 577.9 | 578.3 | in analogy to the procedures for example 1: amide coupling of 2- chloro-4-[(3-iodoimidazo[1,2- a]pyrazin-8-yl)amino]benzoic acid (example 14, step 1) with N-BOC- ethylenediamine, subsequent Suzuki reaction with (3- (trifluoromethyl)-1H-pyrazol-4- yl)boronic acid, acidic Boc deprotection, coupling with Boc- HYP-OH and subsequent acidic Boc deprotection |
| 434 | 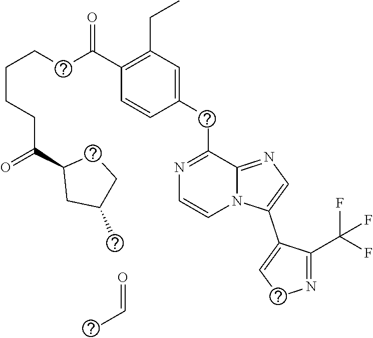 | (2S,4R)-N-[3-[[2- ethyl-4-[[3-[3- (trifluoromethyl)- 1H-pyrazol-4- yl]imidazo[1,2- a]pyrazin-8- yl]amino]benzoyl] amino]propyl]-4- hydroxypyrrolidine- 2-carboxamide formate | 585.6 | 586.4 | in analogy to the procedures for example 1: amide coupling of N- (3-aminopropyl)-2-ethyl-4-[[3-[3- (trifluoromethyl)-1H-pyrazol-4- yl]imidazo[1,2-a]pyrazin-8- yl]amino]benzamide hydrochloride (example 364) with Boc-HYP-OH and subsequent acidic Boc deprotection |
| 435 | 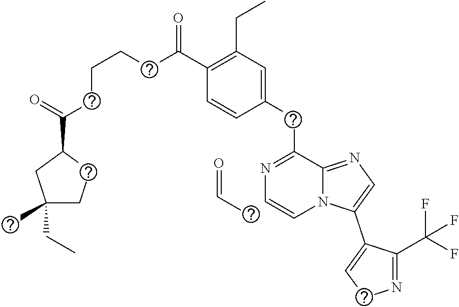 | (2S,4S)-4-ethyl- N-[2-[[2-ethyl-4- [[3-[3- (trifluoromethyl)- 1H-pyrazol-4- yl]imidazo[1,2- a]pyrazin-8- yl]amino]benzoyl] amino]ethyl]-4- hydroxypyrrolidine- 2-carboxamide formate | 599.6 | 600.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-Boc-ethylenediamine, subsequent Suzuki reaction with (3-(trifluoromethyl)-1H-pyrazol-4- yl)boronic acid, acidic Boc deprotection, coupling with (2S,4S)-1-tert-butoxycarbonyl-4- ethyl-4-hydroxy-pyrrolidine-2- carboxylic acid and subsequent acidic Boc deprotection |
| 436 | 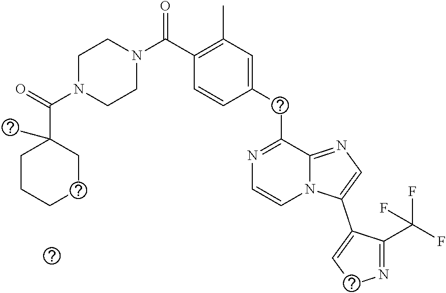 [SMILES: Cc1cc(Cc2nccn3c(C4=CCN=C4C(F)(F)F)cnc23)ccc1C(=O)N1CCN(C(=O)C2(C)CCCCC2)CC1] | [4-(3- hydroxypiperidine- 3- carbonyl)piperazin- 1-yl]-[2-methyl- 4-[[3-[3- (trifluoromethyl)- 1H-pyrazol-4- yl]imidazo[1,2- a]pyrazin-8- yl]amino]phenyl] methanone hydrochloride | 597.5 | 598.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-(tert- butoxycarbonyl)-3- hydroxypiperidine-3-carboxylic acid CAS [1396555-79-5] and acidic Boc deprotection |
| 437 | 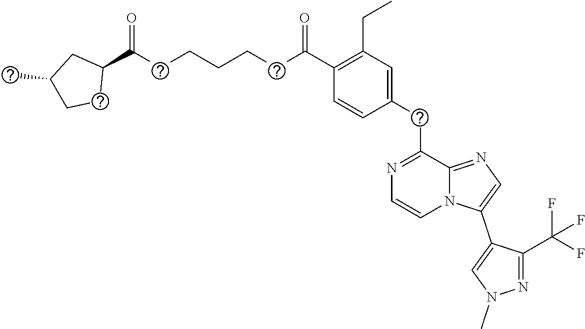 [SMILES: CCc1cc(Cc2nccn3c(-c4cn(C)nc4C(F)(F)F)cnc23)ccc1C(=O)CCCCCC(=O)[C@H]1CC[C@H](C)C1] | (2S,4R)-N-[3-[[2- ethyl-4-[[3-[1- methyl-3- (trifluoromethyl) pyrazol-4- yl]imidazo[1,2- a]pyrazin-8- yl]amino]benzoyl] amino]propyl]-4- hydroxypyrrolidine- 2-carboxamide | 599.6 | 600.4 | In analogy to procedures described for example 13: Suzuki reaction of tert-butyl N-[3-[[2-ethyl-4-[(3- iodoimidazo[1,2-a]pyrazin-8- yl)amino]benzoyl]amino]propyl] carbamate (example 13, step 1) with [1-methyl-3- (trifluoromethyl)pyrazol-4- yl]boronic acid, acidic Boc deprotection, coupling with BOC- HYP-OH and subsequent acidic Boc deprotection |
| 439 | 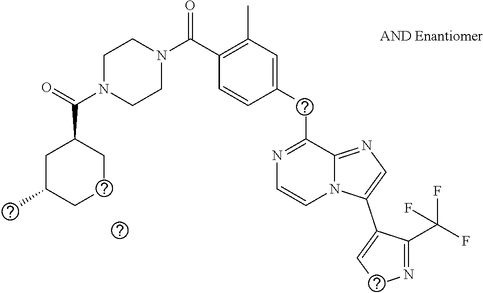 [SMILES: Cc1cc(Cc2nccn3c(C4=CCN=C4C(F)(F)F)cnc23)ccc1C(=O)N1CCN(C(=O)[C@H]2CCC[C@H](C)C2)CC1] | [2-methyl-4-[[3- [3- (trifluoromethyl)- 1H-pyrazol-4- yl]imidazo[1,2- a]pyrazin-8- yl]amino]phenyl]- [4-[rac-(3R,5R)- 5- hydroxypiperidine- 3- carbonyl]piperazin- 1-yl]methanone hydrochloride | 597.6 | 598.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-(tert- butoxycarbonyl)-5- hydroxypiperidine-3-carboxylic acid CAS [1095010-48-2] and acidic Boc deprotection |
| 440 | 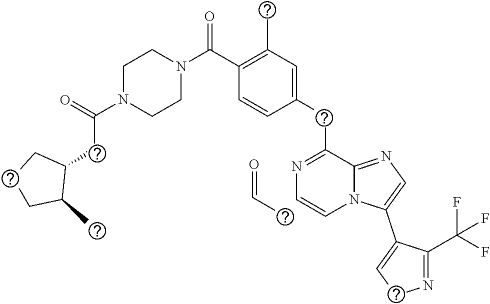 | 4-[2-chloro-4- [[3-[3- (trifluoromethyl)- 1H-pyrazol-4- yl]imidazo[1,2- a]pyrazin-8- yl]amino]benzoyl]- N-[(3S,4S)-4- hydroxypyrrolidin- 3-yl]piperazine- 1-carboxamide formate | 619.0 | 619.2 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl trans-3-amino-4- hydroxy-pyrrolidinecarboxylate followed by acidic Boc deprotection |
| 441 | 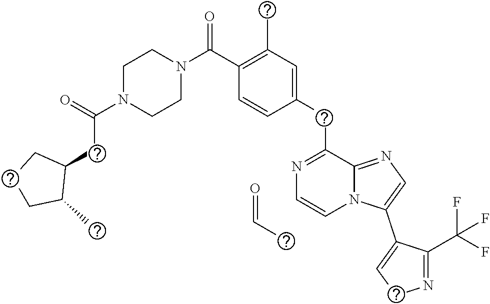 | 4-[2-chloro-4- [[3-[3- (trifluoromethyl)- 1H-pyrazol-4- yl]imidazo[1,2- a]pyrazin-8- yl]amino]benzoyl]- N-[(3R,4R)-4- hydroxypyrrolidin- 3-yl]piperazine- 1-carboxamide formate | 619.0 | 619.1 | in analogy to the procedures for example 12: urea formation with [2-chloro-4-[[3-[3- (trifluoromethyl)-1H-pyrazol-4- yl]imidazo[1,2-a]pyrazin-8- yl]amino]phenyl]-piperazin-1-yl- methanone (example 6, step 5) and tert-butyl (3R,4R)-3-amino-4- hydroxy-pyrrolidine-1-carboxylate followed by acidic Boc deprotection |
| 442 | 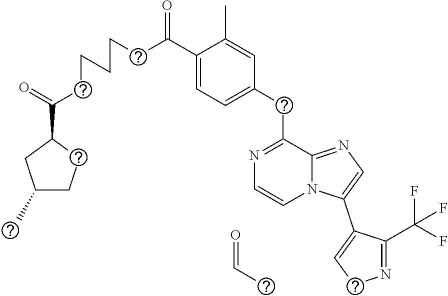 | formic acid; (2S,4R)-4- hydroxy-N-[3-[[2- methyl-4-[[3-[3- (trifluoromethyl)- 1H-pyrazol-4- yl]imidazo[1,2- a]pyrazin-8- yl]amino]benzoyl] amino]propyl] pyrrolidine-2- carboxamide | 571.6 | 572.2 | In analogy to procedures described for example 1: coupling of 4-[(3- iodoimidazo[1,2-a]pyrazin-8- yl)amino]-2-methyl-benzoic acid (example 16, step 1) with N-Boc- 1,3-diaminopropane, subsequent Suzuki reaction with 4-(4,4,5,5- tetramethyl-1,3,2-dioxaborolan-2- yl)-3-(trifluoromethyl)-1H- pyrazole, acidic Boc deprotection, coupling with Boc-HYP-OH followed by acidic Boc deprotection |
| 443 | 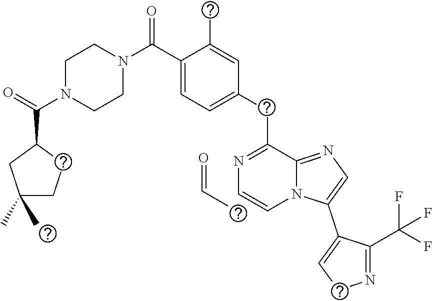 | [2-chloro-4-[[3- [3- (trifluoromethyl)- 1H-pyrazol-4- yl]imidazo[1,2- a]pyrazin-8- yl]amino]phenyl]- [4-[(2S,4S)-4- hydroxy-4- methylpyrrolidine- 2- carbonyl]piperazin- 1-yl]methanone formate | 618.0 | 618.2 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (2S,4S)-1- tert-butoxycarbonyl-4-hydroxy-4- methyl-pyrrolidine-2-carboxylic acid and acidic Boc deprotection |
| 444 | 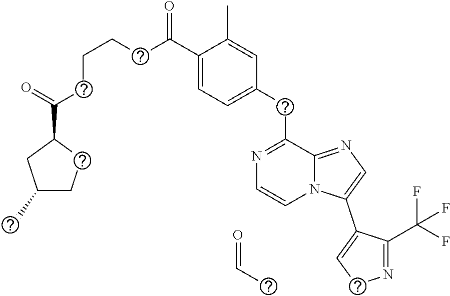 | formic acid; (2S,4R)-4- hydroxy-N-[2-[[2- methyl-4-[[3-[3- (trifluoromethyl)- 1H-pyrazol-4- yl]imidazo[1,2- a]pyrazin-8- yl]amino]benzoyl] amino]ethyl] pyrrolidine-2- carboxamide | 557.5 | 558.2 | in analogy to the procedures for example 1: amide coupling of 4- [(3-iodoimidazo[1,2-a]pyrazin-8- yl)amino]-2-methyl-benzoic acid (example 16, step 1) with N-BOC- 1,2-diaminoethane, Suzuki reaction with 4-(4,4,5,5- tetramethyl-1,3,2-dioxaborolan-2- yl)-3-(trifluoromethyl)-1H- pyrazole, acidic Boc deprotection, coupling with (2S,4R)-1-(tert- butoxycarbonyl)-4- hydroxypyrrolidine-2-carboxylic acid and subsequent acidic Boc deprotection |
| 445 | 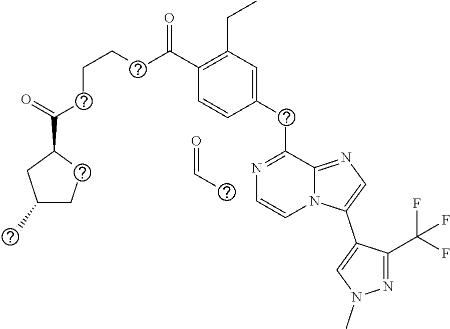 | (2S,4R)-N-[2-[[2- ethyl-4-[[3-[1- methyl-3- (trifluoromethyl) pyrazol-4- yl]imidazo[1,2- a]pyrazin-8- yl]amino]benzoyl] amino]ethyl]-4- hydroxypyrrolidine- 2-carboxamide formate | 585.6 | 586.2 | in analogy to the procedures for example 1: amide coupling of 2- ethyl-4-[(3-iodoimidazo[1,2- a]pyrazin-8-yl)amino]benzoic acid, hydrochloride (example 1, step 2) with N-Boc-1,2-diaminoethane, Suzuki reaction with [1-methyl-3- (trifluoromethyl)pyrazol-4- yl]boronic acid, acidic Boc deprotection, coupling with (2S,4R)-1-(tert-butoxycarbonyl)-4- hydroxypyrrolidine-2-carboxylic acid and subsequent acidic Boc deprotection |
| 446 | 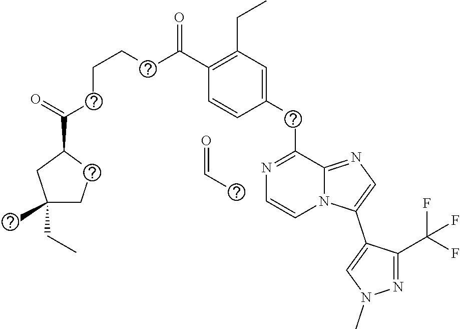 | (2S,4S)-4-ethyl- N-[2-[[2-ethyl-4- [[3-[1-methyl-3- (trifluoromethyl) pyrazol-4- yl]imidazo[1,2- a]pyrazin-8- yl]amino]benzoyl] amino]ethyl]-4- hydroxypyrrolidine- 2-carboxamide formate | 613.6 | 614.3 | in analogy to the procedures for example 1: amide coupling of 2- ethyl-4-[(3-iodoimidazo[1,2- a]pyrazin-8-yl)amino]benzoic acid, hydrochloride (example 1, step 2) with N-Boc-1,2-diaminoethane, Suzuki reaction with [1-methyl-3- (trifluoromethyl)pyrazol-4- yl]boronic acid, acidic Boc deprotection, coupling with (2S,4S)-1-tert-butoxycarbonyl-4- ethyl-4-hydroxy-pyrrolidine-2- carboxylic acid and subsequent acidic Boc deprotection |
| 447 | 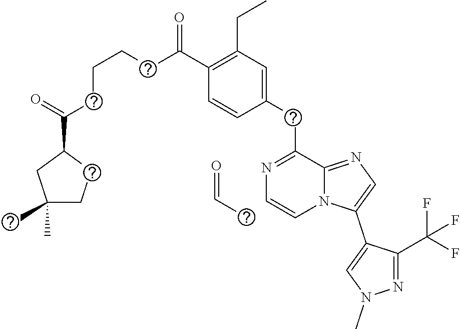 | (2S,4S)-N-[2-[[2- ethyl-4-[[3-[1- methyl-3- (trifluoromethyl) pyrazol-4- yl]imidazo[1,2- a]pyrazin-8- yl]amino]benzoyl] amino]ethyl]-4- hydroxy-4- methylpyrrolidine- 2-carboxamide formate | 599.6 | 600.2 | in analogy to the procedures for example 1: amide coupling of 2- ethyl-4-[(3-iodoimidazo[1,2- a]pyrazin-8-yl)amino]benzoic acid, hydrochloride (example 1, step 2) with N-Boc-1,2-diaminoethane, Suzuki reaction with [1-methyl-3- (trifluoromethyl)pyrazol-4- yl]boronic acid, acidic Boc deprotection, coupling with (2S,4S)-1-tert-butoxycarbonyl-4- hydroxy-4-methyl-pyrrolidine-2- carboxylic acid and subsequent acidic Boc deprotection |
| 448 | 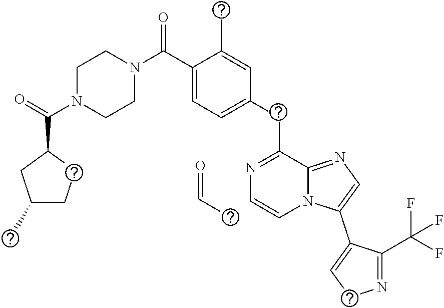 | [2-chloro-4-[[3- [3- (trifluoromethyl)- 1H-pyrazol-4- yl]imidazo[1,2- a]pyrazin-8- yl]amino]phenyl]- [4-[(2S,4R)-4- hydroxypyrrolidine- 2- carbonyl]piperazin- 1-yl]methanone formate | 604.0 | 604.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with Boc- HYP-OH and acidic Boc deprotection |
| 449 | 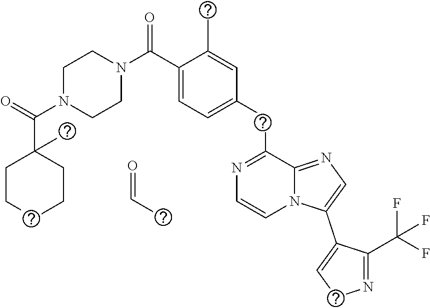 | [2-chloro-4-[[3- [3- (trifluoromethyl)- 1H-pyrazol-4- yl]imidazo[1,2- a]pyrazin-8- yl]amino]phenyl]- [4-(4- hydroxypiperidine- 4- carbonyl)piperazin- 1-yl]methanone formate | 618.0 | 618.1 | in analogy to the procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with 1-tert- butoxycarbonyl-4-hydroxy- piperidine-4-carboxylic acid and acidic Boc deprotection |
| 450 | 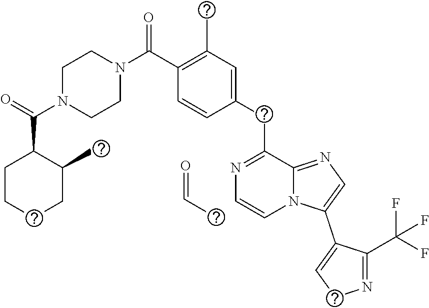 | [2-chloro-4-[[3- [3- (trifluoromethyl)- 1H-pyrazol-4- yl]imidazo[1,2- a]pyrazin-8- yl]amino]phenyl]- [4-[(3R,4R)-3- hydroxypiperidine- 4- carbonyl]piperazin- 1-yl]methanone formate | 618.0 | 618.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (3R,4R)- 1-tert-butoxycarbonyl-3-hydroxy- piperidine-4-carboxylic acid and acidic Boc deprotection |
| 451 | 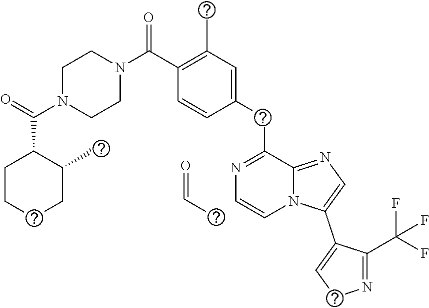 | [2-chloro-4-[[3- [3- (trifluoromethyl)- 1H-pyrazol-4- yl]imidazo[1,2- a]pyrazin-8- yl]amino]phenyl]- [4-[(3S,4S)-3- hydroxypiperidine- 4- carbonyl]piperazin- 1-yl]methanone formate | 618.0 | 618.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (3S,4S)-1- tert-butoxycarbonyl-3-hydroxy- piperidine-4-carboxylic acid and acidic Boc deprotection |
| 452 | 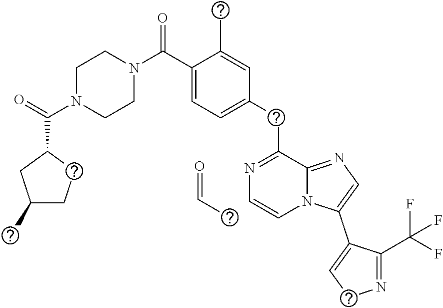 | [2-chloro-4-[[3- [3- (trifluoromethyl)- 1H-pyrazol-4- yl]imidazo[1,2- a]pyrazin-8- yl]amino]phenyl]- [4-[(2R,4S)-4- hydroxypyrrolidine- 2- carbonyl]piperazin- 1-yl]methanone formate | 604.0 | 604.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (2R,4S)- 1-tert-butoxycarbonyl-4-hydroxy- pyrrolidine-2-carboxylic acid and acidic Boc deprotection |
| 453 | 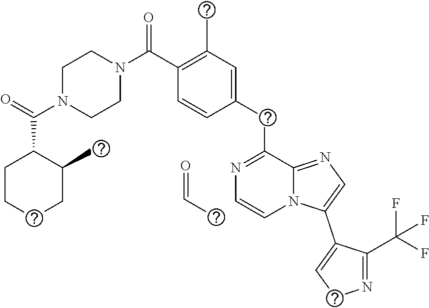 | [2-chloro-4-[[3- [3- (trifluoromethyl)- 1H-pyrazol-4- yl]imidazo[1,2- a]pyrazin-8- yl]amino]phenyl]- [4-[(3R,4S)-3- hydroxypiperidine- 4- carbonyl]piperazin- 1-yl]methanone formate | 618.0 | 618.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (3R,4S)- 1-tert-butoxycarbonyl-3-hydroxy- piperidine-4-carboxylic acid and acidic Boc deprotection |
| 454 | 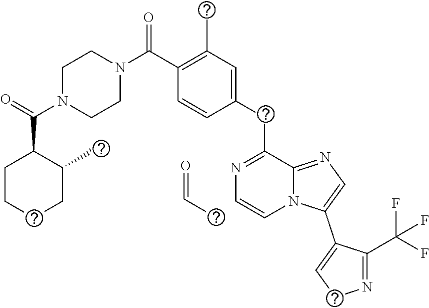 | [2-chloro-4-[[3- [3- (trifluoromethyl)- 1H-pyrazol-4- yl]imidazo[1,2- a]pyrazin-8- yl]amino]phenyl]- [4-[(3S,4R)-3- hydroxypiperidine- 4- carbonyl]piperazin- 1-yl]methanone formate | 618.0 | 618.1 | in analogy to procedures for example 6: amide coupling of [2- chloro-4-[[3-[3-(trifluoromethyl)- 1H-pyrazol-4-yl]imidazo[1,2- a]pyrazin-8-yl]amino]phenyl]- piperazin-1-yl-methanone (example 6, step 5) with (3S,4R)- 1-tert-butoxycarbonyl-3-hydroxy- piperidine-4-carboxylic acid and acidic Boc deprotection |
| 455 | 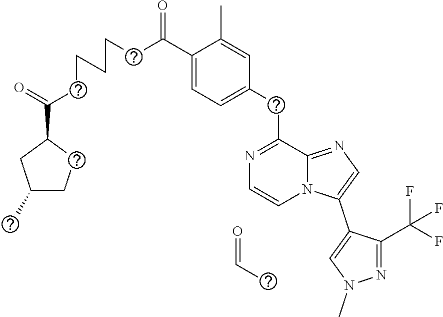 | formic acid; (2S,4R)-4- hydroxy-N-[3-[[2- methyl-4-[[3-[1- methyl-3- (trifluoromethyl) pyrazol-4- yl]imidazo[1,2- a]pyrazin-8- yl]amino]benzoyl] amino]propyl] pyrrolidine-2- carboxamide | 585.6 | 586.2 | in analogy to the procedures for example 1: amide coupling of 4- [(3-iodoimidazo[1,2-a]pyrazin-8- yl)amino]-2-methyl-benzoic acid (example 16, step 1) with N-Boc- 1,3-diaminopropane, subsequent Suzuki reaction with [1-methyl-3- (trifluoromethyl)pyrazol-4- yl]boronic acid, acidic Boc deprotection, coupling with Boc- HYP-OH and subsequent acidic Boc deprotection |
| 456 | 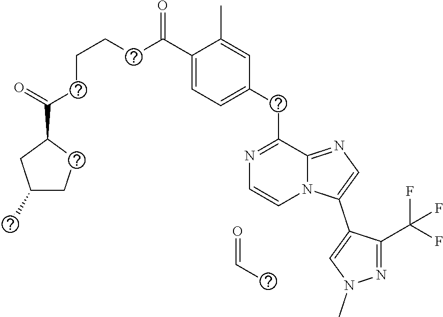 | formic acid; (2S,4R)-4- hydroxy-N-[2-[[2- methyl-4-[[3-[1- methyl-3- (trifluoromethyl) pyrazol-4- yl]imidazo[1,2- a]pyrazin-8- yl]amino]benzoyl] amino]ethyl] pyrrolidine-2- carboxamide | 571.6 | 572.2 | in analogy to the procedures for example 1: amide coupling of 4- [(3-iodoimidazo[1,2-a]pyrazin-8- yl)amino]-2-methyl-benzoic acid (example 16, step 1) with N-Boc- 1,2-diaminoethane, Suzuki reaction with [1-methyl-3- (trifluoromethyl)pyrazol-4- yl]boronic acid, acidic Boc deprotection, coupling with Boc- HYP-OH and subsequent acidic Boc deprotection |
| 457 | 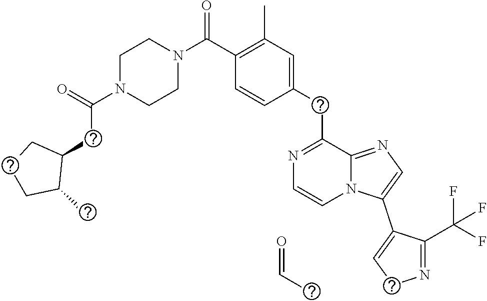 | formic acid, N- [(3R,4R)-4- hydroxypyrrolidin- 3-yl]-4-[2- methyl-4-[[3-[3- (trifluoromethyl)- 1H-pyrazol-4- yl]imidazo[1,2- a]pyrazin-8- yl]amino]benzoyl] piperazine-1- carboxamide | 598.6 | 599.3 | In analogy to procedures described for example 12: urea formation of [2-methyl-4-[[3-[3- (trifluoromethyl)-1H-pyrazol-4- yl]imidazo[1,2-a]pyrazin-8- yl]amino]phenyl]-piperazin-1-yl- methanone (example 17, step 3) with tert-butyl (3R,4R)-3-amino-4- hydroxy-pyrrolidine-1-carboxylate and acidic Boc deprotection |
| 458 | 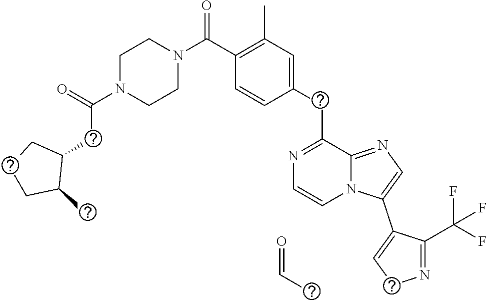 | formic acid, N- [(3S,4S)-4- hydroxyppyrrolidin- 3-yl]-4-[2- methyl-4-[[3-[3- (trifluoromethyl)- 1H-pyrazol-4- yl]imidazo[1,2- a]pyrazin-8- yl]amino]benzoyl] piperazine-1- carboxamide | 598.6 | 599.3 | In analogy to procedures described for example 12: urea formation of [2-methyl-4-[[3-[3- (trifluoromethyl)-1H-pyrazol-4- yl]imidazo[1,2-a]pyrazin-8- yl]amino]phenyl]-piperazin-1-yl- methanone (example 17, step 3) with tert-butyl (3S,4S)-3-amino-4- hydroxy-pyrrolidine-1-carboxylate and acidic Boc deprotection |
| 459 | 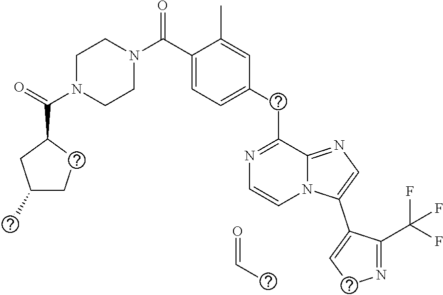 | formic acid; [4- [(2S,4R)-4- hydroxypyrrolidine- 2- carbonyl)piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 583.6 | 584.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with Boc- HYP-OH and acidic Boc deprotection |
| 460 | 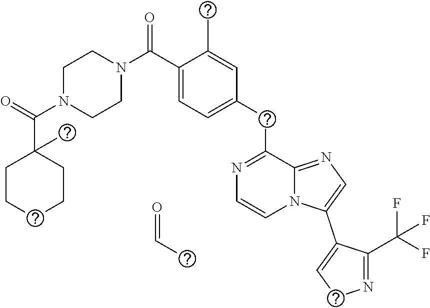 | formic acid; [4-(4- hydroxypiperidine- 4- carbonyl)piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 597.6 | 598.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-tert- butoxycarbonyl-4-hydroxy- piperidine-4-carboxylic acid and acidic Boc deprotection |
| 461 | 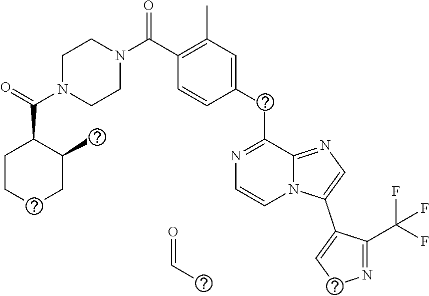 | formic acid; [4- [(3R,4R)-3- hydroxypiperidine- 4- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 597.6 | 598.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (3R,4R)- 1-tert-butoxycarbonyl-3-hydroxy- piperidine-4-carboxylic acid and acidic Boc deprotection |
| 462 | 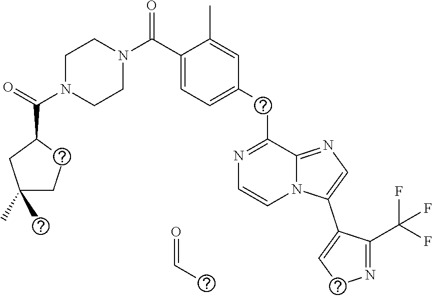 | formic acid; [4- [(2S,4S)-4- hydroxy-4- methylpyrrolidine- 2- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 597.6 | 598.2 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (2S,4S)- 1-tert-butoxycarbonyl-3-hydroxy- 4-methyl-pyrrolidine-2-carboxylic acid and acidic Boc deprotection |
| 463 | 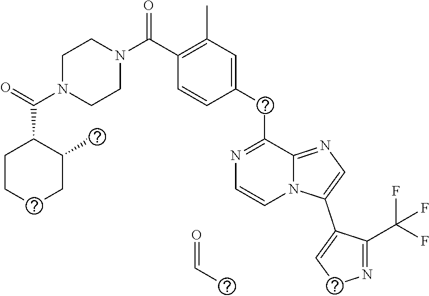 | formic acid; [4- [(3S,4S)-3- hydroxypiperidine- 4- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 597.6 | 598.2 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (3S,4S)- 1-tert-butoxycarbonyl-3-hydroxy- piperidine-4-carboxylic acid and acidic Boc deprotection |
| 464 | 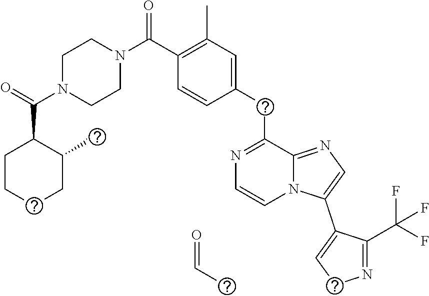 | formic acid; [4- [(3S,4R)-3- hydroxypiperidine- 4- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 597.6 | 598.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (3S,4R)- 1-tert-butoxycarbonyl-3-hydroxy- piperidine-4-carboxylic acid and acidic Boc deprotection |
| 465 | 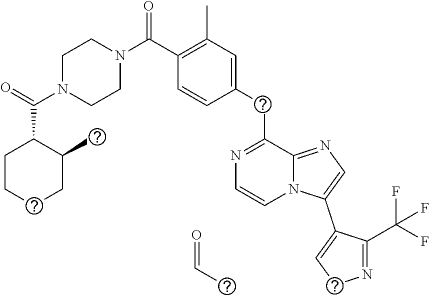 | formic acid; [4- [(3R,4S)-3- hydroxypiperidine- 4- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 597.6 | 598.2 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (3R,4S)- 1-tert-butoxycarbonyl-3-hydroxy- piperidine-4-carboxylic acid and acidic Boc deprotection |
| 466 | 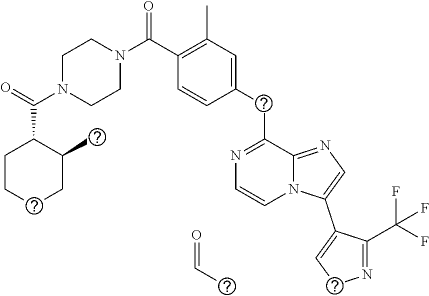 | formic acid; [4- [(2R,4S)-4- hydroxypyrrolidine- 2- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone | 583.6 | 584.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (2R,4S)- 1-tert-butoxycarbonyl-4-hydroxy- pyrrolidine-2-carboxylic acid and acidic Boc deprotection |
| 467 | 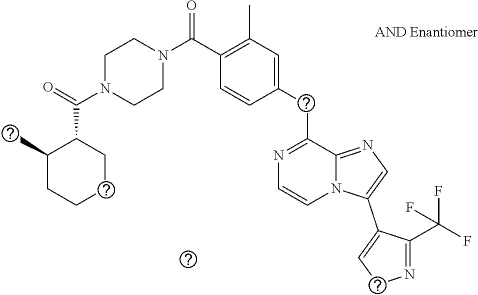 [SMILES: Cc1cc(Cc2nccn3c(C4=CCN=C4C(F)(F)F)cnc23)ccc1C(=O)N1CCN(C(=O)[C@@H]2CCCC[C@H]2C)CC1] | [2-methyl-4-[[3- [3- (trifluoromethyl)- 1H-pyrazol-4- yl]imidazo[1,2- a]pyrazin-8- yl]amino]phenyl]- [4-[rac-(3R,4R)- 4- hydroxypiperidine- 3- carbonyl]piperazin- 1-yl]methanone hydrochloride | 597.6 | 598.2 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-(tert- butoxycarbonyl-4- hydroxypiperidine-3-carboxylic acid CAS [220182-20-7] and acidic Boc deprotection |
| 468 | 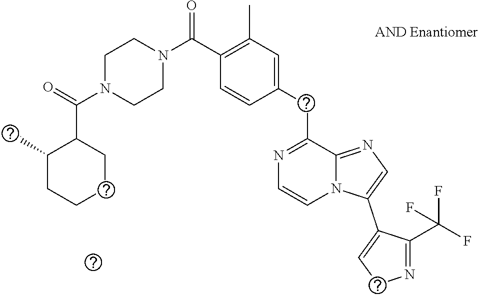 [SMILES: Cc1cc(Cc2nccn3c(C4=CCN=C4C(F)(F)F)cnc23)ccc1C(=O)N1CCN(C(=O)C2CCCC[C@@H]2C)CC1] | [2-methyl-4-[[3- [3- (trifluoromethyl)- 1H-pyrazol-4- yl]imidazo[1,2- a]pyrazin-8- yl]amino]phenyl]- [4-[rac-(3R,4S)-4- hydroxypiperidine- 3- carbonyl]piperazin- 1-yl]methanone hydrochloride | 597.6 | 598.2 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with 1-(tert- butoxycarbonyl)-4- hydroxypiperidine-3-carboxylic acid CAS [220182-20-7] and acidic Boc deprotection |
| 469 | 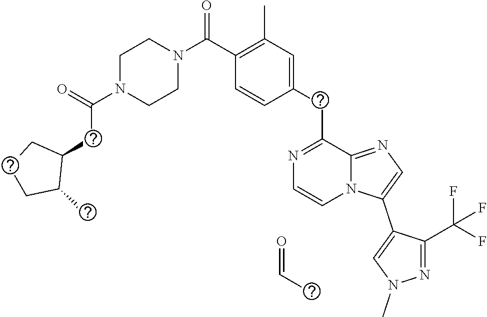 | formic acid; N- [(3R,4R)-4- hydroxypyrrolidin- 3-yl]-4-[2- methyl-4-[[3-[1- methyl-3- (trifluoromethyl) pyrazol-4- yl]imidazo[1,2- a]pyrazin-8- yl]amino]benzoyl] piperazine-1- carboxamide | 612.6 | 613.4 | In analogy to procedures described for example 1/12: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, urea formation with tert-butyl (3R,4R)-3-amino-4- hydroxy-pyrrolidine-1-carboxylate and subsequent acidic Boc deprotection |
| 470 | 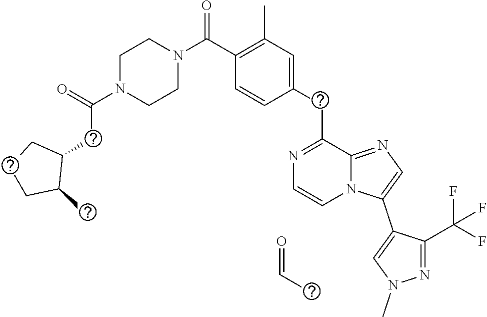 | formic acid; N- [(3S,4S)-4- hydroxypyrrolidin- 3-yl]-4-[2- methyl-4-[[3-[1- methyl-3- (trifluoromethyl) pyrazol-4- yl]imidazo[1,2- a]pyrazin-8- yl]amino]benzoyl] piperazine-1- carboxamide | 612.6 | 613.4 | In analogy to procedures described for example 1/12: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, urea formation with tert-butyl trans-3-amino-4- hydroxy-1-pyrrolidinecarboxylate and subsequent acidic Boc deprotection |
| 471 | 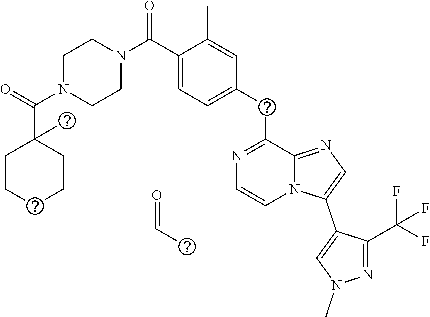 | formic acid; [4-(4- hydroxypiperidine- 4- carbonyl)piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 611.6 | 612.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with 1-tert- butoxycarbonyl-4-hydroxy- piperidine-4-carboxylic acid and subsequent acidic Boc deprotection |
| 472 | 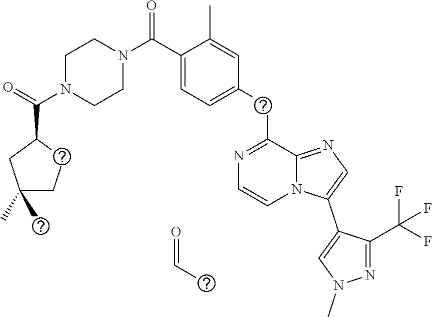 | formic acid; [4- [(2S,4S)-4- hydroxy-4- methylpyrrolidine- 2- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 611.6 | 612.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with (2S,4S)-1-tert-butoxycarbonyl-4- hydroxy-4-methyl-pyrrolidine-2- carboxylic acid and subsequent acidic Boc deprotection |
| 473 | 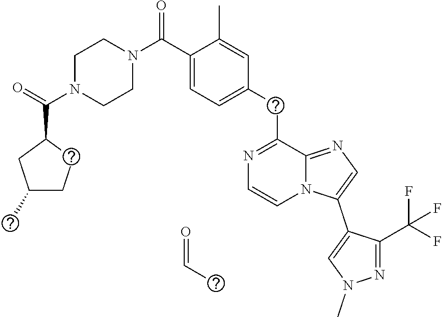 | formic acid; [4- [(2S,4R)-4- hydroxypyrrolidine- 2- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 597.6 | 598.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with Boc- HYP-OH and subsequent acidic Boc deprotection |
| 474 | 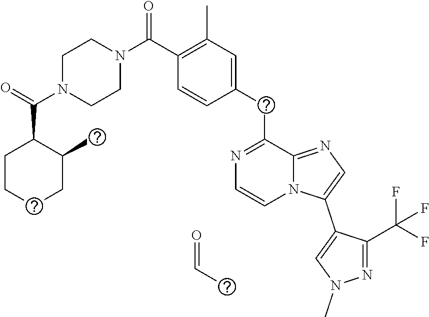 | formic acid; [4- [(3R,4R)-3- hydroxypiperidine- 4- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 611.6 | 612.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with (3R,4R)-1-tert-butoxycarbonyl-3- hydroxy-piperidine-4-carboxylic acid and subsequent acidic Boc deprotection |
| 475 | 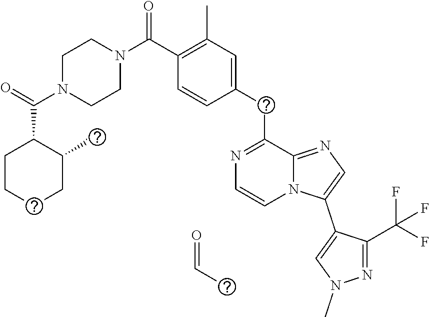 | formic acid; [4- [(3S,4S)-3- hydroxypiperidine- 4- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 611.6 | 612.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with (3S,4S)-1-tert-butoxycarbonyl-3- hydroxy-piperidine-4-carboxylic acid and subsequent acidic Boc deprotection |
| 476 | 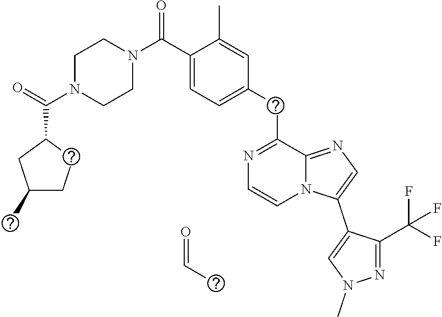 | formic acid; [4- [(2R,4S)-4- hydroxypyrrolidine- 2- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 597.6 | 598.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with (2R,4S)-1-tert-butoxycarbonyl-4- hydroxy-pyrrolidine-2-carboxylic acid and subsequent acidic Boc deprotection |
| 477 | 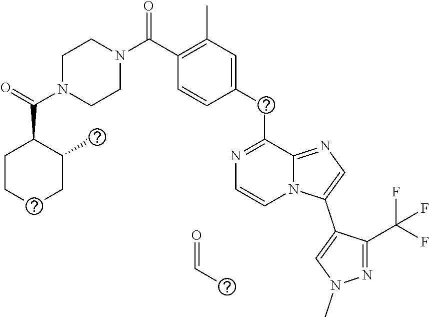 | formic acid; [4- [(3S,4R)-3- hydroxypiperidine- 4- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 611.6 | 612.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with (3S,4R)-1-tert-butoxycarbonyl-3- hydroxy-piperidine-4-carboxylic acid and subsequent acidic Boc deprotection |
| 478 | 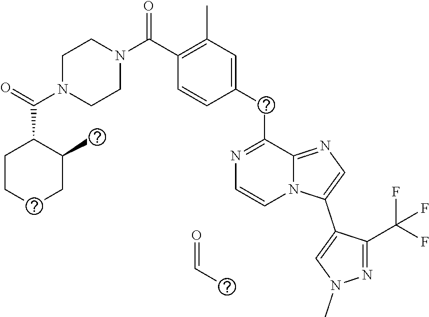 | formic acid; [4- [(3R,4S)-3- hydroxypiperidine- 4- carbonyl]piperazin- 1-yl]-[2-methyl- 4-[[3-[1-methyl-3- (trifluoromethyl) pyrazol-4- yl]imidazo[1,2- a]pyrazin-8- yl]amino]phenyl] methanone | 611.6 | 612.4 | In analogy to procedures described for example 1: Suzuki reaction of tert-butyl 4-[4-[(3- iodoimidazo[1,2-a]pyrazin-8- yl)amino]-2-methyl- benzoyl]piperazine-1-carboxylate (example 17, step 1) with [1- methyl-3-(trifluoromethyl)pyrazol- 4-yl]boronic acid, acidic Boc deprotection, coupling with (3R,4S)-1-tert-butoxycarbonyl-3- hydroxy-piperidine-4-carboxylic acid and subsequent acidic Boc deprotection |
| 479 | 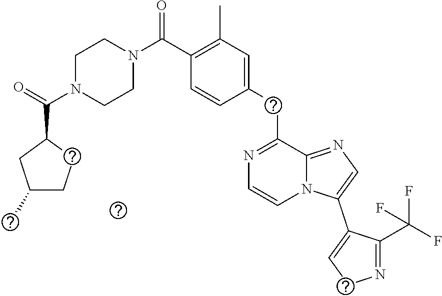 [SMILES: Cc1cc(Cc2nccn3c(C4=CCN=C4C(F)(F)F)cnc23)ccc1C(=O)N1CCN(C(=O)[C@H]2CC[C@H](C)C2)CC1] | [4-[(2S,4R)-4- hydroxypyrrolidine- 2- carbonyl]piperazin- 1-yl]-[2-methyl- 4-[[3-[3- (trifluoromethyl)- 1H-pyrazol-4- yl]imidazo[1,2- a]pyrazin-8- yl]amino]phenyl] methanone hydrochloride | 583.6 | 584.3 | In analogy to procedures described for example 17: coupling of [2- methyl-4-[[3-[3-(trifluoromethyl)- 1H-pyrazol-4-yl]imidazo[1,2- a]pyrazin-8-yl]amino]phenyl]- piperazin-1-yl-methanone (example 17, step 3) with (2S,4R)- 1-(tert-butoxycarbonyl)-4- hydroxypyrrolidine-2-carboxylic acid CAS: [13726-69-7] and acidic Boc deprotection |
| 480 | 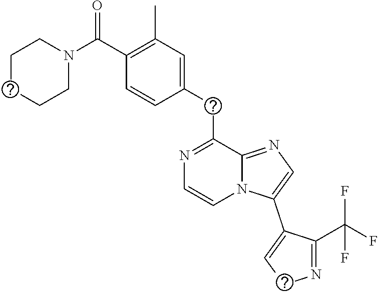 [SMILES: Cc1cc(Cc2nccn3c(C4=CCN=C4C(F)(F)F)cnc23)ccc1C(=O)N1CCCCC1] | [2-methyl-4-[[3- [3- (trifluoromethyl)- 1H-pyrazol-4- yl]imidazo[1,2- a]pyrazin-8- yl]amino]phenyl]- piperazin-1- ylmethanone | 470.5 | 471.3 | example 17, step 3 |

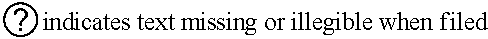

### Example 481

### 2-(4-(8-((4-(4-((1R,5 S,6r)-3-azabicyclo[3.1.0]hexane-6-carbonyl)piperazine-1-carbonyl)-3-chlorophenyl)amino)imidazo[1,2-a]pyrazin-3-yl)-3-(trifluoromethyl)-1H-pyrazol-1-yl)acetonitrile formate

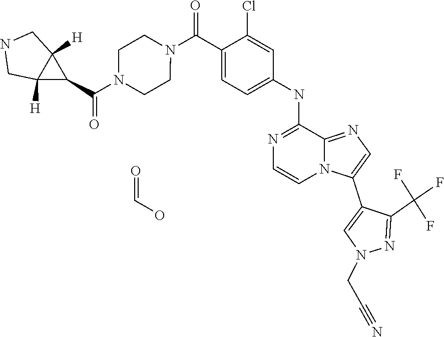 [SMILES: N#CCn1cc(-c2cnc3c(Nc4ccc(C(=O)N5CCN(C(=O)[C@H]6[C@@H]7CNC[C@@H]76)CC5)c(Cl)c4)nccn23)c(C(F)(F)F)n1 ; O=CO]

Step 1:

### tert-butyl (1R,5S,6r)-6-(4-(2-chloro-4-((3-(3-(trifluoromethyl)-1H-pyrazol-4-yl)imidazo[1,2-a]pyrazin-8-yl)amino)benzoyl)piperazine-1-carbonyl)-3-azabicyclo[3.1.0]hexane-3-carboxylate

In analogy to the procedure described for example 3, step 5 the title compound was prepared from [2-chloro-4-[[3-[3-(trifluoromethyl)-1H-pyrazol-4-yl]imidazo[1,2-a]pyrazin-8-yl]amino]phenyl]-piperazin-1-yl-methanone (example 6, step 5) and (1R,5S,6r)-3-(tert-butoxycarbonyl)-3-azabicyclo[3.1.0]hexane-6-carboxylic acid CAS [927679-54-7]. MS(m/e): 700.4 (M+H).

Step 2:

### tert-butyl (1R,5S,6r)-6-(4-(2-chloro-4-((3-(1-(cyanomethyl)-3-(trifluoromethyl)-1H-pyrazol-4-yl)imidazo[1,2-a]pyrazin-8-yl)amino)benzoyl)piperazine-1-carbonyl)-3-azabicyclo[3.1.0]hexane-3-carboxylate

To a microwave vial was added tert-butyl (1R,5S,6r)-6-(4-(2-chloro-4-((3-(3-(trifluoromethyl)-1H-pyrazol-4-yl)imidazo[1,2-a]pyrazin-8-yl)amino)benzoyl)piperazine-1-carbonyl)-3-azabicyclo[3.1.0]hexane-3-carboxylate (40 mg, 57.1 μmol), 2-bromoacetonitrile CAS [590-17-0](13.7 mg, 114 μmol) and sodium bicarbonate CAS [144-55-8] (14.4 mg, 171 μmol) in DMF (0.5 ml). The vial was capped and heated in the microwave at 120° C. for 15 min. The crude reaction mixture was purified by preparative HPLC. The relevant fractions were combined and the solvent was removed by freezing at −78° C. and subsequent lyophilization overnight to yield the title compound as a white lyoph solid (33 mg, 42.4 μmol, 74.2% yield). MS(m/e): 739.4 (M+H).

Step 3:

### 2-(4-(8-((4-(4-((1R,5S,6r)-3-azabicyclo[3.1.0]hexane-6-carbonyl)piperazine-1-carbonyl)-3-chlorophenyl)amino)imidazo[1,2-a]pyrazin-3-yl)-3-(trifluoromethyl)-1H-pyrazol-1-yl)acetonitrile formate

tert-butyl (1R,5S,6r)-6-(4-(2-chloro-4-((3-(1-(cyanomethyl)-3-(trifluoromethyl)-1H-pyrazol-4-yl)imidazo[1,2-a]pyrazin-8-yl)amino)benzoyl)piperazine-1-carbonyl)-3-azabicyclo[3.1.0]hexane-3-carboxylate (33 mg, 44.6 μmol) was combined with DCM (2 ml) to give a colorless solution. TFA CAS [76-05-1] (102 mg, 68.8 μl, 893 μmol) was added and the reaction mixture was stirred at 20° C. overnight. The reaction mixture was quenched by addition of DCM/MeOH/NH₃ (95/5/1) until pH was basic. The solvent was then fully evaporated. The crude was purified by preparative HPLC (1% to 100% acetonitrile in water (+0.1% HCOOH)). The relevant fractions were combined and the solvent was removed by freezing at −78° C. and subsequent lyophilization overnight to yield the title compound as a white lyoph solid (26 mg, 32.6 μmol, 73.1% yield). MS(m/e): 683.4 (M+HCOO)—.

### Example 482

### N-(3-((1s,3s)-3-aminocyclobutane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide formate

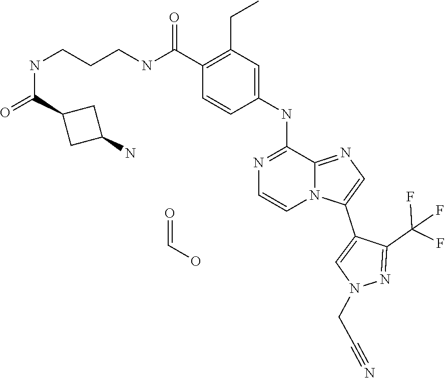 [SMILES: CCc1cc(Nc2nccn3c(-c4cn(CC#N)nc4C(F)(F)F)cnc23)ccc1C(=O)NCCCNC(=O)[C@H]1C[C@@H](N)C1 ; O=CO]

Step 1:

### tert-butyl (3-(2-ethyl-4-((3-(3-(trifluoromethyl)-1H-pyrazol-4-yl)imidazo[1,2-a]pyrazin-8-yl)amino)benzamido)propyl)carbamate

In analogy to the amide coupling procedure described for the synthesis of tert-butyl N-[2-[[2-ethyl-4-[[3-[3-(trifluoromethyl)-1H-pyrazol-4-yl]imidazo[1,2-a]pyrazin-8-yl]amino]benzoyl]amino]ethyl]carbamate (example 3, step 3) the title compound was prepared from 2-ethyl-4-[[3-[3-(trifluoromethyl)-1H-pyrazol-4-yl]imidazo[1,2-a]pyrazin-8-yl]amino]benzoic acid, hydrochloride (example 3, step 2) and tert-butyl (3-aminopropyl)carbamate CAS [75178-96-0]. MS(m/e): 573.3 (M+H).

Step 2:

### N-(3-aminopropyl)-2-ethyl-4-((3-(3-(trifluoromethyl)-1H-pyrazol-4-yl)imidazo[1,2-a]pyrazin-8-yl)amino)benzamide

tert-butyl (3-(2-ethyl-4-((3-(3-(trifluoromethyl)-1H-pyrazol-4-yl)imidazo[1,2-a]pyrazin-8-yl)amino)benzamido)propyl)carbamate (1.596 g, 2.79 mmol) was combined with MeOH (30 ml) to give a brown solution. Hydrochloric acid solution (4M in dioxane) CAS [7647-01-0] (6.97 ml, 27.9 mmol) was added and the reaction mixture was stirred at 20° C. overnight. The brown solution was neutralized with triethylamine CAS [121-44-8] (3.38 g, 4.66 ml, 33.4 mmol). The crude solution was purified by reversed phase flash chromatography (C18 RediSep Gold, 5% to 100% acetonitrile in water (+0.1% aq. NH₄OH)). The relevant fractions were combined and the solvent was removed by freezing at −78° C. and subsequent lyophilization overnight to yield the title compound as a light brown lyoph solid (1015 mg, 2.08 mmol, 74.8% yield). MS(m/e): 471.3 (M−H)−.

Step 3:

### tert-butyl ((1s,3s)-3-((3-(2-ethyl-4-((3-(3-(trifluoromethyl)-1H-pyrazol-4-yl)imidazo[1,2-a]pyrazin-8-yl)amino)benzamido)propyl)carbamoyl)cyclobutyl)carbamate

In analogy to the procedure described for example 3, step 5 the title compound was prepared from N-(3-aminopropyl)-2-ethyl-4-((3-(3-(trifluoromethyl)-1H-pyrazol-4-yl)imidazo[1,2-a]pyrazin-8-yl)amino)benzamide and (1s,3s)-3-((tert-butoxycarbonyl)amino)cyclobutane-1-carboxylic acid CAS [1008773-79-2]. MS(m/e): 668.5 (M+H).

Step 4:

### tert-butyl ((1s,3s)-3-((3-(4-((3-(1-(cyanomethyl)-3-(trifluoromethyl)-1H-pyrazol-4-yl)imidazo[1,2-a]pyrazin-8-yl)amino)-2-ethylbenzamido)propyl)carbamoyl)cyclobutyl)carbamate

In analogy to the procedure described for example 481, step 2 the title compound was prepared from tert-butyl ((1s,3s)-3-((3-(2-ethyl-4-((3-(3-(trifluoromethyl)-1H-pyrazol-4-yl)imidazo[1,2-a]pyrazin-8-yl)amino)benzamido)propyl)carbamoyl)cyclobutyl)carbamate. MS(m/e): 707.6 (M−H)−.

Step 5:

### N-(3-((1s,3s)-3-aminocyclobutane-1-carboxamido)propyl)-4-((3-(1-(cyanomethyl)-3-(trifluoromethyl)-1H-pyrazol-4-yl)imidazo[1,2-a]pyrazin-8-yl)amino)-2-ethylbenzamide formate

In analogy to the procedure described for example 481, step 3 the title compound was prepared from -tert-butyl ((1s,3s)-3-((3-(4-((3-(1-(cyanomethyl)-3-(trifluoromethyl)-1H-pyrazol-4-yl)imidazo[1,2-a]pyrazin-8-yl)amino)-2-ethylbenzamido)propyl)carbamoyl)cyclobutyl)carbamate. MS(m/e): 653.5 (M+HCOO)—.

The following examples were synthesized in analogy to procedures described before. The reaction sequence and staring materials are recorded in the table 2.

TABLE 2

| | | | | MW | |
|---|---|---|---|---|---|
| | | Systematic | Parent | (M + H) | Reaction Sequence and |
| No | Stucture | Name | MW | found | Starting Materials |
| 483 | 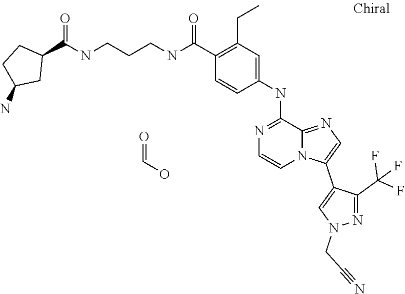 [SMILES: CCc1cc(Nc2nccn3c(-c4cn(CC#N)nc4C(F)(F)F)cnc23)ccc1C(=O)NCCCNC(=O)[C@@H]1CC[C@H](N)C1 ; O=CO] | N-(3-((1R,3S)-3- (l2- azaneyl) cyclopentane-1- carboxamido) propyl)-4-((3-(1- (cyanomethyl)-3- (trifluoromethyl)- 1H-pyrazol-4- yl)imidazo[1,2- a]pyrazin-8- yl)amino)-2- ethylbenzamide formate | 622.7 | 667.5 (M + HCOO)− | in analogy to the procedure for example 482: amide coupling of N-(3- aminopropyl)-2-ethyl-4-((3- (3-(trifluoromethyl)-1H- pyrazol-4-yl)imidazo[1,2- a]pyrazin-8- yl)amino)benzamide (example 482, step 2) and (1R,3S)-3-((tert- butoxycarbonyl)amino) cyclopentane-1-carboxylic acid [CAS#161660-94-2], alkylation with 2- bromoacetonitrile [CAS#590- 17-0] and subsequent acidic Boc deprotection |
| 484 | 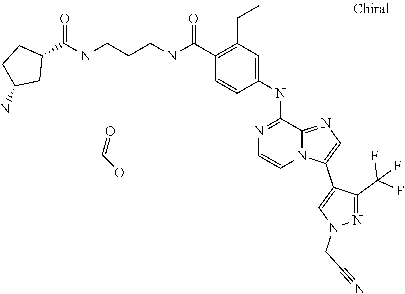 [SMILES: CCc1cc(Nc2nccn3c(-c4cn(CC#N)nc4C(F)(F)F)cnc23)ccc1C(=O)NCCCNC(=O)[C@H]1CC[C@@H](N)C1 ; O=CO] | N-(3-((1S,3R)-3- aminocyclopentane- 1-carboxamido) propyl)-4-((3-(1- (cyanomethyl)-3- (trifluoromethyl)- 1H-pyrazol-4- yl)imidazo[1,2- a]pyrazin-8- yl)amino)-2- ethylbenzamide formate | 622.7 | 667.5 (M + HCOO)− | in analogy to the procedure for example 482: amide coupling of N-(3- aminopropyl)-2 -ethyl-4-((3- (3-(trifluoromethyl)-1H- pyrazol-4-yl)imidazo[1,2- a]pyrazin-8- yl)amino)benzamide (example 482, step 2) and (1S,3R)-3-((tert- butoxy carbonyl)amino) cyclopentane-1-carboxylic acid [CAS#261165-05-3], alkylation with 2- bromoacetonitrile [CAS#590- 17-0] and subsequent acidic Boc deprotection |
| 485 | 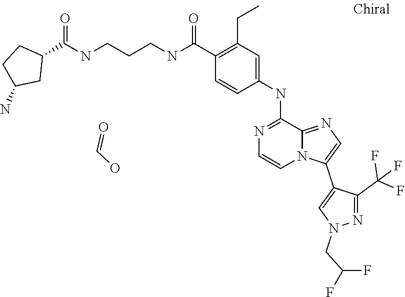 [SMILES: CCc1cc(Nc2nccn3c(-c4cn(CC(F)F)nc4C(F)(F)F)cnc23)ccc1C(=O)NCCCNC(=O)[C@H]1CC[C@@H](N)C1 ; O=CO] | N-(3-((1S,3R)-3- aminocyclopentane- 1-carboxamido) propsl)-4-((3-(1- (2,2- difluoroethyl)-3- (trifluoromethyl)- 1H-pyrazol-4- yl)imidazo[1,2- a]pyrazin-8- yl)amino)-2- ethylbenzamide formate | 647.7 | 692.5 (M + HCOO)− | in analogy to the procedure for example 482: amide coupling of N-(3- aminopropyl)-2-ethyl-4-((3- (3-(trifluoromethyl)-1H- pyrazol-4-yl)imidazo[1,2- a]pyrazin-8- yl)amino)benzamide (example 482, step 2) and (1S,3R)-3-((tert- butoxycarbonyl)amino) cyclopentane-1-carboxylic acid [CAS#261165-05-3], alkylation with 1,1-difluoro- 2-iodoethane [CAS#598-39- 0] and subsequent acidic Boc deprotection |
| 486 | 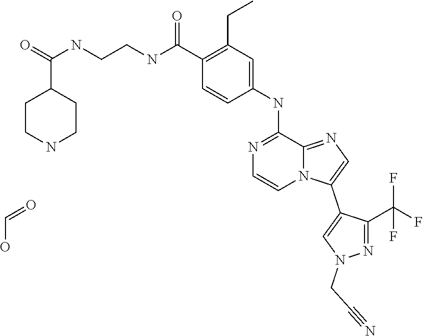 [SMILES: O=CO ; CCc1cc(Nc2nccn3c(-c4cn(CC#N)nc4C(F)(F)F)cnc23)ccc1C(=O)NCCNC(=O)C1CCNCC1] | N-(2-(4-((3-(1- (cyanomethyl)-3- (trifluoromethyl)- 1H-pyrazol-4- yl)imidazo[1,2- a]pyrazin-8- yl)amino)-2- ethylbenzamido) ethyl)piperidine- 4-carboxamide formate | 608.6 | 609.3 | in analogy to the procedure for example 481: amide coupling of N-(2- aminoethyl)-2-ethyl-4- [[3-[3- (trifluoromethyl)-1H- pyrazol- 4-yl]imidazo[1,2-a] pyrazin-8- yl]amino]benzamide (example 3, step 4) and1- (tert-butoxycarbonyl) piperidine-4- carboxylic acid [CAS#84358- 13-4], alkylation with 2- bromoacetonitrile [CAS#590- 17-0] and subsequent acidic Boc deprotection |
| 487 | 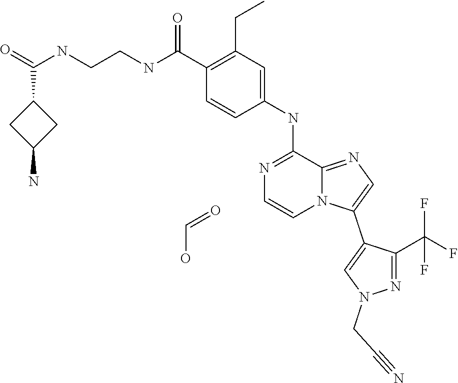 [SMILES: CCc1cc(Nc2nccn3c(-c4cn(CC#N)nc4C(F)(F)F)cnc23)ccc1C(=O)NCCNC(=O)[C@H]1C[C@H](N)C1 ; O=CO] | N-(2-((1r,3r)-3- aminocyclobutane- 1- carboxamido)ethyl)- 4-((3-(1- (cyanomethyl)-3- (trifluoromethyl)- 1H-pyrazol-4- yl)imidazo[1,2- a]pyrazin-8- yl)amino)-2- ethylbenzamide formate | 594.6 | 595.3 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1r,3r)-3-((tert- butoxycarbonyl)amino) cyclobutane-1-carboxylic acid [CAS#939400-34-7], alkylation with 2- bromoacetonitrile [CAS#590- 17-0] and subsequent acidic Boc deprotection |
| 488 | 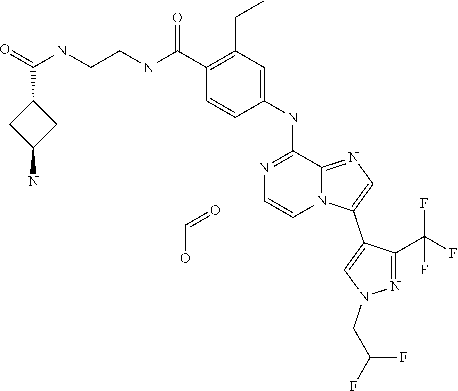 [SMILES: CCc1cc(Nc2nccn3c(-c4cn(CC(F)F)nc4C(F)(F)F)cnc23)ccc1C(=O)NCCNC(=O)[C@H]1C[C@H](N)C1 ; O=CO] | N-(2-((1r,3r)-3- aminocyclobutane- 1- carboxamido)ethyl)- 4-((3-(1-(2,2- difluoroethyl)-3- (trifluoromethyl)- 1H-pyrazol-4- yl)imidazo[1,2- a]pyrazin-8- yl)amino)-2- ethylbenzamide formate | 619.6 | 620.3 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1r,3r)-3-((tert- butoxycarbonyl)amino) cyclobutane-1-carboxylic acid [CAS#939400-34-7], alkylation with 1,1- difluoro- 2-iodoethane [CAS#598-39- 0] and subsequent acidic Boc deprotection |
| 489 | 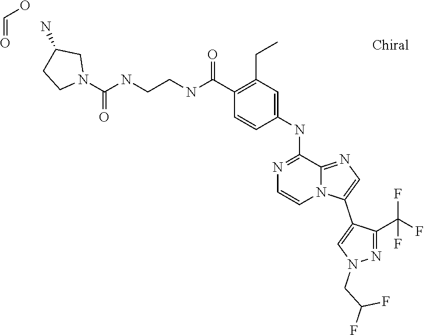 | (S)-3-amino-N-(2- (4-((3-(1-(2,2- difluoroethyl)-3- (trifluoromethyl)- 1H-pyrazol-4- yl)imidazo[1,2- a]pyrazin-8- yl)amino)-2- ethylbenzamido) ethyl)pyrrolidine-1- carboxamide formate | 634.6 | 635.4 | in analogy to the procedure for example11 step 3: urea formation of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and tert- butyl (S)-pyrrolidin-3- ylcarbamate [CAS#122536- 76-9], then in analogy to the procedure for example 481 alkylation with 1,1- difluoro-2-iodoethane [CAS#598-39- 0] and subsequent acidic Boc deprotection |
| 490 | 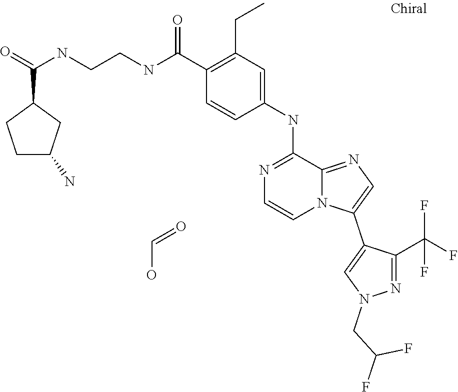 [SMILES: CCc1cc(Nc2nccn3c(-c4cn(CC(F)F)nc4C(F)(F)F)cnc23)ccc1C(=O)NCCNC(=O)[C@@H]1CC[C@@H](N)C1 ; O=CO] | N-(2-((1R,3R)-3- aminocyclopentane- 1- carboxamido)ethyl)- 4-((3-(1-(2,2- difluoroethyl)-3- (trifluoromethyl)- 1H-pyrazol-4- yl)imidazo[1,2- a]pyrazin-8- yl)amino)-2- ethylbenzamide formate | 633.6 | 632.5 (M − H)− | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1R,3R)-3-((tert- butoxycarbonyl)amino) cyclopentane-1-carboxylic acid [CAS#489446-85-7], alkylation with 1,1-difluoro-2-iodoethane [CAS#598-39- 0] and subsequent acidic Boc deprotection |
| 491 | 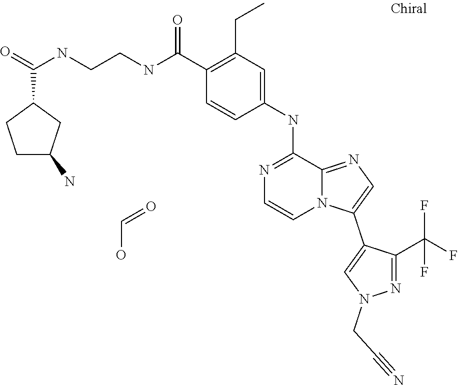 [SMILES: CCc1cc(Nc2nccn3c(-c4cn(CC#N)nc4C(F)(F)F)cnc23)ccc1C(=O)NCCNC(=O)[C@H]1CC[C@H](N)C1 ; O=CO] | N-(2-((1S,3S)-3- aminocyclopentane- 1- carboxamido)ethyl)- 4-((3-(1- (cyanomethyl)-3- (trifluoromethyl)- 1H-pyrazol-4- yl)imidazo[1,2- a]pyrazin-8- yl)amino)-2- ethylbenzamide formate | 608.6 | 607.4 (M − H)− | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl] amino]benzamide (example 3, step 4) and (1S,3S)-3-((tert- butoxycarbonyl)amino) cyclopentane-1-carboxylic acid [CAS#161601-29-2], alkylation with 2- bromoacetonitrile [CAS#590- 17-0] and subsequent acidic Boc deprotection |
| 492 | 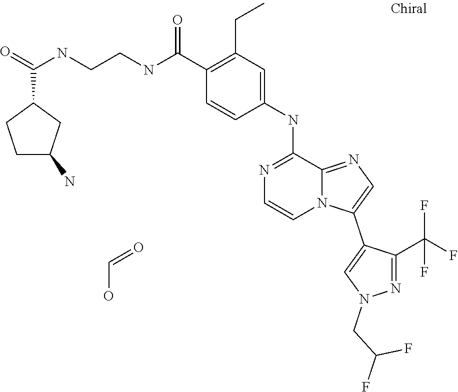 [SMILES: CCc1cc(Nc2nccn3c(-c4cn(CC(F)F)nc4C(F)(F)F)cnc23)ccc1C(=O)NCCNC(=O)[C@H]1CC[C@H](N)C1 ; O=CO] | N-(2-((1S,3S)-3- aminocyclopentane- 1- carboxamido)ethyl)- 4-((3-(1-(2,2- difluoroethyl)-3- (trifluoromethyl)- 1H-pyrazol-4- yl)imidazo[1,2- a]pyrazin-8- yl)amino)-2- ethylbenzamide formate | 633.6 | 632.4 (M − H)− | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1S,3S)-3-((tert- butoxycarbonyl)amino) cyclopentane-1-carboxylic acid [CAS#161601-29-2], alkylation with 1,1-difluoro- 2-iodoethane [CAS#598-39- 0] and subsequent acidic Boc deprotection |
| 493 | 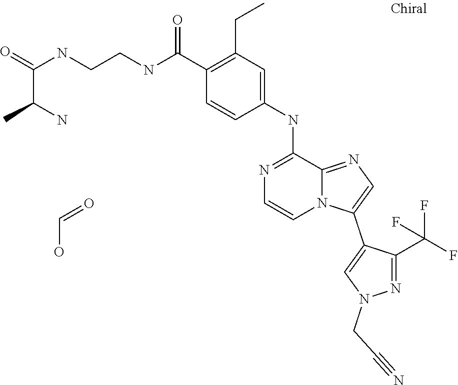 [SMILES: CCc1cc(Nc2nccn3c(-c4cn(CC#N)nc4C(F)(F)F)cnc23)ccc1C(=O)NCCNC(=O)[C@H](C)N ; O=CO] | (S)-N-(2-(2- aminopropanamido) ethyl)-4-((3-(1- (cyanomethyl)-3- (trifluoromethyl)- 1H-pyrazol-4- yl)imidazo[1,2- a]pyrazin-8- yl)amino)-2- ethylbenzamide formate | 568.6 | 569.3 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (tert- butoxycarbonyl)-L-alanine [CAS#15761-38-3], alkylation with 2- bromoacetonitrile [CAS#590- 17-0] and subsequent acidic Boc deprotection |
| 494 | 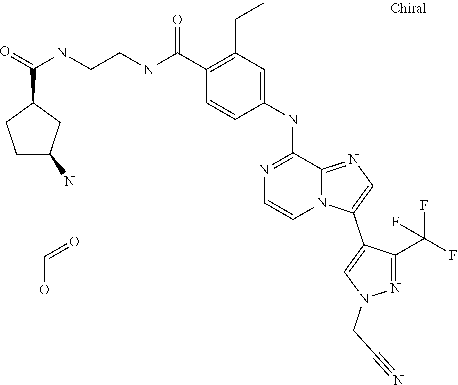 [SMILES: CCc1cc(Nc2nccn3c(-c4cn(CC#N)nc4C(F)(F)F)cnc23)ccc1C(=O)NCCNC(=O)[C@@H]1CC[C@H](N)C1 ; O=CO] | N-(2-((1R,3S)-3- aminocyclopentane- 1- carboxamido)ethyl)- 4-((3-(1- (cyanomethyl)-3- (trifluoromethyl)- 1H-pyrazol-4- yl)imidazo[1,2- a]pyrazin-8- yl)amino)-2- ethylbenzamide formate | 608.6 | 609.4 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1R,3S)-3-((tert- butoxycarbonyl)amino) cyclopentane-1-carboxylic acid [CAS #161660-94-2], alkylation with 2- bromoacetonitrile [CAS#590- 17-0] and subsequent acidic Boc deprotection |
| 495 | 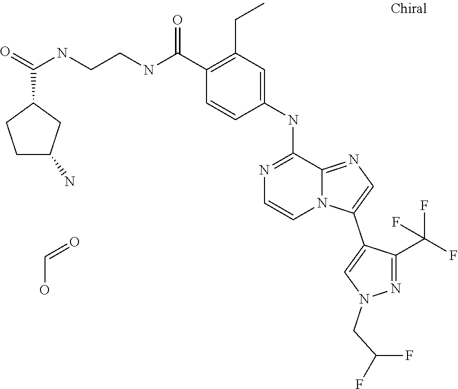 [SMILES: CCc1cc(Nc2nccn3c(-c4cn(CC(F)F)nc4C(F)(F)F)cnc23)ccc1C(=O)NCCNC(=O)[C@H]1CC[C@@H](N)C1 ; O=CO] | N-(2-((1S,3R)-3- aminocyclopentane- 1- carboxamido)ethyl)- 4-((3-(1-(2,2- difluoroethyl)-3- (trifluoromethyl)- 1H-pyrazol-4- yl)imidazo[1,2- a]pyrazin-8- yl)amino)-2- ethylbenzamide formate | 633.6 | 634.4 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1S,3R)-3-((tert- butoxycarbonyl)amino) cyclopentane-1-carboxylic acid [CAS #261165-05-3], alkylation with 1,1-difluoro- 2-iodoethane [CAS#598-39- 0] and subsequent acidic Boc deprotection |
| 496 | 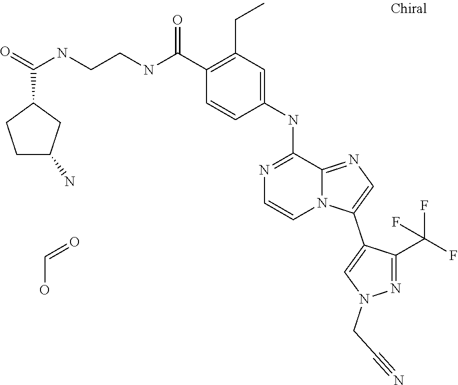 [SMILES: CCc1cc(Nc2nccn3c(-c4cn(CC#N)nc4C(F)(F)F)cnc23)ccc1C(=O)NCCNC(=O)[C@H]1CC[C@@H](N)C1 ; O=CO] | N-(2-((1S,3R)-3- aminocyclopentane- 1- carboxamido)ethyl)- 4-((3-(1- (cyanomethyl)-3- (trifluoromethyl)- 1H-pyrazol-4- yl)imidazo[1,2- a]pyrazin-8- yl)amino)-2- ethylbenzamide formate | 608.6 | 609.4 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1S,3R)-3-((tert- butoxycarbonyl)amino) cyclopentane-1-carboxylic acid [CAS #261165-05-3], alkylation with 2- bromoacetonitrile [ CAS#590- 17-0] and subsequent acidic Boc deprotection |
| 497 | 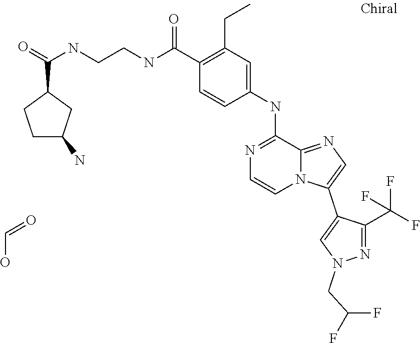 [SMILES: O=CO ; CCc1cc(Nc2nccn3c(-c4cn(CC(F)F)nc4C(F)(F)F)cnc23)ccc1C(=O)NCCNC(=O)[C@@H]1CC[C@H](N)C1] | N-(2-((1R,3S)-3- aminocyclopentane- 1- carboxamido)ethyl)- 4-((3-(1-(2,2- difluoroethyl)-3- (trifluoromethyl)- 1H-pyrazol-4- yl)imidazo[1,2- a]pyrazin-8- yl)amino)-2- ethylbenzamide formate | 633.6 | 634.4 | in analogy to the procedure for example 481: amide coupling of N-(2- aminoethyl)-2-ethyl- 4-[[3-[3- (trifluoromethyl)- 1H-pyrazol- 4-yl]imidazo[1,2-a] pyrazin-8- yl]amino]benzamide (example 3, step 4) and (1R,3S)-3-((tert- butoxycarbonyl)amino) cyclopentane-1-carboxylic acid [CAS #161660-94-2], alkylation with 1,1-difluoro- 2-iodoethane [CAS#598-39- 0] and subsequent acidic Boc deprotection |
| 498 | 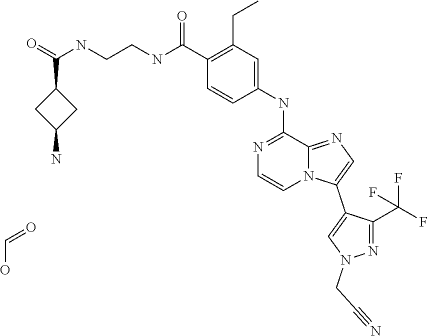 [SMILES: O=CO ; CCc1cc(Nc2nccn3c(-c4cn(CC#N)nc4C(F)(F)F)cnc23)ccc1C(=O)NCCNC(=O)[C@H]1C[C@@H](N)C1] | cis-N-(2-((1s,3s)-3- aminocyclobutane- 1- carboxamido)ethyl)- 4-((3-(1- (cyanomethyl)-3- (trifluoromethyl)- 1H-pyrazol-4- yl)imidazo[1,2- a]pyrazin-8- yl)amino)-2- ethylbenzamide formate | 594.6 | 595.3 | in analogy to the procedure for example 481: amide coupling of N-(2- aminoethyl)-2- ethyl-4-[[3-[3- (trifluoromethyl)- 1H-pyrazol- 4-yl]imidazo[1,2-a] pyrazin-8- yl]amino]benzamide (example 3, step 4) and (ls,3s)-3-((tert- butoxycarbonyl)amino) cyclobutane-1-carboxylic acid [1008773-79-2], alkylation with 2-bromoacetonitrile [CAS#590-17-0] and subsequent acidic Boc deprotection |

### Example 499

### 4-[[3-[1-(cyanomethyl)-3-(trifluoromethyl)pyrazol-4-yl]imidazo[1,2-a]pyrazin-8-yl]amino]-2-ethyl-N-(2-oxo-2-piperazin-1-yl-ethyl)benzamide formate

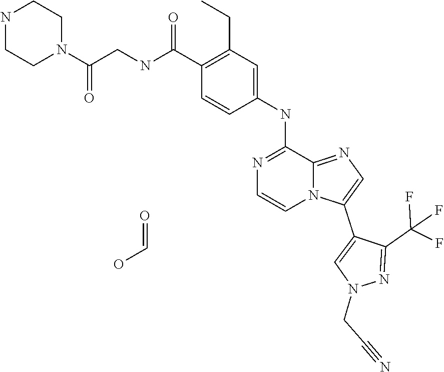 [SMILES: CCc1cc(Nc2nccn3c(-c4cn(CC#N)nc4C(F)(F)F)cnc23)ccc1C(=O)NCC(=O)N1CCNCC1 ; O=CO]

Step 1:

### 2-[[2-ethyl-4-[(3-iodoimidazo[1,2-a]pyrazin-8-yl)amino]benzoyl]amino]acetate

To a solution of 2-ethyl-4-[(3-iodoimidazo[1,2-a]pyrazin-8-yl)amino]benzoic acid (example 1, step 2) (3 g, 7.35 mmol), glycine ethyl ester hydrochloride (2.05 g, 14.7 mmol) and N,N-diisopropylethylamine (5.12 mL, 29.4 mmol) in DMF (30 mL) was added 0-(7-azabenzotriazol-1-yl)-N,N,N′,N′-tetramethyluronium hexafluorophosphate (4.19 g, 11.02 mmol). The reaction was stirred at 20° C. for 16 h. The reaction mixture was diluted with water (50 mL) and extracted with EtOAc (50 mL×3). The combined organic layer was washed with brine (100 mL), dried over Na₂SO₄, filtered and concentrated to yield the title compound as a yellow solid (4 g, 8.11 mmol, 110.33% yield). MS(m/e): 494.2 (M+H).

Step 2:

### 2-[[2-ethyl-4-[(3-iodoimidazo[1,2-a]pyrazin-8-yl)amino]benzoyl]amino]acetic acid

To a solution of ethyl 2-[[2-ethyl-4-[(3-iodoimidazo[1,2-a]pyrazin-8-yl)amino]benzoyl]amino]acetate (4 g, 8.11 mmol) in THF (30 mL)/methanol (30 mL) was added aqueous lithium hydroxide (30 mL, 30 mmol). The reaction was stirred at 20° C. for 0.33 h. The reaction mixture was adjusted to pH=3 with 3N HCl solution. A lot of solid was formed, so it was filtered and collected. The filter cake was dried to yield the title compound as an off-white solid (3.7 g, 7.95 mmol, 98.08% yield). MS(m/e): 466.2 (M+H).

Step 3:

### 4-iodo-3-(trifluoromethyl)-1H-pyrazole

To a solution of 3-(trifluoromethyl)pyrazole (74 g, 543.8 mmol) in aqueous sulfuric acid (160 mL, 543.8 mmol) was added N-iodosuccinimide (146.81 g, 652.56 mmol) at 0° C. and it was stirred for 10 min. Then it was warmed to 20° C. and stirred for 1 h. Water (2 L) was added to the mixture and it was stirred at 20° C. for 12 h. The reaction mixture was filtered. The filter cake was dissolved in EtOAc (1.5 L) and washed with saturated Na₂SO₃ (1 L). The organic layer was washed with brine (1 L), dried over Na₂SO₄, filtered and concentrated. The residue was purified by column chromatography (SiO₂, PE:EtOAc=3:1) to yield the title compound as a white solid (125.6 g, 479.44 mmol, 88.17% yield). MS(m/e): 262.9 (M+H).

Step 4:

### 2-[4-iodo-3-(trifluoromethyl)pyrazol-1-yl]acetonitrile

To a mixture of 4-iodo-3-(trifluoromethyl)-1H-pyrazole (30 g, 114.52 mmol) in acetone (600 mL) was added potassium carbonate (18.99 g, 137.42 mmol) and 2-bromoacetonitrile (16.48 g, 137.42 mmol) at −60° C. The mixture was stirred at 20° C. for 4 h. The mixture was poured into water (200 mL). The aqueous phase was extracted with EtOAc (200 mL×2). The combined organic phase was washed with brine (300 mL), dried over anhydrous Na₂SO₄ and concentrated in vacuum. The residue was purified by silica gel column chromatography (petroleum ether/EtOAc=20/1) to yield the title compound as a colorless oil (30 g, 99.66 mmol, 87.03% yield). MS(m/e): 334.9 (M+H).

Step 5:

### 2-[4-(4,4,5,5-tetramethyl-1,3,2-dioxaborolan-2-yl)-3-(trifluoromethyl)pyrazol-1-yl]acetonitrile

To a solution of 2-[4-iodo-3-(trifluoromethyl)pyrazol-1-yl]acetonitrile (10 g, 33.22 mmol) in DMF (100 mL) was added 1,1′-bis(diphenylphosphino)ferrocene-palladium(II)dichloride dichloromethane complex (4.07 g, 4.98 mmol), potassium acetate (9.78 g, 99.66 mmol) and bis(pinacolato)diboron (12.65 g, 49.83 mmol) under N₂, then the reaction was stirred at 80° C. for 16 h. The reaction mixture was poured into water (400 ml) and extracted with EtOAc (200 mL×3). The combined organic phase was washed with brine (200 mL×3) and dried over anhydrous Na₂SO₄, filtered and concentrated. The residue was purified by silica gel column chromatography eluted with PE:EtOAc=20:1 to 5:1 to yield the title compound as a yellow oil (8.6 g, 28.56 mmol, 85.98% yield, 60% purity).

Step 6:

### 2-[[4-[[3-[1-(cyanomethyl)-3-(trifluoromethyl)pyrazol-4-yl]imidazo[1,2-a]pyrazin-8-yl]amino]-2-ethyl-benzoyl]amino]acetic acid

To a solution of 2-[4-(4,4,5,5-tetramethyl-1,3,2-dioxaborolan-2-yl)-3-(trifluoromethyl)pyrazol-1-yl]acetonitrile (4.31 g, 8.6 mmol) and 2-[[2-ethyl-4-[(3-iodoimidazo[1,2-a]pyrazin-8-yl)amino]benzoyl]amino]acetic acid (2 g, 4.3 mmol) in DMSO (20 mL) was added 1,1′-bis(diphenylphosphino)ferrocene-palladium(ii)dichloride dichloromethane complex (350.78 mg, 0.430 mmol) and sodium carbonate (1 g, 9.46 mmol). The reaction was stirred at 80° C. under N₂ for 12 h. The reaction mixture was diluted with water (50 mL) and adjusted to pH=4 with formic acid. Then EtOAc (100 mL) was added and the mixture was stirred and filtered. The filtrate was extracted with EtOAc (50 mL). The organic layer was washed with brine (100 mL), dried over Na₂SO₄, filtered and concentrated. The residue was diluted with water (50 mL) and adjusted to pH=10 with DIPEA. Then it was extracted with EtOAc (100 mL). The aqueous layer was adjusted to pH=4 with formic acid and filtered. The filter cake was dried to yield the title compound as a brown solid (900 mg, 1.76 mmol, 40.86% yield). MS(m/e): 513.2 (M+H).

Step 7:

### tert-butyl 4-[2-[[4-[[3-[1-(cyanomethyl)-3-(trifluoromethyl)pyrazol-4-yl]imidazo[1,2-a]pyrazin-8-yl]amino]-2-ethyl-benzoyl]amino]acetyl]piperazine-1-carboxylate

To a solution of 2-[[4-[[3-[1-(cyanomethyl)-3-(trifluoromethyl)pyrazol-4-yl]imidazo[1,2-a]pyrazin-8-yl]amino]-2-ethyl-benzoyl]amino]acetic acid (90 mg, 0.180 mmol), 1-Boc-piperazine (65.42 mg, 0.350 mmol), N,N-diisopropylethylamine (0.09 mL, 0.530 mmol) in DMF (1 mL) was added 0-(7-azabenzotriazol-1-yl)-N,N,N′,N′-tetramethyluronium hexafluorophosphate (100.17 mg, 0.260 mmol). The reaction was stirred at 20° C. for 12 h. The reaction mixture was diluted with water (20 mL) and extracted with EtOAc (20 mL×3). The combined organic layer was washed with brine (50 mL), dried over Na₂SO₄, filtered and concentrated. The residue was purified by Prep-TLC (DCM:MeOH=10:1) to yield the title compound as a yellow oil (50 mg, 0.070 mmol, 41.82% yield). MS(m/e): 681.4 (M+H).

Step 8:

### 4-[[3-[1-(cyanomethyl)-3-(trifluoromethyl)pyrazol-4-yl]imidazo[1,2-a]pyrazin-8-yl]amino]-2-ethyl-N-(2-oxo-2-piperazin-1-yl-ethyl)benzamide formate

To a solution of tert-butyl 4-[2-[[4-[[3-[1-(cyanomethyl)-3-(trifluoromethyl)pyrazol-4-yl]imidazo[1,2-a]pyrazin-8-yl]amino]-2-ethyl-benzoyl]amino]acetyl]piperazine-1-carboxylate (50 mg, 0.070 mmol) in DCM (5 mL) was added trifluoroacetic acid (0.5 mL, 6.49 mmol). The reaction was stirred at 20° C. for 1 h. The reaction mixture was concentrated and adjusted to pH=7 with Et₃N. The residue was purified by Prep-HPLC (with formic acid) to yield the title compound as a white solid (17 mg, 0.030 mmol, 35.13% yield). MS(mi/e): 581.2 (M+H).

The following examples were synthesized in analogy to procedures described before. The reaction sequence and staring materials are recorded in the Table 3.

TABLE 3

| | | | | MW | |
|---|---|---|---|---|---|
| | | Systematic | Parent | (M + H) | Reaction Sequence and |
| No | Stucture | Name | MW | found | Starting Materials |
| 500 | 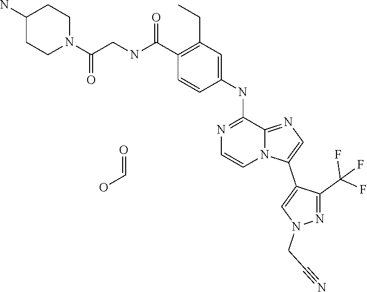 [SMILES: CCc1cc(Nc2nccn3c(-c4cn(CC#N)nc4C(F)(F)F)cnc23)ccc1C(=O)NCC(=O)N1CCC(N)CC1 ; O=CO] | N-[2-(4-amino-1- piperidyl)-2-oxo- ethyl]-4-[[3-[1- (cyanomethyl)-3- (trifluoromethyl) pyrazol-4- yl]imidazo[1,2- a]pyrazin-8- yl]amino]-2-ethyl- benzamide formate | 594.6 | 595.1 | in analogy to the procedure for example 499: amide coupling of 2-[[4-[[3-[1- (cyanomethyl)-3- (trifluoromethyl)pyrazol-4- yl]imidazo[1,2-a]pyrazin-8- yl]amino]-2-ethyl- benzoyl]amino]acetic acid (example 499, step 6) with 4- Boc-aminopiperidine [CAS#73874-95-0] and subsequent acidic Boc deprotection |
| 501 | 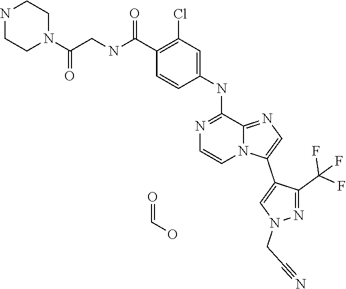 [SMILES: N#CCn1cc(-c2cnc3c(Nc4ccc(C(=O)NCC(=O)N5CCNCC5)c(Cl)c4)nccn23)c(C(F)(F)F)n1 ; O=CO] | 2-chloro-4-[[3-[1- (cyanomethyl)-3- (trifluoromethyl) pyrazol-4- yl]imidazo[1,2- a]pyrazin-8- yl]amino]-N-(2-oxo- 2-piperazin-1-yl- ethyl)benzamide formate | 587.0 | 587.1 | in analogy to the procedure for example 499: saponification of methyl 2- chloro-4-[(3-iodoimidazo[1,2- a]pyrazin-8- yl)amino]benzoate (example 6, step 1) with NaOH, then amide coupling with glycine ethyl ester hydrochloride, hydrolysis and amide coupling with 1-Boc- piperazine [CAS#57260-71- 6], Suzuki reaction with 2-[4- (4,4,5,5-tetramethyl-1,3,2- dioxaborolan-2-yl)-3- (trifluoro methyl)pyrazol-1- yl]acetonitrile (example 499, step 5) and acidic Boc deprotection |
| 502 | 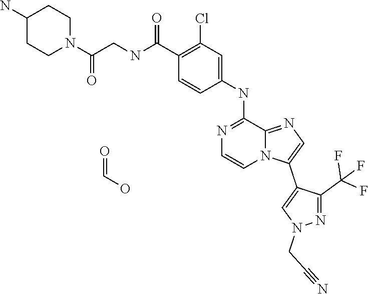 [SMILES: N#CCn1cc(-c2cnc3c(Nc4ccc(C(=O)NCC(=O)N5CCC(N)CC5)c(Cl)c4)nccn23)c(C(F)(F)F)n1 ; O=CO] | N-[2-(4-amino-1- piperidyl)-2-oxo- ethyl]-2-chloro-4- [[3-[1- (cyanomethyl)-3- (trifluoromethyl) pyrazol-4- yl]imidazo[1,2- a]pyrazin-8- yl]amino]benzamide formate | 601.0 | 601.1 | in analogy to the procedure for example 499: saponification of methyl 2- chloro-4-[(3-iodoimidazo[1,2- a]pyrazin-8- yl)amino]benzoate (example 6, step 1) with NaOH, then amide coupling with glycine ethyl ester hydrochloride, hydrolysis and amide coupling with 4-Boc- aminopiperidine [CAS#73874-95-0], Suzuki reaction with 2-[4-(4,4,5,5- tetramethyl-1,3,2- dioxaborolan-2-yl)-3- (trifluoro methyl)pyrazol-1- yl]acetonitrile (example 499, step 5) and acidic Boc deprotection |
| 503 | 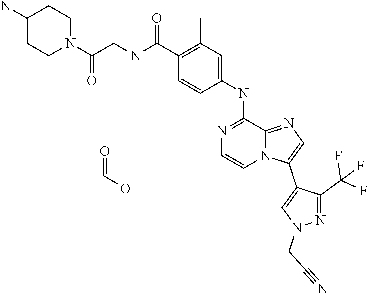 [SMILES: Cc1cc(Nc2nccn3c(-c4cn(CC#N)nc4C(F)(F)F)cnc23)ccc1C(=O)NCC(=O)N1CCC(N)CC1 ; O=CO] | N-[2-(4-amino-1- piperidyl)-2-oxo- ethyl]-4-[[3-[1- (cyanomethyl)-3- (trifluoromethyl) pyrazol-4- yl]imidazo[1,2- a]pyrazin-8- yl]amino]-2- methyl-benzamide formate | 580.6 | 581.3 | in analogy to the procedure for example 499: amide coupling of 4-[(3- iodoimidazo[1,2-a]pyrazin-8- yl)amino]-2-methyl-benzoic acid (example 16, step 1) with glycine ethyl ester hydrochloride, saponification with LiOH, Suzuki reaction with 2-[4-(4,4,5,5- tetramethyl-1,3,2- dioxaborolan-2-yl)-3- (trifluoro methyl)pyrazol-1- yl]acetonitrile (example 499, step 5), amide coupling with 4-Boc-aminopiperidine [CAS#73874-95-0] and subsequent acidic Boc deprotection |
| 504 | 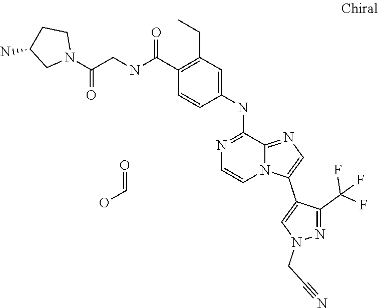 [SMILES: CCc1cc(Nc2nccn3c(-c4cn(CC#N)nc4C(F)(F)F)cnc23)ccc1C(=O)NCC(=O)N1CC[C@@H](N)C1 ; O=CO] | N-[2-[(3R)-3- aminopyrrolidin- 1-yl]-2-oxo- ethyl]-4-[[3-[1- (cyanomethyl)-3- (trifluoromethyl)p yrazol-4- yl]imidazo[1,2- a]pyrazin-8- yl]amino]-2-ethyl- benzamide formate | 580.6 | 581.2 | in analogy to the procedure for example 499: amide coupling of 2-[[4-[[3-[1- (cyanomethyl)-3- (trifluoromethyl)pyrazol-4- yl]imidazo[1,2-a]pyrazin-8- yl]amino]-2-ethyl- benzoyl] amino] acetic acid (example 499, step 6) with (R)-3-(Boc-amino)pyrrolidine [CAS#122536-77-0] and subsequent acidic Boc deprotection |

The following compounds of Table 4 were prepared in analogy to structurally similar compounds described above.

TABLE 4

| | | | | MW |
|---|---|---|---|---|
| | | | | (M + |
| | | | Parent | H) |
| No | Structure | Systematic Name | MW | found |
| 505 | 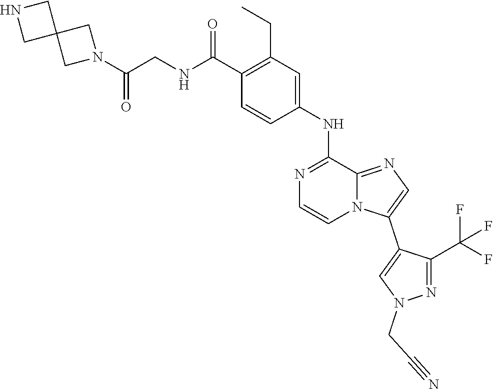 [SMILES: CCc1cc(Nc2nccn3c(-c4cn(CC#N)nc4C(F)(F)F)cnc23)ccc1C(=O)NCC(=O)N1CC2(CNC2)C1] | 4-[[3-[1-(cyanomethyl)-3- (trifluoromethyl)pyrazol-4- yl]imidazo[1,2-a]pyrazin-8- yl]amino]-N-[2-(2,6- diazaspiro[3.3]heptan- 2-yl)-2-oxo- ethyl]-2-ethyl-benzamide formate | 592.6 | 593.3 |
| 506 | 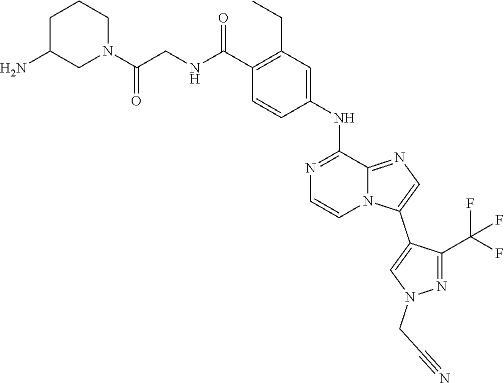 [SMILES: CCc1cc(Nc2nccn3c(-c4cn(CC#N)nc4C(F)(F)F)cnc23)ccc1C(=O)NCC(=O)N1CCCC(N)C1] | N-[2-(3-amino-1- piperidyl)- 2-oxo- ethyl]-4-[[3-[1- (cyanomethyl)-3- (trifluoromethyl)pyrazol-4- yl]imidazo[1,2-a]pyrazin-8- yl]amino]-2-ethyl- benzamide formate | 594.6 | 595.3 |
| 507 | 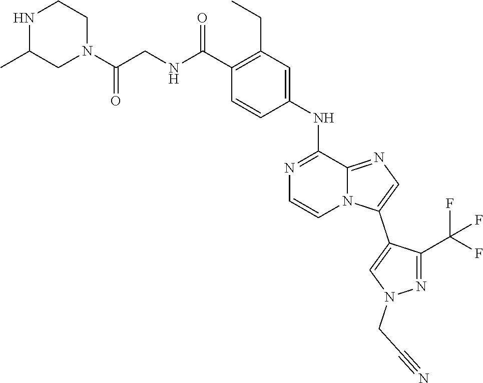 [SMILES: CCc1cc(Nc2nccn3c(-c4cn(CC#N)nc4C(F)(F)F)cnc23)ccc1C(=O)NCC(=O)N1CCNC(C)C1] | 4-[[3-[1-(cyanomethyl)-3- (trifluoromethyl)pyrazol-4- yl]imidazo[1,2-a]pyrazin-8- yl]amino]-2-ethyl-N-[2-(3- methylpiperazin-1- yl)-2-oxo- ethyl]benzamide formate | 594.6 | 595.3 |
| 508 | 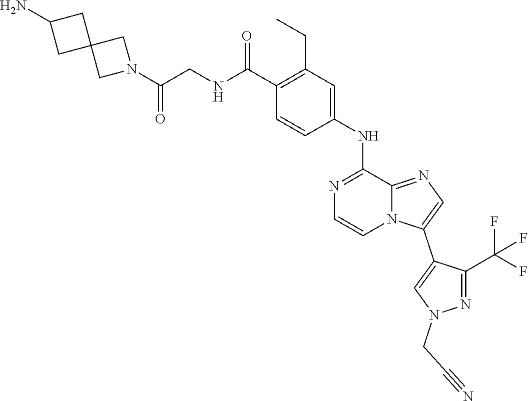 [SMILES: CCc1cc(Nc2nccn3c(-c4cn(CC#N)nc4C(F)(F)F)cnc23)ccc1C(=O)NCC(=O)N1CC2(CC(N)C2)C1] | N-[2-(6-amino-2- azaspiro[3.3]heptan- 2-yl)-2-oxo- ethyl]-4-[[3-[1- (cyanomethyl)-3- (trifluoromethyl) pyrazol-4- yl]imidazo[1,2-a] pyrazin-8- yl]amino]-2-ethyl- benzamide formate | 606.6 | 607.3 |
| 509 | 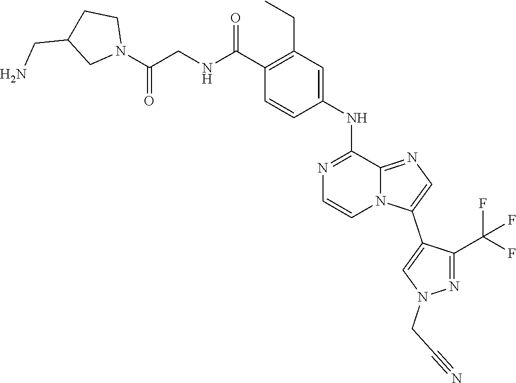 [SMILES: CCc1cc(Nc2nccn3c(-c4cn(CC#N)nc4C(F)(F)F)cnc23)ccc1C(=O)NCC(=O)N1CCC(CN)C1] | N-[2-[3-(aminomethyl) pyrrolidin-1- yl]-2-oxo-ethyl]-4-[[3-[1- (cyanomethyl)-3- (trifluoromethyl) pyrazol-4- yl]imidazo[1,2-a]pyrazin-8- yl]amino]-2-ethyl- benzamide formate | 594.6 | 595.3 |
| 510 | 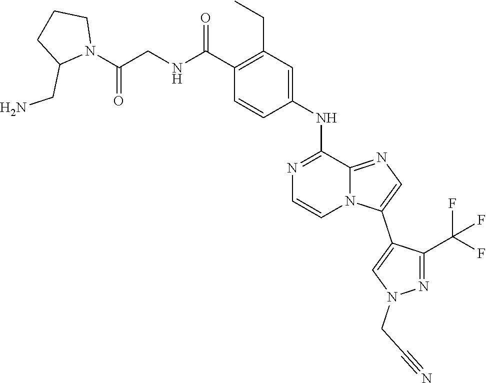 [SMILES: CCc1cc(Nc2nccn3c(-c4cn(CC#N)nc4C(F)(F)F)cnc23)ccc1C(=O)NCC(=O)N1CCCC1CN] | N-[2-[2-(aminomethyl) pyrrolidin-1- yl]-2-oxo-ethyl]-4-[[3-[1- (cyanomethyl)-3- (trifluoromethyl)pyrazol-4- yl]imidazo[1,2-a]pyrazin-8- yl]amino]-2-ethyl- benzamide formate | 594.6 | 595.2 |
| 511 | 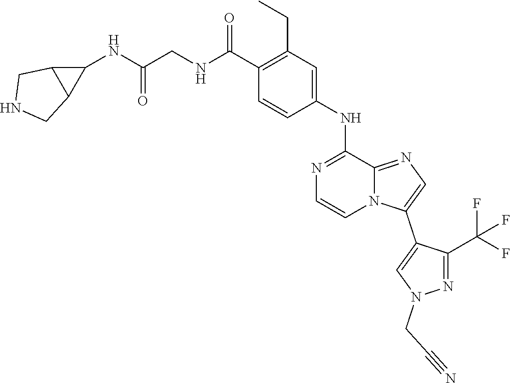 [SMILES: CCc1cc(Nc2nccn3c(-c4cn(CC#N)nc4C(F)(F)F)cnc23)ccc1C(=O)NCC(=O)NC1C2CNCC21] | N-[2-(3-azabicyclo[3.1.0] hexan-6- ylamino)-2-oxo- ethyl]-4-[[3-[1- (cyanomethyl)-3- (trifluoromethyl)pyrazol-4- yl]imidazo[1,2-a] pyrazin-8- yl]amino]-2-ethyl- benzamide formate | 592.6 | 593.2 |
| 512 | 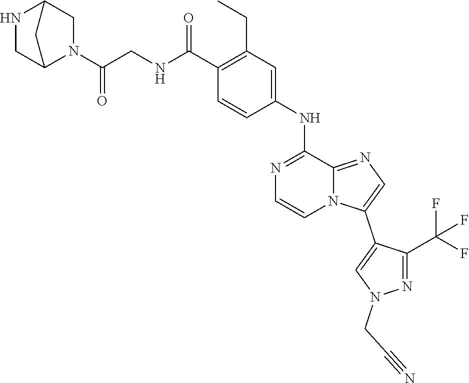 [SMILES: CCc1cc(Nc2nccn3c(-c4cn(CC#N)nc4C(F)(F)F)cnc23)ccc1C(=O)NCC(=O)N1CC2CC1CN2] | 4-[[3-[1-(cyanomethyl)-3- (trifluoromethyl)pyrazol-4- yl]imidazo[1,2-a]pyrazin-8- yl]amino]-N-[2-(2,5- diazabicyclo[2.2.1]heptan- 2-yl)-2- oxo-ethyl]-2-ethyl- benzamide formate | 592.6 | 593.2 |
| 513 | 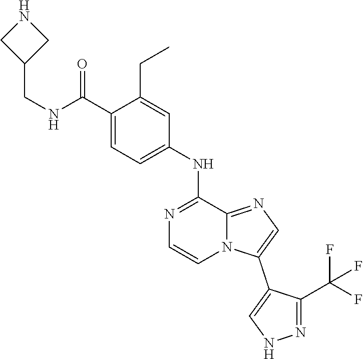 [SMILES: CCc1cc(Nc2nccn3c(-c4c[nH]nc4C(F)(F)F)cnc23)ccc1C(=O)NCC1CNC1] | N-(azetidin-3-ylmethyl)- 2-ethyl-4- ((3-(3-(trifluoromethyl)-1H- pyrazol-4-yl)imidazo[1,2-a] pyrazin- 8-yl)amino)benzamide formate | 484.5 | 485.3 |
| 514 | 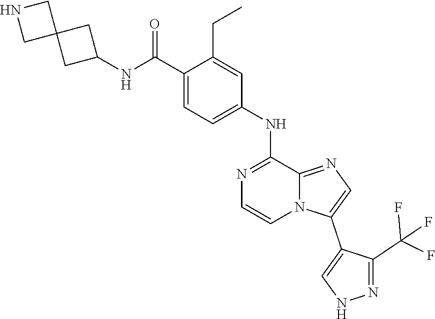 [SMILES: CCc1cc(Nc2nccn3c(-c4c[nH]nc4C(F)(F)F)cnc23)ccc1C(=O)NC1CC2(CNC2)C1] | 2-ethyl-N-(2-azaspiro[3.3] heptan-6- yl)-4-((3-(3- (trifluoromethyl)-1H- pyrazol-4-yl)imidazo[1,2-a] pyrazin- 8-yl)amino)benzamide formate | 510.5 | 511.3 |
| 515 | 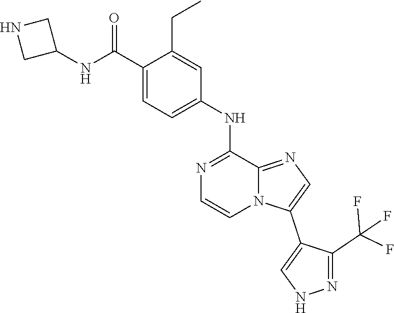 [SMILES: CCc1cc(Nc2nccn3c(-c4c[nH]nc4C(F)(F)F)cnc23)ccc1C(=O)NC1CNC1] | N-(azetidin-3-yl)-2-ethyl- 4-((3-(3- (trifluoromethyl)-1H- pyrazol-4- yl)imidazo[1,2-a]pyrazin-8- yl)amino)benzamide formate | 470.5 | 471.2 |
| 516 | 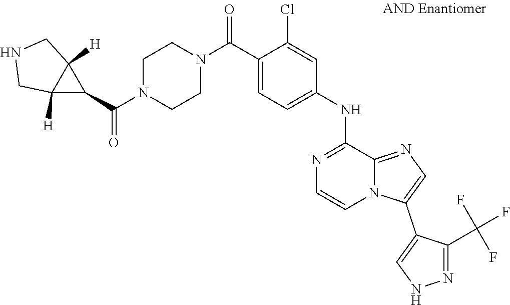 [SMILES: O=C(c1ccc(Nc2nccn3c(-c4c[nH]nc4C(F)(F)F)cnc23)cc1Cl)N1CCN(C(=O)[C@H]2[C@@H]3CNC[C@@H]32)CC1] | (4-((1R,5S,6r)-3- azabicyclo[3.1.0] hexane-6- earbonyl)piperazin-1-yl) (2-chloro- 4-((3-(3- (trifluoromethyl)-1H- pyrazol-4-yl) imidazo[1,2-a] pyrazin- 8-yl)amino) phenyl)methanone formate | 600.0 | 598.4 [M − H]− ESI neg |
| 517 | 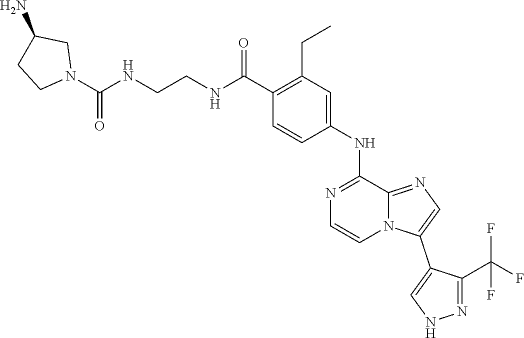 [SMILES: CCc1cc(Nc2nccn3c(-c4c[nH]nc4C(F)(F)F)cnc23)ccc1C(=O)NCCNC(=O)N1CC[C@@H](N)C1] | (R)-3-amino-N-(2-(2-ethyl- 4-((3-(3- (trifluoromethyl)- 1H-pyrazol-4- yl)imidazo[1,2-a]pyrazin-8- yl)amino)benzamido)ethyl) pyrrolidine-1-carboxamide formate | 570.6 | 571.3 |
| 519 | 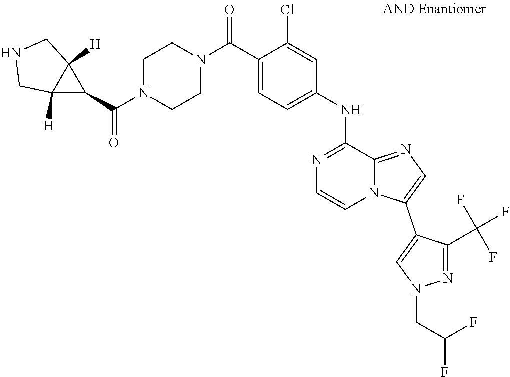 [SMILES: O=C(c1ccc(Nc2nccn3c(-c4cn(CC(F)F)nc4C(F)(F)F)cnc23)cc1Cl)N1CCN(C(=O)[C@H]2[C@@H]3CNC[C@@H]32)CC1] | (4-((1R,5S,6r)-3- azabicyclo[3.1.0]hexane- 6-carbonyl)piperazin- 1-yl)(2-chloro- 4-((3-(1-(2,2- difluoroethyl)-3- (trifluoromethyl)- 1H-pyrazol-4- yl)imidazo[1,2-a] pyrazin-8- yl)amino)phenyl) methanone formate | 664.0 | 708.4 [M + HCOO]−, ESI neg |
| 520 | 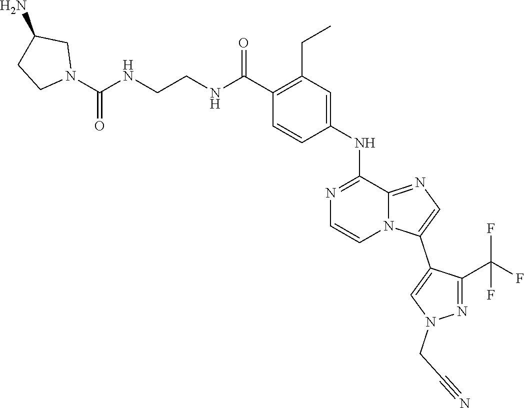 [SMILES: CCc1cc(Nc2nccn3c(-c4cn(CC#N)nc4C(F)(F)F)cnc23)ccc1C(=O)NCCNC(=O)N1CC[C@@H](N)C1] | (R)-3-amino-N-(2-(4- ((3-(1- (cyanomethyl)-3- (trifluoromethyl)- 1H-pyrazol-4-yl)imidazo [1,2-a]pyrazin-8-yl)amino)- 2-ethylbenzamido)ethyl) pyrrolidine- 1-carboxamide formate | 609.6 | 610.4 |
| 521 | 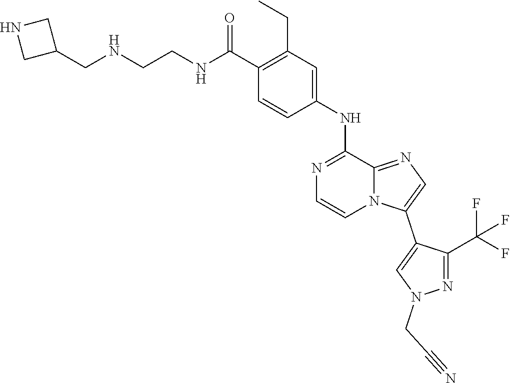 [SMILES: CCc1cc(Nc2nccn3c(-c4cn(CC#N)nc4C(F)(F)F)cnc23)ccc1C(=O)NCCNCC1CNC1] | N-[2-(azetidin-3- ylmethylamino)-2- oxo-ethyl]-4-[[3-[1- (cyanomethyl)- 3-(trifluoromethyl) pyrazol-4- yl]imidazo[1,2-a]pyrazin- 8-yl]amino]-2-ethyl- benzamide formate | 580.6 | 581.3 |
| 522 | 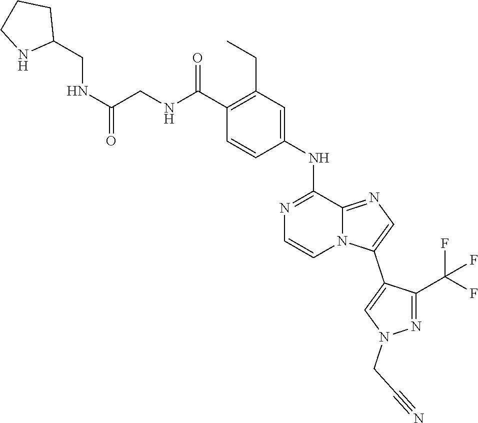 [SMILES: CCc1cc(Nc2nccn3c(-c4cn(CC#N)nc4C(F)(F)F)cnc23)ccc1C(=O)NCC(=O)NCC1CCCN1] | 4-[[3-[1-(cyanomethyl)-3- (trifluoromethyl)pyrazol-4- yl]imidazo[1,2-a] pyrazin-8- yl]amino]-2-ethyl- N-[2-oxo-2- (pyrrolidin-2- ylmethylamino)ethyl] benzamide formate | 594.6 | 595.2 |
| 523 | 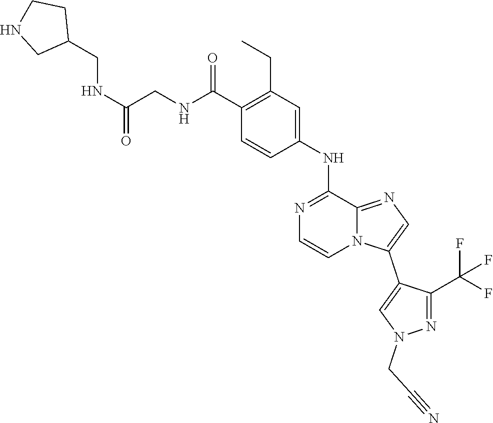 [SMILES: CCc1cc(Nc2nccn3c(-c4cn(CC#N)nc4C(F)(F)F)cnc23)ccc1C(=O)NCC(=O)NCC1CCNC1] | 4-[[3-[1-(cyanomethyl)-3- (trifluoromethyl)pyrazol-4- yl]imidazo[1,2-a]pyrazin-8- yl]amino]-2-ethyl-N- [2-oxo-2- (pyrrolidin-3- ylmethylamino)ethyl] benzamide formate | 594.6 | 595.2 |
| 524 | 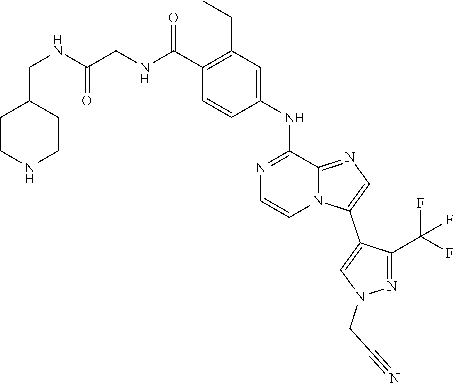 [SMILES: CCc1cc(Nc2nccn3c(-c4cn(CC#N)nc4C(F)(F)F)cnc23)ccc1C(=O)NCC(=O)NCC1CCNCC1] | 4-[[3-[1-(cyanomethyl)-3- (trifluoromethyl)pyrazol-4- yl]imidazo[1,2-a]pyrazin-8- yl]amino]-2-ethyl-N- [2-oxo-2-(4- piperidylmethylamino) ethyl] benzamide formate | 608.6 | 609.3 |
| 525 | 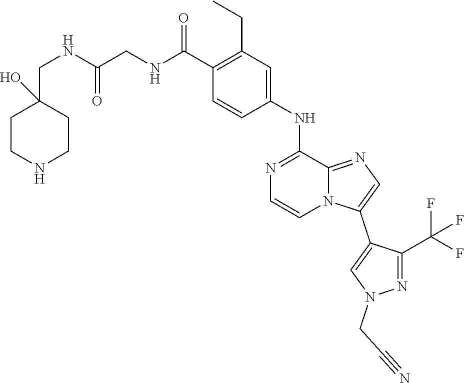 [SMILES: CCc1cc(Nc2nccn3c(-c4cn(CC#N)nc4C(F)(F)F)cnc23)ccc1C(=O)NCC(=O)NCC1(O)CCNCC1] | 4-[[3-[1-(cyanomethyl)-3- (trifluoromethyl)pyrazol-4- yl]imidazo[1,2-a]pyrazin-8- yl]amino]-2-ethyl-N-[2-[(4- hydroxy-4-piperidyl) methylamino]- 2-oxo-ethyl]benzamide formate | 624.6 | 625.3 |
| 526 | 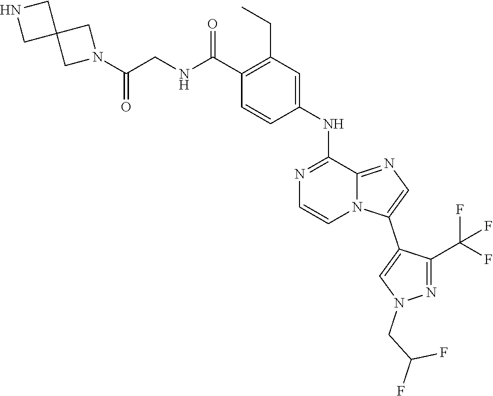 [SMILES: CCc1cc(Nc2nccn3c(-c4cn(CC(F)F)nc4C(F)(F)F)cnc23)ccc1C(=O)NCC(=O)N1CC2(CNC2)C1] | N-[2-(2,6-diazaspiro[3.3] heptan-2- yl)-2-oxo-ethyl]-4- [[3-[1-(2,2- difluoroethyl)-3- (trifluoromethyl)pyrazol-4- yl]imidazo[1,2-a]pyrazin-8- yl]amino]-2-ethyl- benzamide formate | 617.6 | 618.3 |
| 527 | 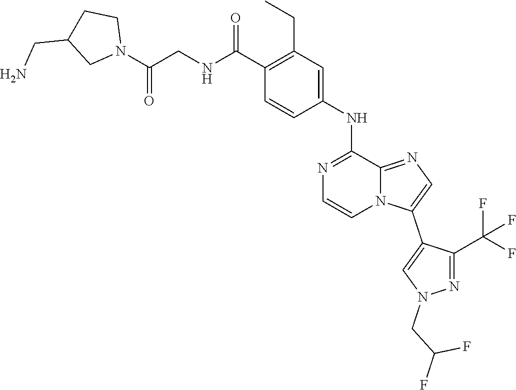 [SMILES: CCc1cc(Nc2nccn3c(-c4cn(CC(F)F)nc4C(F)(F)F)cnc23)ccc1C(=O)NCC(=O)N1CCC(CN)C1] | N-[2-[3-(aminomethyl) pyrrolidin-1- yl]-2-oxo-ethyl]-4- [[3-[1-(2,2- difluoroethyl)-3- (trifluoromethyl)pyrazol-4- yl]imidazo[1,2-a]pyrazin-8- yl]amino]-2-ethyl- benzamide formate | 619.6 | 620.3 |
| 528 | 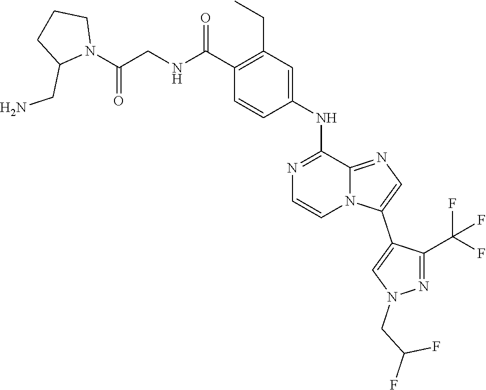 [SMILES: CCc1cc(Nc2nccn3c(-c4cn(CC(F)F)nc4C(F)(F)F)cnc23)ccc1C(=O)NCC(=O)N1CCCC1CN] | N-[2-[2-(aminomethyl) pyrrolidin-1- yl]-2-oxo-ethyl]-4- [[3-[1-(2,2- difluoroethyl)-3- (trifluoromethyl)pyrazol-4- yl]imidazo[1,2-a]pyrazin-8- yl]amino]-2-ethyl- benzamide formate | 619.6 | 620.3 |
| 529 | 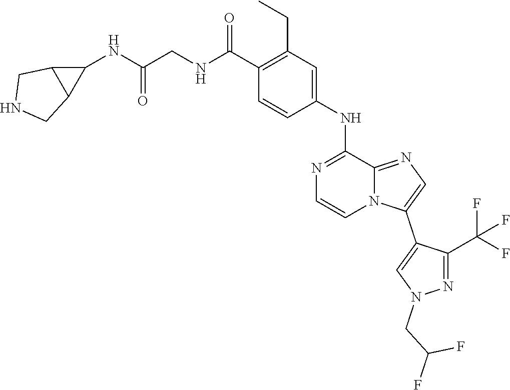 [SMILES: CCc1cc(Nc2nccn3c(-c4cn(CC(F)F)nc4C(F)(F)F)cnc23)ccc1C(=O)NCC(=O)NC1C2CNCC21] | N-[2-(3-azabicyclo[3.1.0] hexan-6- ylamino)-2-oxo-ethyl]- 4-[[3-[1- (2,2-difluoroethyl)-3- (trifluoromethyl)pyrazol-4- yl]imidazo[1,2-a]pyrazin-8- yl]amino]-2-ethyl- benzamide formate | 617.6 | 618.3 |
| 530 | 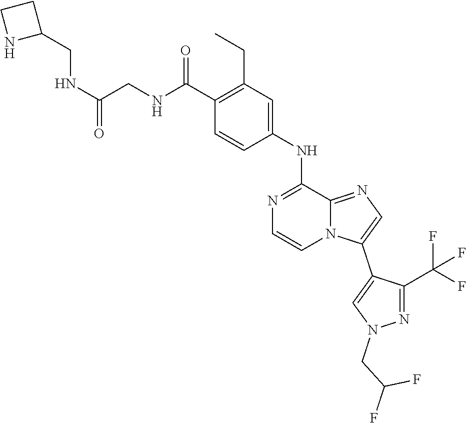 [SMILES: CCc1cc(Nc2nccn3c(-c4cn(CC(F)F)nc4C(F)(F)F)cnc23)ccc1C(=O)NCC(=O)NCC1CCN1] | N-[2-(azetidin-2- ylmethylamino)-2- oxo-ethyl]-4-[[3-[1-(2,2- difluoroethyl)-3- (trifluoromethyl)pyrazol-4- yl]imidazo[1,2-a] pyrazin-8- yl]amino]-2-ethyl- benzamide formate | 605.6 | 606.2 |
| 531 | 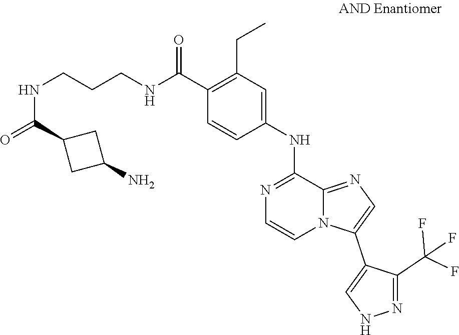 [SMILES: CCc1cc(Nc2nccn3c(-c4c[nH]nc4C(F)(F)F)cnc23)ccc1C(=O)NCCCNC(=O)[C@H]1C[C@@H](N)C1] | N-(3-((1s,3s)-3- aminocyclobutane- 1-carboxamido) propyl)-2-ethyl-4- ((3-(3-(trifluoromethyl)- 1H- pyrazol-4-yl)imidazo [1,2-a]pyrazin- 8-yl)amino)benzamide formate | 569.6 | 568.4 [M − H]− ESI neg |
| 532 | 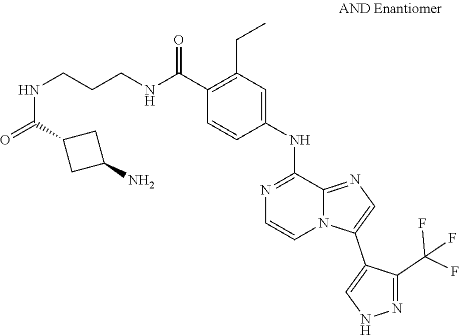 [SMILES: CCc1cc(Nc2nccn3c(-c4c[nH]nc4C(F)(F)F)cnc23)ccc1C(=O)NCCCNC(=O)[C@H]1C[C@H](N)C1] | N-(3-((1r,3r)-3- aminocyclobutane- 1-carboxamido)propyl)-2- ethyl-4- ((3-(3-(trifluoromethyl)- 1H- pyrazol-4-yl)imidazo [1,2-a]pyrazin- 8-yl)amino)benzamide formate | 569.6 | 568.4 [M − H]− ESI neg |
| 534 | 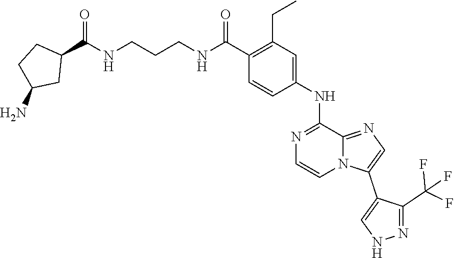 [SMILES: CCc1cc(Nc2nccn3c(-c4c[nH]nc4C(F)(F)F)cnc23)ccc1C(=O)NCCCNC(=O)[C@@H]1CC[C@H](N)C1] | N-(3-((1R,3S)-3- aminocyclopentane-1- carboxamido)propyl)-2- ethyl-4-((3- (3-(trifluoromethyl)- 1H-pyrazol-4- yl)imidazo[1,2-a]pyrazin-8- yl)amino)benzamide formate | 583.6 | 582.4 [M − H]−, ESI neg |
| 535 | 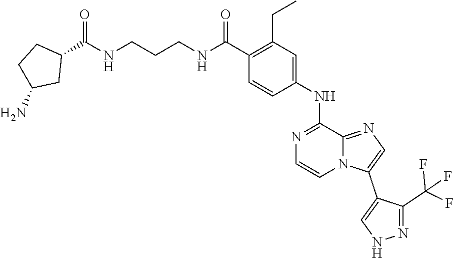 [SMILES: CCc1cc(Nc2nccn3c(-c4c[nH]nc4C(F)(F)F)cnc23)ccc1C(=O)NCCCNC(=O)[C@H]1CC[C@@H](N)C1] | N-(3-((1S,3R)-3- aminocyclopentane-1- carboxamido)propyl)- 2-ethyl-4-((3- (3-(trifluoromethyl)- 1H-pyrazol-4- yl)imidazo[1,2-a] pyrazin-8- yl)amino)benzamide formate | 583.6 | 582.4 [M − H]−, ESI neg |
| 536 | 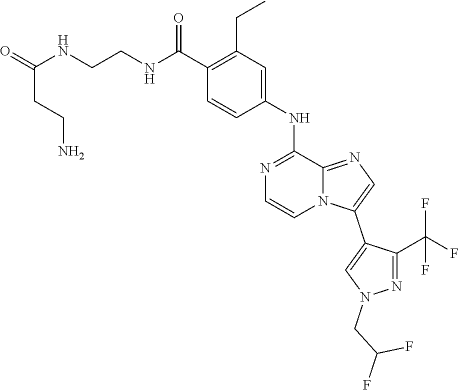 [SMILES: CCc1cc(Nc2nccn3c(-c4cn(CC(F)F)nc4C(F)(F)F)cnc23)ccc1C(=O)NCCNC(=O)CCN] | N-(2-(3- aminopropanamido) ethyl)- 4-((3-(1-(2,2- difluoroethyl)-3- (trifluoromethyl)- 1H-pyrazol-4- yl)imidazo[1,2-a] pyrazin-8- yl)amino)-2- ethylbenzamide formate | 593.6 | 594.3 |
| 537 | 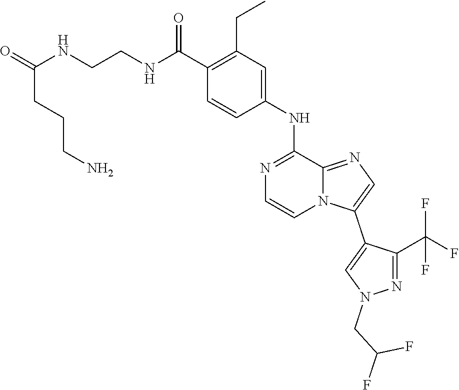 [SMILES: CCc1cc(Nc2nccn3c(-c4cn(CC(F)F)nc4C(F)(F)F)cnc23)ccc1C(=O)NCCNC(=O)CCCN] | N-(2-(4-aminobutanamido) ethyl)-4- ((3-(1-(2,2-difluoroethyl)-3- (trifluoromethyl)- 1H-pyrazol-4- yl)imidazo[1,2-a]pyrazin-8- yl)amino)-2-ethylbenzamide formate | 607.6 | 608.3 |
| 538 | 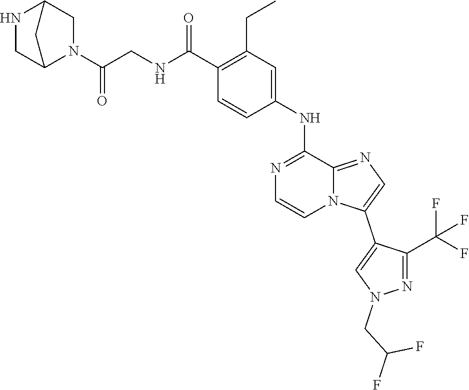 [SMILES: CCc1cc(Nc2nccn3c(-c4cn(CC(F)F)nc4C(F)(F)F)cnc23)ccc1C(=O)NCC(=O)N1CC2CC1CN2] | N-2-(2,5- diazabicyclo[2.2.1]heptan- 2-yl)-2- oxo-ethyl]-4-[[3-[1-(2,2- difluoroethyl)-3- (trifluoromethyl)pyrazol-4- yl]imidazo[1,2-a]pyrazin-8- yl]amino]-2-ethyl- benzamide formate | 617.6 | 618.2 |
| 539 | 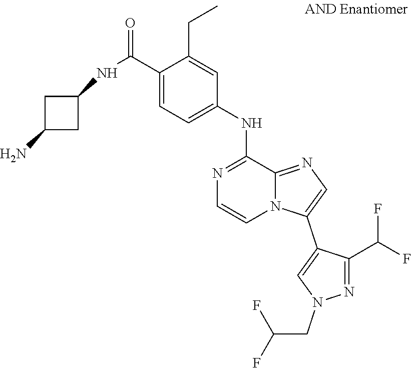 [SMILES: CCc1cc(Nc2nccn3c(-c4cn(CC(F)F)nc4C(F)F)cnc23)ccc1C(=O)N[C@H]1C[C@@H](N)C1] | N-((1s,3s)-3- aminocyclobutyl)-4- ((3-(1-(2,2-difluoroethyl)-3- (difluoromethyl)- 1H-pyrazol-4- yl)imidazo[1,2-a]pyrazin-8- yl)amino)-2-ethylbenzamide | 530.5 | 531.3 |
| 540 | 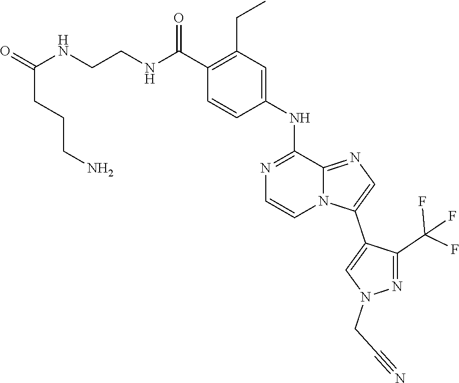 [SMILES: CCc1cc(Nc2nccn3c(-c4cn(CC#N)nc4C(F)(F)F)cnc23)ccc1C(=O)NCCNC(=O)CCCN] | N-(2-(4-aminobutanamido) ethyl)-4- ((3-(1-(cyanomethyl)-3- (trifluoromethyl)-1H- pyrazol-4- yl)imidazo[1,2-a]pyrazin-8- yl)amino)-2-ethylbenzamide formate | 582.6 | 583.2 |
| 541 | 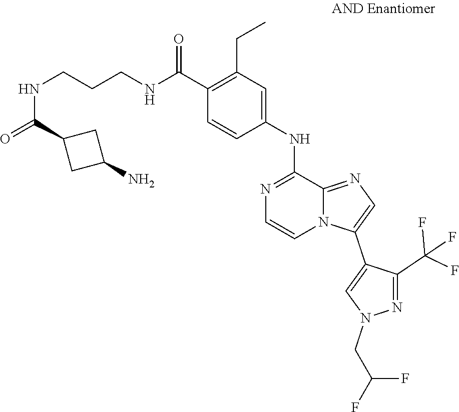 [SMILES: CCc1cc(Nc2nccn3c(-c4cn(CC(F)F)nc4C(F)(F)F)cnc23)ccc1C(=O)NCCCNC(=O)[C@H]1C[C@@H](N)C1] | N-(3-((1s,3s)-3- aminocyclobutane- 1-carboxamido) propyl)-4-((3-(1- (2,2-difluoroethyl)-3- (trifluoromethyl)- 1H-pyrazol-4- yl)imidazo[1,2-a] pyrazin-8- yl)amino)-2- ethylbenzamide formate | 633.6 | 678.5 [M + HCOO]−, ESI neg, |
| 542 | 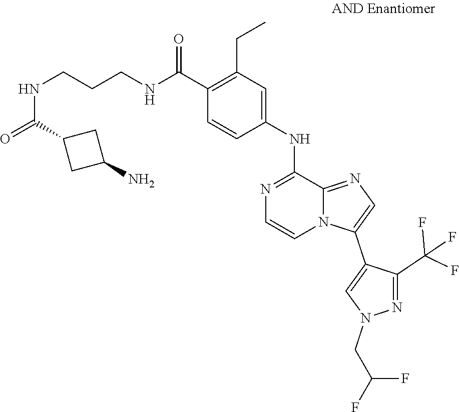 [SMILES: CCc1cc(Nc2nccn3c(-c4cn(CC(F)F)nc4C(F)(F)F)cnc23)ccc1C(=O)NCCCNC(=O)[C@H]1C[C@H](N)C1] | N-(3-((1r,3r)-3- aminocyclobutane- 1-carboxamido) propyl)-4-((3-(1- (2,2-difluoroethyl)-3- (trifluoromethyl)- 1H-pyrazol-4- yl)imidazo[1,2-a]pyrazin-8- yl)amino)-2-ethylbenzamide formate | 633.6 | 678.5 [M + HCOO]−, ESI neg |
| 543 | 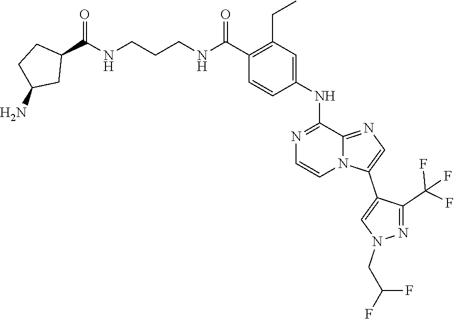 [SMILES: CCc1cc(Nc2nccn3c(-c4cn(CC(F)F)nc4C(F)(F)F)cnc23)ccc1C(=O)NCCCNC(=O)[C@@H]1CC[C@H](N)C1] | N-(3-((1R,3S)-3- aminocyclopentane-1- carboxamido)propyl)- 4-((3-(1-(2,2- difluoroethyl)-3- (trifluoromethyl)- 1H-pyrazol-4-yl) imidazo[1,2- a]pyrazin-8-yl)amino)-2- ethylbenzamide formate | 647.7 | 692.5 [M + HCOO]−, ESI neg |
| 544 | 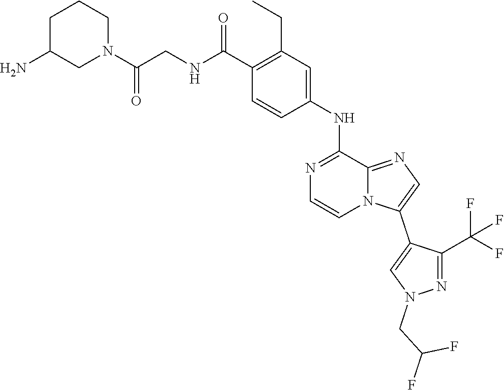 [SMILES: CCc1cc(Nc2nccn3c(-c4cn(CC(F)F)nc4C(F)(F)F)cnc23)ccc1C(=O)NCC(=O)N1CCCC(N)C1] | N-[2-(3-amino-1- piperidyl)-2-oxo- ethyl]-4-[[3-[1-(2,2- difluoroethyl)- 3-(trifluoromethyl) pyrazol-4- yl]imidazo[1,2-a]pyrazin- 8-yl]amino]-2-ethyl- benzamide formate | 619.6 | 620.2 |
| 545 | 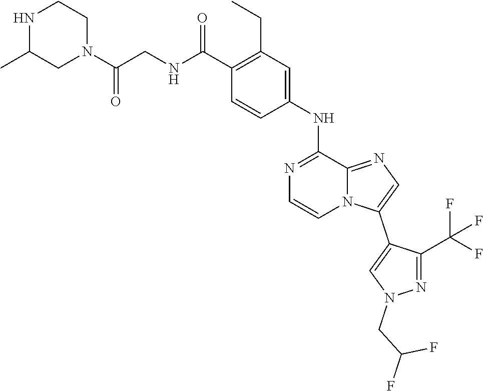 [SMILES: CCc1cc(Nc2nccn3c(-c4cn(CC(F)F)nc4C(F)(F)F)cnc23)ccc1C(=O)NCC(=O)N1CCNC(C)C1] | 4-[[3-[1-(2,2- difluoroethyl)-3- (trifluoromethyl)pyrazol-4- yl]imidazo[1,2-a]pyrazin-8- yl]amino]-2-ethyl-N-[2-(3- methylpiperazin-1-yl)-2-oxo- ethyl]benzamide formate | 619.6 | 620.3 |
| 546 | 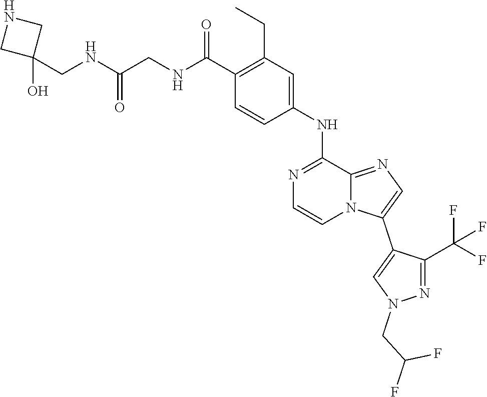 [SMILES: CCc1cc(Nc2nccn3c(-c4cn(CC(F)F)nc4C(F)(F)F)cnc23)ccc1C(=O)NCC(=O)NCC1(O)CNC1] | 4-[[3-[1-(2,2- difluoroethyl)-3- (trifluoromethyl)pyrazol-4- yl]imidazo[1,2-a]pyrazin-8- yl]amino]-2-ethyl-N-[2-[(3- hydroxyazetidin-3- yl)methylamino]-2-oxo- ethyl]benzamide formate | 621.6 | 622.2 |
| 547 | 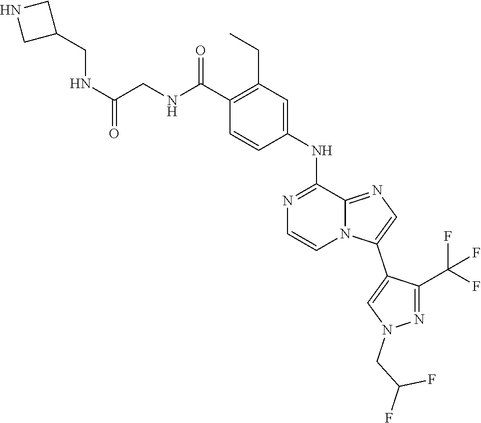 [SMILES: CCc1cc(Nc2nccn3c(-c4cn(CC(F)F)nc4C(F)(F)F)cnc23)ccc1C(=O)NCC(=O)NCC1CNC1] | N-[2-(azetidin-3- ylmethylamino)-2- oxo-ethyl]-4-[[3-[1-(2,2- difluoroethyl)-3- (trifluoromethyl)pyrazol-4- yl]imidazo[1,2-a]pyrazin-8- yl]amino]-2-ethyl- benzamide formate | 605.6 | 606.2 |
| 548 | 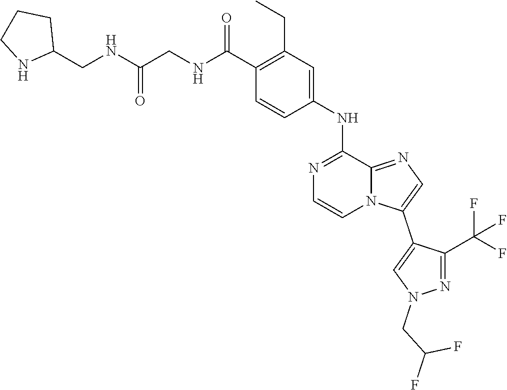 [SMILES: CCc1cc(Nc2nccn3c(-c4cn(CC(F)F)nc4C(F)(F)F)cnc23)ccc1C(=O)NCC(=O)NCC1CCCN1] | 4-[[3-[1-(2,2- difluoroethyl)-3- (trifluoromethyl)pyrazol-4- yl]imidazo[1,2-a]pyrazin-8- yl]amino]-2-ethyl- N-[2-oxo-2- (pyrrolidin-2- ylmethylamino)ethyl] benzamide formate | 619.6 | 620.2 |
| 549 | 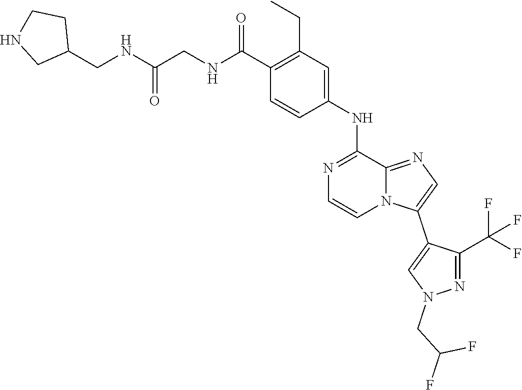 [SMILES: CCc1cc(Nc2nccn3c(-c4cn(CC(F)F)nc4C(F)(F)F)cnc23)ccc1C(=O)NCC(=O)NCC1CCNC1] | 4-[[3-[1-(2,2- difluoroethyl)-3- (trifluoromethyl)pyrazol-4- yl]imidazo[1,2-a]pyrazin-8- yl]amino]-2-ethyl- N-[2-oxo-2- (pyrrolidin-3- ylmethylamino)ethyl] benzamide formate | 619.6 | 620.2 |
| 550 | 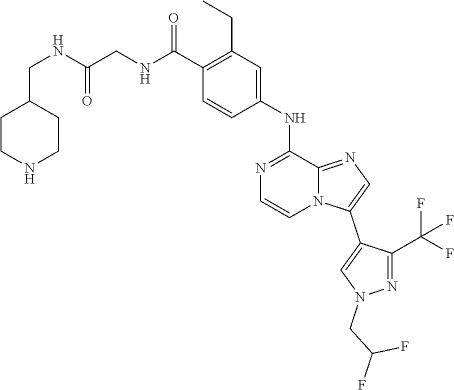 [SMILES: CCc1cc(Nc2nccn3c(-c4cn(CC(F)F)nc4C(F)(F)F)cnc23)ccc1C(=O)NCC(=O)NCC1CCNCC1] | 4-[[3-[1-(2,2- difluoroethyl)-3- (trifluoromethyl)pyrazol-4- yl]imidazo[1,2-a]pyrazin-8- yl]amino]-2-ethyl-N- [2-oxo-2-(4- piperidylmethylamino) ethyl] benzamide formate | 633.6 | 634.3 |
| 551 | 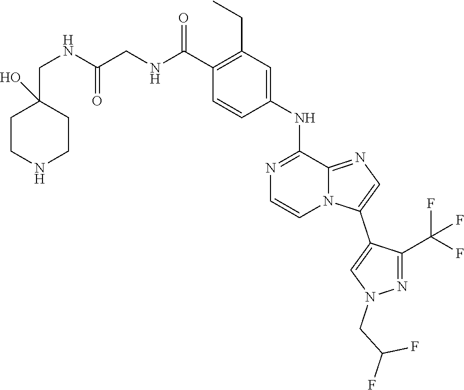 [SMILES: CCc1cc(Nc2nccn3c(-c4cn(CC(F)F)nc4C(F)(F)F)cnc23)ccc1C(=O)NCC(=O)NCC1(O)CCNCC1] | 4-[[3-[1-(2,2- difluoroethyl)-3- (trifluoromethyl)pyrazol-4- yl]imidazo[1,2-a]pyrazin-8- yl]amino]-2-ethyl-N-[2-[(4- hydroxy-4-piperidyl) methylamino]- 2-oxo-ethyl]benzamide formate | 649.6 | 650.2 |
| 552 | 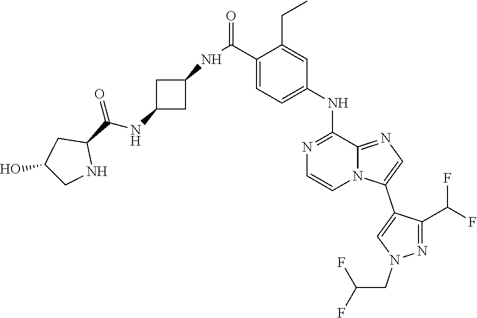 [SMILES: CCc1cc(Nc2nccn3c(-c4cn(CC(F)F)nc4C(F)F)cnc23)ccc1C(=O)N[C@H]1C[C@@H](NC(=O)[C@@H]2C[C@@H](O)CN2)C1] | (2S,4R)-N-((1s,3R)- 3-(4-((3-(1- (2,2-difluoroethyl)-3- (difluoromethyl)- 1H-pyrazol-4- yl)imidazo[1,2-a]pyrazin-8- yl)amino)-2- ethylbenzamido) cyclobutyl)-4- hydroxypyrrolidine- 2-carboxamide | 643.6 | 644.4 |
| 553 | 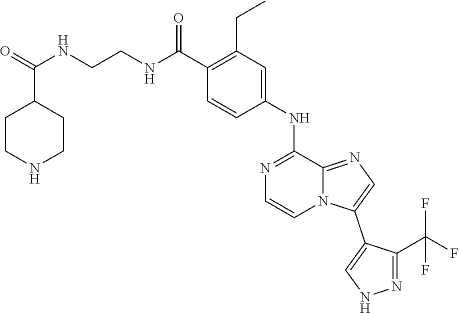 [SMILES: CCc1cc(Nc2nccn3c(-c4c[nH]nc4C(F)(F)F)cnc23)ccc1C(=O)NCCNC(=O)C1CCNCC1] | N-(2-(2-ethyl-4-((3-(3- (trifluoromethyl)- 1H-pyrazol-4- yl)imidazo[1,2-a]pyrazin-8- yl)amino)benzamido)ethyl) piperidine-4-carboxamide formate | 569.6 | 570.3 |
| 554 | 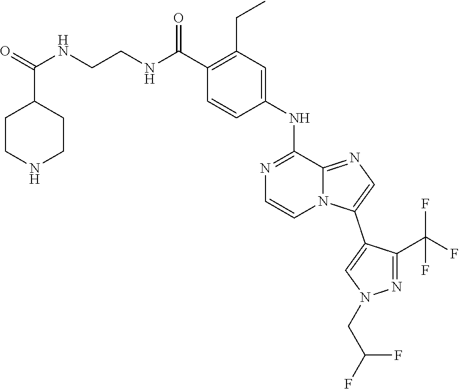 [SMILES: CCc1cc(Nc2nccn3c(-c4cn(CC(F)F)nc4C(F)(F)F)cnc23)ccc1C(=O)NCCNC(=O)C1CCNCC1] | N-(2-(4-((3-(1-(2,2- difluoroethyl)- 3-(trifluoromethyl)- 1H-pyrazol-4- yl)imidazo[1,2-a]pyrazin- 8-yl)amino)-2- ethylbenzamido)ethyl) piperidine-4- carboxamide formate | 633.6 | 634.4 |
| 555 | 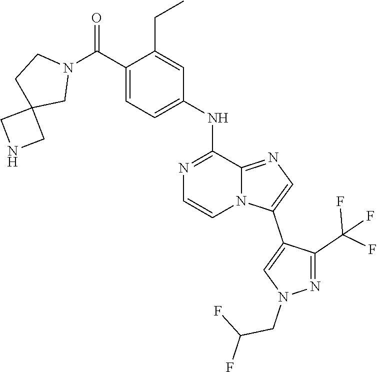 [SMILES: CCc1cc(Nc2nccn3c(-c4cn(CC(F)F)nc4C(F)(F)F)cnc23)ccc1C(=O)N1CCC2(CNC2)C1] | (4-((3-(1-(2,2- difluoroethyl)-3- (trifluoromethyl)- 1H-pyrazol-4- yl)imidazo[1,2-a] pyrazin-8- yl)amino)-2- ethylphenyl)(2,6- diazaspiro[3.4]octan-6- yl)methanone formate | 574.6 | 575.2 |
| 556 | 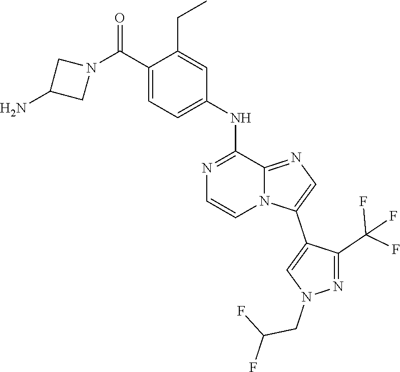 [SMILES: CCc1cc(Nc2nccn3c(-c4cn(CC(F)F)nc4C(F)(F)F)cnc23)ccc1C(=O)N1CC(N)C1] | (3-aminoazetidin-1-yl)(4- ((3-(1- (2,2-difluoroethyl)-3- (trifluoromethyl)- 1H-pyrazol-4- yl)imidazo[1,2-a]pyrazin- 8-yl)amino)-2- ethylphenyl)methanone formate | 534.5 | 535.2 |
| 557 | 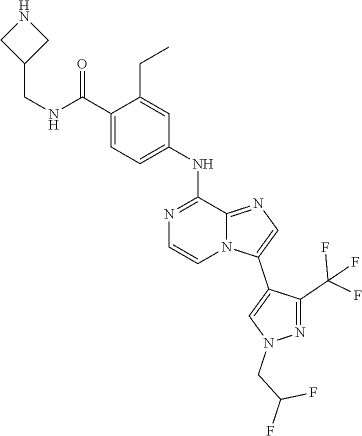 [SMILES: CCc1cc(Nc2nccn3c(-c4cn(CC(F)F)nc4C(F)(F)F)cnc23)ccc1C(=O)NCC1CNC1] | N-(azetidin-3-ylmethyl)- 4-((3-(1- (2,2-difluoroethyl)-3- (trifluoromethyl)- 1H-pyrazol-4- yl)imidazo[1,2-a] pyrazin-8- yl)amino)-2- ethylbenzamide formate | 548.5 | 549.3 |
| 558 | 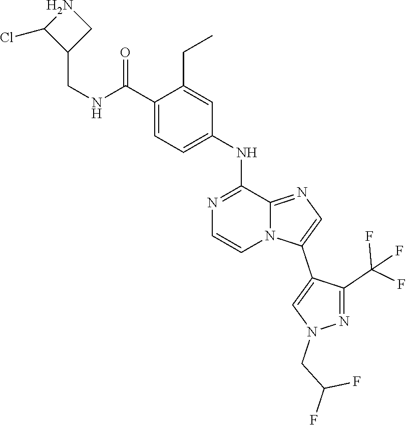 [SMILES: CCc1cc(Nc2nccn3c(-c4cn(CC(F)F)nc4C(F)(F)F)cnc23)ccc1C(=O)NCC1CNC1Cl] | N-(3-amino-2- (chloromethyl) propyl)-4-((3-(1- (2,2-difluoroethyl)-3- (trifluoromethyl)- 1H-pyrazol-4- yl)imidazo[1,2-a] pyrazin-8- yl)amino)-2- ethylbenzamide formate | 585.0 | 585.2 |
| 559 | 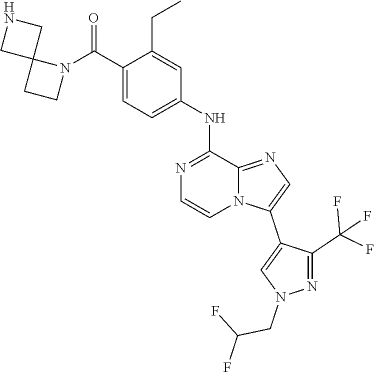 [SMILES: CCc1cc(Nc2nccn3c(-c4cn(CC(F)F)nc4C(F)(F)F)cnc23)ccc1C(=O)N1CCC12CNC2] | (4-((3-(1-(2,2- difluoroethyl)-3- (trifluoromethyl)- 1H-pyrazol-4- yl)imidazo[1,2-a]pyrazin- 8-yl)amino)-2- ethylphenyl)(1,6- diazaspiro[3.3]heptan-1- yl)methanone formate | 560.5 | 561.2 |
| 560 | 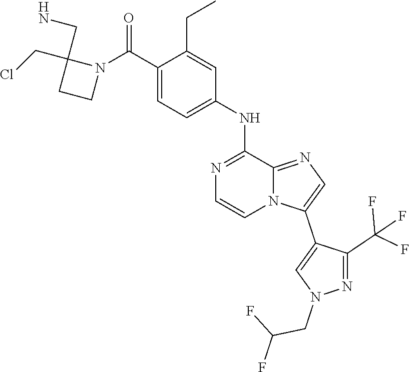 | (2-(aminomethyl)-2- (chloromethyl)azetidin- 1-yl)(4-((3- (1-(2,2-difluoroethyl)-3- (trifluoromethyl)- 1H-pyrazol-4- yl)imidazo[1,2-a]pyrazin- 8-yl)amino)-2- ethylphenyl) methanone formate | 597.0 | 597.2 |
| 561 | 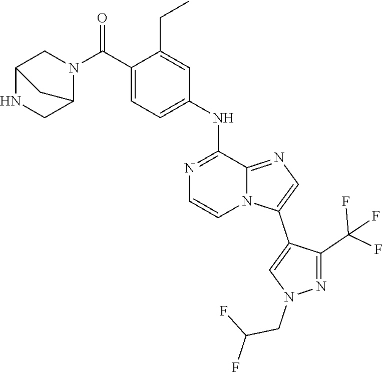 [SMILES: CCc1cc(Nc2nccn3c(-c4cn(CC(F)F)nc4C(F)(F)F)cnc23)ccc1C(=O)N1CC2CC1CN2] | (2,5-diazabicyclo [2.2.1]heptan-2- yl)(4-((3-(1(2,2- difluoroethyl)-3- (trifluoromethyl)- 1H-pyrazol-4- yl)imidazo[1,2-a] pyrazin-8- yl)amino)-2- ethylphenyl) methanone formate | 560.5 | 561.2 |
| 562 | 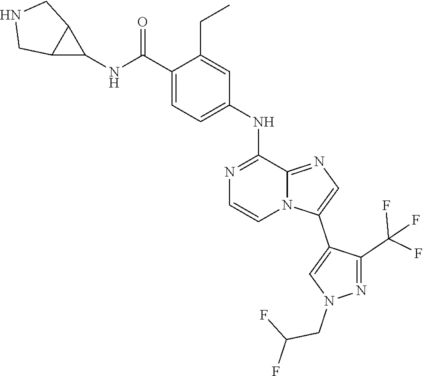 [SMILES: CCc1cc(Nc2nccn3c(-c4cn(CC(F)F)nc4C(F)(F)F)cnc23)ccc1C(=O)NC1C2CNCC21] | N-(3-azabicyclo[3.1.0] hexan-6-yl)- 4-((3-(1-(2,2- difluoroethyl)-3- (trifluoromethyl)- 1H-pyrazol-4- yl)imidazo[1,2-a] pyrazin-8- yl)amino)-2- ethylbenzamide formate | 560.5 | 561.3 |
| 563 | 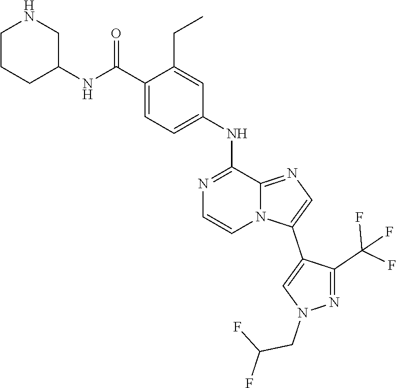 [SMILES: CCc1cc(Nc2nccn3c(-c4cn(CC(F)F)nc4C(F)(F)F)cnc23)ccc1C(=O)NC1CCCNC1] | 4-((3-(1-(2,2- difluoroethyl)-3- (trifluoromethyl)- 1H-pyrazol-4- yl)imidazo[1,2-a] pyrazin-8- yl)amino)-2-ethyl- N-(piperidin-3- yl)benzamide formate | 562.5 | 563.3 |
| 564 | 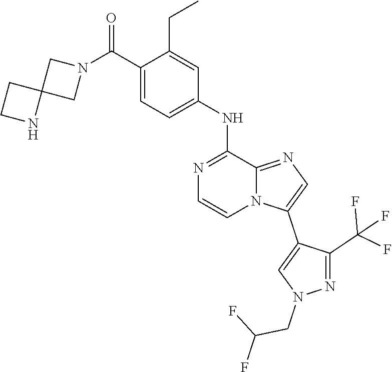 [SMILES: CCc1cc(Nc2nccn3c(-c4cn(CC(F)F)nc4C(F)(F)F)cnc23)ccc1C(=O)N1CC2(CCN2)C1] | (4-((3-(1-(2,2- difluoroethyl)-3- (trifluoromethyl)- 1H-pyrazol-4- yl)imidazo[1,2-a] pyrazin-8- yl)amino)-2- ethylphenyl)(1,6- diazaspiro[3.3]heptan-6- yl)methanone formate | 560.5 | 561.2 |
| 565 | 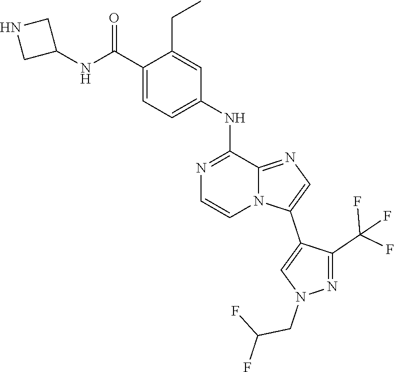 [SMILES: CCc1cc(Nc2nccn3c(-c4cn(CC(F)F)nc4C(F)(F)F)cnc23)ccc1C(=O)NC1CNC1] | N-(azetidin-3-yl)-4- ((3-(1-(2,2- difluoroethyl)-3- (trifluoromethyl)- 1H-pyrazol-4-yl) imidazo[1,2- a]pyrazin-8-yl)amino)-2- ethylbenzamide formate | 534.5 | 535.2 |
| 566 | 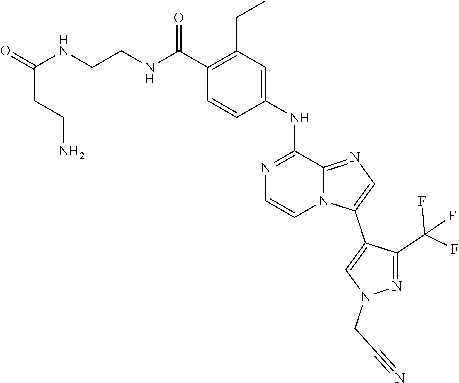 [SMILES: CCc1cc(Nc2nccn3c(-c4cn(CC#N)nc4C(F)(F)F)cnc23)ccc1C(=O)NCCNC(=O)CCN] | N-(2-(3- aminopropanamido) ethyl)- 4-((3-(1-(cyanomethyl)-3- (trifluoromethyl)- 1H-pyrazol-4- yl)imidazo[1,2-a] pyrazin-8- yl)amino)-2- ethylbenzamide formate | 568.6 | 569.3 |
| 567 | 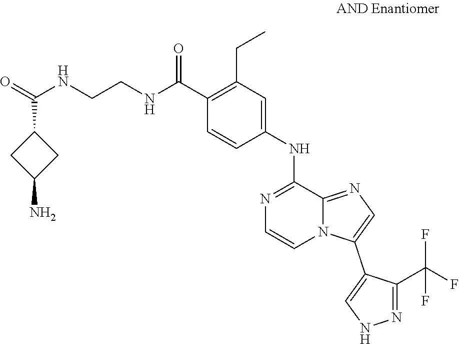 [SMILES: CCc1cc(Nc2nccn3c(-c4c[nH]nc4C(F)(F)F)cnc23)ccc1C(=O)NCCNC(=O)[C@H]1C[C@H](N)C1] | N-(2-((1r,3r)-3- aminocyclobutane- 1-carboxamido)ethyl)-2- ethyl-4-((3- (3-(trifluoromethyl)- 1H-pyrazol-4- yl)imidazo[1,2-a] pyrazin-8- yl)amino) benzamide formate | 555.6 | 556.3 |
| 568 | 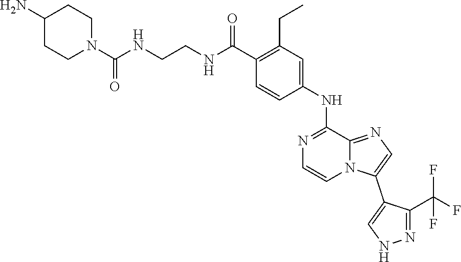 [SMILES: CCc1cc(Nc2nccn3c(-c4c[nH]nc4C(F)(F)F)cnc23)ccc1C(=O)NCCNC(=O)N1CCC(N)CC1] | 4-amino-N-(2-(2- ethyl-4-((3-(3- (trifluoromethyl)- 1H-pyrazol-4- yl)imidazo[1,2-a]pyrazin-8- yl)amino)benzamido)ethyl) piperidine-1-carboxamide formate | 584.6 | 585.3 |
| 569 | 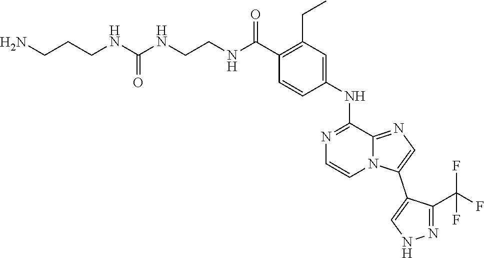 [SMILES: CCc1cc(Nc2nccn3c(-c4c[nH]nc4C(F)(F)F)cnc23)ccc1C(=O)NCCNC(=O)NCCCN] | N-(2-(3-(3- aminopropyl)ureido) ethyl)-2-ethyl- 4-((3-(3- (trifluoromethyl)-1H- pyrazol-4-yl)imidazo[1,2-a] pyrazin- 8-yl)amino)benzamide formate | 558.6 | 559.3 |
| 570 | 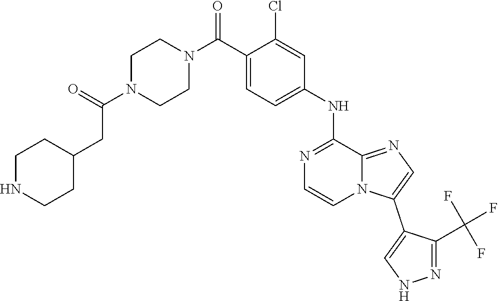 [SMILES: O=C(CC1CCNCC1)N1CCN(C(=O)c2ccc(Nc3nccn4c(-c5c[nH]nc5C(F)(F)F)cnc34)cc2Cl)CC1] | 1-(4-(2-chloro-4-((3-(3- (trifluoromethyl)- 1H-pyrazol-4- yl)imidazo[1,2-a]pyrazin-8- yl)amino)benzoyl) piperazin-1-yl)- 2-(piperidin-4-yl) ethan-1-one formate | 616.0 | 614.4 [M − H]−, ESI neg |
| 571 | 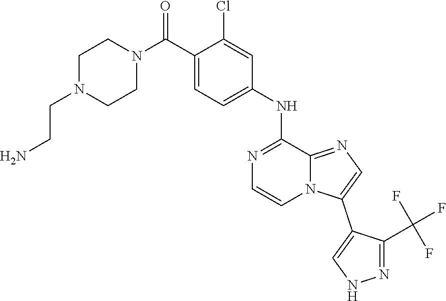 [SMILES: NCCN1CCN(C(=O)c2ccc(Nc3nccn4c(-c5c[nH]nc5C(F)(F)F)cnc34)cc2Cl)CC1] | (4-(2-aminoethyl)piperazin- 1-yl)(2- chloro-4-((3-(3- (trifluoromethyl)- 1H-pyrazol-4-yl) imidazo[1,2- a]pyrazin-8- yl)amino)phenyl) methanone formate | 533.9 | 532.3 [M − H]−, ESI neg |
| 572 | 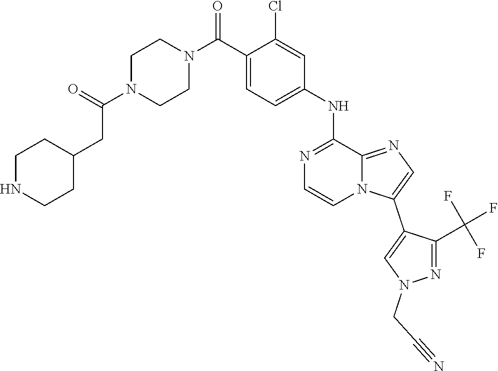 [SMILES: N#CCn1cc(-c2cnc3c(Nc4ccc(C(=O)N5CCN(C(=O)CC6CCNCC6)CC5)c(Cl)c4)nccn23)c(C(F)(F)F)n1] | 2-(4-(8-((3-chloro-4-(4-(2- (piperidin-4-yl)acetyl) piperazine-1- carbonyl)phenyl)amino) imidazo[1,2- a]pyrazin-3-yl)-3- (trifluoromethyl)- 1H-pyrazol-1- yl)acetonitrile formate | 655.1 | 699.5 [M + HCOO]−, ESI neg |
| 573 | 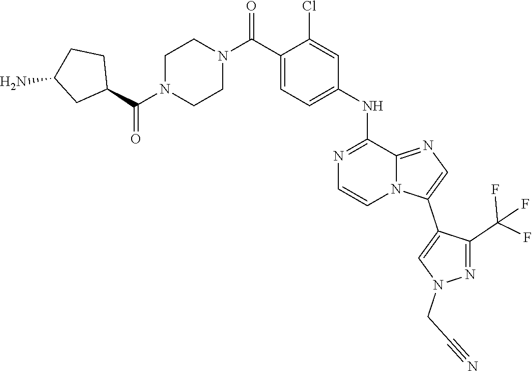 [SMILES: N#CCn1cc(-c2cnc3c(Nc4ccc(C(=O)N5CCN(C(=O)[C@@H]6CC[C@@H](N)C6)CC5)c(Cl)c4)nccn23)c(C(F)(F)F)n1] | 2-(4-(8-((4-(4-((1R,3R)-3- aminocyclopentane-1- carbonyl)piperazine-1- carbonyl)-3- chlorophenyl)amino) imidazo[1,2- a]pyrazin-3-yl)-3- (trifluoromethyl)- 1H-pyrazol-1-yl)acetonitrile formate | 641.1 | 685.4 [M + HCOO]−, ESI neg |
| 574 | 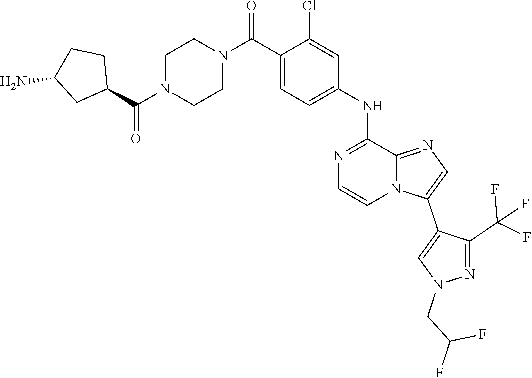 [SMILES: N[C@@H]1CC[C@@H](C(=O)N2CCN(C(=O)c3ccc(Nc4nccn5c(-c6cn(CC(F)F)nc6C(F)(F)F)cnc45)cc3Cl)CC2)C1] | (4-((1R,3R)-3- aminocyclopentane- 1-carbonyl)piperazin-1-yl)(2- chloro-4-((3-(1-(2,2- difluoroethyl)- 3-(trifluoromethyl)- 1H-pyrazol-4- yl)imidazo[1,2-a]pyrazin-8- yl)amino)phenyl)methanone formate | 666.1 | 710.5 [M + HCOO]−, ESI neg |
| 575 | 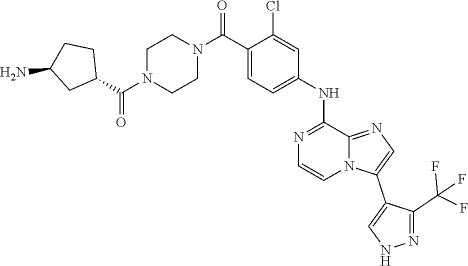 [SMILES: N[C@H]1CC[C@H](C(=O)N2CCN(C(=O)c3ccc(Nc4nccn5c(-c6c[nH]nc6C(F)(F)F)cnc45)cc3Cl)CC2)C1] | (4-((1S,3S)-3- aminocyclopentane- 1-carbonyl)piperazin- 1-yl)(2- chloro-4-((3-(3- (trifluoromethyl)- 1H-pyrazol-4-yl) imidazo[1,2- a]pyrazin-8- yl)amino)phenyl)methanone formate | 602.0 | 600.3 [M − H]−, ESI neg |
| 576 | 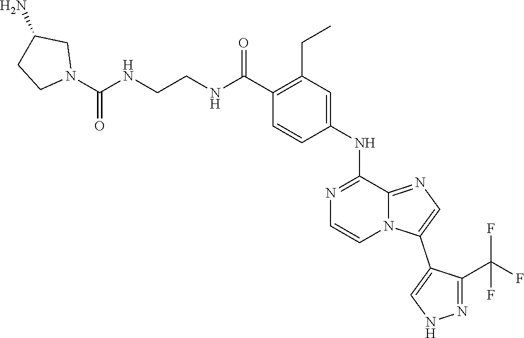 [SMILES: CCc1cc(Nc2nccn3c(-c4c[nH]nc4C(F)(F)F)cnc23)ccc1C(=O)NCCNC(=O)N1CC[C@H](N)C1] | (S)-3-amino-N-(2-(2- ethyl-4-((3-(3- (trifluoromethyl)- 1H-pyrazol-4- yl)imidazo[1,2-a]pyrazin-8- yl)amino)benzamido)ethyl) pyrrolidine-1-carboxamide formate | 570.6 | 571.3 |
| 577 | 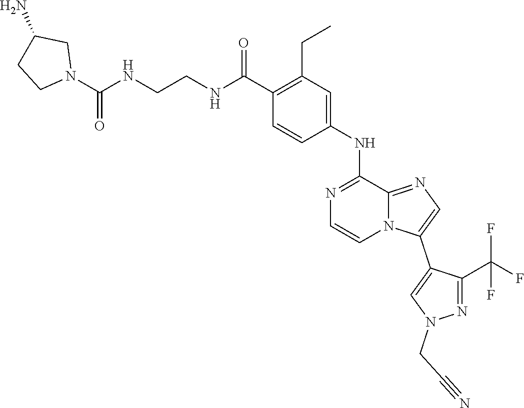 [SMILES: CCc1cc(Nc2nccn3c(-c4cn(CC#N)nc4C(F)(F)F)cnc23)ccc1C(=O)NCCNC(=O)N1CC[C@H](N)C1] | (S)-3-amino-N-(2-(4-((3-(1- (cyanomethyl)-3- (trifluoromethyl)- 1H-pyrazol-4-yl) imidazo[1,2- a]pyrazin-8-yl)amino)-2- ethylbenzamido)ethyl) pyrrolidine- 1-carboxamide formate | 609.6 | 610.3 |
| 578 | 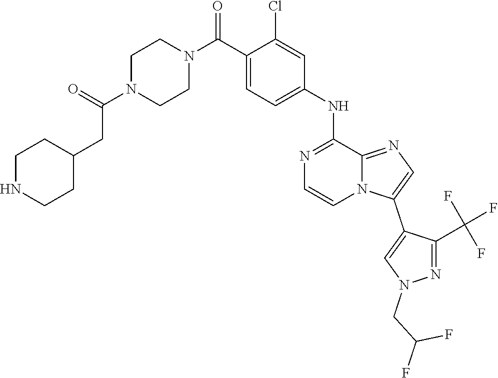 [SMILES: O=C(CC1CCNCC1)N1CCN(C(=O)c2ccc(Nc3nccn4c(-c5cn(CC(F)F)nc5C(F)(F)F)cnc34)cc2Cl)CC1] | 1-(4-(2-chloro-4-((3- (1-(2,2- difluoroethyl)-3- (trifluoromethyl)- 1H-pyrazol-4-yl) imidazo[1,2- a]pyrazin-8- yl)amino)benzoyl) piperazin-1-yl)- 2-(piperidin-4-yl) ethan-1-one formate | 680.1 | 724.4 [M + HCOO]−, ESI neg |
| 579 | 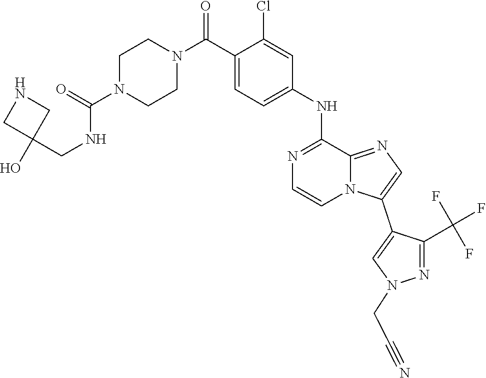 [SMILES: N#CCn1cc(-c2cnc3c(Nc4ccc(C(=O)N5CCN(C(=O)NCC6(O)CNC6)CC5)c(Cl)c4)nccn23)c(C(F)(F)F)n1] | 4-(2-chloro-4-((3-(1- (cyanomethyl)- 3-(trifluoromethyl)-1H- pyrazol-4- yl)imidazo[1,2-a]pyrazin-8- yl)amino)benzoyl)-N-((3- hydroxyazetidin-3- yl)methyl)piperazine-1- carboxamide formate | 658.0 | 702.4 [M + HCOO]−, ESI neg |
| 580 | 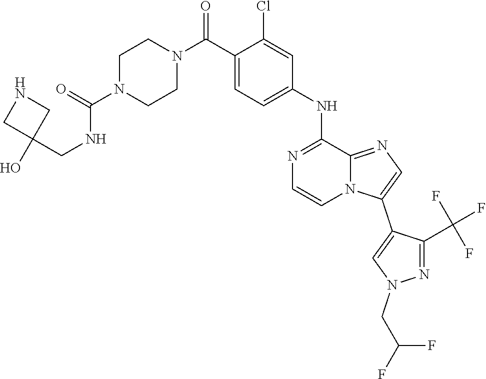 [SMILES: O=C(NCC1(O)CNC1)N1CCN(C(=O)c2ccc(Nc3nccn4c(-c5cn(CC(F)F)nc5C(F)(F)F)cnc34)cc2Cl)CC1] | 4-(2-chloro-4-((3-(1-(2,2- difluoroethyl)-3- (trifluoromethyl)- 1H-pyrazol-4-yl) imidazo[1,2- a]pyrazin-8-yl)amino) benzoyl)-N- ((3-hydroxyazetidin-3- yl)methyl)piperazine-1- carboxamide formate | 683.0 | 727.4 [M + HCOO]−, ESI neg |
| 581 | 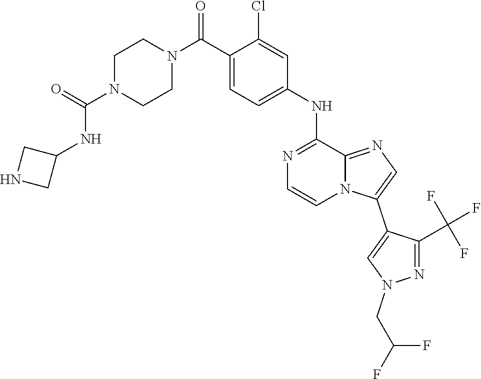 [SMILES: O=C(NC1CNC1)N1CCN(C(=O)c2ccc(Nc3nccn4c(-c5cn(CC(F)F)nc5C(F)(F)F)cnc34)cc2Cl)CC1] | N-(azetidin-3-yl)-4- (2-chloro-4-((3- (1-(2,2-difluoroethyl)-3- (trifluoromethyl)- 1H-pyrazol-4- yl)imidazo[1,2-a] pyrazin-8- yl)amino)benzoyl) piperazine-1- carboxamide formate | 653.0 | 697.4 [M + HCOO]−, ESI neg |
| 582 | 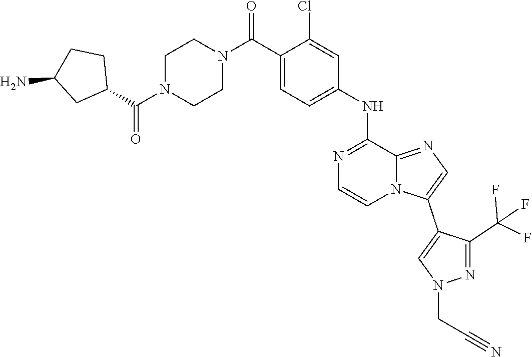 [SMILES: N#CCn1cc(-c2cnc3c(Nc4ccc(C(=O)N5CCN(C(=O)[C@H]6CC[C@H](N)C6)CC5)c(Cl)c4)nccn23)c(C(F)(F)F)n1] | 2-(4-(8-((4-(4-((1S,3S)-3- aminocyclopentane-1- carbonyl)piperazine- 1-carbonyl)-3- chlorophenyl)amino) imidazo[1,2- a]pyrazin-3-yl)-3- (trifluoromethyl)- 1H-pyrazol-1-yl) acetonitrile formate | 641.1 | 685.4 [M + HCOO]−, ESI neg |
| 583 | 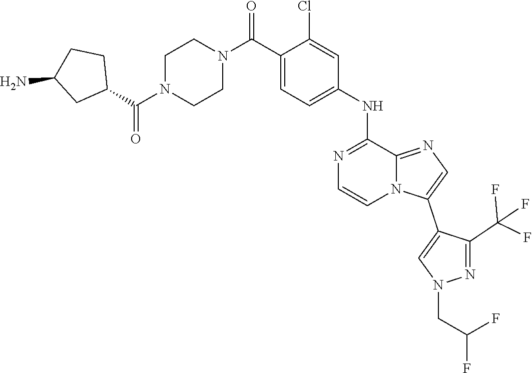 [SMILES: N[C@H]1CC[C@H](C(=O)N2CCN(C(=O)c3ccc(Nc4nccn5c(-c6cn(CC(F)F)nc6C(F)(F)F)cnc45)cc3Cl)CC2)C1] | (4-((1S,3S)-3- aminocyclopentane- 1-carbonyl)piperazin-1-yl) (2-chloro-4-((3-(1-(2,2- difluoroethyl)- 3-(trifluoromethyl)- 1H-pyrazol-4- yl)imidazo[1,2-a] pyrazin-8- yl)amino)phenyl) methanone formate | 666.1 | 710.5 [M + HCOO]−, ESI neg |
| 584 | 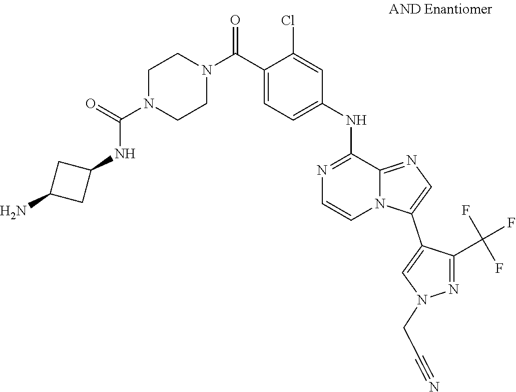 [SMILES: N#CCn1cc(-c2cnc3c(Nc4ccc(C(=O)N5CCN(C(=O)N[C@H]6C[C@@H](N)C6)CC5)c(Cl)c4)nccn23)c(C(F)(F)F)n1] | N-((1s,3s)-3- aminocyclobutyl)-4- (2-chloro-4-((3-(1- (cyanomethyl)-3- (trifluoromethyl)- 1H-pyrazol-4- yl)imidazo[1,2-a] pyrazin-8- yl)amino)benzoyl) piperazine-1- carboxamide formate | 642.0 | 686.4 [M + HCOO]−, ESI neg |
| 585 | 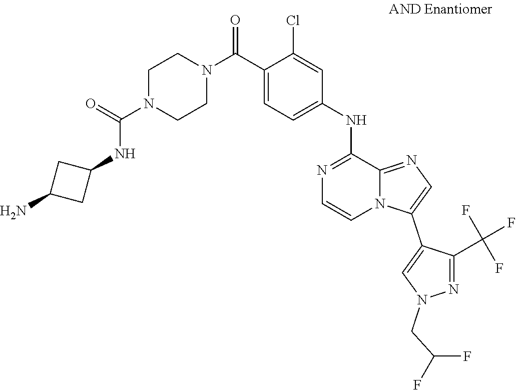 [SMILES: N[C@H]1C[C@@H](NC(=O)N2CCN(C(=O)c3ccc(Nc4nccn5c(-c6cn(CC(F)F)nc6C(F)(F)F)cnc45)cc3Cl)CC2)C1] | N-((1s,3s)-3- aminocyclobutyl)-4- (2-chloro-4-((3-(1-(2,2- difluoroethyl)-3- (trifluoromethyl)- 1H-pyrazol-4-yl) imidazo[1,2- a]pyrazin-8- yl)amino)benzoyl) piperazine-1- carboxamide formate | 667.0 | 711.4 [M + HCOO]−, ESI neg |
| 586 | 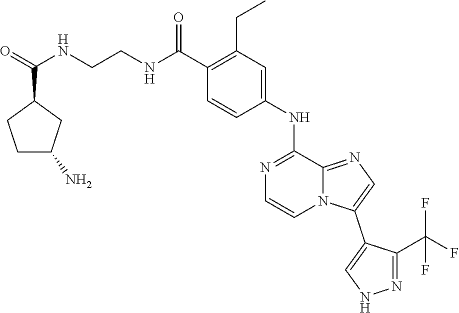 [SMILES: CCc1cc(Nc2nccn3c(-c4c[nH]nc4C(F)(F)F)cnc23)ccc1C(=O)NCCNC(=O)[C@@H]1CC[C@@H](N)C1] | N-[2-[[(1R,3R)-3- aminocyclo- pentanecarbonyl] amino] ethyl]-2-ethyl-4-[[3-[3- (trifluoromethyl)- 1H-pyrazol-4- yl]imidazo[1,2-a] pyrazin-8- yl]amino]benzamide formate | 569.6 | 568.4 [M − H]−, ESI neg |
| 587 | 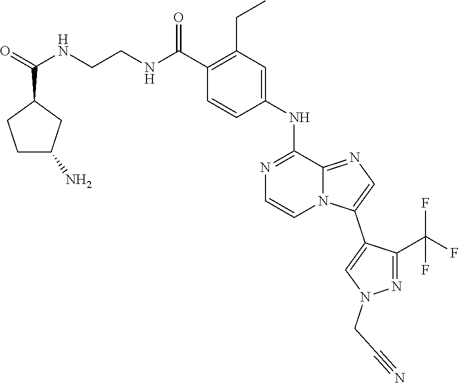 [SMILES: CCc1cc(Nc2nccn3c(-c4cn(CC#N)nc4C(F)(F)F)cnc23)ccc1C(=O)NCCNC(=O)[C@@H]1CC[C@@H](N)C1] | N-(2-((1R,3R)-3- aminocyclopentane-1- carboxamido)ethyl)- 4-((3-(1- (cyanomethyl)-3- (trifluoromethyl)- 1H-pyrazol-4-yl) imidazo[1,2- a]pyrazin-8-yl)amino)-2- ethylbenzamide formate | 608.6 | 607.4 [M − H]−, ESI neg |
| 588 | 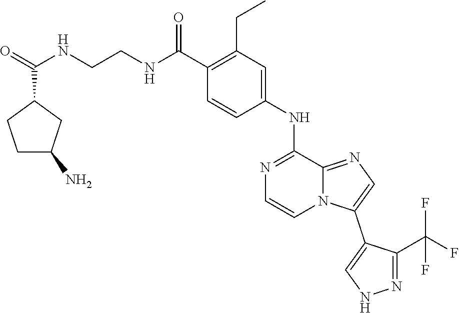 [SMILES: CCc1cc(Nc2nccn3c(-c4c[nH]nc4C(F)(F)F)cnc23)ccc1C(=O)NCCNC(=O)[C@H]1CC[C@H](N)C1] | N-(2-((1S,3S)-3- aminocyclopentane-1- carboxamido)ethyl)- 2-ethyl-4-((3- (3-(trifluoromethyl)- 1H-pyrazol-4- yl)imidazo[1,2-a] pyrazin-8- yl)amino)benzamide formate | 569.6 | 568.4 [M − H]−, ESI neg |
| 589 | 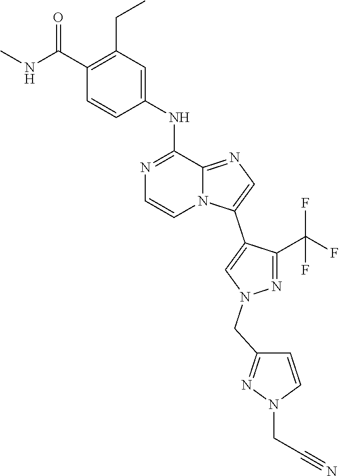 [SMILES: CCc1cc(Nc2nccn3c(-c4cn(Cc5ccn(CC#N)n5)nc4C(F)(F)F)cnc23)ccc1C(=O)NC] | 4-((3-(1-((1- (cyanomethyl)-1H- pyrazol-3-yl)methyl)-3- (trifluoromethyl)-1H- pyrazol-4- yl)imidazo[1,2-a] pyrazin-8- yl)amino)-2-ethyl-N- methylbenzamide | 548.5 | 549.3 |
| 590 | 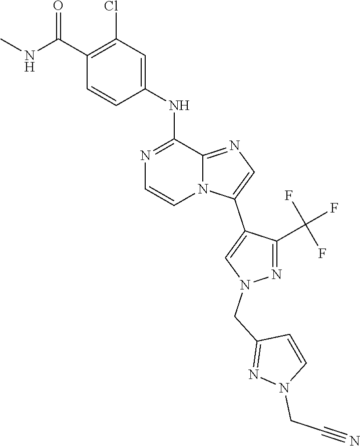 [SMILES: CNC(=O)c1ccc(Nc2nccn3c(-c4cn(Cc5ccn(CC#N)n5)nc4C(F)(F)F)cnc23)cc1Cl] | 2-chloro-4-((3-(1-((1- (cyanomethyl)-1H- pyrazol-3- yl)methyl)-3- (trifluoromethyl)-1H- pyrazol-4-yl)imidazo [1,2-a]pyrazin- 8-yl)amino)-N- methylbenzamide | 554.9 | 555.2 |
| 591 | 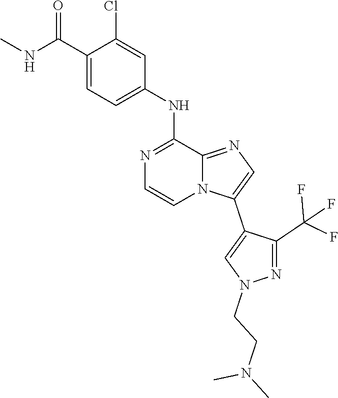 [SMILES: CNC(=O)c1ccc(Nc2nccn3c(-c4cn(CCN(C)C)nc4C(F)(F)F)cnc23)cc1Cl] | 2-chloro-4-((3-(1-(2- (dimethylamino)ethyl)-3- (trifluoromethyl)- 1H-pyrazol-4- yl)imidazo[1,2-a] pyrazin-8- yl)amino)-N- methylbenzamide formate | 506.9 | 507.2 |
| 592 | 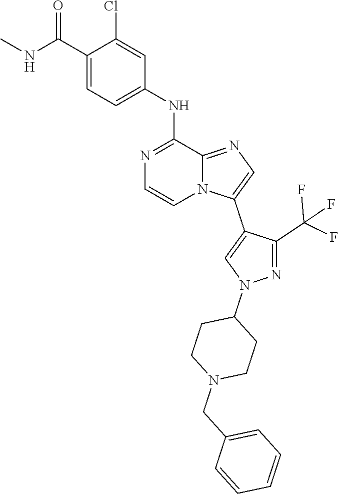 [SMILES: CNC(=O)c1ccc(Nc2nccn3c(-c4cn(C5CCN(Cc6ccccc6)CC5)nc4C(F)(F)F)cnc23)cc1Cl] | 4-((3-(1-(1- benzylpiperidin- 4-yl)-3- (trifluoromethyl)- 1H-pyrazol-4- yl)imidazo[1,2-a]pyrazin-8- yl)amino)-2-chloro-N- methylbenzamide formate | 609.1 | 609.3 |
| 593 | 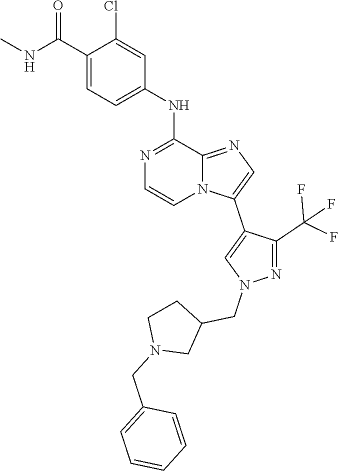 [SMILES: CNC(=O)c1ccc(Nc2nccn3c(-c4cn(CC5CCN(Cc6ccccc6)C5)nc4C(F)(F)F)cnc23)cc1Cl] | 4-((3-(1-((1- benzylpyrrolidin-3- yl)methyl)-3- (trifluoromethyl)-1H- pyrazol-4-yl)imidazo [1,2-a]pyrazin- 8-yl)amino)-2-chloro-N- methylbenzamide formate | 609.1 | 609.3 |
| 594 | 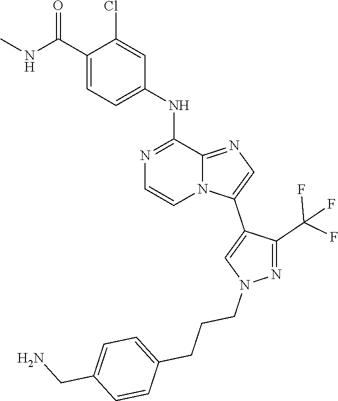 [SMILES: CNC(=O)c1ccc(Nc2nccn3c(-c4cn(CCCc5ccc(CN)cc5)nc4C(F)(F)F)cnc23)cc1Cl] | 4-((3-(1-(3-(4- (aminomethyl)phenyl) propyl)-3- (trifluoromethyl)- 1H-pyrazol-4- yl)imidazo[1,2-a] pyrazin-8- yl)amino)-2-chloro-N- methylbenzamide formate | 583.0 | 583.2 |
| 595 | 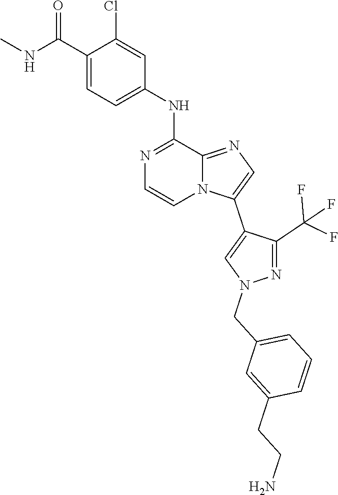 [SMILES: CNC(=O)c1ccc(Nc2nccn3c(-c4cn(Cc5cccc(CCN)c5)nc4C(F)(F)F)cnc23)cc1Cl] | 4-((3-(1-(3-(2- aminoethyl)benzyl)- 3-(trifluoromethyl)- 1H-pyrazol-4- yl)imidazo[1,2-a] pyrazin-8- yl)amino)-2-chloro-N- methylbenzamide formate | 569.0 | 569.3 |
| 598 | 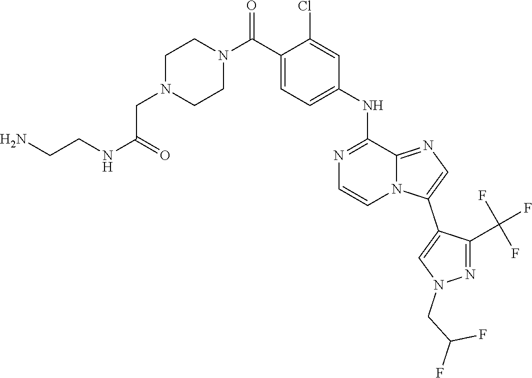 [SMILES: NCCNC(=O)CN1CCN(C(=O)c2ccc(Nc3nccn4c(-c5cn(CC(F)F)nc5C(F)(F)F)cnc34)cc2Cl)CC1] | N-(2-aminoethyl)-2- (4-(2-chloro-4- ((3-(1-(2,2- difluoroethyl)-3- (trifluoromethyl)- 1H-pyrazol-4- yl)imidazo[1,2-a] pyrazin-8- yl)amino)benzoyl) piperazin-1- yl)acetamide formate | 655.0 | 699.3 [M + HCOO]−, ESI neg |
| 599 | 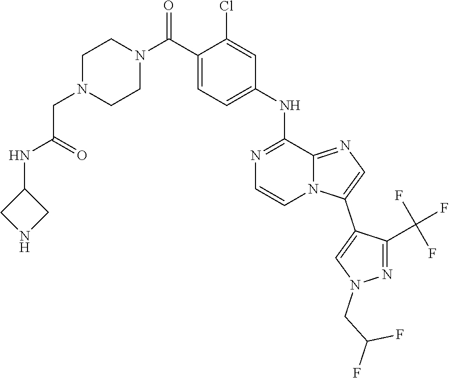 [SMILES: O=C(CN1CCN(C(=O)c2ccc(Nc3nccn4c(-c5cn(CC(F)F)nc5C(F)(F)F)cnc34)cc2Cl)CC1)NC1CNC1] | N-(azetidin-3-yl)-2- (4-(2-chloro-4- ((3-(1-(2,2- difluoroethyl)-3- (trifluoromethyl)- 1H-pyrazol-4- yl)imidazo[1,2-a] pyrazin-8- yl)amino)benzoyl) piperazin-1- yl)acetamide formate | 667.0 | 711.3 [M + HCOO]−, ESI neg |
| 600 | 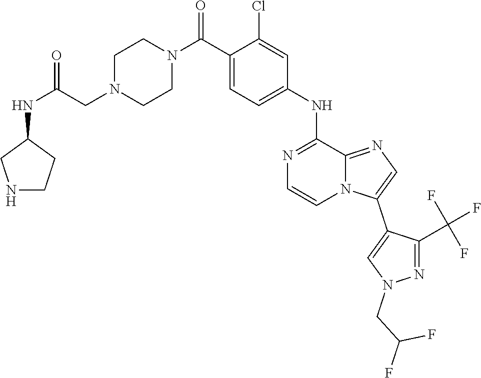 [SMILES: O=C(CN1CCN(C(=O)c2ccc(Nc3nccn4c(-c5cn(CC(F)F)nc5C(F)(F)F)cnc34)cc2Cl)CC1)N[C@H]1CCNC1] | (S)-2-(4-(2-chloro-4- ((3-(1-(2,2- difluoroethyl)-3- (trifluoromethyl)- 1H-pyrazol-4-yl) imidazo[1,2- a]pyrazin-8- yl)amino)benzoyl) piperazin-1-yl)- N-(pyrrolidin-3-yl) acetamide formate | 681.1 | 725.4 [M + HCOO]−, ESI neg |
| 601 | 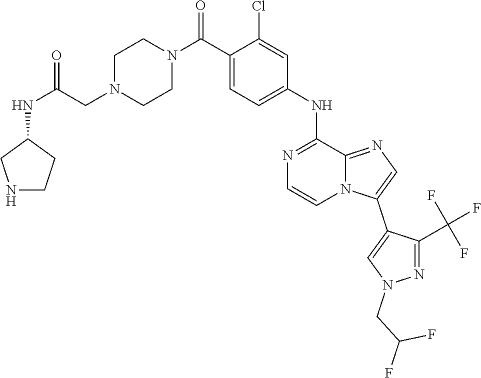 [SMILES: O=C(CN1CCN(C(=O)c2ccc(Nc3nccn4c(-c5cn(CC(F)F)nc5C(F)(F)F)cnc34)cc2Cl)CC1)N[C@@H]1CCNC1] | (R)-2-(4-(2-chloro-4- ((3-(1-(2,2- difluoroethyl)-3- (trifluoromethyl)- 1H-pyrazol-4-yl)imidazo [1,2-a]pyrazin-8- yl)amino)benzoyl) piperazin-1-yl)- N-(pyrrolidin-3-yl) acetamide formate | 681.1 | 725.3 [M + HCOO]−, ESI neg |
| 602 | 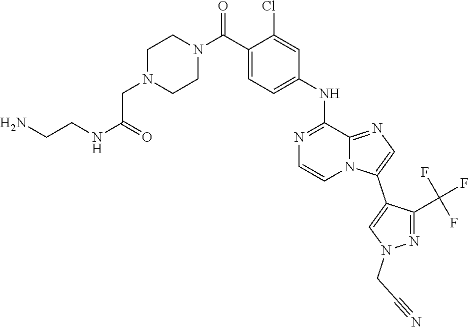 [SMILES: N#CCn1cc(-c2cnc3c(Nc4ccc(C(=O)N5CCN(CC(=O)NCCN)CC5)c(Cl)c4)nccn23)c(C(F)(F)F)n1] | N-(2-aminoethyl)-2- (4-(2-chloro-4- ((3-(1-(cyanomethyl)-3- (trifluoromethyl)-1H- pyrazol-4- yl)imidazo[1,2-a]pyrazin-8- yl)amino)benzoyl) piperazin-1- yl)acetamide formate | 630.0 | 674.3 [M + HCOO]−, ESI neg |
| 603 | 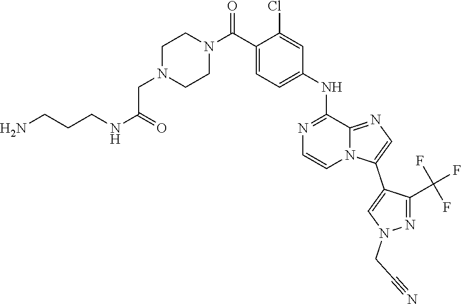 [SMILES: N#CCn1cc(-c2cnc3c(Nc4ccc(C(=O)N5CCN(CC(=O)NCCCN)CC5)c(Cl)c4)nccn23)c(C(F)(F)F)n1] | N-(3-aminopropyl)-2- (4-(2-chloro- 4-((3-(1-(cyanomethyl)-3- (trifluoromethyl)-1H- pyrazol-4- yl)imidazo[1,2-a]pyrazin-8- yl)amino)benzoyl) piperazin-1- yl)acetamide formate | 644.1 | 688.3 [M + HCOO]−, ESI neg |
| 604 | 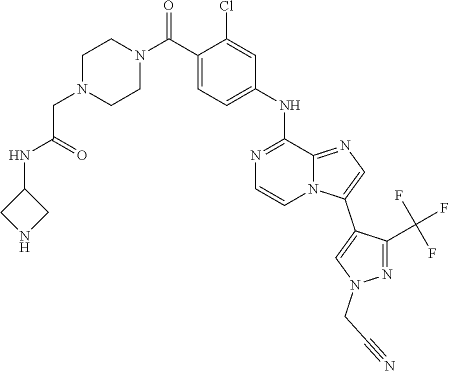 [SMILES: N#CCn1cc(-c2cnc3c(Nc4ccc(C(=O)N5CCN(CC(=O)NC6CNC6)CC5)c(Cl)c4)nccn23)c(C(F)(F)F)n1] | N-(azetidin-3-yl)-2- (4-(2-chloro-4- ((3-(1-(cyanomethyl)-3- (trifluoromethyl)-1H- pyrazol-4- yl)imidazo[1,2-a]pyrazin-8- yl)amino)benzoyl) piperazin-1- yl)acetamide formate | 642.0 | 686.3 [M + HCOO]−, ESI neg |
| 605 | 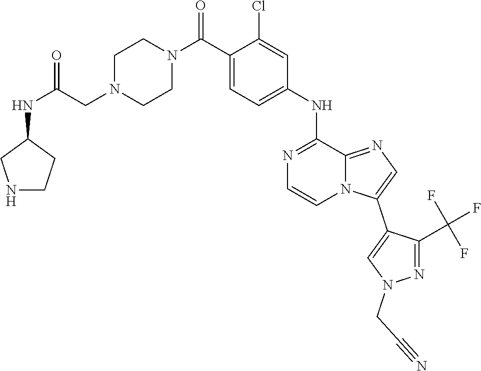 [SMILES: N#CCn1cc(-c2cnc3c(Nc4ccc(C(=O)N5CCN(CC(=O)N[C@H]6CCNC6)CC5)c(Cl)c4)nccn23)c(C(F)(F)F)n1] | (S)-2-(4-(2-chloro-4-((3-(1- (cyanomethyl)-3- (trifluoromethyl)- 1H-pyrazol-4-yl) imidazo[1,2- a]pyrazin-8- yl)amino)benzoyl) piperazin-1-yl)- N-(pyrrolidin-3-yl) acetamide formate | 656.1 | 700.3 [M + HCOO]−, ESI neg |
| 606 | 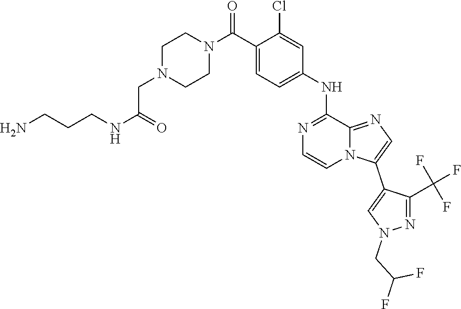 [SMILES: NCCCNC(=O)CN1CCN(C(=O)c2ccc(Nc3nccn4c(-c5cn(CC(F)F)nc5C(F)(F)F)cnc34)cc2Cl)CC1] | N-(3-aminopropyl)- 2-(4-(2-chloro- 4-((3-(1-(2,2- difluoroethyl)-3- (trifluoromethyl)- 1H-pyrazol-4- yl)imidazo[1,2-a] pyrazin-8- yl)amino)benzoyl) piperazin-1- yl)acetamide formate | 669.1 | 713.4 [M + HCOO]−, ESI neg |
| 607 | 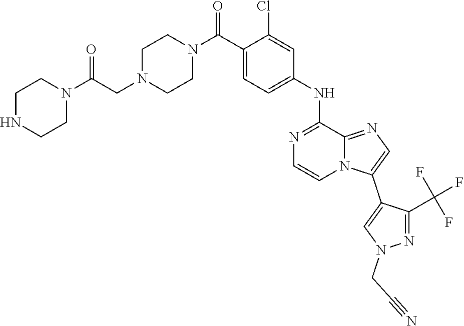 [SMILES: N#CCn1cc(-c2cnc3c(Nc4ccc(C(=O)N5CCN(CC(=O)N6CCNCC6)CC5)c(Cl)c4)nccn23)c(C(F)(F)F)n1] | 2-(4-(8-((3-chloro- 4-(4-(2-oxo-2- (piperazin-1-yl)ethyl) piperazine-1- carbonyl)phenyl) amino)imidazo[1,2- a]pyrazin-3-yl)-3- (trifluoromethyl)- 1H-pyrazol-1- yl)acetonitrile formate | 656.1 | 700.4 [M + HCOO]−, ESI neg |
| 608 | 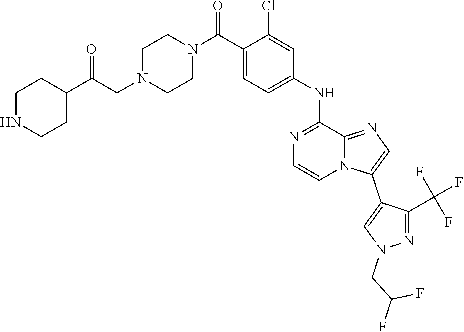 [SMILES: O=C(CN1CCN(C(=O)c2ccc(Nc3nccn4c(-c5cn(CC(F)F)nc5C(F)(F)F)cnc34)cc2Cl)CC1)C1CCNCC1] | 2-(4-(2-chloro-4- ((3-(1-(2,2- difluoroethyl)-3- (trifluoromethyl)- 1H-pyrazol-4-yl) imidazo[1,2- a]pyrazin-8- yl)amino)benzoyl) piperazin-1-yl)- 1-(piperazin-1-yl) ethan-1-one formate | 681.1 | 725.5 [M + HCOO]−, ESI neg |
| 609 | 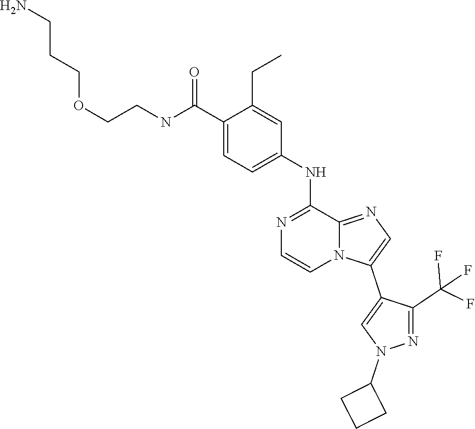 [SMILES: CCc1cc(Nc2nccn3c(-c4cn(C5CCC5)nc4C(F)(F)F)cnc23)ccc1C(=O)NCCOCCCN] | N-(2-(3-aminopropoxy) ethyl)-4-((3- (1-cyclobutyl-3- (trifluoromethyl)- 1H-pyrazol-4-yl) imidazo[1,2- a]pyrazin-8-yl)amino)-2- ethylbenzamide formate | 570.6 | 571.3 |
| 610 | 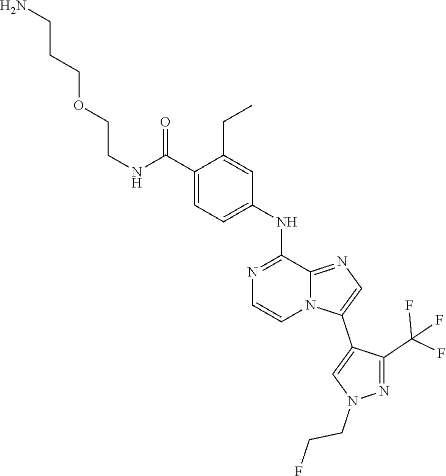 [SMILES: CCc1cc(Nc2nccn3c(-c4cn(CCF)nc4C(F)(F)F)cnc23)ccc1C(=O)NCCOCCCN] | N-(2-(3-aminopropoxy) ethyl)-2- ethyl-4-((3-(1-(2- fluoroethyl)-3- (trifluoromethyl)- 1H-pyrazol-4- yl)imidazo [1,2-a] pyrazin-8- yl)amino)benzamide formate | 562.6 | 563.3 |
| 611 | 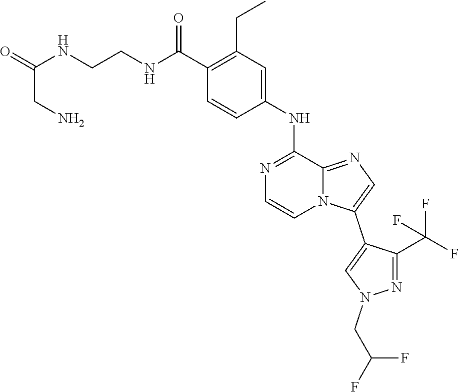 [SMILES: CCc1cc(Nc2nccn3c(-c4cn(CC(F)F)nc4C(F)(F)F)cnc23)ccc1C(=O)NCCNC(=O)CN] | N-(2-(2-aminoacetamido) ethyl)-4- ((3-(1-(2,2-difluoroethyl)-3- (trifluoromethyl)-1H- pyrazol-4- yl)imidazo[1,2-a]pyrazin-8- yl)amino)-2-ethylbenzamide formate | 579.5 | 580.3 |
| 612 | 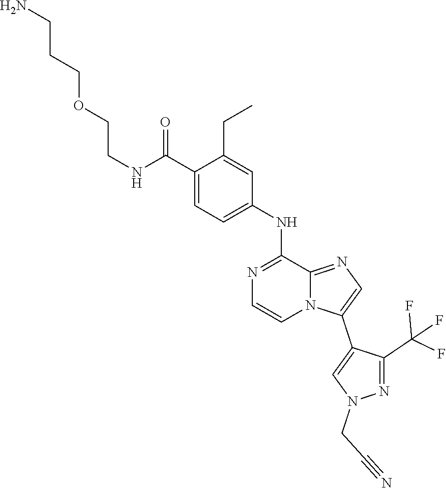 [SMILES: CCc1cc(Nc2nccn3c(-c4cn(CC#N)nc4C(F)(F)F)cnc23)ccc1C(=O)NCCOCCCN] | N-(2-(3-aminopropoxy) ethyl)-4-((3- (1-(cyanomethyl)-3- (trifluoromethyl)-1H- pyrazol-4- yl)imidazo[1,2-a] pyrazin-8- yl)amino)-2- ethylbenzamide formate | 555.6 | 556.3 |
| 613 | 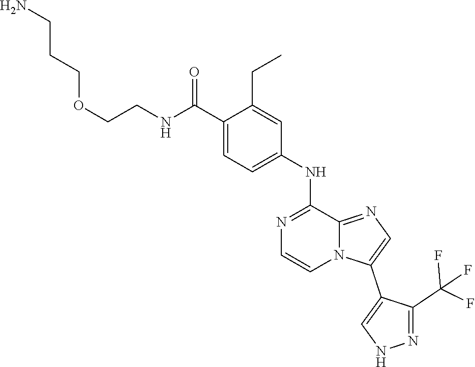 [SMILES: CCc1cc(Nc2nccn3c(-c4c[nH]nc4C(F)(F)F)cnc23)ccc1C(=O)NCCOCCCN] | N-(2-(3-aminopropoxy) ethyl)-2- ethyl-4-((3-(3- (trifluoromethyl)-1H- pyrazol-4-yl)imidazo [1,2-a]pyrazin- 8-yl)amino)benzamide formate | 516.5 | 517.3 |
| 614 | 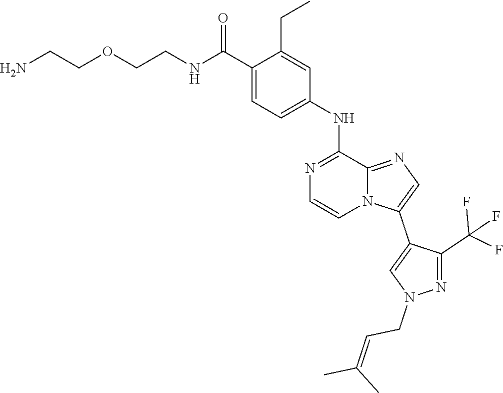 [SMILES: CCc1cc(Nc2nccn3c(-c4cn(CC=C(C)C)nc4C(F)(F)F)cnc23)ccc1C(=O)NCCOCCN] | N-(2-(2-aminoethoxy) ethyl)-2- ethyl-4-((3-(1-(3- methylbut-2-en-1- yl)-3-(trifluoromethyl)- 1H-pyrazol- 4-yl)imidazo[1,2-a] pyrazin-8- yl)amino)benzamide formate | 570.6 | 571.3 |
| 615 | 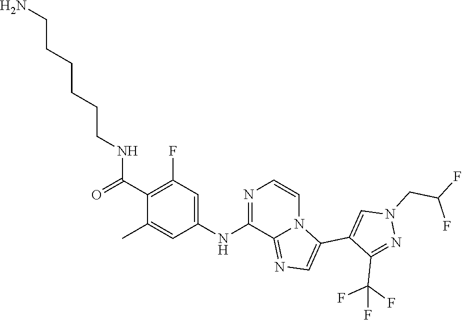 [SMILES: Cc1cc(Nc2nccn3c(-c4cn(CC(F)F)nc4C(F)(F)F)cnc23)cc(F)c1C(=O)NCCCCCCN] | N-(6-aminohexyl)-4- ((3-(1-(2,2- difluoroethyl)-3- (trifluoromethyl)- 1H-pyrazol-4-yl) imidazo[1,2- a]pyrazin-8-yl)amino)-2- fluoro-6- methylbenzamide | 582.6 | 583.3 |
| 616 | 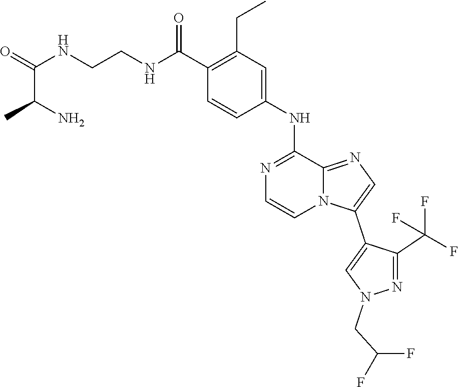 [SMILES: CCc1cc(Nc2nccn3c(-c4cn(CC(F)F)nc4C(F)(F)F)cnc23)ccc1C(=O)NCCNC(=O)[C@H](C)N] | (S)-N-(2-(2- aminopropanamido) ethyl)-4-((3-(1- (2,2-difluoroethyl)-3- (trifluoromethyl)- 1H-pyrazol-4- yl)imidazo[1,2-a] pyrazin-8- yl)amino)-2- ethylbenzamide formate | 593.6 | 594.3 |
| 617 | 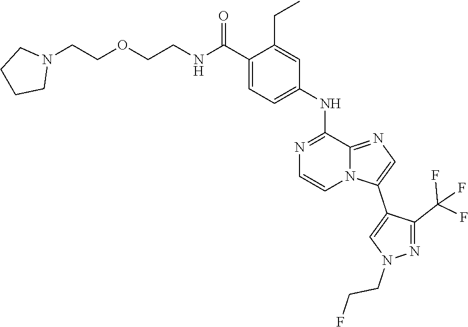 [SMILES: CCc1cc(Nc2nccn3c(-c4cn(CCF)nc4C(F)(F)F)cnc23)ccc1C(=O)NCCOCCN1CCCC1] | 2-ethyl-4-((3-(1-(2- fluoroethyl)-3- (trifluoromethyl)- 1H-pyrazol-4- yl)imidazo[1,2-a] pyrazin-8- yl)amino)-N-(2-(2- (pyrrolidin-1- yl)ethoxy)ethyl) benzamide formate | 602.6 | 603.4 |
| 618 | 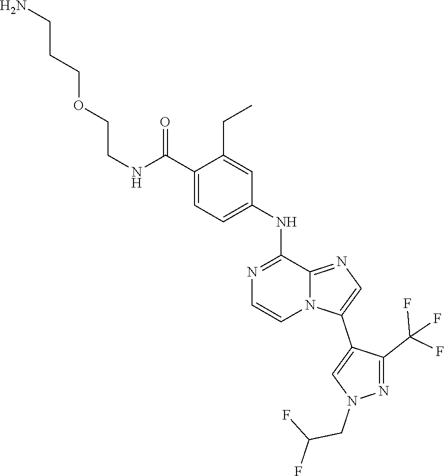 [SMILES: CCc1cc(Nc2nccn3c(-c4cn(CC(F)F)nc4C(F)(F)F)cnc23)ccc1C(=O)NCCOCCCN] | N-(2-(3-aminopropoxy) ethyl)-4-((3- (1-(2,2-difluoroethyl)-3- (trifluoromethyl)-1H- pyrazol-4- yl)imidazo[1,2-a] pyrazin-8- yl)amino)-2- ethylbenzamide formate | 580.6 | 581.3 |
| 619 | 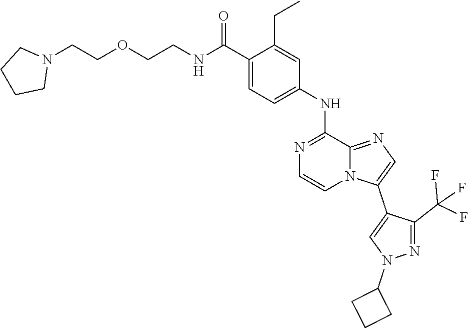 [SMILES: CCc1cc(Nc2nccn3c(-c4cn(C5CCC5)nc4C(F)(F)F)cnc23)ccc1C(=O)NCCOCCN1CCCC1] | 4-((3-(1-cyclobutyl-3- (trifluoromethyl)-1H- pyrazol-4- yl)imidazo[1,2-a] pyrazin-8- yl)amino)-2-ethyl-N-(2-(2- (pyrrolidin-1- yl)ethoxy)ethyl)benzamide formate | 610.7 | 611.4 |
| 620 | 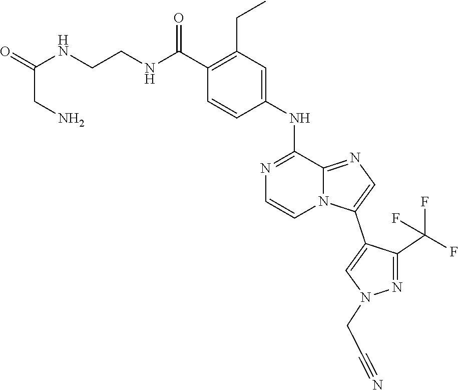 [SMILES: CCc1cc(Nc2nccn3c(-c4cn(CC#N)nc4C(F)(F)F)cnc23)ccc1C(=O)NCCNC(=O)CN] | N-(2-(2-aminoacetamido) ethyl)-4- ((3-(1-(cyanomethyl)-3- (trifluoromethyl)- 1H-pyrazol-4- yl)imidazo[1,2-a]pyrazin-8- yl)amino)-2-ethylbenzamide formate | 554.5 | 555.3 |
| 621 | 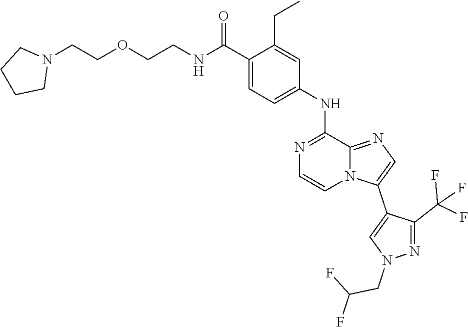 [SMILES: CCc1cc(Nc2nccn3c(-c4cn(CC(F)F)nc4C(F)(F)F)cnc23)ccc1C(=O)NCCOCCN1CCCC1] | 4-((3-(1-(2,2- difluoroethyl)-3- (trifluoromethyl)- 1H-pyrazol-4- yl)imidazo[1,2-a]pyrazin- 8-yl)amino)-2-ethyl-N- (2-(2-(pyrrolidin-1- yl)ethoxy)ethyl) benzamide formate | 620.6 | 621.4 |
| 622 | 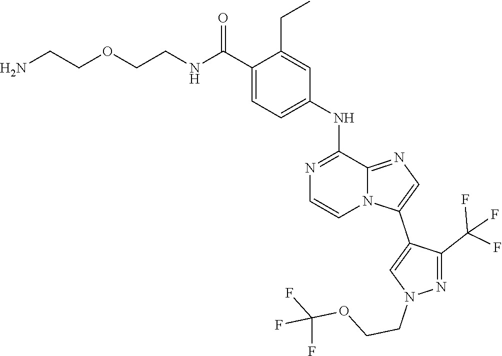 [SMILES: CCc1cc(Nc2nccn3c(-c4cn(CCOC(F)(F)F)nc4C(F)(F)F)cnc23)ccc1C(=O)NCCOCCN] | N-(2-(2-aminoethoxy) ethyl)-2- ethyl-4-((3-(1-(2- (trifluoromethoxy)ethyl)-3- (trifluoromethyl)- 1H-pyrazol-4- yl)imidazo[1,2-a] pyrazin-8- yl)amino)benzamide | 614.5 | 615.4 |
| 623 | 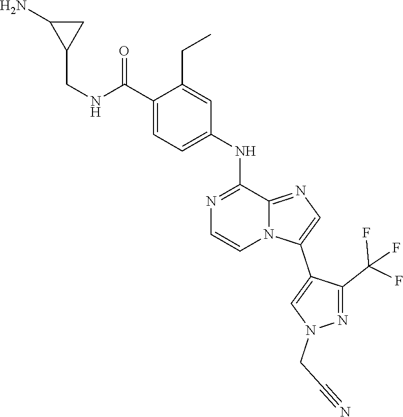 [SMILES: CCc1cc(Nc2nccn3c(-c4cn(CC#N)nc4C(F)(F)F)cnc23)ccc1C(=O)NCC1CC1N] | N-((2-aminocyclopropyl) methyl)-4- ((3-(1-(cyanomethyl)-3- (trifluoromethyl)- 1H-pyrazol-4- yl)imidazo[1,2-a]pyrazin-8- yl)amino)-2-ethylbenzamide formate | 523.5 | 524.3 |
| 624 | 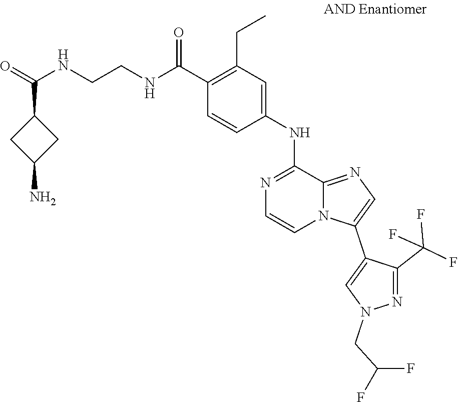 [SMILES: CCc1cc(Nc2nccn3c(-c4cn(CC(F)F)nc4C(F)(F)F)cnc23)ccc1C(=O)NCCNC(=O)[C@H]1C[C@@H](N)C1] | cis-N-(2-((1s,3s)-3- aminocyclobutane-1- carboxamido)ethyl)- 4-((3-(1-(2,2- difluoroethyl)-3- (trifluoromethyl)- 1H-pyrazol-4- yl)imidazo[1,2- a]pyrazin-8-yl)amino)-2- ethylbenzamide formate | 619.6 | 620.3 |
| 625 | 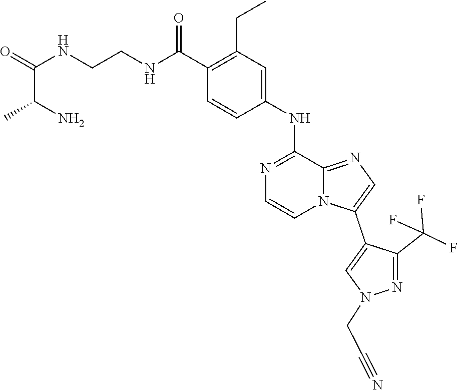 [SMILES: CCc1cc(Nc2nccn3c(-c4cn(CC#N)nc4C(F)(F)F)cnc23)ccc1C(=O)NCCNC(=O)[C@@H](C)N] | (R)-N-(2-(2- aminopropanamido) ethyl)-4-((3-(1- (cyanomethyl)-3- (trifluoromethyl)- 1H-pyrazol-4-yl) imidazo[1,2- a]pyrazin-8-yl)amino)-2- ethylbenzamide formate | 568.6 | 569.3 |
| 626 | 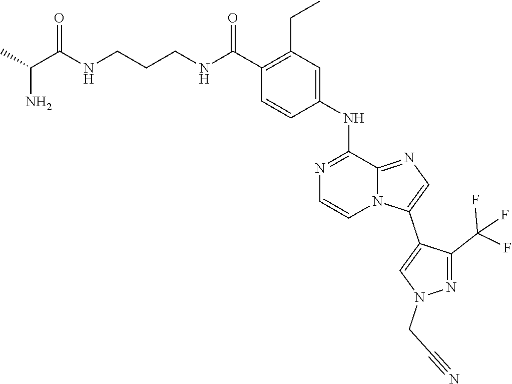 [SMILES: CCc1cc(Nc2nccn3c(-c4cn(CC#N)nc4C(F)(F)F)cnc23)ccc1C(=O)NCCCNC(=O)[C@@H](C)N] | (R)-N-(3-(2- aminopropanamido) propyl)-4-((3- (1-(cyanomethyl)-3- (trifluoromethyl)- 1H-pyrazol-4- yl)imidazo[1,2-a]pyrazin-8- yl)amino)-2-ethylbenzamide formate | 582.6 | 627.5 [M − HCOO]−, ESI neg |
| 627 | 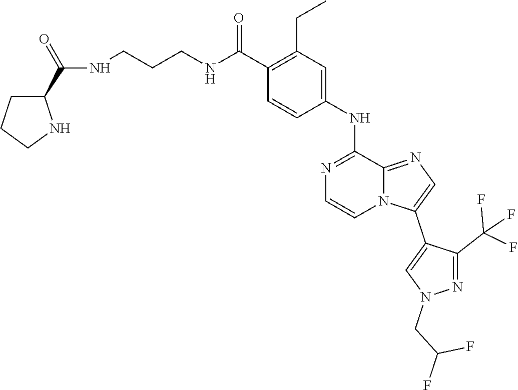 [SMILES: CCc1cc(Nc2nccn3c(-c4cn(CC(F)F)nc4C(F)(F)F)cnc23)ccc1C(=O)NCCCNC(=O)[C@@H]1CCCN1] | (S)-N-(3-(4-((3-(1-(2,2- difluoroethyl)-3- (trifluoromethyl)- 1H-pyrazol-4-yl) imidazo[1,2- a]pyrazin-8-yl)amino)-2- ethylbenzamido)propyl) pyrrolidine- 2-carboxamide | 633.6 | 632.5 [M − H]−, ESI neg |
| 628 | 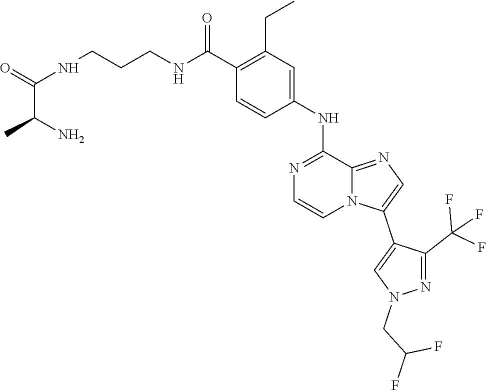 [SMILES: CCc1cc(Nc2nccn3c(-c4cn(CC(F)F)nc4C(F)(F)F)cnc23)ccc1C(=O)NCCCNC(=O)[C@H](C)N] | (S)-N-(3-(2- aminopropanamido) propyl)-4-((3- (1-(2,2-difluoroethyl)-3- (trifluoromethyl)- 1H-pyrazol-4- yl)imidazo[1,2-a] pyrazin-8- yl)amino)-2- ethylbenzamide formate | 607.6 | 652.5 [M − HCOO]−, ESI neg |
| 629 | 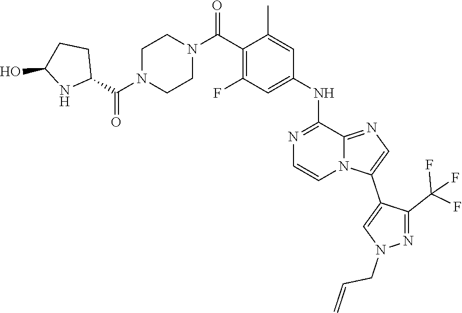 [SMILES: C=CCn1cc(-c2cnc3c(Nc4cc(C)c(C(=O)N5CCN(C(=O)[C@H]6CC[C@H](O)N6)CC5)c(F)c4)nccn23)c(C(F)(F)F)n1] | (4-(4-((3-(1-allyl-3- (trifluoromethyl)- 1H-pyrazol-4- yl)imidazo[1,2-a] pyrazin-8- yl)amino)-2-fluoro-6- methylbenzoyl)piperazin-1- yl)((2R,5S)-5- hydroxypyrrolidin-2- yl)methanone | 641.6 | 642.4 |
| 630 | 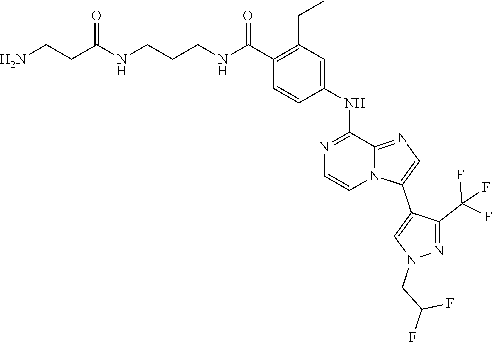 [SMILES: CCc1cc(Nc2nccn3c(-c4cn(CC(F)F)nc4C(F)(F)F)cnc23)ccc1C(=O)NCCCNC(=O)CCN] | N-(3-(3- aminopropanamido) propyl)-4-((3- (1-(2,2-difluoroethyl)-3- (trifluoromethyl)- 1H-pyrazol-4- yl)imidazo[1,2-a]pyrazin-8- yl)amino)-2- ethylbenzamide formate | 607.6 | 652.5 [M − HCOO]−, ESI neg |
| 631 | 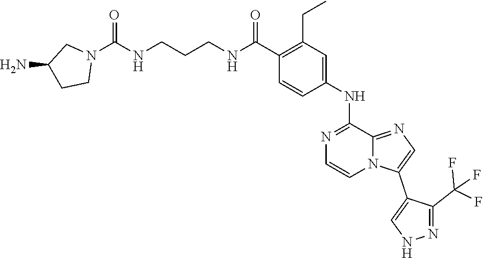 [SMILES: CCc1cc(Nc2nccn3c(-c4c[nH]nc4C(F)(F)F)cnc23)ccc1C(=O)NCCCNC(=O)N1CC[C@@H](N)C1] | (R)-3-amino-N-(3-(2- ethyl-4-((3-(3- (trifluoromethyl)- 1H-pyrazol-4- yl)imidazo[1,2-a]pyrazin-8- yl)amino)benzamido)propyl) pyrrolidine-1-carboxamide | 584.6 | 583.4 [M − H]−, ESI neg |
| 632 | 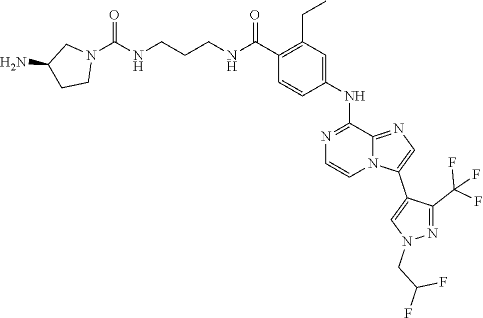 [SMILES: CCc1cc(Nc2nccn3c(-c4cn(CC(F)F)nc4C(F)(F)F)cnc23)ccc1C(=O)NCCCNC(=O)N1CC[C@@H](N)C1] | (R)-3-amino-N-(3- (4-((3-(1-(2,2- difluoroethyl)-3- (trifluoromethyl)- 1H-pyrazol-4-yl) imidazo[1,2- a]pyrazin-8-yl)amino)-2- ethylbenzamido)propyl) pyrrolidine- 1-carboxamide formate | 648.6 | 693.6 [M + HCOO]−, ESI neg. |
| 633 | 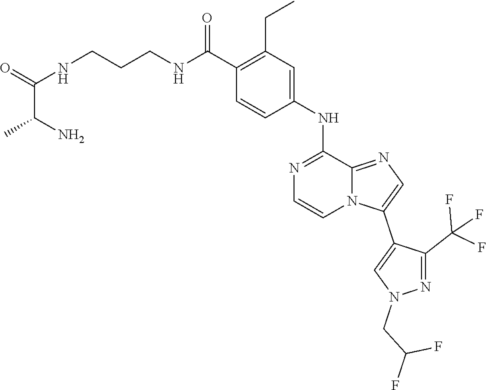 [SMILES: CCc1cc(Nc2nccn3c(-c4cn(CC(F)F)nc4C(F)(F)F)cnc23)ccc1C(=O)NCCCNC(=O)[C@@H](C)N] | (R)-N-(3-(2- aminopropanamido) propyl)-4-((3- (1-(2,2-difluoroethyl)-3- (trifluoromethyl)- 1H-pyrazol-4- yl)imidazo[1,2-a] pyrazin-8- yl)amino)-2- ethylbenzamide formate | 607.6 | 652.5 [M + HCOO]−, ESI neg. |
| 634 | 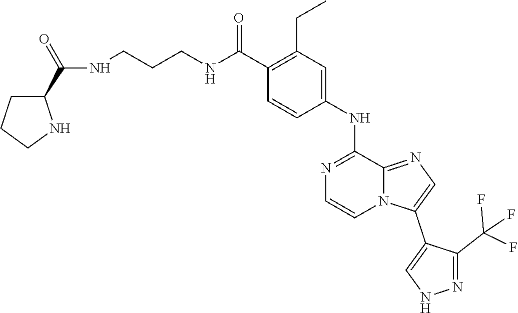 [SMILES: CCc1cc(Nc2nccn3c(-c4c[nH]nc4C(F)(F)F)cnc23)ccc1C(=O)NCCCNC(=O)[C@@H]1CCCN1] | (S)-N-(3-(2-ethyl-4-((3-(3- (trifluoromethyl)- 1H-pyrazol-4- yl)imidazo[1,2-a] pyrazin-8- yl)amino) benzamido)propyl) pyrrolidine-2- carboxamide | 569.6 | 568.4 [M − H]−, ESI neg |
| 635 | 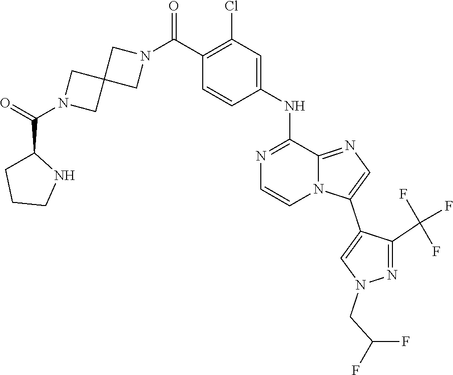 [SMILES: O=C(c1ccc(Nc2nccn3c(-c4cn(CC(F)F)nc4C(F)(F)F)cnc23)cc1Cl)N1CC2(C1)CN(C(=O)[C@@H]1CCCN1)C2] | (S)-(2-chloro-4-((3-(1- (2,2-difluoroethyl)-3- (trifluoromethyl)- 1H-pyrazol-4-yl) imidazo[1,2- a]pyrazin-8-yl)amino) phenyl)(6- prolyl-2,6-diazaspiro [3.3]heptan-2- yl)methanone | 664.0 | 662.5 [M − H]−, ESI neg |
| 636 | 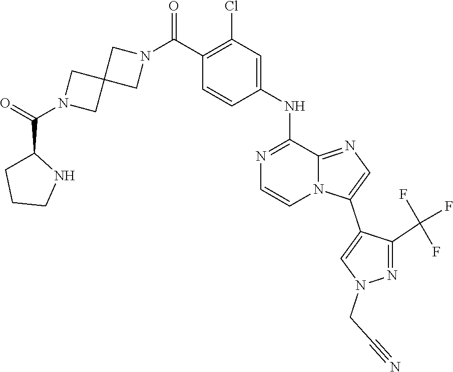 [SMILES: N#CCn1cc(-c2cnc3c(Nc4ccc(C(=O)N5CC6(C5)CN(C(=O)[C@@H]5CCCN5)C6)c(Cl)c4)nccn23)c(C(F)(F)F)n1] | (S)-2-(4-(8-((3-chloro- 4-(6-prolyl- 2,6-diazaspiro[3.3]heptane-2- carbonyl)phenyl)amino) imidazo[1,2- a]pyrazin-3-yl)-3- (trifluoromethyl)-1H- pyrazol-1- yl)acetonitrile | 639.0 | 637.6 [M − H]−, ESI neg |
| 637 | 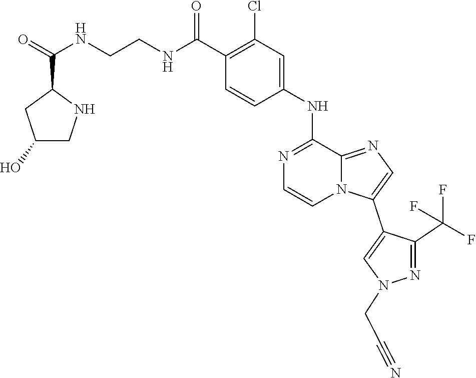 [SMILES: N#CCn1cc(-c2cnc3c(Nc4ccc(C(=O)NCCNC(=O)[C@@H]5C[C@@H](O)CN5)c(Cl)c4)nccn23)c(C(F)(F)F)n1] | (2S,4R)-N-(2-(4-((3-(1- (cyanomethyl)-3- (trifluoromethyl)- 1H-pyrazol-4-yl)imidazo[1,2- a]pyrazin-8-yl)amino)-2- ethylbenzamido)ethyl)-4- hydroxypyrrolidine-2- carboxamide | 610.6 | 611.4 |
| 638 | 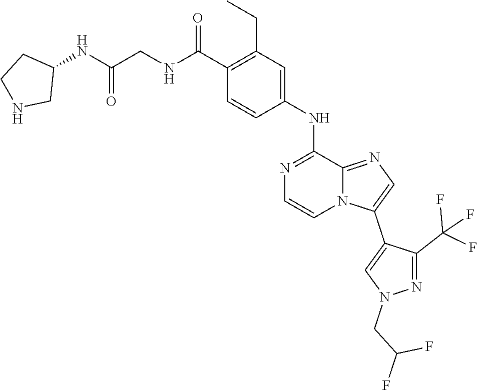 [SMILES: CCc1cc(Nc2nccn3c(-c4cn(CC(F)F)nc4C(F)(F)F)cnc23)ccc1C(=O)NCC(=O)N[C@H]1CCNC1] | 4-[[3-[1-(2,2- difluoroethyl)-3- (trifluoromethyl)pyrazol- 4-yl]imidazo[1,2-a] pyrazin-8- yl]amino]-2-ethyl-N- [2-oxo-2- [[(3S)-pyrrolidin-3- yl]amino]ethyl] benzamide formate | 605.6 | 606.2 |
| 639 | 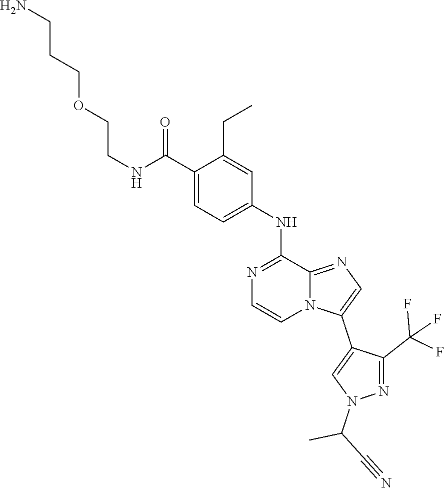 [SMILES: CCc1cc(Nc2nccn3c(-c4cn(C(C)C#N)nc4C(F)(F)F)cnc23)ccc1C(=O)NCCOCCCN] | N-(2-(3-aminopropoxy) ethyl)-4-((3- (1-(1-cyanoethyl)-3- (trifluoromethyl)- 1H-pyrazol-4- yl)imidazo[1,2-a] pyrazin-8- yl)amino)-2- ethylbenzamide formate | 569.6 | 570.3 |
| 640 | 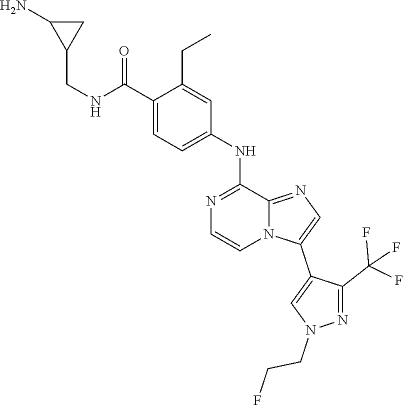 [SMILES: CCc1cc(Nc2nccn3c(-c4cn(CCF)nc4C(F)(F)F)cnc23)ccc1C(=O)NCC1CC1N] | N-((2-aminocyclopropyl) methyl)-2- ethyl-4-((3-(1-(2- fluoroethyl)-3- (trifluoromethyl)-1H- pyrazol-4- yl)imidazo[1,2-a] pyrazin-8- yl)amino) benzamide formate | 530.5 | 531.3 |
| 641 | 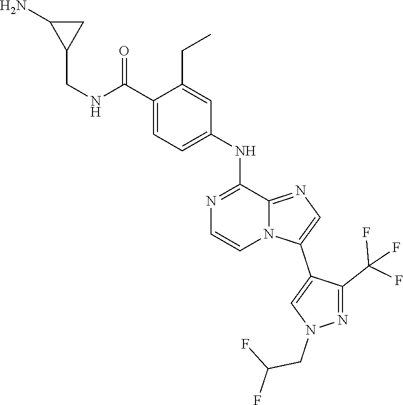 [SMILES: CCc1cc(Nc2nccn3c(-c4cn(CC(F)F)nc4C(F)(F)F)cnc23)ccc1C(=O)NCC1CC1N] | N-((2-aminocyclopropyl) methyl)-4- ((3-(1-(2,2-difluoroethyl)-3- (trifluoromethyl)- 1H-pyrazol-4- yl)imidazo[1,2-a]pyrazin- 8-yl)amino)-2- ethylbenzamide formate | 548.5 | 549.3 |
| 642 | 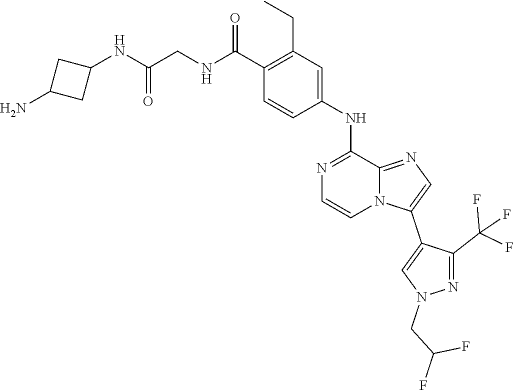 [SMILES: CCc1cc(Nc2nccn3c(-c4cn(CC(F)F)nc4C(F)(F)F)cnc23)ccc1C(=O)NCC(=O)NC1CC(N)C1] | N-[2-[(3-aminocyclobutyl) amino]- 2-oxo-ethyl]-4-[[3-[1-(2,2- difluoroethyl)-3- (trifluoromethyl)pyrazol-4- yl]imidazo[1,2-a]pyrazin-8- yl]amino]-2-ethyl- benzamide formate | 605.6 | 606.1 |
| 643 | 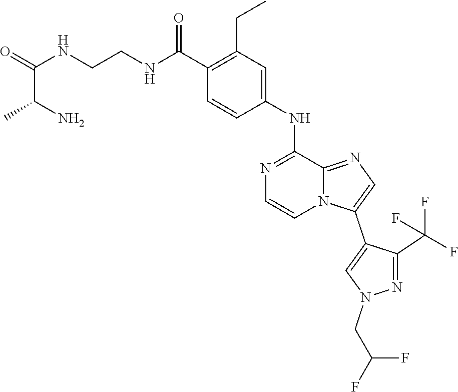 [SMILES: CCc1cc(Nc2nccn3c(-c4cn(CC(F)F)nc4C(F)(F)F)cnc23)ccc1C(=O)NCCNC(=O)[C@@H](C)N] | (R)-N-(2-(2- aminopropanamido) ethyl)-4-((3-(1- (2,2-difluoroethyl)-3- (trifluoromethyl)- 1H-pyrazol-4- yl)imidazo[1,2-a]pyrazin-8- yl)amino)-2-ethylbenzamide formate | 593.6 | 594.3 |
| 644 | 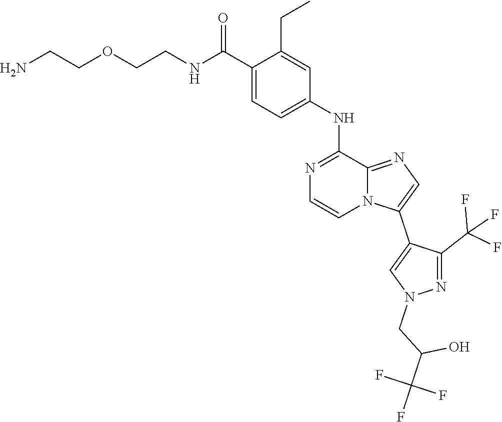 [SMILES: CCc1cc(Nc2nccn3c(-c4cn(CC(O)C(F)(F)F)nc4C(F)(F)F)cnc23)ccc1C(=O)NCCOCCN] | N-(2-(2- aminoethoxy)ethyl)-2- ethyl-4-((3-(1- (3,3,3-trifluoro-2- hydroxypropyl)-3- (trifluoromethyl)- 1H-pyrazol-4-yl)imidazo [1,2-a]pyrazin-8-yl)amino) benzamide | 614.5 | 615.3 |
| 645 | 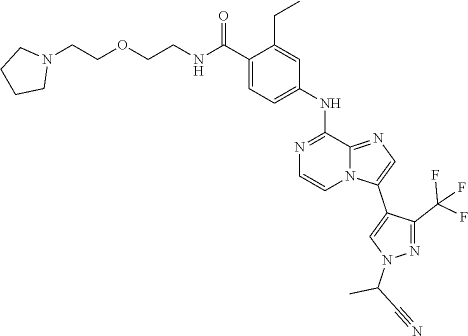 [SMILES: CCc1cc(Nc2nccn3c(-c4cn(C(C)C#N)nc4C(F)(F)F)cnc23)ccc1C(=O)NCCOCCN1CCCC1] | 4-((3-(1-(1-cyanoethyl)-3- (trifluoromethyl)-1H- pyrazol-4- yl)imidazo[1,2-a]pyrazin-8- yl)amino)-2-ethyl-N-(2-(2- (pyrrolidin-1- yl)ethoxy)ethyl)benzamide formate | 609.7 | 610.4 |
| 646 | 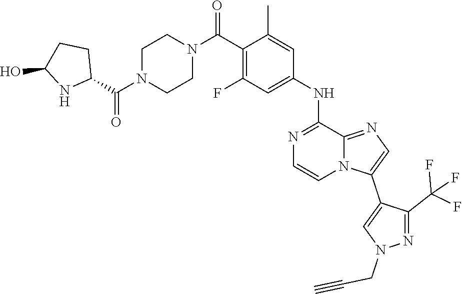 [SMILES: C#CCn1cc(-c2cnc3c(Nc4cc(C)c(C(=O)N5CCN(C(=O)[C@H]6CC[C@H](O)N6)CC5)c(F)c4)nccn23)c(C(F)(F)F)n1] | (4-(2-fluoro-6-methyl- 4-((3-(1- (prop-2-yn-1-yl)-3- (trifluoromethyl)- 1H-pyrazol-4- yl)imidazo[1,2-a]pyrazin- 8-yl)amino)benzoyl) piperazin-1- yl)((2R,5S)-5- hydroxypyrrolidin-2- yl)methanone | 639.6 | 640.4 |
| 647 | 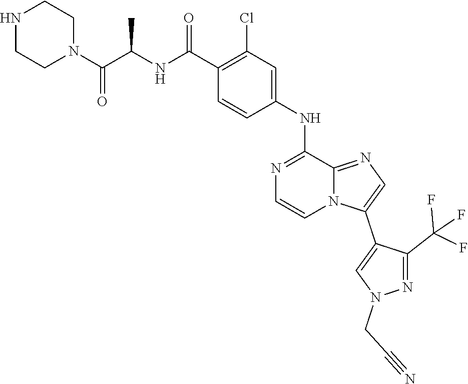 [SMILES: C[C@@H](NC(=O)c1ccc(Nc2nccn3c(-c4cn(CC#N)nc4C(F)(F)F)cnc23)cc1Cl)C(=O)N1CCNCC1] | 2-chloro-4-[[3-[1- (cyanomethyl)-3- (trifluoromethyl)pyrazol-4- yl]imidazo[1,2-a]pyrazin-8- yl]amino]-N-[(1R)-1- methyl-2-oxo- 2-piperazin-1-yl- ethyl]benzamide formate | 601.0 | 601.1 |
| 648 | 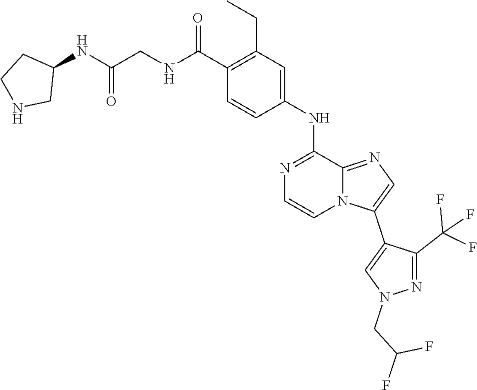 [SMILES: CCc1cc(Nc2nccn3c(-c4cn(CC(F)F)nc4C(F)(F)F)cnc23)ccc1C(=O)NCC(=O)N[C@@H]1CCNC1] | 4-[[3-[1-(2,2- difluoroethyl)-3- (trifluoromethyl)pyrazol-4- yl]imidazo[1,2-a]pyrazin-8- yl]amino]-2-ethyl- N-[2-oxo-2- [[(3R)-pyrrolidin-3- yl]amino]ethyl] benzamide formate | 605.6 | 606.1 |
| 649 | 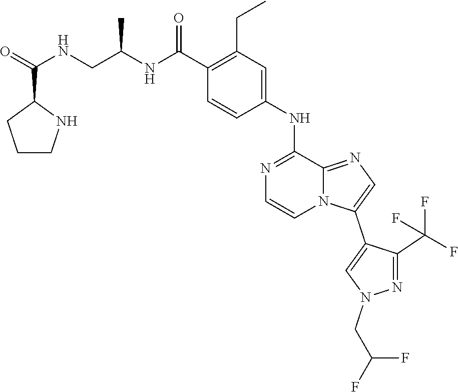 [SMILES: CCc1cc(Nc2nccn3c(-c4cn(CC(F)F)nc4C(F)(F)F)cnc23)ccc1C(=O)N[C@H](C)CNC(=O)[C@@H]1CCCN1] | (S)-N-((R)-2-(4-((3-(1- (2,2-difluoroethyl)-3- (trifluoromethyl)- 1H-pyrazol-4-yl)i midazo[1,2- a]pyrazin-8-yl)amino)-2- ethylbenzamido) propyl)pyrrolidine- 2-carboxamide | 633.6 | 634.4 |
| 650 | 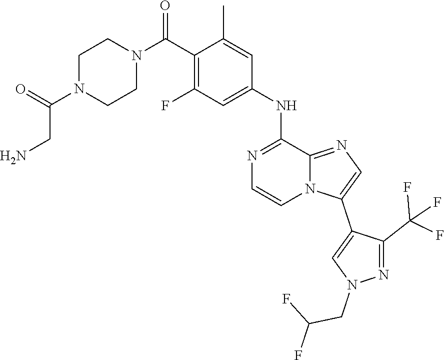 [SMILES: Cc1cc(Nc2nccn3c(-c4cn(CC(F)F)nc4C(F)(F)F)cnc23)cc(F)c1C(=O)N1CCN(C(=O)CN)CC1] | 2-amino-1-(4-(4-((3-(1- (2,2-difluoroethyl)-3- (trifluoromethyl)- 1H-pyrazol-4-yl) imidazo[1,2- a]pyrazin-8-yl)amino)- 2-fluoro-6- methylbenzoyl)piperazin- 1-yl)ethan-1-one | 609.5 | 610.4 |
| 651 | 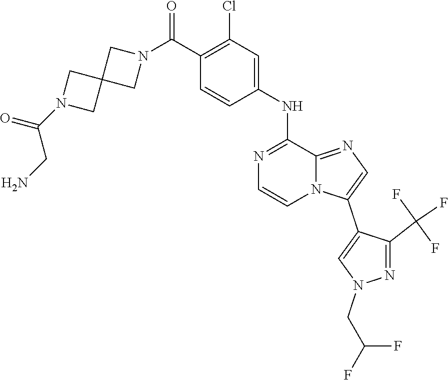 [SMILES: NCC(=O)N1CC2(C1)CN(C(=O)c1ccc(Nc3nccn4c(-c5cn(CC(F)F)nc5C(F)(F)F)cnc34)cc1Cl)C2] | 2-amino-1-(6-(2-chloro- 4-((3-(1- (2,2-difluoroethyl)-3- (trifluoromethyl)- 1H-pyrazol-4- yl)imidazo[1,2-a]pyrazin- 8-yl)amino)benzoyl)-2,6- diazaspiro[3.3]heptan- 2-yl)ethan-1- one | 624.0 | 622.4 [M − H]−, ESI neg |
| 652 | 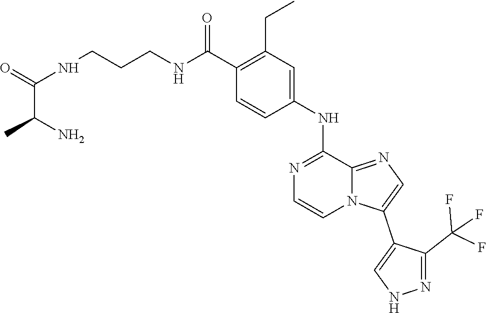 [SMILES: CCc1cc(Nc2nccn3c(-c4c[nH]nc4C(F)(F)F)cnc23)ccc1C(=O)NCCCNC(=O)[C@H](C)N] | (S)-N-(3-(2- aminopropanamido) propyl)-2-ethyl- 4-((3-(3-(trifluoromethyl)- 1H-pyrazol-4-yl)imidazo [1,2-a]pyrazin- 8-yl)amino)benzamide formate | 543.6 | 542.4 [M − H]−, ESI neg |
| 653 | 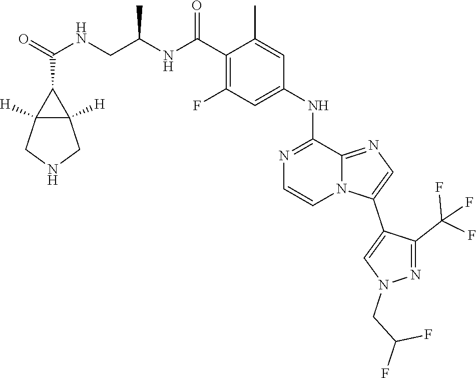 [SMILES: Cc1cc(Nc2nccn3c(-c4cn(CC(F)F)nc4C(F)(F)F)cnc23)cc(F)c1C(=O)N[C@H](C)CNC(=O)[C@H]1[C@@H]2CNC[C@@H]21] | (1R,5S,6r)-N-((R)-2- (4-((3-(1-(2,2- difluoroethyl)-3- (trifluoromethyl)- 1H-pyrazol-4-yl)imidazo [1,2- a]pyrazin-8-yl)amino)-2- fluoro-6- methylbenzamido) propyl)-3- azabicyclo[3.1.0]hexane- 6-carboxamide | 649.6 | 650.4 |
| 654 | 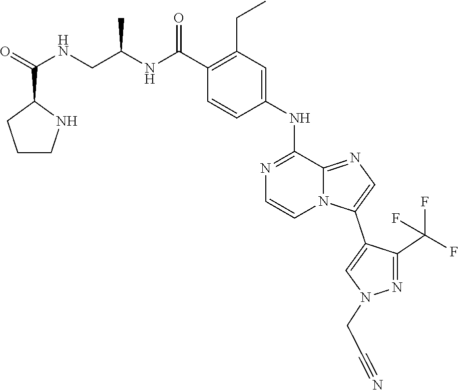 [SMILES: CCc1cc(Nc2nccn3c(-c4cn(CC#N)nc4C(F)(F)F)cnc23)ccc1C(=O)N[C@H](C)CNC(=O)[C@@H]1CCCN1] | (S)-N-((R)-2-(4-((3-(1- (cyanomethyl)-3- (trifluoromethyl)- 1H-pyrazol-4-yl)imidazo[1,2- a]pyrazin-8-yl)amino)-2- ethylbenzamido)propyl) pyrrolidine- 2-carboxamide | 608.6 | 609.4 |
| 655 | 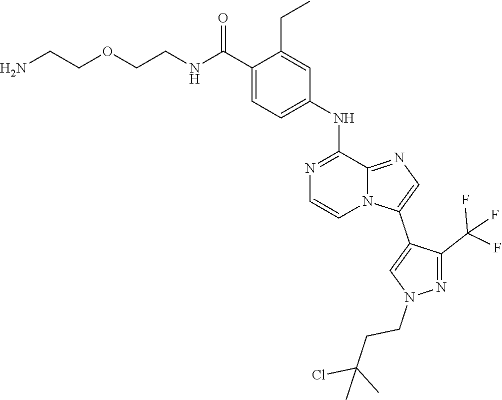 [SMILES: CCc1cc(Nc2nccn3c(-c4cn(CCC(C)(C)Cl)nc4C(F)(F)F)cnc23)ccc1C(=O)NCCOCCN] | N-(2-(2-aminoethoxy) ethyl)-4-((3- (1-(3-chloro-3- methylbutyl)-3- (trifluoromethyl)- 1H-pyrazol-4- yl)imidazo[1,2-a] pyrazin-8- yl)amino)-2- ethylbenzamide | 607.1 | 607.2 |
| 656 | 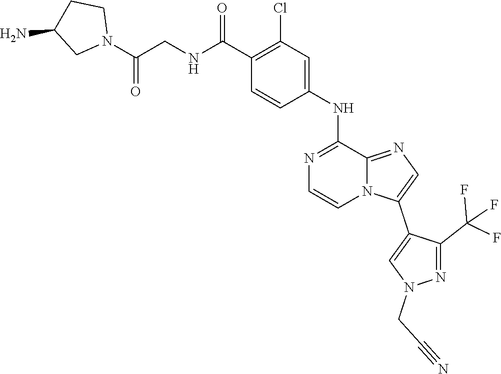 [SMILES: N#CCn1cc(-c2cnc3c(Nc4ccc(C(=O)NCC(=O)N5CC[C@H](N)C5)c(Cl)c4)nccn23)c(C(F)(F)F)n1] | N-[2-[(3S)-3- aminopyrrolidin-1-yl]- 2-oxo-ethyl]-2- chloro-4-[[3-[1- (cyanomethyl)-3- (trifluoromethyl)pyrazol-4- yl]imidazo[1,2-a]pyrazin- 8-yl]amino] benzamide formate | 587.0 | 587.1 |
| 658 | 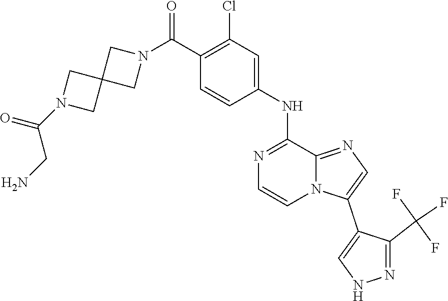 [SMILES: NCC(=O)N1CC2(C1)CN(C(=O)c1ccc(Nc3nccn4c(-c5c[nH]nc5C(F)(F)F)cnc34)cc1Cl)C2] | 2-amino-1-(6-(2- chloro-4-((3-(3- (trifluoromethyl)- 1H-pyrazol-4- yl)imidazo[1,2-a]pyrazin-8- yl)amino)benzoyl)-2,6- diazaspiro[3.3]heptan- 2-yl)ethan-1- one | 559.9 | 558.3 [M − H]−, ESI neg |
| 659 | 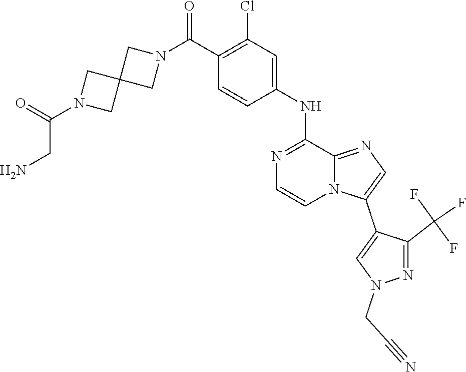 [SMILES: N#CCn1cc(-c2cnc3c(Nc4ccc(C(=O)N5CC6(CN(C(=O)CN)C6)C5)c(Cl)c4)nccn23)c(C(F)(F)F)n1] | 2-(4-(8-((3-chloro-4- (6-glycyl-2,6- diazaspiro[3.3]heptane-2- carbonyl)phenyl)amino) imidazo[1,2- a]pyrazin-3-yl)-3- (trifluoromethyl)-1H- pyrazol-1- yl)acetonitrile | 599.0 | 597.4 [M − H]−, ESI neg |
| 660 | 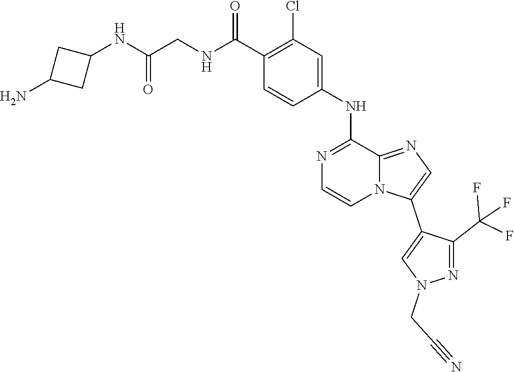 [SMILES: N#CCn1cc(-c2cnc3c(Nc4ccc(C(=O)NCC(=O)NC5CC(N)C5)c(Cl)c4)nccn23)c(C(F)(F)F)n1] | N-[2-[(3-aminocyclobutyl) amino]- 2-oxo-ethyl]-2- chloro-4-[[3-[1- (cyanomethyl)-3- (trifluoromethyl)pyrazol-4- yl]imidazo[1,2-a] pyrazin-8- yl]amino] benzamide formate | 587.0 | 587.1 [M − H]−, ESI neg |
| 661 | 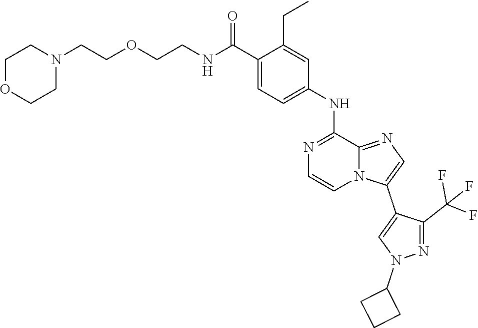 [SMILES: CCc1cc(Nc2nccn3c(-c4cn(C5CCC5)nc4C(F)(F)F)cnc23)ccc1C(=O)NCCOCCN1CCOCC1] | 4-((3-(1-cyclobutyl-3- (trifluoromethyl)- 1H-pyrazol-4- yl)imidazo[1,2-a]pyrazin-8- yl)amino)-2-ethyl-N-(2-(2- morpholinoethoxy)ethyl) benzamide formate | 626.7 | 627.4 |
| 662 | 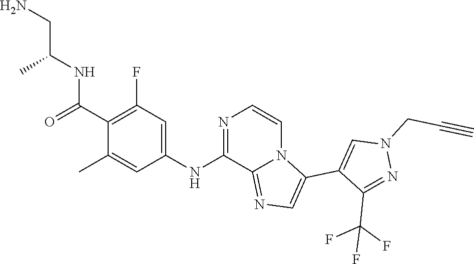 [SMILES: C#CCn1cc(-c2cnc3c(Nc4cc(C)c(C(=O)N[C@H](C)CN)c(F)c4)nccn23)c(C(F)(F)F)n1] | (R)-N-(1-aminopropan- 2-yl)-2- fluoro-6-methyl-4- ((3-(1-(prop-2- yn-1-yl)-3- (trifluoromethyl)-1H- pyrazol-4-yl)imidazo [1,2-a]pyrazin- 8-yl)amino)benzamide | 514.5 | 515.3 |
| 663 | 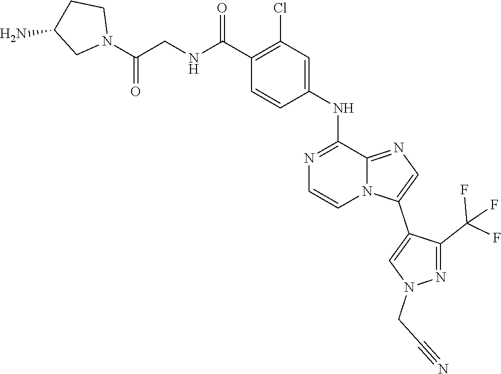 [SMILES: N#CCn1cc(-c2cnc3c(Nc4ccc(C(=O)NCC(=O)N5CC[C@@H](N)C5)c(Cl)c4)nccn23)c(C(F)(F)F)n1] | N-[2-[(3R)-3- aminopyrrolidin-1- yl]-2-oxo-ethyl]-2- chloro-4-[[3-[1- (cyanomethyl)-3- (trifluoromethyl)pyrazol-4- yl]imidazo[1,2-a]pyrazin-8- yl]amino]benzamide formate | 587.0 | 587 |
| 664 | 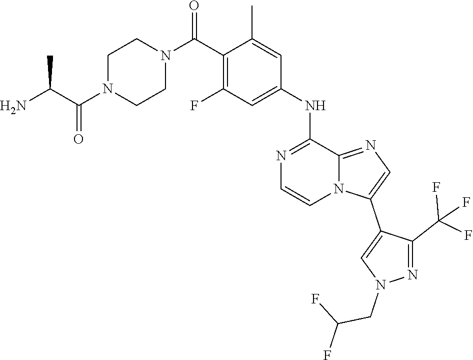 [SMILES: Cc1cc(Nc2nccn3c(-c4cn(CC(F)F)nc4C(F)(F)F)cnc23)cc(F)c1C(=O)N1CCN(C(=O)[C@H](C)N)CC1] | (S)-2-amino-1-(4- (4-((3-(1-(2,2- difluoroethyl)-3- (trifluoromethyl)- 1H-pyrazol-4-yl)imidazo [1,2-a]pyrazin-8-yl)amino)- 2-fluoro-6- methylbenzoyl)piperazin-1- yl)propan-1-one | 623.6 | 624.4 |
| 665 | 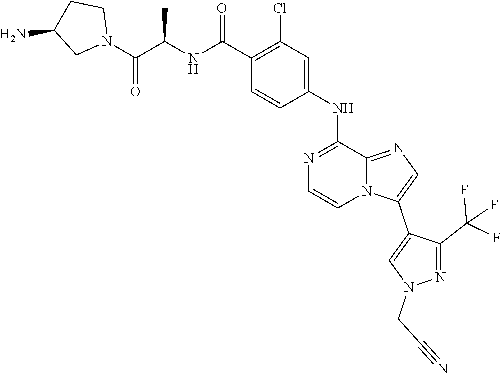 [SMILES: C[C@@H](NC(=O)c1ccc(Nc2nccn3c(-c4cn(CC#N)nc4C(F)(F)F)cnc23)cc1Cl)C(=O)N1CC[C@H](N)C1] | N-[(1R)-2-[(3S)-3- aminopyrrolidin- 1-yl]-1-methyl-2- oxo-ethyl]-2- chloro-4-[[3-[1- (cyanomethyl)-3- (trifluoromethyl)pyrazol-4- yl]imidazo[1,2-a]pyrazin-8- yl]amino]benzamide formate | 601.0 | 601.1 |
| 666 | 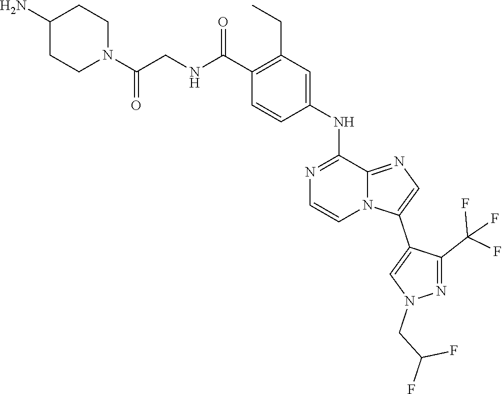 [SMILES: CCc1cc(Nc2nccn3c(-c4cn(CC(F)F)nc4C(F)(F)F)cnc23)ccc1C(=O)NCC(=O)N1CCC(N)CC1] | N-[2-(4-amino-1- piperidyl)-2-oxo- ethyl]-4-[[3-[1-(2,2- difluoroethyl)- 3-(trifluoromethyl) pyrazol-4- yl]imidazo[1,2-a] pyrazin-8- yl]amino]-2-ethyl- benzamide formate | 619.6 | 620.3 |
| 667 | 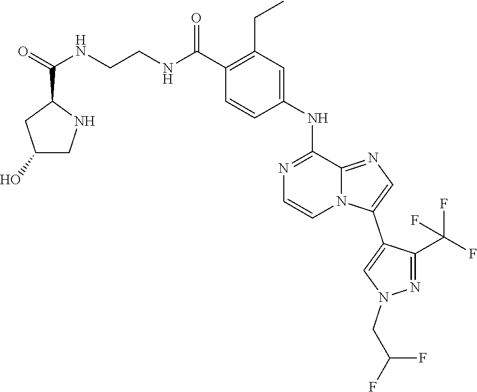 [SMILES: CCc1cc(Nc2nccn3c(-c4cn(CC(F)F)nc4C(F)(F)F)cnc23)ccc1C(=O)NCCNC(=O)[C@@H]1C[C@@H](O)CN1] | (2S,4R)-N-(2-(4 -((3-(1-(2,2- difluoroethyl)-3- (trifluoromethyl)- 1H-pyrazol-4-yl) imidazo[1,2- a]pyrazin-8-yl)amino)-2- ethylbenzamido)ethyl)-4- hydroxypyrrolidine-2- carboxamide | 635.6 | 636.3 |
| 668 | 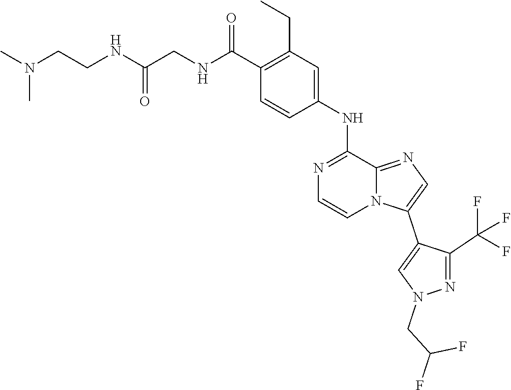 [SMILES: CCc1cc(Nc2nccn3c(-c4cn(CC(F)F)nc4C(F)(F)F)cnc23)ccc1C(=O)NCC(=O)NCCN(C)C] | 4-[[3-[1-(2,2- difluoroethyl)-3- (trifluoromethyl) pyrazol-4- yl]imidazo[1,2-a]pyrazin- 8-yl]amino]-N-[2-[2- (dimethylamino) ethylamino]-2-oxo- ethyl]-2-ethyl- benzamide formate | 607.6 | 608.2 |
| 669 | 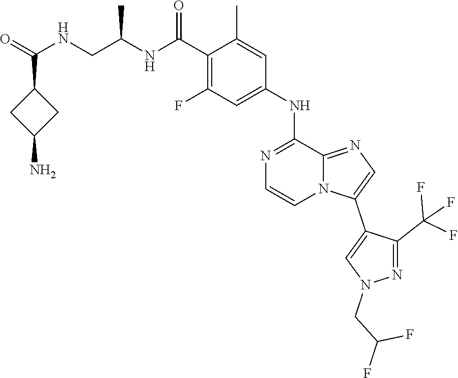 [SMILES: Cc1cc(Nc2nccn3c(-c4cn(CC(F)F)nc4C(F)(F)F)cnc23)cc(F)c1C(=O)N[C@H](C)CNC(=O)[C@H]1C[C@@H](N)C1] | N-((R)-1-((1s,3S)-3- aminocyclobutane-1- carboxamido)propan- 2-yl)-4-((3-(1- (2,2-difluoroethyl)-3- (trifluoromethyl)- 1H-pyrazol-4- yl)imidazo[1,2-a]pyrazin- 8-yl)amino)-2-fluoro-6- methylbenzamide | 637.6 | 638.4 |
| 670 | 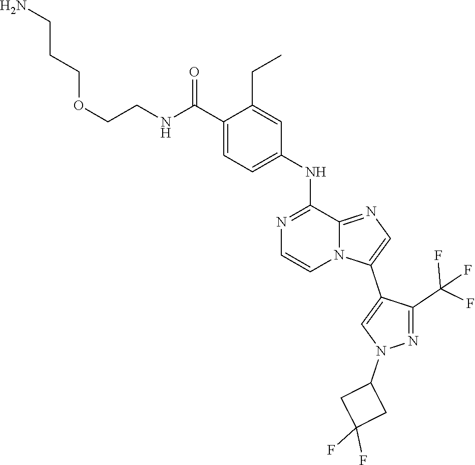 [SMILES: CCc1cc(Nc2nccn3c(-c4cn(C5CC(F)(F)C5)nc4C(F)(F)F)cnc23)ccc1C(=O)NCCOCCCN] | N-(2-(3-aminopropoxy) ethyl)-4-((3-(1-(3,3- difluorocyclobutyl)-3- (trifluoromethyl)- 1H-pyrazol-4- yl)imidazo[1,2-a] pyrazin-8- yl)amino)-2- ethylbenzamide formate | 606.6 | 607.3 |
| 671 | 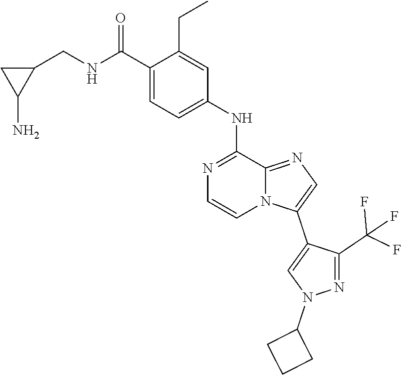 [SMILES: CCc1cc(Nc2nccn3c(-c4cn(C5CCC5)nc4C(F)(F)F)cnc23)ccc1C(=O)NCC1CC1N] | N-((2-aminocyclopropyl) methyl)-4- ((3-(1-cyclobutyl-3- (trifluoromethyl)- 1H-pyrazol-4- yl)imidazo[1,2-a]pyrazin-8- yl)amino)-2- ethylbenzamide formate | 538.6 | 539.3 |
| 672 | 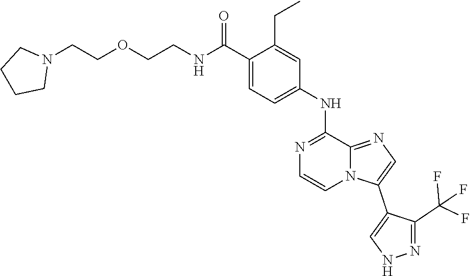 [SMILES: CCc1cc(Nc2nccn3c(-c4c[nH]nc4C(F)(F)F)cnc23)ccc1C(=O)NCCOCCN1CCCC1] | 2-ethyl-N-(2-(2- (pyrrolidin-1- yl)ethoxy)ethyl)-4-((3-(3- (trifluoromethyl)- 1H-pyrazol-4- yl)imidazo[1,2-a] pyrazin-8- yl)amino)benzamide | 556.6 | 557.3 |
| 673 | 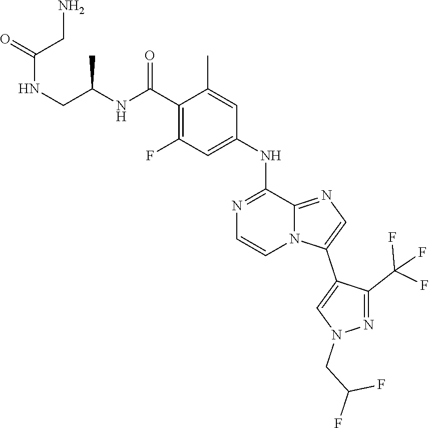 [SMILES: Cc1cc(Nc2nccn3c(-c4cn(CC(F)F)nc4C(F)(F)F)cnc23)cc(F)c1C(=O)N[C@H](C)CNC(=O)CN] | (R)-N-(1-(2- aminoacetamido) propan-2-yl)-4- ((3-(1-(2,2-difluoroethyl)-3- (trifluoromethyl)- 1H-pyrazol-4- yl)imidazo[1,2-a]pyrazin- 8-yl)amino)-2-fluoro-6- methylbenzamide | 597.5 | 598.3 |
| 674 | 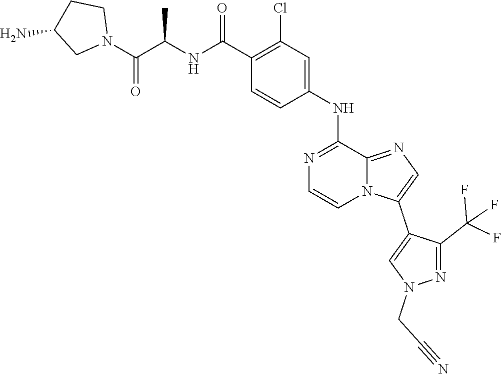 [SMILES: C[C@@H](NC(=O)c1ccc(Nc2nccn3c(-c4cn(CC#N)nc4C(F)(F)F)cnc23)cc1Cl)C(=O)N1CC[C@@H](N)C1] | N-[(1R)-2-[(3R)-3- aminopyrrolidin- 1-yl]-1-methyl-2- oxo-ethyl]-2- chloro-4-[[3-[1- (cyanomethyl)-3- (trifluoromethyl) pyrazol-4- yl]imidazo[1,2-a]pyrazin- 8-yl]amino] benzamide formate | 601.0 | 601.1 |
| 675 | 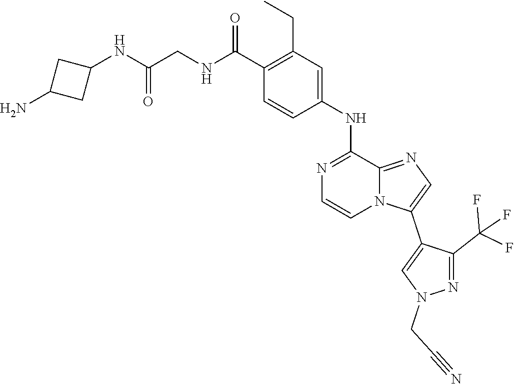 [SMILES: CCc1cc(Nc2nccn3c(-c4cn(CC#N)nc4C(F)(F)F)cnc23)ccc1C(=O)NCC(=O)NC1CC(N)C1] | N-[2-[(3-aminocyclobutyl) amino]- 2-oxo-ethyl]-4-[[3-[1- (cyanomethyl)-3- (trifluoromethyl)pyrazol-4- yl]imidazo[1,2-a]pyrazin- 8-yl]amino]-2-ethyl- benzamide formate | 580.6 | 581.1 |
| 676 | 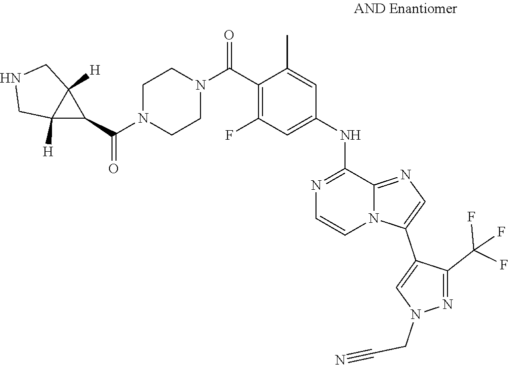 [SMILES: Cc1cc(Nc2nccn3c(-c4cn(CC#N)nc4C(F)(F)F)cnc23)cc(F)c1C(=O)N1CCN(C(=O)[C@H]2[C@@H]3CNC[C@@H]32)CC1] | 2-(4-(8-((4-(4- ((1R,5S,6r)-3- azabicyclo[3.1.0]hexane- 6-carbonyl)piperazine-1- carbonyl)-3- fluoro-5- methylphenyl)amino) imidazo[1,2- a]pyrazin-3-yl)-3- (trifluoromethyl)- 1H-pyrazol-1-yl) acetonitrile formate | 636.6 | 637.4 |
| 677 | 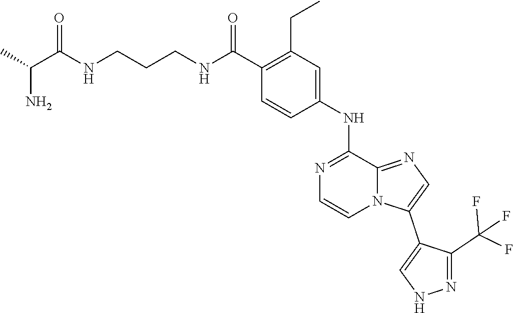 [SMILES: CCc1cc(Nc2nccn3c(-c4c[nH]nc4C(F)(F)F)cnc23)ccc1C(=O)NCCCNC(=O)[C@@H](C)N] | (R)-N-(3-(2- aminopropanamido) propyl)-2-ethyl- 4-((3-(3-(trifluoromethyl)- 1H-pyrazol-4-yl)imidazo [1,2-a]pyrazin- 8-yl)amino)benzamide formate | 543.6 | 542.4 [M − H]−, ESI neg. |
| 678 | 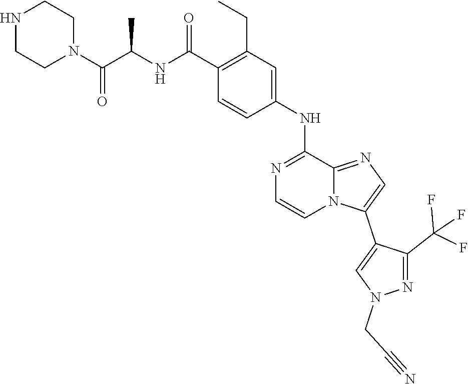 [SMILES: CCc1cc(Nc2nccn3c(-c4cn(CC#N)nc4C(F)(F)F)cnc23)ccc1C(=O)N[C@H](C)C(=O)N1CCNCC1] | 4-[[3-[1-(cyanomethyl)-3- (trifluoromethyl)pyrazol-4- yl]imidazo[1,2-a]pyrazin-8- yl]amino]-2-ethyl- N-[(1R)-1- methyl-2-oxo-2- piperazin-1-yl- ethyl]benzamide formate | 594.6 | 595.1 |
| 679 | 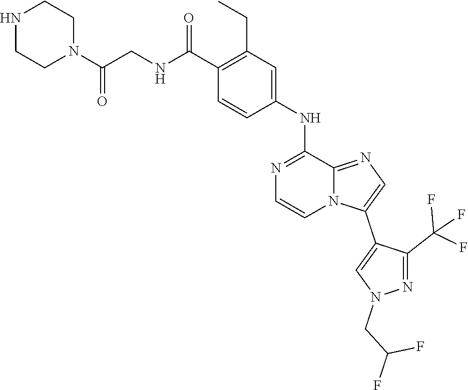 [SMILES: CCc1cc(Nc2nccn3c(-c4cn(CC(F)F)nc4C(F)(F)F)cnc23)ccc1C(=O)NCC(=O)N1CCNCC1] | 4-[[3-[1-(2,2- difluoroethyl)-3- (trifluoromethyl)pyrazol-4- yl]imidazo[1,2-a] pyrazin-8- yl]amino]-2-ethyl- N-(2-oxo-2- piperazin-1-yl-ethyl) benzamide formate | 605.6 | 606.3 |
| 680 | 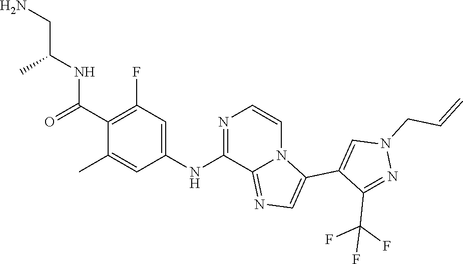 [SMILES: C=CCn1cc(-c2cnc3c(Nc4cc(C)c(C(=O)N[C@H](C)CN)c(F)c4)nccn23)c(C(F)(F)F)n1] | (R)-4-((3-(1-allyl-3- (trifluoromethyl)- 1H-pyrazol-4- yl)imidazo[1,2-a] pyrazin-8- yl)amino)-N-(1- aminopropan-2-yl)- 2-fluoro-6- methylbenzamide | 516.5 | 517.3 |
| 681 | 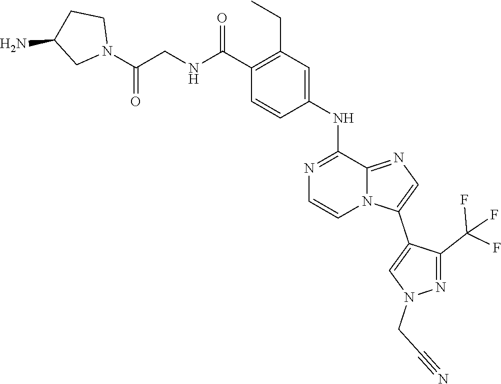 [SMILES: CCc1cc(Nc2nccn3c(-c4cn(CC#N)nc4C(F)(F)F)cnc23)ccc1C(=O)NCC(=O)N1CC[C@H](N)C1] | N-[2-[(3S)-3- aminopyrrolidin-1-yl]- 2-oxo-ethyl]-4-[[3-[1- (cyanomethyl)-3- (trifluoromethyl) pyrazol-4- yl]imidazo[1,2-a]pyrazin-8- yl]amino]-2-ethyl- benzamide formate | 580.6 | 581.2 |
| 682 | 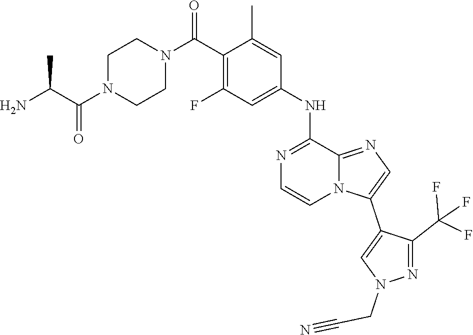 [SMILES: Cc1cc(Nc2nccn3c(-c4cn(CC#N)nc4C(F)(F)F)cnc23)cc(F)c1C(=O)N1CCN(C(=O)[C@H](C)N)CC1] | 2-(4-(8-((4-(4-(L-alanyl) piperazine- 1-carbonyl)-3-fluoro-5- methylphenyl)amino) imidazo[1,2- a]pyrazin-3-yl)-3- (trifluoromethyl)- 1H-pyrazol-1-yl) acetonitrile formate | 598.6 | 599.4 |
| 683 | 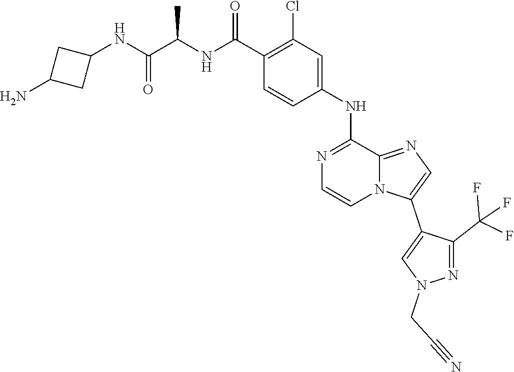 [SMILES: C[C@@H](NC(=O)c1ccc(Nc2nccn3c(-c4cn(CC#N)nc4C(F)(F)F)cnc23)cc1Cl)C(=O)NC1CC(N)C1] | N-[(1R)-2-[(3- aminocyclobutyl)amino]- 1-methyl- 2-oxo-ethyl]-2-chloro- 4-[[3-[1- (cyanomethyl)-3- (trifluoromethyl)pyrazol-4- yl]imidazo[1,2-a]pyrazin-8- yl]amino]benzamide formate | 601.0 | 601.1 |
| 684 | 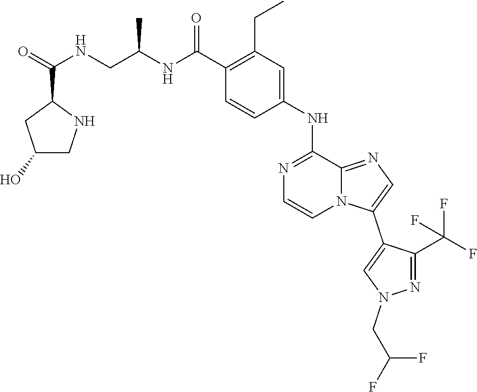 [SMILES: CCc1cc(Nc2nccn3c(-c4cn(CC(F)F)nc4C(F)(F)F)cnc23)ccc1C(=O)N[C@H](C)CNC(=O)[C@@H]1C[C@@H](O)CN1] | (2S,4R)-N-((R)-2- (4-((3-(1-(2,2- difluoroethyl)-3- (trifluoromethyl)- 1H-pyrazol-4-yl) imidazo[1,2- a]pyrazin-8-yl)amino)-2- ethylbenzamido)propyl)-4- hydroxypyrrolidine-2- carboxamide | 649.6 | 650.4 |
| 685 | 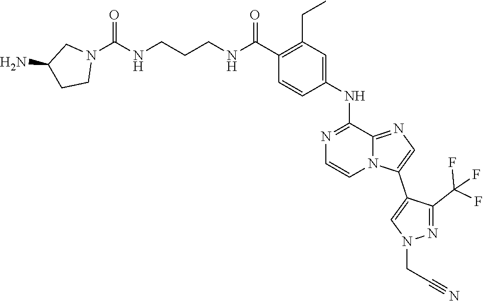 [SMILES: CCc1cc(Nc2nccn3c(-c4cn(CC#N)nc4C(F)(F)F)cnc23)ccc1C(=O)NCCCNC(=O)N1CC[C@@H](N)C1] | (R)-3-amino-N-(3- (4-((3-(1- (cyanomethyl)-3- (trifluoromethyl)- 1H-pyrazol-4-yl) imidazo[1,2- a]pyrazin-8-yl)amino)-2- ethylbenzamido)propyl) pyrrolidine- 1-carboxamide formate | 623.6 | 668.6 [M + HCOO]−, ESI neg. |
| 686 | 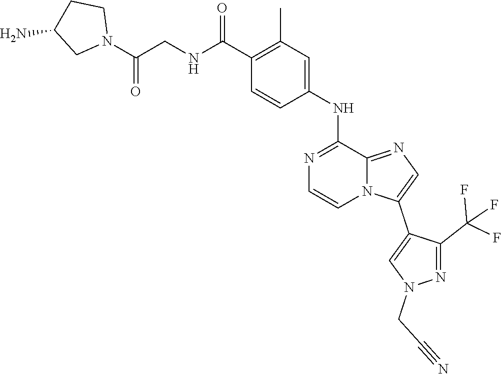 [SMILES: Cc1cc(Nc2nccn3c(-c4cn(CC#N)nc4C(F)(F)F)cnc23)ccc1C(=O)NCC(=O)N1CC[C@@H](N)C1] | N-[2-[(3R)-3- aminopyrrolidin-1- yl]-2-oxo-ethyl]-4-[[3-[1- (cyanomethyl)-3- (trifluoromethyl) pyrazol-4- yl]imidazo[1,2-a] pyrazin-8- yl]amino]-2-methyl- benzamide | 566.5 | 567.2 |
| 687 | 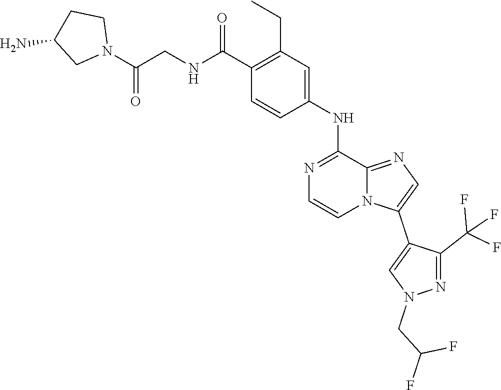 [SMILES: CCc1cc(Nc2nccn3c(-c4cn(CC(F)F)nc4C(F)(F)F)cnc23)ccc1C(=O)NCC(=O)N1CC[C@@H](N)C1] | N-[2-[(3R)-3- aminopyrrolidin-1- yl]-2-oxo-ethyl]-4- [[3-[1-(2,2- difluoroethyl)-3- (trifluoromethyl)pyrazol-4- yl]imidazo[1,2-a]pyrazin-8- yl]amino]-2-ethyl- benzamideformate | 605.6 | 606.1 |
| 688 | 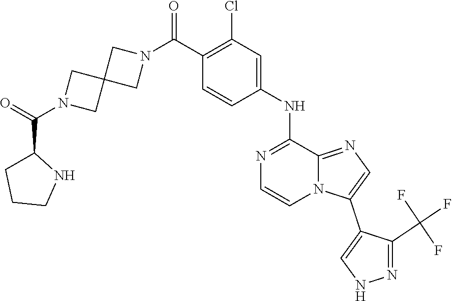 [SMILES: O=C(c1ccc(Nc2nccn3c(-c4c[nH]nc4C(F)(F)F)cnc23)cc1Cl)N1CC2(C1)CN(C(=O)[C@@H]1CCCN1)C2] | (S)-(2-chloro-4-((3-(3- (trifluoromethyl)- 1H-pyrazol-4- yl)imidazo[1,2-a]pyrazin- 8-yl)amino)phenyl)(6- prolyl-2,6- diazaspiro[3.3]heptan-2- yl)methanone | 600.0 | 598.4 [M − H]−, ESI neg. |
| 689 | 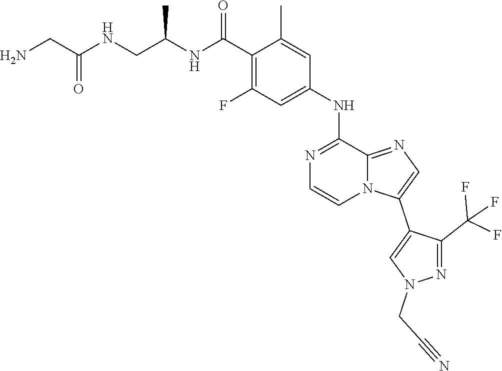 [SMILES: Cc1cc(Nc2nccn3c(-c4cn(CC#N)nc4C(F)(F)F)cnc23)cc(F)c1C(=O)N[C@H](C)CNC(=O)CN] | (R)-N-(1-(2- aminoacetamido) propan-2-yl)-4- ((3-(1-(cyanomethyl)-3- (trifluoromethyl)- 1H-pyrazol-4- yl)imidazo[1,2-a]pyrazin- 8-yl)amino)-2-fluoro-6- methylbenzamide formate | 572.5 | 573.4 |
| 690 | 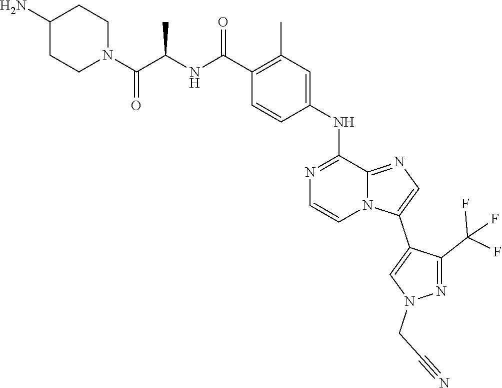 [SMILES: Cc1cc(Nc2nccn3c(-c4cn(CC#N)nc4C(F)(F)F)cnc23)ccc1C(=O)N[C@H](C)C(=O)N1CCC(N)CC1] | N-[(1R)-2-(4-amino- 1-piperidyl)-1- methyl-2-oxo-ethyl]- 4-[[3-[1- (cyanomethyl)-3- (trifluoromethyl) pyrazol-4- yl]imidazo[1,2-a] pyrazin-8- yl]amino]-2-methyl- benzamide formate | 594.6 | 595.3 |
| 691 | 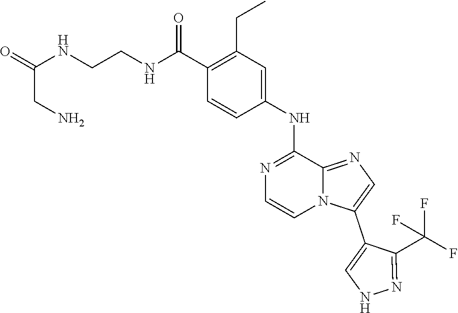 [SMILES: CCc1cc(Nc2nccn3c(-c4c[nH]nc4C(F)(F)F)cnc23)ccc1C(=O)NCCNC(=O)CN] | N-(2-(2- aminoacetamido) ethyl)-2- ethyl-4-((3-(3- (trifluoromethyl)-1H- pyrazol-4-yl)imidazo [1,2-a]pyrazin- 8-yl)amino)benzamide formate | 515.5 | 516.3 |
| 692 | 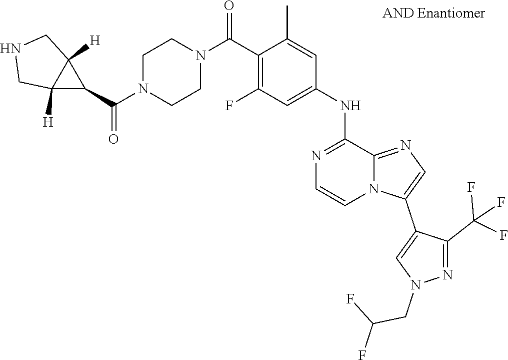 [SMILES: Cc1cc(Nc2nccn3c(-c4cn(CC(F)F)nc4C(F)(F)F)cnc23)cc(F)c1C(=O)N1CCN(C(=O)[C@H]2[C@@H]3CNC[C@@H]32)CC1] | (4-((1R,5S,6r)-3- azabicyclo[3.1.0]hexane- 6-carbonyl)piperazin- 1-yl)(4-((3-(1- (2,2-difluoroethyl)-3- (trifluoromethyl)- 1H-pyrazol-4- yl)imidazo[1,2-a]pyrazin-8- yl)amino)-2-fluoro-6- methylphenyl)methanone | 661.6 | 662.4 |
| 693 | 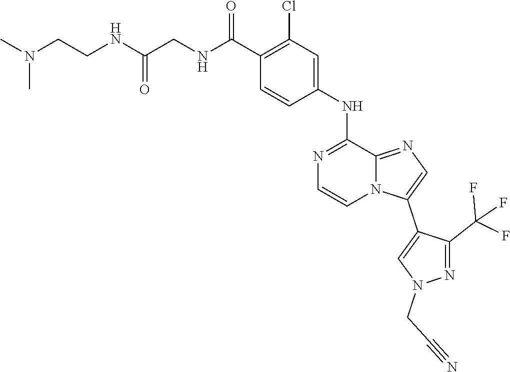 [SMILES: CN(C)CCNC(=O)CNC(=O)c1ccc(Nc2nccn3c(-c4cn(CC#N)nc4C(F)(F)F)cnc23)cc1Cl] | 2-chloro-4-[[3-[1- (cyanomethyl)-3- (trifluoromethyl)pyrazol-4- yl]imidazo[1,2-a]pyrazin-8- yl]amino]-N-[2-[2- (dimethylamino) ethylamino]-2-oxo- ethyl]benzamide formate | 589.0 | 589.3 |
| 694 | 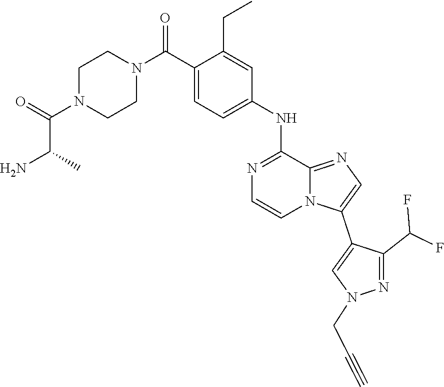 [SMILES: C#CCn1cc(-c2cnc3c(Nc4ccc(C(=O)N5CCN(C(=O)[C@H](C)N)CC5)c(CC)c4)nccn23)c(C(F)F)n1] | (S)-2-amino-1-(4- (4-((3-(3- (difluoromethyl)-1- (prop-2-yn-1- yl)-1H-pyrazol-4-yl) imidazo[1,2- a]pyrazin-8-yl)amino)-2- ethylbenzoyl)piperazin-1- yl)propan-1-one | 575.6 | 576.3 |
| 695 | 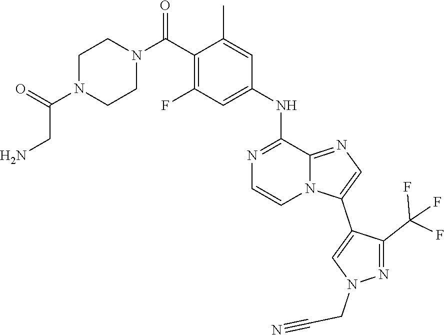 [SMILES: Cc1cc(Nc2nccn3c(-c4cn(CC#N)nc4C(F)(F)F)cnc23)cc(F)c1C(=O)N1CCN(C(=O)CN)CC1] | 2-(4-(8-((3-fluoro-4-(4- glycylpiperazine- 1-carbonyl)-5- methylphenyl)amino) imidazo[1,2- a]pyrazin-3-yl)-3- (trifluoromethyl)- 1H-pyrazol-1-yl) acetonitrile formate | 584.5 | 585.3 |
| 696 | 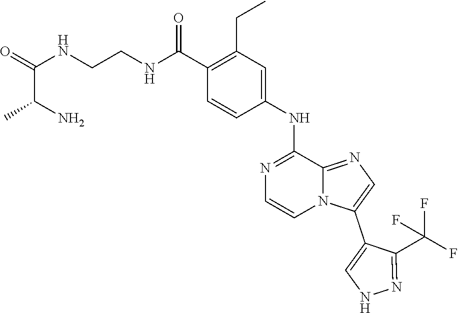 [SMILES: CCc1cc(Nc2nccn3c(-c4c[nH]nc4C(F)(F)F)cnc23)ccc1C(=O)NCCNC(=O)[C@@H](C)N] | (R)-N-(2-(2- aminopropanamido) ethyl)-2-ethyl- 4-((3-(3-(trifluoromethyl)- 1H-pyrazol-4-yl)imidazo [1,2-a]pyrazin- 8-yl)amino)benzamide formate | 529.5 | 530.3 |
| 697 | 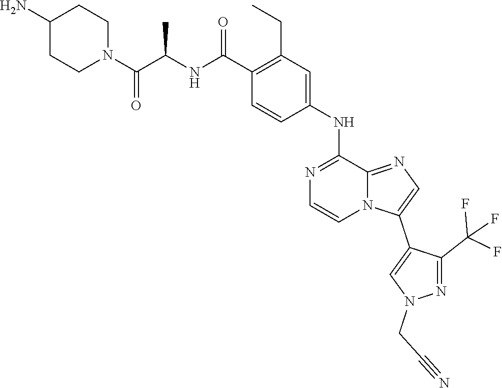 [SMILES: CCc1cc(Nc2nccn3c(-c4cn(CC#N)nc4C(F)(F)F)cnc23)ccc1C(=O)N[C@H](C)C(=O)N1CCC(N)CC1] | N-[(1R)-2-(4-amino- 1-piperidyl)-1- methyl-2-oxo-ethyl]- 4-[[3-[1- (cyanomethyl)-3- (trifluoromethyl)pyrazol-4- yl]imidazo[1,2-a]pyrazin- 8-yl]amino]-2-ethyl- benzamide formate | 608.6 | 609.2 |
| 698 | 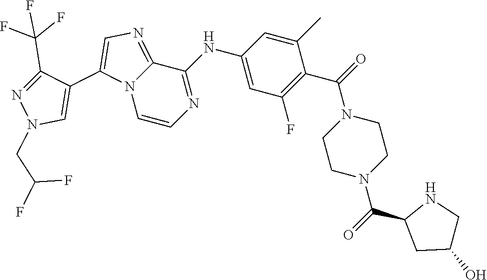 [SMILES: Cc1cc(Nc2nccn3c(-c4cn(CC(F)F)nc4C(F)(F)F)cnc23)cc(F)c1C(=O)N1CCN(C(=O)[C@@H]2C[C@@H](O)CN2)CC1] | (4-(4-((3-(1-(2,2- difluoroethyl)-3- (trifluoromethyl)- 1H-pyrazol-4- yl)imidazo[1,2-a]pyrazin-8- yl)amino)-2-fluoro-6- methylbenzoyl)piperazin-1- yl)((2S,4R)-4- hydroxypyrrolidin-2- yl)methanone | 665.6 | 666.4 |
| 699 | 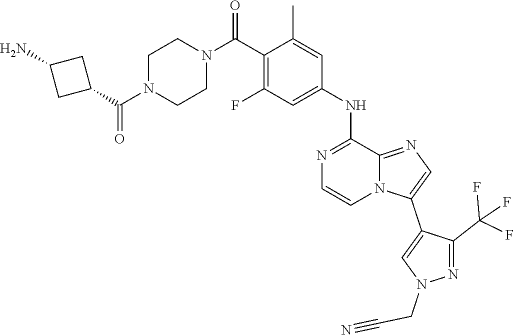 [SMILES: Cc1cc(Nc2nccn3c(-c4cn(CC#N)nc4C(F)(F)F)cnc23)cc(F)c1C(=O)N1CCN(C(=O)[C@H]2C[C@@H](N)C2)CC1] | 2-(4-(8-((4-(4-((1s,3s)-3- aminocyclobutane-1- carbonyl)piperazine- 1-carbonyl)-3- fluoro-5- methylphenyl)amino) imidazo[1,2- a]pyrazin-3-yl)-3- (trifluoromethyl)- 1H-pyrazol-1-yl) acetonitrile formate | 624.6 | 625.3 |
| 700 | 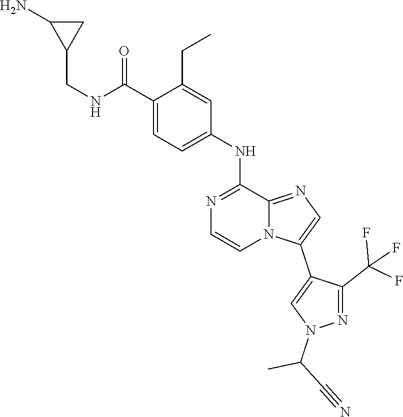 [SMILES: CCc1cc(Nc2nccn3c(-c4cn(C(C)C#N)nc4C(F)(F)F)cnc23)ccc1C(=O)NCC1CC1N] | N-((2-aminocyclopropyl) methyl)-4- ((3-(1-(1-cyanoethyl)-3- (trifluoromethyl)- 1H-pyrazol-4- yl)imidazo[1,2-a] pyrazin-8- yl)amino)-2- ethylbenzamide formate | 537.5 | 538.3 |
| 701 | 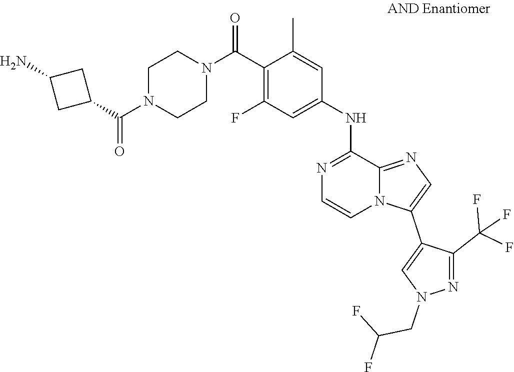 [SMILES: Cc1cc(Nc2nccn3c(-c4cn(CC(F)F)nc4C(F)(F)F)cnc23)cc(F)c1C(=O)N1CCN(C(=O)[C@H]2C[C@@H](N)C2)CC1] | (4-((1s,3s)-3- aminocyclobutane-1- carbonyl)piperazin- 1-yl)(4-((3-(1- (2,2-difluoroethyl)-3- (trifluoromethyl)- 1H-pyrazol-4- yl)imidazo[1,2-a]pyrazin-8- yl)amino)-2-fluoro-6- methylphenyl)methanone | 649.6 | 650.4 |
| 702 | 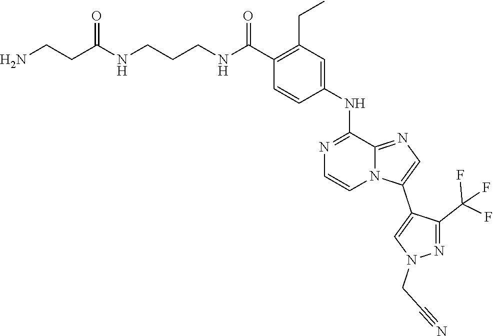 [SMILES: CCc1cc(Nc2nccn3c(-c4cn(CC#N)nc4C(F)(F)F)cnc23)ccc1C(=O)NCCCNC(=O)CCN] | N-(3-(3- aminopropanamido) propyl)-4-((3- (1-(cyanomethyl)-3- (trifluoromethyl)-1H- pyrazol-4- yl)imidazo[1,2-a] pyrazin-8- yl)amino)-2- ethylbenzamide formate | 582.6 | 627.5 [M + HCOO]−, ESI neg |
| 703 | 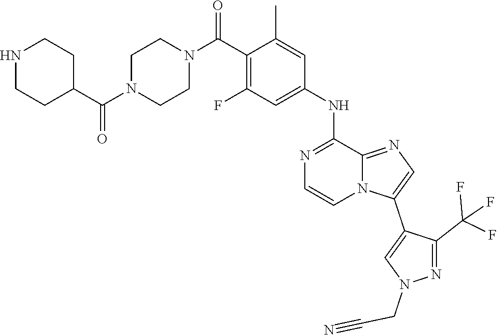 [SMILES: Cc1cc(Nc2nccn3c(-c4cn(CC#N)nc4C(F)(F)F)cnc23)cc(F)c1C(=O)N1CCN(C(=O)C2CCNCC2)CC1] | 2-(4-(8-((3-fluoro- 5-methyl-4-(4- (piperidine-4- carbonyl)piperazine-1- carbonyl)phenyl) amino)imidazo[1,2- a]pyrazin-3-yl)-3- (trifluoromethyl)- 1H-pyrazol-1- yl)acetonitrile formate | 638.6 | 639.4 |
| 704 | 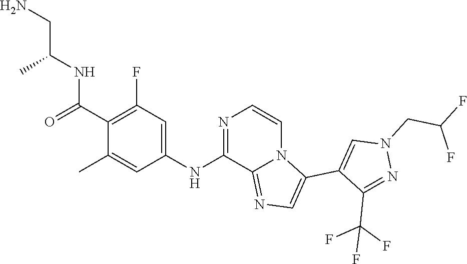 [SMILES: Cc1cc(Nc2nccn3c(-c4cn(CC(F)F)nc4C(F)(F)F)cnc23)cc(F)c1C(=O)N[C@H](C)CN] | (R)-N-(1-aminopropan- 2-yl)-4-((3- (1-(2,2-difluoroethyl)-3- (trifluoromethyl)-1H- pyrazol-4- yl)imidazo[1,2-a]pyrazin- 8-yl)amino)-2-fluoro-6- methylbenzamide | 540.5 | 541.3 |
| 705 | 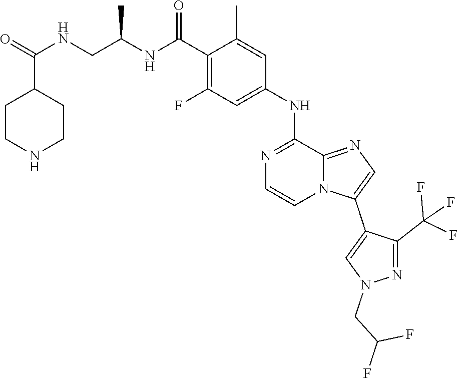 [SMILES: Cc1cc(Nc2nccn3c(-c4cn(CC(F)F)nc4C(F)(F)F)cnc23)cc(F)c1C(=O)N[C@H](C)CNC(=O)C1CCNCC1] | (R)-N-(2-(4-((3-(1-(2,2- difluoroethyl)-3- (trifluoromethyl)- 1H-pyrazol-4-yl) imidazo[1,2- a]pyrazin-8-yl)amino)- 2-fluoro-6- methylbenzamido) propyl)piperidine- 4-carboxamide | 651.6 | 652.4 |
| 706 | 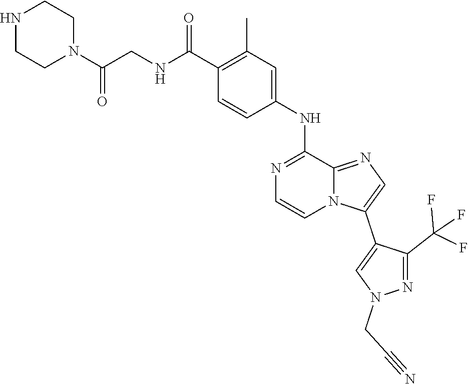 [SMILES: Cc1cc(Nc2nccn3c(-c4cn(CC#N)nc4C(F)(F)F)cnc23)ccc1C(=O)NCC(=O)N1CCNCC1] | 4-[[3-[1-(cyanomethyl)-3- (trifluoromethyl)pyrazol-4- yl]imidazo[1,2-a] pyrazin-8- yl]amino]-2-methyl- N-(2-oxo-2- piperazin-1-yl-ethyl) benzamide formate | 566.5 | 567.3 |
| 707 | 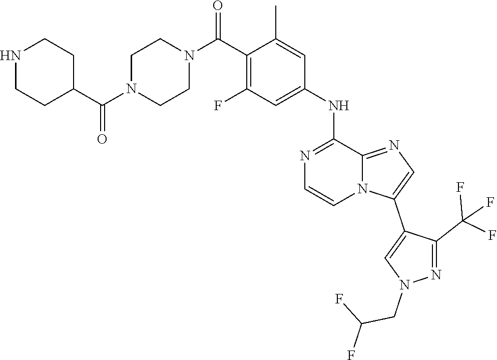 [SMILES: Cc1cc(Nc2nccn3c(-c4cn(CC(F)F)nc4C(F)(F)F)cnc23)cc(F)c1C(=O)N1CCN(C(=O)C2CCNCC2)CC1] | (4-(4-((3-(1-(2,2- difluoroethyl)-3- (trifluoromethyl)- 1H-pyrazol-4- yl)imidazo[1,2-a]pyrazin- 8-yl)amino)-2-fluoro-6- methylbenzoyl) piperazin-1- yl)(piperidin-4-yl) methanone | 663.6 | 664.4 |
| 708 | 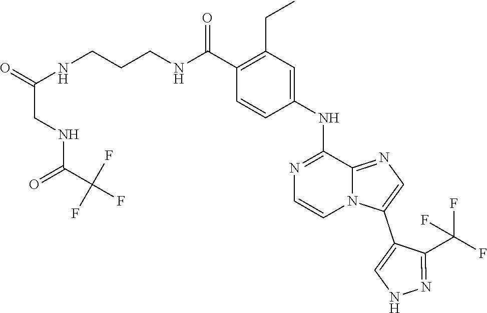 [SMILES: CCc1cc(Nc2nccn3c(-c4c[nH]nc4C(F)(F)F)cnc23)ccc1C(=O)NCCCNC(=O)CNC(=O)C(F)(F)F] | 2-ethyl-N-(3-(2-(2,2,2- trifluoroacetamido) acetamido)propyl)- 4-((3-(3-(trifluoromethyl)- 1H-pyrazol-4-yl)imidazo [1,2-a]pyrazin- 8-yl)amino)benzamide | 625.5 | 626.3 |
| 709 | 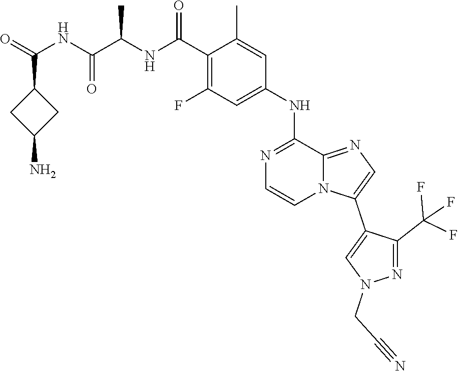 [SMILES: Cc1cc(Nc2nccn3c(-c4cn(CC#N)nc4C(F)(F)F)cnc23)cc(F)c1C(=O)N[C@H](C)C(=O)NC(=O)[C@H]1C[C@@H](N)C1] | N-((R)-1-((1s,3S)-3- aminocyclobutane-1- carboxamido)propan- 2-yl)-4-((3-(1- (cyanomethyl)-3- (trifluoromethyl)- 1H-pyrazol-4-y l)imidazo[1,2- a]pyrazin-8-yl)amino)- 2-fluoro-6- methylbenzamide formate | 612.6 | 613.4 |
| 710 | 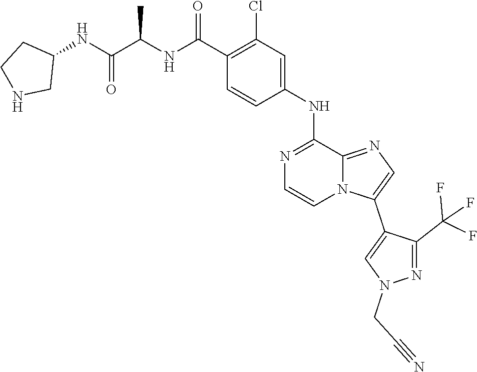 [SMILES: C[C@@H](NC(=O)c1ccc(Nc2nccn3c(-c4cn(CC#N)nc4C(F)(F)F)cnc23)cc1Cl)C(=O)N[C@H]1CCNC1] | 2-chloro-4-[[3-[1- (cyanomethyl)-3- (trifluoromethyl)pyrazol-4- yl]imidazo[1,2-a]pyrazin-8- yl]amino]-N-[(1R)-1- methyl-2-oxo- 2-[[(3S)-pyrrolidin-3- yl]amino]ethyl] benzamide formate | 601.0 | 601.3 |
| 711 | 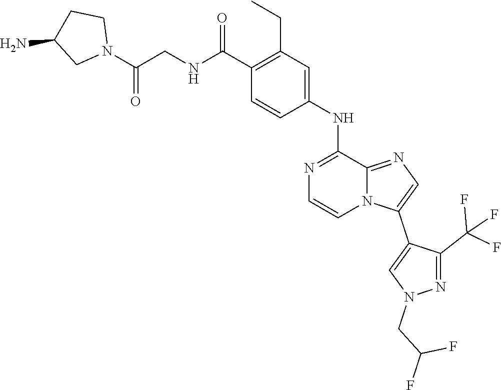 [SMILES: CCc1cc(Nc2nccn3c(-c4cn(CC(F)F)nc4C(F)(F)F)cnc23)ccc1C(=O)NCC(=O)N1CC[C@H](N)C1] | N-[2-[(3S)-3- aminopyrrolidin-1-yl]- 2-oxo-ethyl]-4-[[3-[1-(2,2- difluoroethyl)-3- (trifluoromethyl)pyrazol-4- yl]imidazo[1,2-a]pyrazin-8- yl]amino]-2-ethyl- benzamide | 605.6 | 606.1 |
| 712 | 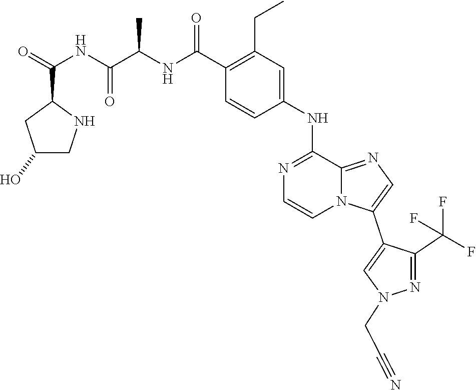 [SMILES: CCc1cc(Nc2nccn3c(-c4cn(CC#N)nc4C(F)(F)F)cnc23)ccc1C(=O)N[C@H](C)C(=O)NC(=O)[C@@H]1C[C@@H](O)CN1] | (2S,4R)-N-((R)-2- (4-((3-(1- (cyanomethyl)-3- (trifluoromethyl)- 1H-pyrazol-4-yl) imidazo[1,2- a]pyrazin-8-yl)amino)-2- ethylbenzamido)propyl)- 4-hydroxypyrrolidine-2- carboxamide | 624.6 | 625.4 |
| 713 | 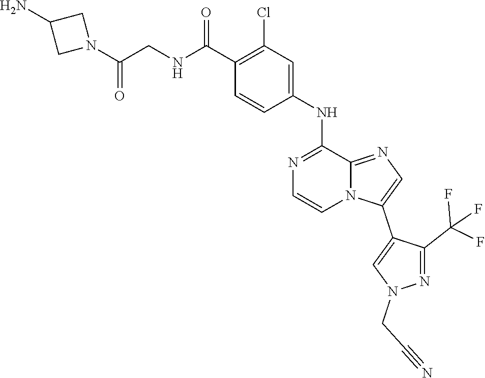 [SMILES: N#CCn1cc(-c2cnc3c(Nc4ccc(C(=O)NCC(=O)N5CC(N)C5)c(Cl)c4)nccn23)c(C(F)(F)F)n1] | N-[2-(3-aminoazetidin- 1-yl)-2-oxo- ethyl]-2-chloro-4-[[3-[1- (cyanomethyl)-3- (trifluoromethyl)pyrazol-4- yl]imidazo[1,2-a]pyrazin-8- yl]amino]benzamide formate | 572.9 | 573.1 |
| 714 | 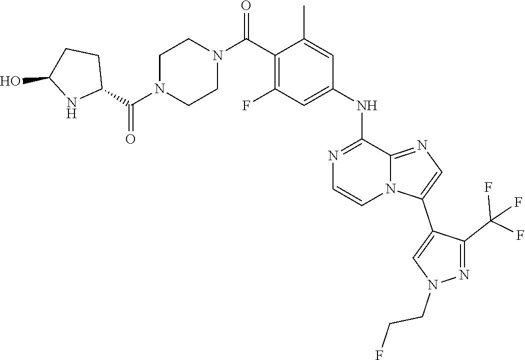 [SMILES: Cc1cc(Nc2nccn3c(-c4cn(CCF)nc4C(F)(F)F)cnc23)cc(F)c1C(=O)N1CCN(C(=O)[C@H]2CC[C@H](O)N2)CC1] | (4-(2-fluoro-4-((3-(1-(2- fluoroethyl)-3- (trifluoromethyl)- 1H-pyrazol-4-yl) imidazo[1,2- a]pyrazin-8-yl)amino)-6- methylbenzoyl)piperazin- 1-yl)((2R,5S)-5- hydroxypyrrolidin-2- yl)methanone | 647.6 | 648.4 |
| 715 | 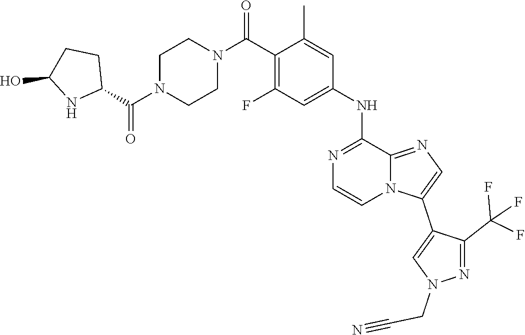 [SMILES: Cc1cc(Nc2nccn3c(-c4cn(CC#N)nc4C(F)(F)F)cnc23)cc(F)c1C(=O)N1CCN(C(=O)[C@H]2CC[C@H](O)N2)CC1] | 2-(4-(8-((3-fluoro-4- (4-((2R,5S)-5- hydroxypyrrolidine-2- carbonyl)piperazine- 1-carbonyl)-5- methylphenyl)amino) imidazo[1,2- a]pyrazin-3-yl)-3- (trifluoromethyl)- 1H-pyrazol-1-yl) acetonitrile | 640.6 | 641.3 |
| 716 | 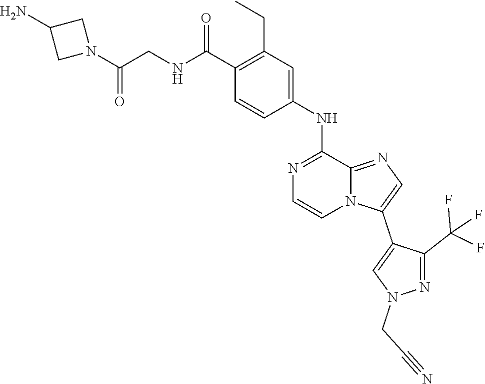 [SMILES: CCc1cc(Nc2nccn3c(-c4cn(CC#N)nc4C(F)(F)F)cnc23)ccc1C(=O)NCC(=O)N1CC(N)C1] | N-[2-(3-aminoazetidin- 1-yl)-2-oxo- ethyl]-4-[[3-[1- (cyanomethyl)-3- (trifluoromethyl)pyrazol-4- yl]imidazo[1,2-a]pyrazin- 8-yl]amino]-2-ethyl- benzamide formate | 566.5 | 567.2 |
| 717 | 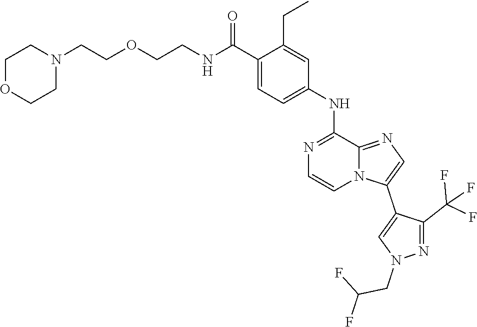 [SMILES: CCc1cc(Nc2nccn3c(-c4cn(CC(F)F)nc4C(F)(F)F)cnc23)ccc1C(=O)NCCOCCN1CCOCC1] | 4-((3-(1-(2,2- difluoroethyl)-3- (trifluoromethyl)- 1H-pyrazol-4- yl)imidazo[1,2-a] pyrazin-8- yl)amino)-2-ethyl- N-(2-(2- morpholinoethoxy) ethyl)benzamide formate | 636.6 | 637.4 |
| 718 | 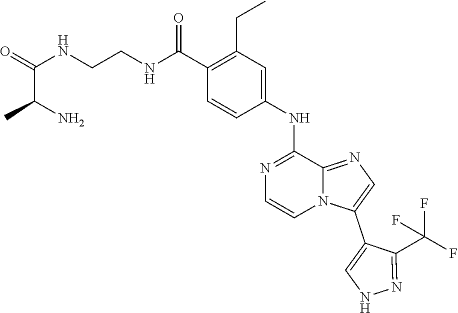 [SMILES: CCc1cc(Nc2nccn3c(-c4c[nH]nc4C(F)(F)F)cnc23)ccc1C(=O)NCCNC(=O)[C@H](C)N] | (S)-N-(2-(2- aminopropanamido) ethyl)-2-ethyl- 4-((3-(3-(trifluoromethyl)- 1H-pyrazol-4-yl)imidazo [1,2-a]pyrazin- 8-yl)amino)benzamide formate | 529.5 | 530.3 |
| 719 | 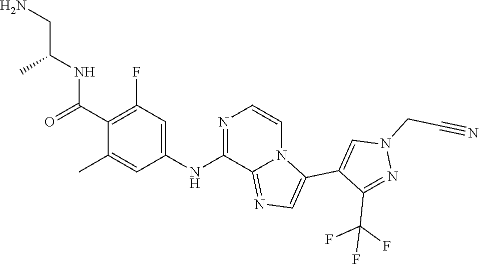 [SMILES: Cc1cc(Nc2nccn3c(-c4cn(CC#N)nc4C(F)(F)F)cnc23)cc(F)c1C(=O)N[C@H](C)CN] | (R)-N-(1-aminopropan- 2-yl)-4-((3- (1-(cyanomethyl)-3- (trifluoromethyl)- 1H-pyrazol-4- yl)imidazo[1,2-a]pyrazin- 8-yl)amino)-2-fluoro-6- methylbenzamide | 515.5 | 516.3 |
| 720 | 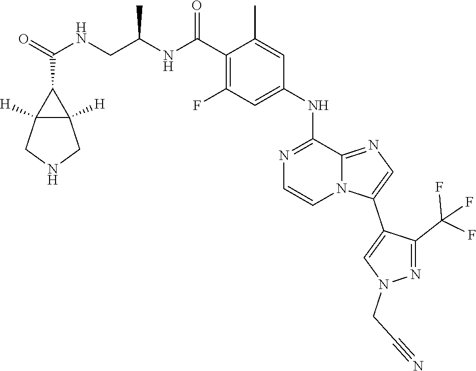 [SMILES: Cc1cc(Nc2nccn3c(-c4cn(CC#N)nc4C(F)(F)F)cnc23)cc(F)c1C(=O)N[C@H](C)CNC(=O)[C@H]1[C@@H]2CNC[C@@H]21] | (1R,5S,6r)-N-((R)-2- (4-((3-(1- (cyanomethyl)-3- (trifluoromethyl)- 1H-pyrazol-4-yl) imidazo[1,2- a]pyrazin-8-yl)amino)- 2-fluoro-6- methylbenzamido) propyl)-3- azabicyclo[3.1.0] hexane-6- carboxamide formate | 624.6 | 625.4 |
| 721 | 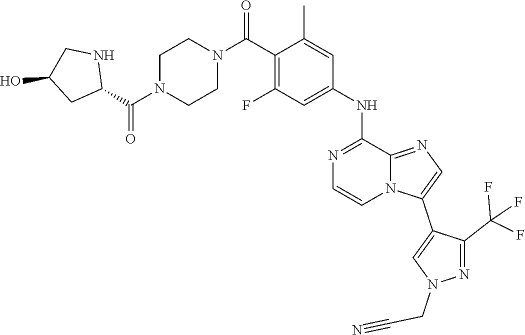 [SMILES: Cc1cc(Nc2nccn3c(-c4cn(CC#N)nc4C(F)(F)F)cnc23)cc(F)c1C(=O)N1CCN(C(=O)[C@@H]2C[C@@H](O)CN2)CC1] | 2-(4-(8-((3-fluoro-4- (4-((2S,4R)-4- hydroxypyrrolidine-2- carbonyl)piperazine-1- carbonyl)-5- methylphenyl)amino) imidazo[1,2- a]pyrazin-3-yl)-3- (trifluoromethyl)- 1H-pyrazol-1-yl) acetonitrile formate | 640.6 | 641.4 |
| 722 | 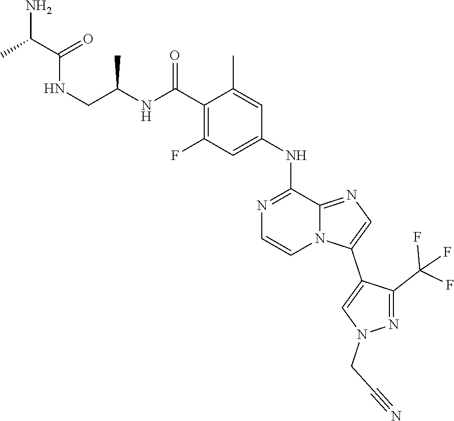 [SMILES: Cc1cc(Nc2nccn3c(-c4cn(CC#N)nc4C(F)(F)F)cnc23)cc(F)c1C(=O)N[C@H](C)CNC(=O)[C@H](C)N] | N-((R)-1-((S)-2- aminopropanamido) propan-2-yl)-4- ((3-(1-(cyanomethyl)-3- (trifluoromethyl)- 1H-pyrazol-4- yl)imidazo[1,2-a]pyrazin- 8-yl)amino)-2-fluoro-6- methylbenzamide formate | 586.6 | 587.3 |
| 723 | 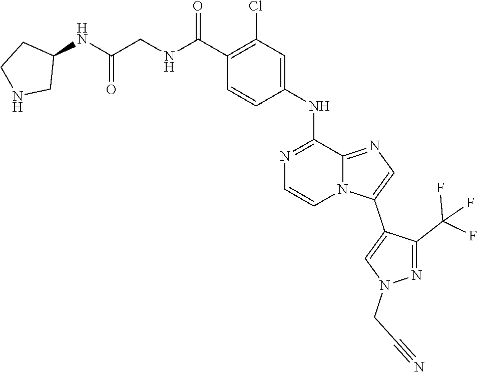 [SMILES: N#CCn1cc(-c2cnc3c(Nc4ccc(C(=O)NCC(=O)N[C@@H]5CCNC5)c(Cl)c4)nccn23)c(C(F)(F)F)n1] | 2-chloro-4-[[3-[1- (cyanomethyl)-3- (trifluoromethyl)pyrazol-4- yl]imidazo[1,2-a]pyrazin- 8-yl]amino]-N-[2- oxo-2-[[(3R)- pyrrolidin-3- yl]amino]ethyl]benzamide formate | 587.0 | 587.1 |
| 724 | 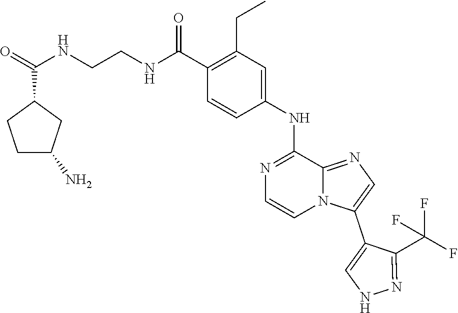 [SMILES: CCc1cc(Nc2nccn3c(-c4c[nH]nc4C(F)(F)F)cnc23)ccc1C(=O)NCCNC(=O)[C@H]1CC[C@@H](N)C1] | N-(2-((1S,3R)-3- aminocyclopentane-1- carboxamido)ethyl)- 2-ethyl-4-((3- (3-(trifluoromethyl)- 1H-pyrazol-4- yl)imidazo[1,2-a]pyrazin-8- yl)amino)benzamide | 569.6 | 570.3 |
| 725 | 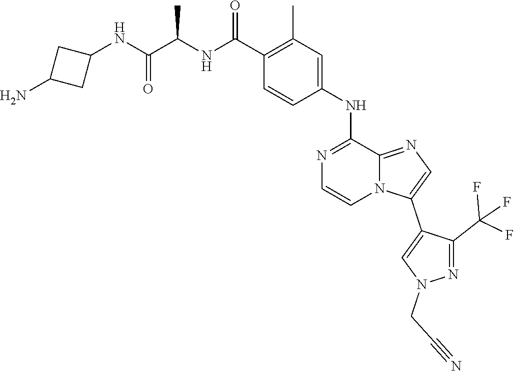 [SMILES: Cc1cc(Nc2nccn3c(-c4cn(CC#N)nc4C(F)(F)F)cnc23)ccc1C(=O)N[C@H](C)C(=O)NC1CC(N)C1] | N-[(1R)-2[(3- aminocyclobutyl)amino]- 1-methyl- 2-oxo-ethyl]-4-[[3-[1- (cyanomethyl)-3- (trifluoromethyl)pyrazol-4- yl]imidazo[1,2-a]pyrazin-8- yl]amino]-2-methyl- benzamide formate | 580.6 | 581.3 |
| 726 | 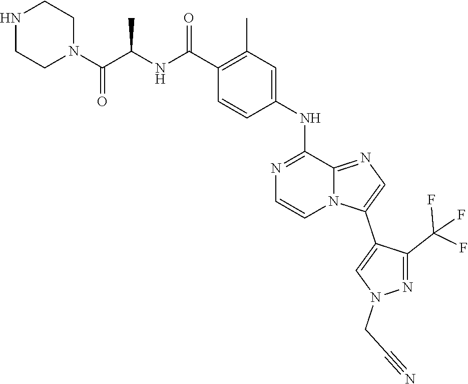 [SMILES: Cc1cc(Nc2nccn3c(-c4cn(CC#N)nc4C(F)(F)F)cnc23)ccc1C(=O)N[C@H](C)C(=O)N1CCNCC1] | 4-[[3-[1-(cyanomethyl)-3- (trifluoromethyl)pyrazol-4- yl]imidazo[1,2-a]pyrazin-8- yl]amino]-2-methyl- N-[(1R)-1- methyl-2-oxo-2- piperazin-1-yl- ethyl]benzamide formate | 580.6 | 581.3 |
| 727 | 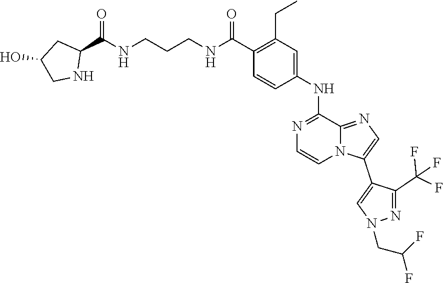 [SMILES: CCc1cc(Nc2nccn3c(-c4cn(CC(F)F)nc4C(F)(F)F)cnc23)ccc1C(=O)NCCCNC(=O)[C@@H]1C[C@@H](O)CN1] | (2S,4R)-N-(3-(4- ((3-(1-(2,2- difluoroethyl)-3- (trifluoromethyl)- 1H-pyrazol-4-yl) imidazo[1,2- a]pyrazin-8-yl)amino)-2- ethylbenzamido)propyl)-4- hydroxypyrrolidine-2- carboxamide | 649.6 | 648.5 [M − H]−, ESI neg |
| 729 | 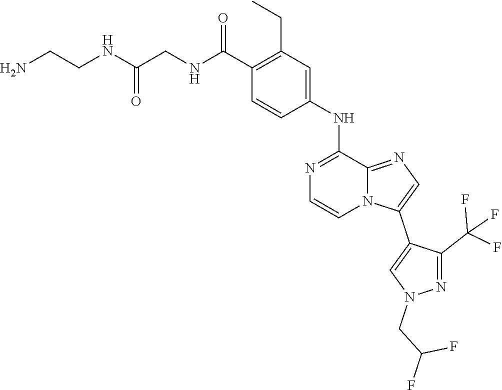 [SMILES: CCc1cc(Nc2nccn3c(-c4cn(CC(F)F)nc4C(F)(F)F)cnc23)ccc1C(=O)NCC(=O)NCCN] | N-[2-(2-aminoethylamino)- 2-oxo- ethyl]-4-[[3-[1-(2,2- difluoroethyl)- 3-(trifluoromethyl) pyrazol-4- yl]imidazo[1,2-a]pyrazin-8- yl]amino]-2-ethyl- benzamide | 579.5 | 580.2 |
| 730 | 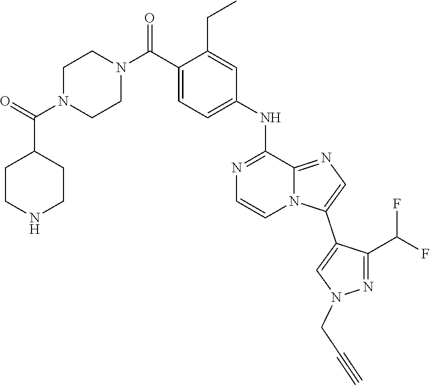 [SMILES: C#CCn1cc(-c2cnc3c(Nc4ccc(C(=O)N5CCN(C(=O)C6CCNCC6)CC5)c(CC)c4)nccn23)c(C(F)F)n1] | (4-(4-((3-(3- (difluoromethyl)-1- (prop-2-yn-1-yl)- 1H-pyrazol-4- yl)imidazo[1,2-a] pyrazin-8- yl)amino)-2- ethylbenzoyl)piperazin-1- yl)(piperidin-4-yl) methanone | 615.7 | 616.4 |
| 731 | 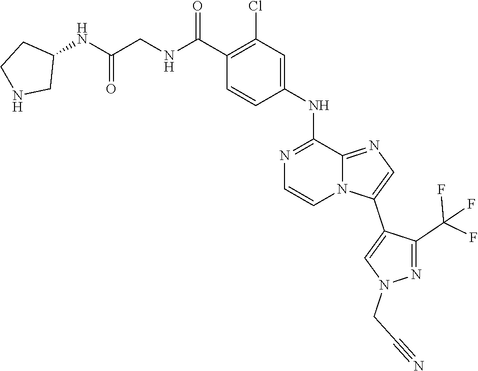 [SMILES: N#CCn1cc(-c2cnc3c(Nc4ccc(C(=O)NCC(=O)N[C@H]5CCNC5)c(Cl)c4)nccn23)c(C(F)(F)F)n1] | 2-chloro-4-[[3-[1- (cyanomethyl)-3- (trifluoromethyl)pyrazol-4- yl]imidazo[1,2-a]pyrazin-8- yl]amino]-N-[2-oxo-2-[[(3S)- pyrrolidin-3- yl]amino]ethyl]benzamide formate | 587.0 | 587.1 |
| 732 | 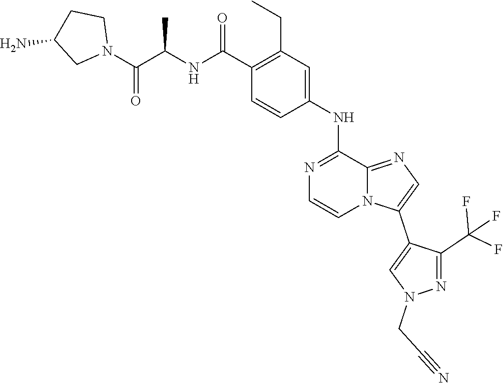 [SMILES: CCc1cc(Nc2nccn3c(-c4cn(CC#N)nc4C(F)(F)F)cnc23)ccc1C(=O)N[C@H](C)C(=O)N1CC[C@@H](N)C1] | N-[(1R)-2-[(3R)-3- aminopyrrolidin- 1-yl]-1-methyl-2-oxo- ethyl]-4-[[3- [1-(cyanomethyl)-3- (trifluoromethyl)pyrazol-4- yl]imidazo[1,2-a] pyrazin-8- yl]amino]-2-ethyl- benzamide formate | 594.6 | 595.2 |
| 733 | 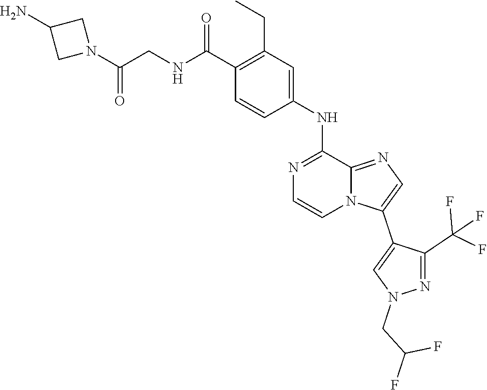 [SMILES: CCc1cc(Nc2nccn3c(-c4cn(CC(F)F)nc4C(F)(F)F)cnc23)ccc1C(=O)NCC(=O)N1CC(N)C1] | N-[2-(3-aminoazetidin- 1-yl)-2-oxo- ethyl]-4-[[3-[1-(2,2- difluoroethyl)- 3-(trifluoromethyl)pyrazol-4- yl]imidazo[1,2-a] pyrazin-8- yl]amino]-2-ethyl- benzamide | 591.5 | 592.1 |
| 734 | 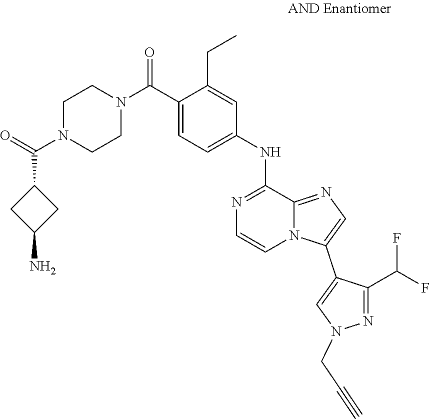 [SMILES: C#CCn1cc(-c2cnc3c(Nc4ccc(C(=O)N5CCN(C(=O)[C@H]6C[C@H](N)C6)CC5)c(CC)c4)nccn23)c(C(F)F)n1] | (4-((1r,3r)-3- aminocyclobutane-1- earbonyl)piperazin-1- yl)(4-((3-(3- (difluoromethyl)-1- (prop-2-yn-1- yl)-1H-pyrazol-4- yl)imidazo[1,2- a]pyrazin-8-yl)amino)-2- ethylphenyl)methanone | 601.7 | 602.4 |
| 735 | 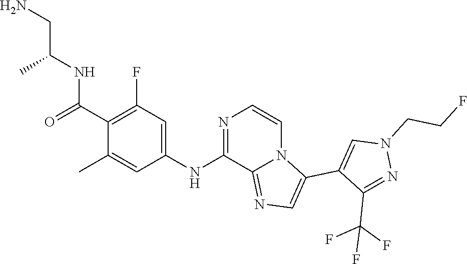 [SMILES: Cc1cc(Nc2nccn3c(-c4cn(CCF)nc4C(F)(F)F)cnc23)cc(F)c1C(=O)N[C@H](C)CN] | (R)-N-(1-aminopropan- 2-yl)-2- fluoro-4-((3-(1-(2- fluoroethyl)-3- (trifluoromethyl)- 1H-pyrazol-4- yl)imidazo[1,2-a] pyrazin-8- yl)amino)-6- methylbenzamide | 522.5 | 523.3 |
| 736 | 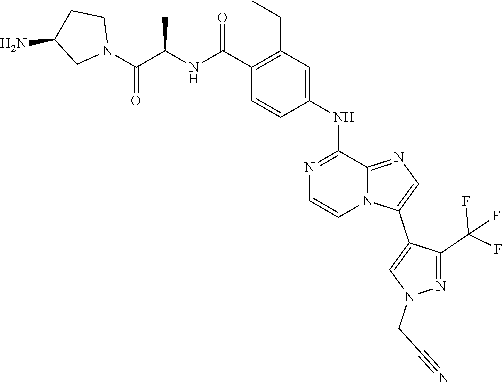 [SMILES: CCc1cc(Nc2nccn3c(-c4cn(CC#N)nc4C(F)(F)F)cnc23)ccc1C(=O)N[C@H](C)C(=O)N1CC[C@H](N)C1] | N-[(1R)-2-[(3S)-3- aminopyrrolidin- 1-yl]-1-methyl-2- oxo-ethyl]-4-[[3- [1-(cyanomethyl)-3- (trifluoromethyl)pyrazol-4- yl]imidazo[1,2-a] pyrazin-8- yl]amino]-2-ethyl- benzamide formate | 594.6 | 595.5 |
| 737 | 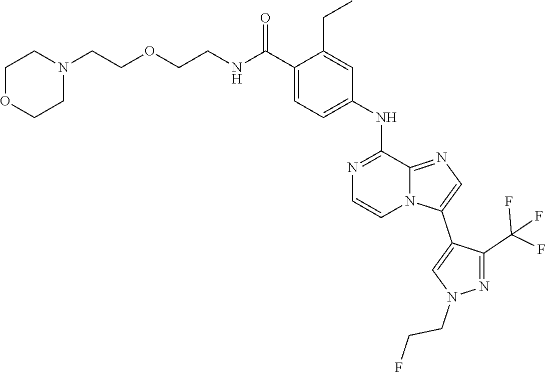 [SMILES: CCc1cc(Nc2nccn3c(-c4cn(CCF)nc4C(F)(F)F)cnc23)ccc1C(=O)NCCOCCN1CCOCC1] | 2-ethyl-4-((3-(1-(2- fluoroethyl)-3- (trifluoromethyl)- 1H-pyrazol-4- yl)imidazo[1,2-a] pyrazin-8- yl)amino)-N-(2-(2- morpholinoethoxy) ethyl)benzamide formate | 618.6 | 619.4 |
| 738 | 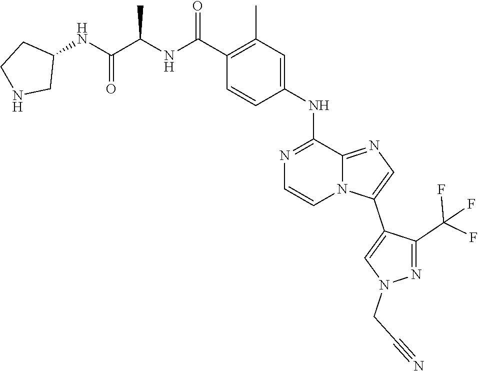 [SMILES: Cc1cc(Nc2nccn3c(-c4cn(CC#N)nc4C(F)(F)F)cnc23)ccc1C(=O)N[C@H](C)C(=O)N[C@H]1CCNC1] | 4-[[3-[1-(cyanomethyl)-3- (trifluoromethyl)pyrazol-4- yl]imidazo[1,2-a]pyrazin-8- yl]amino]-2-methyl- N-[(1R)-1- methyl-2-oxo-2-[[(3S)- pyrrolidin-3- yl]amino]ethyl]benzamide formate | 580.6 | 581.3 |
| 739 | 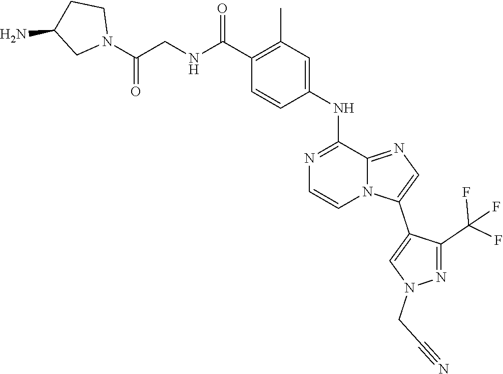 [SMILES: Cc1cc(Nc2nccn3c(-c4cn(CC#N)nc4C(F)(F)F)cnc23)ccc1C(=O)NCC(=O)N1CC[C@H](N)C1] | N-[2-[(3S)-3- aminopyrrolidin-1-yl]- 2-oxo-ethyl]-4-[[3-[1- (cyanomethyl)-3- (trifluoromethyl)pyrazol-4- yl]imidazo[1,2-a]pyrazin-8- yl]amino]-2-methyl- benzamide formate | 566.5 | 567.3 |
| 740 | 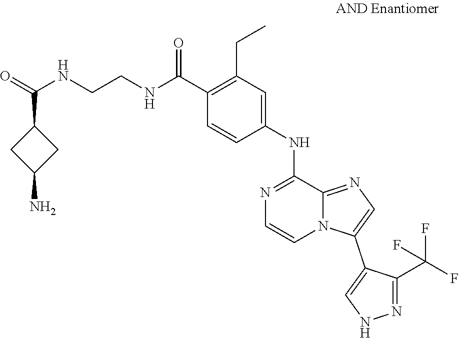 [SMILES: CCc1cc(Nc2nccn3c(-c4c[nH]nc4C(F)(F)F)cnc23)ccc1C(=O)NCCNC(=O)[C@H]1C[C@@H](N)C1] | N-(2-((1s,3s)-3- aminocyclobutane- 1-carboxamido)ethyl)- 2-ethyl-4-((3- (3-(trifluoromethyl)- 1H-pyrazol-4- yl)imidazo[1,2-a]pyrazin-8- yl)amino)benzamide | 555.6 | 556.3 |
| 741 | 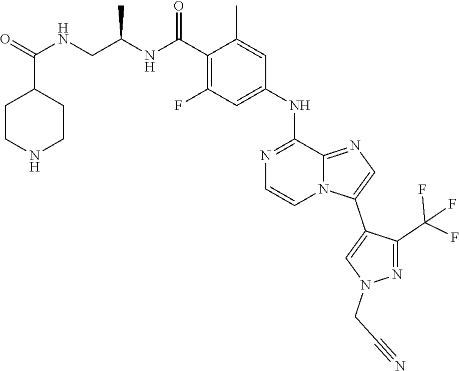 [SMILES: Cc1cc(Nc2nccn3c(-c4cn(CC#N)nc4C(F)(F)F)cnc23)cc(F)c1C(=O)N[C@H](C)CNC(=O)C1CCNCC1] | (R)-N-(2-(4-((3-(1- (cyanomethyl)- 3-(trifluoromethyl)- 1H-pyrazol-4- yl)imidazo[1,2-a]pyrazin-8- yl)amino)-2-fluoro-6- methylbenzamido) propyl)piperidine- 4-carboxamide formate | 626.6 | 627.4 |
| 742 | 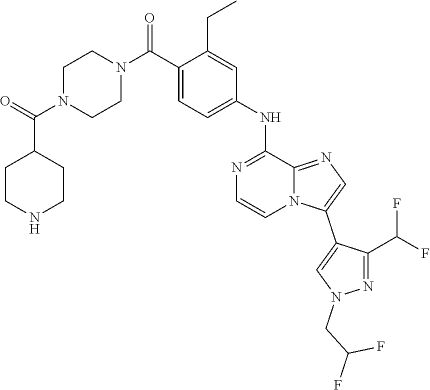 [SMILES: CCc1cc(Nc2nccn3c(-c4cn(CC(F)F)nc4C(F)F)cnc23)ccc1C(=O)N1CCN(C(=O)C2CCNCC2)CC1] | (4-(4-((3-(1-(2,2- difluoroethyl)-3- (difluoromethyl)- 1H-pyrazol-4- yl)imidazo[1,2-a] pyrazin-8- yl)amino)-2- ethylbenzoyl)piperazin-1- yl)(piperidin-4-yl) methanone | 641.7 | 642.4 |
| 743 | 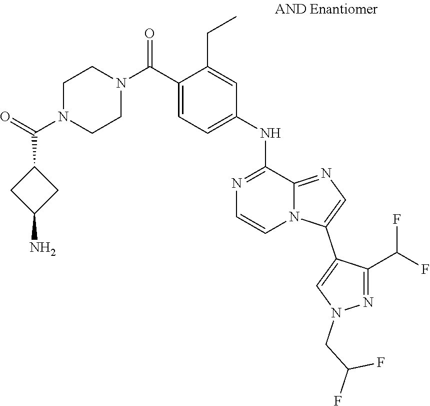 [SMILES: CCc1cc(Nc2nccn3c(-c4cn(CC(F)F)nc4C(F)F)cnc23)ccc1C(=O)N1CCN(C(=O)[C@H]2C[C@H](N)C2)CC1] | (4-((1r,3r)-3- aminocyclobutane-1- carbonyl)piperazin- 1-yl)(4-((3-(1- (2,2-difluoroethyl)-3- (difluoromethyl)- 1H-pyrazol-4- yl)imidazo[1,2-a] pyrazin-8- yl)amino)-2-ethylphenyl) methanone | 627.6 | 628.5 |
| 744 | 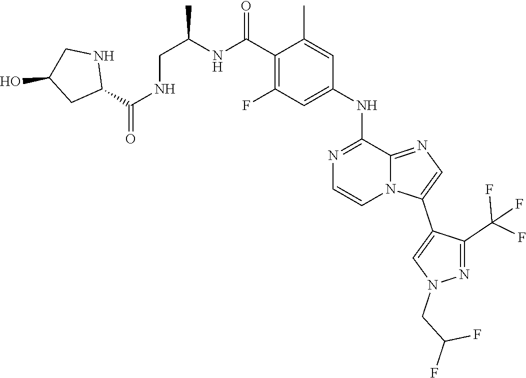 [SMILES: Cc1cc(Nc2nccn3c(-c4cn(CC(F)F)nc4C(F)(F)F)cnc23)cc(F)c1C(=O)N[C@H](C)CNC(=O)[C@@H]1C[C@@H](O)CN1] | (2S,4R)-N-((R)- 2-(4-((3-(1-(2,2- difluoroethyl)-3- (trifluoromethyl)- 1H-pyrazol-4-yl) imidazo[1,2- a]pyrazin-8-yl) amino)-2-fluoro-6- methylbenzamido)propyl)- 4-hydroxypyrrolidine-2- carboxamide | 653.6 | 654.4 |
| 745 | 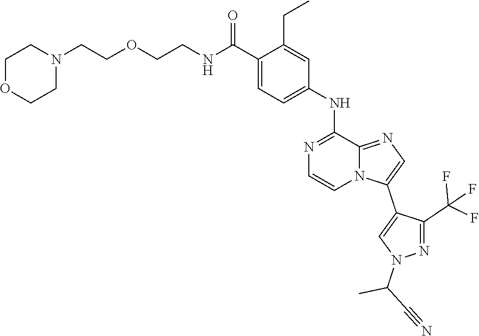 [SMILES: CCc1cc(Nc2nccn3c(-c4cn(C(C)C#N)nc4C(F)(F)F)cnc23)ccc1C(=O)NCCOCCN1CCOCC1] | 4-((3-(1-(1-cyanoethyl)-3- (trifluoromethyl)- 1H-pyrazol-4- yl)imidazo[1,2-a]pyrazin-8- yl)amino)-2-ethyl-N-(2-(2- morpholinoethoxy)ethyl) benzamide formate | 625.7 | 626.4 |
| 746 | 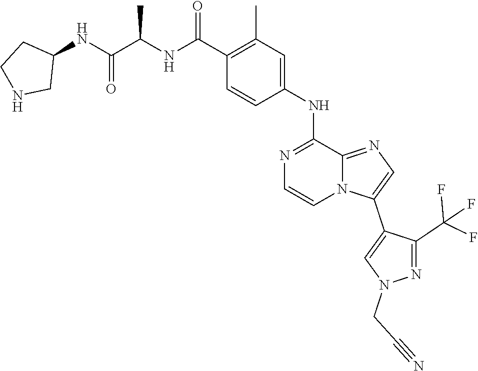 [SMILES: Cc1cc(Nc2nccn3c(-c4cn(CC#N)nc4C(F)(F)F)cnc23)ccc1C(=O)N[C@H](C)C(=O)N[C@@H]1CCNC1] | 4-[[3-[1-(cyanomethyl)-3- (trifluoromethyl)pyrazol-4- yl]imidazo[1,2-a]pyrazin-8- yl]amino]-2-methyl- N-[(1R)-1- methyl-2-oxo-2- [[(3R)-pyrrolidin- 3-yl]amino]ethyl] benzamide formate | 580.6 | 581.3 |
| 747 | 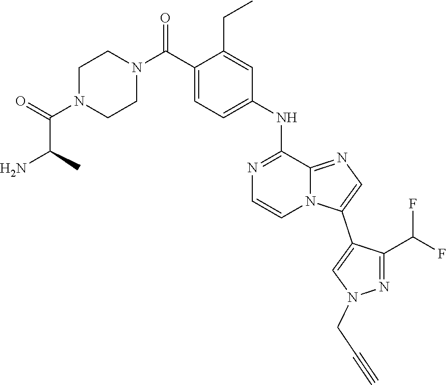 [SMILES: C#CCn1cc(-c2cnc3c(Nc4ccc(C(=O)N5CCN(C(=O)[C@@H](C)N)CC5)c(CC)c4)nccn23)c(C(F)F)n1] | (R)-2-amino-1-(4-(4-((3-(3- (difluoromethyl)-1- (prop-2-yn-1- yl)-1H-pyrazol-4-yl) imidazo[1,2- a]pyrazin-8-yl)amino)-2- ethylbenzoyl)piperazin-1- yl)propan-1-one | 575.6 | 576.4 |
| 748 | 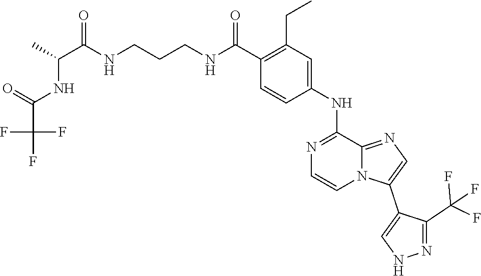 [SMILES: CCc1cc(Nc2nccn3c(-c4c[nH]nc4C(F)(F)F)cnc23)ccc1C(=O)NCCCNC(=O)[C@@H](C)NC(=O)C(F)(F)F] | (R)-2-ethyl-N-(3-(2-(2,2,2- trifluoroacetamido) propanamido) propyl)-4-((3-(3- (trifluoromethyl)- 1H-pyrazol-4- yl)imidazo[1,2- a]pyrazin-8-yl)amino) benzamide | 639.6 | 638.5 [M − H]−, ESI neg |
| 749 | 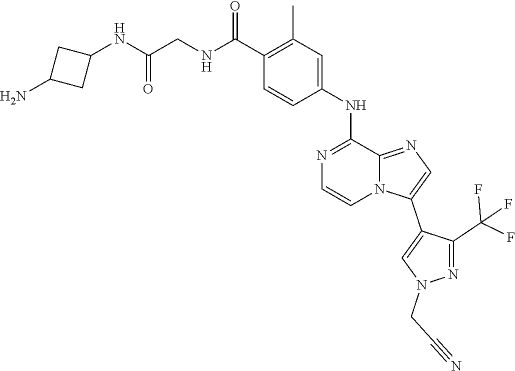 [SMILES: Cc1cc(Nc2nccn3c(-c4cn(CC#N)nc4C(F)(F)F)cnc23)ccc1C(=O)NCC(=O)NC1CC(N)C1] | N-[2-[(3-aminocyclobutyl) amino]- 2-oxo-ethyl]-4-[[3-[1- (cyanomethyl)-3- (trifluoromethyl)pyrazol-4- yl]imidazo[1,2-a]pyrazin-8- yl]amino]-2-methyl- benzamide formate | 566.5 | 567.2 |
| 750 | 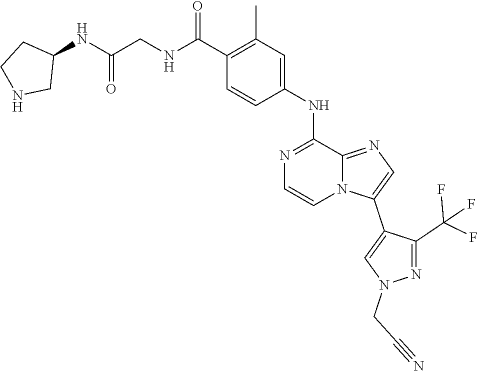 [SMILES: Cc1cc(Nc2nccn3c(-c4cn(CC#N)nc4C(F)(F)F)cnc23)ccc1C(=O)NCC(=O)N[C@@H]1CCNC1] | 4-[[3-[1-(cyanomethyl)-3- (trifluoromethyl)pyrazol-4- yl]imidazo[1,2-a]pyrazin-8- yl]amino]-2-methyl- N-[2-oxo-2- [[(3R)-pyrrolidin-3- yl]amino]ethyl]benzamide formate | 566.5 | 567.3 |
| 751 | 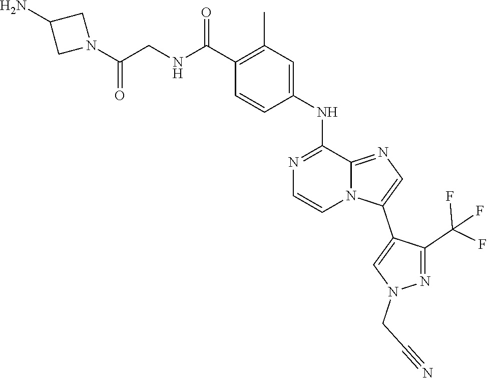 [SMILES: Cc1cc(Nc2nccn3c(-c4cn(CC#N)nc4C(F)(F)F)cnc23)ccc1C(=O)NCC(=O)N1CC(N)C1] | N-[2-(3-aminoazetidin- 1-yl)-2-oxo- ethyl]-4-[[3-[1- (cyanomethyl)-3- (trifluoromethyl)pyrazol-4- yl]imidazo[1,2-a]pyrazin-8- yl]amino]-2-methyl- benzamide formate | 552.5 | 553.3 |
| 752 | 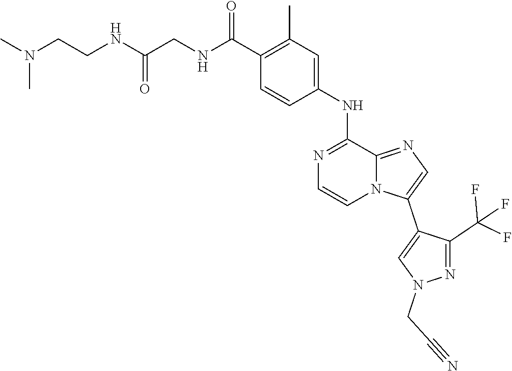 [SMILES: Cc1cc(Nc2nccn3c(-c4cn(CC#N)nc4C(F)(F)F)cnc23)ccc1C(=O)NCC(=O)NCCN(C)C] | 4-[[3-[1-(cyanomethyl)-3- (trifluoromethyl)pyrazol-4- yl]imidazo[1,2-a]pyrazin-8- yl]amino]-N-[2-[2- (dimethylamino) ethylamino]-2-oxo- ethyl]-2-methyl- benzamide formate | 568.6 | 569.3 |
| 753 | 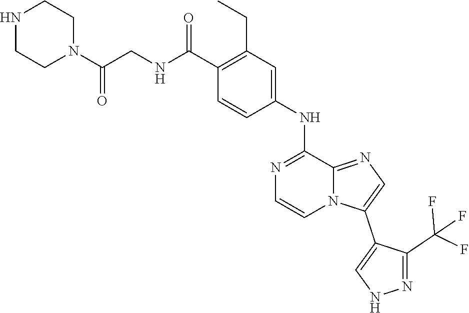 [SMILES: CCc1cc(Nc2nccn3c(-c4c[nH]nc4C(F)(F)F)cnc23)ccc1C(=O)NCC(=O)N1CCNCC1] | 2-ethyl-N-(2-oxo- 2-piperazin-1-yl- ethyl)-4-[[3-[3- (trifluoromethyl)- 1H-pyrazol-4-yl] imidazo[1,2- a]pyrazin-8-yl]amino] benzamide formate | 541.5 | 542.3 |
| 754 | 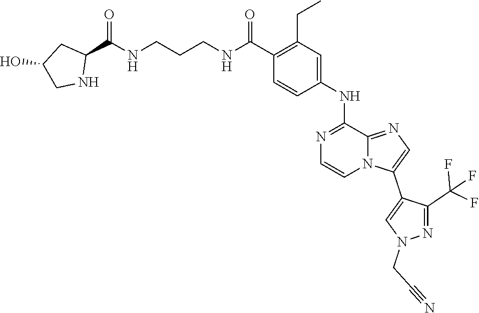 [SMILES: CCc1cc(Nc2nccn3c(-c4cn(CC#N)nc4C(F)(F)F)cnc23)ccc1C(=O)NCCCNC(=O)[C@@H]1C[C@@H](O)CN1] | (2S,4R)-N-(3-(4-((3-(1- (cyanomethyl)-3- (trifluoromethyl)-1H- pyrazol-4-yl)imidazo[1,2- a]pyrazin-8-yl)amino)-2- ethylbenzamido)propyl)-4- hydroxypyrrolidine-2- carboxamide | 624.6 | 625.3 |
| 755 | 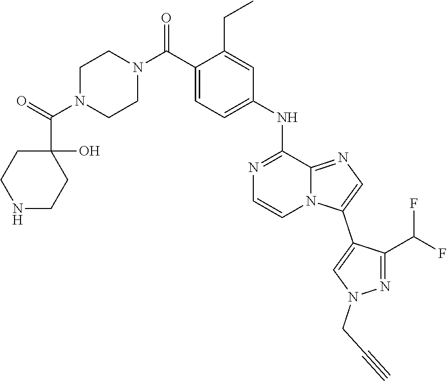 [SMILES: C#CCn1cc(-c2cnc3c(Nc4ccc(C(=O)N5CCN(C(=O)C6(O)CCNCC6)CC5)c(CC)c4)nccn23)c(C(F)F)n1] | (4-(4-((3-(3- (difluoromethyl)-1- (prop-2-yn-1-yl)- 1H-pyrazol-4- yl)imidazo[1,2-a] pyrazin-8- yl)amino)-2- ethylbenzoyl) piperazin-1-yl)(4- hydroxypiperidin-4-yl) methanone | 631.7 | 632.4 |
| 756 | 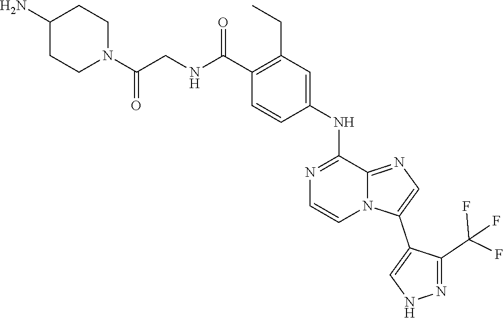 [SMILES: CCc1cc(Nc2nccn3c(-c4c[nH]nc4C(F)(F)F)cnc23)ccc1C(=O)NCC(=O)N1CCC(N)CC1] | N-[2-(4-amino-1- piperidyl)-2-oxo- ethyl]-2-ethyl-4-[[3-[3- (trifluoromethyl)- 1H-pyrazol-4- yl]imidazo[1,2-a]pyrazin-8- yl]amino]benzamide formate | 555.6 | 556.3 |
| 757 | 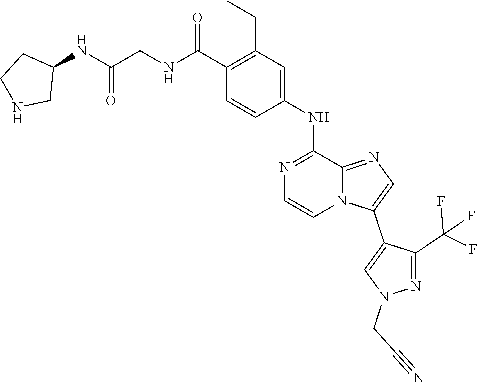 [SMILES: CCc1cc(Nc2nccn3c(-c4cn(CC#N)nc4C(F)(F)F)cnc23)ccc1C(=O)NCC(=O)N[C@@H]1CCNC1] | 4-[[3-[1-(cyanomethyl)-3- (trifluoromethyl)pyrazol-4- yl]imidazo[1,2-a]pyrazin-8- yl]amino]-2-ethyl-N-[2-oxo-2- [[(3R)-pyrrolidin-3- yl]amino]ethyl]benzamide formate | 580.6 | 581.2 |
| 758 | 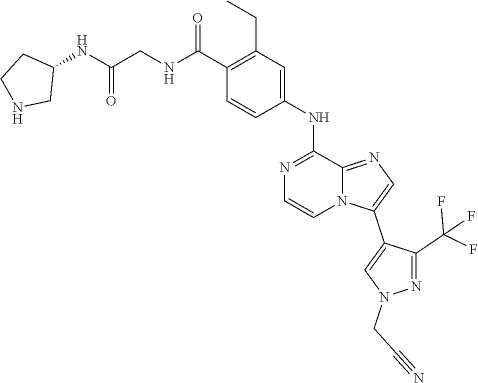 [SMILES: CCc1cc(Nc2nccn3c(-c4cn(CC#N)nc4C(F)(F)F)cnc23)ccc1C(=O)NCC(=O)N[C@H]1CCNC1] | 4-[[3-[1-(cyanomethyl)-3- (trifluoromethyl)pyrazol-4- yl]imidazo[1,2-a]pyrazin-8- yl]amino]-2-ethyl-N-[2-oxo-2- [[(3S)-pyrrolidin-3- yl]amino]ethyl]benzamide formate | 580.6 | 581.2 |
| 759 | 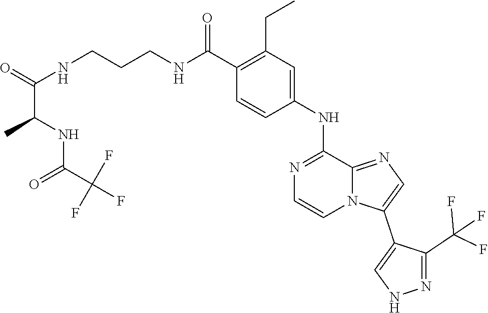 [SMILES: CCc1cc(Nc2nccn3c(-c4c[nH]nc4C(F)(F)F)cnc23)ccc1C(=O)NCCCNC(=O)[C@H](C)NC(=O)C(F)(F)F] | (S)-2-ethyl-N-(3-(2-(2,2,2- trifluoroacetamido) propanamido) propyl)-4-((3-(3- (trifluoromethyl)- 1H-pyrazol-4-yl) imidazo[1,2- a]pyrazin-8-yl)amino) benzamide | 639.6 | 640.4 |
| 760 | 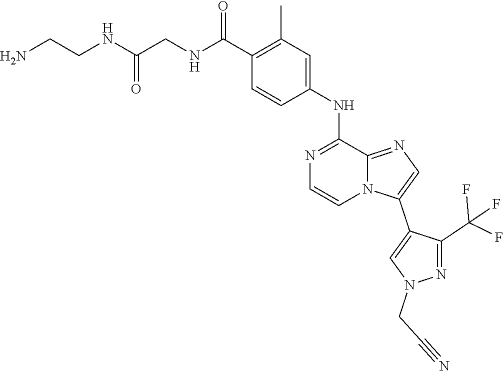 [SMILES: Cc1cc(Nc2nccn3c(-c4cn(CC#N)nc4C(F)(F)F)cnc23)ccc1C(=O)NCC(=O)NCCN] | N-[2-(2-aminoethylamino)- 2-oxo- ethyl]-4-[[3-[1- (cyanomethyl)-3- (trifluoromethyl)pyrazol-4- yl]imidazo[1,2-a]pyrazin-8- yl]amino]-2-methyl- benzamide formate | 540.5 | 541.2 |
| 761 | 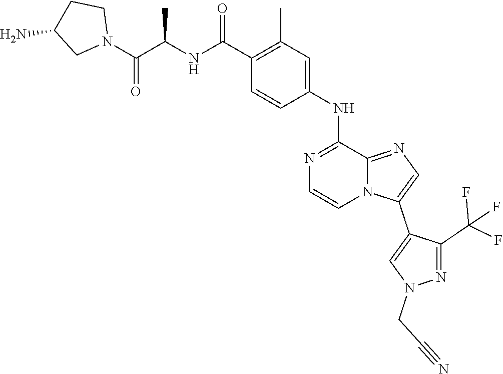 [SMILES: Cc1cc(Nc2nccn3c(-c4cn(CC#N)nc4C(F)(F)F)cnc23)ccc1C(=O)N[C@H](C)C(=O)N1CC[C@@H](N)C1] | N-[(1R)-2-[(3R)-3- aminopyrrolidin- 1-yl]-1-methyl-2-oxo- ethyl]-4-[[3- [1-(cyanomethyl)-3- (trifluoromethyl)pyrazol-4- yl]imidazo[1,2-a]pyrazin-8- yl]amino]-2-methyl-b enzamide formate | 580.6 | 581.2 |
| 762 | 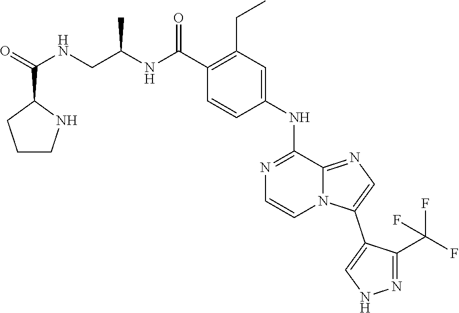 [SMILES: CCc1cc(Nc2nccn3c(-c4c[nH]nc4C(F)(F)F)cnc23)ccc1C(=O)N[C@H](C)CNC(=O)[C@@H]1CCCN1] | (S)-N-((R)-2-(2-ethyl- 4-((3-(3- (trifluoromethyl)- 1H-pyrazol-4- yl)imidazo[1,2-a]pyrazin-8- yl)amino)benzamido) propyl) pyrrolidine-2-carboxamide | 569.6 | 570.4 |
| 763 | 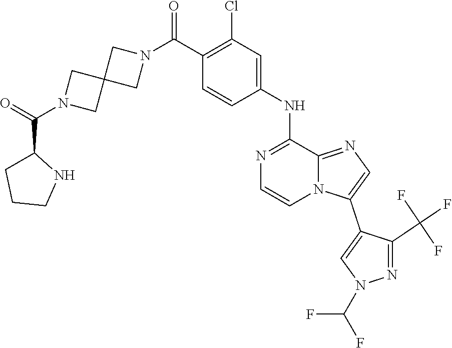 [SMILES: O=C(c1ccc(Nc2nccn3c(-c4cn(C(F)F)nc4C(F)(F)F)cnc23)cc1Cl)N1CC2(C1)CN(C(=O)[C@@H]1CCCN1)C2] | (S)-(2-chloro-4-((3-(1- (difluoromethyl)-3- (trifluoromethyl)- 1H-pyrazol-4- yl)imidazo[1,2-a]pyrazin-8- yl)amino)phenyl)(6- prolyl-2,6- diazaspiro[3.3]heptan-2- yl)methanone | 650.0 | 648.4 [M − H]−, ESI neg |
| 764 | 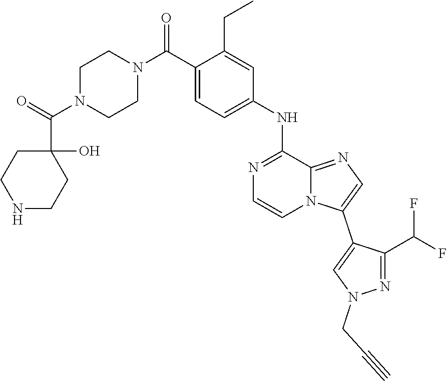 [SMILES: C#CCn1cc(-c2cnc3c(Nc4ccc(C(=O)N5CCN(C(=O)C6(O)CCNCC6)CC5)c(CC)c4)nccn23)c(C(F)F)n1] | (4-(4-((3-(1-(2,2- difluoroethyl)-3- (difluoromethyl)-1H-pyrazol-4- yl)imidazo[1,2-a]pyrazin-8- yl)amino)-2- ethylbenzoyl)piperazin- 1-yl)(4- hydroxypiperidin-4- yl)methanone | 657.7 | 658.4 |
| 765 | 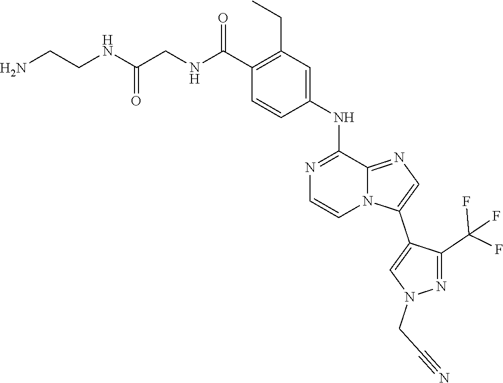 [SMILES: CCc1cc(Nc2nccn3c(-c4cn(CC#N)nc4C(F)(F)F)cnc23)ccc1C(=O)NCC(=O)NCCN] | N-[2-(2-aminoethylamino)- 2-oxo- ethyl]-4-[[3-[1- (cyanomethyl)-3- (trifluoromethyl)pyrazol-4- yl]imidazo[1,2-a]pyrazin-8- yl]amino]-2-ethyl-benzamide | 554.5 | 555.1 |
| 766 | 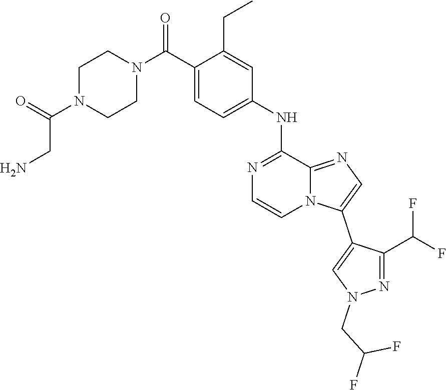 [SMILES: CCc1cc(Nc2nccn3c(-c4cn(CC(F)F)nc4C(F)F)cnc23)ccc1C(=O)N1CCN(C(=O)CN)CC1] | 2-amino-1-(4-(4-((3-(1-(2,2- difluoroethyl)-3- (difluoromethyl)- 1H-pyrazol-4-yl)imidazo[1,2- a]pyrazin-8-yl)amino)-2- ethylbenzoyl)piperazin- 1-yl)ethan- 1-one | 587.6 | 588.3 |
| 767 | 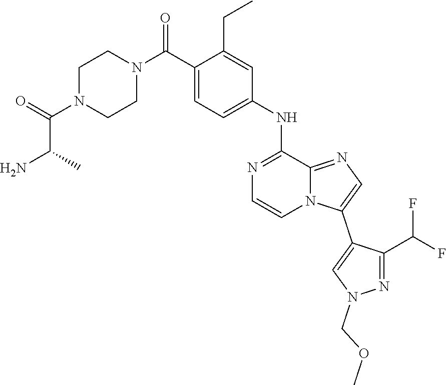 [SMILES: CCc1cc(Nc2nccn3c(-c4cn(COC)nc4C(F)F)cnc23)ccc1C(=O)N1CCN(C(=O)[C@H](C)N)CC1] | (S)-2-amino-1-(4-(4-((3-(3- (difluoromethyl)-1- (methoxymethyl)-1H- pyrazol-4- yl)imidazo[1,2-a]pyrazin-8- yl)amino)-2- ethylbenzoyl)piperazin-1- yl)propan-1-one | 581.6 | 582.3 |
| 768 | 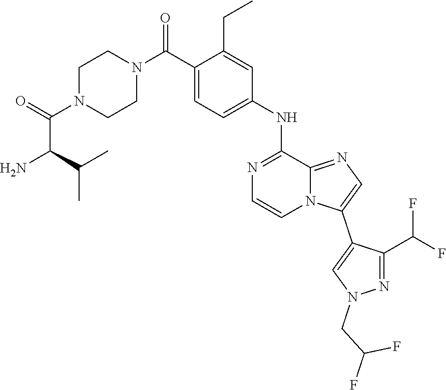 [SMILES: CCc1cc(Nc2nccn3c(-c4cn(CC(F)F)nc4C(F)F)cnc23)ccc1C(=O)N1CCN(C(=O)[C@H](N)C(C)C)CC1] | (R)-2-amino-1-(4- (4-((3-(1-(2,2- difluoroethyl)-3- (difluoromethyl)- 1H-pyrazol-4-yl)imidazo[1,2- a]pyrazin-8-yl)amino)-2- ethylbenzoyl)piperazin- 1-yl)-3- methylbutan-1-one | 629.7 | 630.5 |
| 769 | 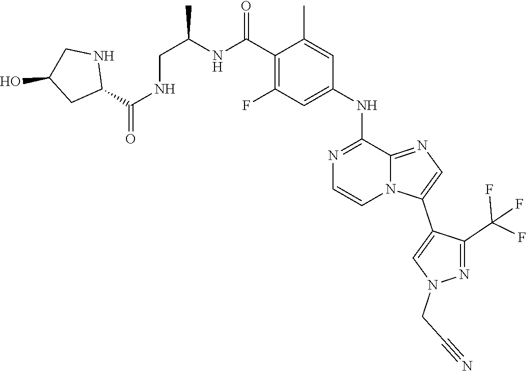 [SMILES: Cc1cc(Nc2nccn3c(-c4cn(CC#N)nc4C(F)(F)F)cnc23)cc(F)c1C(=O)N[C@H](C)CNC(=O)[C@@H]1C[C@@H](O)CN1] | (2S,4R)-N-((R)-2-(4-(3-(1- (cyanomethyl)-3- (trifluoromethyl)- 1H-pyrazol-4-yl) imidazo[1,2- a]pyrazin-8-yl)amino)- 2-fluoro-6- methylbenzamido)propyl)-4- hydroxypyrrolidine-2- carboxamide formate | 628.6 | 629.4 |
| 770 | 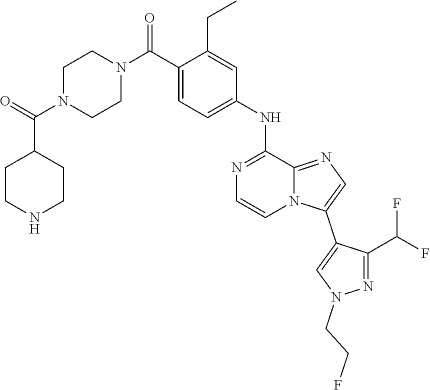 [SMILES: CCc1cc(Nc2nccn3c(-c4cn(CCF)nc4C(F)F)cnc23)ccc1C(=O)N1CCN(C(=O)C2CCNCC2)CC1] | (4-(4-((3-(3- (difluoromethyl)-1-(2- fluoroethyl)-1H-pyrazol-4- yl)imidazo[1,2-a]pyrazin-8- yl)amino)-2- ethylbenzoyl)piperazin-1- yl)(piperidin-4-yl)methanone formate | 623.7 | 624.4 |
| 771 | 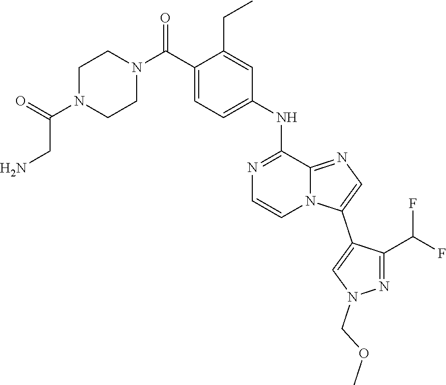 [SMILES: CCc1cc(Nc2nccn3c(-c4cn(COC)nc4C(F)F)cnc23)ccc1C(=O)N1CCN(C(=O)CN)CC1] | 2-amino-1-(4-(4-((3-(3- (difluoromethyl)-1- (methoxymethyl)- 1H-pyrazol-4- yl)imidazo[1,2-a]pyrazin-8- yl)amino)-2- ethylbenzoyl)piperazin- 1-yl)ethan- 1-one | 567.6 | 568.3 |
| 772 | 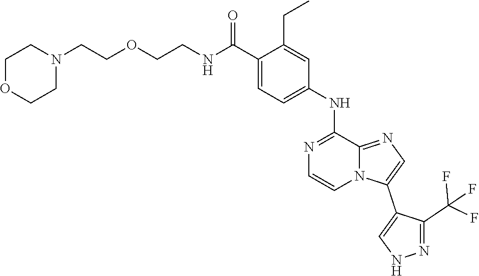 [SMILES: CCc1cc(Nc2nccn3c(-c4c[nH]nc4C(F)(F)F)cnc23)ccc1C(=O)NCCOCCN1CCOCC1] | 2-ethyl-N-(2-(2- morpholinoethoxy) ethyl)-4-((3-(3- (trifluoromethyl)- 1H-pyrazol-4- yl)imidazo[1,2-a]pyrazin-8- yl)amino)benzamide | 572.6 | 573.4 |
| 773 | 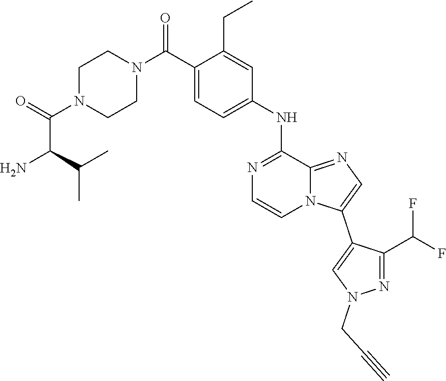 [SMILES: C#CCn1cc(-c2cnc3c(Nc4ccc(C(=O)N5CCN(C(=O)[C@H](N)C(C)C)CC5)c(CC)c4)nccn23)c(C(F)F)n1] | (R)-2-amino-1-(4-(4-((3-(3- (difluoromethyl)-1- (prop-2-yn-1- yl)-1H-pyrazol-4-yl) imidazo[1,2- a]pyrazin-8-yl)amino)-2- ethylbenzoyl)piperazin-1-yl)-3- methylbutan-1-one | 603.7 | 604.4 |
| 774 | 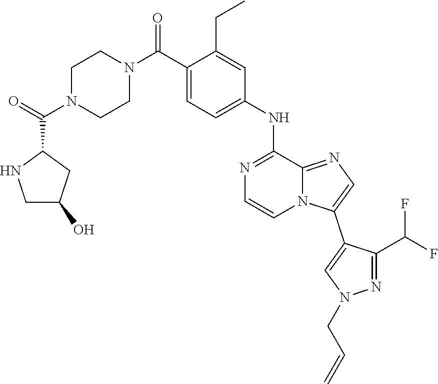 [SMILES: C=CCn1cc(-c2cnc3c(Nc4ccc(C(=O)N5CCN(C(=O)[C@@H]6C[C@@H](O)CN6)CC5)c(CC)c4)nccn23)c(C(F)F)n1] | (4-(4-((3-(1-allyl-3- (difluoromethyl)- 1H-pyrazol-4- yl)imidazo[1,2-a]pyrazin-8- yl)amino)-2- ethylbenzoyl)piperazin-1- yl)((2S,4R)-4- hydroxypyrrolidin-2- yl)methanone | 619.7 | 620.4 |
| 775 | 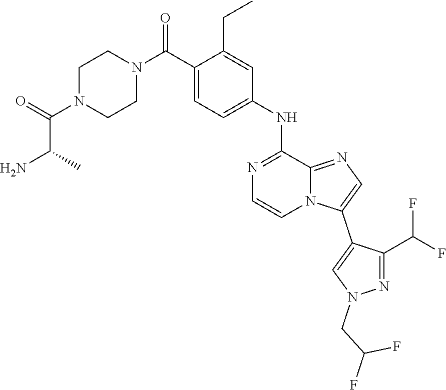 [SMILES: CCc1cc(Nc2nccn3c(-c4cn(CC(F)F)nc4C(F)F)cnc23)ccc1C(=O)N1CCN(C(=O)[C@H](C)N)CC1] | (S)-2-amino-1-(4- (4-((3-(1-(2,2- difluoroethyl)-3- (difluoromethyl)- 1H-pyrazol-4-yl) imidazo[1,2- a]pyrazin-8-yl)amino)-2- ethylbenzoyl)piperazin-1- yl)propan-1-one | 601.6 | 602.4 |
| 776 | 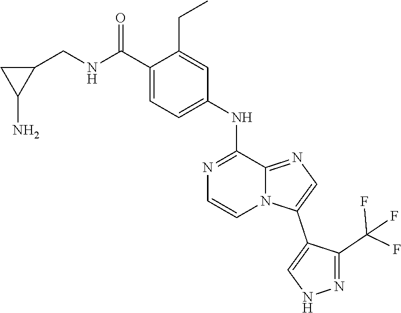 [SMILES: CCc1cc(Nc2nccn3c(-c4c[nH]nc4C(F)(F)F)cnc23)ccc1C(=O)NCC1CC1N] | N-((2-aminocyclopropyl) methyl)-2- ethyl-4-((3-(3- (trifluoromethyl)-1H- pyrazol-4-yl)imidazo [1,2-a]pyrazin- 8-yl)amino) benzamide formate | 484.5 | 485.3 |
| 777 | 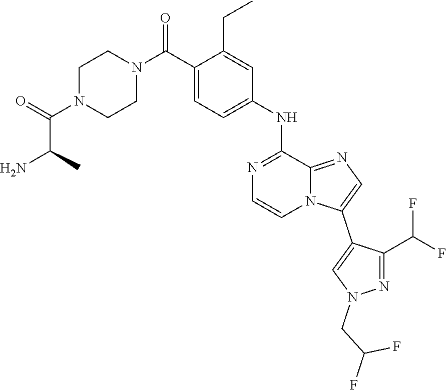 [SMILES: CCc1cc(Nc2nccn3c(-c4cn(CC(F)F)nc4C(F)F)cnc23)ccc1C(=O)N1CCN(C(=O)[C@@H](C)N)CC1] | (R)-2-amino-1- (4-(4-((3-(1-(2,2- difluoroethyl)-3- (difluoromethyl)- 1H-pyrazol-4-yl) imidazo[1,2- a]pyrazin-8-yl)amino)-2- ethylbenzoyl)piperazin-1- yl)propan-1-one | 601.6 | 602.3 |
| 778 | 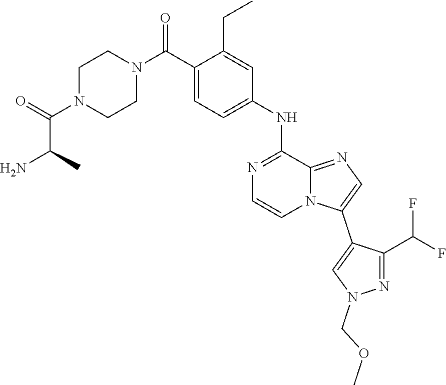 [SMILES: CCc1cc(Nc2nccn3c(-c4cn(COC)nc4C(F)F)cnc23)ccc1C(=O)N1CCN(C(=O)[C@@H](C)N)CC1] | (R)-2-amino-1-(4-(4-((3-(3- (difluoromethyl)-1- (methoxymethyl)- 1H-pyrazol-4- yl)imidazo[1,2-a]pyrazin-8- yl)amino)-2- ethylbenzoyl)piperazin-1- yl)propan-1-one | 581.6 | 582.4 |
| 779 | 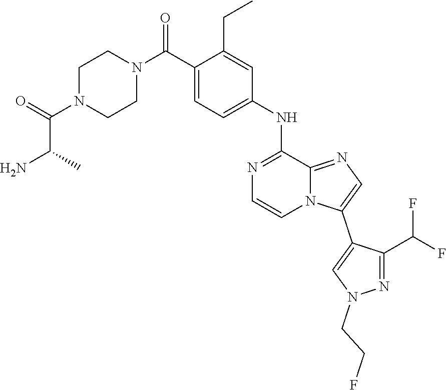 [SMILES: CCc1cc(Nc2nccn3c(-c4cn(CCF)nc4C(F)F)cnc23)ccc1C(=O)N1CCN(C(=O)[C@H](C)N)CC1] | (S)-2-amino-1-(4-(4-((3-(3- (difluoromethyl)- 1-(2-fluoroethyl)- 1H-pyrazol-4-yl)imidazo[1,2- a]pyrazin-8-yl)amino)-2- ethylbenzoyl)piperazin-1- yl)propan-1-one | 583.6 | 584.3 |
| 780 | 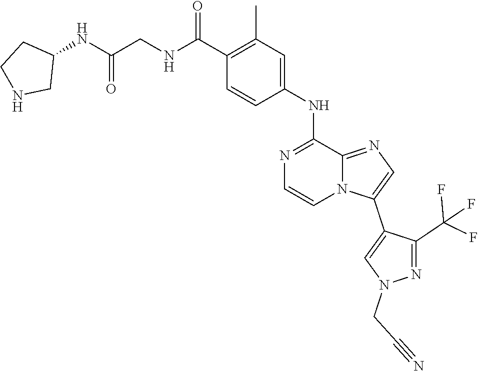 [SMILES: Cc1cc(Nc2nccn3c(-c4cn(CC#N)nc4C(F)(F)F)cnc23)ccc1C(=O)NCC(=O)N[C@H]1CCNC1] | 4-[[3-[1-(cyanomethyl)-3- (trifluoromethyl)pyrazol-4- yl]imidazo[1,2-a]pyrazin-8- yl]amino]-2-methyl-N-[2-oxo-2- [[(3S)-pyrrolidin-3- yl]amino]ethyl] benzamide formate | 566.5 | 567.2 |
| 781 | 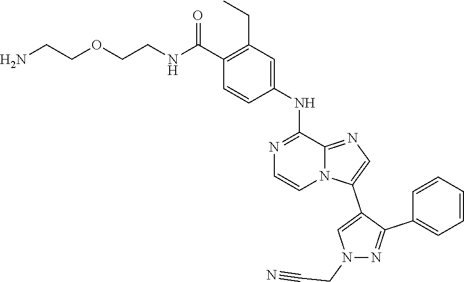 [SMILES: CCc1cc(Nc2nccn3c(-c4cn(CC#N)nc4-c4ccccc4)cnc23)ccc1C(=O)NCCOCCN] | N-[2-(2-aminoethoxy) ethyl]-4-[[3- [1-(cyanomethyl)-3- phenyl-pyrazol- 4-yl]imidazo[1,2-a]pyrazin-8- yl]amino]-2-ethyl-benzamide formate | 549.6 | 550.1 |
| 782 | 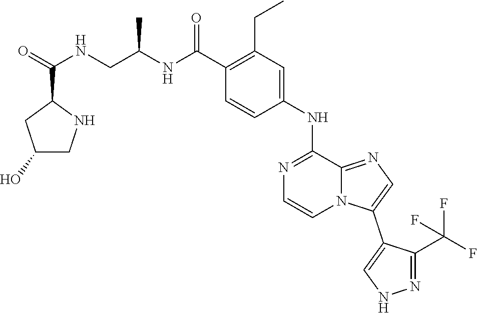 [SMILES: CCc1cc(Nc2nccn3c(-c4c[nH]nc4C(F)(F)F)cnc23)ccc1C(=O)N[C@H](C)CNC(=O)[C@@H]1C[C@@H](O)CN1] | (2S,4R)-N-((R)-2- (2-ethyl-4-((3-(3- (trifluoromethyl)- 1H-pyrazol-4- yl)imidazo[1,2-a]pyrazin-8- yl)amino)benzamido) propyl)-4- hydroxypyrrolidine-2- carboxamide | 585.6 | 586.4 |
| 783 | 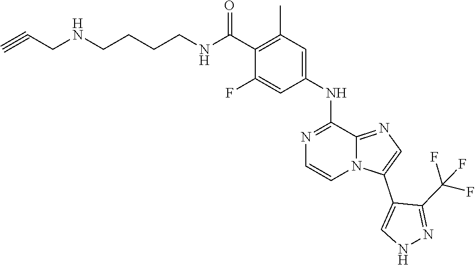 [SMILES: C#CCNCCCCNC(=O)c1c(C)cc(Nc2nccn3c(-c4c[nH]nc4C(F)(F)F)cnc23)cc1F] | 2-fluoro-6-methyl- N-(4-(prop-2-yn- 1-ylamino)butyl)-4-((3-(3- (trifluoromethyl)- 1H-pyrazol-4- yl)imidazo[1,2-a]pyrazin-8- yl)amino)benzamide | 528.5 | 529.3 |
| 784 | 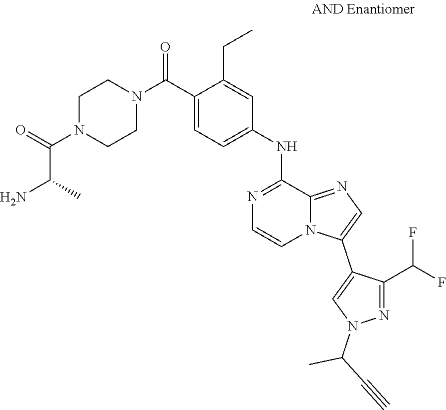 [SMILES: C#CC(C)n1cc(-c2cnc3c(Nc4ccc(C(=O)N5CCN(C(=O)[C@H](C)N)CC5)c(CC)c4)nccn23)c(C(F)F)n1] | (2R)-2-amino-1-(4- (4-((3-(1-(but-3- yn-2-yl)-3- (difluoromethyl)-1H- pyrazol-4-yl)imidazo [1,2-a]pyrazin- 8-yl)amino)-2- ethylbenzoyl)piperazin-1- yl)propan-1-one (mixture of epimers) | 589.6 | 590.4 |
| 785 | 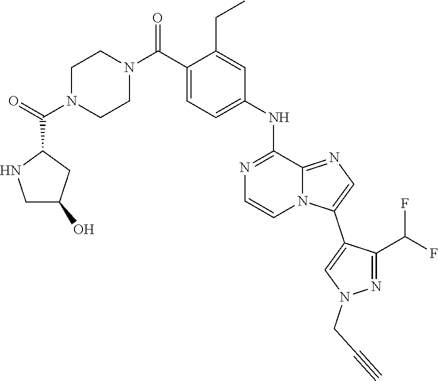 [SMILES: C#CCn1cc(-c2cnc3c(Nc4ccc(C(=O)N5CCN(C(=O)[C@@H]6C[C@@H](O)CN6)CC5)c(CC)c4)nccn23)c(C(F)F)n1] | (4-(4-((3-(3- (difluoromethyl)-1- (prop-2-yn-1-yl)- 1H-pyrazol-4- yl)imidazo[1,2-a]pyrazin-8- yl)amino)-2- ethylbenzoyl)piperazin-1- yl)((2S,4R)-4- hydroxypyrrolidin-2- yl)methanone | 617.7 | 618.3 |
| 786 | 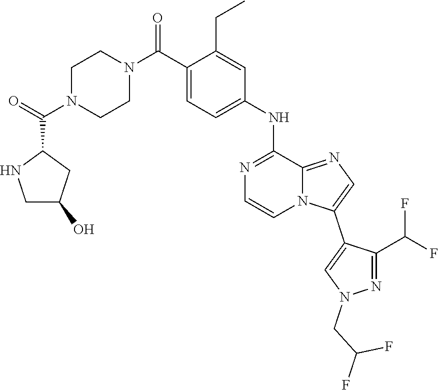 [SMILES: CCc1cc(Nc2nccn3c(-c4cn(CC(F)F)nc4C(F)F)cnc23)ccc1C(=O)N1CCN(C(=O)[C@@H]2C[C@@H](O)CN2)CC1] | (4-(4-((3-(1-(2,2- difluoroethyl)-3- (difluoromethyl)-1H- pyrazol-4- yl)imidazo[1,2-a]pyrazin-8- yl)amino)-2- ethylbenzoyl)piperazin-1- yl)((2S,4R)-4- hydroxypyrrolidin-2- yl)methanone | 643.6 | 644.4 |
| 787 | 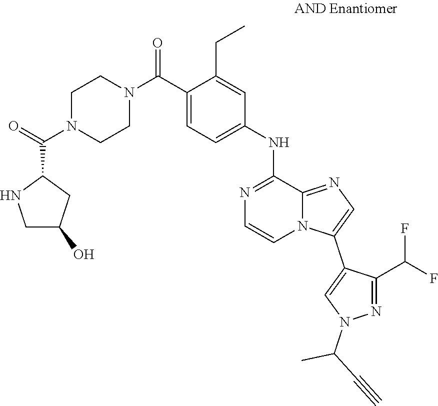 [SMILES: C#CC(C)n1cc(-c2cnc3c(Nc4ccc(C(=O)N5CCN(C(=O)[C@@H]6C[C@@H](O)CN6)CC5)c(CC)c4)nccn23)c(C(F)F)n1] | (4-(4-((3-(1-(but-3- yn-2-yl)-3- (difluoromethyl)- 1H-pyrazol-4- yl)imidazo[1,2-a] pyrazin-8- yl)amino)-2- ethylbenzoyl)piperazin-1- yl)((2R,4S)-4- hydroxypyrrolidin-2- yl)methanone (mixture of epimers) | 631.7 | 632.4 |
| 788 | 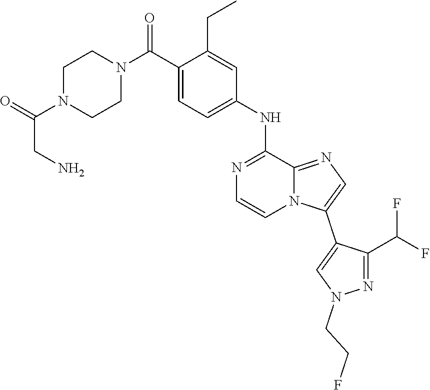 [SMILES: CCc1cc(Nc2nccn3c(-c4cn(CCF)nc4C(F)F)cnc23)ccc1C(=O)N1CCN(C(=O)CN)CC1] | 2-amino-1-(4-(4-((3-(3- (difluoromethyl)-1- (2-fluoroethyl)- 1H-pyrazol-4-yl)imidazo[1,2- a]pyrazin-8-yl)amino)-2- ethylbenzoyl)piperazin- 1-yl)ethan- 1-one | 569.6 | 570.4 |
| 789 | 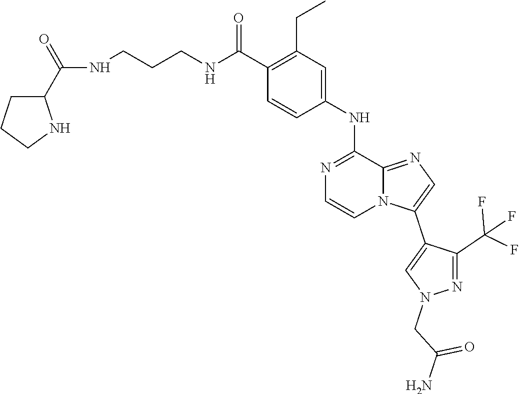 [SMILES: CCc1cc(Nc2nccn3c(-c4cn(CC(N)=O)nc4C(F)(F)F)cnc23)ccc1C(=O)NCCCNC(=O)C1CCCN1] | (S)-N-(3-(4-((3- (1-(2-amino-2- oxoethyl)-3- (trifluoromethyl)-1H- pyrazol-4-yl)imidazo [1,2-a]pyrazin- 8-yl)amino)-2- ethylbenzamido) propyl)pyrrolidine- 2-carboxamide | 626.6 | 627.3 |
| 790 | 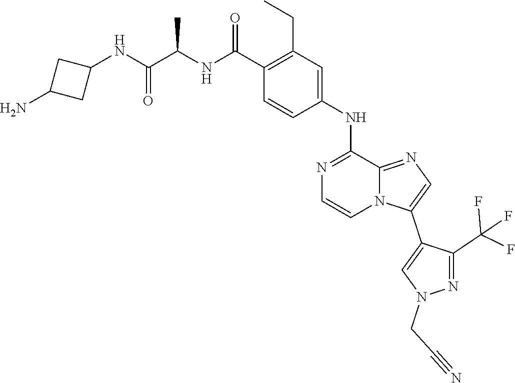 [SMILES: CCc1cc(Nc2nccn3c(-c4cn(CC#N)nc4C(F)(F)F)cnc23)ccc1C(=O)N[C@H](C)C(=O)NC1CC(N)C1] | N-[(1R)-2-[(3- aminocyclobutyl) amino]-1-methyl- 2-oxo-ethyl]-4-[[3-[1- (cyanomethyl)-3- (trifluoromethyl)pyrazol-4- yl]imidazo[1,2-a]pyrazin-8- yl]amino]-2-ethyl-benzamide formate | 594.6 | 595.4 |
| 791 | 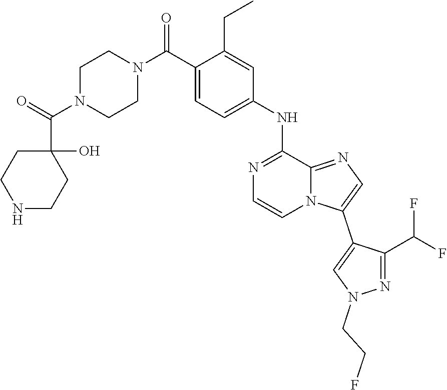 [SMILES: CCc1cc(Nc2nccn3c(-c4cn(CCF)nc4C(F)F)cnc23)ccc1C(=O)N1CCN(C(=O)C2(O)CCNCC2)CC1] | (4-(4-((3-(3- (difluoromethyl)-1-(2- fluoroethyl)-1H-pyrazol-4- yl)imidazo[1,2-a]pyrazin-8- yl)amino)-2- ethylbenzoyl) piperazin-1-yl)(4- hydroxypiperidin-4- yl)methanone | 639.7 | 640.4 |
| 792 | 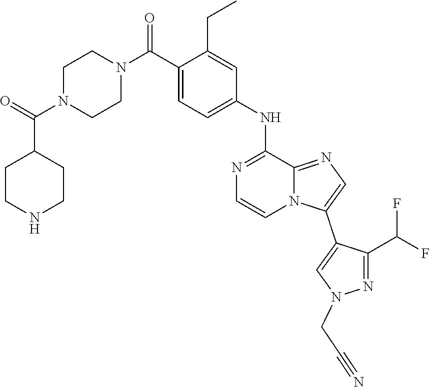 [SMILES: CCc1cc(Nc2nccn3c(-c4cn(CC#N)nc4C(F)F)cnc23)ccc1C(=O)N1CCN(C(=O)C2CCNCC2)CC1] | 2-(3-(difluoromethyl)- 4-(8-((3- ethyl-4-(4-(piperidine-4- carbonyl)piperazine-1- carbonyl)phenyl)amino) imidazo[1,2- a]pyrazin-3-yl)-1H-pyrazol-1- yl)acetonitrile | 616.7 | 617.4 |
| 793 | 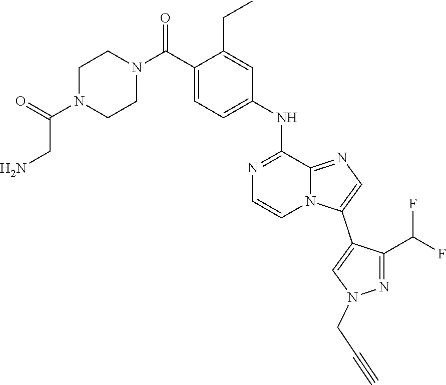 [SMILES: C#CCn1cc(-c2cnc3c(Nc4ccc(C(=O)N5CCN(C(=O)CN)CC5)c(CC)c4)nccn23)c(C(F)F)n1] | 2-amino-1-(4-(4-((3-(3- (difluoromethyl)- 1-(prop-2-yn-1- yl)-1H-pyrazol-4-yl) imidazo[1,2- a]pyrazin-8-yl)amino)-2- ethylbenzoyl)piperazin- 1-yl)ethan- 1-one | 561.6 | 562.3 |
| 794 | 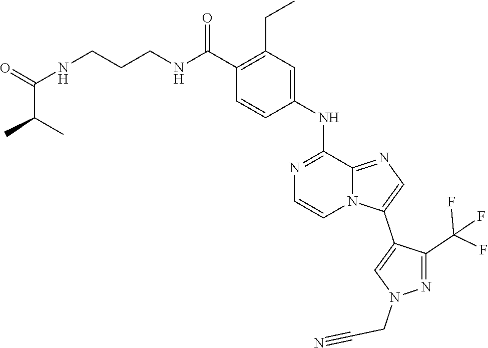 | (S)-N-(3-(2- aminopropanamido) propyl)-4-((3- (1-(cyanomethyl)-3- (trifluoromethyl)- 1H-pyrazol-4- yl)imidazo[1,2-a]pyrazin-8- yl)amino)-2-ethylbenzamide formate | 582.6 | 627.5 [M + HCOO]−, ESI neg |
| 795 | 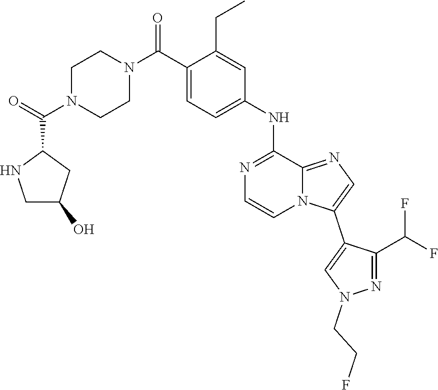 [SMILES: CCc1cc(Nc2nccn3c(-c4cn(CCF)nc4C(F)F)cnc23)ccc1C(=O)N1CCN(C(=O)[C@@H]2C[C@@H](O)CN2)CC1] | (4-(4-((3-(3- (difluoromethyl)-1-(2- fluoroethyl)-1H-pyrazol-4- yl)imidazo[1,2-a]pyrazin-8- yl)amino)-2- ethylbenzoyl)piperazin-1- yl)((2S,4R)-4- hydroxypyrrolidin-2- yl)methanone | 625.7 | 626.3 |
| 796 | 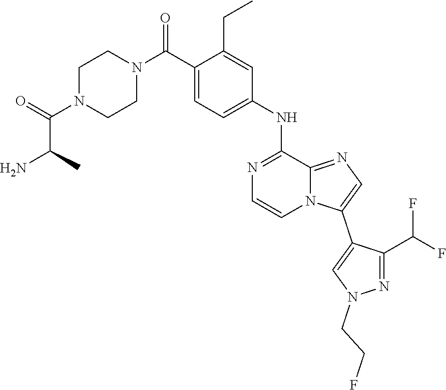 [SMILES: CCc1cc(Nc2nccn3c(-c4cn(CCF)nc4C(F)F)cnc23)ccc1C(=O)N1CCN(C(=O)[C@@H](C)N)CC1] | (R)-2-amino-1-(4-(4-((3-(3- (difluoromethyl)-1- (2-fluoroethyl)- 1H-pyrazol-4-yl)imidazo[1,2- a]pyrazin-8-yl)amino)-2- ethylbenzoyl)piperazin-1- yl)propan-1-one | 583.6 | 584.3 |
| 797 | 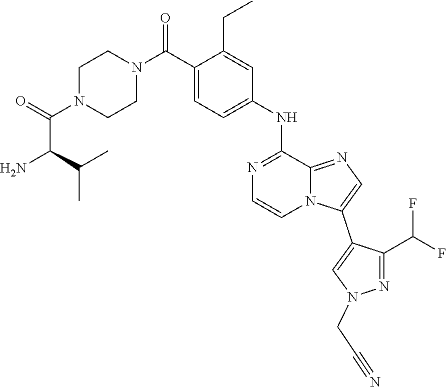 [SMILES: CCc1cc(Nc2nccn3c(-c4cn(CC#N)nc4C(F)F)cnc23)ccc1C(=O)N1CCN(C(=O)[C@H](N)C(C)C)CC1] | 2-(4-(8-((4-(4-(D-valyl) piperazine- 1-carbonyl)-3- ethylphenyl)amino) imidazo[1,2- a]pyrazin-3-yl)-3- (difluoromethyl)- 1H-pyrazol-1-yl)acetonitrile | 604.7 | 605.4 |
| 798 | 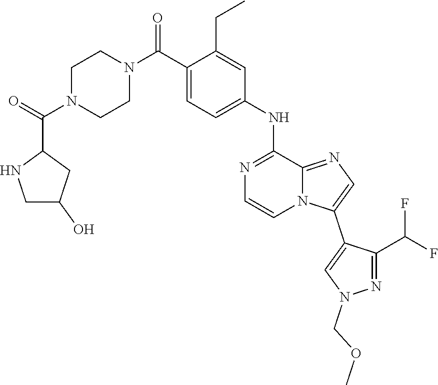 [SMILES: CCc1cc(Nc2nccn3c(-c4cn(COC)nc4C(F)F)cnc23)ccc1C(=O)N1CCN(C(=O)C2CC(O)CN2)CC1] | (4-(4-((3-(3- (difluoromethyl)-1- (methoxymethyl)- 1H-pyrazol-4- yl)imidazo[1,2- a]pyrazin-8- yl)amino)-2- ethylbenzoyl) piperazin-1-yl)(4- hydroxypyrrolidin-2- yl)methanone | 623.7 | 624.5 |
| 799 | 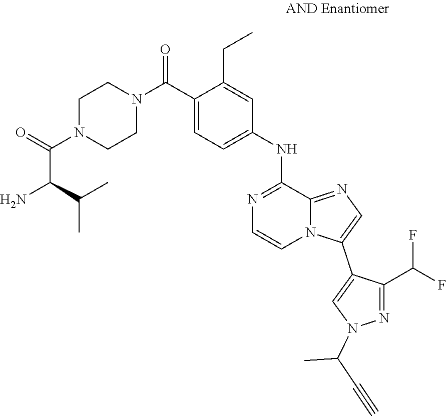 [SMILES: C#CC(C)n1cc(-c2cnc3c(Nc4ccc(C(=O)N5CCN(C(=O)[C@H](N)C(C)C)CC5)c(CC)c4)nccn23)c(C(F)F)n1] | (2R)-2-amino-1-(4- (4-((3-(1-(but-3- yn-2-yl)-3- (difluoromethyl)-1H- pyrazol-4-yl)imidazo [1,2-a]pyrazin- 8-yl)amino)-2- ethylbenzoyl) piperazin-1-yl)-3- methylbutan-1-one (mixture of epimers) | 617.7 | 618.4 |
| 800 | 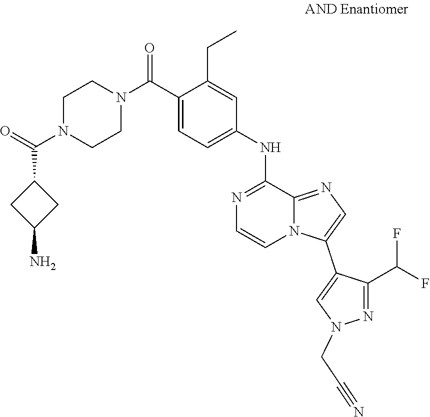 [SMILES: CCc1cc(Nc2nccn3c(-c4cn(CC#N)nc4C(F)F)cnc23)ccc1C(=O)N1CCN(C(=O)[C@H]2C[C@H](N)C2)CC1] | 2-(4-(8-((4-(4-((1r,3r)-3- aminocyclobutane-1- carbonyl)piperazine- 1-carbonyl)-3- ethylphenyl)amino) imidazo[1,2- a]pyrazin-3-yl)-3- (difluoromethyl)- 1H-pyrazol-1-yl)acetonitrile | 602.6 | 603.4 |
| 801 | 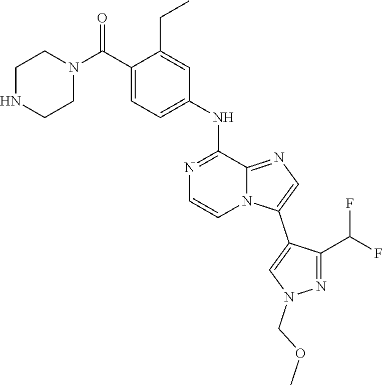 [SMILES: CCc1cc(Nc2nccn3c(-c4cn(COC)nc4C(F)F)cnc23)ccc1C(=O)N1CCNCC1] | (4-((3-(3- (difluoromethyl)-1- (methoxymethyl)- 1H-pyrazol-4- yl)imidazo[1,2-a] pyrazin-8- yl)amino)-2-ethylphenyl) (piperazin- 1-yl)methanone | 510.5 | 511.3 |
| 802 | 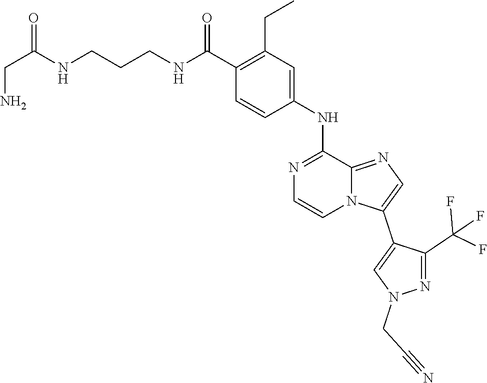 [SMILES: CCc1cc(Nc2nccn3c(-c4cn(CC#N)nc4C(F)(F)F)cnc23)ccc1C(=O)NCCCNC(=O)CN] | N-(3-(2-aminoacetamido) propyl)-4- ((3-(1-(cyanomethyl)-3- (trifluoromethyl)- 1H-pyrazol-4- yl)imidazo[1,2-a]pyrazin-8- yl)amino)-2-ethylbenzamide formate | 568.6 | 613.5 [M + HCOO]−, ESI neg |
| 803 | 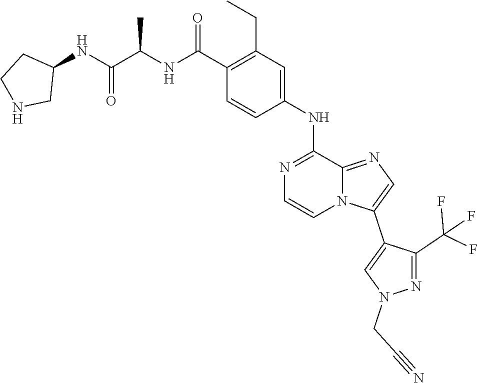 [SMILES: CCc1cc(Nc2nccn3c(-c4cn(CC#N)nc4C(F)(F)F)cnc23)ccc1C(=O)N[C@H](C)C(=O)N[C@@H]1CCNC1] | 4-[[3-[1-(cyanomethyl)-3- (trifluoromethyl)pyrazol-4- yl]imidazo[1,2-a]pyrazin-8- yl]amino]-2-ethyl-N-[(1R)-1- methyl-2-oxo-2- [[(3R)-pyrrolidin- 3-yl]amino]ethyl]benzamide formate | 594.6 | 595.4 |
| 804 | 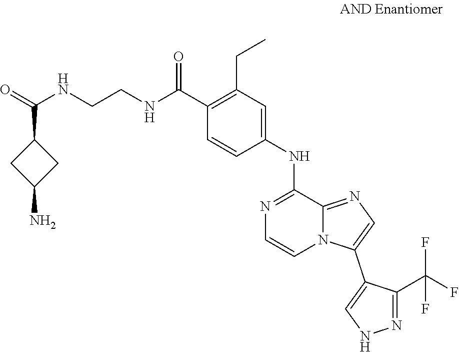 [SMILES: CCc1cc(Nc2nccn3c(-c4c[nH]nc4C(F)(F)F)cnc23)ccc1C(=O)NCCNC(=O)[C@H]1C[C@@H](N)C1] | N-(2-((1s,3s)-3- aminocyclobutane- 1-carboxamido) ethyl)-2-ethyl-4-((3- (3-(trifluoromethyl)- 1H-pyrazol-4- yl)imidazo[1,2-a]pyrazin-8- yl)amino)benzamide formate | 555.6 | 556.3 |

### Example 518

### ((3-Aminopropyl)imino)(methyl)(2-methyl-4-((3-(3-(trifluoromethyl)-1H-pyrazol-4-yl)imidazo[1,2-α]pyrazin-8-yl)amino)phenyl)-λ⁶-sulfanone

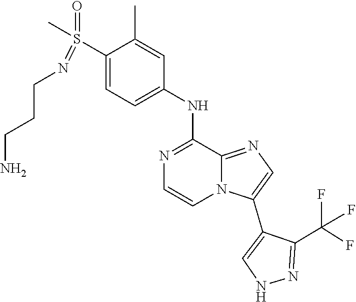 [SMILES: Cc1cc(Nc2nccn3c(-c4c[nH]nc4C(F)(F)F)cnc23)ccc1S(C)(=O)=NCCCN]

Step 1:

### (4-Bromo-2-methylphenyl)(imino)(methyl)-λ⁶-sulfanone

To a solution of (4-bromo-2-methylphenyl)(methyl)sulfane (4.4 g, 20.3 mmol, Eq: 1) in methanol (40.5 ml) was added (diacetoxyiodo)benzene (16.3 g, 50.7 mmol, Eq: 2.5) in portions and ammonium carbamate (3.16 g, 40.5 mmol, Eq: 2). The reaction mixture was stirred at room temperature for 4 hours (careful, strong exotherima after about 5 minutes!) Then the reaction mixture was concentrated in vacuo. The residue was treated with heptane and dichloromethane, the suspension filtered and washed with dichloromethane. The resulting solution was purified by silica gel chromatography using heptane/(EtOAc/EtOH/NH₄OH 75:25:2) as eluent. The title compound (3.695 g, 12.7 mmol, 62.5% yield) was obtained as yellow viscous oil with an assumed purity of 85% and was used without further purification. MS: 248.0, 250.0 [M+H]+, ESI pos, 1H NMR (300 MHz, CHLOROFORM-d) δ=8.02-7.93 (m, 1H), 7.57-7.45 (m, 2H), 3.13 (s, 3H), 2.74 (s, 3H)

Step 2:

### tert-Butyl (3-(((4-bromo-2-methylphenyl)(methyl)(oxo)-λ⁶-sulfaneylidene)amino)propyl)-carbamate

In a sealed tube as mixture of (4-bromo-2-methylphenyl)(imino)(methyl)-16-sulfanone (500 mg, 2.02 mmol, Eq: 1) and cesium carbonate (1.31 g, 4.03 mmol, Eq: 2) were combined with DMF (10 ml) was treated with tert-butyl (3-bromopropyl)carbamate (960 mg, 4.03 mmol, Eq: 2) and heated to 70° C. and stirred for 2 h. Then, again tert-butyl (3-bromopropyl)carbamate (480 mg, 2.02 mmol, Eq: 1) and cesium carbonate (657 mg, 2.02 mmol, Eq: 1) were added stirring continued for 20 h at 70° C. Then, again tert-butyl (3-bromopropyl)carbamate (480 mg, 2.02 mmol, Eq: 1) and cesium carbonate (657 mg, 2.02 mmol, Eq: 1) were added and the mixture stirred for 20 h at 70° C. Then again tert-butyl (3-bromopropyl)carbamate (480 mg, 2.02 mmol, Eq: 1) and cesium carbonate (657 mg, 2.02 mmol, Eq: 1) were added and the reaction mixture was stirred for 25 h at 70° C. The reaction mixture was poured into water and was extracted with EtOAc (2×). The organic layers were washed with brine, dried over sodium sulphate and concentrated in vacuo. The crude material was purified by silica gel chromatography using heptane/(EtOAc/EtOH/NH₄OH 75:25:2) as eluent. The obtained material was purified by preparative reversed phase HPLC (Column: YMC-Triart C18, 12 nm, 5 μm, 100×30 mm) using acetonitrile/water containing 0.1% triethylamine as eluent to afford the title compound (441 mg, 1.07 mmol, 52.9% yield) as light brown oil with a purity of 97.9% (total UV). MS: 407.1 [M+H]+, ESI pos

Step 3:

### 8-Chloro-3-(1-(methoxymethyl)-3-(trifluoromethyl)-1H-pyrazol-4-yl)imidazo[1,2-α]pyrazine

To a solution of 8-chloro-3-(3-(trifluoromethyl)-1H-pyrazol-4-yl)imidazo[1,2-α]pyrazine (example 4, step 1, 1.165 g, 4.05 mmol, Eq: 1) in DMF (20 ml) was added potassium carbonate (1.68 g, 12.2 mmol, Eq: 3) and chloro(methoxy)methane (652 mg, 615 μl, 8.1 mmol, Eq: 2) at 0° C. The reaction mixture was stirred for 20 h at RT. The reaction mixture was quenched with cold water and partitioned between ethyl acetate and water. The aqueous layer was extracted once more with ethyl acetate and the combined organic layers were washed with brine, dried over anhydrous sodium sulfate and concentrated in vacuo. The crude material was purified by silica gel chromatography using heptane/(EtOAc/EtOH/NH4OH 75:25:2) as eluent to afford the title compound (460 mg, 1.18 mmol, 29.1% yield) as light brown oil with a purity of 85% (total UV). MS: 332.1 [M+H]+, ESI pos.

Step 4:

### 3-(1-(Methoxymethyl)-3-(trifluoromethyl)-1H-pyrazol-4-yl)imidazo[1,2-α]pyrazin-8-amine

To a solution of 8-chloro-3-(1-(methoxymethyl)-3-(trifluoromethyl)-1H-pyrazol-4-yl)imidazo[1,2-α]pyrazine (830 mg, 2.5 mmol, Eq: 1) in isopropanol (10 ml) in a pressure tube was added 25% aq. ammonia (15.8 g, 17.5 ml, 231 mmol, Eq: 92.4), the tubes sealed and the reaction heated to 115° C. (high pressure!) over night. The reaction mixture was diluted with water, filtered and washed with water and heptane. The solid was collected and dried in vacuo. The crude material was purified by silica gel chromatography using heptane/(EtOAc/EtOH/NH₄OH 75:25:2) as eluent to afford the title compound (433.7 mg, 1.33 mmol, 53.3% yield) as off-white solid with a purity of 96%. MS: 313.1 [M+H]+, ESI pos.

Step 5:

### tert-Butyl (3-(((4-((3-(1-(methoxymethyl)-3-(trifluoromethyl)-1H-pyrazol-4-yl)imidazo[1,2-α]pyrazin-8-yl)amino)-2-methylphenyl)(methyl)(oxo)-λ⁶-sulfaneylidene)amino)propyl)carbamate

A mixture of 3-(1-(methoxymethyl)-3-(trifluoromethyl)-1H-pyrazol-4-yl)imidazo[1,2-α]pyrazin-8-amine (57.8 mg, 185 μmol, Eq: 1.5), tert-butyl (3-(((4-bromo-2-methylphenyl)(methyl)(oxo)-λ⁶-sulfaneylidene)amino)propyl)carbamate (50 mg, 123 μmol, Eq: 1) and potassium phosphate (78.5 mg, 370 μmol, Eq: 3) in dry 1,4-dioxane (3.56 ml) in a pressure tube was sparged with argon for 10 min while sonicating in the ultrasonic bath. Then Josiphos SL-J009-1 Pd G3 [1702311-34-9] (47.9 mg, 86.3 μmol, Eq: 0.7) was added and the tube was sparged again for 2 min. The reaction mixture was heated to 110° C. and stirred for 30 h. The reaction mixture was partitioned between ethyl acetate and water/brine (1:1). The aqueous layer was extracted two more times with ethyl acetate. The combined organic layers were dried over anhydrous sodium sulfate and concentrated in vacuo. The crude material was purified twice by silica gel chromatography using dichloromethane/methanol as eluent. The obtained material was purified by amine silica gel chromatography using heptane/(EtOAc/EtOH/NH₄OH 75:25:2) to afford the title compound (67.3 mg, 82.4 μmol, 66.8% yield) as orange oil with a purity of 78% (total UV). MS: 637.3 [M+H]+, ESI pos.

Step 6:

### ((3-Aminopropyl)imino)(methyl)(2-methyl-4-((3-(3-(trifluoromethyl)-1H-pyrazol-4-yl)imidazo[1,2-α]pyrazin-8-yl)amino)phenyl)-λ⁶-sulfanone

To a solution of tert-butyl (3-(((4-((3-(1-(methoxymethyl)-3-(trifluoromethyl)-1H-pyrazol-4-yl)imidazo[1,2-α]pyrazin-8-yl)amino)-2-methylphenyl)(methyl)(oxo)-λ⁶-sulfaneylidene)amino)propyl)carbamate (67.3 mg, 106 μmol, Eq: 1) in dioxane (530 μL) was added 4M HCl in dioxane (1.19 ml, 4.76 mmol, Eq: 45) and the reaction mixture stirred for 2 h at room temperature. Then water (10 μL) was added and the reaction mixture heated to 40° C. and stirred for 16 h. The reaction was heated to 60° C. and stirred for 2 h. Again, 4M HCl in dioxane (500 μL, 2 mmol, Eq: 18.9) was added and stirring continued for 16 h at 60° C. The reaction mixture was concentrated in vacuo. The residue was diluted with ethyl acetate, 1M aqueous sodium carbonate solution and brine (pH 9-10). The mixture was extracted 2× with ethyl acetate and the organic layers were washed 1× with brine. The combined organic layers were dried with sodium sulfate, filtered and concentrated in vacuo. The crude material was purified by silica gel chromatography using dichloromethane/(CH2Cl2/MeOH/NH4OH 90:10:1) as eluent and then (CH2Cl2/MeOH/NH4OH 80:20:5). The obtained material was resuspended in dichloromethane/methanol 90:10, filtered and concentrated in vacuo afford the title compound (24.5 mg, 48.4 μmol, 45.8% yield) as off-white solid with a purity of 97.3% (total UV). MS: 493.2 [M+H]+, ESI pos.

### Example 596

### N-[4-[N-(3-aminopropyl)-S-methyl-sulfonimidoyl]-3-methyl-phenyl]-3-[1-(cyclopropylmethyl)-3-(trifluoromethyl)pyrazol-4-yl]imidazo[1,2-α]pyrazin-8-amine formate

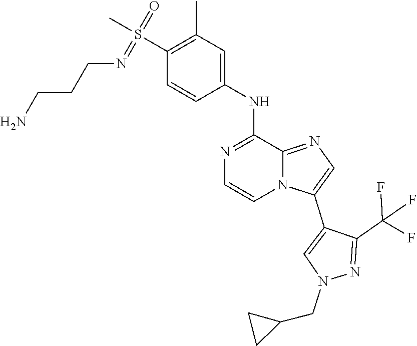 [SMILES: Cc1cc(Nc2nccn3c(-c4cn(CC5CC5)nc4C(F)(F)F)cnc23)ccc1S(C)(=O)=NCCCN]

Step 1:

### 2-Methyl-1-methylsulfanyl-4-nitro-benzene

A solution of 2-fluoro-5-nitrotoluene (20.0 g, 128.92 mmol, 1 eq) in DMF (80 mL) was added sodium thiomethoxide (8.13 g, 116.03 mmol, 0.900 eq) and stirred at 90° C. for 16 h. The mixture was poured into water and extracted 2× with EtOAc washed 3× with brine, concentrated and purified by silica column (PE/EA=20:1) to afford the title compound (16.58 g, 90.49 mmol, 70.19% yield) as yellow solid.

Step 2:

### Imino-methyl-(2-methyl-4-nitro-phenyl)-oxo-λ⁶-sulfane

A mixture of ammonium carbamate (4.05 g, 51.82 mmol, 1.5 eq), 2-methyl-1-methylsulfanyl-4-nitro-benzene (6.33 g, 34.55 mmol, 1 eq), iodobenzene diacetate (22.26 g, 69.09 mmol, 2 eq) in methanol (100 mL) was stirred at 10° C. for 16 h. The mixture was concentrated, the residue diluted with water, extracted 3× with EtOAc, washed with brine, dried over sodium sulfate and concentrated in vacuo. The crude material was purified by silica gel chromatography eluting with Petroleum ether/EtOAc to afford the title compound (5.25 g 24.51 mmol, 70.93% yield) as yellow oil. MS(m/e): 215.1 (M+H)+

Step 3:

### tert-Butyl N-[3-[[methyl-(2-methyl-4-nitro-phenyl)-oxo-λ⁶-sulfanylidene]amino]propyl]-carbamate

A mixture of imino-methyl-(2-methyl-4-nitro-phenyl)-oxo-λ⁶-sulfane (2.9 g, 13.54 mmol, 1 eq) and cesium carbonate (8.82 g, 27.07 mmol, 2 eq) in DMF (15 mL) was added 3-(BOC-amino)propyl bromide (2.62 mL, 20.3 mmol, 1.5 eq) and stirred at 50° C. for 16 h. The mixture was poured into water, extracted 3× with EtOAc, washed with brine, dried over sodium sulfate and concentrated in vacuo. The crude material was purified by silica gel chromatography eluting with Petroleum ether/EtOAc=20/1-1/1 to afford the title compound (3.6 g 9.69 mmol, 71.6% yield) as yellow solid. MS(m/e): 371.9 (M+H)+

Step 4:

### tert-Butyl N-[3-[[(4-amino-2-methyl-phenyl)-methyl-oxo-λ⁶-sulfanylidene]amino]propyl]carbamate

A mixture of nickel(II) chloride hexahydrate (1.15 g, 4.85 mmol, 0.500 eq) and sodium borohydride (183.33 mg. 4.85 mmol, 0.500 eq)in methanol (40 mL) was added tert-butyl N-[3-[[methyl-(2-methyl-4-nitro-phenyl)-oxo-λ⁶-sulfanylidene]amino]propyl]carbamate (3.6 g. 9.69 mmol, 1 eq) at 0° C., then sodium borohydride (1.1 g, 29.08 mmol, 3 eq) was added in position to the mixture and stirred for 1 h. The mixture was filtered and concentrated, purified by silica column (PE/EA=1:1) to afford the title compound (2.3 g, 6.74 mmol, 69.5% yield) as light yellow solid. MS(m/e): 342.2 (M+H)+

Step 5:

### 8-Chloro-3-[1-(cyclopropylmethyl)-3-(trifluoromethyl)pyrazol-4-yl]imidazo[1,2-α]pyrazine

To a solution of 8-chloro-3-[3-(trifluoromethyl)-1H-pyrazol-4-yl]imidazo[1,2-α]pyrazine (Example 4, Step 1, 300.0 mg, 1.04 mmol, 1 eq), potassium carbonate (288.3 mg, 2.09 mmol, 2 eq) in acetonitrile (15 mL) was added (bromomethyl)cyclopropane (0.2 mL, 2.09 mmol, 2 eq) at 10° C. and stirred for 16 h. The mixture was filtered and the obtained solution concentrated in vacuo. The residue was purified by prep-TLC (EtOAc/petroleum ether=1:1) to afford the the title compound (210 mg, 0.610 mmol, 58.92% yield) as white solid. MS(m/e): 342.5 (M+H)+

Step 6:

### tert-Butyl N-[3-[[[4-[[3-[1-(cyclopropylmethyl)-3-(trifluoromethyl)pyrazol-4-yl]imidazo[1,2-α]pyrazin-8-yl]amino]-2-methyl-phenyl]-methyl-oxo λ⁶-sulfanylidene]amino]propyl]carbamate

A stirred solution of tert-butyl N-[3-[[(4-amino-2-methyl-phenyl)-methyl-oxo-λ⁶-sulfanylidene]amino]propyl]carbamate (60 mg, 0.180 mmol, 1 eq), 8-chloro-3-[1-(cyclopropylmethyl)-3-(trifluoromethyl)pyrazol-4-yl]imidazo[1,2-α]pyrazine (60 mg, 0.180 mmol, 1 eq), tris(dibenzylideneacetone)dipalladium (0) (16.09 mg, 0.020 mmol, 0.100 eq), cesium carbonate (171.75 mg, 0.530 mmol, 3 eq) and 9,9-dimethyl-4,5-bis(diphenylphosphino)xanthene (10.17 mg, 0.020 mmol, 0.100 eq) in 1,4-dioxane (3 mL) was stirred at 115° C. under microwave irradiation for 2 h. The mixture was filtered and concentrated and the residue purified by prep-TLC (dichloromethane/methanol=10:1) to obtain the title compound (110 mg, 0.170 mmol, 96.8% yield) as yellow oil. 647.3 (M+H)+

Step 7:

### N-[4-[N-(3-Aminopropyl)-S-methyl-sulfonimidoyl]-3-methyl-phenyl]-3-[1-(cyclopropylmethyl)-3-(trifluoromethyl)pyrazol-4-yl]imidazo[1,2-α]pyrazin-8-amine formate

A solution of tert-butyl N-[3-[[[4-[[3-[1-(cyclopropylmethyl)-3-(trifluoromethyl)pyrazol-4-yl]imidazo[1,2-α]pyrazin-8-yl]amino]-2-methyl-phenyl]-methyl-oxo-λ⁶-sulfanylidene]amino]propyl]carbamate (110.0 mg, 0.170 mmol, 1 eq) in trifluoroacetic acid (2.0 mL, 25.96 mmol, 152.63 eq) and DCM (20 mL) was stirred at 10° C. for 16 h. The mixture was filtered and concentrated in vacuo. The residue was purified by prep-HPLC to afford the title compound (34.67 mg, 0.060 mmol, 37.29% yield) as yellow solid. MS(m/e): 547.2 (M+H)+

The following example was synthesized in analogy to procedures described before.

| | | | | MW |
|---|---|---|---|---|
| | | | Parent | (M + H) |
| No | Structure | Systematic Name | MW | found |
| 597 | 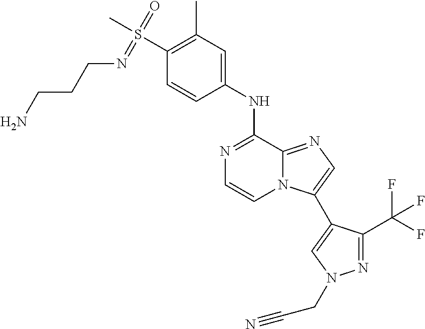 [SMILES: Cc1cc(Nc2nccn3c(-c4cn(CC#N)nc4C(F)(F)F)cnc23)ccc1S(C)(=O)=NCCCN] | 2-[4-[8-[4-[N-(3-aminopropyl)-S- methyl-sulfonimidoyl]-3-methyl- anilino]imidazo[1,2-a]pyrazin-3- yl]-3-(trifluoromethyl)pyrazol-1- yl]acetonitrile | 531.6 | 532.2 |

### Example 805

### (2-fluoro-4-((3-(3-(trifluoromethyl)-1H-pyrazol-4-yl)imidazo[1,2-α]pyrazin-8-yl)amino)phenyl)(piperazin-1-yl)methanone hydrochloride

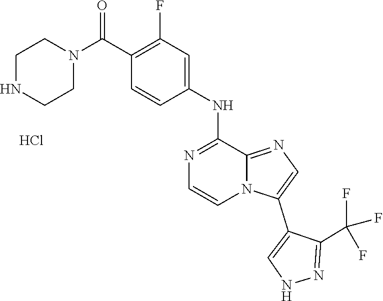 [SMILES: O=C(c1ccc(Nc2nccn3c(-c4c[nH]nc4C(F)(F)F)cnc23)cc1F)N1CCNCC1 ; Cl]

Step 1:

### methyl-2-fluoro-4-((3-(3-(trifluoromethyl)-1H-pyrazol-4-yl)imidazo[1,2-α]pyrazin-8-yl)amino)benzoate

A mixture of 8-chloro-3-(3-(trifluoromethyl)-1H-pyrazol-4-yl)imidazo[1,2-α]pyrazine (500 mg, 1.74 mmol, Eq: 1), methyl 4-amino-2-fluorobenzoate (368 mg, 2.17 mmol, Eq: 1.25) in Acetonitrile (12 ml) and Acetic Acid (1.2 ml) was stirred for 3 h at 120° C. After cooling to room temperature the precipitate was filtered off, washed with acetonitrile/methanol 1:1 and dried to yield the title compound as off-white crystals (353.7 mg, 799 μmol, 46% yield). MS(m/e): 421.2 (M+H).

Step 2:

### 2-fluoro-4-((3-(3-(trifluoromethyl)-1H-pyrazol-4-yl)imidazo[1,2-α]pyrazin-8-yl)amino)benzoic acid

A mixture of methyl 2-fluoro-4-((3-(3-(trifluoromethyl)-1H-pyrazol-4-yl)imidazo[1,2-α]pyrazin-8-yl)amino)benzoate (353.7 mg, 841 μmol, Eq: 1) and LiOH·H₂O (70.6 mg, 1.68 mmol, Eq: 2) in THF (5 mL), water (2.5 mL) and MeOH (0.4 mL) was stirred for 2 h at room temperature. Then 1M NaOH (841 μl, 841 μmol, Eq: 1) was added and the solution was stirred for another 3 h. The reaction mixture was concentrated in vacuo. Under ice colling, 2.25 ml of an aqueous solution of HCl 1.0 M was added dropwise. The precipitate was filtered through sintered glass, washed with water and dried under high vacuum to give the title compound as white solid (314 mg, 757 μmol, 90%). MS(m/e): 407.1 (M+H).

Step 3:

### tert-butyl 4-(2-fluoro-4-((3-(3-(trifluoromethyl)-1H-pyrazol-4-yl)imidazo[1,2-α]pyrazin-8-yl)amino)benzoyl)piperazine-1-carboxylate

A mixture of 2-fluoro-4-((3-(3-(trifluoromethyl)-1H-pyrazol-4-yl)imidazo[1,2-α]pyrazin-8-yl)amino)benzoic acid (134 mg, 330 μmol, Eq: 1), HATU (144 mg, 379 μmol, Eq: 1.15), tert-butyl piperazine-1-carboxylate (61.4 mg, 330 μmol, Eq: 1) and DIPEA (256 mg, 346 μl, 1.98 mmol, Eq: 6) in DMF (3 mL) was stirred at room temperature overnight. The residue was purified by preparative HPLC to yield after evaporation of the product containing fractions the title compound as off white solid (111.2 mg, 56.7%). MS(m/e): 575.2 (M+H).

Step 4:

### (2-fluoro-4-((3-(3-(trifluoromethyl)-1H-pyrazol-4-yl)imidazo[1,2-α]pyrazin-8-yl)amino)phenyl)(piperazin-1-yl)methanone hydrochloride

A solution of tert-butyl 4-(2-fluoro-4-((3-(3-(trifluoromethyl)-1H-pyrazol-4-yl)imidazo[1,2-α]pyrazin-8-yl)amino)benzoyl)piperazine-1-carboxylate (111.2 mg, 194 μmol, Eq: 1) in DCM (10 ml) and MeOH (2 ml), was treated with an excess of HCl 4 M in Dioxan (968 μl, 3.87 mmol, Eq: 20) and the mixture was stirred over night at room temperature. The mixture was concentrated in vacuo, triturated with diethylether (15 ml). The white solid was fitered off, triturated with diethylether (2 ml) and dried under reduced pressure to afford the title compound as off white solid (110.8 mg, 190 μmol, 98.1% yield). MS(m/e): 475.2 (M+H).

### Example 806

### (4-((1S,3R)-3-aminocyclopentane-1-carbonyl)piperazin-1-yl)(4-((3-(1-(2,2-difluoroethyl)-3-(trifluoromethyl)-1H-pyrazol-4-yl)imidazo[1,2-α]pyrazin-8-yl)amino)-2-fluorophenyl)methanone hydrochloride

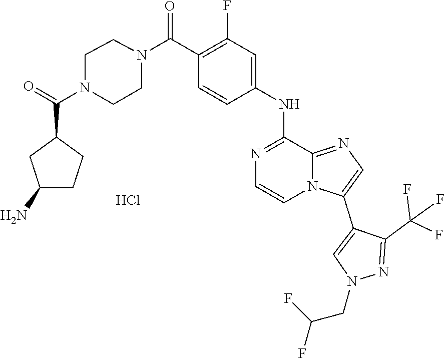 [SMILES: N[C@@H]1CC[C@H](C(=O)N2CCN(C(=O)c3ccc(Nc4nccn5c(-c6cn(CC(F)F)nc6C(F)(F)F)cnc45)cc3F)CC2)C1 ; Cl]

Step 1:

### tert-butyl ((1R,3S)-3-(4-(2-fluoro-4-((3-(3-(trifluoromethyl)-1H-pyrazol-4-yl)imidazo[1,2-α]pyrazin-8-yl)amino)benzoyl)piperazine-1-carbonyl)cyclopentyl)carbamate

A mixture of 2-fluoro-4-((3-(3-(trifluoromethyl)-1H-pyrazol-4-yl)imidazo[1,2-α]pyrazin-8-yl)amino)phenyl)(piperazin-1-yl)methanone hydrochloride (103.8 mg, 178 μmol, Eq: 1), HATU (84.5 mg, 222 μmol, Eq: 1.25), (1S,3R)-3-((tert-butoxycarbonyl)amino)cyclopentane-1-carboxylic acid (48.9 mg, 213 μmol, Eq: 1.2) and DIPEA (414 mg, 559 μl, 3.2 mmol, Eq: 18) in DMF (4 mL) was stirred at room temperature for 2 h. The residue was purified by preparative HPLC to yield after evaporation of the product containing fractions the title compound as off white solid (114.2 mg, 93.7%). MS(m/e): 686.5 (M+H).

Step 2:

### tert-butyl ((1R,3S)-3-(4-(4-((3-(1-(2,2-difluoroethyl)-3-(trifluoromethyl)-1H-pyrazol-4-yl)imidazo[1,2-α]pyrazin-8-yl)amino)-2-fluorobenzoyl)piperazine-1-carbonyl)cyclopentyl)carbamate

A mixture of tert-butyl ((1R,3S)-3-(4-(2-fluoro-4-((3-(3-(trifluoromethyl)-1H-pyrazol-4-yl)imidazo[1,2-α]pyrazin-8-yl)amino)benzoyl)piperazine-1-carbonyl)cyclopentyl)carbamate (28.55 mg, 41.6 μmol, Eq: 1), potassium carbonate (17.3 mg, 125 μmol, Eq: 3) and 1,1,1-trifluoro-2-iodoethane (26.2 mg, 125 μmol, Eq: 3) in DMF (1 mL) was stirred at room temperature overnight. The residue was purified by preparative HPLC to yield after evaporation of the product containing fractions the title compound as off white solid (17 mg, 54.5%). MS(m/e): 725.5 (M+H).

Step 3:

### (4-((1S,3R)-3-aminocyclopentane-1-carbonyl)piperazin-yl) (4-((3-(1-(2,2-difluoroethyl)-3-(trifluoromethyl)-1H-pyrazol-4-yl)imidazo[1,2-α]pyrazin-8-yl)amino)-2-fluorophenyl)methanone hydrochloride

A solution of tert-butyl ((1R,3S)-3-(4-(4-((3-(1-(2,2-difluoroethyl)-3-(trifluoromethyl)-1H-pyrazol-4-yl)imidazo[1,2-α]pyrazin-8-yl)amino)-2-fluorobenzoyl)piperazine-1-carbonyl)cyclopentyl)carbamate (29.9 mg, 40 μmol, Eq: 1) in DCM (2 ml) was treated with an excess of HCl 4 M in Dioxan (200 μl) and the mixture was stirred for 3 hr at room temperature.

The mixture was concentrated in vacuo, triturated with diethylether (5 ml). The white solid was fitered off, triturated with diethylether (1 ml) and dried under reduced pressure to afford the title compound as off white solid (19.3 mg, 66.7% yield). MS(m/e): 650.4 (M+H).

The following examples were synthesized in analogy to procedures described before. The reaction sequence and staring materials are outlined in the Table 5.

TABLE 5

| | | | Par- | MW | |
|---|---|---|---|---|---|
| | | Systematic | ent | (M + H) | Reaction Sequence and |
| No | Stucture | Name | MW | found | Starting Materials |
| 807 | 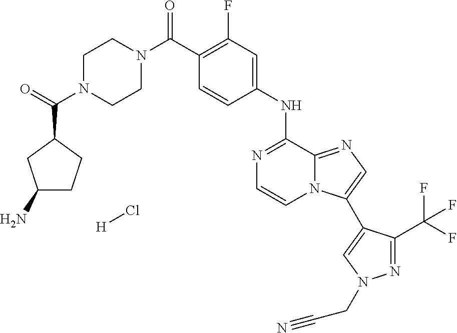 [SMILES: N#CCn1cc(-c2cnc3c(Nc4ccc(C(=O)N5CCN(C(=O)[C@H]6CC[C@@H](N)C6)CC5)c(F)c4)nccn23)c(C(F)(F)F)n1 ; Cl] | 2-(4-(8-((4-(4- ((1S,3R)-3- aminocyclo- pentane-1- carbonyl) piperazin- 1-carbonyl)-3- fluorophenyl) amino)imidazo [1,2-a] pyrazin-3-yl)-3- (trifluoromethyl)- 1H-pyrazol-1- yl)acetonitrile hydrochloride | 624.5 | 625.4 (M + H)+ | in analogy to the procedure for example 2: amide coupling of 2- fluoro-4-((3-(3- (trifluoromethyl)-1H- pyrazol-4-yl) imidazo[1,2- a]pyrazin-8- yl)amino)phenyl) (piperazin- 1-yl)methanone hydrochloride (example 1) with (1S,3R)- 3-((tert- butoxycarbonyl) amino)cyclopentane- 1-carboxylic acid, alkylation with 2- bromoacetonitrile and |
| | | | | | subsequent acidic Boc |
| | | | | | deprotection |
| 808 | 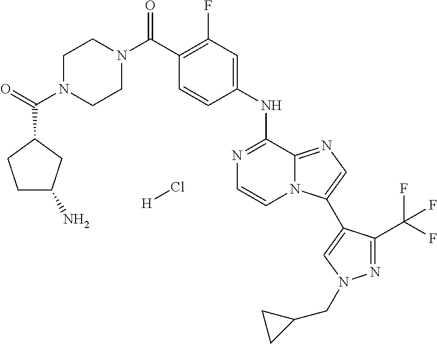 [SMILES: N[C@@H]1CC[C@H](C(=O)N2CCN(C(=O)c3ccc(Nc4nccn5c(-c6cn(CC7CC7)nc6C(F)(F)F)cnc45)cc3F)CC2)C1 ; Cl] | (4-((1S,3R)-3- aminocyclo- pentane-1- carbonyl) piperazin- 1-yl)(4-((3-(1- (cyclopropyl- methyl)-3- (trifluoromethyl)- 1H-pyrazol-4- yl)imidazo[1,2- a]pyrazin-8- yl)amino)-2- fluorophenyl) methanone hydrochloride | 639.5 | 640.4 (M + H)+ | in analogy to the procedure for example 2: amide coupling of 2- fluoro-4-((3-(3- (trifluoromethyl)-1H- pyrazol-4-yl) imidazo[1,2- a]pyrazin-8- yl)amino)phenyl) (piperazin- 1-yl)methanone hydrochloride (example 1) with (1S,3R)- 3-((tert- butoxycarbonyl)amino) cyclopentane-1- carboxylic acid, alkylation with (bromomethyl) cyclopropane and |
| | | | | | subsequent acidic |
| | | | | | Boc deprotection |
| 809 | 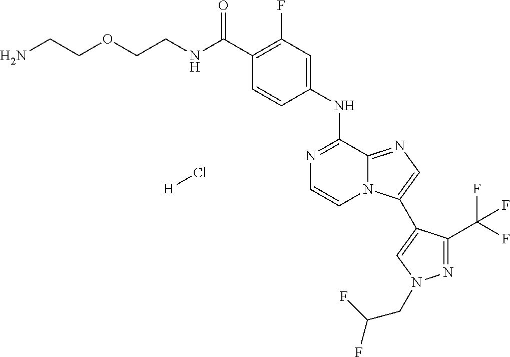 [SMILES: NCCOCCNC(=O)c1ccc(Nc2nccn3c(-c4cn(CC(F)F)nc4C(F)(F)F)cnc23)cc1F ; Cl] | N-(2-(2- aminoethoxy) ethyl)-4- ((3-(1-(2,2- difluoroethyl)-3- (trifluoromethyl)- 1H-pyrazol-4- yl)imidazo[1,2- a]pyrazin-8- yl)amino)-2- fluorobenzamidei hydrochloride | 556.3 | 657 3 (M + H)+ | in analogy to the procedure for example 1: amide coupling of 2- fluoro-4-((3-(3- (trifluoromethyl)-1H- pyrazol-4-yl) imidazo[1,2- a]pyrazin-8- yl)amino)benzoic acid (example 1) with tert- butyl (2-(2- aminoethoxy)ethyl) carbamate, alkylation with 1,1,1-trifluoro-2- iodoethane and subsequent acidic Boc deprotection |
| 810 | 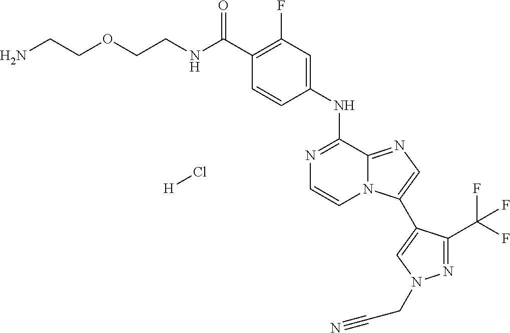 [SMILES: N#CCn1cc(-c2cnc3c(Nc4ccc(C(=O)NCCOCCN)c(F)c4)nccn23)c(C(F)(F)F)n1 ; Cl] | N-(2-(2- aminoethoxy) ethyl)-4-((3-(1- (cyanomethyl)-3- (trifluoromethyl)- 1H-pyrazol-4- yl)imrdazo[1,2- a]pyrazin-8- yl)amino)-2- fluorobenzamide hydrochloride | 531.3 | 532.3 (M + H)+ | in analogy to the procedure for example 1: amide coupling 2-fluoro- 4-((3-(3- (trifluoromethyl)- 1H-pyrazol-4- yl)imidazo [1,2-a]pyrazin- 8-yl)amino) benzoic acid (example 1) with tert- butyl (2-(2- aminoethoxy) ethyl)carbamate, alkylation with 2- bromoacetonitrile and subsequent acidic Boc deprotection |
| 811 | 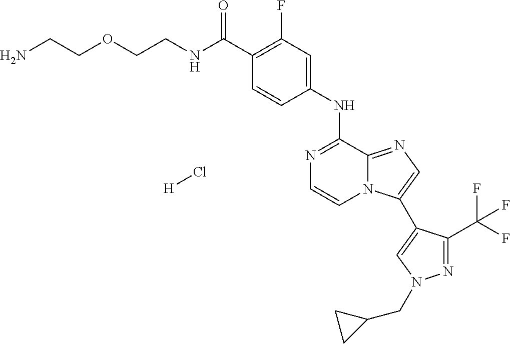 [SMILES: NCCOCCNC(=O)c1ccc(Nc2nccn3c(-c4cn(CC5CC5)nc4C(F)(F)F)cnc23)cc1F ; Cl] | N-(2-(2- aminoethoxy) ethyl)-4-((3-(1- (cyclopropyl- methyl)-3- (trifluoromethyl)- 1H-pyrazol-4- yl)imidazo[1,2- a]pyrazin-8- yl)amino)-2- fluorobenzamide hydrochloride | 546.4 | 547.3 (M + H)+ | in analogy to the procedure for example 1: amide coupling of coupling 2- fluoro-4-((3- (3-(trifluoromethyl)- 1H-pyrazol-4-yl) imidazo[1,2- a]pyrazin-8- yl)amino)benzoic acid (example 1) with tert- butyl (2-(2- aminoethoxy) ethyl)carba- mate, alkylation with (bromomethyl) cyclopropane and subsequent acidic Boc deprotection |
| 812 | 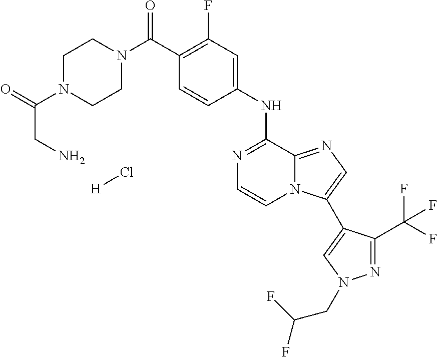 | 2-amino-1-(4-(4- (3-(1-2,2- difluoroethyl)-3- (trifluoromethyl)- 1H-pyrazol-4- yl)imidazo[1,2- a]pyrazin-8- yl)amino)-2- fluorobenzoyl) piperazin-1-yl) ethan- 1-one hydrochloride | 595.4 | 596.3 (M + H)+ | in analogy to the procedure for example 1: amide coupling coupling 2-fluoro-4-((3-(3- (trifluoromethyl)-1H- pyrazol-4-yl) imidazo[1,2- a]pyrazin-8- yl)amino)benzoic acid (example 1) with tert- butyl (2-oxo- 2-(piperazin- 1-yl)ethyl)carbamate, alkylation with 1,1,1- trifluoro-2- iodoethane and subsequent acidic Boc deprotection |
| 813 | 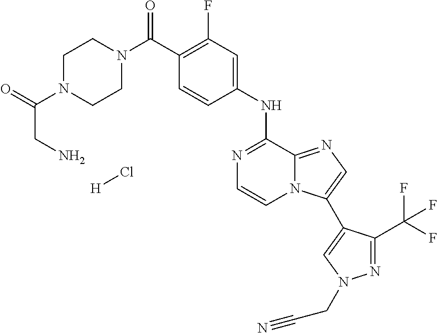 | 2-(4-(8-((3- fluoro-4-(4- glycylpiperazine- 1- carbonyl)phenyl) amino)imidazo [1,2-a] pyrazin-3-yl)- 3- (trifluoromethyl)- 1H-pyrazol-1- yl)acetonitrile hydrochloride | 570.4 | 571.3 (M + H)+ | in analogy to the procedure for example 1: amide coupling coupling 2-fluoro-4-((3-(3- (trifluoromethyl)-1H- pyrazol-4-yl) imidazo[1,2- a]pyrazin-8- yl)amino)benzoic acid (example 1), with tert- butyl (2-oxo- 2-(piperazin- 1-yl)ethyl)carbamate, alkylation with 2- bromoacetonitrile and subsequent acidic Boc deprotection |
| 814 | 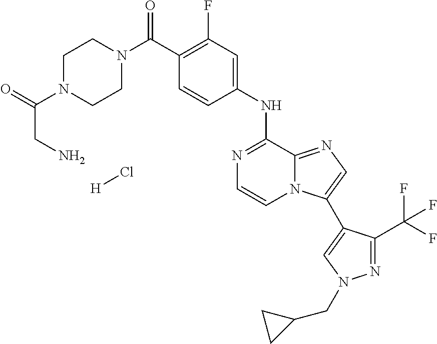 | 2-amino-1-(4-(4- ((3-(1- (cyclopropyl- methyl)-3- (trifluoromethyl)- 1H-pyrazol-4- yl)imidazo[1,2- a]pyrazin-8- yl)amino)-2- fluorobenzoyl) piperazin-1- yl)ethan-1-one hydrochloride | 585.4 | 586.3 (M + H)+ | in analogy to the procedure for example 1: amide coupling of coupling 2- fluoro-4-((3- (3-(trifluoromethyl)- 1H-pyrazol-4-yl) imidazo[1,2- a]pyrazin-8- yl)amino)benzoic acid (example 1),), with tert-butyl (2-oxo- 2-(piperazin- 1-yl)ethyl)carbamate, alkylation with (bromomethyl) cyclopropane and subsequent acidic Boc deprotection |

### Example 815

### N-(2-((2-((2-aminoethyl)amino)-2-oxoethyl)amino)ethyl)-2-chloro-4-((3-(1-(cyanomethyl)-3-(trifluoromethyl)-1H-pyrazol-4-yl)imidazo[1,2-α]pyrazin-8-yl)amino)benzamide formate

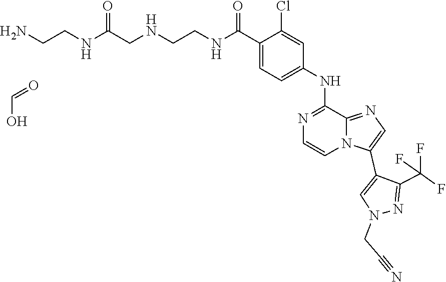 [SMILES: O=CO ; N#CCn1cc(-c2cnc3c(Nc4ccc(C(=O)NCCNCC(=O)NCCN)c(Cl)c4)nccn23)c(C(F)(F)F)n1]

Step 1:

### tert-butyl (2-(2-chloro-4-((3-(3-(fluoromethyl)-1H-pyrazol-4-yl)imidazo[1,2-α]pyrazin-8-yl)amino)benzamido)ethyl)carbamate formate

2-chloro-4-((3-(3-(trifluoromethyl)-1H-pyrazol-4-yl)imidazo[1,2-α]pyrazin-8-yl)amino)benzoic acid (2.5 g, 5.32 mmol, Eq: 1) was dissolved in DMF (25 ml). DIPEA (2.06 g, 2.79 ml, 16 mmol, Eq: 3), tert-butyl (2-aminoethyl)carbamate (1.02 g, 1.01 ml, 6.39 mmol, Eq: 1.2) and HATU (2.43 g, 6.39 mmol, Eq: 1.2) were added and the mixture was stirred at RT over 90 minutes. The crude material was purified by reverse phase chromatography to afford the title compound (2.62 g, 80.6%). MS(m/e): 563.3 (M−H)−.

Step 2:

### tert-butyl (2-(2-chloro-4-((3-(1-(cyanomethyl)-3-(trifluoromethyl)-1H-pyrazol-4-yl)imidazo[1,2-α]pyrazin-8-yl)amino)benzamido)ethyl)carbamate

tert-butyl (2-(2-chloro-4-((3-(3-(trifluoromethyl)-1H-pyrazol-4-yl)imidazo[1,2-α]pyrazin-8-yl)amino)benzamido)ethyl)carbamate formate (1.31 g, 2.14 mmol, Eq: 1) was dissolved in DMF (10 ml). 2-bromoacetonitrile (514 mg, 4.29 mmol, Eq: 2) and DIPEA (831 mg, 1.12 ml, 6.43 mmol, Eq: 3 were added and the mixture was stirred at room temperature for 64 hr.. The crude was purified by reverse phase chromatography to afford the title compound (1.15 g, 85.2%). MS(m/e): 648.3 (M+HCOO)—

Step 3:

### N-(2-aminoethyl)-2-chloro-4-((3-(1-(cyanomethyl)-3-(trifluoromethyl)-1H-pyrazol-4-yl)imidazo[1,2-α]pyrazin-8-yl)amino)benzamide formate

tert-butyl (2-(2-chloro-4-((3-(1-(cyanomethyl)-3-(trifluoromethyl)-1H-pyrazol-4-yl)imidazo[1,2-α]pyrazin-8-yl)amino)benzamido)ethyl)carbamate (1.15 g, 1.9 mmol, Eq: 1) was dissolved in DCM (15 ml) and treated with an excess of TFA (5.43 g, 3.67 ml, 47.6 mmol, Eq: 25) at room temperature over night. The reaction mixture was quenched by addition of NH₃ in MeOH. The volatils were evaporated and the crude product was purified by preparative HPLC to afford the title compound (776 mg, 51.9%). MS(m/e): 548.3 (M+HCOO)—.

Step 4:

### tert-butyl (2-(2-((2-(2-chloro-4-((3-(1-(cyanomethyl)-3-(trifluoromethyl)-1H-pyrazol-4-yl)imidazo[1,2-α]pyrazin-8-yl)amino)benzamido)ethyl)amino)acetamido)ethyl)carbamate

N-(2-aminoethyl)-2-chloro-4-((3-(1-(cyanomethyl)-3-(trifluoromethyl)-1H-pyrazol-4-yl)imidazo[1,2-α]pyrazin-8-yl)amino)benzamide formate (150 mg, 191 μmol, Eq: 1) was dissolved in DMF (2 ml). DIPEA (74 mg, 100 μl, 573 μmol, Eq: 3) and tert-butyl (2-(2-chloroacetamido)ethyl)carbamate (54.2 mg, 229 μmol, Eq: 1.2) were added and the reaction mixture was stirred at RT over 2 nights. The crude was purified by preparative HPLC to afford the title compound (50 mg, 27.9%). MS(m/e): 748.4 (M+HCOO)—.

Step 5:

### N-(2-((2-((2-aminoethyl)amino)-2-oxoethyl)amino)ethyl)-2-chloro-4-((3-(1-(cyanomethyl)-3-(trifluoromethyl)-1H-pyrazol-4-yl)imidazo[1,2-α]pyrazin-8-yl)amino)benzamide formate

tert-butyl (2-(2-((2-(2-chloro-4-(5-(1-(cyanomethyl)-3-(trifluoromethyl)-1H-pyrazol-4-yl)-1-methyl-1H-imidazole-2-carboxamido)benzamido)ethyl)amino)acetamido)ethyl)carbamate (50 mg, 0.066 mmol, Eq: 1)) was dissolved in DCM (1 ml) and treated with an excess of TFA (152 mg, 0.1 ml, 1.33 mmol, Eq: 20) at room temperature for 2 hr. The reaction mixture was neutralised with DCM/MeOH/NH₃. The volatils were evaporated and the crude product was purified by reverse phase chromatography to afford the title compound (5 mg, 12.5%). MS(m/e): 604.3 (M+H).

The following examples were synthesized in analogy to procedures described before. The reaction sequence and staring materials are outlined in Table 6.

TABLE 6

| | | | | MW | |
|---|---|---|---|---|---|
| | | | Parent | (M + H) | Reaction Sequence and |
| No | Stucture | Systematic Name | MW | found | Starting Materials |
| 816 | 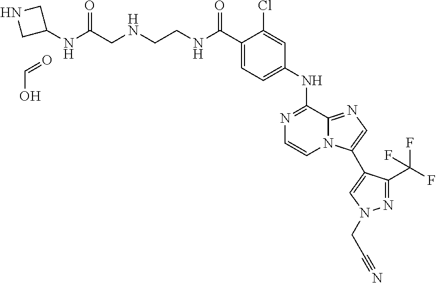 | N-(2-(2- (azetidin-3- ylamino)-2- oxoethyl)amino) ethyl)-2-chloro- 4-((3-(1- (cyanomethyl)-3- (trifluoromethyl)- 1H-pyrazol-4- yl)imidazo[1,2- a]pyrazin-8- yl)amino) benzamide formate | 616.0 | 616.3 (M + H)+ | in analogy to the procedure for example 11: coupling of N-(2-aminoethyl)-2-chloro- 4-((3-(1-(cyano methyl)-3- (trifluoromethyl)-1H- pyrazol-4-yl)imidazo[1,2- a]pyrazin-8- yl)amino)benzamide formate with tert-butyl 3-(2- chloroacetamido)azetidine- 1-carboxylate and subsequent acidic Boc deprotection |
| 817 | 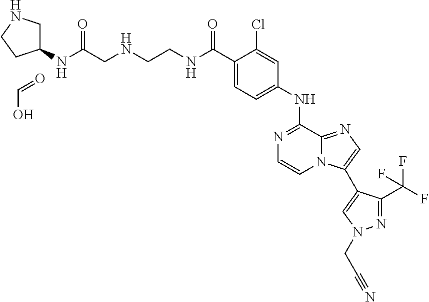 | (S)-2-chloro-4- ((3-(1- (cyanomethyl)-3- (trifluoromethyl)- 1H-pyrazol-4- yl)imidazo[1,2- a]pyrazin-8- yl)amino)-N-(2- ((2-oxo-2- (pyrrolidin-3- ylamino)ethyl) amino)ethyl) benzamide formate | 630.0 | 630.3 (M + H)+ | in analogy to the procedure for example 11: coupling of N-(2-aminoethyl)-2-chloro- 4-((3-(1-(cyanomethyl)-3- (trifluoromethyl)-1H- pyrazol-4-yl)imidazo[1,2- a]pyrazin-8- yl)amino)benzamide formate with tert-butyl (S)- 3-(2- chloroacetamido)pyrrolidine- 1-carboxylate and subsequent acidic Boc deprotection |
| 818 | 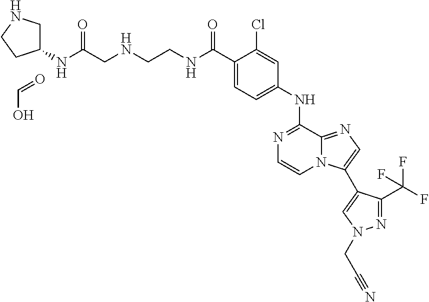 | (R)-2-chloro-4- ((3-(1- (cyanomethyl)-3- (trifluoromethyl)- 1H-pyrazol-4- yl)imidazo[1,2- a]pyrazin-8- yl)amino)-N-(2- ((2-oxo-2- (pyrrolidin-3- ylamino)ethyl) amino)ethyl) benzamide formate | 630.0 | 630.3 (M + H)+ | in analogy to the procedure for example 11: coupling of N-(2-aminoethyl)-2-chloro- 4-((3-(1-(cyanomethyl)-3- (trifluoromethyl)-1H- pyrazol-4-yl)imidazo[1,2- a]pyrazin-8- yl)amino)benzamide formate with tert-butyl (R)- 3-(2- chloroacetamido)pyrrolidine- 1-carboxylate and subsequent acidic Boc deprotection |
| 819 | 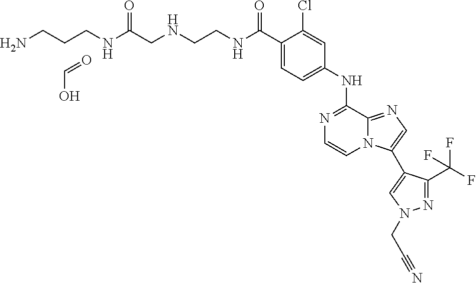 | N-(2-((2-((3- aminopropyl) amino)-2- oxoethyl)amino) ethyl)-2-chloro-4- ((3-(1- (cyanomethyl)-3- (trifluoromethyl)- 1H-pyrazol-4- yl)imidazo[1,2- a]pyrazin-8- yl)amino) benzamide formate | 618.0 | 618.3 (M + H)+ | in analogy to the procedure for example 11: coupling of N-(2-aminoethyl)-2-chloro- 4-((3-(1-(cyanomethyl)-3- (trifluoromethyl)-1H- pyrazol-4-yl)imidazo[1,2- a]pyrazin-8- yl)amino)benzamide formate with tert-butyl (3- (2- chloroacetamido)propyl) carbamate and subsequent acidic Boc deprotection |
| 820 | 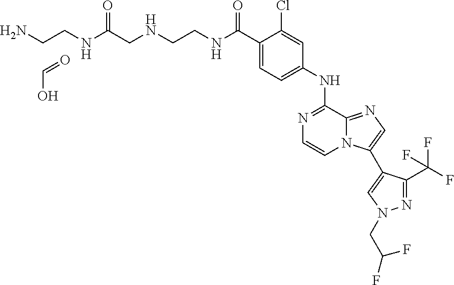 | N-(2-((2-((2- aminoethyl) amino)-2- oxoethyl)amino) ethyl)-2-chloro-4- (3-(1-(2,2- difluoroethyl)-3- (trifluoromethyl)- 1H-pyrazol-4- yl)imidazo[1,2- a]pyrazin-8- yl)amino) benzamide formate | 628.9 | 629.2 (M + H)+ | in analogy to the procedure for example 11: alkylation of tert-butyl (2-(2-chloro-4- ((3-(3-(trifluoromethyl)-1H- pyrazol-4-yl)imidazo[1,2- a]pyrazin-8- yl)amino)benzamido)ethyl) carbamate formate with 1,1- difluoro-2-iodoethane, acidic Boc deprotection, coupling with tert-butyl (2- (2- chloroacetamido)ethyl) carbamate and subsequent acidic Boc deprotection |
| 821 | 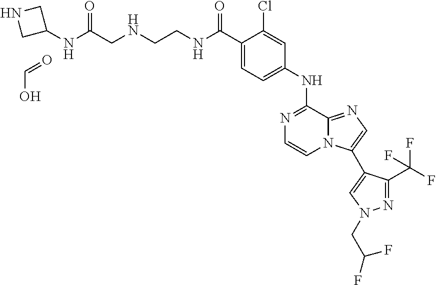 | N-(2-((2- (azetidin-3- ylamino)-2- oxoethyl)amino) ethyl)-2-chloro-4- (3-(1-(2,2- difluoroethyl)-3- (trifluoromethyl)- 1H-pyrazol-4- yl)imidazo[1,2- a]pyrazin-8- yl)amino) benzamide formate | 641.0 | 641.3 (M + H)+ | in analogy to the procedure for example 11: alkylation of tert-butyl (2-(2-chloro-4- ((3-(3-(trifluoromethyl)-1H- pyrazol-4-yl)imidazo[1,2- a]pyrazin-8- yl)amino)benzamido)ethyl) carbamate formate with 1,1- difluoro-2-iodoethane, acidic Boc deprotection, coupling with tert-butyl 3- (2- chloroacetamido)azetidine- 1-carboxylate and subsequent acidic Boc deprotection |
| 822 | 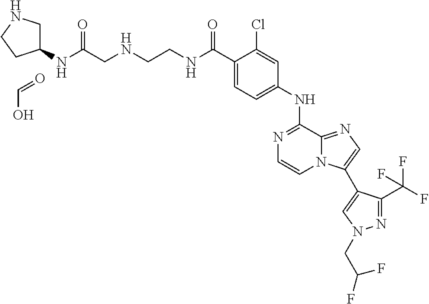 | (S)-2-chloro-4- (3-(1-2,2- difluoroethyl)-3- (trifluoromethyl)- 1H-pyrazol-4- yl)imidazo[1,2- a]pyrazin-8- yl)amino)-N-(2- ((2-oxo-2- (pyrrolidin-3- ylamino)ethyl) amino)ethyl) benzamide formate | 655.0 | 655.3 (M + H)+ | in analogy to the procedure for example 11: alkylation of tert-butyl (2-(2-chloro-4- ((3-(3-(trifluoromethyl)-1H- pyrazol-4-yl)imidazo[1,2- a]pyrazin-8- yl)amino)benzamido)ethyl) carbamate formate with 1,1- difluoro-2-iodoethane, acidic Boc deprotection, coupling with tert-butyl (S)- 3-(2- chloroacetamido)pyrrolidine- 1-carboxylate and subsequent acidic Boc deprotection |
| 823 | 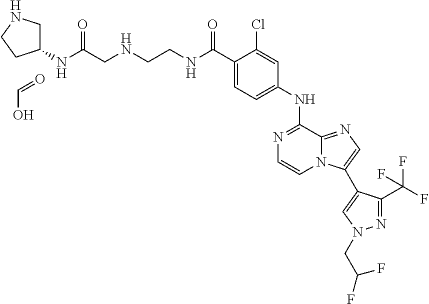 | (R)-2-chloro-4- (3-(1-(2,2- difluoroethyl)-3- (trifluoromethyl)- 1H-pyrazol-4- yl)imidazo[1,2- a]pyrazin-8- yl)amino)-N-(2- ((2-oxo-2- (pyrrolidin-3- ylamino)ethyl) amino)ethyl) benzamide formate | 655.0 | 655.3 (M + H)+ | in analogy to the procedure for example 11: alkylation of tert-butyl (2-(2-chloro-4- ((3-(3-(trifluoromethyl)-1H- pyrazol-4-yl)imidazo[1,2- a]pyrazin-8- yl)amino)benzamido)ethyl) carbamate formate with 1,1- difluoro-2-iodoethane, acidic Boc deprotection, coupling with tert-butyl (R)- 3-(2- chloroacetamido)pyrrolidine- 1-carboxylate and subsequent acidic Boc deprotection |
| 824 | 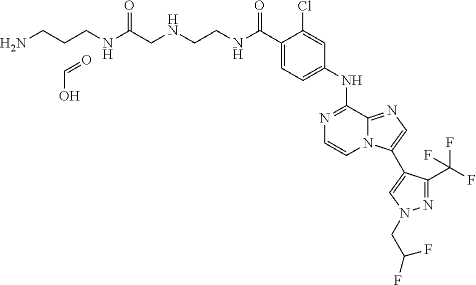 | N-(2-((2-((3- aminopropyl) amino)-2- oxoethyl)amino) ethyl)-2-chloro-4- (3-(1-2,2- difluoroethyl)-3- (trifluoromethyl)- 1H-pyrazol-4- yl)imidazo[1,2- a]pyrazin-8- yl)amino) benzamide formate | 643.0 | 643.3 (M + H)+ | in analogy to the procedure for example 11: alkylation of tert-butyl (2-(2-chloro-4- ((3-(3-(trifluoromethyl)-1H- pyrazol-4-yl)imidazo[1,2- a]pyrazin-8- yl)amino)benzamido)ethyl) carbamate formate with 1,1- difluoro-2-iodoethane, acidic Boc deprotection, coupling with tert-butyl (3- (2- chloroacetamido)propyl) carbamate and subsequent acidic Boc deprotection |

### Example 825

### (1s,5r)-6-[[2-chloro-4-[[3-[1-(cyanomethyl)-3-(trifluoromethyl)pyrazol-4-yl]imidazo[1,2-α]pyrazin-8-yl]amino]benzoyl]amino]-N-[(3R,4R)-4 hydroxypyrrolidin-3-yl]-3-azabicyclo[3.1.0]hexane-3-carboxamide formic acid salt

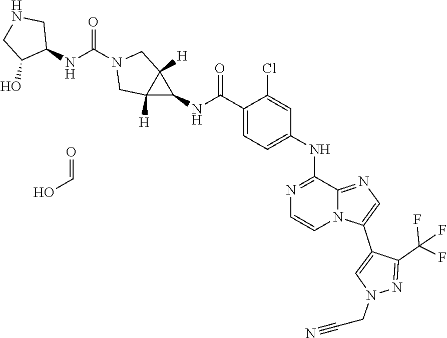 [SMILES: N#CCn1cc(-c2cnc3c(Nc4ccc(C(=O)N[C@H]5[C@@H]6CN(C(=O)N[C@@H]7CNC[C@H]7O)C[C@@H]65)c(Cl)c4)nccn23)c(C(F)(F)F)n1 ; O=CO]

Step 1:

### (1s,5r)-6-[[2-chloro-4-[[3-[3-(trifluoromethyl)-1H-pyrazol-4-yl]imidazo[1,2-α]pyrazin-8-yl]amino]benzoyl]amino]-3-azabicyclo[3.1.0]hexane-3-carboxylic acid tert-butyl ester

2-chloro-4-[[3-[3-(trifluoromethyl)-1H-pyrazol-4-yl]imidazo[1,2-α]pyrazin-8-yl]amino]benzoic acid (470.36 mg, 1.06 mmol, 1 eq) was combined with (1s,5r)-6-amino-3-azabicyclo[3.1.0]hexane-3-carboxylic acid tert-butyl ester (262.5 mg, 1.32 mmol, 1.25 eq), HATU (483.3 mg, 1.27 mmol, 1.2 eq) and DIPEA (684.5 mg, 925. uL, 5.3 mmol, 5 eq) in DMF (10 mL) at room temperature for 2.5 hr. The reaction mixture was poured into 100 mL brine and extracted with EtOAc (2×75 mL). The organic layers were combined, dried with sodium sulfate, and purified by flash chromatography to afford to afford the title compound (248 mg, 38.8%) as off-white solid. MS(m/e): 601.4 (M−H).

Step 2:

### N-[(1s,5r)-3-azabicyclo[3.1.0]hexan-6-yl]-2-chloro-4-[[3-[3-(trifluoromethyl)-1H-pyrazol-4-yl]imidazo[1,2-α]pyrazin-8-yl]amino]benzamide hydrogen chloride

(1s,5r)-6-[[2-chloro-4-[[3-[3-(trifluoromethyl)-1H-pyrazol-4-yl]imidazo[1,2-α]pyrazin-8-yl]amino]benzoyl]amino]-3-azabicyclo[3.1.0]hexane-3-carboxylic acid tert-butyl ester (251.7 mg, 0.418 mmol, 1 eq) dissolved in DCM (5 mL) was treated with an excess of 4 M HCl in Dioxane (3.76 g, 3.13 mL, 12.53 mmol, 30 eq) over night at room temperature. The mixture was concentrated in vacuo and dried to afford the crude title compound (296 mg, 99.9%) as white solid. MS(m/e): 501.4 (M−H).

Step 3:

### (3R,4R)-3-[[(1s,5r)-6-[[2-chloro-4-[[3-[3-(trifluoromethyl)-1H-pyrazol-4-yl]imidazo[1,2-α]pyrazin-8-yl]amino]benzoyl]amino]-3-azabicyclo[3.1.0]hexane-3-carbonyl]amino]-4-hydroxy-pyrrolidine-1-carboxylic acid tert-butyl ester

tert-butyl-3R-amino-4R-hydroxypyrrolidine-1-carboxylate (134.98 mg, 0.667 mmol, 1.6 eq) was combined in DMF (10 mL), TEA (337.66 mg, 465.1 uL, 3.34 mmol, 8 eq) and CDI (108.21 mg, 0.667 mmol, 1.6 eq) at room temperature for 10 min. N-[(1s,5r)-3-azabicyclo[3.1.0]hexan-6-yl]-2-chloro-4-[[3-[3-(trifluoromethyl)-1H-pyrazol-4-yl]imidazo[1,2-α]pyrazin-8-yl]amino]benzamide hydrogen chloride (224.96 mg, 0.417 mmol, 1 eq) was added and the mixture was stirred at room temperature for 2 hr. The reacure mixture was then poured into water and extracted with AcOEt.

The organic layers were combined, dried with sodium sulfate and purified by flash chromatography to afford to afford the title compound (286.6 mg, 92.1%) as white solid. MS(m/e): 729.9 (M−H).

Step 4:

### (3R,4R)-3-[[(1s,5r)-6-[[2-chloro-4-[[3-[1-(cyanomethyl)-3-(trifluoromethyl)pyrazol-4-yl]imidazo[1,2-α]pyrazin-8-yl]amino]benzoyl]amino]-3-azabicyclo[3.1.0]hexane-3-carbonyl]amino]-4-hydroxy-pyrrolidine-1-carboxylic acid tert-butyl ester

To (3R,4R)-3-[[(1s,5r)-6-[[2-chloro-4-[[3-[3-(trifluoromethyl)-1H-pyrazol-4-yl]imidazo[1,2-α]pyrazin-8-yl]amino]benzoyl]amino]-3-azabicyclo[3.1.0]hexane-3-carbonyl]amino]-4-hydroxy-pyrrolidine-1-carboxylic acid tert-butyl ester (286.6 mg, 0.384 mmol, 1 eq) dissolved N,N-dimethylformamide (3 mL) was added potassium carbonate (265.4 mg, 1.92 mmol, 5 eq) followed by 2-bromoacetonitrile (115.19 mg, 0.960 mmol, 2.5 eq). The mixture was stirred at room temperature for 3 hr. The reaction mixture was then poured into water and the product was extracted with EtOAc.

The organic layers were combined, dried over sodium sulfate and concentrated in vacuo to afford the crude title compound (215.4 mg, 72.8%) as a light brown solid. MS(m/e): 770.5 (M+H).

Step 5:

### (1s,5r)-6-[[2-chloro-4-[[3-[1-(cyanomethyl)-3-(trifluoromethyl)pyrazol-4-yl]imidazo[1,2-α]pyrazin-8-yl]amino]benzoyl]amino]-N-[(3R,4R)-4-hydroxypyrrolidin-3-yl]-3-azabicyclo[3.1.0]hexane-3-carboxamide formic acid salt

To (3R,4R)-3-[[(1s,5r)-6-[[2-chloro-4-[[3-[1-(cyanomethyl)-3-(trifluoromethyl)pyrazol-4-yl]imidazo[1,2-α]pyrazin-8-yl]amino]benzoyl]amino]-3-azabicyclo[3.1.0]hexane-3-carbonyl]amino]-4-hydroxy-pyrrolidine-1-carboxylic acid tert-butyl ester (215.4 mg, 0.280 mmol, 1 eq) dissolved in dichloromethane (8 mL) was treated with an excess of TFA (318.89 mg, 215.47 uL, 2.8 mmol, 10 eq) at room temperature for 2 hr. The mixture was cautionary quenched with TEA and purified by preparative HPLC to afford the title compound (129 mg, 64.4%) as light brown solid. MS(m/e): 670.4 (M+H).

### Example 826

### N-(6-aminohexyl)-2-chloro-4-((3-(1-(2,2-difluoroethyl)-3-(trifluoromethyl)-1H-pyrazol-4-yl)imidazo[1,2-α]pyrazin-8-yl)amino)benzamide hydrochloride

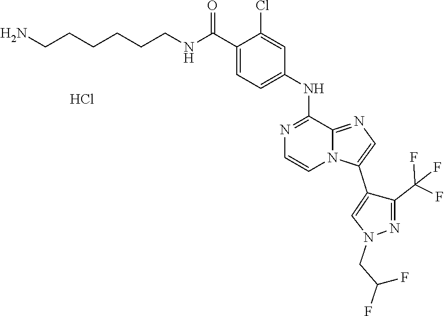 [SMILES: NCCCCCCNC(=O)c1ccc(Nc2nccn3c(-c4cn(CC(F)F)nc4C(F)(F)F)cnc23)cc1Cl ; Cl]

To 2-chloro-4-((3-(1-(2,2-difluoroethyl)-3-(trifluoromethyl)-1H-pyrazol-4-yl)imidazo[1,2-α]pyrazin-8-yl)amino)benzoic acid (190 mg, 0.363 mmol, 1 eq), dissolved in DMF (3 ml) was added DIPEA (143.62 mg, 194.08 uL, 1.09 mmol, 3 eq), HATU (170.75 mg, 0.436 mmol, 1.2 eq) and tert-butyl (6-aminohexyl)carbamate hydrochloride (100.9 mg, 0.399 mmol, 1.1 eq). The mixture was stirred at room temperature over night. The reaction mixture was poured into water and the product was extracted with AcOEt. The organic layers were combined, dried with magnesium sulfate, filtered and dried in vacuo to afford the crude N-Boc protected intermediate (240 mg, 96.5%) as light brown solid, which is dissolved in 2 ml of DCM and treated with an excess of 4M HCl in dioxane (2.63 ml, 10.5 mmol, Eq: 30) at room temperature overnight. The resulting solid was triturated with diethyl ether and collected by filtration to afford the title compound (153 mg, 241 μmol, 68.9% yield) as a white solid. MS(m/e): 585.1 (M+H).

### Example 827

### N-((1-(azetidin-3-ylmethyl)pyrrolidin-3-yl)methyl)-2-ethyl-4-((3-(3-(trifluoromethyl)-1H-pyrazol-4-yl)imidazo[1,2-α]pyrazin-8-yl)amino)benzamide bis(2,2,2-trifluoroacetate)

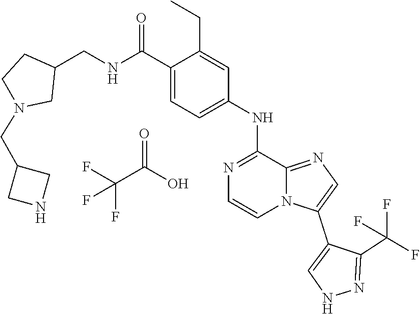

Step 1:

### tert-butyl 3-((2-ethyl-4-((3-iodoimidazo[1,2-α]pyrazin-8-yl)amino)benzamido)methyl)pyrrolidine-1-carboxylate

2-ethyl-4-((3-iodoimidazo[1,2-α]pyrazin-8-yl)amino)benzoic acid (150 mg, 367 μmol, Eq: 1), tert-butyl 3-(aminomethyl)pyrrolidine-1-carboxylate (95.7 mg, 478 μmol, Eq: 1.3), HATU (182 mg, 478 μmol, Eq: 1.3) and DIPEA (142 mg, 193 μl, 1.1 mmol, Eq: 3) were combined with DMF (5 ml). The reaction was stirred at room temperature for 30 min. The reaction mixture was poured into 25 mL water and extracted with EtOAc. The organic layers were combined, dried with sodium sulfate and purified by flash chromatography to afford the title compound (165 mg, 76%). MS(m/e): 591.3 (M+H).

Step 2:

### tert-butyl 3-((2-ethyl-4-((3-(3-(trifluoromethyl)-1-((2-(trimethylsilyl)ethoxy)methyl)-1H-pyrazol-4-yl)imidazo[1,2-α]pyrazin-8-yl)amino)benzamido)methyl)pyrrolidine-1-carboxylate

tert-butyl 3-((2-ethyl-4-((3-iodoimidazo[1,2-α]pyrazin-8-yl)amino)benzamido)methyl)-pyrrolidine-1-carboxylate (120 mg, 203 μmol, Eq: 1), 4-(4,4,5,5-tetramethyl-1,3,2-dioxaborolan-2-yl)-3-(trifluoromethyl)-1-((2-(trimethylsilyl)ethoxy)methyl)-1H-pyrazole (120 mg, 305 μmol, Eq: 1.5), 1,1′-bis(di-tert-butylphosphino)ferrocene palladium dichloride (13.2 mg, 20.3 μmol, Eq: 0.1) and Na₂CO₃ (64.6 mg, 610 μmol, Eq: 3) were heated under microwave irradiations in a mix of dioxane (3 ml)/water (0.3 ml) for 2 hr. The crude reaction mixture was concentrated in vacuo and the crude material was purified by flash chromatography to afford the title compound (120 mg, 81%). MS(m/e): 729.6 (M+H).

Step 3:

### 2-ethyl-N-(pyrrolidin-3-ylmethyl)-4-((3-(3-(trifluoromethyl)-1H-pyrazol-4-yl)imidazo[1,2-α]pyrazin-8-yl)amino)benzamide

tert-butyl 3-((2-ethyl-4-((3-(3-(trifluoromethyl)-1-((2-(trimethylsilyl)ethoxy)methyl)-1H-pyrazol-4-yl)imidazo[1,2-α]pyrazin-8-yl)amino)benzamido)methyl)pyrrolidine-1-carboxylate (120 mg, 165 μmol, Eq: 1) in THF (3 ml) was treated with an excess of aquous HCl (37% in water) (1.37 ml, 16.5 mmol, Eq: 100) at room temperature for 1 hr. The crude reaction mixture was concentrated in vacuo to afford the title compound which was used crude for next step. MS(m/e): 499.3 (M+H).

Step 4:

### tert-butyl 3-((3-((2-ethyl-4-((3-(3-(trifluoromethyl)-1H-pyrazol-4-yl)imidazo[1,2-α]pyrazin-8-yl)amino)benzamido)methyl)pyrrolidin-1-yl)methyl)azetidine-1-carboxylate

tert-butyl 3-formylazetidine-1-carboxylate (91.4 mg, 493 μmol, Eq: 3), crude 2-ethyl-N-(pyrrolidin-3-ylmethyl)-4-((3-(3-(trifluoromethyl)-1H-pyrazol-4-yl)imidazo[1,2-α]pyrazin-8-yl)amino)benzamide (Eq: 1) and NaBH₃CN (51.7 mg, 822 μmol, Eq: 5) were combined in MeOH (6 ml) at room temperature for 15 hr. The crude reaction mixture was concentrated in vacuo. The reaction mixture was poured into 25 mL water and extracted with EtOAc. The organic layers were combined, dried over Na₂SO₄ and concentrated in vacuo to afford the title compound (110 mg, 100%). MS(m/e): 668.5 (M+H).

Step 5:

### N-((1-(azetidin-3-ylmethyl)pyrrolidin-3-yl)methyl)-2-ethyl-4-((3-(3-(trfluoromethyl)-1H-pyrazol-4-yl)imidazo[1,2-α]pyrazin-8-yl)amino)benzamide bis(2,2,2-trifluoroacetate)

tert-butyl 3-((3-((2-ethyl-4-((3-(3-(trifluoromethyl)-1H-pyrazol-4-yl)imidazo[1,2-α]pyrazin-8-yl)amino)benzamido)methyl)pyrrolidin-1-yl)methyl)azetidine-1-carboxylate (27 mg, 40.4 μmol, Eq: 1) dissolved in DCM (2 ml) was treated with an excess of TFA (922 mg, 8.09 mmol, Eq: 200) at room temperature for 1 hr. The crude material was purified by preparative HPLC to afford the title compound (13 mg, 16 μmol, 39.6%). MS(m/e): 568.2 (M+H).

### Example 828

### N-((1-(azetidin-3-ylmethyl)piperidin-4-yl)methyl)-4-((3-(1-(cyanomethyl)-3-(trifluoromethyl)-1H-pyrazol-4-yl)imidazo[1,2-α]pyrazin-8-yl)amino)-2-ethylbenzamide 2,2,2-trifluoroacetate

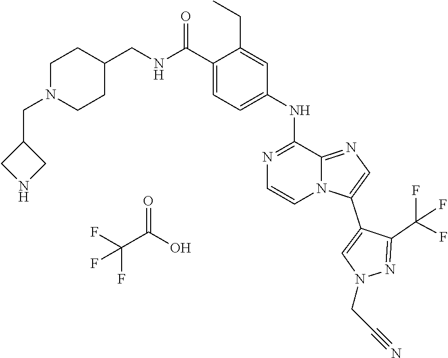 [SMILES: CCc1cc(Nc2nccn3c(-c4cn(CC#N)nc4C(F)(F)F)cnc23)ccc1C(=O)NCC1CCN(CC2CNC2)CC1 ; O=C(O)C(F)(F)F]

Step 1:

### tert-butyl 4-((2-ethyl-4-((3-iodoimidazo[1,2-α]pyrazin-8-yl)amino)benzamido)methyl)piperidine-1-carboxylate

To 2-ethyl-4-((3-iodoimidazo[1,2-α]pyrazin-8-yl)amino)benzoic acid (2.0 g, 5 mmol, Eq: 1) and tert-butyl 4-(aminomethyl)piperidine-1-carboxylate (1.3 g, 6 mmol, Eq: 1.2) in DMF (20 mL) was added DIPEA (1.54 g, 12 mmol, Eq: 2.4). The resultant mixture was stirred for 10 min at room temperature, HATU (4.6 g, 12 mmol, Eq: 2.4) was added and the mixture was stirred for 4.0 hr at room temperature. The mixture was poured into water and was extracted with DCM. The organic layers were combined and washed with water and brine, dried over anhydrous Na₂SO₄ and concentrated under reduced pressure to give the crude title compound (2.6 g, 86%) which was used without purification. MS(m/e): 605.0 (M+H).

Step 2:

### 2-ethyl-4-((3-iodoimidazo[1,2-α]pyrazin-8-yl)amino)-N-(piperidin-4-ylmethyl)benzamide

A solution of tert-butyl 4-((2-ethyl-4-((3-iodoimidazo[1,2-α]pyrazin-8-yl)amino)benzamido)methyl)piperidine-1-carboxylate (2.6 g, 4.3 mmol, Eq: 1) in DCM (40 ml) was treated with an excess of TFA (5.0 mL) at room temperature for 5 hr and then adjusted to pH=7-8 with aqueous ammonia. The mixture was poured into water and then extracted with dichloromethane/isopropanol (100/10 mL), the organic layer was concentrated in vacuo to afford the title compound (2.0 g, 92%) which was used without purification. MS(m/e): 505.1 (M+H).

Step 3:

### tert-butyl 3-((4-((2-ethyl-4-((3-iodoimidazo[1,2-α]pyrazin-8-yl)amino)benzamido)methyl)-piperidin-1-yl)methyl)azetidine-1-carboxylate

N-(3-chloro-4-((piperidin-4-ylmethyl)carbamoyl)phenyl)-5-(2,3-difluoro-4-methoxyphenyl)-1-methyl-1H-imidazole-2-carboxamide (1.0 g, 2.0 mmol, Eq: 1), tert-butyl 3-formylpyrrolidine-1-carboxylate (740 mg, 4.0 mmol, Eq: 2.0) and sodium cyanoborohydride (667 mg, 8 mmol, Eq: 4) were combined with MeOH (10 ml) for 4.0 hr at room temperature. The mixture was poured into water and then extracted with dichloromethane. The organic layers were combined and washed with water and brine, dried over anhydrous Na₂SO₄ and purified by silica column to afford the title compound (1.2 g, 89%). MS(m/e): 674.4 (M+H).

Step 4:

### 2-(4-(4,4,5,5-tetramethyl-1,3,2-dioxaborolan-2-yl)-3-(trifluoromethyl)-1H-pyrazol-1-yl)acetonitrile

To a solution of 4-(4,4,5,5-tetramethyl-1,3,2-dioxaborolan-2-yl)-3-(trifluoromethyl)-1H-pyrazole (260 mg, 1.0 mmol, Eq: 1) and DIPEA (258 mg, 2.0 mmol, Eq: 2) in DCM (10 mL) was added 2-bromoacetonitrile (144 mg, 1.2 mmol, Eq: 1.2) and then stirred at room temperature for 4 hr. The mixture was poured into water and then extracted with dichloromethane, the organic layer was concentrated and purified by silica column to afford the title compound (250 mg, 83%). MS(m/e): 302.1 (M+H).

Step 5:

### tert-butyl 3-((4-((4-((3-(1-(cyanomethyl)-3-(trifluoromethyl)-1H-pyrazol-4-yl)imidazo[1,2-α]pyrazin-8-yl)amino)-2-ethylbenzamido)methyl)piperidin-1-yl)methyl)azetidine-1-carboxylate

A mixture of tert-butyl 3-((4-((2-ethyl-4-((3-iodoimidazo[1,2-α]pyrazin-8-yl)amino)benzamido)methyl)piperidin-1-yl)methyl)azetidine-1-carboxylate (340 mg, 0.5 mmol, Eq: 1), 2-(4-(4,4,5,5-tetramethyl-1,3,2-dioxaborolan-2-yl)-3-(trifluoromethyl)-1H-pyrazol-1-yl)acetonitrile (181 mg, 0.6 mmol, Eq: 1.2), Na₂CO₃ (159 mg, 1.5 mmol, Eq: 3) and 1,1′-bis(diphenylphosphino)ferrocene-palladium(II)dichloride dichloromethane complex (33 mg, 0.05 mmol, Eq: 0.1) in 1,4-Dioxane (20 ml) and water (2.0 ml) was stirred at 100° C. for 4 hr. The mixture was poured into water and extracted with DCM. The organic layers were combined, washed with water and brine, dried over anhydrous Na₂SO₄ and purified by silica column to afford the title compound (285 mg, 79%). MS(m/e): 721.3 (M+H).

Step 6:

### N-((1-(azetidin-3-ylmethyl)piperidin-4-yl)methyl)-4-((3-(1-(cyanomethyl)-3-(trifluoromethyl)-1H-pyrazol-4-yl)imidazo[1,2-α]pyrazin-8-yl)amino)-2-ethylbenzamide 2,2,2-trifluoroacetate

tert-butyl 3-((4-((4-((3-(1-(cyanomethyl)-3-(trifluoromethyl)-1H-pyrazol-4-yl)imidazo[1,2-α]pyrazin-8-yl)amino)-2-ethylbenzamido)methyl)piperidin-1-yl)methyl)azetidine-1-carboxylate (280 mg, 2.4 mmol, Eq: 1) in THF (10 mL) was treated with an excess of TFA (3.0 mL) for 4.0 h at room temperature. The pH of the mixture was adjusted to 8-9 with aqueous ammonia. The mixture was poured into water and then extracted with dichloromethane/isopropanol (100/10 mL), the organic layer was concentrated in vacuo and purified by preparative HPLC to afford the title compound (16 mg, 5.2%). MS(m/e): 621.3 (M+H).

### Example 829

### 2-[azetidin-3-ylmethyl-[S-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]amino]acetic acid; formic acid

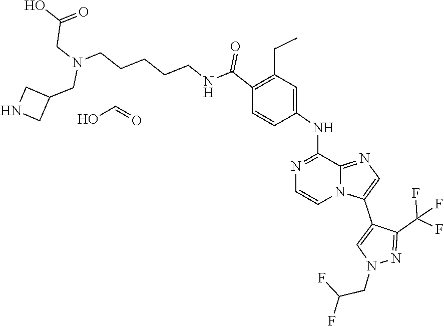 [SMILES: CCc1cc(Nc2nccn3c(-c4cn(CC(F)F)nc4C(F)(F)F)cnc23)ccc1C(=O)NCCCCCN(CC(=O)O)CC1CNC1 ; O=CO]

Step 1:

### tert-butyl N-[5-[(4-nitro-2-vinyl-benzoyl)amino]pentyl]carbamate

A mixture of 4-nitro-2-vinyl-benzoic acid (4.3 g, 22.26 mmol, 1 eq), 4-nitro-2-vinyl-benzoic acid (4.3 g, 22.26 mmol, 1 eq), triethylamine (9.31 mL, 66.78 mmol, 3 eq) and propylphonic anhydride (19.86 mL, 33.39 mmol, 1.5 eq) in THF (50 mL) was stirred at room temperature for 16 h. The mixture was poured into water, extracted with EtOAc, washed with brine, dried over sodium sulfate. The crude mixture was purified by HPLC preparative to afford the title compound (6 g, 15.9 mmol, 71.4%). MS(m/e): 400.2 (M+Na).

Step 2:

### tert-butyl N-[5-[(4-amino-2-ethyl-benzoyl)amino]pentyl]carbamate

tert-butyl N-[5-[(4-nitro-2-vinyl-benzoyl)amino]pentyl]carbamate (9.0 g, 23.85 mmol, 1 eq) and Pd/C (1.24 mL, 1.19 mmol, 0.050 eq) in methanol (90 mL) were stirred under H₂ atmosphere at room temperature for 16 h. The mixture was poured into water and extracted with EtOAc, washed with brine, dried over sodium sulfate and purified by silica column to afford the title compound (7.9 g, 22.61 mmol, 94.8% yield) as light yellow oil. MS(m/e): 350.2 (M+H).

Step 3:

### 8-chloro-3-[1-(2,2-difluoroethyl)-3-(trifluoromethyl)pyrazol-4-yl]imidazo[1,2-α]pyrazine

To a mixture of 8-chloro-3-[3-(trifluoromethyl)-1H-pyrazol-4-yl]imidazo[1,2-α]pyrazine (1.5 g, 5.22 mmol, 1 eq) and potassium carbonate (1801 mg, 13.0 mmol, 2.5 eq) in ACN (30 mL) was added 2,2-difluoroethyl trifluoromethanesulfonate (1.34 g, 6.26 mmol, 1.2 eq) at −10° C. The reaction was stirred at 0° C. for 16 hr. The mixture was poured into water, extracted with EtOAc and purified by silica column to afford the title compound (760 mg, 41% yield). MS(m/e): 352.1 (M+H).

Step 4:

### tert-butyl N-[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]carbamate

A mixture of tert-butyl N-[5-[(4-amino-2-ethyl-benzoyl)amino]pentyl]carbamate (496.8 mg, 1.42 mmol, 1 eq) and 8-chloro-3-[1-(2,2-difluoroethyl)-3-(trifluoromethyl)pyrazol-4-yl]imidazo[1,2-α]pyrazine (1.24 mL, 1.42 mmol, 1 eq) in a mix of ACN (8 mL) and acetic acid (0.500 mL) was stirred at 60° C. for 16 hr. The mixture was purified by HPLC preparative to afford the title compound (640 mg, 0.960 mmol, 68% yield). MS(m/e): 665.3 (M+H).

Step 5:

### N-(5-aminopentyl)-4-[[3-[1-(2,2-difluoroethyl)-3-(trifluoromethyl)pyrazol-4-yl]imidazo[1,2-α]pyrazin-8-yl]amino]-2-ethyl-benzamide

tert-butyl N-[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]carbamate (640.0 mg, 0.96 mmol, 1 eq) in DCM (5.6 mL) was treated with an excess of TFA (0.58 mL, 7.54 mmol, 7.83 eq) at 20° C. for 16 hr. The mixture was purified by HPLC preparative to afford the title compound (420 mg, 0.74 mmol, 77% yield). MS(m/e): 565.3 (M+H).

Step 6:

### tert-butyl 3-[[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentylamino]methyl]azetidine-1-carboxylate

To a mixture of N-(5-aminopentyl)-4-[[3-[1-(2,2-difluoroethyl)-3-(trifluoromethyl)pyrazol-4-yl]imidazo[1,2-α]pyrazin-8-yl]amino]-2-ethyl-benzamide (290.0 mg, 0.51 mmol, 1 eq), tert-butyl 3-formylazetidine-1-carboxylate (0.06 mL, 0.62 mmol, 1.2 eq) in methanol (3 mL) was added sodium triacetoxyborohydride (217.74 mg, 1.03 mmol, 2 eq) and the mixture was stirred at 40° C. for 16 hr. The reaction was purified by silica column to afford the title compound (240 mg, 0.330 mmol, 64% yield). MS(m/e): 734.3 (M+H).

Step 7:

### tert-butyl 3-[[(2-tert-butoxy-2-oxo-ethyl)-[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]amino]methyl]azetidine-1-carboxylate

To a mixture of tert-butyl 3-[[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentylamino]methyl]azetidine-1-carboxylate (45.0 mg, 0.06 mmol, 1 eq) and triethylamine (0.02 mL, 0.12 mmol, 2 eq) in ACN (2.35 mL) was added tert-butyl bromoacetate (0.04 mL, 0.310 mmol, 5 eq). The mixture was stirred at 20° C. for 16 hr. The mixture was purified by HPLC preparative to afford the title compound (15 mg, 0.02 mmol, 29% yield). MS(m/e): 848.4 (M+H).

Step 8:

### 2-[azetidin-3-ylmethyl-[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]amino]acetic acid; formic acid

tert-butyl 3-[[(2-tert-butoxy-2-oxo-ethyl)-[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]amino]methyl]azetidine-1-carboxylate (15.0 mg, 0.020 mmol, 1 eq) in DCM (1 mL) was treated with an excess of TFA (0.5 mL, 6.49 mmol, 366 eq) and stirred at room temperature for 5 hr. The mixture was purified by HPLC preparative to afford the title compound (6.7 mg, 0.01 mmol, 49% yield). MS(m/e): 692.3 (M+H).

### Example 830

### N-(2-(2-aminoethoxy)ethyl)-4-((3-(3-cyano-1-(cyanomethyl)-1H-pyrazol-4-yl)imidazo[1,2-α]pyrazin-8-yl)amino)-2-ethyl-benzamide

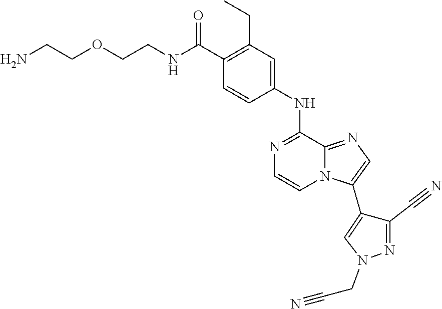 [SMILES: CCc1cc(Nc2nccn3c(-c4cn(CC#N)nc4C#N)cnc23)ccc1C(=O)NCCOCCN]

Step 1:

### methyl 4-iodo-1-trityl-1H-pyrazole-3-carboxylate

A solution of methyl 4-iodo-1H-pyrazole-3-carboxylate (8.2 g, 32.54 mmol, 1 eq) in THF (80 mL) was added sodium hydride, 60% in oil (1.43 g, 35.79 mmol, 1.1 eq) at 0° C. and stirred for 0.5 h, then triphenylmethyl chloride (9.98 g, 35.79 mmol, 1.1 eq) was added to the solution in positions at 0° C. The mixture was warmed to 10° C. and stirred for 16 h. The mixture was quenched with sat.NH4Cl and extracted with EtOAc, the combined organic phase was washed with brine (200 mL), dried over sodium sulfate, filtered and concentrated in vacuum to give crude product, which was purified by silica gel column chromatography to give methyl 4-iodo-1-trityl-pyrazole-3-carboxylate (12.5 g, 25.29 mmol, 78% yield) as white solid. MS(m/e): 517.2 (M+Na).

Step 2:

### (3-(methoxycarbonyl)-1-trityl-1H-pyrazol-4-yl)boronic acid

To a solution of methyl 4-iodo-1-trityl-pyrazole-3-carboxylate (13.0 g, 26.3 mmol, 1 eq) and boron isopropoxide (9.1 mL, 39.45 mmol, 1.5 eq) in THF (100 mL) was added drop wise butyllithium solution (18.94 mL, 47.34 mmol, 1.8 eq) at −78° C. under N₂, then the mixture was stirred for 1 h at −78° C. The mixture was poured into NH₄Cl solution and extracted with EtOAc, the combined organic phase was washed by brine, dried over Na₂SO₄, concentrated to afford crude (3-methoxycarbonyl-1-trityl-pyrazol-4-yl)boronic acid (10 g, 24.26 mmol, 92% yield) as orange solid, which used for next step without purification. MS(m/e): 435.1 (M+Na).

Step 3:

### methyl 4-(8-chloroimidazo[1,2-α]pyrazin-3-yl)-1-trityl-1H-pyrazole-3-carboxylate

A mixture of 8-chloro-3-iodo-imidazo[1,2-α]pyrazine (5.0 g, 17.89 mmol, 1 eq), (3-methoxycarbonyl-1-trityl-pyrazol-4-yl)boronic acid (9.59 g, 23.26 mmol, 1.3 eq), [1,1′-bis(diphenylphosphino)ferrocene]dichloropalladium(II) (1309.11 mg, 1.79 mmol, 0.100 eq) and sodium carbonate (3792.46 mg, 35.78 mmol, 2 eq) in 1,4-dioxane (100 mL)/water (10 mL) was stirred under N2 at 85° C. for 16 h. The mixture was filtered, the filtrate was concentrated to give the crude product which was purified by silica column to afford methyl 4-(8-chloroimidazo[1,2-α]pyrazin-3-yl)-1-trityl-pyrazole-3-carboxylate (3.9 g, 7.5 mmol, 42% yield) as light yellow solid. MS(m/e): 520.1 (M+H).

Step 4:

### 4-(8-chloroimidazo[1,2-α]pyrazin-3-yl)-1-trityl-1H-pyrazole-3-carboxylic acid

To a solution of methyl 4-(8-chloroimidazo[1,2-α]pyrazin-3-yl)-1-trityl-pyrazole-3-carboxylate (3.9 g, 7.5 mmol, 1 eq) in THF (20 mL)/water (20 mL) was added hydroxylithium hydrate (1573.56 mg, 37.5 mmol, 5 eq), the reaction mixture was stirred at 15° C. for 16 h. The mixture was acidified to pH=6 with 1N HCl solution, then extracted with EtOAc, the combined organic phase was washed with brine and concentrated to afford 4-(8-chloroimidazo[1,2-α]pyrazin-3-yl)-1-trityl-pyrazole-3-carboxylic acid (3 g, 5.93 mmol, 79.06% yield) as light yellow solid. MS(m/e): 506.2 (M+H).

Step 5:

### 4-(8-chloroimidazo[1,2-α]pyrazin-3-yl)-1-trityl-1H-pyrazole-3-carboxamide

To a mixture of 4-(8-chloroimidazo[1,2-α]pyrazin-3-yl)-1-trityl-pyrazole-3-carboxylic acid (2.0 g, 3.95 mmol, 1 eq), ammonium chloride (4.23 g, 79.06 mmol, 20 eq) and triethylamine (1.1 mL, 7.91 mmol, 2 eq) in DMF (20 mL) was added 0-(7-azabenzotriazol-1-yl)-N,N,N′,N′-tetramethyluronium hexafluorophosphate (1803.64 mg, 4.74 mmol, 1.2 eq), the mixture was stirred at 15° C. for 16 h. The mixture was poured into water and extracted with EtOAc, the combined organic phase was concentrated to give the crude product which was purified by silica column to afford 4-(8-chloroimidazo[1,2-α]pyrazin-3-yl)-1-trityl-pyrazole-3-carboxamide (1.4 g, 2.77 mmol, 70.14% yield) as light yellow solid. MS(m/e): 505.3 (M+H).

Step 6:

### 4-(8-chloroimidazo[1,2-α]pyrazin-3-yl)-1-trityl-1H-pyrazole-3-carbonitrile

To a solution of 4-(8-chloroimidazo[1,2-α]pyrazin-3-yl)-1-trityl-pyrazole-3-carboxamide (800.0 mg, 1.58 mmol, 1 eq) in DCM (20 mL) was added burgess reagent (1132.58 mg, 4.75 mmol, 3 eq), the reaction mixture was stirred at 20° C. for 16 h. The mixture was concentrated to give a residue which was purified by silica column to afford 4-(8-chloroimidazo[1,2-α]pyrazin-3-yl)-1-trityl-pyrazole-3-carbonitrile (520 mg, 1.07 mmol, 67.41% yield) as white solid. MS(m/e): 487.2 (M+H).

Step 7:

### 4-(8-chloroimidazo[1,2-α]pyrazin-3-yl)-1H-pyrazole-3-carbonitrile

A solution of 4-(8-chloroimidazo[1,2-α]pyrazin-3-yl)-1-trityl-pyrazole-3-carbonitrile (420.0 mg, 0.860 mmol, 1 eq) in hydrochloric acid solution (10.5 mL, 42 mmol, 4 N in 1,4-dioxane) was stirred at 20° C. for 16 h. The mixture was concentrated and the obtained residue was dissolved in EtOAc (50 mL). The organic phase was washed with NaHCO₃ solution (50 mL), dried with Na₂SO₄ and concentrated to afford 4-(8-chloroimidazo[1,2-α]pyrazin-3-yl)-1H-pyrazole-3-carbonitrile (140 mg, 0.570 mmol, 66.35% yield) as light yellow solid. MS(m/e): 244.9 (M+H).

Step 8:

### tert-butyl (2-(2-(4-((3-(3-cyano-1H-pyrazol-4-yl)imidazo[1,2-α]pyrazin-8-yl)amino)-2-ethylbenzamido)ethoxy)ethyl)carbamate

A solution of 4-(8-chloroimidazo[1,2-α]pyrazin-3-yl)-1H-pyrazole-3-carbonitrile (120.6 mg, 0.490 mmol, 1.05 eq) and tert-butyl N-[2-[2-[(4-amino-2-ethyl-benzoyl)amino]ethoxy]ethyl]carbamate (165.0 mg, 0.470 mmol, 1 eq) in ACN (8 mL) and acetic acid (0.8 mL) was stirred at 70° C. for 16 h. The mixture was concentrated to afford a residue, which was purified by prep-TLC to afford tert-butyl N-[2-[2-[[4-[[3-(3-cyano-1H-pyrazol-4-yl)imidazo[1,2-α]pyrazin-8-yl]amino]-2-ethyl-benzoyl]amino]ethoxy]ethyl]carbamate (71 mg, 0.130 mmol, 27.02% yield) as white solid. MS(m/e): 560.2 (M+H).

Step 9:

### tert-butyl (2-(2-(4-((3-(3-cyano-1-(cyanomethyl)-1H-pyrazol-4-yl)imidazo[1,2-α]pyrazin-8-yl)amino)-2-ethylbenzamido)ethoxy)ethyl)carbamate

To a mixture of tert-butyl N-[2-[2-[[4-[[3-(3-cyano-1H-pyrazol-4-yl)imidazo[1,2-α]pyrazin-8-yl]amino]-2-ethyl-benzoyl]amino]ethoxy]ethyl]carbamate (70.0 mg, 0.130 mmol, 1 eq) and potassium carbonate (34.57 mg, 0.250 mmol, 2 eq) in ACN (5 mL) was added bromoacetonitrile (22.51 mg, 0.190 mmol, 1.5 eq) at 0° C., the mixture was stirred at 20° C. for 16 h. Then the mixture was filtered, the filtrate was concentrated and the obtained residue was purified by Prep-TLC to afford tert-butyl N-[2-[2-[[4-[[3-[3-cyano-1-(cyanomethyl)pyrazol-4-yl]imidazo[1,2-α]pyrazin-8-yl]amino]-2-ethyl-benzoyl]amino]ethoxy]ethyl]carbamate (60 mg, 0.100 mmol, 80% yield) as light yellow oil. MS(m/e): 599.4 (M+H).

Step 10:

### N-(2-(2-aminoethoxy)ethyl)-4-((3-(3-cyano-1-(cyanomethyl)-1H-pyrazol-4-yl)imidazo[1,2-α]pyrazin-8-yl)amino)-2-ethylbenzamide

To a solution of tert-butyl N-[2-[2-[[4-[[3-[3-cyano-1-(cyanomethyl)pyrazol-4-yl]imidazo[1,2-α]pyrazin-8-yl]amino]-2-ethyl-benzoyl]amino]ethoxy]ethyl]carbamate (60.0 mg, 0.100 mmol, 1 eq) in DCM (2 mL) was added trifluoroacetic acid (1.0 mL, 12.98 mmol, 129.51 eq), the reaction mixture was stirred at 20° C. for 16 h. The mixture was then concentrated and the obtained residue was purified by Prep-HPLC to afford N-[2-(2-aminoethoxy)ethyl]-4-[[3-[3-cyano-1-(cyanomethyl)pyrazol-4-yl]imidazo[1,2-α]pyrazin-8-yl]amino]-2-ethyl-benzamide; formic acid (18.9 mg, 0.030 mmol, 36.09% yield) as light yellow solid. MS(m/e): 499.1 (M+H).

### Example 831

### N-(2-(2-aminoethoxy)ethyl)-4-((3-(1-(cyanomethyl)-3-(methylthio)-1H-pyrazol-4-yl)imidazo[1,2-α]pyrazin-8-yl)amino)-2-ethylbenzamide

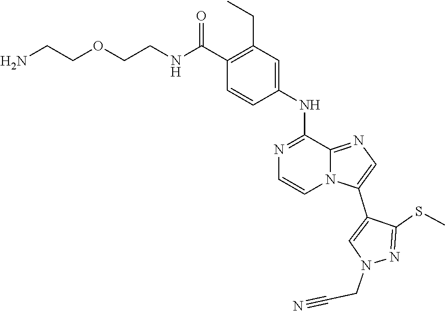 [SMILES: CCc1cc(Nc2nccn3c(-c4cn(CC#N)nc4SC)cnc23)ccc1C(=O)NCCOCCN]

Step 1:

### 4-iodo-3-(methylthio)-1H-pyrazole

A solution of N,N-dimethyl-5-methylsulfanyl-pyrazole-1-sulfonamide (26.8 g, 121.1 mmol, 1 eq) in Water (100 mL) and sulfuric acid (100.0 mL, 121.1 mmol, 1 eq) was stirred at 0° C. for 0.5 h, then N-iodosuccinimide (29.97 g, 133.21 mmol, 1.1 eq) was added to the solution, the mixture was stirred at 0° C. for 16 h. The mixture was slowly poured into sat.Na₂CO₃ solution and extracted with EtOAc, the combined organic phase was washed with sat.Na₂SO₃ solution and brine, dried over Na₂SO₄ and concentrated to afford crude product 4-iodo-3-methylsulfanyl-1H-pyrazole (28.5 g, 118.72 mmol, 88.23% yield) as yellow oil, which used for next step directly. MS(m/e): 241.5 (M+H).

Step 2:

### 4-iodo-3-(methylthio)-1-trityl-1H-pyrazole

To a solution of 4-iodo-3-methylsulfanyl-1H-pyrazole (23.0 g, 95.81 mmol, 1 eq) in THF (230 mL) was added sodium hydride, 60% in oil (4.6 g, 114.97 mmol, 1.2 eq) at 0° C., the mixture was stirred for 0.5 h at 0° C., then triphenylmethyl chloride (29.38 g, 105.39 mmol, 1.1 eq) was added to the solution in positions, the mixture was then warmed to 10° C. and stirred for another 16 h. The mixture was quenched with sat.NH₄Cl and extracted with EtOAc, the combined organic phase was washed with brine, dried over sodium sulfate, filtered and concentrated in vacuum to give crude product, which was purified by trituration with MTBE to afford 4-iodo-3-methylsulfanyl-1-trityl-pyrazole (25 g, 51.83 mmol, 54.1% yield) as white solid. MS(m/e): 505.0 (M+H).

Step 3:

### (3-(methylthio)-1-trityl-1H-pyrazol-4-yl)boronic acid

To a solution of 4-iodo-3-methylsulfanyl-1-trityl-pyrazole (20.0 g, 41.46 mmol, 1 eq) and boron isopropoxide (14.35 mL, 62.19 mmol, 1.5 eq) in THF (200 mL) was added drop wise butyllithium solution (29.85 mL, 74.63 mmol, 1.8 eq) at −78° C. under N₂ and the mixture was stirred for 3 h at 0° C. The mixture was poured into NH₄Cl solution and extracted with EtOAc, the combined organic phase was concentrated to afford crude product (3-methylsulfanyl-1-trityl-pyrazol-4-yl)boronic acid (21 g, 52.46 mmol, 88.57% yield) as colorless oil, which used for next step without purification. MS(m/e): 423.0 (M+H).

Step 4:

### methyl 2-ethyl-4-((3-(3-(methylthio)-1-trityl-1H-pyrazol-4-yl)imidazo[1,2-α]pyrazin-8-yl)amino)benzoate

A mixture of methyl 2-ethyl-4-[(3-iodoimidazo[1,2-α]pyrazin-8-yl)amino]benzoate (4.0 g, 9.47 mmol, 1 eq), (3-methylsulfanyl-1-trityl-pyrazol-4-yl)boronic acid (7.04 g, 12.32 mmol, 1.3 eq), [1,1′-bis(diphenylphosphino)ferrocene]dichloropalladium(II) (693.21 mg, 0.950 mmol, 0.1 eq) and sodium carbonate (2.01 g, 18.95 mmol, 2 eq) in 1,4-dioxane (60 mL)/water (6 mL) was stirred under N2 at 85° C. for 16 h. The mixture was filtered, the filtrate was concentrated and purified by silica column to afford methyl 2-ethyl-4-[[3-(3-methylsulfanyl-1-trityl-pyrazol-4-yl)imidazo[1,2-α]pyrazin-8-yl]amino]benzoate (3.5 g, 5.38 mmol, 56.77% yield) as light yellow solid. MS(m/e): 651.2 (M+H).

Step 5:

### methyl 2-ethyl-4-((3-(3-(methylthio)-1H-pyrazol-4-yl)imidazo[1,2-α]pyrazin-8-yl)amino)benzoate

A solution of methyl 2-ethyl-4-[[3-(3-methylsulfanyl-1-trityl-pyrazol-4-yl)imidazo[1,2-α]pyrazin-8-yl]amino]benzoate (3.5 g, 5.38 mmol, 1 eq) in hydrochloric acid solution (40.0 mL, 160 mmol, 4N in MeOH) was stirred at 25° C. for 16 h. The mixture was concentrated and purified by trituration with MTBE to afford methyl 2-ethyl-4-[[3-(3-methylsulfanyl-1H-pyrazol-4-yl)imidazo[1,2-α]pyrazin-8-yl]amino]benzoate (2.1 g, 5.14 mmol, 95.59% yield) as light yellow solid. MS(m/e): 409.4 (M+H).

Step 6:

### 2-ethyl-4-((3-(3-(methylthio)-1H-pyrazol-4-yl)imidazo[1,2-α]pyrazin-8-yl)amino)benzoic acid

To a solution of methyl 2-ethyl-4-[[3-(3-methylsulfanyl-1H-pyrazol-4-yl)imidazo[1,2-α]pyrazin-8-yl]amino]benzoate (2.0 g, 4.9 mmol, 1 eq) in THF (20 mL), Water (20 mL) and Methanol (20 mL) was added hydroxylithium hydrate (1.03 g, 24.48 mmol, 5 eq), the mixture was stirred at 25° C. for 40 h. The mixture was concentrated to remove MeOH and THF, then the mixture was acidified to PH=6 with HCl solution (1N), a lot of white solid appeared. Then the mixture was filtered and the filter cake was dried under vacume to afford 2-ethyl-4-[[3-(3-methylsulfanyl-1H-pyrazol-4-yl)imidazo[1,2-α]pyrazin-8-yl]amino]benzoic acid (1.9 g, 4.82 mmol, 98.38% yield) as white solid. MS(m/e): 395.4 (M+H).

Step 7:

### tert-butyl (2-(2-(2-ethyl-4-((3-(3-(methylthio)-1H-pyrazol-4-yl)imidazo[1,2-α]pyrazin-8-yl)amino)benzamido)ethoxy)ethyl)carbamate

A solution of 2-ethyl-4-[[3-(3-methylsulfanyl-1H-pyrazol-4-yl)imidazo[1,2-α]pyrazin-8-yl]amino]benzoic acid (300.0 mg, 0.760 mmol, 1 eq), N—BOC-2-(2-amino-ethoxy)-ethylamine (233.04 mg, 1.14 mmol, 1.5 eq) and triethylamine (0.42 mL, 3.04 mmol, 4 eq) in THF (15 mL) was added PROPYLPHOSPHONIC ANHYDRIDE (0.68 mL, 1.14 mmol, 1.5 eq) at 10° C., then the solution was stirred at 25° C. for 2 h. The mixture was poured into water and extracted with EtOAc, the combined organic phase was washed with brine (50 ml), dried over sodium sulfate and concentrated to afford a residue, which was purified by Prep-HPLC to afford tert-butyl N-[2-[2-[[2-ethyl-4-[[3-(3-methylsulfanyl-1H-pyrazol-4-yl)imidazo[1,2-α]pyrazin-8-yl]amino]benzoyl]amino]ethoxy]ethyl]carbamate (260 mg, 0.450 mmol, 58.87% yield) as light yellow oil. MS(m/e): 581.1 (M+H).

Step 8:

### tert-butyl (2-(2-(4-((3-(1-(cyanomethyl)-3-(methylthio)-1H-pyrazol-4-yl)imidazo[1,2-α]pyrazin-8-yl)amino)-2-ethylbenzamido)ethoxy)ethyl)carbamate

To mixture of tert-butyl N-[2-[2-[[2-ethyl-4-[[3-(3-methylsulfanyl-1H-pyrazol-4-yl)imidazo[1,2-α]pyrazin-8-yl]amino]benzoyl]amino]ethoxy]ethyl]carbamate (130.0 mg, 0.220 mmol, 1 eq) and potassium carbonate (92.82 mg, 0.670 mmol, 3 eq) in ACN (6.25 mL) was added bromoacetonitrile (80.56 mg, 0.670 mmol, 3 eq) at 0° C., then the mixture was warmed to 25° C. and stirred for 16 h. The mixture was filtered, the filtrate was concentrated and purified by Prep-HPLC to afford tert-butyl N-[2-[2-[[4-[[3-[1-(cyanomethyl)-3-methylsulfanyl-pyrazol-4-yl]imidazo[1,2-α]pyrazin-8-yl]amino]-2-ethyl-benzoyl]amino]ethoxy]ethyl]carbamate (86 mg, 0.140 mmol, 61.99% yield) as light yellow oil. MS(m/e): 620.3 (M+H).

Step 9:

### N-(2-(2-aminoethoxy)ethyl)-4-((3-(1-(cyanomethyl)-3-(methylthio)-1H-pyrazol-4-yl)imidazo[1,2-α]pyrazin-8-yl)amino)-2-ethylbenzamide

To solution of tert-butyl N-[2-[2-[[4-[[3-[1-(cyanomethyl)-3-methylsulfanyl-pyrazol-4-yl]imidazo[1,2-α]pyrazin-8-yl]amino]-2-ethyl-benzoyl]amino]ethoxy]ethyl]carbamate (86.0 mg, 0.140 mmol, 1 eq) in DCM (2 mL) was added trifluoroacetic acid (1 mL, 11.16 mmol, 80 eq) at 25° C., the mixture was stirred at 25° C. for 2 h. The mixture was concentrated and purified by Prep-HPLC to afford N-[2-(2-aminoethoxy)ethyl]-4-[[3-[1-(cyanomethyl)-3-methylsulfanyl-pyrazol-4-yl]imidazo[1,2-α]pyrazin-8-yl]amino]-2-ethyl-benzamide; formic acid (10.1 mg, 0.020 mmol, 13.84% yield) as white solid. MS(m/e): 520.3 (M+H).

### Example 832

### N-(2-(2-aminoethoxy)ethyl)-4-((3-(3-cyano-1-(2,2-difluoroethyl)-1H-pyrazol-4-yl)imidazo[1,2-α]pyrazin-8-yl)amino)-2-ethylbenzamide

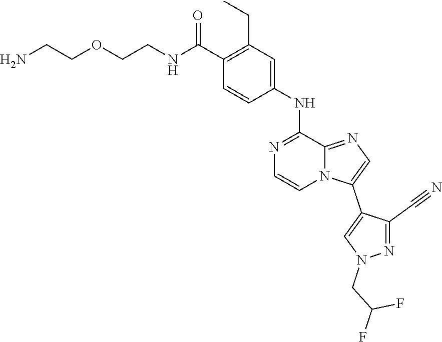 [SMILES: CCc1cc(Nc2nccn3c(-c4cn(CC(F)F)nc4C#N)cnc23)ccc1C(=O)NCCOCCN]

Step 1:

### methyl 4-(8-chloroimidazo[1,2-α]pyrazin-3-yl)-1H-pyrazole-3-carboxylate

A mixture of methyl 4-(8-chloroimidazo[1,2-α]pyrazin-3-yl)-1-trityl-pyrazole-3-carboxylate (5.5 g, 10.58 mmol, 1 eq) in hydrochloride solution (60.0 mL, 240 mmol, 4N in dioxane) was stirred at 30° C. for 16 h. The reaction mixture was concentrated to afford a residue which was triturated with MTBE. The mixture was filtered and the filter cake was concentrated to give methyl 4-(8-chloroimidazo[1,2-α]pyrazin-3-yl)-1H-pyrazole-3-carboxylate (2.5 g, 9 mmol, 85.12% yield) as orange solid. MS(m/e): 278.5 (M+H).

Step 2:

### methyl 4-(8-chloroimidazo[1,2-α]pyrazin-3-yl)-1-(2,2-difluoroethyl)-1H-pyrazole-3-carboxylate

To a mixture of methyl 4-(8-chloroimidazo[1,2-α]pyrazin-3-yl)-1H-pyrazole-3-carboxylate (2.0 g, 7.2 mmol, 1 eq) and potassium carbonate (2.99 g, 21.61 mmol, 3 eq) in ACN (40 mL) was added 2,2-difluoroethyl trifluoromethanesulfonate (1.29 mL, 10.8 mmol, 1.5 eq) at 10° C., then the mixture was stirred at 10° C. for 16 h. The mixture was filtrated and the filtrate was concentrated to afford the residue, which was purified by column to afford methyl 4-(8-chloroimidazo[1,2-α]pyrazin-3-yl)-1-(2,2-difluoroethyl)pyrazole-3-carboxylate (600 mg, 1.76 mmol, 24.38% yield) as red solid. MS(m/e): 342.2 (M+H).

Step 3:

### 4-(8-chloroimidazo[1,2-α]pyrazin-3-yl)-1-(2,2-difluoroethyl)-1H-pyrazole-3-carboxylic acid

To a solution of methyl 4-(8-chloroimidazo[1,2-α]pyrazin-3-yl)-1-(2,2-difluoroethyl)pyrazole-3-carboxylate (50.0 mg, 0.150 mmol, 1 eq) in THF (1 mL) and water (1 mL) was added hydroxylithium hydrate (30.7 mg, 0.730 mmol, 5 eq) at 30° C., the mixture was stirred at 30° C. for 2 h. The reaction mixture was diluted with H₂O and adjusted to pH=6 by HCl solution (1N). The reaction mixture was then extracted with AcOEt. The combined organic layer was dry over Na₂SO₄, filtered and concentrated in vacuum to give 4-(8-chloroimidazo[1,2-α]pyrazin-3-yl)-1-(2,2-difluoroethyl)pyrazole-3-carboxylic acid (40 mg, 0.120 mmol, 83.43% yield) as yellow solid. MS(m/e): 328.0 (M+H).

Step 4:

### 4-(8-chloroimidazo[1,2-α]pyrazin-3-yl)-1-(2,2-difluoroethyl)-1H-pyrazole-3-carboxamide

To a mixture of 4-(8-chloroimidazo[1,2-α]pyrazin-3-yl)-1-(2,2-difluoroethyl)pyrazole-3-carboxylic acid (300.0 mg, 0.920 mmol, 1 eq), ammonium chloride (1.29 mL, 1.83 mmol, 2 eq) and N,N-diisopropylethylamine (0.48 mL, 2.75 mmol, 3 eq) in DMF was added O-(7-azabenzotriazol-1-yl)-N,N,N′,N′-tetramethyluronium hexafluorophosphate (382.93 mg, 1.01 mmol, 1.1 eq), then the reaction mixture was stirred at 20° C. for 16 h. The reaction mixture was filtered and concentrated, the obtained crude product was purified by prep-HPLC to afford 4-(8-chloroimidazo[1,2-α]pyrazin-3-yl)-1-(2,2-difluoroethyl)pyrazole-3-carboxamide (200 mg, 0.610 mmol, 66.87% yield) as yellow solid. MS(m/e): 326.9 (M+H).

Step 5:

### 4-(8-chloroimidazo[1,2-α]pyrazin-3-yl)-1-(2,2-difluoroethyl)-1H-pyrazole-3-carbonitrile

To a stirred solution of 4-(8-chloroimidazo[1,2-α]pyrazin-3-yl)-1-(2,2-difluoroethyl)pyrazole-3-carboxamide (200.0 mg, 0.610 mmol, 1 eq) in DCM (5 mL) was added Burgess reagent (218.83 mg, 0.920 mmol, 1.5 eq) at 30° C., then the solution was stirred at 30° C. for 16 h. The reaction mixture was diluted with H₂O (and extracted with DCM. The combined organic layer was concentrated in vacuum to give the crude product. The crude product was purified by column to afford 4-(8-chloroimidazo[1,2-α]pyrazin-3-yl)-1-(2,2-difluoroethyl)pyrazole-3-carbonitrile (100 mg, 0.320 mmol, 52.92% yield) as yellow solid. MS(m/e): 309.0 (M+H).

Step 6:

### tert-butyl (2-(2-(4-((3-(3-cyano-1-(2,2-difluoroethyl)-1H-pyrazol-4-yl)imidazo[1,2-α]pyrazin-8-yl)amino)-2-ethylbenzamido)ethoxy)ethyl)carbamate

A solution of 4-(8-chloroimidazo[1,2-α]pyrazin-3-yl)-1-(2,2-difluoroethyl)pyrazole-3-carbonitrile (100.0 mg, 0.320 mmol, 1 eq) and tert-butyl N-[2-[2-[(4-amino-2-ethyl-benzoyl)amino]ethoxy]ethyl]carbamate (113.86 mg, 0.320 mmol, 1 eq) in MCCN (5 mL) and AcOH (0.5 mL) was stirred at 80° C. for 16 h. The solution was concentrated and the obtained residue was purified by prep-HPLC (FA) to afford tert-butyl N-[2-[2-[[4-[[3-[3-cyano-1-(2,2-difluoroethyl)pyrazol-4-yl]imidazo[1,2-α]pyrazin-8-yl]amino]-2-ethyl-benzoyl]amino]ethoxy]ethyl]carbamate (100 mg, 0.160 mmol, 49.49% yield) as white solid. MS(m/e): 624.3 (M+H).

Step 7:

### N-(2-(2-aminoethoxy)ethyl)-4-((3-(3-cyano-1-(2,2-difluoroethyl)-1H-pyrazol-4-yl)imidazo[1,2-α]pyrazin-8-yl)amino)-2-ethylbenzamide

To a stirred solution of tert-butyl N-[2-[2-[[4-[[3-[3-cyano-1-(2,2-difluoroethyl)pyrazol-4-yl]imidazo[1,2-α]pyrazin-8-yl]amino]-2-ethyl-benzoyl]amino]ethoxy]ethyl]carbamate (150.0 mg, 0.240 mmol, 1 eq) in DCM (7.5 mL) was added trifluoroacetic acid (0.19 mL, 2.41 mmol, 10 eq) at 30° C., then the reaction was stirred 30° C. for 2 h. The reaction mixture was concentrated and the obtained residue was purified by prep-HPLC to afford N-[2-(2-aminoethoxy)ethyl]-4-[[3-[3-cyano-1-(2,2-difluoroethyl)pyrazol-4-yl]imidazo[1,2-α]pyrazin-8-yl]amino]-2-ethyl-benzamide (66.6 mg, 0.130 mmol, 52.36% yield) as white solid. MS(m/e): 524.2 (M+H).

### Example 833

### N-(2-(2-aminoethoxy)ethyl)-4-((3-(3-chloro-1-(cyanomethyl)-1H-pyrazol-4-yl)imidazo[1,2-α]pyrazin-8-yl)amino)-2-ethylbenzamide

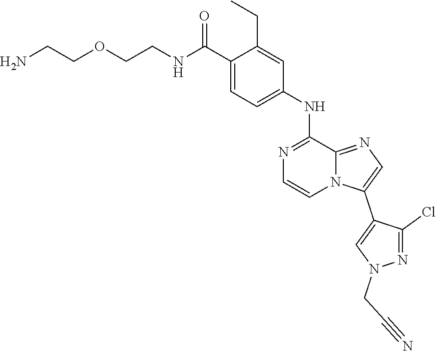 [SMILES: CCc1cc(Nc2nccn3c(-c4cn(CC#N)nc4Cl)cnc23)ccc1C(=O)NCCOCCN]

Step 1:

### 3-chloro-4-iodo-1-trityl-1H-pyrazole

To a solution of 3-chloro-4-iodo-1H-pyrazole (2.0 g, 8.76 mmol, 1 eq) in THF was added sodium hydride (60% in oil) (0.42 g, 10.51 mmol, 1.2 eq) at 0° C., the mixture was stirred at 0° C. for 0.5 h, then triphenylmethyl chloride (2.69 g, 9.63 mmol, 1.1 eq) was added to the solution. The mixture was stirred for 16 h at 30° C. Then, the mixture was quenched with sat.NH₄Cl (100 mL) and extracted with EtOAc, the combined organic phase was washed with brine, dried over sodium sulfate, filtered and concentrated in vacuum to give the crude product, which was purified by trituration with MTBE to afford 3-chloro-4-iodo-1-trityl-pyrazole (4 g, 8.5 mmol, 97.05% yield) as white solid. MS(m/e): 492.9 (M+Na).

Step 2:

### (3-chloro-1-trityl-1H-pyrazol-4-yl)boronic acid

To a solution of 3-chloro-4-iodo-1-trityl-pyrazole (4.0 g, 8.5 mmol, 1 eq) and boron isopropoxide (2.94 mL, 12.75 mmol, 1.5 eq) in THF (40 mL) was added drop wise butyllithium solution (6.12 mL, 15.3 mmol, 1.8 eq) at −70° C. under N₂, then the mixture was stirred at −70° C. for 2 h. The mixture was poured into NH₄Cl solution and extracted with EtOAc, the combined organic phase was concentrated to afford crude (3-chloro-1-trityl-pyrazol-4-yl)boronic acid (3 g, 7.72 mmol, 90.84% yield) as light yellow solid, which used for next step without purification. MS(m/e): 411.1 (M+Na).

Step 3:

### 8-chloro-3-(3-chloro-1-trityl-1H-pyrazol-4-yl)imidazo[1,2-α]pyrazine

A mixture of 8-chloro-3-iodo-imidazo[1,2-α]pyrazine (2.0 g, 7.16 mmol, 1 eq), (3-chloro-1-trityl-pyrazol-4-yl)boronic acid (3.48 g, 7.16 mmol, 1 eq), sodium carbonate (1.52 g, 14.31 mmol, 2 eq), [1,1′-bis(diphenylphosphino)ferrocene]dichloropalladium(II) (0.26 g, 0.360 mmol, 0.050 eq) in 1,4-dioxane (25 mL) and water (2 mL) was stirred under N₂ at 80° C. for 16 h. The mixture was filtered and the filtrate was concentrated and purified by silica column to afford 8-chloro-3-(3-chloro-1-trityl-pyrazol-4-yl)imidazo[1,2-α]pyrazine (450 mg, 0.910 mmol, 12.67% yield) as white solid. MS(m/e): 496.0 (M+H).

Step 4:

### 8-chloro-3-(3-chloro-1H-pyrazol-4-yl)imidazo[1,2-α]pyrazine

A mixture of 8-chloro-3-(3-chloro-1-trityl-pyrazol-4-yl)imidazo[1,2-α]pyrazine (450.0 mg, 0.910 mmol, 1 eq), in hydrochloride solution (10.0 mL, 1.81 mmol, 4N in dioxane) was stirred at 30° C. for 16 h. The mixture was diluted with MTBE, white solid appeared, the mixture was filtered and the filter cake was dried under vacume to afford 8-chloro-3-(3-chloro-1H-pyrazol-4-yl)imidazo[1,2-α]pyrazine (220 mg, 0.870 mmol, 95.51% yield) as yellow solid MS(m/e): 254.1 (M+H).

Step 5:

### 2-(3-chloro-4-(8-chloroimidazo[1,2-α]pyrazin-3-yl)-1H-pyrazol-1-yl)acetonitrile

To a mixture of 8-chloro-3-(3-chloro-1H-pyrazol-4-yl)imidazo[1,2-α]pyrazine (220.0 mg, 0.870 mmol, 1 eq) and potassium carbonate (239.34 mg, 1.73 mmol, 2 eq) in ACN (7 mL) was added bromoacetonitrile (0.09 mL, 1.3 mmol, 1.5 eq) at 0° C., then the mixture was warmed to 30° C. and stirred for 16 h. The mixture was filtered, the filtrate was concentrated and purified by reversed-phase HPLC to afford 2-[3-chloro-4-(8-chloroimidazo[1,2-α]pyrazin-3-yl)pyrazol-1-yl]acetonitrile (220 mg, 0.750 mmol, 86.68% yield) as yellow oil. MS(m/e): 292.8 (M+H).

Step 6:

### tert-butyl (2-(2-(4-((3-(3-chloro-1-(cyanomethyl)-1H-pyrazol-4-yl)imidazo[1,2-α]pyrazin-8-yl)amino)-2-ethylbenzamido)ethoxy)ethyl)carbamate

A mixture of 2-[3-chloro-4-(8-chloroimidazo[1,2-α]pyrazin-3-yl)pyrazol-1-yl]acetonitrile (200.0 mg, 0.680 mmol, 1 eq) and tert-butyl N-[2-[2-[(4-amino-2-ethyl-benzoyl)amino]ethoxy]ethyl]carbamate (0.09 mL, 0.680 mmol, 1 eq) in ACN (9 mL) and acetic acid (1 mL) was stirred at 60° C. for 16 h. The mixture was concentrated and purified by reversed-phase HPLC to afford tert-butyl N-[2-[2-[[4-[[3-[3-chloro-1-(cyanomethyl)pyrazol-4-yl]imidazo[1,2-α]pyrazin-8-yl]amino]benzoyl]amino]ethoxy]ethyl]carbamate; propane (150 mg, 0.250 mmol, 36.22% yield) as light yellow oil. MS(m/e): 608.2 (M+H).

Step 7:

### N-(2-(2-aminoethoxy)ethyl)-4-((3-(3-chloro-1-(cyanomethyl)-1H-pyrazol-4-yl)imidazo[1,2-α]pyrazin-8-yl)amino)-2-ethylbenzamide

To solution of tert-butyl N-[2-[2-[[4-[[3-[3-chloro-1-(cyanomethyl)pyrazol-4-yl]imidazo[1,2-α]pyrazin-8-yl]amino]-2-ethyl-benzoyl]amino]ethoxy]ethyl]carbamate (150.49 mg, 0.250 mmol, 1 eq) in DCM (5 mL) was added trifluoroacetic acid (5.0 mL, 64.9 mmol, 262.25 eq) at 30° C., the mixture was stirred at 30° C. for 1 h. The mixture was concentrated and purified by Prep-HPLC to afford N-[2-(2-aminoethoxy)ethyl]-4-[[3-[3-chloro-1-(cyanomethyl)pyrazol-4-yl]imidazo[1,2-α]pyrazin-8-yl]amino]-2-ethyl-benzamide (17.4 mg, 0.030 mmol, 13.58% yield) as white solid. MS(m/e): 508.3 (M+H).

Assay Procedures

Antimicrobial Susceptibility Testing:

90% Growth Inhibitory Concentration (IC90) Determination

The in vitro antimicrobial activity of the compounds was determined according to the following procedure:

The assay used a 10-points Iso-Sensitest broth medium to measure quantitatively the in vitro activity of the compounds against Acinetobacter baumannii ATCC17961 or ATCC17968.

Stock compounds in DMSO were serially twofold diluted (e.g. range from 50 to 0.097 μM final concentration) in 384 wells microtiter plates and inoculated with 49 μl the bacterial suspension in Iso-Sensitest medium to have a final cell concentration of ˜5×10⁽⁵⁾ CFU/ml in a final volume/well of 50 ul/well. Microtiter plates were incubated at 35±2° C.

Bacterial cell growth was determined with the measurement of optical density at λ=600 nm each 20 minutes over a time course of 16 h. Growth inhibition was calculated during the logarithmic growth of the bacterial cells with determination of the concentration inhibiting 50% (IC50) and 90% (IC90) of the growth.

Table 7 provides the 90% growth inhibitory concentrations (IC90) in micromoles per liter of the compounds of present invention obtained against the strain Acinetobacter baumannii ATCC17961 and/or ATCC17968.

Particular compounds of the present invention exhibit an IC90 (Acinetobacter baumannii ATCC17961 and/or ATCC17968)≤25 μmol/A.

More particular compounds of the present invention exhibit an IC90 (Acinetobacter baumannii ATCC17961 and/or ATCC17968)≤5 μmol/A.

Most particular compounds of the present invention exhibit an IC90 (Acinetobacter baumannii ATCC17961 and/or ATCC17968)≤1 μmol/A.

TABLE 7
Activity data

| | A. baumannii | A. baumannii |
|---|---|---|
| | ATCC 17978 | ATCC 17961 |
| Ex | IC90 (uM) | IC90 (uM) |
| 1 | 0.93 | 0.623 |
| 2 | | 0.513 |
| 3 | | 0.16 |
| 4 | | 0.132 |
| 5 | | 0.262 |
| 6 | | 0.159 |
| 7 | | 0.782 |
| 8 | | 0.484 |
| 9 | | 1.052 |
| 10 | | 0.458 |
| 11 | | 0.204 |
| 12 | | 0.176 |
| 13 | | 0.166 |
| 14 | | 0.141 |
| 15 | | 0.251 |
| 16 | | 0.069 |
| 17 | | 0.335 |
| 18 | | 0.26 |
| 20 | | 0.462 |
| 21 | 4.43 | |
| 22 | 1.84 | 1.841 |
| 23 | 0.297 | 0.128 |
| 24 | | 0.523 |
| 25 | | 0.141 |
| 26 | | 0.282 |
| 27 | | 0.306 |
| 28 | | 0.369 |
| 29 | | 0.107 |
| 30 | | 0.159 |
| 31 | | 0.175 |
| 32 | | 0.19 |
| 33 | | 0.269 |
| 34 | | 0.367 |
| 35 | | 0.185 |
| 36 | | 0.348 |
| 37 | | 0.239 |
| 38 | | 0.327 |
| 39 | | 0.47 |
| 40 | | 0.196 |
| 41 | | 0.254 |
| 42 | | 0.354 |
| 43 | | 1.949 |
| 44 | | 0.059 |
| 45 | | 0.074 |
| 46 | | 0.093 |
| 47 | | 0.105 |
| 48 | | 0.117 |
| 49 | | 0.12 |
| 50 | | 0.121 |
| 51 | | 0.13 |
| 52 | | 0.144 |
| 53 | | 0.157 |
| 54 | | 0.162 |
| 55 | | 0.167 |
| 56 | | 0.194 |
| 57 | | 0.202 |
| 58 | | 0.209 |
| 59 | | 0.216 |
| 60 | | 0.23 |
| 61 | | 0.232 |
| 62 | | 0.242 |
| 63 | | 0.27 |
| 64 | | 0.306 |
| 65 | | 0.339 |
| 66 | | 0.379 |
| 67 | | 0.413 |
| 68 | | 0.43 |
| 69 | | 0.433 |
| 70 | | 0.489 |
| 71 | | 0.593 |
| 72 | | 0.599 |
| 73 | | 0.687 |
| 74 | | 0.781 |
| 75 | | 1.701 |
| 76 | | 2.086 |
| 77 | | 3.29 |
| 78 | | 0.161 |
| 79 | | 0.222 |
| 80 | | 0.737 |
| 81 | | 1.065 |
| 82 | | 1.153 |
| 83 | | 2.465 |
| 84 | | 2.977 |
| 85 | | 3.263 |
| 86 | | 3.381 |
| 87 | | 0.359 |
| 88 | | 0.431 |
| 89 | | 0.573 |
| 90 | | 0.643 |
| 91 | | 0.645 |
| 92 | | 0.947 |
| 93 | | 1.024 |
| 94 | | 1.239 |
| 95 | | 1.531 |
| 96 | | 1.634 |
| 97 | | 1.7 |
| 98 | | 1.941 |
| 99 | | 2.079 |
| 100 | | 0.1 |
| 101 | | 0.138 |
| 102 | | 0.18 |
| 103 | | 0.195 |
| 104 | | 0.227 |
| 105 | | 1.561 |
| 106 | | 0.222 |
| 107 | | 0.239 |
| 108 | | 0.302 |
| 109 | | 0.333 |
| 110 | | 0.506 |
| 111 | | 0.997 |
| 112 | | 0.231 |
| 113 | | 0.234 |
| 114 | | 0.329 |
| 115 | | 0.251 |
| 116 | | 0.22 |
| 117 | | 0.338 |
| 118 | | 0.343 |
| 119 | | 0.476 |
| 120 | | 0.109 |
| 121 | | 0.201 |
| 122 | | 0.208 |
| 123 | | 0.209 |
| 124 | | 0.121 |
| 125 | | 2.233 |
| 126 | | 4.146 |
| 127 | | >5.000 |
| 128 | | 0.659 |
| 129 | | 1.064 |
| 130 | | 1.326 |
| 131 | | 0.117 |
| 132 | | 0.136 |
| 133 | | 0.322 |
| 134 | | 0.768 |
| 135 | | 0.933 |
| 136 | | 1.335 |
| 137 | | 1.864 |
| 138 | | 2.008 |
| 139 | | 2.798 |
| 140 | | 3.314 |
| 141 | | 4.05 |
| 142 | | 0.554 |
| 143 | | 0.555 |
| 144 | | 0.335 |
| 145 | | 1.903 |
| 146 | | 0.028 |
| 147 | | 0.049 |
| 148 | | 0.084 |
| 149 | | 0.102 |
| 150 | | 0.173 |
| 151 | | 0.204 |
| 152 | | 0.241 |
| 153 | | 0.311 |
| 154 | | 0.391 |
| 155 | | 0.585 |
| 156 | | 0.627 |
| 157 | | 0.969 |
| 158 | | 4.028 |
| 159 | | 0.408 |
| 160 | | 0.63 |
| 161 | | 0.764 |
| 162 | | 1.217 |
| 163 | | 1.271 |
| 164 | | 1.93 |
| 165 | | 2.515 |
| 166 | | 4.48 |
| 167 | | 0.485 |
| 168 | | 0.369 |
| 169 | | 0.392 |
| 170 | | 0.475 |
| 171 | | 0.509 |
| 172 | | 0.832 |
| 173 | | 1.107 |
| 174 | | 0.081 |
| 175 | | 0.357 |
| 176 | | 0.409 |
| 177 | | 0.11 |
| 178 | | 0.161 |
| 179 | | 0.181 |
| 180 | | 0.182 |
| 181 | | 0.293 |
| 182 | | 0.368 |
| 183 | | 0.372 |
| 184 | | 0.4 |
| 185 | | 0.65 |
| 186 | | 0.206 |
| 187 | | 0.159 |
| 188 | | 0.197 |
| 189 | | 0.204 |
| 190 | | 0.245 |
| 191 | | 0.276 |
| 192 | | 0.208 |
| 193 | | 0.244 |
| 194 | | 0.274 |
| 195 | | 0.331 |
| 196 | | 0.674 |
| 197 | | 0.16 |
| 198 | | 0.216 |
| 199 | | 0.336 |
| 200 | | 0.372 |
| 201 | | 0.486 |
| 202 | | 0.109 |
| 203 | | 0.102 |
| 204 | | 0.256 |
| 205 | | 0.256 |
| 206 | | 0.169 |
| 207 | | 0.205 |
| 208 | | 0.232 |
| 209 | | 0.105 |
| 210 | | 0.174 |
| 211 | | 0.284 |
| 212 | | 0.296 |
| 213 | | 0.378 |
| 214 | | 0.349 |
| 215 | | 0.394 |
| 216 | | 0.446 |
| 217 | | 0.543 |
| 218 | | 0.574 |
| 219 | | 0.599 |
| 220 | | 0.51 |
| 221 | | 0.922 |
| 222 | | 1.093 |
| 223 | | 1.123 |
| 224 | | 1.5 |
| 225 | | 1.807 |
| 226 | | 0.088 |
| 227 | | 0.17 |
| 228 | | 0.409 |
| 229 | | 1.105 |
| 230 | | 0.091 |
| 231 | | 0.102 |
| 232 | | 0.124 |
| 233 | | 0.169 |
| 234 | | 0.189 |
| 235 | | 0.19 |
| 236 | | 0.193 |
| 237 | | 0.196 |
| 238 | | 0.2 |
| 239 | | 0.211 |
| 240 | | 0.253 |
| 241 | | 0.322 |
| 242 | | 0.33 |
| 243 | | 0.384 |
| 244 | | 0.411 |
| 245 | | 0.45 |
| 246 | | 0.533 |
| 247 | | 0.116 |
| 248 | | 0.311 |
| 249 | | 0.047 |
| 250 | | 0.084 |
| 251 | | 0.136 |
| 252 | | 0.169 |
| 253 | | 0.198 |
| 254 | | 0.222 |
| 255 | | 0.291 |
| 256 | | 0.358 |
| 257 | | 0.37 |
| 258 | | 0.398 |
| 259 | | 0.463 |
| 260 | | 0.519 |
| 261 | | 0.521 |
| 262 | | 1.1 |
| 263 | | 2.43 |
| 264 | | 0.451 |
| 265 | | 0.231 |
| 266 | | 0.282 |
| 267 | | 0.225 |
| 268 | | 0.346 |
| 269 | | 1.393 |
| 270 | | 2.656 |
| 271 | | 3.066 |
| 272 | | 0.085 |
| 273 | | 0.087 |
| 274 | | 0.101 |
| 275 | | 0.169 |
| 276 | | 0.178 |
| 277 | | 0.186 |
| 278 | | 0.332 |
| 279 | | 0.664 |
| 280 | | 0.517 |
| 281 | | 0.345 |
| 282 | | 0.701 |
| 283 | | 1.558 |
| 284 | | 0.177 |
| 285 | | 0.185 |
| 286 | | 0.226 |
| 287 | | 0.23 |
| 288 | | 0.233 |
| 289 | | 0.234 |
| 290 | | 0.33 |
| 291 | | 0.332 |
| 292 | | 0.354 |
| 293 | | 0.429 |
| 294 | | 0.457 |
| 295 | | 0.473 |
| 296 | | 0.491 |
| 297 | | 0.31 |
| 298 | | 1.321 |
| 299 | | 0.699 |
| 300 | | 0.905 |
| 301 | | 0.164 |
| 302 | | 0.17 |
| 303 | | 0.201 |
| 304 | | 0.253 |
| 305 | | 0.406 |
| 306 | | 0.483 |
| 307 | | 0.514 |
| 308 | | 0.525 |
| 309 | | 0.618 |
| 310 | | 0.719 |
| 311 | | 0.769 |
| 312 | | 0.839 |
| 313 | | 0.995 |
| 314 | | 1.425 |
| 315 | | 1.459 |
| 316 | | 1.521 |
| 317 | | 1.878 |
| 318 | | 0.411 |
| 319 | | 0.438 |
| 320 | | 0.705 |
| 321 | | 0.901 |
| 322 | | 0.97 |
| 323 | | 1.052 |
| 324 | | 1.228 |
| 325 | | 2.023 |
| 326 | | 0.135 |
| 327 | | 0.146 |
| 328 | | 0.157 |
| 329 | | 0.211 |
| 330 | | 0.275 |
| 331 | | 0.401 |
| 332 | | 0.509 |
| 333 | | 0.695 |
| 334 | | 0.782 |
| 335 | | 0.148 |
| 336 | | 0.152 |
| 337 | | 0.359 |
| 338 | | 0.691 |
| 339 | | 2.488 |
| 340 | | 0.194 |
| 341 | | 0.223 |
| 342 | | 0.228 |
| 343 | | 0.546 |
| 344 | | 0.572 |
| 345 | | 1.22 |
| 346 | | 0.164 |
| 347 | | 1.67 |
| 348 | | 1.548 |
| 349 | | 1.631 |
| 350 | | 0.371 |
| 351 | | 0.099 |
| 352 | | 0.229 |
| 353 | | 0.241 |
| 354 | | 0.918 |
| 355 | | 1.298 |
| 356 | | 0.068 |
| 357 | | 1.593 |
| 358 | | 0.189 |
| 359 | | 0.303 |
| 360 | | 0.257 |
| 361 | | 0.267 |
| 362 | | 0.35 |
| 363 | | 0.738 |
| 364 | | 1.126 |
| 365 | | 1.401 |
| 366 | | 0.296 |
| 367 | | 0.468 |
| 370 | | 0.177 |
| 371 | | 0.309 |
| 372 | | 0.957 |
| 373 | | 1.113 |
| 374 | | 0.237 |
| 375 | | 0.338 |
| 376 | | 0.43 |
| 378 | | 0.562 |
| 379 | | 0.576 |
| 380 | | 0.623 |
| 381 | | 0.63 |
| 382 | | 0.749 |
| 384 | | 0.833 |
| 385 | | 0.969 |
| 387 | | 4.838 |
| 388 | | 0.337 |
| 389 | | 0.136 |
| 390 | | 0.177 |
| 391 | | 0.203 |
| 392 | | 0.208 |
| 393 | | 0.239 |
| 394 | | 0.331 |
| 399 | | 0.214 |
| 400 | | 0.217 |
| 401 | | 0.273 |
| 402 | | 0.384 |
| 403 | | 0.398 |
| 404 | | 1.226 |
| 405 | | 1.296 |
| 406 | | 1.398 |
| 407 | | 2.965 |
| 408 | | 0.481 |
| 410 | | 1.418 |
| 413 | | 2.197 |
| 414 | | 4.04 |
| 415 | | 0.392 |
| 416 | | 0.441 |
| 417 | | 0.506 |
| 418 | | 0.583 |
| 419 | | 0.654 |
| 420 | | 0.746 |
| 421 | | 0.629 |
| 422 | | 0.63 |
| 423 | | 0.638 |
| 424 | | 0.685 |
| 425 | | 0.691 |
| 426 | | 0.948 |
| 428 | | 0.465 |
| 429 | | 1.068 |
| 430 | | 2.136 |
| 431 | | 0.449 |
| 432 | | 0.751 |
| 433 | | 2.664 |
| 434 | | 0.463 |
| 435 | | 2.686 |
| 436 | | 0.536 |
| 437 | | 1.02 |
| 439 | | 0.834 |
| 440 | | 0.497 |
| 441 | | 0.617 |
| 442 | | 0.622 |
| 443 | | 0.81 |
| 444 | | 1.426 |
| 445 | | 1.48 |
| 446 | | 2.273 |
| 447 | | 4.185 |
| 448 | | 0.235 |
| 449 | | 0.301 |
| 450 | | 0.306 |
| 451 | | 0.365 |
| 452 | | 0.401 |
| 453 | | 0.586 |
| 454 | | 0.778 |
| 455 | | 1.207 |
| 456 | | 2.737 |
| 457 | | 0.442 |
| 458 | | 0.551 |
| 459 | | 0.762 |
| 460 | | 0.896 |
| 461 | | 0.926 |
| 462 | | 0.964 |
| 463 | | 0.998 |
| 464 | | 1.264 |
| 465 | | 1.619 |
| 466 | | 2.944 |
| 467 | | 1.42 |
| 468 | | 1.426 |
| 469 | | 0.705 |
| 470 | | 0.793 |
| 471 | | 1.1 |
| 472 | | 1.237 |
| 473 | | 1.291 |
| 474 | | 1.337 |
| 475 | | 1.642 |
| 476 | | 2.011 |
| 477 | | 3.243 |
| 478 | | 3.458 |
| 479 | | 0.624 |
| 480 | | 1.84 |
| 481 | | 0.117 |
| 482 | | 0.174 |
| 483 | | 0.194 |
| 484 | | 0.154 |
| 485 | | 0.189 |
| 486 | | 0.208 |
| 487 | | 0.171 |
| 488 | | 0.147 |
| 489 | | 0.184 |
| 490 | | 0.196 |
| 491 | | 0.195 |
| 492 | | 0.2 |
| 493 | | 0.129 |
| 494 | | 0.157 |
| 495 | | 0.173 |
| 496 | | 0.173 |
| 497 | | 0.183 |
| 498 | | 0.193 |
| 499 | | 0.218 |
| 500 | | 0.222 |
| 501 | | 0.224 |
| 502 | | 0.233 |
| 503 | | 0.233 |
| 504 | | 0.238 |
| 505 | | 0.449 |
| 506 | | 0.191 |
| 507 | | 0.245 |
| 508 | | 0.198 |
| 509 | | 0.23 |
| 510 | | 0.347 |
| 511 | | 0.295 |
| 512 | | 0.341 |
| 513 | | 0.15 |
| 514 | | 0.803 |
| 515 | | 0.49 |
| 516 | | 0.14 |
| 517 | | 0.369 |
| 518 | | 0.935 |
| 519 | | 0.147 |
| 520 | | 0.27 |
| 521 | | 0.38 |
| 522 | | 0.402 |
| 523 | | 0.313 |
| 524 | | 0.395 |
| 525 | | 0.593 |
| 526 | | 0.306 |
| 527 | | 0.306 |
| 528 | | 0.243 |
| 529 | | 0.248 |
| 530 | | 0.327 |
| 531 | | 0.299 |
| 532 | | 0.245 |
| 534 | | 0.251 |
| 535 | | 0.247 |
| 536 | | 0.145 |
| 537 | | 0.163 |
| 538 | | 0.261 |
| 539 | | 0.461 |
| 540 | | 0.249 |
| 541 | | 0.246 |
| 542 | | 0.196 |
| 543 | | 0.162 |
| 544 | | 0.232 |
| 545 | | 0.306 |
| 546 | | 0.298 |
| 547 | | 0.298 |
| 548 | | 0.267 |
| 549 | | 0.235 |
| 550 | | 0.261 |
| 551 | | 0.357 |
| 552 | | 0.489 |
| 553 | | 0.682 |
| 554 | | 0.198 |
| 555 | | 0.977 |
| 556 | | 0.675 |
| 557 | | 0.144 |
| 558 | | 0.174 |
| 559 | | 0.398 |
| 560 | | 0.836 |
| 561 | | 0.909 |
| 562 | | 0.305 |
| 563 | | 0.575 |
| 564 | | 0.58 |
| 565 | | 0.188 |
| 566 | | 0.477 |
| 567 | | 0.476 |
| 568 | | 0.46 |
| 569 | | 0.364 |
| 570 | | 0.256 |
| 571 | | 0.192 |
| 572 | | 0.291 |
| 573 | | 0.169 |
| 574 | | 0.242 |
| 575 | | 0.175 |
| 576 | | 0.9 |
| 577 | | 0.395 |
| 578 | | 0.394 |
| 579 | | 0.538 |
| 580 | | 0.322 |
| 581 | | 0.354 |
| 582 | | 0.232 |
| 583 | | 0.347 |
| 584 | | 0.257 |
| 585 | | 0.325 |
| 586 | | 0.434 |
| 587 | | 0.221 |
| 588 | | 0.46 |
| 589 | | 0.84 |
| 590 | | 0.375 |
| 591 | | 0.483 |
| 592 | | 0.402 |
| 593 | | 0.234 |
| 594 | | 0.164 |
| 595 | | 0.186 |
| 596 | | 0.131 |
| 597 | | 0.179 |
| 598 | | 0.359 |
| 599 | | 0.522 |
| 600 | | 0.517 |
| 601 | | 0.422 |
| 602 | | 0.448 |
| 603 | | 0.384 |
| 604 | | 0.602 |
| 605 | | 0.496 |
| 606 | | 0.303 |
| 607 | | 0.878 |
| 608 | | 0.936 |
| 609 | | 0.086 |
| 610 | | 0.099 |
| 611 | | 0.11 |
| 612 | | 0.1 |
| 613 | | 0.105 |
| 614 | | 0.11 |
| 615 | | 0.122 |
| 616 | | 0.153 |
| 617 | | 0.125 |
| 618 | | 0.129 |
| 619 | | 0.131 |
| 620 | | 0.139 |
| 621 | | 0.144 |
| 622 | | 0.148 |
| 623 | | 0.161 |
| 624 | | 0.19 |
| 625 | | 0.176 |
| 626 | | 0.233 |
| 627 | | 0.19 |
| 628 | | 0.195 |
| 629 | | 0.198 |
| 630 | | 0.206 |
| 631 | | 0.209 |
| 632 | | 0.209 |
| 633 | | 0.214 |
| 634 | | 0.218 |
| 635 | | 0.22 |
| 636 | | 0.222 |
| 637 | | 0.226 |
| 638 | | 0.226 |
| 639 | | 0.228 |
| 640 | | 0.229 |
| 641 | | 0.231 |
| 642 | | 0.232 |
| 643 | | 0.232 |
| 644 | | 0.24 |
| 645 | | 0.242 |
| 646 | | 0.243 |
| 647 | | 0.244 |
| 648 | | 0.246 |
| 649 | | 0.247 |
| 650 | | 0.252 |
| 651 | | 0.254 |
| 652 | | 0.259 |
| 653 | | 0.259 |
| 654 | | 0.263 |
| 655 | | 0.266 |
| 656 | | 0.266 |
| 658 | | 0.269 |
| 659 | | 0.27 |
| 660 | | 0.277 |
| 661 | | 0.279 |
| 662 | | 0.284 |
| 663 | | 0.285 |
| 664 | | 0.286 |
| 665 | | 0.29 |
| 666 | | 0.291 |
| 667 | | 0.294 |
| 668 | | 0.294 |
| 669 | | 0.3 |
| 670 | | 0.304 |
| 671 | | 0.309 |
| 672 | | 0.313 |
| 673 | | 0.314 |
| 674 | | 0.315 |
| 675 | | 0.315 |
| 676 | | 0.316 |
| 677 | | 0.317 |
| 678 | | 0.321 |
| 679 | | 0.322 |
| 680 | | 0.326 |
| 681 | | 0.327 |
| 682 | | 0.329 |
| 683 | | 0.336 |
| 684 | | 0.34 |
| 685 | | 0.34 |
| 686 | | 0.343 |
| 687 | | 0.345 |
| 688 | | 0.349 |
| 689 | | 0.351 |
| 690 | | 0.351 |
| 691 | | 0.352 |
| 692 | | 0.356 |
| 693 | | 0.358 |
| 694 | | 0.358 |
| 695 | | 0.362 |
| 696 | | 0.37 |
| 697 | | 0.371 |
| 698 | | 0.374 |
| 699 | | 0.38 |
| 700 | | 0.382 |
| 701 | | 0.382 |
| 702 | | 0.383 |
| 703 | | 0.383 |
| 704 | | 0.386 |
| 705 | | 0.391 |
| 706 | | 0.394 |
| 707 | | 0.395 |
| 708 | | 0.397 |
| 709 | | 0.404 |
| 710 | | 0.408 |
| 711 | | 0.409 |
| 712 | | 0.41 |
| 713 | | 0.417 |
| 714 | | 0.422 |
| 715 | | 0.422 |
| 716 | | 0.426 |
| 717 | | 0.434 |
| 718 | | 0.435 |
| 719 | | 0.435 |
| 720 | | 0.441 |
| 721 | | 0.452 |
| 722 | | 0.458 |
| 723 | | 0.459 |
| 724 | | 0.459 |
| 725 | | 0.462 |
| 726 | | 0.463 |
| 727 | | 0.465 |
| 729 | | 0.473 |
| 730 | | 0.475 |
| 731 | | 0.476 |
| 732 | | 0.48 |
| 733 | | 0.481 |
| 734 | | 0.489 |
| 735 | | 0.49 |
| 736 | | 0.49 |
| 737 | | 0.495 |
| 738 | | 0.526 |
| 739 | | 0.527 |
| 740 | | 0.529 |
| 741 | | 0.53 |
| 742 | | 0.53 |
| 743 | | 0.532 |
| 744 | | 0.557 |
| 745 | | 0.569 |
| 746 | | 0.581 |
| 747 | | 0.598 |
| 748 | | 0.608 |
| 749 | | 0.619 |
| 750 | | 0.625 |
| 751 | | 0.627 |
| 752 | | 0.636 |
| 753 | | 0.659 |
| 754 | | 0.671 |
| 755 | | 0.678 |
| 756 | | 0.679 |
| 757 | | 0.689 |
| 758 | | 0.693 |
| 759 | | 0.693 |
| 760 | | 0.718 |
| 761 | | 0.721 |
| 762 | | 0.722 |
| 763 | | 0.75 |
| 764 | | 0.809 |
| 765 | | 0.895 |
| 766 | | 0.903 |
| 767 | | 0.904 |
| 768 | | 0.916 |
| 769 | | 0.928 |
| 770 | | 0.929 |
| 771 | | 0.953 |
| 772 | | 0.968 |
| 773 | | 0.978 |
| 774 | | 0.986 |
| 775 | | 0.988 |
| 776 | | 1.006 |
| 777 | | 1.018 |
| 778 | | 1.032 |
| 779 | | 1.033 |
| 780 | | 1.061 |
| 781 | | 1.093 |
| 782 | | 1.153 |
| 783 | | 1.209 |
| 784 | | 1.238 |
| 785 | | 1.32 |
| 786 | | 1.433 |
| 787 | | 1.566 |
| 788 | | 1.602 |
| 789 | | 1.608 |
| 790 | | 1.644 |
| 791 | | 1.85 |
| 792 | | 1.918 |
| 793 | | 1.93 |
| 794 | | 2.201 |
| 795 | | 2.233 |
| 796 | | 2.247 |
| 797 | | 2.36 |
| 798 | | 2.362 |
| 799 | | 2.743 |
| 800 | | 2.783 |
| 801 | | 3.001 |
| 802 | | 3.535 |
| 803 | | 3.772 |
| 804 | | 0.511 |
| 805 | | 0.88 |
| 806 | | 0.25 |
| 807 | | 0.31 |
| 808 | | 0.26 |
| 809 | | 0.17 |
| 810 | | 0.25 |
| 811 | | 0.23 |
| 812 | | 0.20 |
| 813 | | 0.21 |
| 814 | | 0.14 |
| 815 | | 0.18 |
| 816 | | 0.29 |
| 817 | | 0.18 |
| 818 | | 0.15 |
| 819 | | 0.18 |
| 820 | | 0.15 |
| 821 | | 0.16 |
| 822 | | 0.15 |
| 823 | | 0.18 |
| 824 | | 0.12 |
| 825 | | 0.11 |
| 826 | | 0.11 |
| 827 | | 0.57 |
| 828 | | 0.35 |
| 829 | | 0.72 |
| 830 | | 0.58 |
| 831 | | 0.32 |
| 832 | | 0.48 |
| 833 | | 0.46 |

### Example 834

A compound of formula (I) can be used in a manner known per se as the active ingredient for the production of tablets of the following composition:

| | Per tablet |
|---|---|
| Active ingredient | 200 mg |
| Microcrystalline cellulose | 155 mg |
| Corn starch | 25 mg |
| Talc | 25 mg |
| Hydroxypropylmethylcellulose | 20 mg |
| | 425 mg |

### Example 835

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

### Example 836

A compound of formula (I) can be used in a manner known per se as the active ingredient for the production of an infusion solution of the following composition:

| | |
|---|---|
| Active ingredient | 100 mg |
| Lactic acid 90% | 100 mg |

NaOH q.s. or HCl q.s. for adjustment to pH 4.0
Sodium chloride q.s. or glucose q.s. for adjustment of the
osmolality to 290 mOsm/kg

| | |
|---|---|
| Water for injection (WFI) | ad 100 ml |

### Example 837

A compound of formula (I) can be used in a manner known per se as the active ingredient for the production of an infusion solution of the following composition:

| | |
|---|---|
| Active ingredient | 100 mg |
| Hydroxypropyl-beta-cyclodextrin | 10 g |

NaOH q.s. or HCl q.s. for adjustment to pH 7.4
Sodium chloride q.s. or glucose q.s. for adjustment of the
osmolality to 290 mOsm/kg

| | |
|---|---|
| Water for injection (WFI) | ad 100 ml |

1. A compound of formula (I): 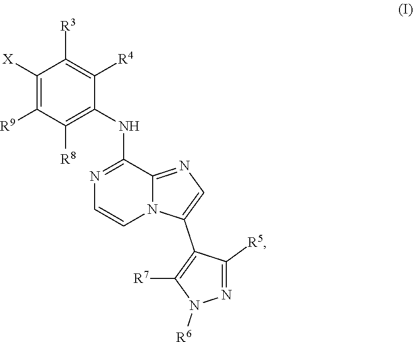
or a pharmaceutically acceptable salt thereof, wherein:
(a) X is 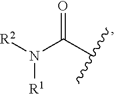
wherein:
(i) R¹ and R², taken together with the N-atom to which they are attached, form an optionally substituted C₁-C₁₉-heterocycle; or
(ii) R¹ is selected from the group consisting of:
(i) hydrogen;
(ii) optionally substituted C₁-C₆-alkyl;
(iii) optionally substituted C₂-C₆-alkenyl;
(iv) optionally substituted C₂-C₆-alkynyl;
(v) optionally substituted C₁-C₆-alkoxy;
(vi) optionally substituted C₁-C₁₉-heterocyclyl; and
(vii) optionally substituted C₃-C₁₂-cycloalkyl; and
R² is hydrogen or C₁-C₆-alkyl; or
(b) X is 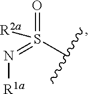
wherein:
R^{1a} is selected from the group consisting of:
(i) optionally substituted C₁-C₆-alkyl;
(ii) optionally substituted C₂-C₆-alkenyl;
(iii) optionally substituted C₂-C₆-alkynyl;
(iv) optionally substituted C₁-C₁₉-heterocyclyl; and
(v) optionally substituted C₃-C₁₂-cycloalkyl; and
R² is C₁-C₆-alkyl;
R³, R⁴, R⁸, and R⁹ are independently selected from the group consisting of hydrogen, halogen, and C₁-C₆-alkyl;
R⁵ is selected from the group consisting of halogen, cyano, C₂-C₆-alkenyl, C₂-C₆-alkynyl, halo-C₂-C₆-alkenyl, optionally substituted C₁-C₆-alkyl, C₁-C₆-alkyl-S— and a group 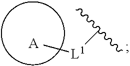
R⁶ and R⁷ are independently selected from the group consisting of hydrogen, halogen, cyano, C₂-C₆-alkenyl, C₂-C₆-alkynyl, halo-C₂-C₆-alkenyl, optionally substituted C₁-C₆-alkyl, C₁-C₆-alkyl-S— and a group 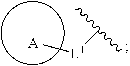
L¹ is selected from the group consisting of carbonyl, a covalent bond and C₁-C₆-alkyldiyl; and
A is selected from the group consisting of optionally substituted C₃-C₁₂-cycloalkyl, optionally substituted C₁-C₁₉-heterocyclyl, optionally substituted C₆-C₁₄-aryl, and optionally substituted C₁-C₁₃-heteroaryl.

2. The compound of formula (I) according to claim 1, or a pharmaceutically acceptable salt thereof, wherein:
(a) X is 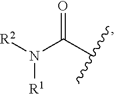
wherein:
(i) R¹ and R², taken together with the N-atom to which they are attached, form a C₁-C₁₉-heterocycle, wherein the C₁-C₁₉-heterocycle is optionally substituted with one or more, preferably 1, 2, 3 or 4 substituents selected from the group consisting of R¹⁷—C₁-C₆-alkyl-L⁴-, amino, hydroxy, and a group 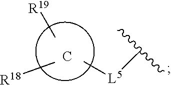
or
(ii) R¹ is selected from the group consisting of:
(i) hydrogen;
(ii) C₁-C₆-alkyl optionally substituted with one or more, preferably 1, 2, 3 or 4 substituents selected from the group consisting of C₂-C₆-alkenyl-NH—, C₁-C₆-alkoxy, C₁-C₆-alkoxycarbonyl-NH—, C₂-C₆-alkenyl-NH—, amino, halogen, cyano, hydroxy, and a group 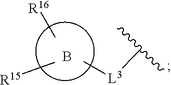
(iii) R¹⁰—C₁-C₆-alkyl-L²-C₁-C₆-alkyl-;
(iv) C₂-C₆-alkynyl;
(v) amino-C₁-C₆-alkoxy; and
(vi) a group 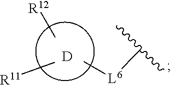
and
R² is hydrogen or C₁-C₆-alkyl; or
(b) X is 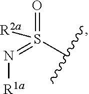
wherein:
R^{1a} is selected from the group consisting of:
(i) C₁-C₆-alkyl optionally substituted with one or more, preferably 1, 2, 3 or 4 substituents selected from the group consisting of C₂-C₆-alkenyl-NH—, C₁-C₆-alkoxy, C₁-C₆-alkoxycarbonyl-NH—, C₂-C₆-alkynyl-NH—, amino, halogen, cyano, hydroxy, and a group 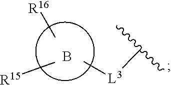
(ii) R¹⁰—C₁-C₆-alkyl-L²-C₁-C₆-alkyl-;
(iii) C₂-C₆-alkynyl; and
(iv) a group 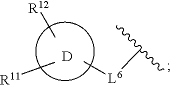
and
R^{2a} is C₁-C₆-alkyl;
R³, R⁴, R⁸, and R⁹ are independently selected from the group consisting of hydrogen, halogen, and C₁-C₆-alkyl;
R⁵ is selected from the group consisting of halogen, cyano, C₁-C₆-alkyl, C₂-C₆-alkenyl, C₂-C₆-alkynyl, halo-C₂-C₆-alkenyl, CF₃, C₁-C₆-alkyl-S—, and a group 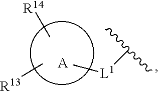
wherein said C₁-C₆-alkyl is optionally substituted with one or more, preferably 1, 2, 3 or 4 substituents independently selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R⁶ and R⁷ are independently selected from the group consisting of hydrogen, halogen, cyano, C₁-C₆-alkyl, C₂-C₆-alkenyl, C₂-C₆-alkynyl, halo-C₂-C₆-alkenyl, CF₃, C₁-C₆-alkyl-S—, and a group 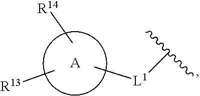
wherein said C₁-C₆-alkyl is optionally substituted with one or more, preferably 1, 2, 3 or 4 substituents independently selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R¹⁰ is selected from the group consisting of amino, C₁-C₆-alkyl-NH—, (C₁-C₆-alkyl)₂N—, C₂-C₆-alkynyl, amino-C₁-C₆-alkyl-NH—, carbamoyl, guanidine, and C₁-C₁₉-heterocyclyl, wherein the C₁-C₁₉-heterocyclyl is optionally substituted with one or two substituents selected from hydroxy and cyano-C₁-C₆-alkyl;
R¹¹ and R¹² are independently selected from the group consisting of amino, hydroxy, hydroxy-C₁-C₁₉-heterocyclyl-C(O)—, and hydroxy-C₁-C₁₉-heterocyclyl-C(O)—NH—;
R¹³ and R¹⁴ are independently selected from the group consisting of hydrogen, halogen, cyano, C₁-C₆-alkyl, amino, hydroxy, amino-C₁-C₆-alkyl, cyano-C₁-C₆-alkyl, hydroxy-C₁-C₆-alkyl, halo-C₁-C₆-alkyl, C₁-C₆-alkoxy, C₆-C₁₄-aryl-C₁-C₆-alkyl-, and C₃-C₁₂-cycloalkyl-C₁-C₆-alkyl-;
R¹⁵ and R¹⁶ are independently selected from the group consisting of hydrogen, amino, hydroxy, C₁-C₆-alkyl, and amino-C₁-C₆-alkyl;
R¹⁷ is selected from the group consisting of hydrogen, halogen, amino, C₁-C₆-alkyl-NH—, and (C₁-C₆-alkyl)₂N—;
R¹⁸ and R¹⁹ are independently selected from the group consisting of hydrogen, amino, hydroxy, and C₁-C₆-alkyl;
L¹ is selected from the group consisting of carbonyl, a covalent bond and C₁-C₆-alkyldiyl;
L² is selected from the group consisting of carbonyl, —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is selected from the group consisting of a covalent bond, carbonyl, —NH—C(O)—, and —C(O)—NH—; and
A, B, C, and D are independently selected from the group consisting of C₃-C₁₂-cycloalkyl, C₁-C₁₉-heterocyclyl, C₆-C₁₄-aryl, and C₁-C₁₃-heteroaryl.

3. The compound of formula (I) according to claim 1 or 2, wherein the compound of formula (I) is a compound of formula (II): 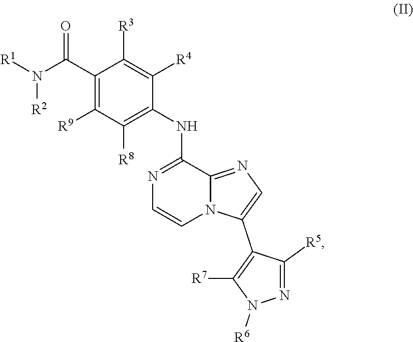
or a pharmaceutically acceptable salt thereof, wherein R¹ to R⁹ are as defined in any one of claims 1 or 2.

4. The compound of formula (II) according to claim 3, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, hydroxy, and a group 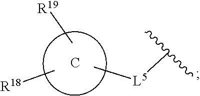
or
(ii) R¹ is selected from the group consisting of:
(i) hydrogen;
(ii) C₁-C₆-alkyl optionally substituted with one to two substituents selected from the group consisting of C₂-C₆-alkenyl-NH—, C₁-C₆-alkoxy, C₁-C₆-alkoxycarbonyl-NH—, C₂-C₆-alkynyl-NH—, amino, cyano, hydroxy, and a group 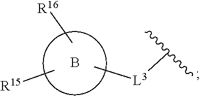
(iii) R¹⁰—C₁-C₆-alkyl-L²-C₁-C₆-alkyl-;
(iv) C₂-C₆-alkynyl;
(v) amino-C₁-C₆-alkoxy; and
(vi) a group 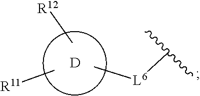
and
R² is hydrogen or C₁-C₆-alkyl.

5. The compound of formula (II) according to claim 4, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, and a group 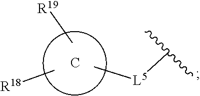
or
(ii) R¹ is selected from the group consisting of:
(i) C₁-C₆-alkyl substituted with one substituent selected from a group 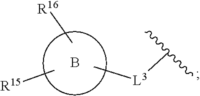
and
(ii) R¹⁰—C₁-C₆-alkyl-L²-C₁-C₆-alkyl-; and
R² is hydrogen.

6. The compound of formula (II) according to claim 5, or a pharmaceutically acceptable salt thereof, wherein:
(i) R¹ and R², taken together with the N-atom to which they are attached, form a C₁-C₁₉-heterocyclic ring selected from the group consisting of 1-piperidyl, 2,6-diazaspiro[3.3]heptan-2-yl, azetidin-1-yl, and piperazin-1-yl, wherein the C₁-C₁₉-heterocyclic ring is optionally substituted with one to two substituents selected from the group consisting of R¹⁷—CH₂-L⁴-, R¹⁷—CH(CH₃)-L⁴-, R¹⁷—(CH₂)₃-L⁴-, amino, and a group 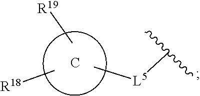
or
(ii) R¹ is selected from the group consisting of:
(i) ethylydiyl (—(CH₂)₂—) substituted with one substituent selected from a group 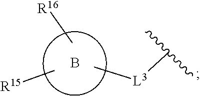
(ii) R¹⁰—(CH₂)₂-L²-(CH₂)₂—;
(iii) R¹⁰—C(CH₃)₂—CH₂-L²-(CH₂)₂—;
(iv) R¹⁰—(CH₂)₂-L²-CH₂—; and
(v) R¹⁰—CH(CH₃)—CH₂-L²-(CH₂)₂—; and
R² is hydrogen.

7. The compound of formula (I) according to any one of claims 1-6, or a pharmaceutically acceptable salt thereof, wherein R³ is C₁-C₆-alkyl or halogen.

8. The compound of formula (I) according to any one of claims 1-6, or a pharmaceutically acceptable salt thereof, wherein R³ is selected from the group consisting of methyl, ethyl, and chloro.

9. The compound of formula (I) according to any one of claims 1-8, or a pharmaceutically acceptable salt thereof, wherein R⁴ is hydrogen.

10. The compound of formula (I) according to any one of claims 1-9, or a pharmaceutically acceptable salt thereof, wherein R⁵ is halogen, cyano, C₁-C₆-alkyl-S—, halo-C₁-C₆-alkyl or a group

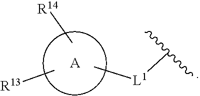

11. The compound of formula (I) according to any one of claims 1-9, or a pharmaceutically acceptable salt thereof, wherein R⁵ is halo-C₁-C₆-alkyl.

12. The compound of formula (I) according to any one of claims 1-9, or a pharmaceutically acceptable salt thereof, wherein R⁵ is CF₃ or CHF₂.

13. The compound of formula (I) according to any one of claims 1-12, or a pharmaceutically acceptable salt thereof, wherein R⁶ is selected from the group consisting of hydrogen, C₁-C₆-alkyl, C₂-C₆-alkenyl, C₂-C₆-alkynyl, halo-C₂-C₆-alkenyl, and a group 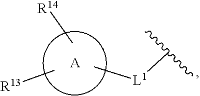
wherein the C₁-C₆-alkyl is optionally substituted with one to two substituents selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃.

14. The compound of formula (I) according to any one of claims 1-12, or a pharmaceutically acceptable salt thereof, wherein R⁶ is selected from the group consisting of hydrogen, C₁-C₆-alkyl, C₂-C₆-alkenyl, C₂-C₆-alkynyl, halo-C₂-C₆-alkenyl, and a group 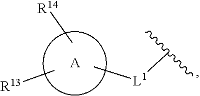
wherein the C₁-C₆-alkyl is optionally substituted with one to two substituents selected from the group consisting of C₁-C₆-alkoxy, halogen, cyano, and CF₃.

15. The compound of formula (I) according to any one of claims 1-12, or a pharmaceutically acceptable salt thereof, wherein R⁶ is selected from the group consisting of hydrogen, methyl, ethyl, propyl, vinyl, prop-2-ynyl, halo-C₂-C₆-alkenyl, and a group 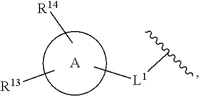
wherein said methyl, ethyl, and propyl are optionally substituted with one to two substituents selected from the group consisting of methoxy, fluoro, cyano, and CF₃.

16. The compound of formula (I) according to any one of claims 1-15, or a pharmaceutically acceptable salt thereof, wherein R⁷ is hydrogen or C₁-C₆-alkyl.

17. The compound of formula (I) according to any one of claims 1-15, or a pharmaceutically acceptable salt thereof, wherein R⁷ is hydrogen.

18. The compound of formula (I) according to any one of claims 1-17, or a pharmaceutically acceptable salt thereof, wherein R⁸ is hydrogen.

19. The compound of formula (I) according to any one of claims 1-18, or a pharmaceutically acceptable salt thereof, wherein R⁹ is hydrogen or halogen.

20. The compound of formula (I) according to any one of claims 1-18, or a pharmaceutically acceptable salt thereof, wherein R⁹ is hydrogen or fluoro.

21. The compound of formula (I) according to any one of claims 1-20, or a pharmaceutically acceptable salt thereof, wherein R¹⁰ is selected from the group consisting of amino, (C₁-C₆-alkyl)₂N—, C₂-C₆-alkynyl; amino-C₁-C₆-alkyl-NH—, carbamoyl, and guanidino.

22. The compound of formula (I) according to any one of claims 1-17, or a pharmaceutically acceptable salt thereof, wherein R¹⁰ is amino.

23. The compound of formula (I) according to any one of claims 1-22, or a pharmaceutically acceptable salt thereof, wherein R¹¹ is hydroxy-C₁-C₁₉-heterocyclyl-C(O)—.

24. The compound of formula (I) according to any one of claims 1-23, or a pharmaceutically acceptable salt thereof, wherein R¹² is selected from the group consisting of amino, and hydroxy-C₁-C₁₉-heterocyclyl-C(O)—NH—.

25. The compound of formula (I) according to any one of claims 1-24, or a pharmaceutically acceptable salt thereof, wherein R¹³ is selected from the group consisting of hydrogen, halogen, amino, C₁-C₆-alkyl, C₁-C₆-alkoxy, and C₃-C₁₂-cycloalkyl-C₁-C₆-alkyl-.

26. The compound of formula (I) according to any one of claims 1-24, or a pharmaceutically acceptable salt thereof, wherein R¹³ is hydrogen.

27. The compound of formula (I) according to any one of claims 1-26, or a pharmaceutically acceptable salt thereof, wherein R¹⁴ is selected from the group consisting of hydrogen, halogen, and C₁-C₆-alkyl.

28. The compound of formula (I) according to any one of claims 1-26, or a pharmaceutically acceptable salt thereof, wherein R¹⁴ is hydrogen.

29. The compound of formula (I) according to any one of claims 1-28, or a pharmaceutically acceptable salt thereof, wherein R¹⁵ is selected from the group consisting of hydrogen, amino, and hydroxy.

30. The compound of formula (I) according to any one of claims 1-28, or a pharmaceutically acceptable salt thereof, wherein R¹⁵ is hydrogen or amino.

31. The compound of formula (I) according to any one of claims 1-30, or a pharmaceutically acceptable salt thereof, wherein R¹⁶ is hydrogen or C₁-C₆-alkyl.

32. The compound of formula (I) according to any one of claims 1-30, or a pharmaceutically acceptable salt thereof, wherein R¹⁶ is hydrogen.

33. The compound of formula (I) according to any one of claims 1-32, or a pharmaceutically acceptable salt thereof, wherein R¹⁷ is selected from the group consisting of hydrogen, amino, and C₁-C₆-alkyl-NH—.

34. The compound of formula (I) according to any one of claims 1-32, or a pharmaceutically acceptable salt thereof, wherein R¹⁷ is amino or C₁-C₆-alkyl-NH—.

35. The compound of formula (I) according to any one of claims 1-32, or a pharmaceutically acceptable salt thereof, wherein R¹⁷ is amino or CH₃NH—.

36. The compound of formula (I) according to any one of claims 1-35, or a pharmaceutically acceptable salt thereof, wherein R¹⁸ is selected from the group consisting of hydrogen, amino, and hydroxy.

37. The compound of formula (I) according to any one of claims 1-36, or a pharmaceutically acceptable salt thereof, wherein R¹⁹ is hydrogen or C₁-C₆-alkyl.

38. The compound of formula (I) according to any one of claims 1-36, or a pharmaceutically acceptable salt thereof, wherein R¹⁹ is hydrogen.

39. The compound of formula (I) according to any one of claims 1-39, or a pharmaceutically acceptable salt thereof, wherein L¹ is a covalent bond or —CH₂—.

40. The compound of formula (I) according to any one of claims 1-38, or a pharmaceutically acceptable salt thereof, wherein L² is —NH—C(O)— or —O—.

41. The compound of formula (I) according to any one of claims 1-40, or a pharmaceutically acceptable salt thereof, wherein L³ is carbonyl or —C(O)—NH—.

42. The compound of formula (I) according to any one of claims 1-41, or a pharmaceutically acceptable salt thereof, wherein L⁴ is selected from the group consisting of a covalent bond, carbonyl, and —NH—C(O)—.

43. The compound of formula (I) according to any one of claims 1-42, or a pharmaceutically acceptable salt thereof, wherein L⁵ is carbonyl or —NH—C(O)—.

44. The compound of formula (I) according to any one of claims 1-43, or a pharmaceutically acceptable salt thereof, wherein L⁶ is a covalent bond.

45. The compound of formula (I) according to any one of claims 1-44, or a pharmaceutically acceptable salt thereof, wherein A is selected from the group consisting of C₃-C₁₂-cycloalkyl, C₁-C₁₉-heterocyclyl, and C₁-C₁₃-heteroaryl.

46. The compound of formula (I) according to any one of claims 1-44, or a pharmaceutically acceptable salt thereof, wherein A is C₃-C₁₂-cycloalkyl.

47. The compound of formula (I) according to any one of claims 1-44, or a pharmaceutically acceptable salt thereof, wherein A is cyclopropyl or cyclobutyl.

48. The compound of formula (I) according to any one of claims 1-47, or a pharmaceutically acceptable salt thereof, wherein B is C₁-C₁₉-heterocyclyl or C₃-C₁₂-cycloalkyl.

49. The compound of formula (I) according to any one of claims 1-47, or a pharmaceutically acceptable salt thereof, wherein B is selected from the group consisting of pyrrolidin-1-yl, pyrrolidin-2-yl, cyclopentyl, cyclobutyl, piperazin-1-yl, and 1-piperidyl.

50. The compound of formula (I) according to any one of claims 1-49, or a pharmaceutically acceptable salt thereof, wherein C is C₁-C₁₉-heterocyclyl or C₃-C₁₂-cycloalkyl.

51. The compound of formula (I) according to any one of claims 1-49, or a pharmaceutically acceptable salt thereof, wherein C is selected from the group consisting of pyrrolidin-1-yl, pyrrolidin-2-yl, pyrrolidin-3-yl, 1-piperidyl, 3-piperidyl, cyclobutyl, and cyclopentyl.

52. The compound of formula (I) according to any one of claims 1-51, or a pharmaceutically acceptable salt thereof, wherein D is C₁-C₁₉-heterocyclyl or C₃-C₁₂-cycloalkyl.

53. The compound of formula (I) according to any one of claims 1, 2, and 4-52, or a pharmaceutically acceptable salt thereof, wherein R^{1a} is amino-C₁-C₆-alkyl.

54. The compound of formula (I) according to claim 1 or 2, or a pharmaceutically acceptable salt thereof, wherein:
(a) X is 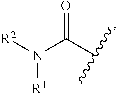
wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, hydroxy, and a group 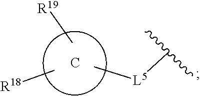
or
(ii) R¹ is selected from the group consisting of:
(i) hydrogen;
(ii) C₁-C₆-alkyl optionally substituted with one to two substituents selected from the group consisting of C₂-C₆-alkenyl-NH—, C₁-C₆-alkoxy, C₁-C₆-alkoxycarbonyl-NH—, C₂-C₆-alkynyl-NH—, amino, cyano, hydroxy, and a group 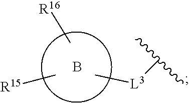
(iii) R¹⁰—C₁-C₆-alkyl-L²-C₁-C₆-alkyl-;
(iv) C₂-C₆-alkynyl;
(v) amino-C₁-C₆-alkoxy; and
(vi) a group 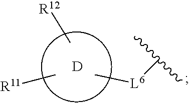
and
R² is hydrogen or C₁-C₆-alkyl; or
(b) X is 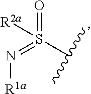
wherein R^{1a} is amino-C₁-C₆-alkyl and R^{2a} is C₁-C₆-alkyl;R³ is selected from the group consisting of hydrogen, halogen, and C₁-C₆-alkyl;
R⁴ is hydrogen;
R⁵ is halogen, cyano, C₁-C₆-alkyl-S—, halo-C₁-C₆-alkyl or a group 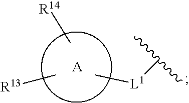
R⁶ is selected from the group consisting of hydrogen, C₁-C₆-alkyl, C₂-C₆-alkenyl,
C₂-C₆-alkynyl, halo-C₂-C₆-alkenyl, and a group 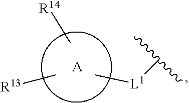
wherein the C₁-C₆-alkyl is optionally substituted with one to two substituents selected from the group consisting of C₁-C₆-alkoxy, halo-C₁-C₆-alkoxy, C₁-C₆-alkoxycarbonyl, hydroxy, halogen, (C₁-C₆-alkyl)₂N—, C₁-C₆-alkyl-NH—, amino, carbamoyl, carboxy, cyano, and CF₃;
R⁷ is hydrogen or C₁-C₆-alkyl;
R⁸ is hydrogen;
R⁹ is selected from the group consisting of hydrogen, halogen, and C₁-C₆-alkyl;
R¹⁰ is selected from the group consisting of amino, (C₁-C₆-alkyl)₂N—, C₂-C₆-alkynyl; amino-C₁-C₆-alkyl-NH—, carbamoyl, and guanidino;
R¹¹ is hydroxy-C₁-C₁₉-heterocyclyl-C(O)—;
R¹² is selected from the group consisting of amino, and hydroxy-C₁-C₁₉-heterocyclyl-C(O)—NH—;
R¹³ is selected from the group consisting of hydrogen, halogen, amino, C₁-C₆-alkyl, C₁-C₆-alkoxy, and C₃-C₁₂-cycloalkyl-C₁-C₆-alkyl-;
R¹⁴ is selected from the group consisting of hydrogen, halogen, and C₁-C₆-alkyl;
R¹⁵ is selected from the group consisting of hydrogen, amino, and hydroxy;
R¹⁶ is hydrogen or C₁-C₆-alkyl;
R¹⁷ is selected from the group consisting of hydrogen, amino, and C₁-C₆-alkyl-NH—;
R¹⁸ is selected from the group consisting of hydrogen, amino, and hydroxy;
R¹⁹ is hydrogen or C₁-C₆-alkyl;
L¹ is selected from the group consisting of carbonyl, a covalent bond and C₁-C₆-alkyldiyl;
L² is selected from the group consisting of —C(O)—NH—, —NH—C(O)—, —NH—C(O)—NH—, —CH(NH₂)—C(O)—NH—, —NH—, and —O—;
L³ is selected from the group consisting of a covalent bond, carbonyl, —NH—C(O)—, and —C(O)—NH—;
L⁴ is selected from the group consisting of a covalent bond, carbonyl, —NH—C(O)—, —C(O)—NH—, and —NH—C(O)—CH₂—;
L⁵ is selected from the group consisting of —CH₂—C(O)—, —C(O)—CH₂—, —CH₂—NH—C(O)—, —NH—C(O)—CH₂—, carbonyl, —NH—C(O)—, —C(O)—NH—, and —NH—C(O)—NH—;
L⁶ is a covalent bond;
A is selected from the group consisting of C₃-C₁₂-cycloalkyl, C₁-C₁₉-heterocyclyl, and C₁-C₁₃-heteroaryl;
B is C₁-C₁₉-heterocyclyl or C₃-C₁₂-cycloalkyl;
C is C₁-C₁₉-heterocyclyl or C₃-C₁₂-cycloalkyl; and
D is C₁-C₁₉-heterocyclyl or C₃-C₁₂-cycloalkyl.

55. The compound of formula (II) according to claim 3, or a pharmaceutically acceptable salt thereof, wherein:
(i) R¹ and R², taken together with the N-atom to which they are attached, form a C₁-C₁₉-heterocyclic ring, wherein the C₁-C₁₉-heterocyclic ring is optionally substituted with one to two substituents selected from the group consisting of R¹⁷—C₁-C₆-alkyl-L⁴-, amino, and a group 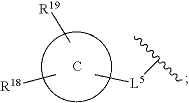
or
(ii) R¹ is selected from the group consisting of:
(i) C₁-C₆-alkyl substituted with one substituent selected from a group 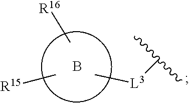
and
(ii) R¹⁰—C₁-C₆-alkyl-L²-C₁-C₆-alkyl-; and
R² is hydrogen;
R³ is C₁-C₆-alkyl or halogen;
R⁴ is hydrogen;
R⁵ is halo-C₁-C₆-alkyl;
R⁶ is selected from the group consisting of hydrogen, C₁-C₆-alkyl, C₂-C₆-alkenyl, C₂-C₆-alkynyl, halo-C₂-C₆-alkenyl, and a group 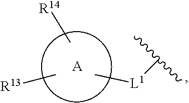
wherein
the C₁-C₆-alkyl is optionally substituted with one to two substituents selected from the group consisting of C₁-C₆-alkoxy, halogen, cyano, and CF₃;
R⁷ is hydrogen;
R⁸ is hydrogen;
R⁹ is hydrogen or halogen;
R¹⁰ is amino;
R¹³ is hydrogen;
R¹⁴ is hydrogen;
R¹⁵ is hydrogen or amino;
R¹⁶ is hydrogen;
R¹⁷ is amino or C₁-C₆-alkyl-NH—;
R¹⁸ is selected from the group consisting of hydrogen, amino, and hydroxy;
R¹⁹ is hydrogen;
L¹ is a covalent bond or —CH₂—;
L² is —NH—C(O)— or —O—;
L³ is carbonyl or —C(O)—NH—;
L⁴ is selected from the group consisting of a covalent bond, carbonyl, and —NH—C(O);
L⁵ is carbonyl or —NH—C(O)—;
A is C₃-C₁₂-cycloalkyl;
B is C₁-C₁₉-heterocyclyl or C₃-C₁₂-cycloalkyl; and
C is C₁-C₁₉-heterocyclyl or C₃-C₁₂-cycloalkyl.

56. The compound of formula (II) according to claim 55, or a pharmaceutically acceptable salt thereof, wherein:
(i) R¹ and R², taken together with the N-atom to which they are attached, form a C₁-C₁₉-heterocyclic ring selected from the group consisting of 1-piperidyl, 2,6-diazaspiro[3.3]heptan-2-yl, azetidin-1-yl, and piperazin-1-yl, wherein the C₁-C₁₉-heterocyclic ring is optionally substituted with one to two substituents selected from the group consisting of R¹⁷—CH₂-L⁴-, R¹⁷—CH(CH₃)-L⁴-, R¹⁷—(CH₂)₃-L⁴-, amino, and a group 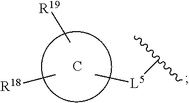
or
(ii) R¹ is selected from the group consisting of:
(i) ethylydiyl (—(CH₂)₂—) substituted with one substituent selected from a group 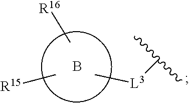
(ii) R¹⁰—(CH₂)₂-L¹-(CH₂)₂—;
(iii) R¹⁰—C(CH₃)₂—CH₂-L¹-(CH₂)₂—;
(iv) R¹⁰—(CH₂)₂-L¹-CH₂—; and
(v) R¹⁰—CH(CH₃)—CH₂-L¹-(CH₂)₂—; and
R² is hydrogen;
R³ is selected from the group consisting of methyl, ethyl, and chloro;
R⁴ is hydrogen;
R⁵ is CF₃ or CHF₂;
R⁶ is selected from the group consisting of hydrogen, methyl, ethyl, propyl, vinyl, prop-2-ynyl, halo-C₂-C₆-alkenyl, and a group 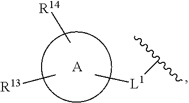
wherein said methyl, ethyl, and propyl are optionally substituted with one to two substituents selected from the group consisting of methoxy, fluoro, cyano, and CF₃;
R⁷ is hydrogen;
R⁸ is hydrogen;
R⁹ is hydrogen or fluoro;
R¹⁰ is amino;
R¹³ is hydrogen;
R¹⁴ is hydrogen;
R¹⁵ is hydrogen or amino;
R¹⁶ is hydrogen;
R¹⁷ is amino or CH₃NH—;
R¹⁸ is selected from the group consisting of hydrogen, amino, and hydroxy;
R¹⁹ is hydrogen;
L¹ is a covalent bond or —CH₂—;
L² is —NH—C(O)— or —O—;
L³ is carbonyl or —C(O)—NH—;
L⁴ is selected from the group consisting of a covalent bond, carbonyl, and —NH—C(O)—;
L⁵ is carbonyl or —NH—C(O)—;
A is cyclopropyl or cyclobutyl;
B is selected from the group consisting of pyrrolidin-1-yl, pyrrolidin-2-yl, cyclopentyl, cyclobutyl, piperazin-1-yl, and 1-piperidyl; and
C is selected from the group consisting of pyrrolidin-1-yl, pyrrolidin-2-yl, pyrrolidin-3-yl, 1-piperidyl, 3-piperidyl, cyclobutyl, and cyclopentyl.

57. The compound of formula (I) according to claim 1, or a pharmaceutically acceptable salt thereof, wherein the compound of formula (I) is selected from:
N-[2-(2-aminoethoxy)ethyl]-2-ethyl-4-[[3-[1-methyl-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(pyrimidin-4-ylmethyl)-3-(trifluoromethyl)pyrazol-4-yl]imidazo[1,2-α]pyrazin-8-yl]amino]benzamide;
(2S)—N-[2-[[4-[[3-[1-(cyanomethyl)-3-(trifluoromethyl)pyrazol-4-yl]imidazo[1,2-α]pyrazin-8-yl]amino]-2-ethylbenzoyl]amino]ethyl]pyrrolidine-2-carboxamide;
4-[[3-[1-(cyanomethyl)-3-(trifluoromethyl)pyrazol-4-yl]imidazo[1,2-α]pyrazin-8-yl]amino]-2-fluoro-6-methyl-N-prop-2-ynylbenzamide;
[2-ethyl-4-[[3-[1-prop-2-ynyl-3-(trifluoromethyl)pyrazol-4-yl]imidazo[1,2-α]pyrazin-8-yl]amino]phenyl]-[4-(piperidine-4-carbonyl)piperazin-1-yl]methanone;
2-amino-1-[4-[2-chloro-4-[[3-[1-(2,2-difluoroethyl)-3-(trifluoromethyl)pyrazol-4-yl]imidazo[1,2-α]pyrazin-8-yl]amino]benzoyl]piperazin-1-yl]ethanone;
N-[3-[4-(3-aminopropylamino)butylamino]propyl]-2-ethyl-4-[[3-[1-(pyridin-3-ylmethyl)-3-(trifluoromethyl)pyrazol-4-yl]imidazo[1,2-α]pyrazin-8-yl]amino]benzamide;
N-[2-(2-aminoethylamino)-2-oxoethyl]-2-chloro-4-[[3-[1-(2-fluoroethyl)-3-(trifluoromethyl)pyrazol-4-yl]imidazo[1,2-α]pyrazin-8-yl]amino]benzamide;
[2-chloro-4-[difluoromethyl-[3-[3-(trifluoromethyl)-1H-pyrazol-4-yl]imidazo[1,2-α]pyrazin-8-yl]amino]phenyl]-piperazin-1-ylmethanone;
2-chloro-4-[[3-[1-(difluoromethyl)-3-(trifluoromethyl)pyrazol-4-yl]imidazo[1,2-α]pyrazin-8-yl]amino]-N-methylbenzamide;
1-[1-[2-chloro-4-[[3-[1-(cyanomethyl)-3-(trifluoromethyl)pyrazol-4-yl]imidazo[1,2-α]pyrazin-8-yl]amino]benzoyl]piperidin-4-yl]-3-[(3S)-pyrrolidin-3-yl]urea;
2-[4-[8-[4-[4-(3-aminocyclobutanecarbonyl)piperazine-1-carbonyl]-3-chloroanilino]imidazo[1,2-α]pyrazin-3-yl]-3-(trifluoromethyl)pyrazol-1-yl]acetonitrile;
N-[3-[[(2S)-2-amino-3-carbamimidamidopropanoyl]amino]propyl]-2-ethyl-4-[[3-[3-(trifluoromethyl)-1H-pyrazol-4-yl]imidazo[1,2-α]pyrazin-8-yl]amino]benzamide;
2-[4-[8-[3-chloro-4-[4-hydroxy-4-(methylaminomethyl)piperidine-1-carbonyl]anilino]imidazo[1,2-α]pyrazin-3-yl]-3-(trifluoromethyl)pyrazol-1-yl]acetonitrile;
2-ethyl-N-methyl-4-[[3-[1-(1H-pyrazol-3-ylmethyl)-3-(trifluoromethyl)pyrazol-4-yl]imidazo[1,2-α]pyrazin-8-yl]amino]benzamide;
N-[2-(2-aminoethoxy)ethyl]-2-methyl-4-[[3-[1-prop-2-ynyl-3-(trifluoromethyl)pyrazol-4-yl]imidazo[1,2-α]pyrazin-8-yl]amino]benzamide;
[4-(3-aminocyclobutanecarbonyl)piperazin-1-yl]-[2-chloro-4-[[3-[3-(trifluoromethyl)-1H-pyrazol-4-yl]imidazo[1,2-α]pyrazin-8-yl]amino]phenyl]methanone;
N-[2-(2-aminoethoxy)ethyl]-4-[[3-[1-(cyanomethyl)-3-(difluoromethyl)pyrazol-4-yl]imidazo[1,2-α]pyrazin-8-yl]amino]-2-ethylbenzamide;
N-[2-(2-aminoethoxy)ethyl]-4-[[3-[3-(difluoromethyl)-1-(methoxymethyl)pyrazol-4-yl]imidazo[1,2-α]pyrazin-8-yl]amino]-2-ethylbenzamide;
N-[2-(2-aminoethoxy)ethyl]-4-[[3-[1-(2,2-difluoroethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-(methoxymethyl)-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[3-(trifluoromethyl)-1H-pyrazol-4-yl]imidazo[1,2-α]pyrazin-8-yl]amino]benzamide;
N-[2-(2-aminoethoxy)ethyl]-2-ethyl-4-[[3-[5-methyl-3-(trifluoromethyl)-1H-pyrazol-4-yl]imidazo[1,2-α]pyrazin-8-yl]amino]benzamide;
N-[2-(2-aminoethoxy)ethyl]-2-ethyl-4-[[3-[1-propyl-3-(trifluoromethyl)pyrazol-4-yl]imidazo[1,2-α]pyrazin-8-yl]amino]benzamide;
N-[2-(2-aminoethoxy)ethyl]-4-[[3-[1-cyclopentyl-3-(trifluoromethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-propan-2-yl-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(3-methylbutyl)-3-(trifluoromethyl)pyrazol-4-yl]imidazo[1,2-α]pyrazin-8-yl]amino]benzamide;
N-[2-(2-aminoethoxy)ethyl]-4-[[3-[1-(cyanomethyl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(cyclopropylmethyl)-3-(trifluoromethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-ethyl-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[3-(trifluoromethyl)-1-(3,3,3-trifluoropropyl)pyrazol-4-yl]imidazo[1,2-α]pyrazin-8-yl]amino]benzamide;
N-[2-(2-aminoethoxy)ethyl]-4-[[3-[1-(2-cyanoethyl)-3-(trifluoromethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-(2-methoxyethyl)-3-(trifluoromethyl)pyrazol-4-yl]imidazo[1,2-α]pyrazin-8-yl]amino]benzamide;
(2S)—N-[2-[[4-[[3-[1-(2,2-difluoroethyl)-3-(trifluoromethyl)pyrazol-4-yl]imidazo[1,2-α]pyrazin-8-yl]amino]-2-ethylbenzoyl]amino]ethyl]pyrrolidine-2-carboxamide;
(2S)—N-[2-[[4-[[3-[1-(difluoromethyl)-3-(trifluoromethyl)pyrazol-4-yl]imidazo[1,2-α]pyrazin-8-yl]amino]-2-ethylbenzoyl]amino]ethyl]pyrrolidine-2-carboxamide;
4-[[3-[1-(2,2-difluoroethyl)-3-(trifluoromethyl)pyrazol-4-yl]imidazo[1,2-α]pyrazin-8-yl]amino]-2-fluoro-6-methyl-N-prop-2-ynylbenzamide;
4-[[3-[1-[[3-(cyclopropylmethyl)-1,2,4-oxadiazol-5-yl]methyl]-3-(trifluoromethyl)pyrazol-4-yl]imidazo[1,2-α]pyrazin-8-yl]amino]-2-fluoro-6-methyl-N-prop-2-ynylbenzamide;
(2S)—N-[2-[[2-ethyl-4-[[3-[3-(trifluoromethyl)-1H-pyrazol-4-yl]imidazo[1,2-α]pyrazin-8-yl]amino]benzoyl]amino]ethyl]pyrrolidine-2-carboxamide;
2-fluoro-6-methyl-N-prop-2-ynyl-4-[[3-[1-prop-2-ynyl-3-(trifluoromethyl)pyrazol-4-yl]imidazo[1,2-α]pyrazin-8-yl]amino]benzamide;
2-fluoro-6-methyl-4-[[3-[1-prop-2-enyl-3-(trifluoromethyl)pyrazol-4-yl]imidazo[1,2-α]pyrazin-8-yl]amino]-N-prop-2-ynylbenzamide;
2-fluoro-4-[[3-[1-(2-fluoroethyl)-3-(trifluoromethyl)pyrazol-4-yl]imidazo[1,2-α]pyrazin-8-yl]amino]-6-methyl-N-prop-2-ynylbenzamide;
2-fluoro-6-methyl-N-prop-2-ynyl-4-[[3-[3-(trifluoromethyl)-1H-pyrazol-4-yl]imidazo[1,2-α]pyrazin-8-yl]amino]benzamide;
N-[2-(2-amino-2-methylpropoxy)ethyl]-2-ethyl-4-[[3-[1-prop-2-ynyl-3-(trifluoromethyl)pyrazol-4-yl]imidazo[1,2-α]pyrazin-8-yl]amino]benzamide;
N-[2-(2-amino-2-methylpropoxy)ethyl]-2-ethyl-4-[[3-[1-prop-2-enyl-3-(trifluoromethyl)pyrazol-4-yl]imidazo[1,2-α]pyrazin-8-yl]amino]benzamide;
[4-(aminomethyl)piperidin-1-yl]-[2-ethyl-4-[[3-[1-prop-2-ynyl-3-(trifluoromethyl)pyrazol-4-yl]imidazo[1,2-α]pyrazin-8-yl]amino]phenyl]methanone;
N-(3-aminopropyl)-2-ethyl-4-[[3-[1-prop-2-ynyl-3-(trifluoromethyl)pyrazol-4-yl]imidazo[1,2-α]pyrazin-8-yl]amino]benzamide;
N-(3-aminopropyl)-2-ethyl-4-[[3-[1-prop-2-enyl-3-(trifluoromethyl)pyrazol-4-yl]imidazo[1,2-α]pyrazin-8-yl]amino]benzamide;
N-[2-(2-aminoethoxy)ethyl]-4-[[3-[1-(2,2-difluoroethyl)-3-(trifluoromethyl)pyrazol-4-yl]imidazo[1,2-α]pyrazin-8-yl]amino]-2-fluoro-6-methylbenzamide;
N-[2-(2-amino-2-methylpropoxy)ethyl]-4-[[3-[1-(2,2-difluoroethyl)-3-(trifluoromethyl)pyrazol-4-yl]imidazo[1,2-α]pyrazin-8-yl]amino]-2-ethylbenzamide;
N-[2-(2-aminoethoxy)ethyl]-2-fluoro-6-methyl-4-[[3-[1-prop-2-enyl-3-(trifluoromethyl)pyrazol-4-yl]imidazo[1,2-α]pyrazin-8-yl]amino]benzamide;
N-(3-aminopropyl)-4-[[3-[1-(2,2-difluoroethyl)-3-(trifluoromethyl)pyrazol-4-yl]imidazo[1,2-α]pyrazin-8-yl]amino]-2-ethylbenzamide;
[4-(aminomethyl)piperidin-1-yl]-[2-ethyl-4-[[3-[1-prop-2-enyl-3-(trifluoromethyl)pyrazol-4-yl]imidazo[1,2-α]pyrazin-8-yl]amino]phenyl]methanone;
N-[2-(2-amino-2-methylpropoxy)ethyl]-2-ethyl-4-[[3-[1-(2-fluoroethyl)-3-(trifluoromethyl)pyrazol-4-yl]imidazo[1,2-α]pyrazin-8-yl]amino]benzamide;
N-[(2S)-1-aminopropan-2-yl]-2-ethyl-4-[[3-[1-prop-2-ynyl-3-(trifluoromethyl)pyrazol-4-yl]imidazo[1,2-α]pyrazin-8-yl]amino]benzamide;
N-(3-aminopropyl)-2-ethyl-4-[[3-[1-(2-fluoroethyl)-3-(trifluoromethyl)pyrazol-4-yl]imidazo[1,2-α]pyrazin-8-yl]amino]benzamide;
N-[(2S)-1-aminopropan-2-yl]-4-[[3-[1-(2,2-difluoroethyl)-3-(trifluoromethyl)pyrazol-4-yl]imidazo[1,2-α]pyrazin-8-yl]amino]-2-ethylbenzamide;
[4-(aminomethyl)piperidin-1-yl]-[4-[[3-[1-(2,2-difluoroethyl)-3-(trifluoromethyl)pyrazol-4-yl]imidazo[1,2-α]pyrazin-8-yl]amino]-2-ethylphenyl]methanone;
N-[2-(2-aminoethoxy)ethyl]-2-fluoro-6-methyl-4-[[3-[1-prop-2-ynyl-3-(trifluoromethyl)pyrazol-4-yl]imidazo[1,2-α]pyrazin-8-yl]amino]benzamide;
N-[2-(2-amino-2-methylpropoxy)ethyl]-4-[[3-[1-(cyanomethyl)-3-(trifluoromethyl)pyrazol-4-yl]imidazo[1,2-α]pyrazin-8-yl]amino]-2-ethylbenzamide;
N-[2-(2-aminoethoxy)ethyl]-2-fluoro-4-[[3-[1-(2-fluoroethyl)-3-(trifluoromethyl)pyrazol-4-yl]imidazo[1,2-α]pyrazin-8-yl]amino]-6-methylbenzamide;
[4-(aminomethyl)piperidin-1-yl]-[2-ethyl-4-[[3-[1-(2-fluoroethyl)-3-(trifluoromethyl)pyrazol-4-yl]imidazo[1,2-α]pyrazin-8-yl]amino]phenyl]methanone;
N-[(2S)-1-aminopropan-2-yl]-2-ethyl-4-[[3-[1-prop-2-enyl-3-(trifluoromethyl)pyrazol-4-yl]imidazo[1,2-α]pyrazin-8-yl]amino]benzamide;
[2-ethyl-4-[[3-[1-prop-2-enyl-3-(trifluoromethyl)pyrazol-4-yl]imidazo[1,2-α]pyrazin-8-yl]amino]phenyl]-[4-(piperidine-4-carbonyl)piperazin-1-yl]methanone;
N-[(2S)-1-aminopropan-2-yl]-2-ethyl-4-[[3-[1-(2-fluoroethyl)-3-(trifluoromethyl)pyrazol-4-yl]imidazo[1,2-α]pyrazin-8-yl]amino]benzamide;
2-fluoro-6-methyl-4-[[3-[1-prop-2-enyl-3-(trifluoromethyl)pyrazol-4-yl]imidazo[1,2-α]pyrazin-8-yl]amino]-N-[3-(prop-2-ynylamino)propyl]benzamide;
[2-fluoro-6-methyl-4-[[3-[1-prop-2-ynyl-3-(trifluoromethyl)pyrazol-4-yl]imidazo[1,2-α]pyrazin-8-yl]amino]phenyl]-piperazin-1-ylmethanone;
4-[[3-[1-(2,2-difluoroethyl)-3-(trifluoromethyl)pyrazol-4-yl]imidazo[1,2-α]pyrazin-8-yl]amino]-2-fluoro-6-methyl-N-[3-(prop-2-ynylamino)propyl]benzamide;
[2-fluoro-6-methyl-4-[[3-[1-prop-2-enyl-3-(trifluoromethyl)pyrazol-4-yl]imidazo[1,2-α]pyrazin-8-yl]amino]phenyl]-piperazin-1-ylmethanone;
[4-[[3-[1-(2,2-difluoroethyl)-3-(trifluoromethyl)pyrazol-4-yl]imidazo[1,2-α]pyrazin-8-yl]amino]-2-ethylphenyl]-[4-(piperidine-4-carbonyl)piperazin-1-yl]methanone;
[2-ethyl-4-[[3-[1-(2-fluoroethyl)-3-(trifluoromethyl)pyrazol-4-yl]imidazo[1,2-α]pyrazin-8-yl]amino]phenyl]-[4-(piperidine-4-carbonyl)piperazin-1-yl]methanone;
2-fluoro-6-methyl-N-[3-(prop-2-ynylamino)propyl]-4-[[3-[1-prop-2-ynyl-3-(trifluoromethyl)pyrazol-4-yl]imidazo[1,2-α]pyrazin-8-yl]amino]benzamide;
[2-fluoro-4-[[3-[1-(2-fluoroethyl)-3-(trifluoromethyl)pyrazol-4-yl]imidazo[1,2-α]pyrazin-8-yl]amino]-6-methylphenyl]-piperazin-1-ylmethanone;
2-fluoro-4-[[3-[1-(2-fluoroethyl)-3-(trifluoromethyl)pyrazol-4-yl]imidazo[1,2-α]pyrazin-8-yl]amino]-6-methyl-N-[3-(prop-2-ynylamino)propyl]benzamide;
N-[2-(2-aminoethoxy)ethyl]-4-[[3-[1-(cyanomethyl)-3-(trifluoromethyl)pyrazol-4-yl]imidazo[1,2-α]pyrazin-8-yl]amino]-2-fluoro-6-methylbenzamide;
4-[[3-[1-(cyanomethyl)-3-(trifluoromethyl)pyrazol-4-yl]imidazo[1,2-α]pyrazin-8-yl]amino]-2-fluoro-6-methyl-N-[3-(prop-2-ynylamino)propyl]benzamide;
2-[4-[8-[3-fluoro-5-methyl-4-(piperazine-1-carbonyl)anilino]imidazo[1,2-α]pyrazin-3-yl]-3-(trifluoromethyl)pyrazol-1-yl]acetonitrile;
2-amino-1-[4-[2-chloro-4-[[3-[1-(difluoromethyl)-3-(trifluoromethyl)pyrazol-4-yl]imidazo[1,2-α]pyrazin-8-yl]amino]benzoyl]piperazin-1-yl]ethanone;
[2-chloro-4-[[3-[1-(difluoromethyl)-3-(trifluoromethyl)pyrazol-4-yl]imidazo[1,2-α]pyrazin-8-yl]amino]phenyl]-(2,6-diazaspiro[3.3]heptan-2-yl)methanone;
N-[2-(2-aminoethoxy)ethyl]-2-fluoro-6-methyl-4-[[3-[3-(trifluoromethyl)-1H-pyrazol-4-yl]imidazo[1,2-α]pyrazin-8-yl]amino]benzamide;
N-(5-aminopentyl)-2-fluoro-6-methyl-4-[[3-[3-(trifluoromethyl)-1H-pyrazol-4-yl]imidazo[1,2-α]pyrazin-8-yl]amino]benzamide;
N-[3-[4-(3-aminopropylamino)butylamino]propyl]-2-ethyl-4-[[3-[1-[(2-methyl-1,3-thiazol-4-yl)methyl]-3-(trifluoromethyl)pyrazol-4-yl]imidazo[1,2-α]pyrazin-8-yl]amino]benzamide;
N-[(2R)-1-aminopropan-2-yl]-2-fluoro-6-methyl-4-[[3-[3-(trifluoromethyl)-1H-pyrazol-4-yl]imidazo[1,2-α]pyrazin-8-yl]amino]benzamide;
N-[3-[4-(3-aminopropylamino)butylamino]propyl]-2-ethyl-4-[[3-[1-[(1-methyl)imidazol-2-yl)methyl]-3-(trifluoromethyl)pyrazol-4-yl]imidazo[1,2-α]pyrazin-8-yl]amino]benzamide;
2-fluoro-6-methyl-N-[3-(prop-2-ynylamino)propyl]-4-[[3-[3-(trifluoromethyl)-1H-pyrazol-4-yl]imidazo[1,2-α]pyrazin-8-yl]amino]benzamide;
[2-fluoro-6-methyl-4-[[3-[3-(trifluoromethyl)-1H-pyrazol-4-yl]imidazo[1,2-α]pyrazin-8-yl]amino]phenyl]-piperazin-1-ylmethanone;
N-(3-amino-1-bicyclo[1.1.1]pentanyl)-2-chloro-4-[[3-[1-(2,2-difluoroethyl)-3-(trifluoromethyl)pyrazol-4-yl]imidazo[1,2-α]pyrazin-8-yl]amino]benzamide;
N-(3-amino-1-bicyclo[1.1.1]pentanyl)-2-chloro-4-[[3-[1-(cyanomethyl)-3-(trifluoromethyl)pyrazol-4-yl]imidazo[1,2-α]pyrazin-8-yl]amino]benzamide;
N-[2-(2-aminoethylamino)-2-oxoethyl]-4-[[3-[1-but-2-ynyl-3-(trifluoromethyl)pyrazol-4-yl]imidazo[1,2-α]pyrazin-8-yl]amino]-2-chlorobenzamide;
N-[(2R)-1-(4-aminopiperidin-1-yl)-1-oxopropan-2-yl]-2-chloro-4-[[3-[3-(trifluoromethyl)-1H-pyrazol-4-yl]imidazo[1,2-α]pyrazin-8-yl]amino]benzamide;
2-chloro-N-[(2R)-1-oxo-1-piperazin-1-ylpropan-2-yl]-4-[[3-[3-(trifluoromethyl)-1H-pyrazol-4-yl]imidazo[1,2-α]pyrazin-8-yl]amino]benzamide;
N-[(2R)-1-[(3-aminocyclobutyl)amino]-1-oxopropan-2-yl]-2-chloro-4-[[3-[3-(trifluoromethyl)-1H-pyrazol-4-yl]imidazo[1,2-α]pyrazin-8-yl]amino]benzamide;
2-chloro-N-[(2R)-1-oxo-1-[[(3R)-pyrrolidin-3-yl]amino]propan-2-yl]-4-[[3-[3-(trifluoromethyl)-1H-pyrazol-4-yl]imidazo[1,2-α]pyrazin-8-yl]amino]benzamide;
N-[(2R)-1-[(3S)-3-aminopyrrolidin-1-yl]-1-oxopropan-2-yl]-2-chloro-4-[[3-[3-(trifluoromethyl)-1H-pyrazol-4-yl]imidazo[1,2-α]pyrazin-8-yl]amino]benzamide;
N-[(2R)-1-(2-aminoethylamino)-1-oxopropan-2-yl]-2-chloro-4-[[3-[3-(trifluoromethyl)-1H-pyrazol-4-yl]imidazo[1,2-α]pyrazin-8-yl]amino]benzamide;
2-chloro-N-[(2R)-1-oxo-1-[[(3S)-pyrrolidin-3-yl]amino]propan-2-yl]-4-[[3-[3-(trifluoromethyl)-1H-pyrazol-4-yl]imidazo[1,2-α]pyrazin-8-yl]amino]benzamide;
N-[(2R)-1-[(3R)-3-aminopyrrolidin-1-yl]-1-oxopropan-2-yl]-2-chloro-4-[[3-[3-(trifluoromethyl)-1H-pyrazol-4-yl]imidazo[1,2-α]pyrazin-8-yl]amino]benzamide;
2-chloro-N-[(2R)-1-[2-(dimethylamino)ethylamino]-1-oxopropan-2-yl]-4-[[3-[3-(trifluoromethyl)-1H-pyrazol-4-yl]imidazo[1,2-α]pyrazin-8-yl]amino]benzamide;
N-[(2R)-1-(3-aminoazetidin-1-yl)-1-oxopropan-2-yl]-2-chloro-4-[[3-[3-(trifluoromethyl)-1H-pyrazol-4-yl]imidazo[1,2-α]pyrazin-8-yl]amino]benzamide;
2-[4-[8-[4-(3-aminoazetidine-1-carbonyl)-3-chloroanilino]imidazo[1,2-α]pyrazin-3-yl]-3-(trifluoromethyl)pyrazol-1-yl]acetonitrile;
(3-aminoazetidin-1-yl)-[2-chloro-4-[[3-[1-(2,2-difluoroethyl)-3-(trifluoromethyl)pyrazol-4-yl]imidazo[1,2-α]pyrazin-8-yl]amino]phenyl]methanone;
2-[4-[8-[3-chloro-4-(2,6-diazaspiro[3.3]heptane-2-carbonyl)anilino]imidazo[1,2-α]pyrazin-3-yl]-3-(trifluoromethyl)pyrazol-1-yl]acetonitrile;
(4-aminopiperidin-1-yl)-[2-chloro-4-[[3-[1-(2,2-difluoroethyl)-3-(trifluoromethyl)pyrazol-4-yl]imidazo[1,2-α]pyrazin-8-yl]amino]phenyl]methanone;
[2-chloro-4-[[3-[1-(difluoromethyl)-3-(trifluoromethyl)pyrazol-4-yl]imidazo[1,2-α]pyrazin-8-yl]amino]phenyl]-[4-[(2S)-pyrrolidine-2-carbonyl]piperazin-1-yl]methanone;
N-(3-amino-1-bicyclo[1.1.1]pentanyl)-2-chloro-4-[[3-[3-(trifluoromethyl)-1H-pyrazol-4-yl]imidazo[1,2-α]pyrazin-8-yl]amino]benzamide;
N-[(2R)-1-aminopropan-2-yl]-2-ethyl-4-[[3-[1-prop-2-ynyl-3-(trifluoromethyl)pyrazol-4-yl]imidazo[1,2-α]pyrazin-8-yl]amino]benzamide;
N-[(2R)-1-aminopropan-2-yl]-4-[[3-[1-(2,2-difluoroethyl)-3-(trifluoromethyl)pyrazol-4-yl]imidazo[1,2-α]pyrazin-8-yl]amino]-2-ethylbenzamide;
N-[(2R)-1-aminopropan-2-yl]-2-ethyl-4-[[3-[1-prop-2-enyl-3-(trifluoromethyl)pyrazol-4-yl]imidazo[1,2-α]pyrazin-8-yl]amino]benzamide;
N-[(2R)-1-aminopropan-2-yl]-4-[[3-[1-(cyanomethyl)-3-(trifluoromethyl)pyrazol-4-yl]imidazo[1,2-α]pyrazin-8-yl]amino]-2-ethylbenzamide;
N-[(2R)-1-aminopropan-2-yl]-2-ethyl-4-[[3-[1-(2-fluoroethyl)-3-(trifluoromethyl)pyrazol-4-yl]imidazo[1,2-α]pyrazin-8-yl]amino]benzamide;
[2-chloro-4-[[3-[3-(trifluoromethyl)-1H-pyrazol-4-yl]imidazo[1,2-α]pyrazin-8-yl]amino]phenyl]-(2,6-diazaspiro[3.3]heptan-2-yl)methanone;
N,2-diethyl-4-[[3-[1-prop-2-ynyl-3-(trifluoromethyl)pyrazol-4-yl]imidazo[1,2-α]pyrazin-8-yl]amino]benzamide;
4-[[3-[1-(cyanomethyl)-3-(trifluoromethyl)pyrazol-4-yl]imidazo[1,2-α]pyrazin-8-yl]amino]-N,2-diethylbenzamide;
N,2-diethyl-4-[[3-[1-(2-fluoroethyl)-3-(trifluoromethyl)pyrazol-4-yl]imidazo[1,2-α]pyrazin-8-yl]amino]benzamide;
[2-chloro-4-[[3-[1-(2,2-difluoroethyl)-3-(trifluoromethyl)pyrazol-4-yl]imidazo[1,2-α]pyrazin-8-yl]amino]phenyl]-[4-[(2S)-pyrrolidine-2-carbonyl]piperazin-1-yl]methanone;
N-[2-(2-aminoethylamino)-2-oxoethyl]-2-chloro-4-[[3-[1-prop-2-ynyl-3-(trifluoromethyl)pyrazol-4-yl]imidazo[1,2-α]pyrazin-8-yl]amino]benzamide;
N-[2-(2-aminoethylamino)-2-oxoethyl]-2-chloro-4-[[3-[1-(2,2-difluoroethyl)-3-(trifluoromethyl)pyrazol-4-yl]imidazo[1,2-α]pyrazin-8-yl]amino]benzamide;
[2-chloro-4-[[3-[1-[(5-methyl-1H-pyrazol-3-yl)methyl]-3-(trifluoromethyl)pyrazol-4-yl]imidazo[1,2-α]pyrazin-8-yl]amino]phenyl]-piperazin-1-ylmethanone;
N-[2-(2-aminoethylamino)-2-oxoethyl]-2-chloro-4-[[3-[1-(cyanomethyl)-3-(trifluoromethyl)pyrazol-4-yl]imidazo[1,2-α]pyrazin-8-yl]amino]benzamide;
[2-chloro-4-[[3-[1-(2,2-difluoroethyl)-3-(trifluoromethyl)pyrazol-4-yl]imidazo[1,2-α]pyrazin-8-yl]amino]phenyl]-piperazin-1-ylmethanone;
N-[2-(2-aminoethoxy)ethyl]-4-[[3-[3-(difluoromethyl)-1-prop-2-enylpyrazol-4-yl]imidazo[1,2-α]pyrazin-8-yl]amino]-2-ethylbenzamide;
N-[2-(2-aminoethoxy)ethyl]-4-[[3-[1-(2,2-difluoroethyl)-3-(difluoromethyl)pyrazol-4-yl]imidazo[1,2-α]pyrazin-8-yl]amino]-2-ethylbenzamide;
N-[2-(2-aminoethoxy)ethyl]-4-[[3-[3-(difluoromethyl)-1-prop-2-ynylpyrazol-4-yl]imidazo[1,2-α]pyrazin-8-yl]amino]-2-ethylbenzamide;
2-chloro-4-[[3-[1-(2,2-difluoroethyl)-3-(trifluoromethyl)pyrazol-4-yl]imidazo[1,2-α]pyrazin-8-yl]amino]-N-methylbenzamide;
2-chloro-N-[2-oxo-2-[[(3S)-pyrrolidin-3-yl]amino]ethyl]-4-[[3-[3-(trifluoromethyl)-1H-pyrazol-4-yl]imidazo[1,2-α]pyrazin-8-yl]amino]benzamide;
N-[(2S)-1-(4-aminopiperidin-1-yl)-1-oxopropan-2-yl]-2-chloro-4-[[3-[3-(trifluoromethyl)-1H-pyrazol-4-yl]imidazo[1,2-α]pyrazin-8-yl]amino]benzamide;
2-chloro-N-[(2S)-1-oxo-1-[[(3S)-pyrrolidin-3-yl]amino]propan-2-yl]-4-[[3-[3-(trifluoromethyl)-1H-pyrazol-4-yl]imidazo[1,2-α]pyrazin-8-yl]amino]benzamide;
tert-butyl N-[(2R)-2-[[2-ethyl-4-[[3-[1-prop-2-ynyl-3-(trifluoromethyl)pyrazol-4-yl]imidazo[1,2-α]pyrazin-8-yl]amino]benzoyl]amino]propyl]carbamate;
tert-butyl N-[(2R)-2-[[2-ethyl-4-[[3-[1-prop-2-enyl-3-(trifluoromethyl)pyrazol-4-yl]imidazo[1,2-α]pyrazin-8-yl]amino]benzoyl]amino]propyl]carbamate;
tert-butyl N-[(2R)-2-[[4-[[3-[1-(2,2-difluoroethyl)-3-(trifluoromethyl)pyrazol-4-yl]imidazo[1,2-α]pyrazin-8-yl]amino]-2-ethylbenzoyl]amino]propyl]carbamate;
2-amino-1-[4-[2-chloro-4-[[3-[3-(trifluoromethyl)-1H-pyrazol-4-yl]imidazo[1,2-α]pyrazin-8-yl]amino]benzoyl]piperazin-1-yl]ethanone;
2-[4-[8-[4-[4-(2-aminoacetyl)piperazine-1-carbonyl]-3-chloroanilino]imidazo[1,2-α]pyrazin-3-yl]-3-(trifluoromethyl)pyrazol-1-yl]acetonitrile;
2-(azetidin-3-yl)-1-[4-[2-chloro-4-[[3-[3-(trifluoromethyl)-1H-pyrazol-4-yl]imidazo[1,2-α]pyrazin-8-yl]amino]benzoyl]piperazin-1-yl]ethanone;
[2-chloro-4-[[3-[1-[(1-methylpyrazol-4-yl)methyl]-3-(trifluoromethyl)pyrazol-4-yl]imidazo[1,2-α]pyrazin-8-yl]amino]phenyl]-piperazin-1-ylmethanone;
[2-chloro-4-[[3-[1-(pyridazin-3-ylmethyl)-3-(trifluoromethyl)pyrazol-4-yl]imidazo[1,2-α]pyrazin-8-yl]amino]phenyl]-piperazin-1-ylmethanone;
N-[2-(4-aminopiperidin-1-yl)-2-oxoethyl]-2-chloro-4-[[3-[3-(trifluoromethyl)-1H-pyrazol-4-yl]imidazo[1,2-α]pyrazin-8-yl]amino]benzamide;
N-[2-[(3R)-3-aminopyrrolidin-1-yl]-2-oxoethyl]-2-chloro-4-[[3-[3-(trifluoromethyl)-1H-pyrazol-4-yl]imidazo[1,2-α]pyrazin-8-yl]amino]benzamide;
N-[2-[(3-aminocyclobutyl)amino]-2-oxoethyl]-2-chloro-4-[[3-[3-(trifluoromethyl)-1H-pyrazol-4-yl]imidazo[1,2-α]pyrazin-8-yl]amino]benzamide;
2-chloro-N-[2-oxo-2-[[(3R)-pyrrolidin-3-yl]amino]ethyl]-4-[[3-[3-(trifluoromethyl)-1H-pyrazol-4-yl]imidazo[1,2-α]pyrazin-8-yl]amino]benzamide;
2-chloro-N-[(2S)-1-oxo-1-piperazin-1-ylpropan-2-yl]-4-[[3-[3-(trifluoromethyl)-1H-pyrazol-4-yl]imidazo[1,2-α]pyrazin-8-yl]amino]benzamide;
N-[2-(3-aminoazetidin-1-yl)-2-oxoethyl]-2-chloro-4-[[3-[3-(trifluoromethyl)-1H-pyrazol-4-yl]imidazo[1,2-α]pyrazin-8-yl]amino]benzamide;
N-[1-[2-chloro-4-[[3-[1-(cyanomethyl)-3-(trifluoromethyl)pyrazol-4-yl]imidazo[1,2-α]pyrazin-8-yl]amino]benzoyl]piperidin-4-yl]piperidine-4-carboxamide;
(2S,4R)—N-[1-[2-chloro-4-[[3-[1-(cyanomethyl)-3-(trifluoromethyl)pyrazol-4-yl]imidazo[1,2-α]pyrazin-8-yl]amino]benzoyl]piperidin-4-yl]-4-hydroxypyrrolidine-2-carboxamide;
1-[1-[2-chloro-4-[[3-[3-(trifluoromethyl)-1H-pyrazol-4-yl]imidazo[1,2-α]pyrazin-8-yl]amino]benzoyl]piperidin-4-yl]-3-[(3S)-pyrrolidin-3-yl]urea;
N-[(2R)-1-aminopropan-2-yl]-2-ethyl-4-[[3-[3-(trifluoromethyl)-1H-pyrazol-4-yl]imidazo[1,2-α]pyrazin-8-yl]amino]benzamide;
N-[2-[(2S)-2-aminopropoxy]ethyl]-2-ethyl-4-[[3-[1-prop-2-enyl-3-(trifluoromethyl)pyrazol-4-yl]imidazo[1,2-α]pyrazin-8-yl]amino]benzamide;
N-[2-[(2S)-2-aminopropoxy]ethyl]-2-ethyl-4-[[3-[1-prop-2-ynyl-3-(trifluoromethyl)pyrazol-4-yl]imidazo[1,2-α]pyrazin-8-yl]amino]benzamide;
N-[2-[(2R)-2-aminopropoxy]ethyl]-2-ethyl-4-[[3-[1-prop-2-enyl-3-(trifluoromethyl)pyrazol-4-yl]imidazo[1,2-α]pyrazin-8-yl]amino]benzamide;
N-[2-[(2R)-2-aminopropoxy]ethyl]-2-ethyl-4-[[3-[1-prop-2-ynyl-3-(trifluoromethyl)pyrazol-4-yl]imidazo[1,2-α]pyrazin-8-yl]amino]benzamide;
N-[2-[(2R)-2-aminopropoxy]ethyl]-2-ethyl-4-[[3-[1-(2-fluoroethyl)-3-(trifluoromethyl)pyrazol-4-yl]imidazo[1,2-α]pyrazin-8-yl]amino]benzamide;
N-[2-[(2R)-2-aminopropoxy]ethyl]-4-[[3-[1-(2,2-difluoroethyl)-3-(trifluoromethyl)pyrazol-4-yl]imidazo[1,2-α]pyrazin-8-yl]amino]-2-ethylbenzamide;
N-[2-[(2S)-2-aminopropoxy]ethyl]-4-[[3-[1-(cyanomethyl)-3-(trifluoromethyl)pyrazol-4-yl]imidazo[1,2-α]pyrazin-8-yl]amino]-2-ethylbenzamide;
N-[2-[(2S)-2-aminopropoxy]ethyl]-4-[[3-[1-(2,2-difluoroethyl)-3-(trifluoromethyl)pyrazol-4-yl]imidazo[1,2-α]pyrazin-8-yl]amino]-2-ethylbenzamide;
(2S,4R)—N-[1-[2-chloro-4-[[3-[3-(trifluoromethyl)-1H-pyrazol-4-yl]imidazo[1,2-α]pyrazin-8-yl]amino]benzoyl]piperidin-4-yl]-4-hydroxypyrrolidine-2-carboxamide;
N-[3-[4-(3-aminopropylamino)butylamino]propyl]-2-ethyl-4-[[3-[1-prop-2-ynyl-3-(trifluoromethyl)pyrazol-4-yl]imidazo[1,2-α]pyrazin-8-yl]amino]benzamide;
N-[2-[(2R)-2-aminopropoxy]ethyl]-4-[[3-[1-(cyanomethyl)-3-(trifluoromethyl)pyrazol-4-yl]imidazo[1,2-α]pyrazin-8-yl]amino]-2-ethylbenzamide;
N-[1-[2-chloro-4-[[3-[3-(trifluoromethyl)-1H-pyrazol-4-yl]imidazo[1,2-α]pyrazin-8-yl]amino]benzoyl]piperidin-4-yl]piperidine-4-carboxamide;
N-[2-[(2S)-2-aminopropoxy]ethyl]-2-ethyl-4-[[3-[1-(2-fluoroethyl)-3-(trifluoromethyl)pyrazol-4-yl]imidazo[1,2-α]pyrazin-8-yl]amino]benzamide;
[2-chloro-4-[[3-[1-[(6-methoxypyridazin-3-yl)methyl]-3-(trifluoromethyl)pyrazol-4-yl]imidazo[1,2-α]pyrazin-8-yl]amino]phenyl]-piperazin-1-ylmethanone;
[2-chloro-4-[[3-[1-[(5-methylpyrimidin-2-yl)methyl]-3-(trifluoromethyl)pyrazol-4-yl]imidazo[1,2-α]pyrazin-8-yl]amino]phenyl]-piperazin-1-ylmethanone;
[2-chloro-4-[[3-[1-[(4-methylpyrimidin-2-yl)methyl]-3-(trifluoromethyl)pyrazol-4-yl]imidazo[1,2-α]pyrazin-8-yl]amino]phenyl]-piperazin-1-ylmethanone;
2-chloro-N-(2-oxo-2-piperazin-1-ylethyl)-4-[[3-[3-(trifluoromethyl)-1H-pyrazol-4-yl]imidazo[1,2-α]pyrazin-8-yl]amino]benzamide;
[2-chloro-4-[[3-[1-[(6-methylpyridazin-3-yl)methyl]-3-(trifluoromethyl)pyrazol-4-yl]imidazo[1,2-α]pyrazin-8-yl]amino]phenyl]-piperazin-1-ylmethanone;
N-[2-[(3S)-3-aminopyrrolidin-1-yl]-2-oxoethyl]-2-chloro-4-[[3-[3-(trifluoromethyl)-1H-pyrazol-4-yl]imidazo[1,2-α]pyrazin-8-yl]amino]benzamide;
2-chloro-N-[2-[2-(dimethylamino)ethylamino]-2-oxoethyl]-4-[[3-[3-(trifluoromethyl)-1H-pyrazol-4-yl]imidazo[1,2-α]pyrazin-8-yl]amino]benzamide;
N-[2-(2-aminoethylamino)-2-oxoethyl]-2-chloro-4-[[3-[3-(trifluoromethyl)-1H-pyrazol-4-yl]imidazo[1,2-α]pyrazin-8-yl]amino]benzamide;
(3-aminoazetidin-1-yl)-[2-chloro-4-[[3-[3-(trifluoromethyl)-1H-pyrazol-4-yl]imidazo[1,2-α]pyrazin-8-yl]amino]phenyl]methanone;
1-[1-[2-chloro-4-[[3-[3-(trifluoromethyl)-1H-pyrazol-4-yl]imidazo[1,2-α]pyrazin-8-yl]amino]benzoyl]azetidin-3-yl]-3-[(3R)-pyrrolidin-3-yl]urea;
[2-chloro-4-[[3-[1-(pyrimidin-2-ylmethyl)-3-(trifluoromethyl)pyrazol-4-yl]imidazo[1,2-α]pyrazin-8-yl]amino]phenyl]-piperazin-1-ylmethanone;
N-[1-[2-chloro-4-[[3-[1-(cyanomethyl)-3-(trifluoromethyl)pyrazol-4-yl]imidazo[1,2-α]pyrazin-8-yl]amino]benzoyl]azetidin-3-yl]piperidine-4-carboxamide;
1-[1-[2-chloro-4-[[3-[1-(cyanomethyl)-3-(trifluoromethyl)pyrazol-4-yl]imidazo[1,2-α]pyrazin-8-yl]amino]benzoyl]azetidin-3-yl]-3-[(3R)-pyrrolidin-3-yl]urea;
1-[1-[2-chloro-4-[[3-[1-(cyanomethyl)-3-(trifluoromethyl)pyrazol-4-yl]imidazo[1,2-α]pyrazin-8-yl]amino]benzoyl]azetidin-3-yl]-3-[(3S)-pyrrolidin-3-yl]urea;
(2S,4R)—N-[1-[2-chloro-4-[[3-[1-(cyanomethyl)-3-(trifluoromethyl)pyrazol-4-yl]imidazo[1,2-α]pyrazin-8-yl]amino]benzoyl]azetidin-3-yl]-4-hydroxypyrrolidine-2-carboxamide;
N-[2-(2-aminoethoxy)ethyl]-2-ethyl-4-[[3-[1-prop-2-ynyl-3-(trifluoromethyl)pyrazol-4-yl]imidazo[1,2-α]pyrazin-8-yl]amino]benzamide;
(2S,4R)—N-[1-[2-chloro-4-[[3-[3-(trifluoromethyl)-1H-pyrazol-4-yl]imidazo[1,2-α]pyrazin-8-yl]amino]benzoyl]azetidin-3-yl]-4-hydroxypyrrolidine-2-carboxamide;
N-[2-(2-amino-2-methylpropoxy)ethyl]-2-ethyl-4-[[3-[3-(trifluoromethyl)-1H-pyrazol-4-yl]imidazo[1,2-α]pyrazin-8-yl]amino]benzamide;
[2-chloro-4-[[3-[1-(1H-pyrazol-3-ylmethyl)-3-(trifluoromethyl)pyrazol-4-yl]imidazo[1,2-α]pyrazin-8-yl]amino]phenyl]-piperazin-1-ylmethanone;
2-[4-[8-[4-[4-(4-aminopiperidine-1-carbonyl)piperazine-1-carbonyl]-3-chloroanilino]imidazo[1,2-α]pyrazin-3-yl]-3-(trifluoromethyl)pyrazol-1-yl]acetonitrile;
2-[4-[8-[4-[4-[(3R)-3-aminopyrrolidine-1-carbonyl]piperazine-1-carbonyl]-3-chloroanilino]imidazo[1,2-α]pyrazin-3-yl]-3-(trifluoromethyl)pyrazol-1-yl]acetonitrile;
2-[4-[8-[4-[4-[(3S)-3-aminopyrrolidine-1-carbonyl]piperazine-1-carbonyl]-3-chloroanilino]imidazo[1,2-α]pyrazin-3-yl]-3-(trifluoromethyl)pyrazol-1-yl]acetonitrile;
[2-chloro-4-[[3-[1-[(2-methylpyrazol-3-yl)methyl]-3-(trifluoromethyl)pyrazol-4-yl]imidazo[1,2-α]pyrazin-8-yl]amino]phenyl]-piperazin-1-ylmethanone;
[2-chloro-4-[[3-[1-(pyrimidin-5-ylmethyl)-3-(trifluoromethyl)pyrazol-4-yl]imidazo[1,2-α]pyrazin-8-yl]amino]phenyl]-piperazin-1-ylmethanone;
[2-chloro-4-[[3-[1-[(1-methylpyrazol-3-yl)methyl]-3-(trifluoromethyl)pyrazol-4-yl]imidazo[1,2-α]pyrazin-8-yl]amino]phenyl]-piperazin-1-ylmethanone;
2-ethyl-N-[3-(prop-2-ynylamino)propyl]-4-[[3-[3-(trifluoromethyl)-1H-pyrazol-4-yl]imidazo[1,2-α]pyrazin-8-yl]amino]benzamide;
[2-chloro-4-[[3-[1-[(1,5-dimethylpyrazol-3-yl)methyl]-3-(trifluoromethyl)pyrazol-4-yl]imidazo[1,2-α]pyrazin-8-yl]amino]phenyl]-piperazin-1-ylmethanone;
2-[4-[8-[3-chloro-4-[4-[(2R)-pyrrolidine-2-carbonyl]piperazine-1-carbonyl]anilino]imidazo[1,2-α]pyrazin-3-yl]-3-(trifluoromethyl)pyrazol-1-yl]acetonitrile;
2-[4-[8-[4-[4-[(2S)-2-aminopropanoyl]piperazine-1-carbonyl]-3-chloroanilino]imidazo[1,2-α]pyrazin-3-yl]-3-(trifluoromethyl)pyrazol-1-yl]acetonitrile;
2-[4-[8-[4-[4-[(2R)-2-aminopropanoyl]piperazine-1-carbonyl]-3-chloroanilino]imidazo[1,2-α]pyrazin-3-yl]-3-(trifluoromethyl)pyrazol-1-yl]acetonitrile;
2-[4-[8-[4-[4-(3-aminocyclobutanecarbonyl)piperazine-1-carbonyl]-3-chloroanilino]imidazo[1,2-α]pyrazin-3-yl]-3-(trifluoromethyl)pyrazol-1-yl]acetonitrile;
2-[4-[8-[3-chloro-4-[4-[(3S)-pyrrolidine-3-carbonyl]piperazine-1-carbonyl]anilino]imidazo[1,2-α]pyrazin-3-yl]-3-(trifluoromethyl)pyrazol-1-yl]acetonitrile;
2-[4-[8-[3-chloro-4-[4-[(3R)-pyrrolidine-3-carbonyl]piperazine-1-carbonyl]anilino]imidazo[1,2-α]pyrazin-3-yl]-3-(trifluoromethyl)pyrazol-1-yl]acetonitrile;
2-[4-[8-[4-[4-[(1S,3R)-3-aminocyclopentanecarbonyl]piperazine-1-carbonyl]-3-chloroanilino]imidazo[1,2-α]pyrazin-3-yl]-3-(trifluoromethyl)pyrazol-1-yl]acetonitrile;
N-[3-(2-aminoethylcarbamoylamino)propyl]-2-ethyl-4-[[3-[3-(trifluoromethyl)-1H-pyrazol-4-yl]imidazo[1,2-α]pyrazin-8-yl]amino]benzamide;
2-[4-[8-[4-[4-[(1R,3S)-3-aminocyclopentanecarbonyl]piperazine-1-carbonyl]-3-chloroanilino]imidazo[1,2-α]pyrazin-3-yl]-3-(trifluoromethyl)pyrazol-1-yl]acetonitrile;
N-[3-[[(2S)-2,6-diaminohexanoyl]amino]propyl]-2-ethyl-4-[[3-[3-(trifluoromethyl)-1H-pyrazol-4-yl]imidazo[1,2-α]pyrazin-8-yl]amino]benzamide;
2-ethyl-N-[3-(prop-2-enylamino)propyl]-4-[[3-[3-(trifluoromethyl)-1H-pyrazol-4-yl]imidazo[1,2-α]pyrazin-8-yl]amino]benzamide;
[2-chloro-4-[[3-[3-(trifluoromethyl)-1H-pyrazol-4-yl]imidazo[1,2-α]pyrazin-8-yl]amino]phenyl]-[4-[(3R)-pyrrolidine-3-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(2R)-pyrrolidine-2-carbonyl]piperazin-1-yl]methanone;
N-(4-aminocyclohexyl)-2-chloro-4-[[3-[1-(cyanomethyl)-3-(trifluoromethyl)pyrazol-4-yl]imidazo[1,2-α]pyrazin-8-yl]amino]benzamide;
[4-[(2S,4R)-4-aminopyrrolidine-2-carbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
N-(2-aminoethoxy)-2-chloro-4-[[3-[1-(cyanomethyl)-3-(trifluoromethyl)pyrazol-4-yl]imidazo[1,2-α]pyrazin-8-yl]amino]benzamide;
2-[4-[8-[4-(4-aminopiperidine-1-carbonyl)-3-chloroanilino]imidazo[1,2-α]pyrazin-3-yl]-3-(trifluoromethyl)pyrazol-1-yl]acetonitrile;
N-[2-[(2S)-2-aminopropoxy]ethyl]-2-ethyl-4-[[3-[3-(trifluoromethyl)-1H-pyrazol-4-yl]imidazo[1,2-α]pyrazin-8-yl]amino]benzamide;
N-[2-[(2R)-2-aminopropoxy]ethyl]-2-ethyl-4-[[3-[3-(trifluoromethyl)-1H-pyrazol-4-yl]imidazo[1,2-α]pyrazin-8-yl]amino]benzamide;
4-[[3-[1-(cyanomethyl)-3-(trifluoromethyl)pyrazol-4-yl]imidazo[1,2-α]pyrazin-8-yl]amino]-N-(3-ethoxypropyl)-2-ethylbenzamide;
(2S)-2-amino-1-[4-[2-chloro-4-[[3-[3-(trifluoromethyl)-1H-pyrazol-4-yl]imidazo[1,2-α]pyrazin-8-yl]amino]benzoyl]piperazin-1-yl]propan-1-one;
(2R)-2-amino-1-[4-[2-chloro-4-[[3-[3-(trifluoromethyl)-1H-pyrazol-4-yl]imidazo[1,2-α]pyrazin-8-yl]amino]benzoyl]piperazin-1-yl]propan-1-one;
[2-chloro-4-[[3-[3-(trifluoromethyl)-1H-pyrazol-4-yl]imidazo[1,2-α]pyrazin-8-yl]amino]phenyl]-[4-[(3S)-pyrrolidine-3-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(2S)-pyrrolidine-2-carbonyl]piperazin-1-yl]methanone;
2-[4-[8-[3-chloro-4-[4-[(2S)-pyrrolidine-2-carbonyl]piperazine-1-carbonyl]anilino]imidazo[1,2-α]pyrazin-3-yl]-3-(trifluoromethyl)pyrazol-1-yl]acetonitrile;
N-[3-[[(2S)-2-amino-3-carbamimidamidopropanoyl]amino]propyl]-2-chloro-4-[[3-[3-(trifluoromethyl)-1H-pyrazol-4-yl]imidazo[1,2-α]pyrazin-8-yl]amino]benzamide;
N-[3-(3-aminopropanoylamino)propyl]-2-ethyl-4-[[3-[3-(trifluoromethyl)-1H-pyrazol-4-yl]imidazo[1,2-α]pyrazin-8-yl]amino]benzamide;
N-[2-[[(2S)-2-amino-3-carbamimidamidopropanoyl]amino]ethyl]-2-ethyl-4-[[3-[3-(trifluoromethyl)-1H-pyrazol-4-yl]imidazo[1,2-α]pyrazin-8-yl]amino]benzamide;
N-(azetidin-3-yl)-4-[2-chloro-4-[[3-[1-(cyanomethyl)-3-(trifluoromethyl)pyrazol-4-yl]imidazo[1,2-α]pyrazin-8-yl]amino]benzoyl]piperazine-1-carboxamide;
N-[3-(4-aminobutanoylamino)propyl]-2-ethyl-4-[[3-[3-(trifluoromethyl)-1H-pyrazol-4-yl]imidazo[1,2-α]pyrazin-8-yl]amino]benzamide;
N-[2-[[(2S)-2-amino-3-carbamimidamidopropanoyl]amino]ethyl]-2-chloro-4-[[3-[3-(trifluoromethyl)-1H-pyrazol-4-yl]imidazo[1,2-α]pyrazin-8-yl]amino]benzamide;
N-[2-(4-aminobutanoylamino)ethyl]-2-ethyl-4-[[3-[3-(trifluoromethyl)-1H-pyrazol-4-yl]imidazo[1,2-α]pyrazin-8-yl]amino]benzamide;
N-[2-(2-aminoethylcarbamoylamino)ethyl]-2-ethyl-4-[[3-[3-(trifluoromethyl)-1H-pyrazol-4-yl]imidazo[1,2-α]pyrazin-8-yl]amino]benzamide;
2-[4-[8-[3-ethyl-4-[4-hydroxy-4-(methylaminomethyl)piperidine-1-carbonyl]anilino]imidazo[1,2-α]pyrazin-3-yl]-3-(trifluoromethyl)pyrazol-1-yl]acetonitrile;
N-[2-(3-aminopropanoylamino)ethyl]-2-ethyl-4-[[3-[3-(trifluoromethyl)-1H-pyrazol-4-yl]imidazo[1,2-α]pyrazin-8-yl]amino]benzamide;
N-[2-[[(2S)-2,6-diaminohexanoyl]amino]ethyl]-2-ethyl-4-[[3-[3-(trifluoromethyl)-1H-pyrazol-4-yl]imidazo[1,2-α]pyrazin-8-yl]amino]benzamide;
N-[2-(2-aminoethylcarbamoylamino)ethyl]-2-chloro-4-[[3-[3-(trifluoromethyl)-1H-pyrazol-4-yl]imidazo[1,2-α]pyrazin-8-yl]amino]benzamide;
N-(3-ethoxypropyl)-2-ethyl-4-[[3-[3-(trifluoromethyl)-1H-pyrazol-4-yl]imidazo[1,2-α]pyrazin-8-yl]amino]benzamide;
2-chloro-N-[2-[[(2S)-2,6-diaminohexanoyl]amino]ethyl]-4-[[3-[3-(trifluoromethyl)-1H-pyrazol-4-yl]imidazo[1,2-α]pyrazin-8-yl]amino]benzamide;
[4-[(1R,2S)-2-aminocyclopropanecarbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
2-[4-[8-[3-chloro-4-(piperazine-1-carbonyl)anilino]imidazo[1,2-α]pyrazin-3-yl]-3-(trifluoromethyl)pyrazol-1-yl]acetonitrile;
N-(6-aminohexyl)-4-[[3-[1-(cyanomethyl)-3-(trifluoromethyl)pyrazol-4-yl]imidazo[1,2-α]pyrazin-8-yl]amino]-2-ethylbenzamide;
N-(2-aminoethoxy)-4-[[3-[1-(cyanomethyl)-3-(trifluoromethyl)pyrazol-4-yl]imidazo[1,2-α]pyrazin-8-yl]amino]-2-ethylbenzamide;
N-(2-aminoethyl)-2-ethyl-4-[[3-[3-(trifluoromethyl)-1H-pyrazol-4-yl]imidazo[1,2-α]pyrazin-8-yl]amino]benzamide;
2-chloro-4-[[3-[1-(cyanomethyl)-3-(trifluoromethyl)pyrazol-4-yl]imidazo[1,2-α]pyrazin-8-yl]amino]-N-ethylbenzamide;
2-chloro-N-(cyanomethyl)-4-[[3-[1-(cyanomethyl)-3-(trifluoromethyl)pyrazol-4-yl]imidazo[1,2-α]pyrazin-8-yl]amino]benzamide;
N-[2-(2-aminoethoxy)ethyl]-4-[[3-[1-but-3-yn-2-yl-3-(trifluoromethyl)pyrazol-4-yl]imidazo[1,2-α]pyrazin-8-yl]amino]-2-ethylbenzamide;
N-[(1S,3R)-3-aminocyclopentyl]-4-[2-chloro-4-[[3-[3-(trifluoromethyl)-1H-pyrazol-4-yl]imidazo[1,2-α]pyrazin-8-yl]amino]benzoyl]piperazine-1-carboxamide;
2-chloro-4-[[3-[1-(cyanomethyl)-3-(trifluoromethyl)pyrazol-4-yl]imidazo[1,2-α]pyrazin-8-yl]amino]-N-(2-hydroxyethyl)benzamide;
N-[2-(2-aminoethoxy)ethyl]-2-ethyl-4-[[3-[1-[(5-methyl-1,2-oxazol-3-yl)methyl]-3-(trifluoromethyl)pyrazol-4-yl]imidazo[1,2-α]pyrazin-8-yl]amino]benzamide;
2-[4-[8-[3-chloro-4-(3-hydroxyazetidine-1-carbonyl)anilino]imidazo[1,2-α]pyrazin-3-yl]-3-(trifluoromethyl)pyrazol-1-yl]acetonitrile;
2-chloro-4-[[3-[1-(cyanomethyl)-3-(trifluoromethyl)pyrazol-4-yl]imidazo[1,2-α]pyrazin-8-yl]amino]-N-[(2R)-2-hydroxypropyl]benzamide;
N-[(1R,3S)-3-aminocyclopentyl]-4-[2-chloro-4-[[3-[3-(trifluoromethyl)-1H-pyrazol-4-yl]imidazo[1,2-α]pyrazin-8-yl]amino]benzoyl]piperazine-1-carboxamide;
2-chloro-4-[[3-[1-(cyanomethyl)-3-(trifluoromethyl)pyrazol-4-yl]imidazo[1,2-α]pyrazin-8-yl]amino]-N-(2-methoxyethyl)benzamide;
2-chloro-4-[[3-[1-(cyanomethyl)-3-(trifluoromethyl)pyrazol-4-yl]imidazo[1,2-α]pyrazin-8-yl]amino]-N-[(2S)-2-hydroxypropyl]benzamide;
N-[2-(2-aminoethoxy)ethyl]-2-ethyl-4-[[3-[1-(oxetan-3-yl)-3-(trifluoromethyl)pyrazol-4-yl]imidazo[1,2-α]pyrazin-8-yl]amino]benzamide;
2-[4-[8-[3-chloro-4-(3-hydroxy-3-methylazetidine-1-carbonyl)anilino]imidazo[1,2-α]pyrazin-3-yl]-3-(trifluoromethyl)pyrazol-1-yl]acetonitrile;
2-[4-[8-[3-chloro-4-[(3S)-3-hydroxypyrrolidine-1-carbonyl]anilino]imidazo[1,2-α]pyrazin-3-yl]-3-(trifluoromethyl)pyrazol-1-yl]acetonitrile;
2-[4-[8-[3-chloro-4-[(3R)-3-hydroxypyrrolidine-1-carbonyl]anilino]imidazo[1,2-α]pyrazin-3-yl]-3-(trifluoromethyl)pyrazol-1-yl]acetonitrile;
[4-[(2R,4S)-4-aminopyrrolidine-2-carbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
N-(azetidin-3-yl)-4-[2-chloro-4-[[3-[1-(2-cyanoethyl)-3-(trifluoromethyl)pyrazol-4-yl]imidazo[1,2-α]pyrazin-8-yl]amino]benzoyl]piperazine-1-carboxamide;
[2-chloro-4-[[3-[3-(trifluoromethyl)-1H-pyrazol-4-yl]imidazo[1,2-α]pyrazin-8-yl]amino]phenyl]-[4-[(3S)-3-hydroxypiperidine-3-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(3R)-3-hydroxypiperidine-3-carbonyl]piperazin-1-yl]methanone;
N-[2-(2-aminoethoxy)ethyl]-2-chloro-4-[[3-[1-prop-2-ynyl-3-(trifluoromethyl)pyrazol-4-yl]imidazo[1,2-α]pyrazin-8-yl]amino]benzamide;
N-[2-(2-aminoethoxy)ethyl]-2-chloro-4-[[3-[1-(2-fluoroethyl)-3-(trifluoromethyl)pyrazol-4-yl]imidazo[1,2-α]pyrazin-8-yl]amino]benzamide;
N-(cyanomethyl)-4-[[3-[1-(cyanomethyl)-3-(trifluoromethyl)pyrazol-4-yl]imidazo[1,2-α]pyrazin-8-yl]amino]-2-ethylbenzamide;
N-[2-(2-aminoethoxy)ethyl]-2-chloro-4-[[3-[1-(2,2-difluoroethyl)-3-(trifluoromethyl)pyrazol-4-yl]imidazo[1,2-α]pyrazin-8-yl]amino]benzamide;
N-[2-(2-aminoethoxy)ethyl]-4-[[3-[1-(2-fluoroethyl)-3-(trifluoromethyl)pyrazol-4-yl]imidazo[1,2-α]pyrazin-8-yl]amino]-2-methylbenzamide;
N-[2-(2-aminoethoxy)ethyl]-4-[[3-[1-(2,2-difluoroethyl)-3-(trifluoromethyl)pyrazol-4-yl]imidazo[1,2-α]pyrazin-8-yl]amino]-2-methylbenzamide;
N-(4-aminocyclohexyl)-4-[[3-[1-(cyanomethyl)-3-(trifluoromethyl)pyrazol-4-yl]imidazo[1,2-α]pyrazin-8-yl]amino]-2-ethylbenzamide;
3-[4-[8-[4-[4-(3-aminopropanoyl)piperazine-1-carbonyl]-3-chloroanilino]imidazo[1,2-α]pyrazin-3-yl]-3-(trifluoromethyl)pyrazol-1-yl]propanenitrile;
N-[2-(2-aminoethoxy)ethyl]-2-chloro-4-[[3-[1-(cyanomethyl)-3-(trifluoromethyl)pyrazol-4-yl]imidazo[1,2-α]pyrazin-8-yl]amino]benzamide;
N-(2-aminoethyl)-4-[2-chloro-4-[[3-[1-(2-cyanoethyl)-3-(trifluoromethyl)pyrazol-4-yl]imidazo[1,2-α]pyrazin-8-yl]amino]benzoyl]piperazine-1-carboxamide;
4-[2-chloro-4-[[3-[1-(2-cyanoethyl)-3-(trifluoromethyl)pyrazol-4-yl]imidazo[1,2-α]pyrazin-8-yl]amino]benzoyl]-N-[(3S)-pyrrolidin-3-yl]piperazine-1-carboxamide;
2-[4-[8-[3-ethyl-4-(piperazine-1-carbonyl)anilino]imidazo[1,2-α]pyrazin-3-yl]-3-(trifluoromethyl)pyrazol-1-yl]acetonitrile;
4-[2-chloro-4-[[3-[1-(2-cyanoethyl)-3-(trifluoromethyl)pyrazol-4-yl]imidazo[1,2-α]pyrazin-8-yl]amino]benzoyl]-N-[(3R)-pyrrolidin-3-yl]piperazine-1-carboxamide;
N-[2-(2-aminoethoxy)ethyl]-4-[[3-[1-(cyanomethyl)-3-(trifluoromethyl)pyrazol-4-yl]imidazo[1,2-α]pyrazin-8-yl]amino]-2-methylbenzamide;
N-[2-(1-aminocyclopropyl)ethyl]-2-ethyl-4-[[3-[3-(trifluoromethyl)-1H-pyrazol-4-yl]imidazo[1,2-α]pyrazin-8-yl]amino]benzamide;
N-(2-aminoethyl)-4-[2-chloro-4-[[3-[1-(cyanomethyl)-3-(trifluoromethyl)pyrazol-4-yl]imidazo[1,2-α]pyrazin-8-yl]amino]benzoyl]piperazine-1-carboxamide;
N-(3-aminocyclobutyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
[4-(3-aminoazetidine-1-carbonyl)piperazin-1-yl]-[2-chloro-4-[[3-[3-(trifluoromethyl)-1H-pyrazol-4-yl]imidazo[1,2-α]pyrazin-8-yl]amino]phenyl]methanone;
N-[2-(2-aminoethoxy)ethyl]-2-ethyl-4-[[3-[1-(3-hydroxy-2-methylpropyl)-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(2-hydroxybutyl)-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(2-pyrrolidin-1-ylethyl)-3-(trifluoromethyl)pyrazol-4-yl]imidazo[1,2-α]pyrazin-8-yl]amino]benzamide;
N-[3-[4-(3-aminopropylamino)butylamino]propyl]-2-ethyl-4-[[3-[3-(trifluoromethyl)-1H-pyrazol-4-yl]imidazo[1,2-α]pyrazin-8-yl]amino]benzamide;
2-[4-[8-[4-[2-(2-aminoethoxy)ethylcarbamoyl]-3-ethylanilino]imidazo[1,2-α]pyrazin-3-yl]-3-(trifluoromethyl)pyrazol-1-yl]acetic acid;
N-[2-(2-aminoethoxy)ethyl]-2-ethyl-4-[[3-[1-prop-2-enyl-3-(trifluoromethyl)pyrazol-4-yl]imidazo[1,2-α]pyrazin-8-yl]amino]benzamide;
N-[2-(2-aminoethoxy)ethyl]-4-[[3-[1-cyclobutyl-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(2-chloroprop-2-enyl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(3-chlorobut-3-en-2-yl)-3-(trifluoromethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-(2,2,2-trifluoroethyl)-3-(trifluoromethyl)pyrazol-4-yl]imidazo[1,2-α]pyrazin-8-yl]amino]benzamide;
N-[2-(2-aminoethoxy)ethyl]-4-[[3-[1-[(2,2-difluorocyclopropyl)methyl]-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(1-chloro-3-hydroxypropan-2-yl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-[2-(chloromethyl)-3-hydroxy-2-methylpropyl]-3-(trifluoromethyl)pyrazol-4-yl]imidazo[1,2-α]pyrazin-8-yl]amino]-2-ethylbenzamide;
N,2-diethyl-4-[[3-[3-(trifluoromethyl)-1H-pyrazol-4-yl]imidazo[1,2-α]pyrazin-8-yl]amino]benzamide;
2-chloro-N-methyl-4-[[3-[1-(pyrimidin-4-ylmethyl)-3-(trifluoromethyl)pyrazol-4-yl]imidazo[1,2-α]pyrazin-8-yl]amino]benzamide;
2-ethyl-N-methyl-4-[[3-[1-(pyrimidin-4-ylmethyl)-3-(trifluoromethyl)pyrazol-4-yl]imidazo[1,2-α]pyrazin-8-yl]amino]benzamide;
4-[[3-[1-(2-aminoethyl)-3-(trifluoromethyl)pyrazol-4-yl]imidazo[1,2-α]pyrazin-8-yl]amino]-2-ethyl-N-methylbenzamide;
2-chloro-N-methyl-4-[[3-[1-(1H-pyrazol-3-ylmethyl)-3-(trifluoromethyl)pyrazol-4-yl]imidazo[1,2-α]pyrazin-8-yl]amino]benzamide;
[4-(4-aminopiperidine-1-carbonyl)piperazin-1-yl]-[2-chloro-4-[[3-[3-(trifluoromethyl)-1H-pyrazol-4-yl]imidazo[1,2-α]pyrazin-8-yl]amino]phenyl]methanone;
2-[4-[8-[4-[4-(3-aminopropanoyl)piperazine-1-carbonyl]-3-chloroanilino]imidazo[1,2-α]pyrazin-3-yl]-3-(trifluoromethyl)pyrazol-1-yl]acetonitrile;
N-(4-aminocyclohexyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
4-[2-chloro-4-[[3-[1-(cyanomethyl)-3-(trifluoromethyl)pyrazol-4-yl]imidazo[1,2-α]pyrazin-8-yl]amino]benzoyl]-N-[(3R)-pyrrolidin-3-yl]piperazine-1-carboxamide;
N-(3-aminocyclobutyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
N-(2-aminoethyl)-4-[2-chloro-4-[[3-[1-(2-methoxyethyl)-3-(trifluoromethyl)pyrazol-4-yl]imidazo[1,2-α]pyrazin-8-yl]amino]benzoyl]piperazine-1-carboxamide;
N-(4-aminocyclohexyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
N-[(1S,3S)-3-aminocyclopentyl]-4-[2-chloro-4-[[3-[3-(trifluoromethyl)-1H-pyrazol-4-yl]imidazo[1,2-α]pyrazin-8-yl]amino]benzoyl]piperazine-1-carboxamide;
4-[2-chloro-4-[[3-[1-(2-methoxyethyl)-3-(trifluoromethyl)pyrazol-4-yl]imidazo[1,2-α]pyrazin-8-yl]amino]benzoyl]-N-[(3S)-pyrrolidin-3-yl]piperazine-1-carboxamide;
3-amino-1-[4-[2-chloro-4-[[3-[1-(2-methoxyethyl)-3-(trifluoromethyl)pyrazol-4-yl]imidazo[1,2-α]pyrazin-8-yl]amino]benzoyl]piperazin-1-yl]propan-1-one;
[4-[(2S,4S)-4-aminopyrrolidine-2-carbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
4-[2-chloro-4-[[3-[1-(2-methoxyethyl)-3-(trifluoromethyl)pyrazol-4-yl]imidazo[1,2-α]pyrazin-8-yl]amino]benzoyl]-N-[(3R)-pyrrolidin-3-yl]piperazine-1-carboxamide;
4-[2-chloro-4-[[3-[1-(cyanomethyl)-3-(trifluoromethyl)pyrazol-4-yl]imidazo[1,2-α]pyrazin-8-yl]amino]benzoyl]-N-[(3S)-pyrrolidin-3-yl]piperazine-1-carboxamide;
2-ethyl-N-methyl-4-[[3-[1-[2-(methylamino)ethyl]-3-(trifluoromethyl)pyrazol-4-yl]imidazo[1,2-α]pyrazin-8-yl]amino]benzamide;
N-[2-(3-aminopropanoylamino)ethyl]-2-chloro-4-[[3-[3-(trifluoromethyl)-1H-pyrazol-4-yl]imidazo[1,2-α]pyrazin-8-yl]amino]benzamide;
N-[2-(4-aminobutanoylamino)ethyl]-2-chloro-4-[[3-[3-(trifluoromethyl)-1H-pyrazol-4-yl]imidazo[1,2-α]pyrazin-8-yl]amino]benzamide;
N-[2-(2-aminoethoxy)ethyl]-2-ethyl-4-[[3-[1-(2-fluoroethyl)-3-(trifluoromethyl)pyrazol-4-yl]imidazo[1,2-α]pyrazin-8-yl]amino]benzamide;
N-[2-(2-aminoethoxy)ethyl]-4-[[3-[1-(2,2-difluoroethyl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(cyclobutylmethyl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-[(3,3-difluorocyclobutyl)methyl]-3-(trifluoromethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-(2-hydroxyethyl)-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(3-hydroxy-2,2-dimethylpropyl)-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(oxolan-3-ylmethyl)-3-(trifluoromethyl)pyrazol-4-yl]imidazo[1,2-α]pyrazin-8-yl]amino]benzamide;
2-chloro-N-[(3R)-1-[(2S,4R)-4-hydroxypyrrolidine-2-carbonyl]pyrrolidin-3-yl]-4-[[3-[3-(trifluoromethyl)-1H-pyrazol-4-yl]imidazo[1,2-α]pyrazin-8-yl]amino]benzamide;
N-[2-(2-aminoethoxy)ethyl]-4-[[3-[1-[2-(dimethylamino)ethyl]-3-(trifluoromethyl)pyrazol-4-yl]imidazo[1,2-α]pyrazin-8-yl]amino]-2-ethylbenzamide;
ethyl 2-[4-[8-[4-[2-(2-aminoethoxy)ethylcarbamoyl]-3-ethylanilino]imidazo[1,2-α]pyrazin-3-yl]-3-(trifluoromethyl)pyrazol-1-yl]acetate;
N-[2-(2-aminoethoxy)ethyl]-2-ethyl-4-[[3-[1-(3-hydroxy-3-methylbutyl)-3-(trifluoromethyl)pyrazol-4-yl]imidazo[1,2-α]pyrazin-8-yl]amino]benzamide;
N-[2-(2-aminoethoxy)ethyl]-2-ethyl-4-[[3-[1-(3-hydroxypropyl)-3-(trifluoromethyl)pyrazol-4-yl]imidazo[1,2-α]pyrazin-8-yl]amino]benzamide;
2-chloro-N-[(3S)-1-[(2S,4R)-4-hydroxypyrrolidine-2-carbonyl]pyrrolidin-3-yl]-4-[[3-[3-(trifluoromethyl)-1H-pyrazol-4-yl]imidazo[1,2-α]pyrazin-8-yl]amino]benzamide;
N-[2-(2-aminoethoxy)ethyl]-4-[[3-[1-(2,3-dihydroxypropyl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(2,4-dihydroxybutyl)-3-(trifluoromethyl)pyrazol-4-yl]imidazo[1,2-α]pyrazin-8-yl]amino]-2-ethylbenzamide;
N-[2-(2-aminoethoxy)ethyl]-4-[[3-[1-(2-amino-2-oxoethyl)-3-(trifluoromethyl)pyrazol-4-yl]imidazo[1,2-α]pyrazin-8-yl]amino]-2-ethylbenzamide;
N-[2-(2-aminoethoxy)ethyl]-2-ethyl-4-[[3-[1-(2-morpholin-4-ylethyl)-3-(trifluoromethyl)pyrazol-4-yl]imidazo[1,2-α]pyrazin-8-yl]amino]benzamide;
(4-aminopiperidin-1-yl)-[2-chloro-4-[[3-[3-(trifluoromethyl)-1H-pyrazol-4-yl]imidazo[1,2-α]pyrazin-8-yl]amino]phenyl]methanone;
N-(5-aminopentyl)-2-ethyl-4-[[3-[3-(trifluoromethyl)-1H-pyrazol-4-yl]imidazo[1,2-α]pyrazin-8-yl]amino]benzamide;
[(3S)-3-aminopyrrolidin-1-yl]-[2-chloro-4-[[3-[3-(trifluoromethyl)-1H-pyrazol-4-yl]imidazo[1,2-α]pyrazin-8-yl]amino]phenyl]methanone;
[2-chloro-4-[[3-[3-(trifluoromethyl)-1H-pyrazol-4-yl]imidazo[1,2-α]pyrazin-8-yl]amino]phenyl]-[(3S)-3-methylpiperazin-1-yl]methanone;
N-(2-aminoethyl)-2-chloro-N-methyl-4-[[3-[3-(trifluoromethyl)-1H-pyrazol-4-yl]imidazo[1,2-α]pyrazin-8-yl]amino]benzamide;
[2-chloro-4-[[3-[3-(trifluoromethyl)-1H-pyrazol-4-yl]imidazo[1,2-α]pyrazin-8-yl]amino]phenyl]-[(3R)-3-methylpiperazin-1-yl]methanone;
[(3R)-3-aminopyrrolidin-1-yl]-[2-chloro-4-[[3-[3-(trifluoromethyl)-1H-pyrazol-4-yl]imidazo[1,2-α]pyrazin-8-yl]amino]phenyl]methanone;
[2-chloro-4-[[3-[3-(trifluoromethyl)-1H-pyrazol-4-yl]imidazo[1,2-α]pyrazin-8-yl]amino]phenyl]-[(2S)-2-methylpiperazin-1-yl]methanone;
[4-[(3R)-3-aminopyrrolidine-1-carbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
[4-(3-aminocyclobutanecarbonyl)piperazin-1-yl]-[2-chloro-4-[[3-[3-(trifluoromethyl)-1H-pyrazol-4-yl]imidazo[1,2-α]pyrazin-8-yl]amino]phenyl]methanone;
[4-[(3S)-3-aminopyrrolidine-1-carbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
N-(5-aminopentyl)-4-[[3-[1-(cyanomethyl)-3-(trifluoromethyl)pyrazol-4-yl]imidazo[1,2-α]pyrazin-8-yl]amino]-2-ethylbenzamide;
[4-(3-aminocyclobutanecarbonyl)piperazin-1-yl]-[2-chloro-4-[[3-[3-(trifluoromethyl)-1H-pyrazol-4-yl]imidazo[1,2-α]pyrazin-8-yl]amino]phenyl]methanone;
(2S)-2-amino-1-[4-[2-methyl-4-[[3-[3-(trifluoromethyl)-1H-pyrazol-4-yl]imidazo[1,2-α]pyrazin-8-yl]amino]benzoyl]piperazin-1-yl]propan-1-one;
[2-methyl-4-[[3-[3-(trifluoromethyl)-1H-pyrazol-4-yl]imidazo[1,2-α]pyrazin-8-yl]amino]phenyl]-[4-[(3R)-pyrrolidine-3-carbonyl]piperazin-1-yl]methanone;
[2-methyl-4-[[3-[3-(trifluoromethyl)-1H-pyrazol-4-yl]imidazo[1,2-α]pyrazin-8-yl]amino]phenyl]-[4-[(3S)-pyrrolidine-3-carbonyl]piperazin-1-yl]methanone;
(2R)-2-amino-1-[4-[2-methyl-4-[[3-[3-(trifluoromethyl)-1H-pyrazol-4-yl]imidazo[1,2-α]pyrazin-8-yl]amino]benzoyl]piperazin-1-yl]propan-1-one;
2-ethyl-N-methyl-4-[[3-[1-prop-2-ynyl-3-(trifluoromethyl)pyrazol-4-yl]imidazo[1,2-α]pyrazin-8-yl]amino]benzamide;
N-(6-aminohexyl)-2-ethyl-4-[[3-[3-(trifluoromethyl)-1H-pyrazol-4-yl]imidazo[1,2-α]pyrazin-8-yl]amino]benzamide;
4-[[3-[1-(2-cyanoethyl)-3-(trifluoromethyl)pyrazol-4-yl]imidazo[1,2-α]pyrazin-8-yl]amino]-2-ethyl-N-methylbenzamide;
N-[3-(3-aminopropanoylamino)propyl]-2-chloro-4-[[3-[3-(trifluoromethyl)-1H-pyrazol-4-yl]imidazo[1,2-α]pyrazin-8-yl]amino]benzamide;
4-[[3-[1-(2-amino-2-oxoethyl)-3-(trifluoromethyl)pyrazol-4-yl]imidazo[1,2-α]pyrazin-8-yl]amino]-2-ethyl-N-methylbenzamide;
[4-[(1R,3S)-3-aminocyclopentanecarbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
4-[[3-[1-(cyanomethyl)-3-(trifluoromethyl)pyrazol-4-yl]imidazo[1,2-α]pyrazin-8-yl]amino]-2-ethyl-N-methylbenzamide;
[4-[(1S,3R)-3-aminocyclopentanecarbonyl]piperazin-1-yl]-[2-chloro-4-[[3-[3-(trifluoromethyl)-1H-pyrazol-4-yl]imidazo[1,2-α]pyrazin-8-yl]amino]phenyl]methanone;
4-[[3-[1-(3-cyanopropyl)-3-(trifluoromethyl)pyrazol-4-yl]imidazo[1,2-α]pyrazin-8-yl]amino]-2-ethyl-N-methylbenzamide;
N-[3-(4-aminobutanoylamino)propyl]-2-chloro-4-[[3-[3-(trifluoromethyl)-1H-pyrazol-4-yl]imidazo[1,2-α]pyrazin-8-yl]amino]benzamide;
2-chloro-N-[3-[[(2S)-2,6-diaminohexanoyl]amino]propyl]-4-[[3-[3-(trifluoromethyl)-1H-pyrazol-4-yl]imidazo[1,2-α]pyrazin-8-yl]amino]benzamide;
N-[2-(2-aminoethoxy)ethyl]-4-[[3-[3-(difluoromethyl)-1-propylpyrazol-4-yl]imidazo[1,2-α]pyrazin-8-yl]amino]-2-ethylbenzamide;
N-[2-(2-aminoethoxy)ethyl]-4-[[3-[3-(difluoromethyl)-1-methylpyrazol-4-yl]imidazo[1,2-α]pyrazin-8-yl]amino]-2-ethylbenzamide;
4-[[3-[1-(azetidin-3-yl)-3-(trifluoromethyl)pyrazol-4-yl]imidazo[1,2-α]pyrazin-8-yl]amino]-2-chloro-N-methylbenzamide;
N-[(2S)-1-aminopropan-2-yl]-2-ethyl-4-[[3-[3-(trifluoromethyl)-1H-pyrazol-4-yl]imidazo[1,2-α]pyrazin-8-yl]amino]benzamide;
2-ethyl-N-methyl-4-[[3-[3-(trifluoromethyl)-1H-pyrazol-4-yl]imidazo[1,2-α]pyrazin-8-yl]amino]benzamide;
2-chloro-N-methyl-4-[[3-[1-prop-2-ynyl-3-(trifluoromethyl)pyrazol-4-yl]imidazo[1,2-α]pyrazin-8-yl]amino]benzamide;
[2-chloro-4-[[3-[3-(trifluoromethyl)-1H-pyrazol-4-yl]imidazo[1,2-α]pyrazin-8-yl]amino]phenyl]-[4-(3-hydroxypiperidine-3-carbonyl)piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[rac-(3R,5R)-5-hydroxypiperidine-3-carbonyl]piperazin-1-yl]methanone;
[2-ethyl-4-[[3-[3-(trifluoromethyl)-1H-pyrazol-4-yl]imidazo[1,2-α]pyrazin-8-yl]amino]phenyl]-[4-(piperidine-4-carbonyl)piperazin-1-yl]methanone;
[2-ethyl-4-[[3-[3-(trifluoromethyl)-1H-pyrazol-4-yl]imidazo[1,2-α]pyrazin-8-yl]amino]phenyl]-[4-(4-hydroxypiperidine-4-carbonyl)piperazin-1-yl]methanone;
2-chloro-4-[[3-[1-(cyanomethyl)-3-(trifluoromethyl)pyrazol-4-yl]imidazo[1,2-α]pyrazin-8-yl]amino]-N-methylbenzamide;
4-[[3-[1-(2-amino-2-oxoethyl)-3-(trifluoromethyl)pyrazol-4-yl]imidazo[1,2-α]pyrazin-8-yl]amino]-2-chloro-N-methylbenzamide;
2-chloro-4-[[3-[1-(2-cyanoethyl)-3-(trifluoromethyl)pyrazol-4-yl]imidazo[1,2-α]pyrazin-8-yl]amino]-N-methylbenzamide;
2-chloro-4-[[3-[1-(3-cyanopropyl)-3-(trifluoromethyl)pyrazol-4-yl]imidazo[1,2-α]pyrazin-8-yl]amino]-N-methylbenzamide;
N-[2-(2-aminoethoxy)ethyl]-2-methyl-4-[[3-[3-(trifluoromethyl)-1H-pyrazol-4-yl]imidazo[1,2-α]pyrazin-8-yl]amino]benzamide;
N-[2-(2-aminoethoxy)ethyl]-2-chloro-4-[[3-[3-(trifluoromethyl)-1H-pyrazol-4-yl]imidazo[1,2-α]pyrazin-8-yl]amino]benzamide;
N-(4-aminobutyl)-2-chloro-4-[[3-[3-(trifluoromethyl)-1H-pyrazol-4-yl]imidazo[1,2-α]pyrazin-8-yl]amino]benzamide;
N-(2-aminoethyl)-2-chloro-4-[[3-[3-(trifluoromethyl)-1H-pyrazol-4-yl]imidazo[1,2-α]pyrazin-8-yl]amino]benzamide;
N-(3-aminopropyl)-2-chloro-4-[[3-[3-(trifluoromethyl)-1H-pyrazol-4-yl]imidazo[1,2-α]pyrazin-8-yl]amino]benzamide;
N-(3-aminopropyl)-2-ethyl-4-[[3-[3-(trifluoromethyl)-1H-pyrazol-4-yl]imidazo[1,2-α]pyrazin-8-yl]amino]benzamide;
2-[4-[8-[3-chloro-4-[4-(piperidine-4-carbonyl)piperazine-1-carbonyl]anilino]imidazo[1,2-α]pyrazin-3-yl]-3-(trifluoromethyl)pyrazol-1-yl]acetonitrile;
(2S,4R)—N-[3-[[4-[[3-[1-(cyanomethyl)-3-(trifluoromethyl)pyrazol-4-yl]imidazo[1,2-α]pyrazin-8-yl]amino]-2-methylbenzoyl]amino]propyl]-4-hydroxypyrrolidine-2-carboxamide;
(2S,4R)—N-[3-[[2-chloro-4-[[3-[3-(trifluoromethyl)-1H-pyrazol-4-yl]imidazo[1,2-α]pyrazin-8-yl]amino]benzoyl]amino]cyclobutyl]-4-hydroxypyrrolidine-2-carboxamide;
2-chloro-N-[1-[(2S,4R)-4-hydroxypyrrolidine-2-carbonyl]azetidin-3-yl]-4-[[3-[3-(trifluoromethyl)-1H-pyrazol-4-yl]imidazo[1,2-α]pyrazin-8-yl]amino]benzamide;
[4-(aminomethyl)piperidin-1-yl]-[2-ethyl-4-[[3-[3-(trifluoromethyl)-1H-pyrazol-4-yl]imidazo[1,2-α]pyrazin-8-yl]amino]phenyl]methanone;
rac-(2R,4S)—N-[3-[[2-chloro-4-[[3-[3-(trifluoromethyl)-1H-pyrazol-4-yl]imidazo[1,2-α]pyrazin-8-yl]amino]benzoyl]amino]cyclopentyl]-4-hydroxypyrrolidine-2-carboxamide;
(2S,4R)—N-[4-[[2-chloro-4-[[3-[3-(trifluoromethyl)-1H-pyrazol-4-yl]imidazo[1,2-α]pyrazin-8-yl]amino]benzoyl]amino]cyclohexyl]-4-hydroxypyrrolidine-2-carboxamide;
(2S,4R)—N-[2-[[4-[[3-[1-(cyanomethyl)-3-(trifluoromethyl)pyrazol-4-yl]imidazo[1,2-α]pyrazin-8-yl]amino]-2-methylbenzoyl]amino]ethyl]-4-hydroxypyrrolidine-2-carboxamide;
4-[4-[[3-[1-(cyanomethyl)-3-(trifluoromethyl)pyrazol-4-yl]imidazo[1,2-α]pyrazin-8-yl]amino]-2-methylbenzoyl]-N-[(3S,4S)-4-hydroxypyrrolidin-3-yl]piperazine-1-carboxamide;
2-chloro-N-[(3S)-pyrrolidin-3-yl]-4-[[3-[3-(trifluoromethyl)-1H-pyrazol-4-yl]imidazo[1,2-α]pyrazin-8-yl]amino]benzamide;
2-chloro-N-[(3R)-pyrrolidin-3-yl]-4-[[3-[3-(trifluoromethyl)-1H-pyrazol-4-yl]imidazo[1,2-α]pyrazin-8-yl]amino]benzamide;
2-[4-[8-[4-[4-[(3S,4R)-3-hydroxypiperidine-4-carbonyl]piperazine-1-carbonyl]-3-methylanilino]imidazo[1,2-α]pyrazin-3-yl]-3-(trifluoromethyl)pyrazol-1-yl]acetonitrile;
2-chloro-N-[1-[(2S,4R)-4-hydroxypyrrolidine-2-carbonyl]piperidin-4-yl]-4-[[3-[3-(trifluoromethyl)-1H-pyrazol-4-yl]imidazo[1,2-α]pyrazin-8-yl]amino]benzamide;
2-[4-[8-[4-[4-[(3R,4S)-3-hydroxypiperidine-4-carbonyl]piperazine-1-carbonyl]-3-methylanilino]imidazo[1,2-α]pyrazin-3-yl]-3-(trifluoromethyl)pyrazol-1-yl]acetonitrile;
N-(3-aminocyclobutyl)-2-chloro-4-[[3-[3-(trifluoromethyl)-1H-pyrazol-4-yl]imidazo[1,2-α]pyrazin-8-yl]amino]benzamide;
N-(4-aminocyclohexyl)-2-chloro-4-[[3-[3-(trifluoromethyl)-1H-pyrazol-4-yl]imidazo[1,2-α]pyrazin-8-yl]amino]benzamide;
2-chloro-N-[1-[rac-(2R,4S)-4-hydroxypyrrolidine-2-carbonyl]piperidin-3-yl]-4-[[3-[3-(trifluoromethyl)-1H-pyrazol-4-yl]imidazo[1,2-α]pyrazin-8-yl]amino]benzamide;
2-chloro-N-methyl-4-[[3-[3-(trifluoromethyl)-1H-pyrazol-4-yl]imidazo[1,2-α]pyrazin-8-yl]amino]benzamide;
4-[2-chloro-4-[[3-[3-(trifluoromethyl)-1H-pyrazol-4-yl]imidazo[1,2-α]pyrazin-8-yl]amino]benzoyl]-N-[(3S)-pyrrolidin-3-yl]piperazine-1-carboxamide;
N-(3-aminopropyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
4-[2-chloro-4-[[3-[3-(trifluoromethyl)-1H-pyrazol-4-yl]imidazo[1,2-α]pyrazin-8-yl]amino]benzoyl]-N-[(3R)-pyrrolidin-3-yl]piperazine-1-carboxamide;
N-(2-aminoethyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
N-(azetidin-3-ylmethyl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
N-(azetidin-3-yl)-4-[2-chloro-4-[[3-[3-(trifluoromethyl)-1H-pyrazol-4-yl]imidazo[1,2-α]pyrazin-8-yl]amino]benzoyl]piperazine-1-carboxamide;
4-amino-1-[4-[2-chloro-4-[[3-[3-(trifluoromethyl)-1H-pyrazol-4-yl]imidazo[1,2-α]pyrazin-8-yl]amino]benzoyl]piperazin-1-yl]butan-1-one;
3-amino-1-[4-[2-chloro-4-[[3-[3-(trifluoromethyl)-1H-pyrazol-4-yl]imidazo[1,2-α]pyrazin-8-yl]amino]benzoyl]piperazin-1-yl]propan-1-one;
[2-chloro-4-[[3-[3-(trifluoromethyl)-1H-pyrazol-4-yl]imidazo[1,2-α]pyrazin-8-yl]amino]phenyl]-[4-(piperidine-4-carbonyl)piperazin-1-yl]methanone;
2-chloro-N,N-dimethyl-4-[[3-[3-(trifluoromethyl)-1H-pyrazol-4-yl]imidazo[1,2-α]pyrazin-8-yl]amino]benzamide;
N-(3-aminocyclobutyl)-2-chloro-4-[[3-[3-(trifluoromethyl)-1H-pyrazol-4-yl]imidazo[1,2-α]pyrazin-8-yl]amino]benzamide;
2-chloro-4-[[3-[3-(trifluoromethyl)-1H-pyrazol-4-yl]imidazo[1,2-α]pyrazin-8-yl]amino]benzamide;
N-(4-aminocyclohexyl)-2-chloro-4-[[3-[3-(trifluoromethyl)-1H-pyrazol-4-yl]imidazo[1,2-α]pyrazin-8-yl]amino]benzamide;
N-(azetidin-3-yl)-2-chloro-4-[[3-[3-(trifluoromethyl)-1H-pyrazol-4-yl]imidazo[1,2-α]pyrazin-8-yl]amino]benzamide;
2-chloro-N-[rac-(1R,3R)-3-aminocyclopentyl]-4-[[3-[3-(trifluoromethyl)-1H-pyrazol-4-yl]imidazo[1,2-α]pyrazin-8-yl]amino]benzamide;
N-(azetidin-3-yl)-4-[2-methyl-4-[[3-[3-(trifluoromethyl)-1H-pyrazol-4-yl]imidazo[1,2-α]pyrazin-8-yl]amino]benzoyl]piperazine-1-carboxamide;
N-(azetidin-3-ylmethyl)-4-[2-methyl-4-[[3-[3-(trifluoromethyl)-1H-pyrazol-4-yl]imidazo[1,2-α]pyrazin-8-yl]amino]benzoyl]piperazine-1-carboxamide;
2-chloro-N-piperidin-4-yl-4-[[3-[3-(trifluoromethyl)-1H-pyrazol-4-yl]imidazo[1,2-α]pyrazin-8-yl]amino]benzamide;
2-chloro-N-piperidin-3-yl-4-[[3-[3-(trifluoromethyl)-1H-pyrazol-4-yl]imidazo[1,2-α]pyrazin-8-yl]amino]benzamide;
4-[2-methyl-4-[[3-[3-(trifluoromethyl)-1H-pyrazol-4-yl]imidazo[1,2-α]pyrazin-8-yl]amino]benzoyl]-N-[(3S)-pyrrolidin-3-yl]piperazine-1-carboxamide;
4-[2-methyl-4-[[3-[3-(trifluoromethyl)-1H-pyrazol-4-yl]imidazo[1,2-α]pyrazin-8-yl]amino]benzoyl]-N-[(3R)-pyrrolidin-3-yl]piperazine-1-carboxamide;
N-(3-aminopropyl)-4-[2-methyl-4-[[3-[3-(trifluoromethyl)-1H-pyrazol-4-yl]imidazo[1,2-α]pyrazin-8-yl]amino]benzoyl]piperazine-1-carboxamide;
3-amino-1-[4-[2-methyl-4-[[3-[3-(trifluoromethyl)-1H-pyrazol-4-yl]imidazo[1,2-α]pyrazin-8-yl]amino]benzoyl]piperazin-1-yl]propan-1-one;
4-[4-[[3-[1-(cyanomethyl)-3-(trifluoromethyl)pyrazol-4-yl]imidazo[1,2-α]pyrazin-8-yl]amino]-2-methylbenzoyl]-N-[(3R,4R)-4-hydroxypyrrolidin-3-yl]piperazine-1-carboxamide;
4-amino-1-[4-[2-methyl-4-[[3-[3-(trifluoromethyl)-1H-pyrazol-4-yl]imidazo[1,2-α]pyrazin-8-yl]amino]benzoyl]piperazin-1-yl]butan-1-one;
2-[4-[8-[4-[4-[(3R,4R)-3-hydroxypiperidine-4-carbonyl]piperazine-1-carbonyl]-3-methylanilino]imidazo[1,2-α]pyrazin-3-yl]-3-(trifluoromethyl)pyrazol-1-yl]acetonitrile;
2-[4-[8-[4-[4-[(2S,4R)-4-hydroxypyrrolidine-2-carbonyl]piperazine-1-carbonyl]-3-methylanilino]imidazo[1,2-α]pyrazin-3-yl]-3-(trifluoromethyl)pyrazol-1-yl]acetonitrile;
2-[4-[8-[4-[4-[(3S,4S)-3-hydroxypiperidine-4-carbonyl]piperazine-1-carbonyl]-3-methylanilino]imidazo[1,2-α]pyrazin-3-yl]-3-(trifluoromethyl)pyrazol-1-yl]acetonitrile;
2-[4-[8-[4-[4-[(2S,4S)-4-hydroxy-4-methylpyrrolidine-2-carbonyl]piperazine-1-carbonyl]-3-methylanilino]imidazo[1,2-α]pyrazin-3-yl]-3-(trifluoromethyl)pyrazol-1-yl]acetonitrile;
2-[4-[8-[4-[4-(4-hydroxypiperidine-4-carbonyl)piperazine-1-carbonyl]-3-methylanilino]imidazo[1,2-α]pyrazin-3-yl]-3-(trifluoromethyl)pyrazol-1-yl]acetonitrile;
2-[4-[8-[4-[4-[(2R,4S)-4-hydroxypyrrolidine-2-carbonyl]piperazine-1-carbonyl]-3-methylanilino]imidazo[1,2-α]pyrazin-3-yl]-3-(trifluoromethyl)pyrazol-1-yl]acetonitrile;
N-(2-aminoethyl)-4-[2-methyl-4-[[3-[3-(trifluoromethyl)-1H-pyrazol-4-yl]imidazo[1,2-α]pyrazin-8-yl]amino]benzoyl]piperazine-1-carboxamide;
(2S,4R)—N-[3-[[2-chloro-4-[[3-[3-(trifluoromethyl)-1H-pyrazol-4-yl]imidazo[1,2-α]pyrazin-8-yl]amino]benzoyl]amino]propyl]-4-hydroxypyrrolidine-2-carboxamide;
N-[2-(2-aminoethoxy)ethyl]-4-[[3-[3-(difluoromethyl)-1H-pyrazol-4-yl]imidazo[1,2-α]pyrazin-8-yl]amino]-2-ethylbenzamide;
[2-chloro-4-[[3-[3-(trifluoromethyl)-1H-pyrazol-4-yl]imidazo[1,2-α]pyrazin-8-yl]amino]phenyl]-piperazin-1-ylmethanone;
(2S,4R)—N-[2-[[2-ethyl-4-[[3-[3-(trifluoromethyl)-1H-pyrazol-4-yl]imidazo[1,2-α]pyrazin-8-yl]amino]benzoyl]amino]ethyl]-4-hydroxypyrrolidine-2-carboxamide;
(2S,4R)—N-[2-[[2-chloro-4-[[3-[3-(trifluoromethyl)-1H-pyrazol-4-yl]imidazo[1,2-α]pyrazin-8-yl]amino]benzoyl]amino]ethyl]-4-hydroxypyrrolidine-2-carboxamide;
(2S,4R)—N-[3-[[2-ethyl-4-[[3-[3-(trifluoromethyl)-1H-pyrazol-4-yl]imidazo[1,2-α]pyrazin-8-yl]amino]benzoyl]amino]propyl]-4-hydroxypyrrolidine-2-carboxamide;
(2S,4S)-4-ethyl-N-[2-[[2-ethyl-4-[[3-[3-(trifluoromethyl)-1H-pyrazol-4-yl]imidazo[1,2-α]pyrazin-8-yl]amino]benzoyl]amino]ethyl]-4-hydroxypyrrolidine-2-carboxamide;
[4-(3-hydroxypiperidine-3-carbonyl)piperazin-1-yl]-[2-methyl-4-[[3-[3-(trifluoromethyl)-1H-pyrazol-4-yl]imidazo[1,2-α]pyrazin-8-yl]amino]phenyl]methanone;
(2S,4R)—N-[3-[[2-ethyl-4-[[3-[1-methyl-3-(trifluoromethyl)pyrazol-4-yl]imidazo[1,2-α]pyrazin-8-yl]amino]benzoyl]amino]propyl]-4-hydroxypyrrolidine-2-carboxamide;
[2-methyl-4-[[3-[3-(trifluoromethyl)-1H-pyrazol-4-yl]imidazo[1,2-α]pyrazin-8-yl]amino]phenyl]-[4-[rac-(3R,5R)-5-hydroxypiperidine-3-carbonyl]piperazin-1-yl]methanone;
4-[2-chloro-4-[[3-[3-(trifluoromethyl)-1H-pyrazol-4-yl]imidazo[1,2-α]pyrazin-8-yl]amino]benzoyl]-N-[(3S,4S)-4-hydroxypyrrolidin-3-yl]piperazine-1-carboxamide;
4-[2-chloro-4-[[3-[3-(trifluoromethyl)-1H-pyrazol-4-yl]imidazo[1,2-α]pyrazin-8-yl]amino]benzoyl]-N-[(3R,4R)-4-hydroxypyrrolidin-3-yl]piperazine-1-carboxamide;
(2S,4R)-4-hydroxy-N-[3-[[2-methyl-4-[[3-[3-(trifluoromethyl)-1H-pyrazol-4-yl]imidazo[1,2-α]pyrazin-8-yl]amino]benzoyl]amino]propyl]pyrrolidine-2-carboxamide;
[2-chloro-4-[[3-[3-(trifluoromethyl)-1H-pyrazol-4-yl]imidazo[1,2-α]pyrazin-8-yl]amino]phenyl]-[4-[(2S,4S)-4-hydroxy-4-methylpyrrolidine-2-carbonyl]piperazin-1-yl]methanone;
(2S,4R)-4-hydroxy-N-[2-[[2-methyl-4-[[3-[3-(trifluoromethyl)-1H-pyrazol-4-yl]imidazo[1,2-α]pyrazin-8-yl]amino]benzoyl]amino]ethyl]pyrrolidine-2-carboxamide;
(2S,4R)—N-[2-[[2-ethyl-4-[[3-[1-methyl-3-(trifluoromethyl)pyrazol-4-yl]imidazo[1,2-α]pyrazin-8-yl]amino]benzoyl]amino]ethyl]-4-hydroxypyrrolidine-2-carboxamide;
(2S,4S)-4-ethyl-N-[2-[[2-ethyl-4-[[3-[1-methyl-3-(trifluoromethyl)pyrazol-4-yl]imidazo[1,2-α]pyrazin-8-yl]amino]benzoyl]amino]ethyl]-4-hydroxypyrrolidine-2-carboxamide;
(2S,4S)—N-[2-[[2-ethyl-4-[[3-[1-methyl-3-(trifluoromethyl)pyrazol-4-yl]imidazo[1,2-α]pyrazin-8-yl]amino]benzoyl]amino]ethyl]-4-hydroxy-4-methylpyrrolidine-2-carboxamide;
[2-chloro-4-[[3-[3-(trifluoromethyl)-1H-pyrazol-4-yl]imidazo[1,2-α]pyrazin-8-yl]amino]phenyl]-[4-[(2S,4R)-4-hydroxypyrrolidine-2-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-(4-hydroxypiperidine-4-carbonyl)piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(3R,4R)-3-hydroxypiperidine-4-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(3S,4S)-3-hydroxypiperidine-4-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(2R,4S)-4-hydroxypyrrolidine-2-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(3R,4S)-3-hydroxypiperidine-4-carbonyl]piperazin-1-yl]methanone;
[2-chloro-4-[[3-[3-(trifluoromethyl)-1H-pyrazol-4-yl]imidazo[1,2-α]pyrazin-8-yl]amino]phenyl]-[4-[(3S,4R)-3-hydroxypiperidine-4-carbonyl]piperazin-1-yl]methanone;
(2S,4R)-4-hydroxy-N-[3-[[2-methyl-4-[[3-[1-methyl-3-(trifluoromethyl)pyrazol-4-yl]imidazo[1,2-α]pyrazin-8-yl]amino]benzoyl]amino]propyl]pyrrolidine-2-carboxamide;
(2S,4R)-4-hydroxy-N-[2-[[2-methyl-4-[[3-[1-methyl-3-(trifluoromethyl)pyrazol-4-yl]imidazo[1,2-α]pyrazin-8-yl]amino]benzoyl]amino]ethyl]pyrrolidine-2-carboxamide;
N-[(3R,4R)-4-hydroxypyrrolidin-3-yl]-4-[2-methyl-4-[[3-[3-(trifluoromethyl)-1H-pyrazol-4-yl]imidazo[1,2-α]pyrazin-8-yl]amino]benzoyl]piperazine-1-carboxamide;
N-[(3S,4S)-4-hydroxypyrrolidin-3-yl]-4-[2-methyl-4-[[3-[3-(trifluoromethyl)-1H-pyrazol-4-yl]imidazo[1,2-α]pyrazin-8-yl]amino]benzoyl]piperazine-1-carboxamide;
[4-[(2S,4R)-4-hydroxypyrrolidine-2-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-(4-hydroxypiperidine-4-carbonyl)piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-[(3R,4R)-3-hydroxypiperidine-4-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-[(2S,4S)-4-hydroxy-4-methylpyrrolidine-2-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-[(3S,4S)-3-hydroxypiperidine-4-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-[(3S,4R)-3-hydroxypiperidine-4-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-[(3R,4S)-3-hydroxypiperidine-4-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[4-[(2R,4S)-4-hydroxypyrrolidine-2-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[2-methyl-4-[[3-[3-(trifluoromethyl)-1H-pyrazol-4-yl]imidazo[1,2-α]pyrazin-8-yl]amino]phenyl]-[4-[rac-(3R,4R)-4-hydroxypiperidine-3-carbonyl]piperazin-1-yl]methanone;
[2-methyl-4-[[3-[3-(trifluoromethyl)-1H-pyrazol-4-yl]imidazo[1,2-α]pyrazin-8-yl]amino]phenyl]-[4-[rac-(3R,4S)-4-hydroxypiperidine-3-carbonyl]piperazin-1-yl]methanone;
N-[(3R,4R)-4-hydroxypyrrolidin-3-yl]-4-[2-methyl-4-[[3-[1-methyl-3-(trifluoromethyl)pyrazol-4-yl]imidazo[1,2-α]pyrazin-8-yl]amino]benzoyl]piperazine-1-carboxamide;
N-[(3S,4S)-4-hydroxypyrrolidin-3-yl]-4-[2-methyl-4-[[3-[1-methyl-3-(trifluoromethyl)pyrazol-4-yl]imidazo[1,2-α]pyrazin-8-yl]amino]benzoyl]piperazine-1-carboxamide;
[4-(4-hydroxypiperidine-4-carbonyl)piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(2S,4S)-4-hydroxy-4-methylpyrrolidine-2-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(2S,4R)-4-hydroxypyrrolidine-2-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(3R,4R)-3-hydroxypiperidine-4-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(3S,4S)-3-hydroxypiperidine-4-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(2R,4S)-4-hydroxypyrrolidine-2-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(3S,4R)-3-hydroxypiperidine-4-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(3R,4S)-3-hydroxypiperidine-4-carbonyl]piperazin-1-yl]-[2-methyl-4-[[3-[1-methyl-3-(trifluoromethyl)pyrazol-4-yl]imidazo[1,2-α]pyrazin-8-yl]amino]phenyl]methanone;
[4-[(2S,4R)-4-hydroxypyrrolidine-2-carbonyl]piperazin-1-yl]-[2-methyl-4-[[3-[3-(trifluoromethyl)-1H-pyrazol-4-yl]imidazo[1,2-α]pyrazin-8-yl]amino]phenyl]methanone;
[2-methyl-4-[[3-[3-(trifluoromethyl)-1H-pyrazol-4-yl]imidazo[1,2-α]pyrazin-8-yl]amino]phenyl]-piperazin-1-ylmethanone;
2-(4-(8-((4-(4-((1R,5S,6r)-3-azabicyclo[3.1.0]hexane-6-carbonyl)piperazine-1-carbonyl)-3-chlorophenyl)amino)imidazo[1,2-α]pyrazin-3-yl)-3-(trifluoromethyl)-1H-pyrazol-1-yl)acetonitrile;
N-(3-((1s,3s)-3-aminocyclobutane-1-carboxamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(3-((1R,3S)-3-(12-azaneyl)cyclopentane-1-carboxamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(3-((1S,3R)-3-aminocyclopentane-1-carboxamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(3-((1S,3R)-3-aminocyclopentane-1-carboxamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-(4-((3-(1-(cyanomethyl)-3-(trifluoromethyl)-1H-pyrazol-4-yl)imidazo[1,2-α]pyrazin-8-yl)amino)-2-ethylbenzamido)ethyl)piperidine-4-carboxamide;
N-(2-((1r,3r)-3-aminocyclobutane-1-carboxamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1r,3r)-3-aminocyclobutane-1-carboxamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(S)-3-amino-N-(2-(4-((3-(1-(2,2-difluoroethyl)-3-(trifluoromethyl)-1H-pyrazol-4-yl)imidazo[1,2-α]pyrazin-8-yl)amino)-2-ethylbenzamido)ethyl)pyrrolidine-1-carboxamide;
N-(2-((1R,3R)-3-aminocyclopentane-1-carboxamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-((1S,3S)-3-aminocyclopentane-1-carboxamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1S,3S)-3-aminocyclopentane-1-carboxamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(S)—N-(2-(2-aminopropanamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1R,3S)-3-aminocyclopentane-1-carboxamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1S,3R)-3-aminocyclopentane-1-carboxamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-((1S,3R)-3-aminocyclopentane-1-carboxamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1R,3S)-3-aminocyclopentane-1-carboxamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
cis-N-(2-((1s,3s)-3-aminocyclobutane-1-carboxamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
4-[[3-[1-(cyanomethyl)-3-(trifluoromethyl)pyrazol-4-yl]imidazo[1,2-α]pyrazin-8-yl]amino]-2-ethyl-N-(2-oxo-2-piperazin-1-yl-ethyl)benzamide;
N-[2-(4-amino-1-piperidyl)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
2-chloro-4-[[3-[1-(cyanomethyl)-3-(trifluoromethyl)pyrazol-4-yl]imidazo[1,2-α]pyrazin-8-yl]amino]-N-(2-oxo-2-piperazin-1-yl-ethyl)benzamide;
N-[2-(4-amino-1-piperidyl)-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
N-[2-(4-amino-1-piperidyl)-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
N-[2-[(3R)-3-aminopyrrolidin-1-yl]-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
4-[[3-[1-(cyanomethyl)-3-(trifluoromethyl)pyrazol-4-yl]imidazo[1,2-α]pyrazin-8-yl]amino]-N-[2-(2,6-diazaspiro[3.3]heptan-2-yl)-2-oxo-ethyl]-2-ethyl-benzamide;
N-[2-(3-amino-1-piperidyl)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
4-[[3-[1-(cyanomethyl)-3-(trifluoromethyl)pyrazol-4-yl]imidazo[1,2-α]pyrazin-8-yl]amino]-2-ethyl-N-[2-(3-methylpiperazin-1-yl)-2-oxo-ethyl]benzamide;
N-[2-(6-amino-2-azaspiro[3.3]heptan-2-yl)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
N-[2-[3-(aminomethyl)pyrrolidin-1-yl]-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
N-[2-[2-(aminomethyl)pyrrolidin-1-yl]-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
N-[2-(3-azabicyclo[3.1.0]hexan-6-ylamino)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
4-[[3-[1-(cyanomethyl)-3-(trifluoromethyl)pyrazol-4-yl]imidazo[1,2-α]pyrazin-8-yl]amino]-N-[2-(2,5-diazabicyclo[2.2.1]heptan-2-yl)-2-oxo-ethyl]-2-ethyl-benzamide;
N-(azetidin-3-ylmethyl)-2-ethyl-4-((3-(3-(trifluoromethyl)-1H-pyrazol-4-yl)imidazo[1,2-α]pyrazin-8-yl)amino)benzamide;
2-ethyl-N-(2-azaspiro[3.3]heptan-6-yl)-4-((3-(3-(trifluoromethyl)-1H-pyrazol-4-yl)imidazo[1,2-α]pyrazin-8-yl)amino)benzamide;
N-(azetidin-3-yl)-2-ethyl-4-((3-(3-(trifluoromethyl)-1H-pyrazol-4-yl)imidazo[1,2-α]pyrazin-8-yl)amino)benzamide;
(4-((1R,5S,6r)-3-azabicyclo[3.1.0]hexane-6-carbonyl)piperazin-1-yl)(2-chloro-4-((3-(3-(trifluoromethyl)-1H-pyrazol-4-yl)imidazo[1,2-α]pyrazin-8-yl)amino)phenyl)methanone;
(R)-3-amino-N-(2-(2-ethyl-4-((3-(3-(trifluoromethyl)-1H-pyrazol-4-yl)imidazo[1,2-α]pyrazin-8-yl)amino)benzamido)ethyl)pyrrolidine-1-carboxamide;
((3-aminopropyl)imino)(methyl)(2-methyl-4-((3-(3-(trifluoromethyl)-1H-pyrazol-4-yl)imidazo[1,2-α]pyrazin-8-yl)amino)phenyl)-16-sulfanone;
(4-((1R,5S,6r)-3-azabicyclo[3.1.0]hexane-6-carbonyl)piperazin-1-yl)(2-chloro-4-((3-(1-(2,2-difluoroethyl)-3-(trifluoromethyl)-1H-pyrazol-4-yl)imidazo[1,2-α]pyrazin-8-yl)amino)phenyl)methanone;
(R)-3-amino-N-(2-(4-((3-(1-(cyanomethyl)-3-(trifluoromethyl)-1H-pyrazol-4-yl)imidazo[1,2-α]pyrazin-8-yl)amino)-2-ethylbenzamido)ethyl)pyrrolidine-1-carboxamide;
N-[2-(azetidin-3-ylmethylamino)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
4-[[3-[1-(cyanomethyl)-3-(trifluoromethyl)pyrazol-4-yl]imidazo[1,2-α]pyrazin-8-yl]amino]-2-ethyl-N-[2-oxo-2-(pyrrolidin-2-ylmethylamino)ethyl]benzamide;
4-[[3-[1-(cyanomethyl)-3-(trifluoromethyl)pyrazol-4-yl]imidazo[1,2-α]pyrazin-8-yl]amino]-2-ethyl-N-[2-oxo-2-(pyrrolidin-3-ylmethylamino)ethyl]benzamide;
4-[[3-[1-(cyanomethyl)-3-(trifluoromethyl)pyrazol-4-yl]imidazo[1,2-α]pyrazin-8-yl]amino]-2-ethyl-N-[2-oxo-2-(4-piperidylmethylamino)ethyl]benzamide;
4-[[3-[1-(cyanomethyl)-3-(trifluoromethyl)pyrazol-4-yl]imidazo[1,2-α]pyrazin-8-yl]amino]-2-ethyl-N-[2-[(4-hydroxy-4-piperidyl)methylamino]-2-oxo-ethyl]benzamide;
N-[2-(2,6-diazaspiro[3.3]heptan-2-yl)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
N-[2-[3-(aminomethyl)pyrrolidin-1-yl]-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
N-[2-[2-(aminomethyl)pyrrolidin-1-yl]-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
N-[2-(3-azabicyclo[3.1.0]hexan-6-ylamino)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
N-[2-(azetidin-2-ylmethylamino)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
N-(3-((1s,3s)-3-aminocyclobutane-1-carboxamido)propyl)-2-ethyl-4-((3-(3-(trifluoromethyl)-1H-pyrazol-4-yl)imidazo[1,2-α]pyrazin-8-yl)amino)benzamide;
N-(3-((1r,3r)-3-aminocyclobutane-1-carboxamido)propyl)-2-ethyl-4-((3-(3-(trifluoromethyl)-1H-pyrazol-4-yl)imidazo[1,2-α]pyrazin-8-yl)amino)benzamide;
N-(3-((1R,3S)-3-aminocyclopentane-1-carboxamido)propyl)-2-ethyl-4-((3-(3-(trifluoromethyl)-1H-pyrazol-4-yl)imidazo[1,2-α]pyrazin-8-yl)amino)benzamide;
N-(3-((1S,3R)-3-aminocyclopentane-1-carboxamido)propyl)-2-ethyl-4-((3-(3-(trifluoromethyl)-1H-pyrazol-4-yl)imidazo[1,2-α]pyrazin-8-yl)amino)benzamide;
N-(2-(3-aminopropanamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-(4-aminobutanamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-[2-(2,5-diazabicyclo[2.2.1]heptan-2-yl)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
N-((1s,3s)-3-aminocyclobutyl)-4-((3-(1-(2,2-difluoroethyl)-3-(difluoromethyl)-1H-pyrazol-4-yl)imidazo[1,2-α]pyrazin-8-yl)amino)-2-ethylbenzamide;
N-(2-(4-aminobutanamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(3-((1s,3s)-3-aminocyclobutane-1-carboxamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(3-((1r,3r)-3-aminocyclobutane-1-carboxamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(3-((1R,3S)-3-aminocyclopentane-1-carboxamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-[2-(3-amino-1-piperidyl)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-(3-methylpiperazin-1-yl)-2-oxo-ethyl]benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-[(3-hydroxyazetidin-3-yl)methylamino]-2-oxo-ethyl]benzamide;
N-[2-(azetidin-3-ylmethylamino)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-oxo-2-(pyrrolidin-2-ylmethylamino)ethyl]benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-oxo-2-(pyrrolidin-3-ylmethylamino)ethyl]benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-oxo-2-(4-piperidylmethylamino)ethyl]benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-[(4-hydroxy-4-piperidyl)methylamino]-2-oxo-ethyl]benzamide;
(2S,4R)—N-((1s,3R)-3-(4-((3-(1-(2,2-difluoroethyl)-3-(difluoromethyl)-1H-pyrazol-4-yl)imidazo[1,2-α]pyrazin-8-yl)amino)-2-ethylbenzamido)cyclobutyl)-4-hydroxypyrrolidine-2-carboxamide;
N-(2-(2-ethyl-4-((3-(3-(trifluoromethyl)-1H-pyrazol-4-yl)imidazo[1,2-α]pyrazin-8-yl)amino)benzamido)ethyl)piperidine-4-carboxamide;
N-(2-(4-((3-(1-(2,2-difluoroethyl)-3-(trifluoromethyl)-1H-pyrazol-4-yl)imidazo[1,2-α]pyrazin-8-yl)amino)-2-ethylbenzamido)ethyl)piperidine-4-carboxamide;
(4-((3-(1-(2,2-difluoroethyl)-3-(trifluoromethyl)-1H-pyrazol-4-yl)imidazo[1,2-α]pyrazin-8-yl)amino)-2-ethylphenyl)(2,6-diazaspiro[3.4]octan-6-yl)methanone;
(3-aminoazetidin-1-yl)(4-((3-(1-(2,2-difluoroethyl)-3-(trifluoromethyl)-1H-pyrazol-4-yl)imidazo[1,2-α]pyrazin-8-yl)amino)-2-ethylphenyl)methanone;
N-(azetidin-3-ylmethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(3-amino-2-(chloromethyl)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(4-((3-(1-(2,2-difluoroethyl)-3-(trifluoromethyl)-1H-pyrazol-4-yl)imidazo[1,2-α]pyrazin-8-yl)amino)-2-ethylphenyl)(1,6-diazaspiro[3.3]heptan-1-yl)methanone;
(2-(aminomethyl)-2-(chloromethyl)azetidin-1-yl)(4-((3-(1-(2,2-difluoroethyl)-3-(trifluoromethyl)-1H-pyrazol-4-yl)imidazo[1,2-α]pyrazin-8-yl)amino)-2-ethylphenyl)methanone;
(2,5-diazabicyclo[2.2.1]heptan-2-yl)(4-((3-(1-(2,2-difluoroethyl)-3-(trifluoromethyl)-1H-pyrazol-4-yl)imidazo[1,2-α]pyrazin-8-yl)amino)-2-ethylphenyl)methanone;
N-(3-azabicyclo[3.1.0]hexan-6-yl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
4-((3-(1-(2,2-difluoroethyl)-3-(trifluoromethyl)-1H-pyrazol-4-yl)imidazo[1,2-α]pyrazin-8-yl)amino)-2-ethyl-N-(piperidin-3-yl)benzamide;
(4-((3-(1-(2,2-difluoroethyl)-3-(trifluoromethyl)-1H-pyrazol-4-yl)imidazo[1,2-α]pyrazin-8-yl)amino)-2-ethylphenyl)(1,6-diazaspiro[3.3]heptan-6-yl)methanone;
N-(azetidin-3-yl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-(3-aminopropanamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1r,3r)-3-aminocyclobutane-1-carboxamido)ethyl)-2-ethyl-4-((3-(3-(trifluoromethyl)-1H-pyrazol-4-yl)imidazo[1,2-α]pyrazin-8-yl)amino)benzamide;
4-amino-N-(2-(2-ethyl-4-((3-(3-(trifluoromethyl)-1H-pyrazol-4-yl)imidazo[1,2-α]pyrazin-8-yl)amino)benzamido)ethyl)piperidine-1-carboxamide;
N-(2-(3-(3-aminopropyl)ureido)ethyl)-2-ethyl-4-((3-(3-(trifluoromethyl)-1H-pyrazol-4-yl)imidazo[1,2-α]pyrazin-8-yl)amino)benzamide;
1-(4-(2-chloro-4-((3-(3-(trifluoromethyl)-1H-pyrazol-4-yl)imidazo[1,2-α]pyrazin-8-yl)amino)benzoyl)piperazin-1-yl)-2-(piperidin-4-yl)ethan-1-one;
(4-(2-aminoethyl)piperazin-1-yl)(2-chloro-4-((3-(3-(trifluoromethyl)-1H-pyrazol-4-yl)imidazo[1,2-α]pyrazin-8-yl)amino)phenyl)methanone;
2-(4-(8-((3-chloro-4-(4-(2-(piperidin-4-yl)acetyl)piperazine-1-carbonyl)phenyl)amino)imidazo[1,2-α]pyrazin-3-yl)-3-(trifluoromethyl)-1H-pyrazol-1-yl)acetonitrile;
2-(4-(8-((4-(4-((1R,3R)-3-aminocyclopentane-1-carbonyl)piperazine-1-carbonyl)-3-chlorophenyl)amino)imidazo[1,2-α]pyrazin-3-yl)-3-(trifluoromethyl)-1H-pyrazol-1-yl)acetonitrile;
(4-((1R,3R)-3-aminocyclopentane-1-carbonyl)piperazin-1-yl)(2-chloro-4-((3-(1-(2,2-difluoroethyl)-3-(trifluoromethyl)-1H-pyrazol-4-yl)imidazo[1,2-α]pyrazin-8-yl)amino)phenyl)methanone;
(4-((1S,3S)-3-aminocyclopentane-1-carbonyl)piperazin-1-yl)(2-chloro-4-((3-(3-(trifluoromethyl)-1H-pyrazol-4-yl)imidazo[1,2-α]pyrazin-8-yl)amino)phenyl)methanone;
(S)-3-amino-N-(2-(2-ethyl-4-((3-(3-(trifluoromethyl)-1H-pyrazol-4-yl)imidazo[1,2-α]pyrazin-8-yl)amino)benzamido)ethyl)pyrrolidine-1-carboxamide;
(S)-3-amino-N-(2-(4-((3-(1-(cyanomethyl)-3-(trifluoromethyl)-1H-pyrazol-4-yl)imidazo[1,2-α]pyrazin-8-yl)amino)-2-ethylbenzamido)ethyl)pyrrolidine-1-carboxamide;
1-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)-2-(piperidin-4-yl)ethan-1-one;
4-(2-chloro-4-((3-(1-(cyanomethyl)-3-(trifluoromethyl)-1H-pyrazol-4-yl)imidazo[1,2-α]pyrazin-8-yl)amino)benzoyl)-N-((3-hydroxyazetidin-3-yl)methyl)piperazine-1-carboxamide;
4-(2-chloro-4-((3-(1-(2,2-difluoroethyl)-3-(trifluoromethyl)-1H-pyrazol-4-yl)imidazo[1,2-α]pyrazin-8-yl)amino)benzoyl)-N-((3-hydroxyazetidin-3-yl)methyl)piperazine-1-carboxamide;
N-(azetidin-3-yl)-4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazine-1-carboxamide;
2-(4-(8-((4-(4-((1S,3S)-3-aminocyclopentane-1-carbonyl)piperazine-1-carbonyl)-3-chlorophenyl)amino)imidazo[1,2-α]pyrazin-3-yl)-3-(trifluoromethyl)-1H-pyrazol-1-yl)acetonitrile;
(4-((1S,3S)-3-aminocyclopentane-1-carbonyl)piperazin-1-yl)(2-chloro-4-((3-(1-(2,2-difluoroethyl)-3-(trifluoromethyl)-1H-pyrazol-4-yl)imidazo[1,2-α]pyrazin-8-yl)amino)phenyl)methanone;
N-((1s,3s)-3-aminocyclobutyl)-4-(2-chloro-4-((3-(1-(cyanomethyl)-3-(trifluoromethyl)-1H-pyrazol-4-yl)imidazo[1,2-α]pyrazin-8-yl)amino)benzoyl)piperazine-1-carboxamide;
N-((1s,3s)-3-aminocyclobutyl)-4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazine-1-carboxamide;
N-[2-[[(1R,3R)-3-aminocyclopentanecarbonyl]amino]ethyl]-2-ethyl-4-[[3-[3-(trifluoromethyl)-1H-pyrazol-4-yl]imidazo[1,2-α]pyrazin-8-yl]amino]benzamide;
N-(2-((1R,3R)-3-aminocyclopentane-1-carboxamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-((1S,3S)-3-aminocyclopentane-1-carboxamido)ethyl)-2-ethyl-4-((3-(3-(trifluoromethyl)-1H-pyrazol-4-yl)imidazo[1,2-α]pyrazin-8-yl)amino)benzamide;
4-((3-(1-((1-(cyanomethyl)-1H-pyrazol-3-yl)methyl)-3-(trifluoromethyl)-1H-pyrazol-4-yl)imidazo[1,2-α]pyrazin-8-yl)amino)-2-ethyl-N-methylbenzamide;
2-chloro-4-((3-(1-((1-(cyanomethyl)-1H-pyrazol-3-yl)methyl)-3-(trifluoromethyl)-1H-pyrazol-4-yl)imidazo[1,2-α]pyrazin-8-yl)amino)-N-methylbenzamide;
2-chloro-4-((3-(1-(2-(dimethylamino)ethyl)-3-(trifluoromethyl)-1H-pyrazol-4-yl)imidazo[1,2-α]pyrazin-8-yl)amino)-N-methylbenzamide;
4-((3-(1-(1-benzylpiperidin-4-yl)-3-(trifluoromethyl)-1H-pyrazol-4-yl)imidazo[1,2-α]pyrazin-8-yl)amino)-2-chloro-N-methylbenzamide;
4-((3-(1-((1-benzylpyrrolidin-3-yl)methyl)-3-(trifluoromethyl)-1H-pyrazol-4-yl)imidazo[1,2-α]pyrazin-8-yl)amino)-2-chloro-N-methylbenzamide;
4-((3-(1-(3-(4-(aminomethyl)phenyl)propyl)-3-(trifluoromethyl)-1H-pyrazol-4-yl)imidazo[1,2-α]pyrazin-8-yl)amino)-2-chloro-N-methylbenzamide;
4-((3-(1-(3-(2-aminoethyl)benzyl)-3-(trifluoromethyl)-1H-pyrazol-4-yl)imidazo[1,2-α]pyrazin-8-yl)amino)-2-chloro-N-methylbenzamide;
N-[4-[N-(3-aminopropyl)-S-methyl-sulfonimidoyl]-3-methyl-phenyl]-3-[1-(cyclopropylmethyl)-3-(trifluoromethyl)pyrazol-4-yl]imidazo[1,2-α]pyrazin-8-amine;
2-[4-[8-[4-[N-(3-aminopropyl)-S-methyl-sulfonimidoyl]-3-methyl-anilino]imidazo[1,2-α]pyrazin-3-yl]-3-(trifluoromethyl)pyrazol-1-yl]acetonitrile;
N-(2-aminoethyl)-2-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)acetamide;
N-(azetidin-3-yl)-2-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)acetamide;
(S)-2-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)-N-(pyrrolidin-3-yl)acetamide;
(R)-2-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)-N-(pyrrolidin-3-yl)acetamide;
N-(2-aminoethyl)-2-(4-(2-chloro-4-((3-(1-(cyanomethyl)-3-(trifluoromethyl)-1H-pyrazol-4-yl)imidazo[1,2-α]pyrazin-8-yl)amino)benzoyl)piperazin-1-yl)acetamide;
N-(3-aminopropyl)-2-(4-(2-chloro-4-((3-(1-(cyanomethyl)-3-(trifluoromethyl)-1H-pyrazol-4-yl)imidazo[1,2-α]pyrazin-8-yl)amino)benzoyl)piperazin-1-yl)acetamide;
N-(azetidin-3-yl)-2-(4-(2-chloro-4-((3-(1-(cyanomethyl)-3-(trifluoromethyl)-1H-pyrazol-4-yl)imidazo[1,2-α]pyrazin-8-yl)amino)benzoyl)piperazin-1-yl)acetamide;
(S)-2-(4-(2-chloro-4-((3-(1-(cyanomethyl)-3-(trifluoromethyl)-1H-pyrazol-4-yl)imidazo[1,2-α]pyrazin-8-yl)amino)benzoyl)piperazin-1-yl)-N-(pyrrolidin-3-yl)acetamide;
N-(3-aminopropyl)-2-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)acetamide;
2-(4-(8-((3-chloro-4-(4-(2-oxo-2-(piperazin-1-yl)ethyl)piperazine-1-carbonyl)phenyl)amino)imidazo[1,2-α]pyrazin-3-yl)-3-(trifluoromethyl)-1H-pyrazol-1-yl)acetonitrile;
2-(4-(2-chloro-4-((3-(1-(2,2-difluoroethyl)-3-(trifluoromethyl)-1H-pyrazol-4-yl)imidazo[1,2-α]pyrazin-8-yl)amino)benzoyl)piperazin-1-yl)-1-(piperazin-1-yl)ethan-1-one;
N-(2-(3-aminopropoxy)ethyl)-4-((3-(1-cyclobutyl-3-(trifluoromethyl)-1H-pyrazol-4-yl)imidazo[1,2-α]pyrazin-8-yl)amino)-2-ethylbenzamide;
N-(2-(3-aminopropoxy)ethyl)-2-ethyl-4-((3-(1-(2-fluoroethyl)-3-(trifluoromethyl)-1H-pyrazol-4-yl)imidazo[1,2-α]pyrazin-8-yl)amino)benzamide;
N-(2-(2-aminoacetamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-(3-aminopropoxy)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
N-(2-(3-aminopropoxy)ethyl)-2-ethyl-4-((3-(3-(trifluoromethyl)-1H-pyrazol-4-yl)imidazo[1,2-α]pyrazin-8-yl)amino)benzamide;
N-(2-(2-aminoethoxy)ethyl)-2-ethyl-4-((3-(1-(3-methylbut-2-en-1-yl)-3-(trifluoromethyl)-1H-pyrazol-4-yl)imidazo[1,2-α]pyrazin-8-yl)amino)benzamide;
N-(6-aminohexyl)-4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamide;
(S)—N-(2-(2-aminopropanamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
2-ethyl-4-((3-(1-(2-fluoroethyl)-3-(trifluoromethyl)-1H-pyrazol-4-yl)imidazo[1,2-α]pyrazin-8-yl)amino)-N-(2-(2-(pyrrolidin-1-yl)ethoxy)ethyl)benzamide;
N-(2-(3-aminopropoxy)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
4-((3-(1-cyclobutyl-3-(trifluoromethyl)-1H-pyrazol-4-yl)imidazo[1,2-α]pyrazin-8-yl)amino)-2-ethyl-N-(2-(2-(pyrrolidin-1-yl)ethoxy)ethyl)benzamide;
N-(2-(2-aminoacetamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
4-((3-(1-(2,2-difluoroethyl)-3-(trifluoromethyl)-1H-pyrazol-4-yl)imidazo[1,2-α]pyrazin-8-yl)amino)-2-ethyl-N-(2-(2-(pyrrolidin-1-yl)ethoxy)ethyl)benzamide;
N-(2-(2-aminoethoxy)ethyl)-2-ethyl-4-((3-(1-(2-(trifluoromethoxy)ethyl)-3-(trifluoromethyl)-1H-pyrazol-4-yl)imidazo[1,2-α]pyrazin-8-yl)amino)benzamide;
N-((2-aminocyclopropyl)methyl)-4-((3-(1-(cyanomethyl)-3-(trifluoromethyl)-1H-pyrazol-4-yl)imidazo[1,2-α]pyrazin-8-yl)amino)-2-ethylbenzamide;
cis-N-(2-((1s,3s)-3-aminocyclobutane-1-carboxamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(R)—N-(2-(2-aminopropanamido)ethyl)-4-((3-(1-(cyanomethyl)-3-(trifluoromethyl)-1H-pyrazol-4-yl)imidazo[1,2-α]pyrazin-8-yl)amino)-2-ethylbenzamide;
(R)—N-(3-(2-aminopropanamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
(S)—N-(3-(4-((3-(1-(2,2-difluoroethyl)-3-(trifluoromethyl)-1H-pyrazol-4-yl)imidazo[1,2-α]pyrazin-8-yl)amino)-2-ethylbenzamido)propyl)pyrrolidine-2-carboxamide;
(S)—N-(3-(2-aminopropanamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(4-(4-((3-(1-allyl-3-(trifluoromethyl)-1H-pyrazol-4-yl)imidazo[1,2-α]pyrazin-8-yl)amino)-2-fluoro-6-methylbenzoyl)piperazin-1-yl)((2R,5S)-5-hydroxypyrrolidin-2-yl)methanone;
N-(3-(3-aminopropanamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(R)-3-amino-N-(3-(2-ethyl-4-((3-(3-(trifluoromethyl)-1H-pyrazol-4-yl)imidazo[1,2-α]pyrazin-8-yl)amino)benzamido)propyl)pyrrolidine-1-carboxamide;
(R)-3-amino-N-(3-(4-((3-(1-(2,2-difluoroethyl)-3-(trifluoromethyl)-1H-pyrazol-4-yl)imidazo[1,2-α]pyrazin-8-yl)amino)-2-ethylbenzamido)propyl)pyrrolidine-1-carboxamide;
(R)—N-(3-(2-aminopropanamido)propyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
(S)—N-(3-(2-ethyl-4-((3-(3-(trifluoromethyl)-1H-pyrazol-4-yl)imidazo[1,2-α]pyrazin-8-yl)amino)benzamido)propyl)pyrrolidine-2-carboxamide;
(S)-(2-chloro-4-((3-(1-(2,2-difluoroethyl)-3-(trifluoromethyl)-1H-pyrazol-4-yl)imidazo[1,2-α]pyrazin-8-yl)amino)phenyl)(6-prolyl-2,6-diazaspiro[3.3]heptan-2-yl)methanone;
(S)-2-(4-(8-((3-chloro-4-(6-prolyl-2,6-diazaspiro[3.3]heptane-2-carbonyl)phenyl)amino)imidazo[1,2-α]pyrazin-3-yl)-3-(trifluoromethyl)-1H-pyrazol-1-yl)acetonitrile;
(2S,4R)—N-(2-(4-((3-(1-(cyanomethyl)-3-(trifluoromethyl)-1H-pyrazol-4-yl)imidazo[1,2-α]pyrazin-8-yl)amino)-2-ethylbenzamido)ethyl)-4-hydroxypyrrolidine-2-carboxamide;
4-[[3-[1-(2,2-difluoroethyl)-3-(trifluoromethyl)pyrazol-4-yl]imidazo[1,2-α]pyrazin-8-yl]amino]-2-ethyl-N-[2-oxo-2-[[(3S)-pyrrolidin-3-yl]amino]ethyl]benzamide;
N-(2-(3-aminopropoxy)ethyl)-4-((3-(1-(1-cyanoethyl)-3-(trifluoromethyl)-1H-pyrazol-4-yl)imidazo[1,2-α]pyrazin-8-yl)amino)-2-ethylbenzamide;
N-((2-aminocyclopropyl)methyl)-2-ethyl-4-((3-(1-(2-fluoroethyl)-3-(trifluoromethyl)-1H-pyrazol-4-yl)imidazo[1,2-α]pyrazin-8-yl)amino)benzamide;
N-((2-aminocyclopropyl)methyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-[2-[(3-aminocyclobutyl)amino]-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
(R)—N-(2-(2-aminopropanamido)ethyl)-4-((3-(1-(2,2-difluoroethyl)-3-(trifluoromethyl)-1H-pyrazol-4-yl)imidazo[1,2-α]pyrazin-8-yl)amino)-2-ethylbenzamide;
N-(2-(2-aminoethoxy)ethyl)-2-ethyl-4-((3-(1-(3,3,3-trifluoro-2-hydroxypropyl)-3-(trifluoromethyl)-1H-pyrazol-4-yl)imidazo[1,2-α]pyrazin-8-yl)amino)benzamide;
4-((3-(1-(1-cyanoethyl)-3-(trifluoromethyl)-1H-pyrazol-4-yl)imidazo[1,2-α]pyrazin-8-yl)amino)-2-ethyl-N-(2-(2-(pyrrolidin-1-yl)ethoxy)ethyl)benzamide;
(4-(2-fluoro-6-methyl-4-((3-(1-(prop-2-yn-1-yl)-3-(trifluoromethyl)-1H-pyrazol-4-yl)imidazo[1,2-α]pyrazin-8-yl)amino)benzoyl)piperazin-1-yl)((2R,5S)-5-hydroxypyrrolidin-2-yl)methanone;
2-chloro-4-[[3-[1-(cyanomethyl)-3-(trifluoromethyl)pyrazol-4-yl]imidazo[1,2-α]pyrazin-8-yl]amino]-N-[(1R)-1-methyl-2-oxo-2-piperazin-1-yl-ethyl]benzamide;
4-[[3-[1-(2,2-difluoroethyl)-3-(trifluoromethyl)pyrazol-4-yl]imidazo[1,2-α]pyrazin-8-yl]amino]-2-ethyl-N-[2-oxo-2-[[(3R)-pyrrolidin-3-yl]amino]ethyl]benzamide;
(S)—N—((R)-2-(4-((3-(1-(2,2-difluoroethyl)-3-(trifluoromethyl)-1H-pyrazol-4-yl)imidazo[1,2-α]pyrazin-8-yl)amino)-2-ethylbenzamido)propyl)pyrrolidine-2-carboxamide;
2-amino-1-(4-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzoyl)piperazin-1-yl)ethan-1-one;
2-amino-1-(6-(2-chloro-4-((3-(1-(2,2-difluoroethyl)-3-(trifluoromethyl)-1H-pyrazol-4-yl)imidazo[1,2-α]pyrazin-8-yl)amino)benzoyl)-2,6-diazaspiro[3.3]heptan-2-yl)ethan-1-one;
(S)—N-(3-(2-aminopropanamido)propyl)-2-ethyl-4-((3-(3-(trifluoromethyl)-1H-pyrazol-4-yl)imidazo[1,2-α]pyrazin-8-yl)amino)benzamide;
(1R,5S,6r)-N—((R)-2-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamido)propyl)-3-azabicyclo[3.1.0]hexane-6-carboxamide;
(S)—N—((R)-2-(4-((3-(1-(cyanomethyl)-3-(trifluoromethyl)-1H-pyrazol-4-yl)imidazo[1,2-α]pyrazin-8-yl)amino)-2-ethylbenzamido)propyl)pyrrolidine-2-carboxamide;
N-(2-(2-aminoethoxy)ethyl)-4-((3-(1-(3-chloro-3-methylbutyl)-3-(trifluoromethyl)-1H-pyrazol-4-yl)imidazo[1,2-α]pyrazin-8-yl)amino)-2-ethylbenzamide;
N-[2-[(3S)-3-aminopyrrolidin-1-yl]-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
2-amino-1-(6-(2-chloro-4-((3-(3-(trifluoromethyl)-1H-pyrazol-4-yl)imidazo[1,2-α]pyrazin-8-yl)amino)benzoyl)-2,6-diazaspiro[3.3]heptan-2-yl)ethan-1-one;
2-(4-(8-((3-chloro-4-(6-glycyl-2,6-diazaspiro[3.3]heptane-2-carbonyl)phenyl)amino)imidazo[1,2-α]pyrazin-3-yl)-3-(trifluoromethyl)-1H-pyrazol-1-yl)acetonitrile;
N-[2-[(3-aminocyclobutyl)amino]-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
4-((3-(1-cyclobutyl-3-(trifluoromethyl)-1H-pyrazol-4-yl)imidazo[1,2-α]pyrazin-8-yl)amino)-2-ethyl-N-(2-(2-morpholinoethoxy)ethyl)benzamide;
(R)—N-(1-aminopropan-2-yl)-2-fluoro-6-methyl-4-((3-(1-(prop-2-yn-1-yl)-3-(trifluoromethyl)-1H-pyrazol-4-yl)imidazo[1,2-α]pyrazin-8-yl)amino)benzamide;
N-[2-[(3R)-3-aminopyrrolidin-1-yl]-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
(S)-2-amino-1-(4-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzoyl)piperazin-1-yl)propan-1-one;
N-[(1R)-2-[(3S)-3-aminopyrrolidin-1-yl]-1-methyl-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
N-[2-(4-amino-1-piperidyl)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
(2S,4R)—N-(2-(4-((3-(1-(2,2-difluoroethyl)-3-(trifluoromethyl)-1H-pyrazol-4-yl)imidazo[1,2-α]pyrazin-8-yl)amino)-2-ethylbenzamido)ethyl)-4-hydroxypyrrolidine-2-carboxamide;
4-[[3-[1-(2,2-difluoroethyl)-3-(trifluoromethyl)pyrazol-4-yl]imidazo[1,2-α]pyrazin-8-yl]amino]-N-[2-[2-(dimethylamino)ethylamino]-2-oxo-ethyl]-2-ethyl-benzamide;
N—((R)-1-((1s,3S)-3-aminocyclobutane-1-carboxamido)propan-2-yl)-4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamide;
N-(2-(3-aminopropoxy)ethyl)-4-((3-(1-(3,3-difluorocyclobutyl)-3-(trifluoromethyl)-1H-pyrazol-4-yl)imidazo[1,2-α]pyrazin-8-yl)amino)-2-ethylbenzamide;
N-((2-aminocyclopropyl)methyl)-4-((3-(1-cyclobutyl-3-(trifluoromethyl)-1H-pyrazol-4-yl)imidazo[1,2-α]pyrazin-8-yl)amino)-2-ethylbenzamide;
2-ethyl-N-(2-(2-(pyrrolidin-1-yl)ethoxy)ethyl)-4-((3-(3-(trifluoromethyl)-1H-pyrazol-4-yl)imidazo[1,2-α]pyrazin-8-yl)amino)benzamide;
(R)—N-(1-(2-aminoacetamido)propan-2-yl)-4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamide;
N-[(1R)-2-[(3R)-3-aminopyrrolidin-1-yl]-1-methyl-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
N-[2-[(3-aminocyclobutyl)amino]-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
2-(4-(8-((4-(4-((1R,5S,6r)-3-azabicyclo[3.1.0]hexane-6-carbonyl)piperazine-1-carbonyl)-3-fluoro-5-methylphenyl)amino)imidazo[1,2-α]pyrazin-3-yl)-3-(trifluoromethyl)-1H-pyrazol-1-yl)acetonitrile;
(R)—N-(3-(2-aminopropanamido)propyl)-2-ethyl-4-((3-(3-(trifluoromethyl)-1H-pyrazol-4-yl)imidazo[1,2-α]pyrazin-8-yl)amino)benzamide;
4-[[3-[1-(cyanomethyl)-3-(trifluoromethyl)pyrazol-4-yl]imidazo[1,2-α]pyrazin-8-yl]amino]-2-ethyl-N-[(1R)-1-methyl-2-oxo-2-piperazin-1-yl-ethyl]benzamide;
4-[[3-[1-(2,2-difluoroethyl)-3-(trifluoromethyl)pyrazol-4-yl]imidazo[1,2-α]pyrazin-8-yl]amino]-2-ethyl-N-(2-oxo-2-piperazin-1-yl-ethyl)benzamide;
(R)-4-((3-(1-allyl-3-(trifluoromethyl)-1H-pyrazol-4-yl)imidazo[1,2-α]pyrazin-8-yl)amino)-N-(1-aminopropan-2-yl)-2-fluoro-6-methylbenzamide;
N-[2-[(3S)-3-aminopyrrolidin-1-yl]-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
2-(4-(8-((4-(4-(L-alanyl)piperazine-1-carbonyl)-3-fluoro-5-methylphenyl)amino)imidazo[1,2-α]pyrazin-3-yl)-3-(trifluoromethyl)-1H-pyrazol-1-yl)acetonitrile;
N-[(1R)-2-[(3-aminocyclobutyl)amino]-1-methyl-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
(2S,4R)—N—((R)-2-(4-((3-(1-(2,2-difluoroethyl)-3-(trifluoromethyl)-1H-pyrazol-4-yl)imidazo[1,2-α]pyrazin-8-yl)amino)-2-ethylbenzamido)propyl)-4-hydroxypyrrolidine-2-carboxamide;
(R)-3-amino-N-(3-(4-((3-(1-(cyanomethyl)-3-(trifluoromethyl)-1H-pyrazol-4-yl)imidazo[1,2-α]pyrazin-8-yl)amino)-2-ethylbenzamido)propyl)pyrrolidine-1-carboxamide;
N-[2-[(3R)-3-aminopyrrolidin-1-yl]-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
N-[2-[(3R)-3-aminopyrrolidin-1-yl]-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
(S)-(2-chloro-4-((3-(3-(trifluoromethyl)-1H-pyrazol-4-yl)imidazo[1,2-α]pyrazin-8-yl)amino)phenyl)(6-prolyl-2,6-diazaspiro[3.3]heptan-2-yl)methanone;
(R)—N-(1-(2-aminoacetamido)propan-2-yl)-4-((3-(1-(cyanomethyl)-3-(trifluoromethyl)-1H-pyrazol-4-yl)imidazo[1,2-α]pyrazin-8-yl)amino)-2-fluoro-6-methylbenzamide;
N-[(1R)-2-(4-amino-1-piperidyl)-1-methyl-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
N-(2-(2-aminoacetamido)ethyl)-2-ethyl-4-((3-(3-(trifluoromethyl)-1H-pyrazol-4-yl)imidazo[1,2-α]pyrazin-8-yl)amino)benzamide;
(4-((1R,5S,6r)-3-azabicyclo[3.1.0]hexane-6-carbonyl)piperazin-1-yl)(4-((3-(1-(2,2-difluoroethyl)-3-(trifluoromethyl)-1H-pyrazol-4-yl)imidazo[1,2-α]pyrazin-8-yl)amino)-2-fluoro-6-methylphenyl)methanone;
2-chloro-4-[[3-[1-(cyanomethyl)-3-(trifluoromethyl)pyrazol-4-yl]imidazo[1,2-α]pyrazin-8-yl]amino]-N-[2-[2-(dimethylamino)ethylamino]-2-oxo-ethyl]benzamide;
(S)-2-amino-1-(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)propan-1-one;
2-(4-(8-((3-fluoro-4-(4-glycylpiperazine-1-carbonyl)-5-methylphenyl)amino)imidazo[1,2-α]pyrazin-3-yl)-3-(trifluoromethyl)-1H-pyrazol-1-yl)acetonitrile;
(R)—N-(2-(2-aminopropanamido)ethyl)-2-ethyl-4-((3-(3-(trifluoromethyl)-1H-pyrazol-4-yl)imidazo[1,2-α]pyrazin-8-yl)amino)benzamide;
N-[(1R)-2-(4-amino-1-piperidyl)-1-methyl-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
(4-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzoyl)piperazin-1-yl)((2S,4R)-4-hydroxypyrrolidin-2-yl)methanone;
2-(4-(8-((4-(4-((1s,3s)-3-aminocyclobutane-1-carbonyl)piperazine-1-carbonyl)-3-fluoro-5-methylphenyl)amino)imidazo[1,2-α]pyrazin-3-yl)-3-(trifluoromethyl)-1H-pyrazol-1-yl)acetonitrile;
N-((2-aminocyclopropyl)methyl)-4-((3-(1-(1-cyanoethyl)-3-(trifluoromethyl)-1H-pyrazol-4-yl)imidazo[1,2-α]pyrazin-8-yl)amino)-2-ethylbenzamide;
(4-((1s,3s)-3-aminocyclobutane-1-carbonyl)piperazin-1-yl)(4-((3-(1-(2,2-difluoroethyl)-3-(trifluoromethyl)-1H-pyrazol-4-yl)imidazo[1,2-α]pyrazin-8-yl)amino)-2-fluoro-6-methylphenyl)methanone;
N-(3-(3-aminopropanamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
2-(4-(8-((3-fluoro-5-methyl-4-(4-(piperidine-4-carbonyl)piperazine-1-carbonyl)phenyl)amino)imidazo[1,2-α]pyrazin-3-yl)-3-(trifluoromethyl)-1H-pyrazol-1-yl)acetonitrile;
(R)—N-(1-aminopropan-2-yl)-4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamide;
(R)—N-(2-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamido)propyl)piperidine-4-carboxamide;
4-[[3-[1-(cyanomethyl)-3-(trifluoromethyl)pyrazol-4-yl]imidazo[1,2-α]pyrazin-8-yl]amino]-2-methyl-N-(2-oxo-2-piperazin-1-yl-ethyl)benzamide;
(4-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzoyl)piperazin-1-yl)(piperidin-4-yl)methanone;
2-ethyl-N-(3-(2-(2,2,2-trifluoroacetamido)acetamido)propyl)-4-((3-(3-(trifluoromethyl)-1H-pyrazol-4-yl)imidazo[1,2-α]pyrazin-8-yl)amino)benzamide;
N—((R)-1-((1s,3S)-3-aminocyclobutane-1-carboxamido)propan-2-yl)-4-((3-(1-(cyanomethyl)-3-(trifluoromethyl)-1H-pyrazol-4-yl)imidazo[1,2-α]pyrazin-8-yl)amino)-2-fluoro-6-methylbenzamide;
2-chloro-4-[[3-[1-(cyanomethyl)-3-(trifluoromethyl)pyrazol-4-yl]imidazo[1,2-α]pyrazin-8-yl]amino]-N-[(1R)-1-methyl-2-oxo-2-[[(3S)-pyrrolidin-3-yl]amino]ethyl]benzamide;
N-[2-[(3S)-3-aminopyrrolidin-1-yl]-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
(2S,4R)—N—((R)-2-(4-((3-(1-(cyanomethyl)-3-(trifluoromethyl)-1H-pyrazol-4-yl)imidazo[1,2-α]pyrazin-8-yl)amino)-2-ethylbenzamido)propyl)-4-hydroxypyrrolidine-2-carboxamide;
N-[2-(3-aminoazetidin-1-yl)-2-oxo-ethyl]-2-chloro-4-[[3-[1-(cyanomethyl)-3-(trifluoromethyl)pyrazol-4-yl]imidazo[1,2-α]pyrazin-8-yl]amino]benzamide;
(4-(2-fluoro-4-((3-(1-(2-fluoroethyl)-3-(trifluoromethyl)-1H-pyrazol-4-yl)imidazo[1,2-α]pyrazin-8-yl)amino)-6-methylbenzoyl)piperazin-1-yl)((2R,5S)-5-hydroxypyrrolidin-2-yl)methanone;
2-(4-(8-((3-fluoro-4-(4-((2R,5S)-5-hydroxypyrrolidine-2-carbonyl)piperazine-1-carbonyl)-5-methylphenyl)amino)imidazo[1,2-α]pyrazin-3-yl)-3-(trifluoromethyl)-1H-pyrazol-1-yl)acetonitrile;
N-[2-(3-aminoazetidin-1-yl)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
4-((3-(1-(2,2-difluoroethyl)-3-(trifluoromethyl)-1H-pyrazol-4-yl)imidazo[1,2-α]pyrazin-8-yl)amino)-2-ethyl-N-(2-(2-morpholinoethoxy)ethyl)benzamide;
(S)—N-(2-(2-aminopropanamido)ethyl)-2-ethyl-4-((3-(3-(trifluoromethyl)-1H-pyrazol-4-yl)imidazo[1,2-α]pyrazin-8-yl)amino)benzamide;
(R)—N-(1-aminopropan-2-yl)-4-((3-(1-(cyanomethyl)-3-(trifluoromethyl)-1H-pyrazol-4-yl)imidazo[1,2-α]pyrazin-8-yl)amino)-2-fluoro-6-methylbenzamide;
(1R,5S,6r)-N—((R)-2-(4-((3-(1-(cyanomethyl)-3-(trifluoromethyl)-1H-pyrazol-4-yl)imidazo[1,2-α]pyrazin-8-yl)amino)-2-fluoro-6-methylbenzamido)propyl)-3-azabicyclo[3.1.0]hexane-6-carboxamide;
2-(4-(8-((3-fluoro-4-(4-((2S,4R)-4-hydroxypyrrolidine-2-carbonyl)piperazine-1-carbonyl)-5-methylphenyl)amino)imidazo[1,2-α]pyrazin-3-yl)-3-(trifluoromethyl)-1H-pyrazol-1-yl)acetonitrile;
N—((R)-1-((S)-2-aminopropanamido)propan-2-yl)-4-((3-(1-(cyanomethyl)-3-(trifluoromethyl)-1H-pyrazol-4-yl)imidazo[1,2-α]pyrazin-8-yl)amino)-2-fluoro-6-methylbenzamide;
2-chloro-4-[[3-[1-(cyanomethyl)-3-(trifluoromethyl)pyrazol-4-yl]imidazo[1,2-α]pyrazin-8-yl]amino]-N-[2-oxo-2-[[(3R)-pyrrolidin-3-yl]amino]ethyl]benzamide;
N-(2-((1S,3R)-3-aminocyclopentane-1-carboxamido)ethyl)-2-ethyl-4-((3-(3-(trifluoromethyl)-1H-pyrazol-4-yl)imidazo[1,2-α]pyrazin-8-yl)amino)benzamide;
N-[(1R)-2-[(3-aminocyclobutyl)amino]-1-methyl-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
4-[[3-[1-(cyanomethyl)-3-(trifluoromethyl)pyrazol-4-yl]imidazo[1,2-α]pyrazin-8-yl]amino]-2-methyl-N-[(1R)-1-methyl-2-oxo-2-piperazin-1-yl-ethyl]benzamide;
(2S,4R)—N-(3-(4-((3-(1-(2,2-difluoroethyl)-3-(trifluoromethyl)-1H-pyrazol-4-yl)imidazo[1,2-α]pyrazin-8-yl)amino)-2-ethylbenzamido)propyl)-4-hydroxypyrrolidine-2-carboxamide;
N-[2-(2-aminoethylamino)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)(piperidin-4-yl)methanone;
2-chloro-4-[[3-[1-(cyanomethyl)-3-(trifluoromethyl)pyrazol-4-yl]imidazo[1,2-α]pyrazin-8-yl]amino]-N-[2-oxo-2-[[(3S)-pyrrolidin-3-yl]amino]ethyl]benzamide;
N-[(1R)-2-[(3R)-3-aminopyrrolidin-1-yl]-1-methyl-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
N-[2-(3-aminoazetidin-1-yl)-2-oxo-ethyl]-4-[[3-[1-(2,2-difluoroethyl)-3-(trifluoromethyl)pyrazol-4-yl]imidazo[1,2-α]pyrazin-8-yl]amino]-2-ethyl-benzamide;
(4-((1r,3r)-3-aminocyclobutane-1-carbonyl)piperazin-1-yl)(4-((3-(3-(difluoromethyl)-1-(prop-2-yn-1-yl)-1H-pyrazol-4-yl)imidazo[1,2-α]pyrazin-8-yl)amino)-2-ethylphenyl)methanone;
(R)—N-(1-aminopropan-2-yl)-2-fluoro-4-((3-(1-(2-fluoroethyl)-3-(trifluoromethyl)-1H-pyrazol-4-yl)imidazo[1,2-α]pyrazin-8-yl)amino)-6-methylbenzamide;
N-[(1R)-2-[(3S)-3-aminopyrrolidin-1-yl]-1-methyl-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
2-ethyl-4-((3-(1-(2-fluoroethyl)-3-(trifluoromethyl)-1H-pyrazol-4-yl)imidazo[1,2-α]pyrazin-8-yl)amino)-N-(2-(2-morpholinoethoxy)ethyl)benzamide;
4-[[3-[1-(cyanomethyl)-3-(trifluoromethyl)pyrazol-4-yl]imidazo[1,2-α]pyrazin-8-yl]amino]-2-methyl-N-[(1R)-1-methyl-2-oxo-2-[[(3S)-pyrrolidin-3-yl]amino]ethyl]benzamide;
N-[2-[(3S)-3-aminopyrrolidin-1-yl]-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
N-(2-((1s,3s)-3-aminocyclobutane-1-carboxamido)ethyl)-2-ethyl-4-((3-(3-(trifluoromethyl)-1H-pyrazol-4-yl)imidazo[1,2-α]pyrazin-8-yl)amino)benzamide;
(R)—N-(2-(4-((3-(1-(cyanomethyl)-3-(trifluoromethyl)-1H-pyrazol-4-yl)imidazo[1,2-α]pyrazin-8-yl)amino)-2-fluoro-6-methylbenzamido)propyl)piperidine-4-carboxamide;
(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)(piperidin-4-yl)methanone;
(4-((1r,3r)-3-aminocyclobutane-1-carbonyl)piperazin-1-yl)(4-((3-(1-(2,2-difluoroethyl)-3-(difluoromethyl)-1H-pyrazol-4-yl)imidazo[1,2-α]pyrazin-8-yl)amino)-2-ethylphenyl)methanone;
(2S,4R)—N—((R)-2-(4-((3-(1-(2,2-difluoroethyl)-3-(trifluoromethyl)-1H-pyrazol-4-yl)imidazo[1,2-α]pyrazin-8-yl)amino)-2-fluoro-6-methylbenzamido)propyl)-4-hydroxypyrrolidine-2-carboxamide;
4-((3-(1-(1-cyanoethyl)-3-(trifluoromethyl)-1H-pyrazol-4-yl)imidazo[1,2-α]pyrazin-8-yl)amino)-2-ethyl-N-(2-(2-morpholinoethoxy)ethyl)benzamide;
4-[[3-[1-(cyanomethyl)-3-(trifluoromethyl)pyrazol-4-yl]imidazo[1,2-α]pyrazin-8-yl]amino]-2-methyl-N-[(1R)-1-methyl-2-oxo-2-[[(3R)-pyrrolidin-3-yl]amino]ethyl]benzamide;
(R)-2-amino-1-(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)propan-1-one;
(R)-2-ethyl-N-(3-(2-(2,2,2-trifluoroacetamido)propanamido)propyl)-4-((3-(3-(trifluoromethyl)-1H-pyrazol-4-yl)imidazo[1,2-α]pyrazin-8-yl)amino)benzamide;
N-[2-[(3-aminocyclobutyl)amino]-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
4-[[3-[1-(cyanomethyl)-3-(trifluoromethyl)pyrazol-4-yl]imidazo[1,2-α]pyrazin-8-yl]amino]-2-methyl-N-[2-oxo-2-[[(3R)-pyrrolidin-3-yl]amino]ethyl]benzamide;
N-[2-(3-aminoazetidin-1-yl)-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
4-[[3-[1-(cyanomethyl)-3-(trifluoromethyl)pyrazol-4-yl]imidazo[1,2-α]pyrazin-8-yl]amino]-N-[2-[2-(dimethylamino)ethylamino]-2-oxo-ethyl]-2-methyl-benzamide;
2-ethyl-N-(2-oxo-2-piperazin-1-yl-ethyl)-4-[[3-[3-(trifluoromethyl)-1H-pyrazol-4-yl]imidazo[1,2-α]pyrazin-8-yl]amino]benzamide;
(2S,4R)—N-(3-(4-((3-(1-(cyanomethyl)-3-(trifluoromethyl)-1H-pyrazol-4-yl)imidazo[1,2-α]pyrazin-8-yl)amino)-2-ethylbenzamido)propyl)-4-hydroxypyrrolidine-2-carboxamide;
(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)(4-hydroxypiperidin-4-yl)methanone;
N-[2-(4-amino-1-piperidyl)-2-oxo-ethyl]-2-ethyl-4-[[3-[3-(trifluoromethyl)-1H-pyrazol-4-yl]imidazo[1,2-α]pyrazin-8-yl]amino]benzamide;
4-[[3-[1-(cyanomethyl)-3-(trifluoromethyl)pyrazol-4-yl]imidazo[1,2-α]pyrazin-8-yl]amino]-2-ethyl-N-[2-oxo-2-[[(3R)-pyrrolidin-3-yl]amino]ethyl]benzamide;
4-[[3-[1-(cyanomethyl)-3-(trifluoromethyl)pyrazol-4-yl]imidazo[1,2-α]pyrazin-8-yl]amino]-2-ethyl-N-[2-oxo-2-[[(3S)-pyrrolidin-3-yl]amino]ethyl]benzamide;
(S)-2-ethyl-N-(3-(2-(2,2,2-trifluoroacetamido)propanamido)propyl)-4-((3-(3-(trifluoromethyl)-1H-pyrazol-4-yl)imidazo[1,2-α]pyrazin-8-yl)amino)benzamide;
N-[2-(2-aminoethylamino)-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
N-[(1R)-2-[(3R)-3-aminopyrrolidin-1-yl]-1-methyl-2-oxo-ethyl]-4-[[3-[1-(cyanomethyl)-3-(trifluoromethyl)pyrazol-4-yl]imidazo[1,2-α]pyrazin-8-yl]amino]-2-methyl-benzamide;
(S)—N—((R)-2-(2-ethyl-4-((3-(3-(trifluoromethyl)-1H-pyrazol-4-yl)imidazo[1,2-α]pyrazin-8-yl)amino)benzamido)propyl)pyrrolidine-2-carboxamide;
(S)-(2-chloro-4-((3-(1-(difluoromethyl)-3-(trifluoromethyl)-1H-pyrazol-4-yl)imidazo[1,2-α]pyrazin-8-yl)amino)phenyl)(6-prolyl-2,6-diazaspiro[3.3]heptan-2-yl)methanone;
(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)(4-hydroxypiperidin-4-yl)methanone;
N-[2-(2-aminoethylamino)-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
2-amino-1-(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)ethan-1-one;
(S)-2-amino-1-(4-(4-((3-(3-(difluoromethyl)-1-(methoxymethyl)-1H-pyrazol-4-yl)imidazo[1,2-α]pyrazin-8-yl)amino)-2-ethylbenzoyl)piperazin-1-yl)propan-1-one;
(R)-2-amino-1-(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)-3-methylbutan-1-one;
(2S,4R)—N—((R)-2-(4-((3-(1-(cyanomethyl)-3-(trifluoromethyl)-1H-pyrazol-4-yl)imidazo[1,2-α]pyrazin-8-yl)amino)-2-fluoro-6-methylbenzamido)propyl)-4-hydroxypyrrolidine-2-carboxamide;
(4-(4-((3-(3-(difluoromethyl)-1-(2-fluoroethyl)-1H-pyrazol-4-yl)imidazo[1,2-α]pyrazin-8-yl)amino)-2-ethylbenzoyl)piperazin-1-yl)(piperidin-4-yl)methanone;
2-amino-1-(4-(4-((3-(3-(difluoromethyl)-1-(methoxymethyl)-1H-pyrazol-4-yl)imidazo[1,2-α]pyrazin-8-yl)amino)-2-ethylbenzoyl)piperazin-1-yl)ethan-1-one;
2-ethyl-N-(2-(2-morpholinoethoxy)ethyl)-4-((3-(3-(trifluoromethyl)-1H-pyrazol-4-yl)imidazo[1,2-α]pyrazin-8-yl)amino)benzamide;
(R)-2-amino-1-(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)-3-methylbutan-1-one;
(4-(4-((3-(1-allyl-3-(difluoromethyl)-1H-pyrazol-4-yl)imidazo[1,2-α]pyrazin-8-yl)amino)-2-ethylbenzoyl)piperazin-1-yl)((2S,4R)-4-hydroxypyrrolidin-2-yl)methanone;
(S)-2-amino-1-(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)propan-1-one;
N-((2-aminocyclopropyl)methyl)-2-ethyl-4-((3-(3-(trifluoromethyl)-1H-pyrazol-4-yl)imidazo[1,2-α]pyrazin-8-yl)amino)benzamide;
(R)-2-amino-1-(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)propan-1-one;
(R)-2-amino-1-(4-(4-((3-(3-(difluoromethyl)-1-(methoxymethyl)-1H-pyrazol-4-yl)imidazo[1,2-α]pyrazin-8-yl)amino)-2-ethylbenzoyl)piperazin-1-yl)propan-1-one;
(S)-2-amino-1-(4-(4-((3-(3-(difluoromethyl)-1-(2-fluoroethyl)-1H-pyrazol-4-yl)imidazo[1,2-α]pyrazin-8-yl)amino)-2-ethylbenzoyl)piperazin-1-yl)propan-1-one;
4-[[3-[1-(cyanomethyl)-3-(trifluoromethyl)pyrazol-4-yl]imidazo[1,2-α]pyrazin-8-yl]amino]-2-methyl-N-[2-oxo-2-[[(3S)-pyrrolidin-3-yl]amino]ethyl]benzamide;
N-[2-(2-aminoethoxy)ethyl]-4-[[3-[1-(cyanomethyl)-3-phenyl-pyrazol-4-yl]imidazo[1,2-α]pyrazin-8-yl]amino]-2-ethyl-benzamide;
(2S,4R)—N—((R)-2-(2-ethyl-4-((3-(3-(trifluoromethyl)-1H-pyrazol-4-yl)imidazo[1,2-α]pyrazin-8-yl)amino)benzamido)propyl)-4-hydroxypyrrolidine-2-carboxamide;
2-fluoro-6-methyl-N-(4-(prop-2-yn-1-ylamino)butyl)-4-((3-(3-(trifluoromethyl)-1H-pyrazol-4-yl)imidazo[1,2-α]pyrazin-8-yl)amino)benzamide;
(2R)-2-amino-1-(4-(4-((3-(1-(but-3-yn-2-yl)-3-(difluoromethyl)-1H-pyrazol-4-yl)imidazo[1,2-α]pyrazin-8-yl)amino)-2-ethylbenzoyl)piperazin-1-yl)propan-1-one;
(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)((2S,4R)-4-hydroxypyrrolidin-2-yl)methanone;
(4-(4-((3-(1-(2,2-difluoroethyl)-3-(difluoromethyl)-1H-pyrazol-4-yl)imidazo[1,2-α]pyrazin-8-yl)amino)-2-ethylbenzoyl)piperazin-1-yl)((2S,4R)-4-hydroxypyrrolidin-2-yl)methanone;
(4-(4-((3-(1-(but-3-yn-2-yl)-3-(difluoromethyl)-1H-pyrazol-4-yl)imidazo[1,2-α]pyrazin-8-yl)amino)-2-ethylbenzoyl)piperazin-1-yl)((2R,4S)-4-hydroxypyrrolidin-2-yl)methanone;
2-amino-1-(4-(4-((3-(3-(difluoromethyl)-1-(2-fluoroethyl)-1H-pyrazol-4-yl)imidazo[1,2-α]pyrazin-8-yl)amino)-2-ethylbenzoyl)piperazin-1-yl)ethan-1-one;
(S)—N-(3-(4-((3-(1-(2-amino-2-oxoethyl)-3-(trifluoromethyl)-1H-pyrazol-4-yl)imidazo[1,2-α]pyrazin-8-yl)amino)-2-ethylbenzamido)propyl)pyrrolidine-2-carboxamide;
N-[(1R)-2-[(3-aminocyclobutyl)amino]-1-methyl-2-oxo-ethyl]-4-[[3-[1-(cyanomethyl)-3-(trifluoromethyl)pyrazol-4-yl]imidazo[1,2-α]pyrazin-8-yl]amino]-2-ethyl-benzamide;
(4-(4-((3-(3-(difluoromethyl)-1-(2-fluoroethyl)-1H-pyrazol-4-yl)imidazo[1,2-α]pyrazin-8-yl)amino)-2-ethylbenzoyl)piperazin-1-yl)(4-hydroxypiperidin-4-yl)methanone;
2-(3-(difluoromethyl)-4-(8-((3-ethyl-4-(4-(piperidine-4-carbonyl)piperazine-1-carbonyl)phenyl)amino)imidazo[1,2-α]pyrazin-3-yl)-1H-pyrazol-1-yl)acetonitrile;
2-amino-1-(4-(4-((3-(3-(difluoromethyl)-1-(prop-2-yn-1-yl)-1H-pyrazol-4-yl)imidazo[1,2-α]pyrazin-8-yl)amino)-2-ethylbenzoyl)piperazin-1-yl)ethan-1-one;
(S)—N-(3-(2-aminopropanamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
(4-(4-((3-(3-(difluoromethyl)-1-(2-fluoroethyl)-1H-pyrazol-4-yl)imidazo[1,2-α]pyrazin-8-yl)amino)-2-ethylbenzoyl)piperazin-1-yl)((2S,4R)-4-hydroxypyrrolidin-2-yl)methanone;
(R)-2-amino-1-(4-(4-((3-(3-(difluoromethyl)-1-(2-fluoroethyl)-1H-pyrazol-4-yl)imidazo[1,2-α]pyrazin-8-yl)amino)-2-ethylbenzoyl)piperazin-1-yl)propan-1-one;
2-(4-(8-((4-(4-(D-valyl)piperazine-1-carbonyl)-3-ethylphenyl)amino)imidazo[1,2-α]pyrazin-3-yl)-3-(difluoromethyl)-1H-pyrazol-1-yl)acetonitrile;
(4-(4-((3-(3-(difluoromethyl)-1-(methoxymethyl)-1H-pyrazol-4-yl)imidazo[1,2-α]pyrazin-8-yl)amino)-2-ethylbenzoyl)piperazin-1-yl)(4-hydroxypyrrolidin-2-yl)methanone;
(2R)-2-amino-1-(4-(4-((3-(1-(but-3-yn-2-yl)-3-(difluoromethyl)-1H-pyrazol-4-yl)imidazo[1,2-α]pyrazin-8-yl)amino)-2-ethylbenzoyl)piperazin-1-yl)-3-methylbutan-1-one;
2-(4-(8-((4-(4-((1r,3r)-3-aminocyclobutane-1-carbonyl)piperazine-1-carbonyl)-3-ethylphenyl)amino)imidazo[1,2-α]pyrazin-3-yl)-3-(difluoromethyl)-1H-pyrazol-1-yl)acetonitrile;
(4-((3-(3-(difluoromethyl)-1-(methoxymethyl)-1H-pyrazol-4-yl)imidazo[1,2-α]pyrazin-8-yl)amino)-2-ethylphenyl)(piperazin-1-yl)methanone;
N-(3-(2-aminoacetamido)propyl)-4-((3-(1-(cyanomethyl)-3-(trifluoromethyl)-1H-pyrazol-4-yl)imidazo[1,2-α]pyrazin-8-yl)amino)-2-ethylbenzamide;
4-[[3-[1-(cyanomethyl)-3-(trifluoromethyl)pyrazol-4-yl]imidazo[1,2-α]pyrazin-8-yl]amino]-2-ethyl-N-[(1R)-1-methyl-2-oxo-2-[[(3R)-pyrrolidin-3-yl]amino]ethyl]benzamide;
N-(2-((1s,3s)-3-aminocyclobutane-1-carboxamido)ethyl)-2-ethyl-4-((3-(3-(trifluoromethyl)-1H-pyrazol-4-yl)imidazo[1,2-α]pyrazin-8-yl)amino)benzamide;
N-[3-[(3-aminocyclobutanecarbonyl)amino]propyl]-4-[[3-[1-(3-aminocyclobutanecarbonyl)-3-(trifluoromethyl)pyrazol-4-yl]imidazo[1,2-α]pyrazin-8-yl]amino]-2-ethyl-benzamide;
(2-fluoro-4-((3-(3-(trifluoromethyl)-1H-pyrazol-4-yl)imidazo[1,2-α]pyrazin-8-yl)amino)phenyl)(piperazin-1-yl)methanone
(4-((1S,3R)-3-aminocyclopentane-1-carbonyl)piperazin-1-yl)(4-((3-(1-(2,2-difluoroethyl)-3-(trifluoromethyl)-1H-pyrazol-4-yl)imidazo[1,2-α]pyrazin-8-yl)amino)-2-fluorophenyl)methanone;
2-(4-(8-((4-(4-((1S,3R)-3-aminocyclopentane-1-carbonyl)piperazine-1-carbonyl)-3-fluorophenyl)amino)imidazo[1,2-α]pyrazin-3-yl)-3-(trifluoromethyl)-1H-pyrazol-1-yl)acetonitrile;
(4-((1S,3R)-3-aminocyclopentane-1-carbonyl)piperazin-1-yl)(4-((3-(1-(cyclopropylmethyl)-3-(trifluoromethyl)-1H-pyrazol-4-yl)imidazo[1,2-α]pyrazin-8-yl)amino)-2-fluorophenyl)methanone;
N-(2-(2-aminoethoxy)ethyl)-4-((3-(1-(2,2-difluoroethyl)-3-(trifluoromethyl)-1H-pyrazol-4-yl)imidazo[1,2-α]pyrazin-8-yl)amino)-2-fluorobenzamide;
N-(2-(2-aminoethoxy)ethyl)-4-((3-(1-(cyanomethyl)-3-(trifluoromethyl)-1H-pyrazol-4-yl)imidazo[1,2-α]pyrazin-8-yl)amino)-2-fluorobenzamide;
N-(2-(2-aminoethoxy)ethyl)-4-((3-(1-(cyclopropylmethyl)-3-(trifluoromethyl)-1H-pyrazol-4-yl)imidazo[1,2-α]pyrazin-8-yl)amino)-2-fluorobenzamide;
2-amino-1-(4-(4-((3-(1-(2,2-difluoroethyl)-3-(trifluoromethyl)-1H-pyrazol-4-yl)imidazo[1,2-α]pyrazin-8-yl)amino)-2-fluorobenzoyl)piperazin-1-yl)ethan-1-one;
2-(4-(8-((3-fluoro-4-(4-glycylpiperazine-1-carbonyl)phenyl)amino)imidazo[1,2-α]pyrazin-3-yl)-3-(trifluoromethyl)-1H-pyrazol-1-yl)acetonitrile;
2-amino-1-(4-(4-((3-(1-(cyclopropylmethyl)-3-(trifluoromethyl)-1H-pyrazol-4-yl)imidazo[1,2-α]pyrazin-8-yl)amino)-2-fluorobenzoyl)piperazin-1-yl)ethan-1-one;
N-(2-((2-((2-aminoethyl)amino)-2-oxoethyl)amino)ethyl)-2-chloro-4-((3-(1-(cyanomethyl)-3-(trifluoromethyl)-1H-pyrazol-4-yl)imidazo[1,2-α]pyrazin-8-yl)amino)benzamide;
N-(2-((2-(azetidin-3-ylamino)-2-oxoethyl)amino)ethyl)-2-chloro-4-((3-(1-(cyanomethyl)-3-(trifluoromethyl)-1H-pyrazol-4-yl)imidazo[1,2-α]pyrazin-8-yl)amino)benzamide;
(S)-2-chloro-4-((3-(1-(cyanomethyl)-3-(trifluoromethyl)-1H-pyrazol-4-yl)imidazo[1,2-α]pyrazin-8-yl)amino)-N-(2-((2-oxo-2-(pyrrolidin-3-ylamino)ethyl)amino)ethyl)benzamide;
(R)-2-chloro-4-((3-(1-(cyanomethyl)-3-(trifluoromethyl)-1H-pyrazol-4-yl)imidazo[1,2-α]pyrazin-8-yl)amino)-N-(2-((2-oxo-2-(pyrrolidin-3-ylamino)ethyl)amino)ethyl)benzamide;
N-(2-((2-((3-aminopropyl)amino)-2-oxoethyl)amino)ethyl)-2-chloro-4-((3-(1-(cyanomethyl)-3-(trifluoromethyl)-1H-pyrazol-4-yl)imidazo[1,2-α]pyrazin-8-yl)amino)benzamide;
N-(2-((2-((2-aminoethyl)amino)-2-oxoethyl)amino)ethyl)-2-chloro-4-((3-(1-(2,2-difluoroethyl)-3-(trifluoromethyl)-1H-pyrazol-4-yl)imidazo[1,2-α]pyrazin-8-yl)amino)benzamide;
N-(2-((2-(azetidin-3-ylamino)-2-oxoethyl)amino)ethyl)-2-chloro-4-((3-(1-(2,2-difluoroethyl)-3-(trifluoromethyl)-1H-pyrazol-4-yl)imidazo[1,2-α]pyrazin-8-yl)amino)benzamide;
(S)-2-chloro-4-((3-(1-(2,2-difluoroethyl)-3-(trifluoromethyl)-1H-pyrazol-4-yl)imidazo[1,2-α]pyrazin-8-yl)amino)-N-(2-((2-oxo-2-(pyrrolidin-3-ylamino)ethyl)amino)ethyl)benzamide;
(R)-2-chloro-4-((3-(1-(2,2-difluoroethyl)-3-(trifluoromethyl)-1H-pyrazol-4-yl)imidazo[1,2-α]pyrazin-8-yl)amino)-N-(2-((2-oxo-2-(pyrrolidin-3-ylamino)ethyl)amino)ethyl)benzamide;
N-(2-((2-((3-aminopropyl)amino)-2-oxoethyl)amino)ethyl)-2-chloro-4-((3-(1-(2,2-difluoroethyl)-3-(trifluoromethyl)-1H-pyrazol-4-yl)imidazo[1,2-α]pyrazin-8-yl)amino)benzamide;
(1S,5R)-6-[[2-chloro-4-[[3-[1-(cyanomethyl)-3-(trifluoromethyl)pyrazol-4-yl]imidazo[1,2-α]pyrazin-8-yl]amino]benzoyl]amino]-N!-[(3R,4R)-4-hydroxypyrrolidin-3-yl]-3-azabicyclo[3.1.0]hexane-3-carboxamide;
N-(6-aminohexyl)-2-chloro-4-((3-(1-(2,2-difluoroethyl)-3-(trifluoromethyl)-1H-pyrazol-4-yl)imidazo[1,2-α]pyrazin-8-yl)amino)benzamide;
N-((1-(azetidin-3-ylmethyl)pyrrolidin-3-yl)methyl)-2-ethyl-4-((3-(3-(trifluoromethyl)-1H-pyrazol-4-yl)imidazo[1,2-α]pyrazin-8-yl)amino)benzamide;
N-((1-(azetidin-3-ylmethyl)piperidin-4-yl)methyl)-4-((3-(1-(cyanomethyl)-3-(trifluoromethyl)-1H-pyrazol-4-yl)imidazo[1,2-α]pyrazin-8-yl)amino)-2-ethylbenzamide;
2-[azetidin-3-ylmethyl-[5-[[4-[[3-[1-(2,2-difluoroethyl)-3-(trifluoromethyl)pyrazol-4-yl]imidazo[1,2-α]pyrazin-8-yl]amino]-2-ethyl-benzoyl]amino]pentyl]amino]acetic acid;
N-[2-(2-aminoethoxy)ethyl]-4-[[3-[3-cyano-1-(cyanomethyl)pyrazol-4-yl]imidazo[1,2-α]pyrazin-8-yl]amino]-2-ethyl-benzamide;
N-[2-(2-aminoethoxy)ethyl]-4-[[3-[3-cyano-1-(2,2-difluoroethyl)pyrazol-4-yl]imidazo[1,2-α]pyrazin-8-yl]amino]-2-ethyl-benzamide;
N-[2-(2-aminoethoxy)ethyl]-4-[[3-[1-(cyanomethyl)-3-methylsulfanyl-pyrazol-4-yl]imidazo[1,2-α]pyrazin-8-yl]amino]-2-ethyl-benzamide; and
N-[2-(2-aminoethoxy)ethyl]-4-[[3-[3-chloro-1-(cyanomethyl)pyrazol-4-yl]imidazo[1,2-α]pyrazin-8-yl]amino]-2-ethyl-benzamide.

58. A process of manufacturing the compounds of formula (I) according to any one of claims 1 to 57, comprising:
(i) reacting a carboxylic acid IXa, wherein R³ to R⁹ are as defined herein, 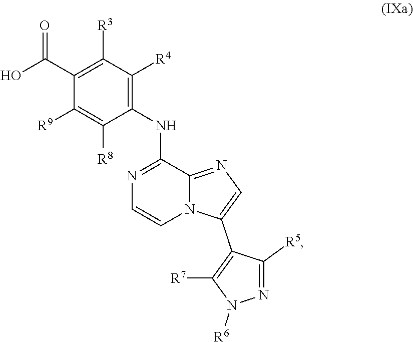
with an amine V, wherein R¹ and R² are as defined herein, 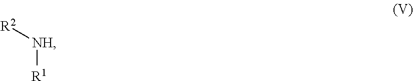
to form said compound of formula (I); or
(ii) reacting a compound VIa, wherein R¹ to R⁴, R⁸, and R⁹ are as defined herein and Z is a halogen or a triflate, 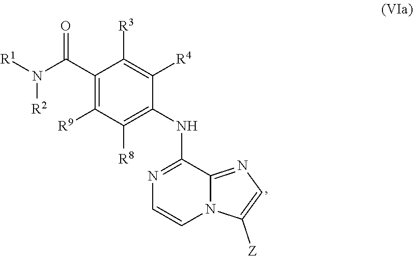
with a compound VIIa, wherein R⁵ to R⁷ are as defined herein and Y is a boronic acid or a boronic acid ester, 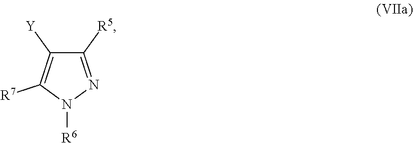
to form said compound of formula (I).

59. A compound of formula (I) according to any one of claims 1 to 57, when manufactured according to the process of claim 58.

60. A compound of formula (I) according to any one of claims 1 to 57 and 59, or a pharmaceutically acceptable salt thereof, for use as therapeutically active substance.

61. A pharmaceutical composition comprising a compound of formula (I) according to any one of claims 1 to 57 and 59, or a pharmaceutically acceptable salt thereof, and a therapeutically inert carrier.

62. A compound of formula (I) according to any of claims 1 to 57 and 59, or a pharmaceutically acceptable salt thereof, for use as antibiotic.

63. A compound of formula (I) according to any of claims 1 to 57 and 59, or a pharmaceutically acceptable salt thereof, for use in the treatment or prevention of nosocomial infections and resulting diseases.

64. A compound of formula (I) according to any of claims 1 to 57 and 59, or a pharmaceutically acceptable salt thereof, for use in the treatment or prevention of infections and resulting diseases caused by Gram-negative bacteria.

65. The compound for use according to claim 64, wherein said Gram-negative bacteria are selected from Klebsiella pneumoniae, Acinetobacter baumannii, Pseudomonas aeruginosa, Enterobacter species and E. coli.

66. The compound for use according to claim 65, wherein said Gram-negative bacteria are Acinetobacter baumannii.

67. A compound of formula (I) according to any of claims 1 to 57 and 59, or a pharmaceutically acceptable salt thereof, for use in the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

68. A method for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof, which method comprises administering a compound of formula (I) according to any of claims 1 to 57 and 59, or a pharmaceutically acceptable salt thereof, to a mammal.

69. Use of a compound of formula (I) according to any of claims 1 to 57 and 59, or a pharmaceutically acceptable salt thereof, as an antibiotic.

70. Use of a compound of formula (I) according to any of claims 1 to 57 and 59, or a pharmaceutically acceptable salt thereof, for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

71. The use of a compound of formula (I) according to any of claims 1 to 57 and 59, or a pharmaceutically acceptable salt thereof, for the preparation of medicaments useful for the treatment or prevention of infections and resulting diseases caused by Enterococcus faecium, Staphylococcus aureus, Klebsiella pneumoniae, Acinetobacter baumannii, Pseudomonas aeruginosa, Enterobacter species or E. coli, or a combination thereof.

72. The invention as described hereinbefore.
